SEQUENCE LISTING (417 sequences)

SEQ ID NO: 1 - protein, 21 aa, synthetic construct
  REGION 1..21: note = Description of Artificial Sequence: Synthetic peptide
MALPVTALLLPLALLLHAARP

SEQ ID NO: 2 - protein, 45 aa, synthetic construct
  REGION 1..45: note = Description of Artificial Sequence: Synthetic polypeptide
TTTPAPRPPTPAPTIASQPLSLRPEACRPAAGGAVHTRGLDFACD

SEQ ID NO: 3 - protein, 230 aa, synthetic construct
  REGION 1..230: note = Description of Artificial Sequence: Synthetic polypeptide
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGKM

SEQ ID NO: 4 - protein, 282 aa, synthetic construct
  REGION 1..282: note = Description of Artificial Sequence: Synthetic polypeptide
RWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDH

SEQ ID NO: 5 - protein, 10 aa, synthetic construct
  REGION 1..10: note = Description of Artificial Sequence: Synthetic peptide
GGGGSGGGGS

SEQ ID NO: 6 - protein, 24 aa, synthetic construct
  REGION 1..24: note = Description of Artificial Sequence: Synthetic peptide
IYIWAPLAGTCGVLLLSLVITLYC

SEQ ID NO: 7 - protein, 42 aa, synthetic construct
  REGION 1..42: note = Description of Artificial Sequence: Synthetic polypeptide
KRGRKKLLYIFKQPFMRPVQTTQEEDGCSCRFPEEEEGGCEL

SEQ ID NO: 8 - protein, 48 aa, synthetic construct
  REGION 1..48: note = Description of Artificial Sequence: Synthetic polypeptide
QRRKYRSNKGESPVEPAEPCRYSCPREEEGSTIPIQEDYRKPEPACSP

SEQ ID NO: 9 - protein, 112 aa, synthetic construct
  REGION 1..112: note = Description of Artificial Sequence: Synthetic polypeptide
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 1184 bp, synthetic construct
  misc_feature 1..1184: note = Description of Artificial Sequence: Synthetic polynucleotide
  source 1..1184: mol_type = other DNA
cgtgaggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtga

SEQ ID NO: 12 - DNA, 63 bp, synthetic construct
  misc_feature 1..63: note = Description of Artificial Sequence: Synthetic oligonucleotide
  source 1..63: mol_type = other DNA
atggccctgcctgtgacagccctgctgctgcctctggctctgctgctgcatgccgctagaccc

SEQ ID NO: 13 - DNA, 135 bp, synthetic construct
  misc_feature 1..135: note = Description of Artificial Sequence: Synthetic polynucleotide
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 14 - DNA, 690 bp, synthetic construct
  misc_feature 1..690: note = Description of Artificial Sequence: Synthetic polynucleotide
  source 1..690: mol_type = other DNA
gagagcaagtacggccctccctgccccccttgccctgcccccgagttcctgggcggacccagcgtgttcctgttcccccccaagcccaaggacaccctgatgatcagccggacccccgaggtgacctgtgtggtggtggacgtgtcccaggaggaccccgaggtccagttcaactggtacgtggacggcgtggaggtgcacaacgccaagaccaagccccgggaggagcagttcaatagcacctaccgggtggtgtccgtgctgaccgtgctgcaccaggactggctgaacggcaaggaatacaagtgtaaggtgtccaacaagggcctgcccagcagcatcgagaaaaccatcagcaaggccaagggccagcctcgggagccccaggtgtacaccctgccccctagccaagaggagatgaccaagaaccaggtgtccctgacctgcctggtgaagggcttctaccccagcgacatcgccgtggagtgggagagcaacggccagcccgagaacaactacaagaccaccccccctgtgctggacagcgacggcagcttcttcctgtacagccggctgaccgtggacaagagccggtggcaggagggcaacgtctttagctgctccgtgatgcacgaggccctgcacaaccactacacccagaagagcctgagcctgtccctgggcaagatg

SEQ ID NO: 15 - DNA, 847 bp, synthetic construct
  misc_feature 1..847: note = Description of Artificial Sequence: Synthetic polynucleotide
  source 1..847: mol_type = other DNA
aggtggcccgaaagtcccaaggcccaggcatctagtgttcctactgcacagccccaggcagaaggcagcctagccaaagctactactgcacctgccactacgcgcaatactggccgtggcggggaggagaagaaaaaggagaaagagaaagaagaacaggaagagagggagaccaagacccctgaatgtccatcccatacccagccgctgggcgtctatctcttgactcccgcagtacaggacttgtggcttagagataaggccacctttacatgtttcgtcgtgggctctgacctgaaggatgcccatttgacttgggaggttgccggaaaggtacccacagggggggttgaggaagggttgctggagcgccattccaatggctctcagagccagcactcaagactcacccttccgagatccctgtggaacgccgggacctctgtcacatgtactctaaatcatcctagcctgcccccacagcgtctgatggcccttagagagccagccgcccaggcaccagttaagcttagcctgaatctgctcgccagtagtgatcccccagaggccgccagctggctcttatgcgaagtgtccggctttagcccgcccaacatcttgctcatgtggctggaggaccagcgagaagtgaacaccagcggcttcgctccagcccggcccccaccccagccgggttctaccacattctgggcctggagtgtcttaagggtcccagcaccacctagcccccagccagccacatacacctgtgttgtgtcccatgaagatagcaggaccctgctaaatgcttctaggagtctggaggtttcctacgtgactgaccatt

SEQ ID NO: 16 - DNA, 30 bp, synthetic construct
  misc_feature 1..30: note = Description of Artificial Sequence: Synthetic oligonucleotide
  source 1..30: mol_type = other DNA
ggtggcggaggttctggaggtggaggttcc

SEQ ID NO: 17 - DNA, 72 bp, synthetic construct
  misc_feature 1..72: note = Description of Artificial Sequence: Synthetic oligonucleotide
  source 1..72: mol_type = other DNA
atctacatctgggcgcccttggccgggacttgtggggtccttctcctgtcactggttatcaccctttactgc

SEQ ID NO: 18 - DNA, 126 bp, synthetic construct
  misc_feature 1..126: note = Description of Artificial Sequence: Synthetic polynucleotide
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 19 - DNA, 123 bp, synthetic construct
  misc_feature 1..123: note = Description of Artificial Sequence: Synthetic polynucleotide
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 20 - DNA, 336 bp, synthetic construct
  misc_feature 1..336: note = Description of Artificial Sequence: Synthetic polynucleotide
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 21 - DNA, 336 bp, Homo sapiens
  source 1..336: mol_type = genomic DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 22 - protein, 373 aa, synthetic construct
  REGION 1..373: note = Description of Artificial Sequence: Synthetic polypeptide
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 23 - DNA, 1182 bp, synthetic construct
  misc_feature 1..1182: note = Description of Artificial Sequence: Synthetic polynucleotide
  source 1..1182: mol_type = other DNA
atggccctccctgtcactgccctgcttctccccctcgcactcctgctccacgccgctagaccacccggatggtttctggactctccggatcgcccgtggaatcccccaaccttctcaccggcactcttggttgtgactgagggcgataatgcgaccttcacgtgctcgttctccaacacctccgaatcattcgtgctgaactggtaccgcatgagcccgtcaaaccagaccgacaagctcgccgcgtttccggaagatcggtcgcaaccgggacaggattgtcggttccgcgtgactcaactgccgaatggcagagacttccacatgagcgtggtccgcgctaggcgaaacgactccgggacctacctgtgcggagccatctcgctggcgcctaaggcccaaatcaaagagagcttgagggccgaactgagagtgaccgagcgcagagctgaggtgccaactgcacatccatccccatcgcctcggcctgcggggcagtttcagaccctggtcacgaccactccggcgccgcgcccaccgactccggccccaactatcgcgagccagcccctgtcgctgaggccggaagcatgccgccctgccgccggaggtgctgtgcatacccggggattggacttcgcatgcgacatctacatttgggctcctctcgccggaacttgtggcgtgctccttctgtccctggtcatcaccctgtactgcaagcggggtcggaaaaagcttctgtacattttcaagcagcccttcatgaggcccgtgcaaaccacccaggaggaggacggttgctcctgccggttccccgaagaggaagaaggaggttgcgagctgcgcgtgaagttctcccggagcgccgacgcccccgcctataagcagggccagaaccagctgtacaacgaactgaacctgggacggcgggaagagtacgatgtgctggacaagcggcgcggccgggaccccgaaatgggcgggaagcctagaagaaagaaccctcaggaaggcctgtataacgagctgcagaaggacaagatggccgaggcctactccgaaattgggatgaagggagagcggcggaggggaaaggggcacgacggcctgtaccaaggactgtccaccgccaccaaggacacatacgatgccctgcacatgcaggcccttccccctcgc

SEQ ID NO: 24 - protein, 394 aa, synthetic construct
  REGION 1..394: note = Description of Artificial Sequence: Synthetic polypeptide
MALPVTALLLPLALLLHAARPPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 25 - protein, 5 aa, synthetic construct
  REGION 1..5: note = Description of Artificial Sequence: Synthetic peptide
GGGGS

SEQ ID NO: 26 - protein, 30 aa, synthetic construct
  REGION 1..30: note = Description of Artificial Sequence: Synthetic polypeptide
  REGION 1..30: note = MISC_FEATURE - This sequence may encompass 1-6 "Gly Gly Gly Gly Ser" repeating units
  REGION 1..30: note = See specification as filed for detailed description of substitutions and preferred embodiments
GGGGSGGGGSGGGGSGGGGSGGGGSGGGGS

SEQ ID NO: 27 - protein, 20 aa, synthetic construct
  REGION 1..20: note = Description of Artificial Sequence: Synthetic peptide
GGGGSGGGGSGGGGSGGGGS

SEQ ID NO: 28 - protein, 15 aa, synthetic construct
  REGION 1..15: note = Description of Artificial Sequence: Synthetic peptide
GGGGSGGGGSGGGGS

SEQ ID NO: 29 - protein, 4 aa, synthetic construct
  REGION 1..4: note = Description of Artificial Sequence: Synthetic peptide
GGGS

SEQ ID NO: 30 - RNA, 5000 nt, synthetic construct
  misc_feature 1..5000: note = Description of Artificial Sequence: Synthetic polynucleotide
  misc_feature 1..5000: note = This sequence may encompass 50-5000 nucleotides
  source 1..5000: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 31 - DNA, 100 bp, synthetic construct
  misc_feature 1..100: note = Description of Artificial Sequence: Synthetic polynucleotide
  source 1..100: mol_type = other DNA
tttttttttttttttttttttttttttttttttttttttttttttttttttttttttttttttttttttttttttttttttttttttttttttttttttt

SEQ ID NO: 32 - DNA, 5000 bp, synthetic construct
  misc_feature 1..5000: note = Description of Artificial Sequence: Synthetic polynucleotide
  misc_feature 1..5000: note = This sequence may encompass 50-5000 nucleotides
  source 1..5000: mol_type = other DNA
tttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttttt

SEQ ID NO: 33 - RNA, 5000 nt, synthetic construct; identical to SEQ ID NO: 30
  misc_feature 1..5000: note = Description of Artificial Sequence: Synthetic polynucleotide
  misc_feature 1..5000: note = This sequence may encompass 100-5000 nucleotides
  source 1..5000: mol_type = other RNA

SEQ ID NO: 34 - RNA, 400 nt, synthetic construct
  misc_feature 1..400: note = Description of Artificial Sequence: Synthetic polynucleotide
  misc_feature 1..400: note = This sequence may encompass 100-400 nucleotides
  source 1..400: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 35 - RNA, 2000 nt, synthetic construct
  misc_feature 1..2000: note = Description of Artificial Sequence: Synthetic polynucleotide
  misc_feature 1..2000: note = This sequence may encompass 50-2000 nucleotides
  source 1..2000: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 36 - protein, 41 aa, synthetic construct
  REGION 1..41: note = Description of Artificial Sequence: Synthetic polypeptide
RSKRSRLLHSDYMNMTPRRPGPTRKHYQPYAPPRDFAAYRS

SEQ ID NO: 37 - DNA, 123 bp, synthetic construct; identical to SEQ ID NO: 19
  misc_feature 1..123: note = Description of Artificial Sequence: Synthetic polynucleotide
  source 1..123: mol_type = other DNA

SEQ ID NO: 38 - protein, 35 aa, synthetic construct
  REGION 1..35: note = Description of Artificial Sequence: Synthetic polypeptide
TKKKYSSSVHDPNGEYMFMRAVNTAKKSRLTDVTL

SEQ ID NO: 39 - DNA, 105 bp, synthetic construct
  misc_feature 1..105: note = Description of Artificial Sequence: Synthetic polynucleotide
  source 1..105: mol_type = other DNA
acaaaaaagaagtattcatccagtgtgcacgaccctaacggtgaatacatgttcatgagagcagtgaacacagccaaaaaatccagactcacagatgtgacccta

SEQ ID NO: 40 - DNA, 30 bp, synthetic construct
  misc_feature 1..30: note = Description of Artificial Sequence: Synthetic oligonucleotide
  source 1..30: mol_type = other DNA
ggtggcggaggttctggaggtgggggttcc

SEQ ID NO: 41 - protein, 4 aa, synthetic construct
  REGION 1..4: note = Description of Artificial Sequence: Synthetic peptide
GGGS

SEQ ID NO: 42 - protein, 40 aa, synthetic construct
  REGION 1..40: note = Description of Artificial Sequence: Synthetic polypeptide
  REGION 1..40: note = MISC_FEATURE - This sequence may encompass 1-10 "Gly Gly Gly Ser" repeating units
GGGSGGGSGGGSGGGSGGGSGGGSGGGSGGGSGGGSGGGS

SEQ ID NO: 43 - protein, 18 aa, synthetic construct
  REGION 1..18: note = Description of Artificial Sequence: Synthetic peptide
GSTSGSGKPGSGEGSTKG

SEQ ID NO: 44 - protein, 5 aa, synthetic construct
  REGION 1..5: note = Description of Artificial Sequence: Synthetic peptide
SYAMS

SEQ ID NO: 45 - protein, 17 aa, synthetic construct
  REGION 1..17: note = Description of Artificial Sequence: Synthetic peptide
AISGSGGSTYYADSVKG

SEQ ID NO: 46 - protein, 14 aa, synthetic construct
  REGION 1..14: note = Description of Artificial Sequence: Synthetic peptide
REWVPYDVSWYFDY

SEQ ID NO: 47 - protein, 7 aa, synthetic construct
  REGION 1..7: note = Description of Artificial Sequence: Synthetic peptide
GFTFSSY

SEQ ID NO: 48 - protein, 6 aa, synthetic construct
  REGION 1..6: note = Description of Artificial Sequence: Synthetic peptide
SGSGGS

SEQ ID NO: 49 - protein, 8 aa, synthetic construct
  REGION 1..8: note = Description of Artificial Sequence: Synthetic peptide
GFTFSSYA

SEQ ID NO: 50 - protein, 8 aa, synthetic construct
  REGION 1..8: note = Description of Artificial Sequence: Synthetic peptide
ISGSGGST

SEQ ID NO: 51 - protein, 16 aa, synthetic construct
  REGION 1..16: note = Description of Artificial Sequence: Synthetic peptide
ARREWVPYDVSWYFDY

SEQ ID NO: 52 - protein, 123 aa, synthetic construct
  REGION 1..123: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSS

SEQ ID NO: 53 - DNA, 369 bp, synthetic construct
  misc_feature 1..369: note = Description of Artificial Sequence: Synthetic polynucleotide
  source 1..369: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctcc

SEQ ID NO: 54 - protein, 11 aa, synthetic construct
  REGION 1..11: note = Description of Artificial Sequence: Synthetic peptide
RASQSISSYLN

SEQ ID NO: 55 - protein, 7 aa, synthetic construct
  REGION 1..7: note = Description of Artificial Sequence: Synthetic peptide
AASSLQS

SEQ ID NO: 56 - protein, 9 aa, synthetic construct
  REGION 1..9: note = Description of Artificial Sequence: Synthetic peptide
QQSYSTPLT

SEQ ID NO: 57 - protein, 7 aa, synthetic construct
  REGION 1..7: note = Description of Artificial Sequence: Synthetic peptide
SQSISSY

SEQ ID NO: 58 - skipped

SEQ ID NO: 59 - protein, 6 aa, synthetic construct
  REGION 1..6: note = Description of Artificial Sequence: Synthetic peptide
SYSTPL

SEQ ID NO: 60 - protein, 6 aa, synthetic construct
  REGION 1..6: note = Description of Artificial Sequence: Synthetic peptide
QSISSY

SEQ ID NO: 61 - protein, 107 aa, synthetic construct [sequence omitted]

SEQ ID NO: 62 - DNA, 321 bp, synthetic construct
  misc_feature 1..321: note = Description of Artificial Sequence: Synthetic polynucleotide
  source 1..321: mol_type = other DNA
gacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 63 - protein, 20 aa, synthetic construct
  REGION 1..20: note = Description of Artificial Sequence: Synthetic peptide
GGGGSGGGGSGGGGSGGGGS

SEQ ID NO: 64 - protein, 250 aa, synthetic construct
  REGION 1..250: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 65 - DNA, 750 bp, synthetic construct
  misc_feature 1..750: note = Description of Artificial Sequence: Synthetic polynucleotide
  source 1..750: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 66 - protein, 473 aa, synthetic construct
  REGION 1..473: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 67 - DNA, 1419 bp, synthetic construct
  misc_feature 1..1419: note = Description of Artificial Sequence: Synthetic polynucleotide
  source 1..1419: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 68 - protein, 12 aa, synthetic construct
  REGION 1..12: note = Description of Artificial Sequence: Synthetic peptide
REWWYDDWYLDY

SEQ ID NO: 69 - protein, 14 aa, synthetic construct
  REGION 1..14: note = Description of Artificial Sequence: Synthetic peptide
ARREWWYDDWYLDY

SEQ ID NO: 70 - protein, 121 aa, synthetic construct
  REGION 1..121: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSS

SEQ ID NO: 71 - DNA, 363 bp, synthetic construct
  misc_feature 1..363: note = Description of Artificial Sequence: Synthetic polynucleotide
  source 1..363: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctcc

SEQ ID NO: 72 - protein, 248 aa, synthetic construct
  REGION 1..248: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 73 - DNA, 744 bp, synthetic construct
  misc_feature 1..744: note = Description of Artificial Sequence: Synthetic polynucleotide
  source 1..744: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 74 - protein, 471 aa, synthetic construct
  REGION 1..471: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWYDDWYLDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 75 - DNA, 1413 bp, synthetic construct
  misc_feature 1..1413: note = Description of Artificial Sequence: Synthetic polynucleotide
  source 1..1413: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggtacgacgattggtacctggactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 76 - protein, 12 aa, synthetic construct
  REGION 1..12: note = Description of Artificial Sequence: Synthetic peptide
REWWGESWLFDY

SEQ ID NO: 77 - protein, 14 aa, synthetic construct
  REGION 1..14: note = Description of Artificial Sequence: Synthetic peptide
ARREWWGESWLFDY

SEQ ID NO: 78 - protein, 121 aa, synthetic construct
  REGION 1..121: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSS

SEQ ID NO: 79 - DNA, 363 bp, synthetic construct
  misc_feature 1..363: note = Description of Artificial Sequence: Synthetic polynucleotide
  source 1..363: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctcc

SEQ ID NO: 80 - protein, 248 aa, synthetic construct
  REGION 1..248: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIK

SEQ ID NO: 81 - DNA, 744 bp, synthetic construct
  misc_feature 1..744: note = Description of Artificial Sequence: Synthetic polynucleotide
  source 1..744: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaag

SEQ ID NO: 82 - protein, 471 aa, synthetic construct
  REGION 1..471: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 83 - DNA, 1413 bp, synthetic construct
  misc_feature 1..1413: note = Description of Artificial Sequence: Synthetic polynucleotide
  source 1..1413: mol_type = other DNA
gaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 84 - protein, 14 aa, synthetic construct
  REGION 1..14: note = Description of Artificial Sequence: Synthetic peptide
  MOD_RES 4: note = V or absent
  MOD_RES 5: note = P or absent
  MOD_RES 6: note = W or Y
  MOD_RES 7: note = G, Y, or D
  MOD_RES 8: note = E, D, or V
  MOD_RES 9: note = S or D
  MOD_RES 11: note = L or Y
  MOD_RES 12: note = F or L
REWXXXXXXWXXDY

SEQ ID NO: 85 - protein, 16 aa, synthetic construct
  REGION 1..16: note = Description of Artificial Sequence: Synthetic peptide
  MOD_RES 6: note = V or absent
  MOD_RES 7: note = P or absent
  MOD_RES 8: note = W or Y
  MOD_RES 9: note = G, Y, or D
  MOD_RES 10: note = E, D, or V
  MOD_RES 11: note = S or D
  MOD_RES 13: note = L or Y
  MOD_RES 14: note = F or L
ARREWXXXXXXWXXDY

SEQ ID NO: 86 - protein, 5 aa, synthetic construct
  REGION 1..5: note = Description of Artificial Sequence: Synthetic peptide
SYGMH

SEQ ID NO: 87 - protein, 17 aa, synthetic construct
  REGION 1..17: note = Description of Artificial Sequence: Synthetic peptide
VISYDGSNKYYADSVKG

SEQ ID NO: 88 - protein, 14 aa, synthetic construct
  REGION 1..14: note = Description of Artificial Sequence: Synthetic peptide
SGYALHDDYYGLDV

SEQ ID NO: 89 - protein, 6 aa, synthetic construct
  REGION 1..6: note = Description of Artificial Sequence: Synthetic peptide
SYDGSN

SEQ ID NO: 90 - protein, 8 aa, synthetic construct
  REGION 1..8: note = Description of Artificial Sequence: Synthetic peptide
GFTFSSYG

SEQ ID NO: 91 - protein, 8 aa, synthetic construct
  REGION 1..8: note = Description of Artificial Sequence: Synthetic peptide
ISYDGSNK

SEQ ID NO: 92 - protein, 16 aa, synthetic construct
  REGION 1..16: note = Description of Artificial Sequence: Synthetic peptide
GGSGYALHDDYYGLDV

SEQ ID NO: 93 - protein, 123 aa, synthetic construct
  REGION 1..123: note = Description of Artificial Sequence: Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSS

SEQ ID NO: 94 - DNA, 369 bp, synthetic construct
  misc_feature 1..369: note = Description of Artificial Sequence: Synthetic polynucleotide
  source 1..369: mol_type = other DNA
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagc

SEQ ID NO: 95 - protein, 14 aa, synthetic construct
  REGION 1..14: note = Description of Artificial Sequence: Synthetic peptide
TGTSSDVGGYNYVS

SEQ ID NO: 96 - protein, 7 aa, synthetic construct
  REGION 1..7: note = Description of Artificial Sequence: Synthetic peptide
DVSNRPS

SEQ ID NO: 97 - protein, 11 aa, synthetic construct
  REGION 1..11: note = Description of Artificial Sequence: Synthetic peptide
SSYTSSSTLYV

SEQ ID NO: 98 - protein, 10 aa, synthetic construct
  REGION 1..10: note = Description of Artificial Sequence: Synthetic peptide
TSSDVGGYNY

SEQ ID NO: 99 - skipped

SEQ ID NO: 100 - protein, 8 aa, synthetic construct
  REGION 1..8: note = Description of Artificial Sequence: Synthetic peptide
YTSSSTLY

SEQ ID NO: 101 - protein, 9 aa, synthetic construct
  REGION 1..9: note = Description of Artificial Sequence: Synthetic peptide
SSDVGGYNY

SEQ ID NO: 102 - protein, 111 aa, synthetic construct
  REGION 1..111: note = Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 103 - DNA, 333 bp, synthetic construct
  misc_feature 1..333: note = Description of Artificial Sequence: Synthetic polynucleotide
  source 1..333: mol_type = other DNA
cagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 104 - protein, 15 aa, synthetic construct
  REGION 1..15: note = Description of Artificial Sequence: Synthetic peptide
GGGGSGGGGSGGGGS

SEQ ID NO: 105 - protein, 249 aa, synthetic construct
  REGION 1..249: note = Description of Artificial Sequence: Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVL

SEQ ID NO: 106 - DNA, 747 bp, synthetic construct
  misc_feature 1..747: note = Description of Artificial Sequence: Synthetic polynucleotide
  source 1..747: mol_type = other DNA
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctg

SEQ ID NO: 107 - protein, 472 aa, synthetic construct
  REGION 1..472: note = Description of Artificial Sequence: Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 108 - DNA, 1416 bp, synthetic construct
  misc_feature 1..1416: note = Description of Artificial Sequence: Synthetic polynucleotide
  source 1..1416: mol_type = other DNA
caagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 109 - protein, 17 aa, synthetic construct
  REGION 1..17: note = Description of Artificial Sequence: Synthetic peptide
VISYKGSNKYYADSVKG

SEQ ID NO: 110 - protein, 6 aa, synthetic construct
  REGION 1..6: note = Description of Artificial Sequence: Synthetic peptide
SYKGSN

SEQ ID NO: 111 - protein, 8 aa, synthetic construct
  REGION 1..8: note = Description of Artificial Sequence: Synthetic peptide
ISYKGSNK

SEQ ID NO: 112 - protein, 123 aa, synthetic construct
  REGION 1..123: note = Description of Artificial Sequence: Synthetic polypeptide
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSS

SEQ ID NO: 113 - DNA, 369 bp, synthetic construct
  misc_feature 1..369: note = Description of Artificial Sequence: Synthetic polynucleotide
  source 1..369: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctct

SEQ ID NO: 114 - protein, 7 aa, synthetic construct
  REGION 1..7: note = Description of Artificial Sequence: Synthetic peptide
EVSNRLR

SEQ ID NO: 115 - protein, 11 aa, synthetic construct
  REGION 1..11: note = Description of Artificial Sequence: Synthetic peptide
SSYTSSSALYV

SEQ ID NO: 116 - skipped

SEQ ID NO: 117 - protein, 8 aa, synthetic construct
  REGION 1..8: note = Description of Artificial Sequence: Synthetic peptide
YTSSSALY

SEQ ID NO: 118 - protein, 111 aa, synthetic construct
  REGION 1..111: note = Description of Artificial Sequence: Synthetic polypeptide
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 119 - DNA, 333 bp, synthetic construct
  misc_feature 1..333: note = Description of Artificial Sequence: Synthetic polynucleotide
  source 1..333: mol_type = other DNA
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 120 - protein, 254 aa, synthetic construct
  REGION 1..254: note = Description of Artificial Sequence: Synthetic polypeptide
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVL

SEQ ID NO: 121 - DNA, 762 bp, synthetic construct
  misc_feature 1..762: note = Description of Artificial Sequence: Synthetic polynucleotide
  source 1..762: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctg

SEQ ID NO: 122 - protein, 477 aa, synthetic construct
  REGION 1..477: note = Description of Artificial Sequence: Synthetic polypeptide
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSALYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 123 - DNA, 1431 bp, synthetic construct
  misc_feature 1..1431: note = Description of Artificial Sequence: Synthetic polynucleotide
  source 1..1431: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccgccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 124 - protein, 111 aa, synthetic construct
  REGION 1..111: note = Description of Artificial Sequence: Synthetic polypeptide
QSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 125 - DNA, 333 bp, synthetic construct
  misc_feature 1..333: note = Description of Artificial Sequence: Synthetic polynucleotide
  source 1..333: mol_type = other DNA
cagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 126 - protein, 254 aa, synthetic construct
  REGION 1..254: note = Description of Artificial Sequence: Synthetic polypeptide
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVL

SEQ ID NO: 127 - DNA, 762 bp, synthetic construct
  misc_feature 1..762: note = Description of Artificial Sequence: Synthetic polynucleotide
  source 1..762: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctg

SEQ ID NO: 128 - protein, 477 aa, synthetic construct
  REGION 1..477: note = Description of Artificial Sequence: Synthetic polypeptide
QVQLVESGGGVVQPGRSLRLSCAASGFTFSSYGMHWVRQAPGKGLEWVAVISYKGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYEVSNRLRGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 129 - DNA, 1431 bp, synthetic construct
  misc_feature 1..1431: note = Description of Artificial Sequence: Synthetic polynucleotide
  source 1..1431: mol_type = other DNA
caagtgcagcttgtcgaatcgggaggcggagtggtgcagcctggacgatcgctccggctctcatgtgccgcgagcggattcaccttctcgagctacggcatgcactgggtcagacaagccccaggaaagggcctggaatgggtggctgtcatctcgtacaagggctcaaacaagtactacgccgactccgtgaagggccggttcaccatctcccgcgataactccaagaataccctctatctgcaaatgaacagcctgagggccgaggatactgcagtgtactactgcgggggttcaggctacgcgctgcacgacgactactacggattggacgtctggggccaaggaactcttgtgaccgtgtcctctggtggaggcggatcagggggtggcggatctgggggtggtggttccgggggaggaggatcgcagagcgcgctgactcagcctgcctccgtgagcggttcgccgggacagtccattaccatttcgtgcaccgggacctcctccgacgtgggaggctacaactacgtgtcctggtaccagcagcatcccggaaaggccccgaagctgatgatctacgaagtgtcgaacagactgcggggagtctccaaccgcttttccgggtccaagtccggcaacaccgccagcctgaccatcagcgggctccaggcagaagatgaggctgactattactgctcctcctacacgtcaagctccaccctctacgtgttcgggtccgggaccaaagtcactgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 130 - protein, 17 aa, synthetic construct
  REGION 1..17: note = Description of Artificial Sequence: Synthetic peptide
  MOD_RES 5: note = D or K
VISYXGSNKYYADSVKG

SEQ ID NO: 131 - protein, 7 aa, synthetic construct
  REGION 1..7: note = Description of Artificial Sequence: Synthetic peptide
  MOD_RES 1: note = D or E
  MOD_RES 6: note = P or L
  MOD_RES 7: note = S or R
XVSNRXX

SEQ ID NO: 132 - protein, 11 aa, synthetic construct
  REGION 1..11: note = Description of Artificial Sequence: Synthetic peptide
  MOD_RES 8: note = T or A
SSYTSSSXLYV

SEQ ID NO: 133 - protein, 6 aa, synthetic construct
  REGION 1..6: note = Description of Artificial Sequence: Synthetic peptide
  MOD_RES 3: note = D or K
SYXGSN

SEQ ID NO: 134 - skipped

SEQ ID NO: 135 - protein, 8 aa, synthetic construct
  REGION 1..8: note = Description of Artificial Sequence: Synthetic peptide
  MOD_RES 6: note = T or A
YTSSSXLY

SEQ ID NO: 136 - protein, 8 aa, synthetic construct
  REGION 1..8: note = Description of Artificial Sequence: Synthetic peptide
  MOD_RES 4: note = D or K
ISYXGSNK

SEQ ID NO: 137 - protein, 5 aa, synthetic construct
  REGION 1..5: note = Description of Artificial Sequence: Synthetic peptide
GFWMS

SEQ ID NO: 138 - protein, 17 aa, synthetic construct
  REGION 1..17: note = Description of Artificial Sequence: Synthetic peptide
NIKQDGSEKYYVDSVRG

SEQ ID NO: 139 - protein, 9 aa, synthetic construct
  REGION 1..9: note = Description of Artificial Sequence: Synthetic peptide
ALDYYGMDV

SEQ ID NO: 140 - protein, 7 aa, synthetic construct
  REGION 1..7: note = Description of Artificial Sequence: Synthetic peptide
GFTFSGF

SEQ ID NO: 141 - protein, 6 aa, synthetic construct
  REGION 1..6: note = Description of Artificial Sequence: Synthetic peptide
KQDGSE

SEQ ID NO: 142 - protein, 8 aa, synthetic construct
  REGION 1..8: note = Description of Artificial Sequence: Synthetic peptide
GFTFSGFW

SEQ ID NO: 143 - protein, 8 aa, synthetic construct
  REGION 1..8: note = Description of Artificial Sequence: Synthetic peptide
IKQDGSEK

SEQ ID NO: 144 - protein, 11 aa, synthetic construct
  REGION 1..11: note = Description of Artificial Sequence: Synthetic peptide
ARALDYYGMDV

SEQ ID NO: 145 - protein, 118 aa, synthetic construct
  REGION 1..118: note = Description of Artificial Sequence: Synthetic polypeptide
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSS

SEQ ID NO: 146 - DNA, 354 bp, synthetic construct
  misc_feature 1..354: note = Description of Artificial Sequence: Synthetic polynucleotide
  source 1..354: mol_type = other DNA
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagc

SEQ ID NO: 147 - protein, 17 aa, synthetic construct
  REGION 1..17: note = Description of Artificial Sequence: Synthetic peptide
RSSQSLLDSDDGNTYLD

SEQ ID NO: 148 - protein, 7 aa, synthetic construct
  REGION 1..7: note = Description of Artificial Sequence: Synthetic peptide
TLSYRAS

SEQ ID NO: 149 - protein, 10 aa, synthetic construct
  REGION 1..10: note = Description of Artificial Sequence: Synthetic peptide
TQRLEFPSIT

SEQ ID NO: 150 - protein, 13 aa, synthetic construct
  REGION 1..13: note = Description of Artificial Sequence: Synthetic peptide
SQSLLDSDDGNTY

SEQ ID NO: 151 - skipped

SEQ ID NO: 152 - protein, 7 aa, synthetic construct
  REGION 1..7: note = Description of Artificial Sequence: Synthetic peptide
RLEFPSI

SEQ ID NO: 153 - protein, 12 aa, synthetic construct
  REGION 1..12: note = Description of Artificial Sequence: Synthetic peptide
QSLLDSDDGNTY

SEQ ID NO: 154 - protein, 114 aa, synthetic construct
  REGION 1..114: note = Description of Artificial Sequence: Synthetic polypeptide
DIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 155 - DNA, 342 bp, synthetic construct
  misc_feature 1..342: note = Description of Artificial Sequence: Synthetic polynucleotide
  source 1..342: mol_type = other DNA
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 156 - protein, 252 aa, synthetic construct
  REGION 1..252: note = Description of Artificial Sequence: Synthetic polypeptide
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIK

SEQ ID NO: 157 - DNA, 756 bp, synthetic construct
  misc_feature 1..756: note = Description of Artificial Sequence: Synthetic polynucleotide
  source 1..756: mol_type = other DNA
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaag

SEQ ID NO: 158 - protein, 475 aa, synthetic construct
  REGION 1..475: note = Description of Artificial Sequence: Synthetic polypeptide
EVQLVESGGGLVQPGGSLRLSCAASGFTFSGFWMSWVRQAPGKGLEWVANIKQDGSEKYYVDSVRGRFTISRDNAKNSLYLQMNSLRAEDTAVYYCARALDYYGMDVWGQGTTVTVSSGGGGSGGGGSGGGGSGGGGSDIVMTQTPLSLPVTPGEPASISCRSSQSLLDSDDGNTYLDWYLQKPGQSPRLLIYTLSYRASGVPDRFSGSGSGTDFTLKISRVEAEDVGLYYCTQRLEFPSITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 159 - DNA, 1425 bp, synthetic construct
  misc_feature 1..1425: note = Description of Artificial Sequence: Synthetic polynucleotide
  source 1..1425: mol_type = other DNA
gaagtgcaactggtggagagcggtggagggcttgtccagcccggaggatcgctgcggctgtcctgtgctgcgtccgggttcaccttctccggcttctggatgtcctgggtcagacaggcaccgggaaagggcctcgaatgggtggccaacatcaagcaggatggctccgagaagtactacgtcgactccgtgagaggccgcttcaccatctcccgggacaacgccaagaactcgctgtacctccaaatgaatagcctcagggcggaagatactgctgtgtattactgcgcacgcgcccttgactactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcgcctgctgatctataccctgtcataccgggcctcaggagtgcctgaccgcttctcgggatcagggagcgggaccgatttcaccctgaaaatttcccgagtggaagccgaggacgtcggactgtactactgcacccagcgcctcgaattcccgtcgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 160 - protein, 5 aa, synthetic construct
  REGION 1..5: note = Description of Artificial Sequence: Synthetic peptide
SFRMN

SEQ ID NO: 161 - protein, 17 aa, synthetic construct
  REGION 1..17: note = Description of Artificial Sequence: Synthetic peptide
SISSSSSYIYYADSVKG

SEQ ID NO: 162 - protein, 9 aa, synthetic construct
  REGION 1..9: note = Description of Artificial Sequence: Synthetic peptide
WLSYYGMDV

SEQ ID NO: 163 - protein, 7 aa, synthetic construct
  REGION 1..7: note = Description of Artificial Sequence: Synthetic peptide
GFTFSSF

SEQ ID NO: 164 - protein, 6 aa, synthetic construct
  REGION 1..6: note = Description of Artificial Sequence: Synthetic peptide
SSSSSY

SEQ ID NO: 165 - protein, 8 aa, synthetic construct
  REGION 1..8: note = Description of Artificial Sequence: Synthetic peptide
GFTFSSFR

SEQ ID NO: 166 - protein, 8 aa, synthetic construct
  REGION 1..8: note = Description of Artificial Sequence: Synthetic peptide
ISSSSSYI

SEQ ID NO: 167 - protein, 11 aa, synthetic construct
  REGION 1..11: note = Description of Artificial Sequence: Synthetic peptide
ARWLSYYGMDV

SEQ ID NO: 168 - protein, 118 aa, synthetic construct
  REGION 1..118: note = Description of Artificial Sequence: Synthetic polypeptide
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSS

SEQ ID NO: 169 - DNA, 354 bp, synthetic construct
  misc_feature 1..354: note = Description of Artificial Sequence: Synthetic polynucleotide
  source 1..354: mol_type = other DNA
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagc

SEQ ID NO: 170 - protein, 7 aa, synthetic construct
  REGION 1..7: note = Description of Artificial Sequence: Synthetic peptide
TLSFRAS

SEQ ID NO: 171 - protein, 9 aa, synthetic construct
  REGION 1..9: note = Description of Artificial Sequence: Synthetic peptide
MQRIGFPIT

SEQ ID NO: 172 - protein, 6 aa, synthetic construct
  REGION 1..6: note = Description of Artificial Sequence: Synthetic peptide
RIGFPI

SEQ ID NO: 173 - protein, 113 aa, synthetic construct
  REGION 1..113: note = Description of Artificial Sequence: Synthetic polypeptide
DIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 174 - DNA, 339 bp, synthetic construct
  misc_feature 1..339: note = Description of Artificial Sequence: Synthetic polynucleotide
  source 1..339: mol_type = other DNA
gatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 175 - protein, 251 aa, synthetic construct
  REGION 1..251: note = Description of Artificial Sequence: Synthetic polypeptide
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIK

SEQ ID NO: 176 - DNA, 753 bp, synthetic construct
  misc_feature 1..753: note = Description of Artificial Sequence: Synthetic polynucleotide
  source 1..753: mol_type = other DNA
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaag

SEQ ID NO: 177 - protein, 474 aa, synthetic construct
  REGION 1..474: note = Description of Artificial Sequence: Synthetic polypeptide
EVQLVESGGGLVKPGGSLRLSCAASGFTFSSFRMNWVRQAPGKGLEWVSSISSSSSYIYYADSVKGRFTISRDNAKNSLYLQMNSLRAEDTAVYYCARWLSYYGMDVWGQGTTVTVSSGGGGSGGGGSGGGGSGGGGSDIVMTQTPLSLPVTPGEPASISCRSSQSLLDSDDGNTYLDWYLQKPGQSPQLLIYTLSFRASGVPDRFSGSGSGTDFTLKIRRVEAEDVGVYYCMQRIGFPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 178 - DNA, 1422 bp, synthetic construct
  misc_feature 1..1422: note = Description of Artificial Sequence: Synthetic polynucleotide
  source 1..1422: mol_type = other DNA
gaagtgcaactggtggagagcggtggagggcttgtcaagcccggaggatcgctgcggctgtcctgtgctgcgtccgggttcaccttctcctcgttccgcatgaactgggtcagacaggcaccgggaaagggcctcgaatgggtgtcctcaatctcatcgtcctcgtcctacatctactacgccgactccgtgaaaggccgcttcaccatctcccgggacaacgccaagaactcgctgtacctccaaatgaatagcctcagggcggaagatactgctgtgtattactgcgcacgctggctttcctactacggcatggacgtctggggccaagggaccactgtgaccgtgtctagcggaggcggaggttcagggggcggtggatcaggcggaggaggatcggggggtggtggatcggatatcgtgatgacccagactcccctgtccctgcctgtgactcccggagaaccagcctccatttcctgccggtcctcccagtccctgctggacagcgacgacggcaacacttacctggactggtacttgcagaagccgggccaatcgcctcagctgctgatctataccctgtcattccgggcctcaggagtgcctgaccgcttctcgggatcagggagcgggaccgatttcaccctgaaaattaggcgagtggaagccgaggacgtcggagtgtactactgcatgcagcgcatcggcttcccgattacgtttggacagggtacccggcttgagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 179 - skipped

SEQ ID NO: 180 - protein, 17 aa, synthetic construct
  REGION 1..17: note = Description of Artificial Sequence: Synthetic peptide
  MOD_RES 1: note = N or S
  MOD_RES 3: note = K or S
  MOD_RES 4: note = Q or S
  MOD_RES 5: note = D or S
  MOD_RES 6: note = G or S
  MOD_RES 8: note = E or Y
  MOD_RES 9: note = K or I
  MOD_RES 12: note = V or A
  MOD_RES 16: note = R or K
XIXXXXSXXYYXDSVXG

SEQ ID NO: 181 - protein, 9 aa, synthetic construct
  REGION 1..9: note = Description of Artificial Sequence: Synthetic peptide
  MOD_RES 1: note = A or W
  MOD_RES 3: note = D or S
XLXYYGMDV

SEQ ID NO: 182 - protein, 7 aa, synthetic construct
  REGION 1..7: note = Description of Artificial Sequence: Synthetic peptide
  MOD_RES 4: note = Y or F
TLSXRAS

SEQ ID NO: 183 - protein, 10 aa, synthetic construct
  REGION 1..10: note = Description of Artificial Sequence: Synthetic peptide
  MOD_RES 1: note = T or M
  MOD_RES 4: note = L or I
  MOD_RES 5: note = E or G
  MOD_RES 8: note = S or absent
XQRXXFPXIT

SEQ ID NO: 184 - protein, 7 aa, synthetic construct
  REGION 1..7: note = Description of Artificial Sequence: Synthetic peptide
  MOD_RES 6: note = G or S
GFTFSXF

SEQ ID NO: 185 - skipped

SEQ ID NO: 186 - protein, 7 aa, synthetic construct
  REGION 1..7: note = Description of Artificial Sequence: Synthetic peptide
  MOD_RES 2: note = L or I
  MOD_RES 3: note = E or G
  MOD_RES 6: note = S or absent
RXXFPXI

SEQ ID NO: 187 - protein, 8 aa, synthetic construct
  REGION 1..8: note = Description of Artificial Sequence: Synthetic peptide
  MOD_RES 6: note = G or S
  MOD_RES 8: note = W or R
GFTFSXFX

SEQ ID NO: 188 - skipped

SEQ ID NO: 189 - protein, 11 aa, synthetic construct
  REGION 1..11: note = Description of Artificial Sequence: Synthetic peptide
  MOD_RES 3: note = A or W
  MOD_RES 5: note = D or S
ARXLXYYGMDV

SEQ ID NO: 190 - DNA, 521 bp, unidentified
  misc_feature 1..521: note = Description of Unknown: PGK Promoter sequence
  source 1..521: mol_type = unassigned DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccgcggcgacgcaaagggccttggtgcgggtctcgtcggcgcagggacgcgtttgggtcccgacggaaccttttccgcgttggggttggggcaccataagct

SEQ ID NO: 191 - DNA, 221 bp, synthetic construct
  misc_feature 1..221: note = Description of Artificial Sequence: Synthetic polynucleotide
  source 1..221: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacg

SEQ ID NO: 192 - DNA, 324 bp, synthetic construct
  misc_feature 1..324: note = Description of Artificial Sequence: Synthetic polynucleotide
  source 1..324: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccg

SEQ ID NO: 193 - DNA, 422 bp, synthetic construct
  misc_feature 1..422: note = Description of Artificial Sequence: Synthetic polynucleotide
  source 1..422: mol_type = other DNA
acccctctctccagccactaagccagttgctccctcggctgacggctgcacgcgaggcctccgaacgtcttacgccttgtggcgcgcccgtccttgtcccgggtgtgatggcggggtgtggggcggagggcgtggcggggaagggccggcgacgagagccgcgcgggacgactcgtcggcgataaccggtgtcgggtagcgccagccgcgcgacggtaacgagggaccgcgacaggcagacgctcccatgatcactctgcacgccgaaggcaaatagtgcaggccgtgcggcgcttggcgttccttggaagggctgaatccccgcctcgtccttcgcagcggccccccgggtgttcccatcgccgcttctaggcccactgcgacgcttgcctgcacttcttacacgctctgggtcccagccg

SEQ ID NO: 194-197 - skipped (4 entries)

SEQ ID NO: 198 - DNA, 118 bp, synthetic construct
  misc_feature 1..118: note = Description of Artificial Sequence: Synthetic polynucleotide
  source 1..118: mol_type = other DNA
acccctctctccagccactaagccagttgctccctcggctgacggctgcacgcgaggcctccgaacgtcttacgccttgtggcgcgcccgtccttgtcccgggtgtgatggcggggtg

SEQ ID NO: 199 - DNA, 63 bp, synthetic construct
  misc_feature 1..63: note = Description of Artificial Sequence: Synthetic oligonucleotide
  source 1..63: mol_type = other DNA
atggccctccctgtcaccgccctgctgcttccgctggctcttctgctccacgccgctcggccc

SEQ ID NO: 200 - protein, 239 aa, synthetic construct
  REGION 1..239: note = Description of Artificial Sequence: Synthetic polypeptide
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 201 - DNA, 717 bp, synthetic construct
  misc_feature 1..717: note = Description of Artificial Sequence: Synthetic polynucleotide
  source 1..717: mol_type = other DNA
gaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcgcgtccggcggaggcggcagcgggggtggtggttcagggggcggcggatcggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaag

SEQ ID NO: 202 - protein, 69 aa, synthetic construct
  REGION 1..69: note = Description of Artificial Sequence: Synthetic polypeptide
TTTPAPRPPTPAPTIASQPLSLRPEACRPAAGGAVHTRGLDFACDIYIWAPLAGTCGVLLLSLVITLYC

SEQ ID NO: 203 - DNA, 207 bp, synthetic construct
  misc_feature 1..207: note = Description of Artificial Sequence: Synthetic polynucleotide
  source 1..207: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgt

SEQ ID NO: 204 - DNA, 126 bp, synthetic construct
  misc_feature 1..126: note = Description of Artificial Sequence: Synthetic polynucleotide
  source 1..126: mol_type = other DNA
aagcgcggtcggaagaagctgctgtacatctttaagcaacccttcatgaggcctgtgcagactactcaagaggaggacggctgttcatgccggttcccagaggaggaggaaggcggctgcgaactg

SEQ ID NO: 205 - DNA, 336 bp, synthetic construct
  misc_feature 1..336: note = Description of Artificial Sequence: Synthetic polynucleotide
  source 1..336: mol_type = other DNA
cgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 206-207 - skipped (2 entries)

SEQ ID NO: 208 - protein, 19 aa, synthetic construct
  REGION 1..19: note = Description of Artificial Sequence: Synthetic peptide
ATNFSLLKQAGDVEENPGP

SEQ ID NO: 209 - DNA, 57 bp, synthetic construct
  misc_feature 1..57: note = Description of Artificial Sequence: Synthetic oligonucleotide
  source 1..57: mol_type = other DNA
gctactaacttcagcctgctgaagcaggctggagacgtggaggagaaccctggacct

SEQ ID NO: 210 - DNA, 63 bp, synthetic construct
  misc_feature 1..63: note = Description of Artificial Sequence: Synthetic oligonucleotide
  source 1..63: mol_type = other DNA
atggccttaccagtgaccgccttgctcctgccgctggccttgctgctccacgccgccaggccg

SEQ ID NO: 211 - protein, 242 aa, synthetic construct
  REGION 1..242: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 212 - DNA, 726 bp, synthetic construct
  misc_feature 1..726: note = Description of Artificial Sequence: Synthetic polynucleotide
  source 1..726: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 213 - DNA, 207 bp, synthetic construct
  misc_feature 1..207: note = Description of Artificial Sequence: Synthetic polynucleotide
  source 1..207: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgc

SEQ ID NO: 214 - protein, 1000 aa, synthetic construct
  REGION 1..1000: note = Description of Artificial Sequence: Synthetic polypeptide
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 215 - DNA, 3000 bp, synthetic construct
  misc_feature 1..3000: note = Description of Artificial Sequence: Synthetic polynucleotide
  source 1..3000: mol_type = other DNA
atggccctccctgtcaccgccctgctgcttccgctggctcttctgctccacgccgctcggcccgaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtggtggggagaaagctggctgttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggaagcggagctactaacttcagcctgctgaagcaggctggagacgtggaggagaaccctggacctatggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 216 - protein, 1001 aa, synthetic construct
  REGION 1..1001: note = Description of Artificial Sequence: Synthetic polypeptide
MALPVTALLLPLALLLHAARPQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 217 - DNA, 3003 bp, synthetic construct
  misc_feature 1..3003: note = Description of Artificial Sequence: Synthetic polynucleotide
  source 1..3003: mol_type = other DNA
atggccctccctgtcaccgccctgctgcttccgctggctcttctgctccacgccgctcggccccaagtgcagctgcaggaatccggtggcggagtcgtgcagcctggaaggagcctgagactctcatgcgccgcgtcagggttcaccttttcctcctacgggatgcattgggtcagacaggcccccggaaagggactcgaatgggtggctgtgatcagctacgacggctccaacaagtactacgccgactccgtgaaaggccggttcactatctcccgggacaactccaagaacacgctgtatctgcaaatgaattcactgcgcgcggaggataccgctgtgtactactgcggtggctccggttacgccctgcacgatgactattacggccttgacgtctggggccagggaaccctcgtgactgtgtccagcggtggaggaggttcgggcggaggaggatcaggagggggtggatcgcagagcgcactgactcagccggcatccgtgtccggtagccccggacagtcgattaccatctcctgtaccggcacctcctccgacgtgggagggtacaactacgtgtcgtggtaccagcagcacccaggaaaggcccctaagttgatgatctacgatgtgtcaaaccgcccgtctggagtctccaaccggttctccggctccaagtccggcaacaccgccagcctgaccattagcgggctgcaagccgaggatgaggccgactactactgctcgagctacacatcctcgagcaccctctacgtgttcggctcggggactaaggtcaccgtgctgaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggaagcggagctactaacttcagcctgctgaagcaggctggagacgtggaggagaaccctggacctatggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 218 - protein, 1002 aa, synthetic construct
  REGION 1..1002: note = Description of Artificial Sequence: Synthetic polypeptide
MALPVTALLLPLALLLHAARPEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 219 - DNA, 3006 bp, synthetic construct
  misc_feature 1..3006: note = Description of Artificial Sequence: Synthetic polynucleotide
  source 1..3006: mol_type = other DNA
atggccctccctgtcaccgccctgctgcttccgctggctcttctgctccacgccgctcggcccgaagtgcagttgctggagtcaggcggaggactggtgcagcccggaggatcgcttcgcttgagctgcgcagcctcaggctttaccttctcctcctacgccatgtcctgggtcagacaggctcccgggaagggactggaatgggtgtccgccattagcggttccggcggaagcacttactatgccgactctgtgaagggccgcttcactatctcccgggacaactccaagaacaccctgtatctccaaatgaattccctgagggccgaagataccgcggtgtactactgcgctagacgggagtgggtgccctacgatgtcagctggtacttcgactactggggacagggcactctcgtgactgtgtcctccggtggtggtggatcggggggtggtggttcgggcggaggaggatctggaggaggagggtcggacattcaaatgactcagtccccgtcctccctctccgcctccgtgggagatcgcgtcacgatcacgtgcagggccagccagagcatctccagctacctgaactggtaccagcagaagccagggaaggcaccgaagctcctgatctacgccgctagctcgctgcagtccggcgtcccttcacggttctcgggatcgggctcaggcaccgacttcaccctgaccattagcagcctgcagccggaggacttcgcgacatactactgtcagcagtcatactccacccctctgaccttcggccaagggaccaaagtggagatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggaagcggagctactaacttcagcctgctgaagcaggctggagacgtggaggagaaccctggacctatggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 220 - protein, 991 aa, synthetic construct
  REGION 1..991: note = Description of Artificial Sequence: Synthetic polypeptide
MALPVTALLLPLALLLHAARPEVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 221 - DNA, 2973 bp, synthetic construct
  misc_feature 1..2973: note = Description of Artificial Sequence: Synthetic polynucleotide
  source 1..2973: mol_type = other DNA
atggccctccctgtcaccgccctgctgcttccgctggctcttctgctccacgccgctcggcccgaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcgcgtccggcggaggcggcagcgggggtggtggttcagggggcggcggatcggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggggaagcggagctactaacttcagcctgctgaagcaggctggagacgtggaggagaaccctggacctatggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 222 - protein, 991 aa, synthetic construct
  REGION 1..991: note = Description of Artificial Sequence: Synthetic polypeptide
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPMALPVTALLLPLALLLHAARPEVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 223 - DNA, 2973 bp, synthetic construct
  misc_feature 1..2973: note = Description of Artificial Sequence: Synthetic polynucleotide
  source 1..2973: mol_type = other DNA
atggccttaccagtgaccgccttgctcctgccgctggccttgctgctccacgccgccaggccggaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaagcggagctactaacttcagcctgctgaagcaggctggagacgtggaggagaaccctggacctatggccctccctgtcaccgccctgctgcttccgctggctcttctgctccacgccgctcggcccgaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagcgcgtccggcggaggcggcagcgggggtggtggttcagggggcggcggatcggacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaagaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 224 - protein, 492 aa, synthetic construct
  REGION 1..492: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 225 - protein, 465 aa, synthetic construct
  REGION 1..465: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 226 - protein, 493 aa, synthetic construct
  REGION 1..493: note = Description of Artificial Sequence: Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 227 - protein, 494 aa, synthetic construct
  REGION 1..494: note = Description of Artificial Sequence: Synthetic polypeptide
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWVPYDVSWYFDYWGQGTLVTVSSGGGGSGGGGSGGGGSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 228 - protein, 483 aa, synthetic construct
  REGION 1..483: note = Description of Artificial Sequence: Synthetic polypeptide
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 229 - protein, 486 aa, synthetic construct
  REGION 1..486: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPG

SEQ ID NO: 230 - protein, 462 aa, synthetic construct
  REGION 1..462: note = Description of Artificial Sequence: Synthetic polypeptide
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSSASGGGGSGGGGSGGGGSDIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 231 - protein, 5 aa, synthetic construct
  REGION 1..5: note = Description of Artificial Sequence: Synthetic peptide
NHGMS

SEQ ID NO: 232 - protein, 16 aa, synthetic construct
  REGION 1..16: note = Description of Artificial Sequence: Synthetic peptide
GIVYSGSTYYAASVKG

SEQ ID NO: 233 - protein, 7 aa, synthetic construct
  REGION 1..7: note = Description of Artificial Sequence: Synthetic peptide
HGGESDV

SEQ ID NO: 234 - protein, 7 aa, synthetic construct
  REGION 1..7: note = Description of Artificial Sequence: Synthetic peptide
GFALSNH

SEQ ID NO: 235 - protein, 5 aa, synthetic construct
  REGION 1..5: note = Description of Artificial Sequence: Synthetic peptide
VYSGS

SEQ ID NO: 236 - protein, 8 aa, synthetic construct
  REGION 1..8: note = Description of Artificial Sequence: Synthetic peptide
GFALSNHG

SEQ ID NO: 237 - protein, 7 aa, synthetic construct
  REGION 1..7: note = Description of Artificial Sequence: Synthetic peptide
IVYSGST

SEQ ID NO: 238 - protein, 9 aa, synthetic construct
  REGION 1..9: note = Description of Artificial Sequence: Synthetic peptide
SAHGGESDV

SEQ ID NO: 239 - protein, 115 aa, synthetic construct
  REGION 1..115: note = Description of Artificial Sequence: Synthetic polypeptide
EVQLVESGGGLVQPGGSLRLSCAVSGFALSNHGMSWVRRAPGKGLEWVSGIVYSGSTYYAASVKGRFTISRDNSRNTLYLQMNSLRPEDTAIYYCSAHGGESDVWGQGTTVTVSS

SEQ ID NO: 240 - protein, 9 aa, synthetic construct
  REGION 1..9: note = Description of Artificial Sequence: Synthetic peptide
QQSYSTPYT

SEQ ID NO: 241 - protein, 6 aa, synthetic construct
  REGION 1..6: note = Description of Artificial Sequence: Synthetic peptide
SYSTPY

SEQ ID NO: 242 - protein, 107 aa, synthetic construct
  REGION 1..107: note = Description of Artificial Sequence: Synthetic polypeptide
DIQLTQSPSSLSASVGDRVTITCRASQSISSYLNWYQQKPGKAPKLLIYAASSLQSGVPSRFSGSGSGTDFTLTISSLQPEDFATYYCQQSYSTPYTFGQGTKVEIK

SEQ ID NO: 243 - protein, 17 aa, synthetic construct
  REGION 1..17: note = Description of Artificial Sequence: Synthetic peptide
ASGGGGSGGGGSGGGGS

SEQ ID NO: 244 - protein, 10 aa, synthetic construct
  REGION 1..10: note = Description of Artificial Sequence: Synthetic peptide
GVSLPDYGVS

SEQ ID NO: 245 - protein, 16 aa, synthetic construct
  REGION 1..16: note = Description of Artificial Sequence: Synthetic peptide
VIWGSETTYYQSSLKS

SEQ ID NO: 246 - protein, 12 aa, synthetic construct
  REGION 1..12: note = Description of Artificial Sequence: Synthetic peptide
HYYYGGSYAMDY

SEQ ID NO: 247 - protein, 11 aa, synthetic construct
  REGION 1..11: note = Description of Artificial Sequence: Synthetic peptide
RASQDISKYLN

SEQ ID NO: 248 - protein, 7 aa, synthetic construct
  REGION 1..7: note = Description of Artificial Sequence: Synthetic peptide
HTSRLHS

SEQ ID NO: 249 - protein, 9 aa, synthetic construct
  REGION 1..9: note = Description of Artificial Sequence: Synthetic peptide
QQGNTLPYT

SEQ ID NO: 250 - protein, 120 aa, synthetic construct
  REGION 1..120: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 251 - protein, 107 aa, synthetic construct
  REGION 1..107: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 252 - DNA, 63 bp, synthetic construct
  misc_feature 1..63: note = Description of Artificial Sequence: Synthetic oligonucleotide
  source 1..63: mol_type = other DNA
atggccctccctgtcaccgctctgttgctgccgcttgctctgctgctccacgcagcgcgaccg

SEQ ID NO: 253 - DNA, 747 bp, synthetic construct
  misc_feature 1..747: note = Description of Artificial Sequence: Synthetic polynucleotide
  source 1..747: mol_type = other DNA
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactc

SEQ ID NO: 254 - DNA, 207 bp, synthetic construct
  misc_feature 1..207: note = Description of Artificial Sequence: Synthetic polynucleotide
  source 1..207: mol_type = other DNA
acaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgt

SEQ ID NO: 255 - DNA, 126 bp, synthetic construct
  misc_feature 1..126: note = Description of Artificial Sequence: Synthetic polynucleotide
  source 1..126: mol_type = other DNA
aaacgcgggcgaaaaaaattgctgtatatttttaagcagccatttatgaggcccgttcagacgacgcaggaggaggacggttgctcttgcaggttcccagaagaggaagaagggggctgtgaattg

SEQ ID NO: 256 - DNA, 336 bp, synthetic construct
  misc_feature 1..336: note = Description of Artificial Sequence: Synthetic polynucleotide
  source 1..336: mol_type = other DNA
cgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 257 - protein, 493 aa, synthetic construct
  REGION 1..493: note = Description of Artificial Sequence: Synthetic polypeptide
MALPVTALLLPLALLLHAARPQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 258 - DNA, 1479 bp, synthetic construct
  misc_feature 1..1479: note = Description of Artificial Sequence: Synthetic polynucleotide
  source 1..1479: mol_type = other DNA
atggccctccctgtcaccgctctgttgctgccgcttgctctgctgctccacgcagcgcgaccgcaggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 259 - DNA, 1416 bp, synthetic construct
  misc_feature 1..1416: note = Description of Artificial Sequence: Synthetic polynucleotide
  source 1..1416: mol_type = other DNA
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgaga

SEQ ID NO: 260 - DNA, 369 bp, synthetic construct
  misc_feature 1..369: note = Description of Artificial Sequence: Synthetic polynucleotide
  source 1..369: mol_type = other DNA
caggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctct

SEQ ID NO: 261 - DNA, 333 bp, synthetic construct
  misc_feature 1..333: note = Description of Artificial Sequence: Synthetic polynucleotide
  source 1..333: mol_type = other DNA
caatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactc

SEQ ID NO: 262 - DNA, 345 bp, synthetic construct
  misc_feature 1..345: note = Description of Artificial Sequence: Synthetic polynucleotide
  source 1..345: mol_type = other DNA
gaagtgcaattggtggaatcagggggaggacttgtgcagcctggaggatcgctgagactgtcatgtgccgtgtccggctttgccctgtccaaccacgggatgtcctgggtccgccgcgcgcctggaaagggcctcgaatgggtgtcgggtattgtgtacagcggtagcacctactatgccgcatccgtgaaggggagattcaccatcagccgggacaactccaggaacactctgtacctccaaatgaattcgctgaggccagaggacactgccatctactactgctccgcgcatggcggagagtccgacgtctggggacaggggaccaccgtgaccgtgtctagc

SEQ ID NO: 263 - DNA, 321 bp, synthetic construct
  misc_feature 1..321: note = Description of Artificial Sequence: Synthetic polynucleotide
  source 1..321: mol_type = other DNA
gacatccagctcacccagtccccgagctcgctgtccgcctccgtgggagatcgggtcaccatcacgtgccgcgccagccagtcgatttcctcctacctgaactggtaccaacagaagcccggaaaagccccgaagcttctcatctacgccgcctcgagcctgcagtcaggagtgccctcacggttctccggctccggttccggtactgatttcaccctgaccatttcctccctgcaaccggaggacttcgctacttactactgccagcagtcgtactccaccccctacactttcggacaaggcaccaaggtcgaaatcaag

SEQ ID NO: 264-274 - skipped (11 entries)

SEQ ID NO: 275 - protein, 132 aa, synthetic construct
  REGION 1..132: note = Description of Artificial Sequence: Synthetic polypeptide
DVPDYASLGGPSSPKKKRKVSRGVQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETSY

SEQ ID NO: 276 - protein, 108 aa, synthetic construct
  REGION 1..108: note = Description of Artificial Sequence: Synthetic polypeptide
VQVETISPGDGRTFPKRGQTCVVHYTGMLEDGKKFDSSRDRNKPFKFMLGKQEVIRGWEEGVAQMSVGQRAKLTISPDYAYGATGHPGIIPPHATLVFDVELLKLETS

SEQ ID NO: 277 - protein, 93 aa, synthetic construct [sequence omitted]

SEQ ID NO: 278 - protein, 95 aa, synthetic construct
  REGION 1..95: note = Description of Artificial Sequence: Synthetic polypeptide
ILWHEMWHEGLIEASRLYFGERNVKGMFEVLEPLHAMMERGPQTLKETSFNQAYGRDLMEAQEWCRKYMKSGNVKDLTQAWDLYYHVFRRISKTS

SEQ ID NO: 279 - protein, 95 aa, synthetic construct
  REGION 1..95: note = Description of Artificial Sequence: Synthetic polypeptide
ILWHEMWHEGLLEASRLYFGERNVKGMFEVLEPLHAMMERGPQTLKETSFNQAYGRDLMEAQEWCRKYMKSGNVKDLTQAWDLYYHVFRRISKTS

SEQ ID NO: 280 - protein, 95 aa, synthetic construct
  REGION 1..95: note = Description of Artificial Sequence: Synthetic polypeptide
ILWHEMWHEGLEEASRLYFGERNVKGMFEVLEPLHAMMERGPQTLKETSFNQAYGRDLMEAQEWCRKYMKSGNVKDLLQAWDLYYHVFRRISKTS

SEQ ID NO: 281 - protein, 95 aa, synthetic construct
  REGION 1..95: note = Description of Artificial Sequence: Synthetic polypeptide
  MOD_RES 12: note = Any amino acid
  MOD_RES 78: note = Any amino acid
ILWHEMWHEGLXEASRLYFGERNVKGMFEVLEPLHAMMERGPQTLKETSFNQAYGRDLMEAQEWCRKYMKSGNVKDLXQAWDLYYHVFRRISKTS

SEQ ID NO: 282 - protein, 95 aa, synthetic construct
  REGION 1..95: note = Description of Artificial Sequence: Synthetic polypeptide
ILWHEMWHEGLIEASRLYFGERNVKGMFEVLEPLHAMMERGPQTLKETSFNQAYGRDLMEAQEWCRKYMKSGNVKDLLQAWDLYYHVFRRISKTS

SEQ ID NO: 283 - protein, 95 aa, synthetic construct
  REGION 1..95: note = Description of Artificial Sequence: Synthetic polypeptide
ILWHEMWHEGLLEASRLYFGERNVKGMFEVLEPLHAMMERGPQTLKETSFNQAYGRDLMEAQEWCRKYMKSGNVKDLLQAWDLYYHVFRRISKTS

SEQ ID NO: 284 - protein, 1132 aa, Homo sapiens [sequence omitted]

SEQ ID NO: 285 - protein, 253 aa, synthetic construct
  REGION 1..253: note = Description of Artificial Sequence: Synthetic polypeptide
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSSGGGGSGGGGSGGGGSQSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 286 - protein, 127 aa, synthetic construct
  REGION 1..127: note = Description of Artificial Sequence: Synthetic polypeptide
EVQLQQSGPGLVKPSQTLSLTCAISGDSMLSNSDTWNWIRQSPSRGLEWLGRTYHRSTWYDDYASSVRGRVSINVDTSKNQYSLQLNAVTPEDTGVYYCARVRLQDGNSWSDAFDVWGQGTMVTVSS

SEQ ID NO: 287 - protein, 111 aa, synthetic construct
  REGION 1..111: note = Description of Artificial Sequence: Synthetic polypeptide
QSALTQPASASGSPGQSVTISCTGTSSDVGGYNYVSWYQQHPGKAPKLMIYDVSNRPSGVSNRFSGSKSGNTASLTISGLQAEDEADYYCSSYTSSSTLYVFGTGTQLTVL

SEQ ID NO: 288 - protein, 12 aa, synthetic construct
  REGION 1..12: note = Description of Artificial Sequence: Synthetic peptide
GDSMLSNSDTWN

SEQ ID NO: 289 - protein, 7 aa, synthetic construct
  REGION 1..7: note = Description of Artificial Sequence: Synthetic peptide
SNSDTWN

SEQ ID NO: 290 - protein, 18 aa, synthetic construct
  REGION 1..18: note = Description of Artificial Sequence: Synthetic peptide
RTYHRSTWYDDYASSVRG

SEQ ID NO: 291 - protein, 15 aa, synthetic construct
  REGION 1..15: note = Description of Artificial Sequence: Synthetic peptide
VRLQDGNSWSDAFDV

SEQ ID NO: 292 - protein, 465 aa, synthetic construct
  REGION 1..465: note = Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 293 - protein, 242 aa, synthetic construct; identical to SEQ ID NO: 211
  REGION 1..242: note = Description of Artificial Sequence: Synthetic polypeptide

SEQ ID NO: 294 - protein, 465 aa, synthetic construct
  REGION 1..465: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 295 - protein, 5 aa, synthetic construct
  REGION 1..5: note = Description of Artificial Sequence: Synthetic peptide
DYGVS

SEQ ID NO: 296 - protein, 16 aa, synthetic construct
  REGION 1..16: note = Description of Artificial Sequence: Synthetic peptide
VIWGSETTYYNSALKS

SEQ ID NO: 297 - protein, 12 aa, synthetic construct
  REGION 1..12: note = Description of Artificial Sequence: Synthetic peptide
HYYYGGSYAMDY

SEQ ID NO: 298 - protein, 11 aa, synthetic construct
  REGION 1..11: note = Description of Artificial Sequence: Synthetic peptide
RASQDISKYLN

SEQ ID NO: 299 - protein, 7 aa, synthetic construct
  REGION 1..7: note = Description of Artificial Sequence: Synthetic peptide
HTSRLHS

SEQ ID NO: 300 - protein, 9 aa, synthetic construct
  REGION 1..9: note = Description of Artificial Sequence: Synthetic peptide
QQGNTLPYT

SEQ ID NO: 301 - protein, 486 aa, synthetic construct
  REGION 1..486: note = Description of Artificial Sequence: Synthetic polypeptide
MALPVTALLLPLALLLHAARPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 302 - DNA, 1458 bp, synthetic construct
  misc_feature 1..1458: note = Description of Artificial Sequence: Synthetic polynucleotide
  source 1..1458: mol_type = other DNA
atggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaaggaacctcagtcaccgtctcctcaaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 303 - protein, 242 aa, synthetic construct
  REGION 1..242: note = Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 304 - protein, 16 aa, synthetic construct
  REGION 1..16: note = Description of Artificial Sequence: Synthetic peptide
VIWGSETTYYQSSLKS

SEQ ID NO: 305 - DNA, 726 bp, synthetic construct; identical to SEQ ID NO: 212
  misc_feature 1..726: note = Description of Artificial Sequence: Synthetic polynucleotide
  source 1..726: mol_type = other DNA

SEQ ID NO: 306 - protein, 486 aa, synthetic construct
  REGION 1..486: note = Description of Artificial Sequence: Synthetic polypeptide
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 307 - DNA, 1458 bp, synthetic construct
  misc_feature 1..1458: note = Description of Artificial Sequence: Synthetic polynucleotide
  source 1..1458: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctacaagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 308 - protein, 7 aa, synthetic construct
  REGION 1..7: note = Description of Artificial Sequence: Synthetic peptide
LAEAAAK

SEQ ID NO: 309 - protein, 284 aa, synthetic construct
  REGION 1..284: note = Description of Artificial Sequence: Synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQG

SEQ ID NO: 310 - protein, 7 aa, synthetic construct
  REGION 1..7: note = Description of Artificial Sequence: Synthetic peptide
GVSLPDY

SEQ ID NO: 311 - protein, 5 aa, synthetic construct
  REGION 1..5: note = Description of Artificial Sequence: Synthetic peptide
WGSET

SEQ ID NO: 312 - protein, 8 aa, synthetic construct
  REGION 1..8: note = Description of Artificial Sequence: Synthetic peptide
GVSLPDYG

SEQ ID NO: 313 - protein, 7 aa, synthetic construct
  REGION 1..7: note = Description of Artificial Sequence: Synthetic peptide
IWGSETT

SEQ ID NO: 314 - protein, 14 aa, synthetic construct
  REGION 1..14: note = Description of Artificial Sequence: Synthetic peptide
AKHYYYGGSYAMDY

SEQ ID NO: 315 - DNA, 360 bp, synthetic construct
  misc_feature 1..360: note = Description of Artificial Sequence: Synthetic polynucleotide
  source 1..360: mol_type = other DNA
caggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagc

SEQ ID NO: 316 - protein, 7 aa, synthetic construct
  REGION 1..7: note = Description of Artificial Sequence: Synthetic peptide
SQDISKY

SEQ ID NO: 317 - skipped

SEQ ID NO: 318 - protein, 6 aa, synthetic construct
  REGION 1..6: note = Description of Artificial Sequence: Synthetic peptide
GNTLPY

SEQ ID NO: 319 - protein, 6 aa, synthetic construct
  REGION 1..6: note = Description of Artificial Sequence: Synthetic peptide
QDISKY

SEQ ID NO: 320 - DNA, 321 bp, synthetic construct
  misc_feature 1..321: note = Description of Artificial Sequence: Synthetic polynucleotide
  source 1..321: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaa

SEQ ID NO: 321 - protein, 491 aa, synthetic construct
  REGION 1..491: note = Description of Artificial Sequence: Synthetic polypeptide
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSGGGGSGGGGSGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIK

SEQ ID NO: 322 - protein, 491 aa, synthetic construct
  REGION 1..491: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIKGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSQVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSS

SEQ ID NO: 323 - protein, 491 aa, synthetic construct
  REGION 1..491: note = Description of Artificial Sequence: Synthetic polypeptide
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIK

SEQ ID NO: 324 - protein, 491 aa, synthetic construct
  REGION 1..491: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIKGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSGGGGSGGGGSGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSQVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSS

SEQ ID NO: 325 - protein, 491 aa, synthetic construct
  REGION 1..491: note = Description of Artificial Sequence: Synthetic polypeptide
QVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVL

SEQ ID NO: 326 - protein, 491 aa, synthetic construct
  REGION 1..491: note = Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVLGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSQVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSS

SEQ ID NO: 327 - protein, 491 aa, synthetic construct
  REGION 1..491: note = Description of Artificial Sequence: Synthetic polypeptide
QVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVL

SEQ ID NO: 328 - protein, 491 aa, synthetic construct
  REGION 1..491: note = Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVLGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSQVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSS

SEQ ID NO: 329 - protein, 485 aa, synthetic construct
  REGION 1..485: note = Description of Artificial Sequence: Synthetic polypeptide
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIK

SEQ ID NO: 330 - protein, 485 aa, synthetic construct
  REGION 1..485: note = Description of Artificial Sequence: Synthetic polypeptide
EVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARREWWGESWLFDYWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGCGTKVEIK

SEQ ID NO: 331 - protein, 120 aa, synthetic construct
  REGION 1..120: note = Description of Artificial Sequence: Synthetic polypeptide
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSS

SEQ ID NO: 332 - protein, 107 aa, synthetic construct
  REGION 1..107: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIK

SEQ ID NO: 333 - protein, 123 aa, synthetic construct
  REGION 1..123: note = Description of Artificial Sequence: Synthetic polypeptide
QVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSS

SEQ ID NO: 334 - protein, 111 aa, synthetic construct
  REGION 1..111: note = Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVL

SEQ ID NO: 335 - protein, 121 aa, synthetic construct
  REGION 1..121: note = Description of Artificial Sequence: Synthetic polypeptide
EVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARREWWGESWLFDYWGQGTLVTVSS

SEQ ID NO: 336 - protein, 107 aa, synthetic construct
  REGION 1..107: note = Description of Artificial Sequence: Synthetic polypeptide
DIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGCGTKVEIK

SEQ ID NO: 337 - DNA, 135 bp, synthetic construct
  misc_feature 1..135: note = Description of Artificial Sequence: Synthetic polynucleotide
  source 1..135: mol_type = other DNA
accactaccccagcaccgaggccacccaccccggctcctaccatcgcctcccagcctctgtccctgcgtccggaggcatgtagacccgcagctggtggggccgtgcatacccggggtcttgacttcgcctgcgat

SEQ ID NO: 338 - DNA, 72 bp, synthetic construct
  misc_feature 1..72: note = Description of Artificial Sequence: Synthetic oligonucleotide
  source 1..72: mol_type = other DNA
atctacatttgggcccctctggctggtacttgcggggtcctgctgctttcactcgtgatcactctttactgt

SEQ ID NO: 339 - protein, 714 aa, synthetic construct
  REGION 1..714: note = Description of Artificial Sequence: Synthetic polypeptide
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSGGGGSGGGGSGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 340 - protein, 714 aa, synthetic construct
  REGION 1..714: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIKGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSQVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 341 - protein, 714 aa, synthetic construct
  REGION 1..714: note = Description of Artificial Sequence: Synthetic polypeptide
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSGGGGSGGGGSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 342 - protein, 714 aa, synthetic construct
  REGION 1..714: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIKGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGSGTKVTVLGGGGSGGGGSGGGGSGGGGSQVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCGGSGYALHDDYYGLDVWGQGTLVTVSSGGGGSQVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 343 - protein, 714 aa, synthetic construct
  REGION 1..714: note = Description of Artificial Sequence: Synthetic polypeptide
QVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 344 - protein, 714 aa, synthetic construct
  REGION 1..714: note = Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVLGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSQVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 345 - protein, 714 aa, synthetic construct
  REGION 1..714: note = Description of Artificial Sequence: Synthetic polypeptide
QVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSQSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVLTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 346 - protein, 714 aa, synthetic construct
  REGION 1..714: note = Description of Artificial Sequence: Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTLYVFGCGTKVTVLGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSQVQLQESGGGVVQPGRSLRLSCAASGFTFSSYGMHWVRQAPGKCLEWVAVISYDGSNKYYADSVKGRFTISRDNSKNTLYLQMNSLRAEDTAVYYCGGSGYALHDDYYGLDVWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 347 - protein, 708 aa, synthetic construct
  REGION 1..708: note = Description of Artificial Sequence: Synthetic polypeptide
QVQLQESGPGLVKPSETLSLTCTVSGVSLPDYGVSWIRQPPGKCLEWIGVIWGSETTYYQSSLKSRVTISKDNSKNQVSLKLSSVTAADTAVYYCAKHYYYGGSYAMDYWGQGTLVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGQGTKVEIKGGGGSGGGGSGGGGSGGGGSEVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARREWWGESWLFDYWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGCGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 348 - protein, 708 aa, synthetic construct
  REGION 1..708: note = Description of Artificial Sequence: Synthetic polypeptide
EVQLLESGGGLVQPGGSLRLSCAASGFTFSSYAMSWVRQAPGKCLEWVSAISGSGGSTYYADSVKGRFTISRDNSKNTLYLQMNSLRAEDTAVYYCARREWWGESWLFDYWGQGTLVTVSSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSDIQMTQSPSSLSASVGDRVTITCRASQSISSYLNWYQQKPGKAPKLLIYAASSLQSGVPSRFSGSGSGTDFTLTISSLQPEDFATYYCQQSYSTPLTFGCGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 349 - protein, 486 aa, synthetic construct
  REGION 1..486: note = Description of Artificial Sequence: Synthetic polypeptide
MALPVTALLLPLALLLHAARPEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 350 - protein, 465 aa, synthetic construct; identical to SEQ ID NO: 225
  REGION 1..465: note = Description of Artificial Sequence: Synthetic polypeptide

SEQ ID NO: 351 - protein, 22 aa, synthetic construct
  REGION 1..22: note = Description of Artificial Sequence: Synthetic peptide
MLLLVTSLLLCELPHPAFLLIP

SEQ ID NO: 352 - DNA, 66 bp, synthetic construct
  misc_feature 1..66: note = Description of Artificial Sequence: Synthetic oligonucleotide
  source 1..66: mol_type = other DNA
atgcttctcctggtgacaagccttctgctctgtgagttaccacacccagcattcctcctgatccca

SEQ ID NO: 353 - DNA, 66 bp, synthetic construct
  misc_feature 1..66: note = Description of Artificial Sequence: Synthetic oligonucleotide
  source 1..66: mol_type = other DNA
atgctgctgctggtgaccagcctgctgctgtgcgagctgccccaccccgcctttctgctgatcccc

SEQ ID NO: 354 - DNA, 1461 bp, synthetic construct
  misc_feature 1..1461: note = Description of Artificial Sequence: Synthetic polynucleotide
  source 1..1461: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 355 - DNA, 1458 bp, synthetic construct
  misc_feature 1..1458: note = Description of Artificial Sequence: Synthetic polynucleotide
  source 1..1458: mol_type = other DNA
atggccctccctgtcaccgccctgctgcttccgctggctcttctgctccacgccgctcggcccgaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

SEQ ID NO: 356 - DNA, 1398 bp, synthetic construct
  misc_feature 1..1398: note = Description of Artificial Sequence: Synthetic polynucleotide
  source 1..1398: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcggtaa

SEQ ID NO: 357 - DNA, 360 bp, synthetic construct
  misc_feature 1..360: note = Description of Artificial Sequence: Synthetic polynucleotide
  source 1..360: mol_type = other DNA
caggtccagctgcaggaatcaggaccagggctggtgaaacctagcgaaactctgagtctgacttgtaccgtctccggggtgtctctgccagactacggcgtgagctggatcagacagccccctggcaagtgcctggagtggatcggcgtgatctggggctccgagaccacatactatcagagctccctgaagtctcgggtgaccatctccaaggacaactctaagaatcaggtgagcctgaagctgtctagcgtgaccgccgccgatacagccgtgtactattgtgccaagcactactattacggcggctcctatgccatggattactggggccagggcaccctggtgacagtgtcctct

SEQ ID NO: 358 - DNA, 360 bp, synthetic construct
  misc_feature 1..360: note = Description of Artificial Sequence: Synthetic polynucleotide
  source 1..360: mol_type = other DNA
caggtccagctgcaggaatccggcccaggactggtgaagcctagcgagaccctgtccctgacctgcacagtgagcggcgtgtccctgcccgattacggcgtgagctggatcagacagccccctggcaagtgtctggagtggatcggcgtgatctggggctctgagaccacatactatcagtcctctctgaagagcagggtgaccatctctaaggacaacagcaagaatcaggtgtccctgaagctgagctccgtgaccgcagcagatacagccgtgtactattgcgccaagcactactattacggcggctcctatgctatggattattgggggcagggcactctggtcactgtctcatca

SEQ ID NO: 359 - DNA, 360 bp, synthetic construct
  misc_feature 1..360: note = Description of Artificial Sequence: Synthetic polynucleotide
  source 1..360: mol_type = other DNA
caggtgcagctgcaggaatctggacccggactggtgaaacctagtgaaactctgtctctgacttgtaccgtctcaggggtctcactgccagactacggcgtgtcctggatcagacagccccctggcaagtgcctggagtggatcggcgtgatctggggctctgagaccacatactatcagagctccctgaagagccgggtgaccatctccaaggacaactctaagaatcaggtgtccctgaagctgtctagcgtgaccgccgccgatacagccgtgtactattgtgccaagcactactattacggcggcagctatgccatggattactggggccagggcaccctggtgacagtgtcctct

SEQ ID NO: 360 - DNA, 360 bp, synthetic construct
  misc_feature 1..360: note = Description of Artificial Sequence: Synthetic polynucleotide
  source 1..360: mol_type = other DNA
caggtccagctgcaggaaagcggcccaggactggtgaagcctagcgagaccctgtccctgacctgcacagtgagcggcgtgtccctgcctgattacggcgtgtcctggatcagacagccccctggcaagtgtctggagtggatcggcgtgatctggggctccgagaccacatactatcagtcctctctgaagtctagggtgacaatctctaaggacaacagcaagaatcaggtgagcctgaagctgagctccgtgaccgcagcagatacagccgtgtactattgtgccaagcactactattacggcggctcttatgctatggattattgggggcagggcactctggtcactgtctcaagc

SEQ ID NO: 361 - DNA, 360 bp, synthetic construct
  misc_feature 1..360: note = Description of Artificial Sequence: Synthetic polynucleotide
  source 1..360: mol_type = other DNA
caggtgcagctgcaggagagcggcccaggactggtgaagccttccgagacactgtctctgacctgtacagtgagcggcgtgtccctgcccgactacggcgtgtcctggatcagacagccacctggcaagggactggagtggatcggcgtgatctggggcagcgagaccacatactatcagagctccctgaagtccagggtgaccatcagcaaggacaactccaagaatcaggtgagcctgaagctgtctagcgtgaccgccgccgatacagccgtgtactattgcgccaagcactactattacggcggctcctatgccatggattactggggccagggcaccctggtcacagtgtcctct

SEQ ID NO: 362 - DNA, 360 bp, synthetic construct
  misc_feature 1..360: note = Description of Artificial Sequence: Synthetic polynucleotide
  source 1..360: mol_type = other DNA
caggtgcagctgcaggagtctggcccaggactggtgaagccttctgagaccctgagcctgacctgcacagtgtccggcgtgtctctgcccgattacggcgtgtcctggatcagacagccacctggcaagggactggagtggatcggcgtgatctggggctctgagaccacatactatcagtctagcctgaagagccgggtgacaatctccaaggacaactctaagaatcaggtgtccctgaagctgtcctctgtgaccgccgccgatacagccgtgtactattgtgccaagcactactattacggcggcagctatgccatggactactggggccagggcaccctggtgacagtgagctcc

SEQ ID NO: 363 - DNA, 360 bp, synthetic construct
  misc_feature 1..360: note = Description of Artificial Sequence: Synthetic polynucleotide
  source 1..360: mol_type = other DNA
caggtgcagctgcaggagtctggcccaggactggtgaagccttctgagaccctgagcctgacctgcacagtgagcggcgtgtccctgcccgattacggcgtgtcctggatcagacagccacctggcaagggactggagtggatcggcgtgatctggggcagcgagaccacatactatcagtcctctctgaagtccagggtgacaatctccaaggacaactctaagaatcaggtgagcctgaagctgagctccgtgaccgcagcagatacagccgtgtactattgcgccaagcactactattacggcggctcctatgccatggactactggggccagggcaccctggtcacagtgtctagc

SEQ ID NO: 364 - DNA, 360 bp, synthetic construct
  misc_feature 1..360: note = Description of Artificial Sequence: Synthetic polynucleotide
  source 1..360: mol_type = other DNA
caggtgcagctgcaggagtccggcccaggactggtgaagccttccgagacactgtctctgacctgtacagtgtccggcgtgtctctgcccgactacggcgtgagctggatcagacagccacctggcaagggactggagtggatcggcgtgatctggggctctgagaccacatactatcagtcctctctgaagagccgggtgaccatcagcaaggacaactccaagaatcaggtgtccctgaagctgagctccgtgaccgcagcagatacagccgtgtactattgcgccaagcactactattacggcggcagctatgccatggattactggggccagggcaccctggtgacagtgtctagc

SEQ ID NO: 365 - DNA, 321 bp, synthetic construct
  misc_feature 1..321: note = Description of Artificial Sequence: Synthetic polynucleotide
  source 1..321: mol_type = other DNA
gagatcgtgatgacccagagcccagccacactgagcctgtccccaggagagagggccacactgtcttgtagagccagccaggatatctccaagtatctgaactggtaccagcagaagcctggacaggcaccaaggctgctgatctaccacacctctagactgcacagcggcatccctgccaggttttctggcagcggctccggcacagactataccctgacaatctctagcctgcagccagaggatttcgccgtgtacttttgtcagcagggcaatactctgccatacacctttggatgcggaactaaactggaaatcaag

SEQ ID NO: 366 - DNA, 321 bp, synthetic construct
  misc_feature 1..321: note = Description of Artificial Sequence: Synthetic polynucleotide
  source 1..321: mol_type = other DNA
gaaattgtgatgacccagtcccccgctactctgtctctgtcccccggagaacgggctactctgtcttgtcgcgcttcccaggatattagcaagtacctgaactggtatcagcagaagccaggacaggcaccaaggctgctgatctaccacacctctcgcctgcacagcggcatccctgcacggttctctggcagcggctccggcacagactacaccctgacaatcagctccctgcagcctgaggatttcgccgtgtacttttgccagcagggcaataccctgccatatacatttggctgtggcaccaagctggagatcaag

SEQ ID NO: 367 - DNA, 321 bp, synthetic construct
  misc_feature 1..321: note = Description of Artificial Sequence: Synthetic polynucleotide
  source 1..321: mol_type = other DNA
gagatcgtgatgacccagtccccagccacactgagcctgtccccaggagagagggccaccctgtcttgtagagccagccaggatatctccaagtatctgaactggtaccagcagaagcctggacaggcaccaaggctgctgatctaccacacctctagactgcacagcggcatccctgccaggttttctggcagcggctccggcacagactataccctgacaatctctagcctgcagccagaggatttcgccgtgtacttttgtcagcagggaaatactctgccatacacctttggatgcggaactaaactggaaatcaag

SEQ ID NO: 368 - DNA, 321 bp, synthetic construct
  misc_feature 1..321: note = Description of Artificial Sequence: Synthetic polynucleotide
  source 1..321: mol_type = other DNA
gagattgtgatgacccagtcccccgccaccctgagtctgagccccggagaacgagctaccctgagttgccgagcttcccaggacatttccaagtacctgaactggtatcagcagaagccaggacaggcaccaaggctgctgatctaccacacctctcgcctgcacagcggcatcccagcacggttctctggcagcggctccggcacagactacaccctgacaatcagctccctgcagcctgaggatttcgccgtgtacttttgccagcagggcaataccctgccatatacatttggctgtggcaccaagctggagatcaag

SEQ ID NO: 369 - DNA, 321 bp, synthetic construct
  misc_feature 1..321: note = Description of Artificial Sequence: Synthetic polynucleotide
  source 1..321: mol_type = other DNA
gagatcgtgatgacccagtctccagccacactgtctctgagcccaggagagagggccaccctgtcttgccgcgccagccaggatatctccaagtatctgaactggtaccagcagaagccaggacaggcaccaaggctgctgatctaccacacctctcgcctgcacagcggcatcccagcacggttctccggctctggcagcggcacagactacaccctgacaatctcctctctgcagcccgaggatttcgccgtgtatttttgccagcagggcaataccctgccttacacatttggccagggcaccaagctggagatcaag

SEQ ID NO: 370 - DNA, 321 bp, synthetic construct
  misc_feature 1..321: note = Description of Artificial Sequence: Synthetic polynucleotide
  source 1..321: mol_type = other DNA
gagatcgtgatgacccagagcccagccacactgagcctgtccccaggagagagggccaccctgagctgcagagcctcccaggatatctctaagtatctgaactggtaccagcagaagcctggacaggcaccaaggctgctgatctaccacaccagcagactgcactccggcatccctgcaaggttctctggcagcggctccggcacagactacaccctgacaatctctagcctgcagcctgaggatttcgccgtgtatttttgtcagcagggcaataccctgccatacacatttggccagggcaccaagctggagatcaag

SEQ ID NO: 371 - DNA, 321 bp, synthetic construct
  misc_feature 1..321: note = Description of Artificial Sequence: Synthetic polynucleotide
  source 1..321: mol_type = other DNA
gagatcgtgatgacccagagcccagccacactgtctctgagcccaggagagagggccaccctgagctgtcgcgcctcccaggatatctctaagtatctgaactggtaccagcagaagccaggacaggcaccaaggctgctgatctaccacaccagccgcctgcactccggcatcccagcacggttctccggctctggcagcggcacagactacaccctgacaatctcctctctgcagcccgaggatttcgccgtgtatttttgccagcagggcaataccctgccttacacatttggccagggcaccaagctggagatcaag

SEQ ID NO: 372 - DNA, 321 bp, synthetic construct
  misc_feature 1..321: note = Description of Artificial Sequence: Synthetic polynucleotide
  source 1..321: mol_type = other DNA
gagatcgtgatgacccagtctccagccacactgagcctgtccccaggagagagggccaccctgtcttgcagagccagccaggatatctccaagtatctgaactggtaccagcagaagcctggacaggcaccaaggctgctgatctaccacacctctagactgcacagcggcatcccagcaaggttctctggcagcggctccggcacagactacaccctgacaatctctagcctgcagcctgaggatttcgccgtgtatttttgccagcagggcaataccctgccatacacatttggccagggcaccaagctggagatcaag

SEQ ID NO: 373 - protein, 242 aa, synthetic construct
  REGION 1..242: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 374 - protein, 242 aa, synthetic construct
  REGION 1..242: note = Description of Artificial Sequence: Synthetic polypeptide
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 375 - protein, 242 aa, synthetic construct
  REGION 1..242: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 376 - protein, 247 aa, synthetic construct
  REGION 1..247: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSS

SEQ ID NO: 377 - protein, 247 aa, synthetic construct
  REGION 1..247: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSS

SEQ ID NO: 378 - protein, 247 aa, synthetic construct
  REGION 1..247: note = Description of Artificial Sequence: Synthetic polypeptide
QVQLQESGPGLVKPSETLSLTCTVSGVSLPDYGVSWIRQPPGKGLEWIGVIWGSETTYYSSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 379 - protein, 247 aa, synthetic construct
  REGION 1..247: note = Description of Artificial Sequence: Synthetic polypeptide
QVQLQESGPGLVKPSETLSLTCTVSGVSLPDYGVSWIRQPPGKGLEWIGVIWGSETTYYQSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 380 - protein, 247 aa, synthetic construct
  REGION 1..247: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 381 - protein, 247 aa, synthetic construct
  REGION 1..247: note = Description of Artificial Sequence: Synthetic polypeptide
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 382 - protein, 242 aa, synthetic construct
  REGION 1..242: note = Description of Artificial Sequence: Synthetic polypeptide
EIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIKGGGGSGGGGSGGGGSQVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSS

SEQ ID NO: 383 - protein, 242 aa, synthetic construct
  REGION 1..242: note = Description of Artificial Sequence: Synthetic polypeptide
QVQLQESGPGLVKPSETLSLTCTVSGVSLPDYGVSWIRQPPGKGLEWIGVIWGSETTYYNSSLKSRVTISKDNSKNQVSLKLSSVTAADTAVYYCAKHYYYGGSYAMDYWGQGTLVTVSSGGGGSGGGGSGGGGSEIVMTQSPATLSLSPGERATLSCRASQDISKYLNWYQQKPGQAPRLLIYHTSRLHSGIPARFSGSGSGTDYTLTISSLQPEDFAVYFCQQGNTLPYTFGQGTKLEIK

SEQ ID NO: 384 - DNA, 1467 bp, synthetic construct
  misc_feature 1..1467: note = Description of Artificial Sequence: Synthetic polynucleotide
  source 1..1467: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 385 - protein, 489 aa, synthetic construct
  REGION 1..489: note = Description of Artificial Sequence: Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 386 - DNA, 801 bp, synthetic construct
  misc_feature 1..801: note = Description of Artificial Sequence: Synthetic polynucleotide
  source 1..801: mol_type = other DNA
atgcttctcctggtgacaagccttctgctctgtgagttaccacacccagcattcctcctgatcccagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 387 - protein, 266 aa, synthetic construct
  REGION 1..266: note = Description of Artificial Sequence: Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 388 - DNA, 1401 bp, synthetic construct
  misc_feature 1..1401: note = Description of Artificial Sequence: Synthetic polynucleotide
  source 1..1401: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcagcggccgcaattgaagttatgtatcctcctccttacctagacaatgagaagagcaatggaaccattatccatgtgaaagggaaacacctttgtccaagtcccctatttcccggaccttctaagcccttttgggtgctggtggtggttgggggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 389 - protein, 467 aa, synthetic construct
  REGION 1..467: note = Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 390 - DNA, 735 bp, synthetic construct
  misc_feature 1..735: note = Description of Artificial Sequence: Synthetic polynucleotide
  source 1..735: mol_type = other DNA
gacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggactaagttggaaataacaggctccacctctggatccggcaagcccggatctggcgagggatccaccaagggcgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctca

SEQ ID NO: 391 - protein, 245 aa, synthetic construct
  REGION 1..245: note = Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 392 - DNA, 1383 bp, synthetic construct
  misc_feature 1..1383: note = Description of Artificial Sequence: Synthetic polynucleotide
  source 1..1383: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 393 - protein, 461 aa, synthetic construct
  REGION 1..461: note = Description of Artificial Sequence: Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 394 - DNA, 801 bp, synthetic construct
  misc_feature 1..801: note = Description of Artificial Sequence: Synthetic polynucleotide
  source 1..801: mol_type = other DNA
atgctgctgctggtgaccagcctgctgctgtgcgagctgccccaccccgcctttctgctgatccccgacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 395 - protein, 267 aa, synthetic construct
  REGION 1..267: note = Description of Artificial Sequence: Synthetic polypeptide
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 396 - DNA, 1317 bp, synthetic construct
  misc_feature 1..1317: note = Description of Artificial Sequence: Synthetic polynucleotide
  source 1..1317: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagcgaatctaagtacggaccgccctgccccccttgccctatgttctgggtgctggtggtggtcggaggcgtgctggcctgctacagcctgctggtcaccgtggccttcatcatcttttgggtgaaacggggcagaaagaaactcctgtatatattcaaacaaccatttatgagaccagtacaaactactcaagaggaagatggctgtagctgccgatttccagaagaagaagaaggaggatgtgaactgcgggtgaagttcagcagaagcgccgacgcccctgcctaccagcagggccagaatcagctgtacaacgagctgaacctgggcagaagggaagagtacgacgtcctggataagcggagaggccgggaccctgagatgggcggcaagcctcggcggaagaacccccaggaaggcctgtataacgaactgcagaaagacaagatggccgaggcctacagcgagatcggcatgaagggcgagcggaggcggggcaagggccacgacggcctgtatcagggcctgtccaccgccaccaaggatacctacgacgccctgcacatgcaggccctgcccccaagg

SEQ ID NO: 397 - protein, 439 aa, synthetic construct
  REGION 1..439: note = Description of Artificial Sequence: Synthetic polypeptide
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSESKYGPPCPPCPMFWVLVVVGGVLACYSLLVTVAFIIFWVKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 398 - DNA, 735 bp, synthetic construct
  misc_feature 1..735: note = Description of Artificial Sequence: Synthetic polynucleotide
  source 1..735: mol_type = other DNA
gacatccagatgacccagaccacctccagcctgagcgccagcctgggcgaccgggtgaccatcagctgccgggccagccaggacatcagcaagtacctgaactggtatcagcagaagcccgacggcaccgtcaagctgctgatctaccacaccagccggctgcacagcggcgtgcccagccggtttagcggcagcggctccggcaccgactacagcctgaccatctccaacctggaacaggaagatatcgccacctacttttgccagcagggcaacacactgccctacacctttggcggcggaacaaagctggaaatcaccggcagcacctccggcagcggcaagcctggcagcggcgagggcagcaccaagggcgaggtgaagctgcaggaaagcggccctggcctggtggcccccagccagagcctgagcgtgacctgcaccgtgagcggcgtgagcctgcccgactacggcgtgagctggatccggcagccccccaggaagggcctggaatggctgggcgtgatctggggcagcgagaccacctactacaacagcgccctgaagagccggctgaccatcatcaaggacaacagcaagagccaggtgttcctgaagatgaacagcctgcagaccgacgacaccgccatctactactgcgccaagcactactactacggcggcagctacgccatggactactggggccagggcaccagcgtgaccgtgagcagc

SEQ ID NO: 399 - protein, 245 aa, synthetic construct; identical to SEQ ID NO: 391
  REGION 1..245: note = Description of Artificial Sequence: Synthetic polypeptide

SEQ ID NO: 400 - DNA, 369 bp, synthetic construct
  misc_feature 1..369: note = Description of Artificial Sequence: Synthetic polynucleotide
  source 1..369: mol_type = other DNA
caggtgcagctgcaggagtccggcggcggcgtggtgcagccaggccggtccctgagactgtcttgtgccgccagcggcttcaccttttcctcttatggcatgcactgggtgagacaggcacctggcaagggactggagtgggtggccgtgatctcctacgacggctctaacaagtattacgccgatagcgtgaagggcaggttcaccatcagccgcgacaactccaagaatacactgtatctgcagatgaatagcctgcgggccgaggataccgccgtgtattactgcggaggctccggctacgcactgcacgacgattattacggactggacgtgtggggacagggcaccctggtcacagtgagctcc

SEQ ID NO: 401 - DNA, 369 bp, synthetic construct
  misc_feature 1..369: note = Description of Artificial Sequence: Synthetic polynucleotide
  source 1..369: mol_type = other DNA
caggtgcagctgcaggagtctggcggaggagtggtgcagccaggccggtccctgagactgtcttgcgccgccagcggcttcacattttctagctacggaatgcactgggtgcgccaggcacctggcaagggactggagtgggtggccgtgatctcctatgacggctctaacaagtactatgccgattccgtgaagggcaggttcaccatcagccgcgacaactccaagaatacactgtacctgcagatgaattccctgcgggccgaggataccgccgtgtactattgtggcggctctggctatgccctgcacgacgattactatggactggacgtgtggggacagggcaccctggtgacagtgtcctct

SEQ ID NO: 402 - DNA, 369 bp, synthetic construct
  misc_feature 1..369: note = Description of Artificial Sequence: Synthetic polynucleotide
  source 1..369: mol_type = other DNA
caggtgcagctgcaggagtctggcggaggagtggtgcagccaggccggagcctgagactgtcctgcgccgcctctggcttcacctttagctcctatggcatgcactgggtgagacaggcacctggcaagggactggagtgggtggccgtgatcagctacgacggctccaacaagtattacgccgatagcgtgaagggcaggttcaccatctctcgcgacaacagcaagaatacactgtatctgcagatgaattccctgcgggccgaggatacagccgtgtattactgcggaggcagcggctacgcactgcacgacgattattacggactggacgtgtggggacagggcaccctggtcacagtgtctagc

SEQ ID NO: 403 - DNA, 369 bp, synthetic construct
  misc_feature 1..369: note = Description of Artificial Sequence: Synthetic polynucleotide
  source 1..369: mol_type = other DNA
caggtgcagctgcaggagagcggcggcggcgtggtgcagcccggccggtctctgagactgagctgtgccgcctccggcttcacctttagctcctacggaatgcactgggtgcgccaggcacctggcaagggactggagtgggtggccgtgatctcttatgacggcagcaacaagtactatgccgatagcgtgaagggcaggttcaccatctcccgcgacaactctaagaatacactgtacctgcagatgaatagcctgcgggccgaggataccgccgtgtactattgcggaggctccggctatgcactgcacgacgattactatggactggacgtgtggggacagggcaccctggtgacagtgtctagc

SEQ ID NO: 404 - DNA, 369 bp, synthetic construct
  misc_feature 1..369: note = Description of Artificial Sequence: Synthetic polynucleotide
  source 1..369: mol_type = other DNA
caggtccagctgcaggagagtggggggggggtcgtccagcccggaagaagcctgagactgtcatgtgccgcatctgggtttacttttagctcctatggaatgcactgggtgcgccaggcacctggcaagtgcctggagtgggtggccgtgatctcctacgacggctctaacaagtactatgccgatagcgtgaagggccggttcaccatcagcagagacaactccaagaatacactgtatctgcagatgaattctctgcgggccgaggataccgccgtgtactattgtggaggctccggctacgcactgcacgacgattactatggactggacgtgtggggacagggcaccctggtgacagtgtctagc

SEQ ID NO: 405 - DNA, 369 bp, synthetic construct
  misc_feature 1..369: note = Description of Artificial Sequence: Synthetic polynucleotide
  source 1..369: mol_type = other DNA
caggtccagctgcaggaatccggcggaggagtggtgcagccaggccggtctctgagactgagctgcgccgcctccggcttcacattttcctcttatggcatgcactgggtgagacaggcccctggcaagtgtctggagtgggtggccgtgatctcttacgacggcagcaacaagtattacgccgatagcgtgaagggcaggttcaccatctcccgcgacaactctaagaatacactgtatctgcagatgaattccctgcgggccgaggataccgccgtgtattactgcggcggctctggctacgccctgcacgacgactactatggactggatgtctgggggcagggcacactggtcactgtctcttca

SEQ ID NO: 406 - DNA, 369 bp, synthetic construct
  misc_feature 1..369: note = Description of Artificial Sequence: Synthetic polynucleotide
  source 1..369: mol_type = other DNA
caggtccagctgcaggaatcaggggggggggtcgtccagcccggaagaagtctgagactgtcatgtgccgcatcagggtttacctttagctcctatggaatgcactgggtgcgccaggcacctggcaagtgcctggagtgggtggccgtgatctcctacgacggctctaacaagtactatgccgatagcgtgaagggccggttcaccatcagcagagacaactccaagaatacactgtatctgcagatgaattctctgcgggccgaggataccgccgtgtactattgtggaggctccggctacgcactgcacgacgattactatggactggacgtgtggggacagggcaccctggtgacagtgtctagc

SEQ ID NO: 407 - DNA, 369 bp, synthetic construct
  misc_feature 1..369: note = Description of Artificial Sequence: Synthetic polynucleotide
  source 1..369: mol_type = other DNA
caggtccagctgcaggaatccggcggaggagtggtgcagccaggccggtctctgagactgagctgcgccgcctccggcttcaccttttcctcttatggcatgcactgggtgagacaggcccctggcaagtgtctggagtgggtggccgtgatctcttacgacggcagcaacaagtattacgccgatagcgtgaagggcaggttcaccatctcccgcgacaactctaagaatacactgtatctgcagatgaattccctgcgggccgaggatacagccgtgtattactgtggcggctctggctacgccctgcatgatgattattatggactggatgtctgggggcagggcacactggtcactgtctcttcc

SEQ ID NO: 408 - DNA, 333 bp, synthetic construct
  misc_feature 1..333: note = Description of Artificial Sequence: Synthetic polynucleotide
  source 1..333: mol_type = other DNA
cagtctgccctgacccagccagcaagcgtgtccggctctcctggccagagcatcacaatctcctgcaccggcacaagctccgacgtgggaggctataactacgtgagctggtatcagcagcacccaggcaaggcccccaagctgatgatctacgacgtgagcaacaggccttctggcgtgagcaatcgcttcagcggctccaagtctggcaataccgcctctctgacaatcagcggcctgcaggcagaggacgaggcagattattactgctctagctatacctcctctagcacactgtacgtgtttggcagcggcaccaaggtgacagtgctg

SEQ ID NO: 409 - DNA, 333 bp, synthetic construct
  misc_feature 1..333: note = Description of Artificial Sequence: Synthetic polynucleotide
  source 1..333: mol_type = other DNA
cagagcgccctgacccagccagcatccgtgtctggcagcccaggccagtctatcacaatcagctgcaccggcacaagctccgacgtgggaggctacaactatgtgagctggtaccagcagcaccctggcaaggccccaaagctgatgatctatgacgtgagcaaccggccatccggcgtgtctaatagattctccggctctaagagcggcaataccgcctccctgacaatctctggcctgcaggcagaggacgaggcagattactattgttctagctacacctcctctagcacactgtacgtgttcggcagcggcaccaaggtgacagtgctg

SEQ ID NO: 410 - DNA, 333 bp, synthetic construct
  misc_feature 1..333: note = Description of Artificial Sequence: Synthetic polynucleotide
  source 1..333: mol_type = other DNA
cagtctgccctgacccagccagcaagcgtgtccggctctcctggccagtccatcacaatctcttgtaccggcacatcctctgacgtgggcggctataactacgtgtcctggtatcagcagcacccaggcaaggcccccaagctgatgatctacgatgtgagcaacaggccttctggcgtgagcaatcgcttcagcggctccaagtctggcaataccgccagcctgacaatctccggcctgcaggcagaggacgaggcagattattactgcagctcctatacctctagctccacactgtacgtgtttggcagcggcaccaaggtgacagtgctg

SEQ ID NO: 411 - DNA, 333 bp, synthetic construct
  misc_feature 1..333: note = Description of Artificial Sequence: Synthetic polynucleotide
  source 1..333: mol_type = other DNA
cagagcgccctgacccagccagcatccgtgtctggcagcccaggccagtccatcacaatctcttgcaccggcacatctagcgacgtgggcggctacaactacgtgagctggtaccagcagcaccctggcaaggccccaaagctgatgatctatgatgtgagcaaccggccctccggcgtgtctaatagattctccggctctaagagcggcaataccgccagcctgacaatctccggcctgcaggcagaggacgaggcagattactattgctcctcttacaccagctcctctacactgtacgtgttcggctccggcaccaaggtgacagtgctg

SEQ ID NO: 412 - DNA, 333 bp, synthetic construct
  misc_feature 1..333: note = Description of Artificial Sequence: Synthetic polynucleotide
  source 1..333: mol_type = other DNA
cagtctgccctgacccagcctgcaagcgtgtccggctctccaggccagtctatcacaatcagctgtaccggcacaagctccgacgtgggcggctataactacgtgagctggtatcagcagcaccctggcaaggccccaaagctgatgatctacgacgtgagcaaccggccctctggcgtgagcaatcggttcagcggcagcaagtctggcaataccgcctccctgacaatctctggcctgcaggcagaggacgaggcagattattactgtagcagttatacttcaagctcaaccctgtacgtgtttggatgcggcactaaggtcaccgtcctg

SEQ ID NO: 413 - DNA, 333 bp, synthetic construct
  misc_feature 1..333: note = Description of Artificial Sequence: Synthetic polynucleotide
  source 1..333: mol_type = other DNA
cagtctgctctgacccagcccgcttccgtctcagggtctccaggacagtcaattaccattagttgcacaggcacctcatccgatgtgggcggctataactacgtgtcctggtatcagcagcacccaggcaaggcccccaagctgatgatctacgacgtgagcaacaggccatctggcgtgagcaatcgcttcagcggctccaagtctggcaataccgccagcctgacaatctccggcctgcaggcagaggacgaggcagattactattgcagctcctatacctctagctccacactgtacgtgtttggctgtggcaccaaggtgacagtgctg

SEQ ID NO: 414 - DNA, 333 bp, synthetic construct
  misc_feature 1..333: note = Description of Artificial Sequence: Synthetic polynucleotide
  source 1..333: mol_type = other DNA
cagtctgccctgacccagcctgcaagcgtgtccggctctccaggccagtctatcacaatcagctgtaccggcacaagctccgacgtgggcggctataactacgtgagctggtatcagcagcaccctggcaaggccccaaagctgatgatctacgacgtgagcaaccggccctctggcgtgagcaatcggttcagcggcagcaagtctggcaataccgcctccctgacaatctctggcctgcaggcagaggacgaggcagattattactgtagctcctacacttcttcaagcacactgtatgtctttggatgcggaactaaggtcactgtcctg

SEQ ID NO: 415 - DNA, 333 bp, synthetic construct
  misc_feature 1..333: note = Description of Artificial Sequence: Synthetic polynucleotide
  source 1..333: mol_type = other DNA
cagtctgctctgacccagcccgcttccgtctcaggatctccaggacagtctattacaattagttgcacaggaacctcttccgatgtgggcggctataactacgtgtcctggtatcagcagcacccaggcaaggcccccaagctgatgatctacgacgtgagcaacaggccttctggcgtgagcaatcgcttcagcggctccaagtctggcaataccgccagcctgacaatctccggcctgcaggcagaggacgaggcagattactattgcagctcctatacctctagctccacactgtacgtgtttggctgtggcaccaaggtgacagtgctg

SEQ ID NO: 416 - DNA, 1485 bp, synthetic construct
  misc_feature 1..1485: note = Description of Artificial Sequence: Synthetic polynucleotide
  source 1..1485: mol_type = other DNA
atggccctccctgtcaccgctctgttgctgccgcttgctctgctgctccacgcagcgcgaccgcaggtacaattgcaggagtctggaggcggtgtggtgcaacccggtcgcagcttgcgcctgagttgtgctgcgtctggatttacattttcatcttacggaatgcattgggtacgccaggcaccggggaaaggccttgaatgggtggctgtaatttcatacgatggttccaacaaatactatgctgactcagtcaagggtcgatttacaattagtcgggacaactccaagaacaccctttatcttcaaatgaattcccttagagcagaggatacggcggtctattactgtggtggcagtggttatgcacttcatgatgattactatggcttggatgtctgggggcaagggacgcttgtaactgtatcctctggtggtggtggtagtggtgggggaggctccggcggtggcggctctcaatctgctctgactcaaccagcaagcgtatcagggtcaccgggacagagtattaccataagttgcacggggacctctagcgatgtaggggggtataattatgtatcttggtatcaacaacaccccgggaaagcccctaaattgatgatctacgacgtgagcaatcgacctagtggcgtatcaaatcgcttctctggtagcaagagtgggaatacggcgtcccttactattagcggattgcaagcagaagatgaggccgattactactgcagctcctatactagctcttctacattgtacgtctttgggagcggaacaaaagtaacagtactcacaacaacacctgccccgagaccgcctacaccagccccgactattgccagccagcctctgagcctcaggcctgaggcctgtaggcccgcagcgggcggcgcagttcatacacggggcttggatttcgcttgtgatatttatatttgggctcctttggcggggacatgtggcgtgctgcttctgtcacttgttattacactgtactgtaaacgcgggcgaaaaaaattgctgtatatttttaagcagccatttatgaggcccgttcagacgacgcaggaggaggacggttgctcttgcaggttcccagaagaggaagaagggggctgtgaattgcgggttaaattttcaagatccgcagacgctccagcataccaacagggacaaaaccaactctataacgagctgaatcttggaagaagggaggaatatgatgtgctggataaacggcgcggtagagatccggagatgggcggaaaaccaaggcgaaaaaaccctcaggagggactctacaacgaactgcagaaagacaaaatggcggaggcttattccgaaataggcatgaagggcgagcggaggcgagggaaagggcacgacggactgtatcaaggcctctcaaccgcgactaaggatacgtacgacgccctgcacatgcaggccctgcctccgagatgataa

SEQ ID NO: 417 - DNA, 1395 bp, synthetic construct
  misc_feature 1..1395: note = Description of Artificial Sequence: Synthetic polynucleotide
  source 1..1395: mol_type = other DNA
gaaattgtgatgacccagtcacccgccactcttagcctttcacccggtgagcgcgcaaccctgtcttgcagagcctcccaagacatctcaaaataccttaattggtatcaacagaagcccggacaggctcctcgccttctgatctaccacaccagccggctccattctggaatccctgccaggttcagcggtagcggatctgggaccgactacaccctcactatcagctcactgcagccagaggacttcgctgtctatttctgtcagcaagggaacaccctgccctacacctttggacagggcaccaagctcgagattaaaggtggaggtggcagcggaggaggtgggtccggcggtggaggaagccaggtccaactccaagaaagcggaccgggtcttgtgaagccatcagaaactctttcactgacttgtactgtgagcggagtgtctctccccgattacggggtgtcttggatcagacagccaccggggaagggtctggaatggattggagtgatttggggctctgagactacttactaccaatcatccctcaagtcacgcgtcaccatctcaaaggacaactctaagaatcaggtgtcactgaaactgtcatctgtgaccgcagccgacaccgccgtgtactattgcgctaagcattactattatggcgggagctacgcaatggattactggggacagggtactctggtcaccgtgtccagcaccactaccccagcaccgaggccacccaccccggctcctaccatcgcctcccagcctctgtccctgcgtccggaggcatgtagacccgcagctggtggggccgtgcatacccggggtcttgacttcgcctgcgatatctacatttgggcccctctggctggtacttgcggggtcctgctgctttcactcgtgatcactctttactgtaagcgcggtcggaagaagctgctgtacatctttaagcaacccttcatgaggcctgtgcagactactcaagaggaggacggctgttcatgccggttcccagaggaggaggaaggcggctgcgaactgcgcgtgaaattcagccgcagcgcagatgctccagcctaccagcaggggcagaaccagctctacaacgaactcaatcttggtcggagagaggagtacgacgtgctggacaagcggagaggacgggacccagaaatgggcgggaagccgcgcagaaagaatccccaagagggcctgtacaacgagctccaaaaggataagatggcagaagcctatagcgagattggtatgaaaggggaacgcagaagaggcaaaggccacgacggactgtaccagggactcagcaccgccaccaaggacacctatgacgctcttcacatgcaggccctgccgcctcgg

(19) United States
(12) Patent Application Publication
(10) Pub. No.: US 2026/0034195 A1
(43) Pub. Date: Feb. 5, 2026

(54) CHIMERIC ANTIGEN RECEPTORS AND USES THEREOF

(71) Applicant: Novartis AG, Basel (CH)

(72) Inventors: Aida Abujoub, Winchester, MA (US); John Blankenship, Acton, MA (US); Attilio Bondanza, Basel (CH); Jennifer Brogdon, Sudbury, MA (US); Dexiu Bu, Melrose, MA (US); Serena De Vita, Brookline, MA (US); Glenn Dranoff, Sudbury, MA (US); Boris Engels, Arlington, MA (US); Tony Fleming, Stow, MA (US); Brian Walter Granda, Salisbury, MA (US); Michael R. Greene, Groton, MA (US); Carla Patricia Guimaraes, Boston, MA (US); Anniesha Hack, East Hanover, NJ (US); Brian Holmberg, Somerville, MA (US); Connie Hong, Somerville, MA (US); Lu Huang, West Roxbury, MA (US); Olja Kodrasi, Quincy, MA (US); Joni Waiee Lam, Somerville, MA (US); Hyungwook Lim, Cambridge, MA (US); Elizabeth Dorothy Pratico, Watertown, MA (US); Andrew Price, Cambridge, MA (US); Akash Sohoni, Cambridge, MA (US); Andrew Marc Stein, Cambridge, MA (US); Louis Treanor, Cambridge, MA (US); Chonghui Zhang, Cambridge, MA (US); Xu Zhu, Cambridge, MA (US)

(21) Appl. No.: 19/262,665

(22) Filed: Jul. 8, 2025

Related U.S. Application Data
(63) Continuation of application No. 17/104,983, filed on Nov. 25, 2020, now Pat. No. 12,383,601.
(60) Provisional application No. 62/940,509, filed on Nov. 26, 2019.

(51) Int. Cl. A61K 38/17; A61K 39/00; A61K 39/395; A61K 40/11; A61K 40/31; A61K 40/42; A61K 45/06; A61P 35/00; C07K 14/705; C07K 14/725; C07K 16/28; C12N 5/0783

(52) U.S. Cl. CPC A61K 38/177; A61K 38/1774; A61K 39/3955; A61K 40/11; A61K 40/31; A61K 40/4211; A61K 40/4215; A61K 45/06; A61P 35/00; C07K 14/7051; C07K 14/70521; C07K 14/70578; C07K 16/2803; C07K 16/2878; C12N 5/0636; A61K 2039/505; A61K 2239/29; A61K 2239/38; A61K 2239/48; C07K 2317/53; C07K 2317/565; C07K 2317/622; C07K 2319/02; C07K 2319/03; C07K 2319/30; C07K 2319/33; C12N 2501/2302; C12N 2501/2306; C12N 2501/2307; C12N 2501/2315; C12N 2501/2321

(57) ABSTRACT

The invention provides immune effector cells (for example, T cells, NK cells) that express a chimeric antigen receptor (CAR), and compositions and methods thereof.

### CROSS-REFERENCE TO RELATED APPLICATIONS

This application is a continuation of U.S. application Ser. No. 17/104,983, filed Nov. 25, 2020, now allowed, which claims priority to U.S. Ser. No. 62/940,509, filed on Nov. 26, 2019, the entire contents of each of which are incorporated herein by reference.

### SEQUENCE LISTING

The instant application contains a Sequence Listing which has been submitted electronically in XML format and is hereby incorporated by reference in its entirety. Said XML copy, created on Oct. 17, 2024, is named N2067-716620_SL.xml and is 672,287 bytes in size.

### FIELD OF THE INVENTION

The present invention relates generally to immune effector cells (for example, T cells or NK cells) engineered to express a Chimeric Antigen Receptor (CAR), and compositions and uses thereof.

### BACKGROUND OF THE INVENTION

Adoptive cell transfer (ACT) therapy with T cells, especially with T cells transduced with Chimeric Antigen Receptors (CARs), has shown promise in several hematologic cancer trials. There exists a need for methods and processes to improve production of the CAR-expressing cell therapy product, enhance product quality, and maximize the therapeutic efficacy of the product.

### SUMMARY OF THE INVENTION

In one aspect, this invention features a cell, e.g., an immune cell, e.g., a T cell or NK cell, comprising a first antigen-binding domain and a second antigen-binding domain. In some embodiments, the first antigen-binding domain is an anti-BCMA binding domain. In some embodiments, the anti-BCMA binding domain comprises an anti-BCMA binding sequence disclosed herein, e.g., a CDR, VH, VL, or scFv sequence disclosed in Tables 3-15, 19, 20, 22, 26 and 31. In some embodiments, the second antigen-binding domain is an anti-CD19 binding domain. In some embodiments, the anti-CD19 binding domain comprises an anti-CD19 binding sequence disclosed herein, e.g., a CDR, VH, VL, or scFv sequence disclosed in Tables 2, 19, 22, and 31.

In some embodiments, the present invention provides a cell comprising (a) a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of: (i) SEQ ID NOs: 86, 130, 88, 95, 131, and 132, respectively; (ii) SEQ ID NOs: 44, 45, 84, 54, 55, and 56, respectively; or (iii) SEQ ID NOs: 179, 180, 181, 147, 182, and 183, respectively; and (b) a second antigen-binding domain. In some embodiments, the first antigen-binding domain and the second antigen-binding domain are disposed in two chimeric antigen receptor (CARs).

In some embodiments, the first antigen-binding domain and the second antigen-binding domain are disposed in one CAR. In some embodiments, the HC CDR1, HC CDR2, HC CDR3, LC CDR1, LC CDR2, and LC CDR3 comprise the amino acid sequences of SEQ ID NOs: 86, 130, 88, 95, 131, and 132, respectively. In some embodiments, the HC CDR1, HC CDR2, HC CDR3, LC CDR1, LC CDR2, and LC CDR3 comprise the amino acid sequences of SEQ ID NOs: 86, 87, 88, 95, 96, and 97, respectively. In some embodiments, the HC CDR1, HC CDR2, HC CDR3, LC CDR1, LC CDR2, and LC CDR3 comprise the amino acid sequences of SEQ ID NOs: 86, 109, 88, 95, 114, and 115, respectively. In some embodiments, the HC CDR1, HC CDR2, HC CDR3, LC CDR1, LC CDR2, and LC CDR3 comprise the amino acid sequences of SEQ ID NOs: 86, 109, 88, 95, 114, and 97, respectively. In some embodiments, the VH comprises the amino acid sequence of SEQ ID NO: 93 or 112, or an amino acid sequence having at least about 85%, 90%, 95%, or 99% sequence identity thereto. In some embodiments, the VH is encoded by the nucleic acid sequence of SEQ ID NO: 260, 94 or 113, or a nucleic acid sequence having at least about 85%, 90%, 95%, or 99% sequence identity thereto. In some embodiments, the VL comprises the amino acid sequence of SEQ ID NO: 102, 118, or 124, or an amino acid sequence having at least about 85%, 90%, 95%, or 99% sequence identity thereto. In some embodiments, the VL is encoded by the nucleic acid sequence of SEQ ID NO: 261, 103, 119, or 125, or a nucleic acid sequence having at least about 85%, 90%, 95%, or 99% sequence identity thereto. In some embodiments, the VH and VL comprise the amino acid sequences of SEQ ID NOs: 93 and 102, respectively. In some embodiments, the VH and VL comprise the amino acid sequences of SEQ ID NOs: 112 and 118, respectively. In some embodiments, the VH and VL comprise the amino acid sequences of SEQ ID NOs: 112 and 124, respectively. In some embodiments, the first antigen-binding domain comprises a single-chain fragment variable (scFv) comprising the amino acid sequence of SEQ ID NO: 105, 120, or 126, or an amino acid sequence having at least about 85%, 90%, 95%, or 99% sequence identity thereto. In some embodiments, the first antigen-binding domain is encoded by the nucleic acid sequence of SEQ ID NO: 253, 106, 121, or 127, or a nucleic acid sequence having at least about 85%, 90%, 95%, or 99% sequence identity thereto. In some embodiments, the first antigen-binding domain is disposed in a first CAR. In some embodiments, the first CAR comprises the amino acid sequence of SEQ ID NO: 107, 226, 122, or 128, or an amino acid sequence having at least about 85%, 90%, 95%, or 99% sequence identity thereto. In some embodiments, the first CAR is encoded by the nucleic acid sequence of SEQ ID NO: 259, 258, 108, 123, or 129, or a nucleic acid sequence having at least about 85%, 90%, 95%, or 99% sequence identity thereto.

In some embodiments, the HC CDR1, HC CDR2, HC CDR3, LC CDR1, LC CDR2, and LC CDR3 comprise the amino acid sequences of SEQ ID NOs: 44, 45, 84, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 76, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 46, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 68, 54, 55, and 56, respectively. In some embodiments, the VH comprises the amino acid sequence of SEQ ID NO: 78, 52, or 70, or an amino acid sequence having at least about 85%, 90%, 95%, or 99% sequence identity thereto. In some embodiments, the VH is encoded by the nucleic acid sequence of SEQ ID NO: 79, 53, or 71, or a nucleic acid sequence having at least about 85%, 90%, 95%, or 99% sequence identity thereto. In some embodiments, the VL comprises the amino acid sequence of SEQ ID NO: 61, or an amino acid sequence having at least about 85%, 90%, 95%, or 99% sequence identity thereto. In some embodiments, the VL is encoded by the nucleic acid sequence of SEQ ID NO: 62, or a nucleic acid sequence having at least about 85%, 90%, 95%, or 99% sequence identity thereto. In some embodiments, the VH and VL comprise the amino acid sequences of SEQ ID NOs: 78 and 61, respectively. In some embodiments, the VH and VL comprise the amino acid sequences of SEQ ID NOs: 52 and 61, respectively. In some embodiments, the VH and VL comprise the amino acid sequences of SEQ ID NOs: 70 and 61, respectively. In some embodiments, the first antigen-binding domain comprises a single-chain fragment variable (scFv) comprising the amino acid sequence of SEQ ID NO: 80, 64, or 72, or an amino acid sequence having at least about 85%, 90%, 95%, or 99% sequence identity thereto. In some embodiments, the first antigen-binding domain is encoded by the nucleic acid sequence of SEQ ID NO: 81, 65, or 73, or a nucleic acid sequence having at least about 85%, 90%, 95%, or 99% sequence identity thereto. In some embodiments, the first antigen-binding domain is disposed in a first CAR. In some embodiments, the first CAR comprises the amino acid sequence of SEQ ID NO: 224, 82, 66, or 74, or an amino acid sequence having at least about 85%, 90%, 95%, or 99% sequence identity thereto. In some embodiments, the first CAR is encoded by the nucleic acid sequence of SEQ ID NO: 83, 67, or 75, or a nucleic acid sequence having at least about 85%, 90%, 95%, or 99% sequence identity thereto.

In some embodiments, the HC CDR1, HC CDR2, HC CDR3, LC CDR1, LC CDR2, and LC CDR3 comprise the amino acid sequences of SEQ ID NOs: 179, 180, 181, 147, 182, and 183, respectively. In some embodiments, the HC CDR1, HC CDR2, HC CDR3, LC CDR1, LC CDR2, and LC CDR3 comprise the amino acid sequences of SEQ ID NOs: 137, 138, 139, 147, 148, and 149, respectively. In some embodiments, the HC CDR1, HC CDR2, HC CDR3, LC CDR1, LC CDR2, and LC CDR3 comprise the amino acid sequences of SEQ ID NOs: 160, 161, 162, 147, 170, and 171, respectively. In some embodiments, the VH comprises the amino acid sequence of SEQ ID NO: 145 or 168, or an amino acid sequence having at least about 85%, 90%, 95%, or 99% sequence identity thereto. In some embodiments, the VH is encoded by the nucleic acid sequence of SEQ ID NO: 146 or 169, or a nucleic acid sequence having at least about 85%, 90%, 95%, or 99% sequence identity thereto. In some embodiments, the VL comprises the amino acid sequence of SEQ ID NO: 154 or 173, or an amino acid sequence having at least about 85%, 90%, 95%, or 99% sequence identity thereto. In some embodiments, the VL is encoded by the nucleic acid sequence of SEQ ID NO: 155 or 174, or a nucleic acid sequence having at least about 85%, 90%, 95%, or 99% sequence identity thereto. In some embodiments, the VH and VL comprise the amino acid sequences of SEQ ID NOs: 145 and 154, respectively. In some embodiments, the VH and VL comprise the amino acid sequences of SEQ ID NOs: 168 and 173, respectively. In some embodiments, the first antigen-binding domain comprises a single-chain fragment variable (scFv) comprising the amino acid sequence of SEQ ID NO: 156 or 175, or an amino acid sequence having at least about 85%, 90%, 95%, or 99% sequence identity thereto. In some embodiments, the first antigen-binding domain is encoded by the nucleic acid sequence of SEQ ID NO: 157 or 176, or a nucleic acid sequence having at least about 85%, 90%, 95%, or 99% sequence identity thereto. In some embodiments, the first antigen-binding domain is disposed in a first CAR. In some embodiments, the first CAR comprises the amino acid sequence of SEQ ID NO: 158 or 177, or an amino acid sequence having at least about 85%, 90%, 95%, or 99% sequence identity thereto. In some embodiments, the first CAR is encoded by the nucleic acid sequence of SEQ ID NO: 159 or 178, or a nucleic acid sequence having at least about 85%, 90%, 95%, or 99% sequence identity thereto.

In some embodiments, provided herein is a cell comprising: (a) a first antigen-binding domain which is an anti-BCMA binding domain, wherein the anti-BCMA binding domain comprises: (i) a VH comprising a HC CDR1, HC CDR2, and HC CDR3 of an anti-BCMA sequence listed in Table 20 or 26 and a VL comprising a LC CDR1, LC CDR2, and LC CDR3 of an anti-BCMA sequence listed in Table 20 or 26, wherein the VH and VL are connected by a linker comprising the amino acid sequence of SEQ ID NO: 243; (ii) a VH and VL comprising the amino acid sequences of SEQ ID NOs: 239 and 242, respectively, wherein the VH and VL are connected by a linker comprising the amino acid sequence of SEQ ID NO: 243; or (iii) an scFv comprising the amino acid sequence of SEQ ID NO: 200; and (b) a second antigen-binding domain. In some embodiments, the first antigen-binding domain and the second antigen-binding domain are disposed in two chimeric antigen receptor (CARs). In some embodiments, the first antigen-binding domain and the second antigen-binding domain are disposed in one CAR. In some embodiments, the second antigen-binding domain binds to an antigen chosen from: CD19, CD5, CD10, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, FcRn5, FcRn2, CS-1, CXCR4, 5, 7, IL-7/3R, IL7/4/3R, or IL4R, optionally wherein the B cell antigen is chosen from CD19, CD20, CD22, FcRn5, FcRn2, CS-1, CD138, CD123, CD33, CD34, CLL-1, folate receptor beta, or FLT3. In some embodiments, the second antigen-binding domain binds to CD19. In some embodiments, the second antigen-binding domain binds to an antigen chosen from: EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

In some embodiments, the second antigen-binding domain binds to CD19. In some embodiments, the second antigen-binding domain comprises a HC CDR1, HC CDR2, HC CDR3, LC CDR1, LC CDR2, and/or LC CDR3 of an anti-CD19 sequence listed in Table 19 or Table 22, for example, a HC CDR1, HC CDR2, HC CDR3, LC CDR1, LC CDR2, and LC CDR3 comprising the amino acid sequences of SEQ ID NOs: 295 and 245-249, respectively. In some embodiments, the second antigen-binding domain comprises a VH and/or VL of an anti-CD19 sequence listed in Table 19 or Table 22, for example, a VH and VL comprising the amino acid sequences of SEQ ID NOs: 250 and 251, respectively, or an amino acid sequence having at least about 85%, 90%, 95%, or 99% sequence identity thereto. In some embodiments, the second antigen-binding domain comprises a scFv of an anti-CD19 sequence listed in Table 19 or Table 22, for example, a scFv comprising the amino acid sequence of SEQ ID NO: 211, or an amino acid sequence having at least about 85%, 90%, 95%, or 99% sequence identity thereto. In some embodiments, the second antigen-binding domain is disposed in a second CAR, wherein the CAR comprises a CAR of an anti-CD19 sequence listed in Table 19 or Table 22, for example, a CAR comprising the amino acid sequence of SEQ ID NO: 225 or 229, or an amino acid sequence having at least about 85%, 90%, 95%, or 99% sequence identity thereto.

In some embodiments, the first antigen-binding domain comprises a HC CDR1, HC CDR2, HC CDR3, LC CDR1, LC CDR2, and LC CDR comprising the amino acid sequences of (a) SEQ ID NOs: 86, 87, 88, 95, 96, and 97, respectively; (b) SEQ ID NOs: 44, 45, 76, 54, 55, and 56, respectively; or (c) SEQ ID NOs: 44, 45, 46, 54, 55, and 56, respectively. In some embodiments, the second antigen-binding domain comprises a HC CDR1, HC CDR2, HC CDR3, LC CDR1, LC CDR2, and LC CDR3 comprising the amino acid sequences of SEQ ID NOs: 295 and 245-249, respectively. In some embodiments, the first antigen-binding domain comprises a VH and VL comprising the amino acid sequences of: (a) SEQ ID NOs: 93 and 102, respectively; (b) SEQ ID NOs: 78 and 61, respectively; or (c) SEQ ID NOs: 52 and 61, respectively. In some embodiments, the second antigen-binding domain comprises a VH and VL comprising the amino acid sequences of SEQ ID NOs: 250 and 251, respectively. In some embodiments, the first antigen-binding domain comprises a scFv comprising the amino acid sequence of SEQ ID NO: 105, 80, or 64. In some embodiments, the second antigen-binding domain comprises a scFv comprising the amino acid sequence of SEQ ID NO: 211. In some embodiments, the first antigen-binding domain is encoded by the nucleic acid sequence of SEQ ID NO: 253, 106, 81, or 65. In some embodiments, the second antigen-binding domain is encoded by the nucleic acid sequence of SEQ ID NO: 212.

In some embodiments, the first antigen-binding domain is disposed in a first CAR and the second antigen-binding domain is disposed in a second CAR. In some embodiments, the first CAR further comprises a first transmembrane domain and a first intracellular signaling domain. In some embodiments, the second CAR further comprises a second transmembrane domain and a second intracellular signaling domain.

In some embodiments, the first CAR is encoded by a first nucleic acid sequence and the second CAR is encoded by a second nucleic acid sequence, wherein the first and second nucleic acid sequences are disposed on separate nucleic acid molecules.

In some embodiments, the first CAR is encoded by a first nucleic acid sequence and the second CAR is encoded by a second nucleic acid sequence, wherein the first and second nucleic acid sequences are disposed on a single nucleic acid molecule. In some embodiments, the single nucleic acid molecule comprises the following configuration in a 5′ to 3′ orientation: a nucleic acid sequence encoding the first antigen-binding domain—a nucleic acid sequence encoding a first transmembrane domain—a nucleic acid sequence encoding a first intracellular signaling domain—a nucleic acid sequence encoding a linker—a nucleic acid sequence encoding the second antigen-binding domain—a nucleic acid sequence encoding a second transmembrane domain—a nucleic acid sequence encoding a second intracellular signaling domain. In some embodiments, the single nucleic acid molecule comprises the following configuration in a 5′ to 3′ orientation: a nucleic acid sequence encoding the second antigen-binding domain—a nucleic acid sequence encoding a second transmembrane domain—a nucleic acid sequence encoding a second intracellular signaling domain—a nucleic acid sequence encoding a linker—a nucleic acid sequence encoding the first antigen-binding domain—a nucleic acid sequence encoding a first transmembrane domain—a nucleic acid sequence encoding a first intracellular signaling domain. In some embodiments, the linker comprises a self-cleavage site. In some embodiments, the linker comprises a P2A site, a T2A site, an E2A site, or an F2A site. In some embodiments, the linker comprises a P2A site. In some embodiments, the linker is encoded by the nucleic acid sequence of SEQ ID NO: 209, or a nucleic acid sequence having at least about 85%, 90%, 95%, or 99% sequence identity thereto. In some embodiments, the linker comprises the amino acid sequence of SEQ ID NO: 208, or an amino acid sequence having at least about 85%, 90%, 95%, or 99% sequence identity thereto. In some embodiments, the single nucleic acid molecule comprises the nucleic acid sequence of SEQ ID NO: 215, 217, 219, 221, or 223, or a nucleic acid sequence having at least about 85%, 90%, 95%, or 99% sequence identity thereto. In some embodiments, the single nucleic acid molecule encodes the amino acid sequence of SEQ ID NO: 214, 216, 218, 220, or 222, or an amino acid sequence having at least about 85%, 90%, 95%, or 99% sequence identity thereto.

In some embodiments, the first antigen-binding domain and the second antigen-binding domain are disposed in one CAR, wherein the CAR further comprises a transmembrane domain and an intracellular signaling domain. In some embodiments, the first antigen-binding domain comprises a first VH (VH1) and a first VL (VL1) and the second antigen-binding domain comprises a second VH (VH2) and a second VL (VL2). In some embodiments, the VH1, VL1, VH2, and VL2 are arranged in the following configuration from the N-terminus to the C-terminus: VH2—optionally linker 1 (“L1”)-VL1—optionally linker 2 (“L2”)-VH1—optionally linker 3 (“L3”)-VL2. In some embodiments, the VH1, VL1, VH2, and VL2 are arranged in the following configuration from the N-terminus to the C-terminus: VH1—optionally L1-VH2—optionally L2-VL2—optionally L3-VL1. In some embodiments, the VH1, VL1, VH2, and VL2 are arranged in the following configuration from the N-terminus to the C-terminus: VL2—optionally L1-VL1—optionally L2-VH1—optionally L3-VH2. In some embodiments, the VH1, VL1, VH2, and VL2 are arranged in the following configuration from the N-terminus to the C-terminus: VL2—optionally L1-VH1—optionally L2-VL1—optionally L3-VH2. In some embodiments, the VH1, VL1, VH2, and VL2 are arranged in the following configuration from the N-terminus to the C-terminus: VH2—optionally L1-VH1—optionally L2-VL1—optionally L3-VL2. In some embodiments, the VH1, VL1, VH2, and VL2 are arranged in the following configuration from the N-terminus to the C-terminus: VL1—optionally L1-VH2—optionally L2-VL2—optionally L3-VH1. In some embodiments, the VH1, VL1, VH2, and VL2 are arranged in the following configuration from the N-terminus to the C-terminus: VL1—optionally L1-VL2—optionally L2-VH2—optionally L3-VH1. In some embodiments, the VH1, VL1, VH2, and VL2 are arranged in the following configuration from the N-terminus to the C-terminus: VH1—optionally L1-VL2—optionally L2-VH2—optionally L3-VL1. In some embodiments, the VH1 and VL1 comprise the amino acid sequences of SEQ ID NOs: 93 and 102, respectively (or an amino acid sequence having at least about 85%, 90%, 95%, or 99% sequence identity thereto). In some embodiments, the VH1 and VL1 comprise the amino acid sequences of SEQ ID NOs: 333 and 334, respectively (or an amino acid sequence having at least about 85%, 90%, 95%, or 99% sequence identity thereto). In some embodiments, the VH1 and VL1 comprise the amino acid sequences of SEQ ID NOs: 78 and 61, respectively (or an amino acid sequence having at least about 85%, 90%, 95%, or 99% sequence identity thereto). In some embodiments, the VH1 and VL1 comprise the amino acid sequences of SEQ ID NOs: 335 and 336, respectively (or an amino acid sequence having at least about 85%, 90%, 95%, or 99% sequence identity thereto). In some embodiments, the VH2 and VL2 comprise the amino acid sequences of SEQ ID NOs: 250 and 251, respectively (or an amino acid sequence having at least about 85%, 90%, 95%, or 99% sequence identity thereto). In some embodiments, the VH2 and VL2 comprise the amino acid sequences of SEQ ID NOs: 331 and 332, respectively (or an amino acid sequence having at least about 85%, 90%, 95%, or 99% sequence identity thereto). In some embodiments, L1 or L3 comprises the amino acid sequence of SEQ ID NO: 5 (or an amino acid sequence having at least about 85%, 90%, 95%, or 99% sequence identity thereto). In some embodiments, L2 comprises the amino acid sequence of SEQ ID NO: 63 (or an amino acid sequence having at least about 85%, 90%, 95%, or 99% sequence identity thereto). In some embodiments, the CAR comprises an amino acid sequence selected from the group consisting of SEQ ID NOs: 321-330, or an amino acid sequence having at least 80, 85, 90, 95, or 99% identity thereto. In some embodiments, the CAR comprises an amino acid sequence selected from the group consisting of SEQ ID NOs: 339-348, or an amino acid sequence having at least 80, 85, 90, 95, or 99% identity thereto.

In some embodiments, the CAR is encoded by a nucleic acid molecule comprising the following configuration in a 5′ to 3′ orientation: a nucleic acid sequence encoding the first antigen-binding domain—optionally a nucleic acid sequence encoding a linker—a nucleic acid sequence encoding the second antigen-binding domain—a nucleic acid sequence encoding a transmembrane domain—a nucleic acid sequence encoding an intracellular signaling domain. In some embodiments, the CAR is encoded by a nucleic acid molecule comprising the following configuration in a 5′ to 3′ orientation: a nucleic acid sequence encoding the second antigen-binding domain—optionally a nucleic acid sequence encoding a linker—a nucleic acid sequence encoding the first antigen-binding domain—a nucleic acid sequence encoding a transmembrane domain—a nucleic acid sequence encoding an intracellular signaling domain.

In some embodiments, the CAR comprises the following configuration in an N- to C-orientation: the first antigen-binding domain—optionally a linker—the second antigen-binding domain—a transmembrane domain—an intracellular signaling domain. In some embodiments, the CAR comprises the following configuration in an N- to C-orientation: the second antigen-binding domain—optionally a linker—the first antigen-binding domain—a transmembrane domain—an intracellular signaling domain.

In some embodiments, the first antigen-binding domain or second antigen-binding domain comprises a VH and a VL. In some embodiments, the VH and VL are connected by a linker. In some embodiments, the linker comprises the amino acid sequence of SEQ ID NO: 5, 63, 104, or 243, or an amino acid sequence having at least about 85%, 90%, 95%, or 99% sequence identity thereto.

In some embodiments, the transmembrane domain, first transmembrane domain, or second transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 or CD154. In some embodiments, the transmembrane domain, first transmembrane domain, or second transmembrane domain comprises the amino acid sequence of SEQ ID NO: 6, or an amino acid sequence having at least about 85%, 90%, 95%, or 99% sequence identity thereto. In some embodiments, the transmembrane domain, first transmembrane domain, or second transmembrane domain is encoded by the nucleic acid sequence of SEQ ID NO: 17, or a nucleic acid sequence having at least about 85%, 90%, 95%, or 99% sequence identity thereto.

In some embodiments, the first antigen-binding domain or second antigen-binding domain is connected to the transmembrane domain, first transmembrane domain, or second transmembrane domain by a hinge region (e.g., a first or second hinge region). In some embodiments, the hinge region comprises the amino acid sequence of SEQ ID NO: 2, 3, or 4, or an amino acid sequence having at least about 85%, 90%, 95%, or 99% sequence identity thereto. In some embodiments, the hinge region is encoded by the nucleic acid sequence of SEQ ID NO: 13, 14, or 15, or a nucleic acid sequence having at least about 85%, 90%, 95%, or 99% sequence identity thereto. In some embodiments, the hinge region and the transmembrane domain comprise the amino acid sequence of SEQ ID NO: 202, or an amino acid sequence having at least about 85%, 90%, 95%, or 99% sequence identity thereto. In some embodiments, the hinge region and the transmembrane domain are encoded by the nucleic acid sequence of SEQ ID NO: 203 or 213, or a nucleic acid sequence having at least about 85%, 90%, 95%, or 99% sequence identity thereto.

In some embodiments, the intracellular signaling domain, first intracellular signaling domain, or second intracellular signaling domain comprises a primary signaling domain (e.g., a first or second primary signaling domain). In some embodiments, the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d. In some embodiments, the primary signaling domain comprises the amino acid sequence of SEQ ID NO: 9 or 10, or an amino acid sequence having at least about 85%, 90%, 95%, or 99% sequence identity thereto. In some embodiments, the primary signaling domain is encoded by the nucleic acid sequence of SEQ ID NO: 20, 21, or 205, or a nucleic acid sequence having at least about 85%, 90%, 95%, or 99% sequence identity thereto. In some embodiments, the intracellular signaling domain, first intracellular signaling domain, or second intracellular signaling domain comprises a costimulatory signaling domain (e.g., a first or second costimulatory signaling domain). In some embodiments, the costimulatory signaling domain comprises a functional signaling domain derived from a MHC class I molecule, a TNF receptor protein, an Immunoglobulin-like protein, a cytokine receptor, an integrin, a signalling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, the costimulatory signaling domain comprises the amino acid sequence of SEQ ID NO: 7, or an amino acid sequence having at least about 85%, 90%, 95%, or 99% sequence identity thereto. In some embodiments, the costimulatory signaling domain is encoded by the nucleic acid sequence of SEQ ID NO: 18 or 204, or a nucleic acid sequence having at least about 85%, 90%, 95%, or 99% sequence identity thereto. In some embodiments, the intracellular signaling domain, first intracellular signaling domain, or second intracellular signaling domain comprises a functional signaling domain derived from 4-1BB and a functional signaling domain derived from CD3 zeta. In some embodiments, the intracellular signaling domain, first intracellular signaling domain, or second intracellular signaling domain comprises the amino acid sequence of SEQ ID NO: 7 (or an amino acid sequence having at least about 85%, 90%, 95%, or 99% sequence identity thereto) and the amino acid sequence of SEQ ID NO: 9 or 10 (or an amino acid sequence having at least about 85%, 90%, 95%, or 99% sequence identity thereto). In some embodiments, the intracellular signaling domain, first intracellular signaling domain, or second intracellular signaling domain comprises the amino acid sequence of SEQ ID NO: 7 and the amino acid sequence of SEQ ID NO: 9 or 10.

In some embodiments, the CAR, first CAR, or second CAR further comprises a leader sequence (e.g., a first or second leader sequence). In some embodiments, the leader sequence comprises the amino acid sequence of SEQ ID NO: 1, or an amino acid sequence having at least about 85%, 90%, 95%, or 99% sequence identity thereto. In some embodiments, the leader sequence is encoded by the nucleic acid sequence of SEQ ID NO: 199 or 210, or a nucleic acid sequence having at least about 85%, 90%, 95%, or 99% sequence identity thereto.

In some embodiments, the first leader sequence and the second leader sequence are encoded by different nucleic acid sequences (e.g., differ by at least 5%, 10%, 20%, 30%, 40%, 50%, 60%, 70%, 80%, 85%, 90%, 95%, or 100%). In some embodiments, the first hinge region and the second hinge region are encoded by different nucleic acid sequences (e.g., differ by at least 5%, 10%, 20%, 30%, 40%, 50%, 60%, 70%, 80%, 85%, 90%, 95%, or 100%). In some embodiments, the first transmembrane domain and the second transmembrane domain are encoded by different nucleic acid sequences (e.g., differ by at least 5%, 10%, 20%, 30%, 40%, 50%, 60%, 70%, 80%, 85%, 90%, 95%, or 100%). In some embodiments, the first intracellular signaling domain and the second intracellular signaling domain are encoded by different nucleic acid sequences (e.g., differ by at least 5%, 10%, 20%, 30%, 40%, 50%, 60%, 70%, 80%, 85%, 90%, 95%, or 100%). In some embodiments, the first primary signaling domain and the second primary signaling domain are encoded by different nucleic acid sequences (e.g., differ by at least 5%, 10%, 20%, 30%, 40%, 50%, 60%, 70%, 80%, 85%, 90%, 95%, or 100%). In some embodiments, the first costimulatory signaling domain and the second costimulatory signaling domain are encoded by different nucleic acid sequences (e.g., differ by at least 5%, 10%, 20%, 30%, 40%, 50%, 60%, 70%, 80%, 85%, 90%, 95%, or 100%). In some embodiments, the first leader sequence and the second leader sequence comprise the same amino acid sequence (e.g., the first leader sequence and the second leader sequence comprise the amino acid sequence of SEQ ID NO: 1, or an amino acid sequence having at least about 85%, 90%, 95%, or 99% sequence identity thereto). In some embodiments, the first leader sequence and the second leader sequence comprise different amino acid sequences. In some embodiments, the first hinge region and the second hinge region comprise the same amino acid sequence (e.g., the first hinge region and the second hinge region comprise the amino acid sequence of SEQ ID NO: 2, or an amino acid sequence having at least about 85%, 90%, 95%, or 99% sequence identity thereto). In some embodiments, the first hinge region and the second hinge region comprise different amino acid sequences. In some embodiments, the first transmembrane domain and the second transmembrane domain comprise the same amino acid sequence (e.g., the first transmembrane domain and the second transmembrane domain comprise the amino acid sequence of SEQ ID NO: 6, or an amino acid sequence having at least about 85%, 90%, 95%, or 99% sequence identity thereto). In some embodiments, the first transmembrane domain and the second transmembrane domain comprise different amino acid sequences. In some embodiments, the first intracellular signaling domain and the second intracellular signaling domain comprise the same amino acid sequence. In some embodiments, the first intracellular signaling domain and the second intracellular signaling domain comprise different amino acid sequences. In some embodiments, the first primary signaling domain and the second primary signaling domain comprise the same amino acid sequence (e.g., the first primary signaling domain and the second primary signaling domain comprise the amino acid sequence of SEQ ID NO: 10, or an amino acid sequence having at least about 85%, 90%, 95%, or 99% sequence identity thereto). In some embodiments, the first primary signaling domain and the second primary signaling domain comprise different amino acid sequences. In some embodiments, the first costimulatory signaling domain and the second costimulatory signaling domain comprise the same amino acid sequence (e.g., the first costimulatory signaling domain and the second costimulatory signaling domain comprise the amino acid sequence of SEQ ID NO: 7, or an amino acid sequence having at least about 85%, 90%, 95%, or 99% sequence identity thereto). In some embodiments, the first costimulatory signaling domain and the second costimulatory signaling domain comprise different amino acid sequences (e.g., the first and second costimulatory signaling domains comprise a 4-1BB costimulatory domain sequence and a CD28 costimulatory domain sequence, respectively; or comprise a CD28 costimulatory domain sequence and a 4-1BB costimulatory domain sequence, respectively). In some embodiments, the first leader sequence and the second leader sequence are encoded by nucleic acid sequences comprising SEQ ID NOs: 199 and 210, respectively (or a nucleic acid sequence having at least about 85%, 90%, 95%, or 99% sequence identity thereto). In some embodiments, the first leader sequence and the second leader sequence are encoded by nucleic acid sequences comprising SEQ ID NOs: 210 and 199, respectively (or a nucleic acid sequence having at least about 85%, 90%, 95%, or 99% sequence identity thereto). In some embodiments, the first hinge region and the second hinge region are encoded by nucleic acid sequences comprising SEQ ID NOs: 337 and 13, respectively (or a nucleic acid sequence having at least about 85%, 90%, 95%, or 99% sequence identity thereto). In some embodiments, the first hinge region and the second hinge region are encoded by nucleic acid sequences comprising SEQ ID NOs: 13 and 337, respectively (or a nucleic acid sequence having at least about 85%, 90%, 95%, or 99% sequence identity thereto). In some embodiments, the first transmembrane domain and the second transmembrane domain are encoded by nucleic acid sequences comprising SEQ ID NOs: 338 and 17, respectively (or a nucleic acid sequence having at least about 85%, 90%, 95%, or 99% sequence identity thereto). In some embodiments, the first transmembrane domain and the second transmembrane domain are encoded by nucleic acid sequences comprising SEQ ID NOs: 17 and 338, respectively (or a nucleic acid sequence having at least about 85%, 90%, 95%, or 99% sequence identity thereto). In some embodiments, the first costimulatory signaling domain and the second costimulatory signaling domain are encoded by nucleic acid sequences comprising SEQ ID NOs: 204 and 18, respectively (or a nucleic acid sequence having at least about 85%, 90%, 95%, or 99% sequence identity thereto). In some embodiments, the first costimulatory signaling domain and the second costimulatory signaling domain are encoded by nucleic acid sequences SEQ ID NOs: 18 and 204, respectively (or a nucleic acid sequence having at least about 85%, 90%, 95%, or 99% sequence identity thereto). In some embodiments, the first primary signaling domain and the second primary signaling domain are encoded by nucleic acid sequences comprising SEQ ID NOs: 205 and 21, respectively (or a nucleic acid sequence having at least about 85%, 90%, 95%, or 99% sequence identity thereto). In some embodiments, the first primary signaling domain and the second primary signaling domain are encoded by nucleic acid sequences comprising SEQ ID NOs: 21 and 205, respectively (or a nucleic acid sequence having at least about 85%, 90%, 95%, or 99% sequence identity thereto).

In some embodiments, the CAR, first CAR, or second CAR is encoded by a nucleic acid molecule comprising a woodchuck hepatitis post-transcriptional regulatory element (WPRE).

In some embodiments, provided herein is a nucleic acid molecule comprising: (a) a first nucleic acid sequence encoding a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of: (i) SEQ ID NOs: 86, 130, 88, 95, 131, and 132, respectively; (ii) SEQ ID NOs: 44, 45, 84, 54, 55, and 56, respectively; or (iii) SEQ ID NOs: 179, 180, 181, 147, 182, and 183, respectively; and (b) a second nucleic acid sequence encoding a second antigen-binding domain.

In some embodiments, the isolated nucleic acid molecule comprises a first nucleic acid molecule and a second nucleic acid molecule, which are separate nucleic acid molecules, and wherein the first nucleic acid sequence is disposed on the first nucleic acid molecule and the second nucleic acid sequence is disposed on the second nucleic acid molecule.

In some embodiments, provided herein is a nucleic acid molecule comprising: (a) a first nucleic acid sequence encoding a first antigen-binding domain which is an anti-BCMA binding domain, wherein the anti-BCMA binding domain comprises: (i) a VH comprising a HC CDR1, HC CDR2, and HC CDR3 of an anti-BCMA sequence listed in Table 20 or 26 and a VL comprising a LC CDR1, LC CDR2, and LC CDR3 of an anti-BCMA sequence listed in Table 20 or 26, wherein the VH and VL are connected by a linker comprising the amino acid sequence of SEQ ID NO: 243; (ii) a VH and VL comprising the amino acid sequences of SEQ ID NOs: 239 and 242, respectively, wherein the VH and VL are connected by a linker comprising the amino acid sequence of SEQ ID NO: 243; or (iii) an scFv comprising the amino acid sequence of SEQ ID NO: 200; and (b) a second nucleic acid sequence encoding a second antigen-binding domain.

In some embodiments, provided herein is a nucleic acid molecule comprising a first nucleic acid sequence encoding a first CAR and a second nucleic acid sequence encoding a second CAR, wherein the first CAR comprises a first antigen-binding domain which is an anti-BCMA binding domain, a first transmembrane domain, and a first intracellular signaling domain, and wherein the second CAR comprises a second antigen-binding domain which is an anti-CD19 binding domain, a second transmembrane domain, and a second intracellular signaling domain, wherein (i) the first antigen-binding domain comprises a HC CDR1, HC CDR2, HC CDR3, LC CDR1, LC CDR2, and LC CDR comprising the amino acid sequences of SEQ ID NOs: 86, 87, 88, 95, 96, and 97, respectively, and the second antigen-binding domain comprises a HC CDR1, HC CDR2, HC CDR3, LC CDR1, LC CDR2, and LC CDR comprising the amino acid sequences of SEQ ID NOs: 295 and 245-249, respectively; (ii) the first antigen-binding domain comprises a VH and VL comprising the amino acid sequences of SEQ ID NOs: 93 and 102, respectively, and the second antigen-binding domain comprises a VH and VL comprising the amino acid sequences of SEQ ID NOs: 250 and 251, respectively; (iii) the first antigen-binding domain comprises an scFv comprising the amino acid sequence of SEQ ID NO: 105, and the second antigen-binding domain comprises an scFv comprising the amino acid sequence of SEQ ID NO: 211; (iv) the first CAR comprises the amino acid sequence of SEQ ID NO: 107 or 226 and the second CAR comprises the amino acid sequence of SEQ ID NO: 225 or 229; or (v) the isolated nucleic acid molecule comprises the nucleic acid sequence of SEQ ID NO: 271.

In some embodiments, provided herein is a nucleic acid molecule comprising a first nucleic acid sequence encoding a first CAR and a second nucleic acid sequence encoding a second CAR, wherein the first CAR comprises a first antigen-binding domain which is an anti-BCMA binding domain, a first transmembrane domain, and a first intracellular signaling domain, and wherein the second CAR comprises a second antigen-binding domain which is an anti-CD19 binding domain, a second transmembrane domain, and a second intracellular signaling domain, wherein: (i) the first antigen-binding domain comprises a HC CDR1, HC CDR2, HC CDR3, LC CDR1, LC CDR2, and LC CDR comprising the amino acid sequences of SEQ ID NOs: 44, 45, 76, 54, 55, and 56, respectively, the second antigen-binding domain comprises a HC CDR1, HC CDR2, HC CDR3, LC CDR1, LC CDR2, and LC CDR3 comprising the amino acid sequences of SEQ ID NOs: 295 and 245-249, respectively; (ii) the first antigen-binding domain comprises a VH and VL comprising the amino acid sequences of SEQ ID NOs: 78 and 61, respectively, and the second antigen-binding domain comprises a VH and VL comprising the amino acid sequences of SEQ ID NOs: 250 and 251, respectively; (iii) the first antigen-binding domain comprises an scFv comprising the amino acid sequence of SEQ ID NO: 80, and the second antigen-binding domain comprises an scFv comprising the amino acid sequence of SEQ ID NO: 211; (iv) the first CAR comprises the amino acid sequence of SEQ ID NO: 82 or 224 and the second CAR comprises the amino acid sequence of SEQ ID NO: 225 or 229; or (v) the isolated nucleic acid molecule comprises the nucleic acid sequence of SEQ ID NO: 215.

In some embodiments, provided herein is a polypeptide molecule encoded by a nucleic acid molecule disclosed herein.

In some embodiments, provided herein is a CAR, wherein the CAR comprises: (a) a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of: (i) SEQ ID NOs: 86, 130, 88, 95, 131, and 132, respectively; (ii) SEQ ID NOs: 44, 45, 84, 54, 55, and 56, respectively; or (iii) SEQ ID NOs: 179, 180, 181, 147, 182, and 183, respectively; and (b) a second antigen-binding domain.

In some embodiments, provided herein is a CAR, wherein the CAR comprises: (a) a first antigen-binding domain which is an anti-BCMA binding domain, wherein the anti-BCMA binding domain comprises: (i) a VH comprising a HC CDR1, HC CDR2, and HC CDR3 of an anti-BCMA sequence listed in Table 20 or 26 and a VL comprising a LC CDR1, LC CDR2, and LC CDR3 of an anti-BCMA sequence listed in Table 20 or 26, wherein the VH and VL are connected by a linker comprising the amino acid sequence of SEQ ID NO: 243; (ii) a VH and VL comprising the amino acid sequences of SEQ ID NOs: 239 and 242, respectively, wherein the VH and VL are connected by a linker comprising the amino acid sequence of SEQ ID NO: 243; or (iii) an scFv comprising the amino acid sequence of SEQ ID NO: 200; and (b) a second antigen-binding domain.

In some embodiments, provided herein is a vector comprising a nucleic acid molecule disclosed herein or a nucleic acid molecule encoding a CAR disclosed herein. In some embodiments, the vector is chosen from a DNA vector, a RNA vector, a plasmid, a lentivirus vector, an adenoviral vector, or a retrovirus vector. In some embodiments, the vector comprises an EF-1 promoter comprising the nucleic acid sequence of SEQ ID NO: 11.

In some embodiments, provided herein is a cell comprising a nucleic acid molecule disclosed herein, a nucleic acid molecule encoding a CAR disclosed herein, a polypeptide disclosed herein, a CAR disclosed herein, or a vector disclosed herein. In some embodiments, the cell is a T cell or an NK cell.

In some embodiments, disclosed herein is a method of making a cell comprising transducing a cell with a vector disclosed herein, optionally wherein the cell is a T cell or NK cell. In some embodiments, disclosed herein is a method of making an RNA-engineered cell comprising introducing an in vitro transcribed RNA or synthetic RNA into a cell, wherein the RNA comprises a nucleic acid molecule disclosed herein, a nucleic acid molecule encoding a CAR disclosed herein. In some embodiments, the cell is a T cell or NK cell.

In some embodiments, disclosed herein is a method of making a population of cells (for example, T cells) that express a chimeric antigen receptor (CAR), the method comprising: (i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a nucleic acid molecule disclosed herein, or a nucleic acid molecule encoding a CAR disclosed herein, thereby providing a population of cells (for example, T cells) comprising the nucleic acid molecule, and (iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
   step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the nucleic acid molecule in step (ii) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

In some embodiments, disclosed herein is a method of making a population of cells (for example, T cells) that express a chimeric antigen receptor (CAR), the method comprising: (1) contacting a population of cells (for example, T cells, for example, T cells isolated from a frozen leukapheresis product) with a cytokine chosen from IL-2, IL-7, IL-15 (for example, hetIL-15 (IL15/sIL-15Ra)), IL-21, IL-6 (for example, IL-6/sIL-6Ra), or a combination thereof, (2) contacting the population of cells (for example, T cells) with a nucleic acid molecule disclosed herein, or a nucleic acid molecule encoding a CAR disclosed herein, thereby providing a population of cells (for example, T cells) comprising the nucleic acid molecule, and (3) harvesting the population of cells (for example, T cells) for storage (for example, reformulating the population of cells in cryopreservation media) or administration, wherein:
(a) step (2) is performed together with step (1) or no later than 5 hours after the beginning of step (1), for example, no later than 1, 2, 3, 4, or 5 hours after the beginning of step (1), and
   step (3) is performed no later than 26 hours after the beginning of step (1), for example, no later than 22, 23, or 24 hours after the beginning of step (1), for example, no later than 24 hours after the beginning of step (1), or
(b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1),
optionally wherein the nucleic acid molecule in step (2) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

In some embodiments, disclosed herein is a population of cells engineered to express a CAR (“a population of CAR-expressing cells”), said population comprising: (a) about the same percentage of naïve cells, for example, naïve T cells, for example, CD45RO− CCR7+ T cells, as compared to the percentage of naïve cells, for example, naïve T cells, for example, CD45RO− CCR7+ cells, in the same population of cells prior to being engineered to express the CAR; (b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to express the CAR; (c) an increased percentage of naïve cells, for example, naïve T cells, for example, CD45RO− CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to express the CAR; (d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to express the CAR; (g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; (h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; or (i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR. In some embodiments, the population comprises a cell disclosed herein. In some embodiments, the population comprises a cell comprising a dual CAR or diabody CAR disclosed herein. In some embodiments, the population comprises a cell comprising (a) a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of: (i) SEQ ID NOs: 86, 130, 88, 95, 131, and 132, respectively; (ii) SEQ ID NOs: 44, 45, 84, 54, 55, and 56, respectively; or (iii) SEQ ID NOs: 179, 180, 181, 147, 182, and 183, respectively; and (b) a second antigen-binding domain.

In some embodiments, disclosed herein is a pharmaceutical composition comprising a cell disclosed herein or a population of cells disclosed herein, and a pharmaceutically acceptable carrier.

In some embodiments, the population of cells is made by a method disclosed herein. In some embodiments, the population comprises:
(a) a first population of cells comprising an anti-BCMA CAR but not an anti-CD19 CAR;
(b) a second population of cells comprising an anti-CD19 CAR but not an anti-BCMA CAR; and
(c) a third population of cells comprising both an anti-BCMA CAR and an anti-CD19 CAR.

### In Some Embodiments:

(i) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined;
(ii) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined; and/or
(iii) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the population.

In some embodiments, the population further comprises a fourth population of cells that do not comprise a CAR.

### In Some Embodiments:

(i) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined;
(ii) the total number of viable cells in the second population is less than or equal to: about 45% to about 50% (e.g., about 47%); about 50 to about 55% (e.g., about 53%); about 60% to about 65% (e.g., about 63%); or about 80 to about 85% (e.g., about 82%) of the total number of viable cells in the first and third populations combined.

In some embodiments, disclosed herein is a method of providing an anti-tumor immunity in a subject comprising administering to the subject an effective amount of a cell disclosed herein, a population of cells disclosed herein, or a pharmaceutical composition disclosed herein. In some embodiments, disclosed herein is a method of treating a subject having a disease associated with expression of BCMA comprising administering to the subject an effective amount of a cell disclosed herein, a population of cells disclosed herein, or a pharmaceutical composition disclosed herein. In some embodiments, the disease associated with BCMA expression is: (i) a cancer or malignancy, or a precancerous condition chosen from one or more of a myelodysplasia, a myelodysplastic syndrome or a preleukemia, or (ii) a non-cancer related indication associated with expression of BCMA. In some embodiments, the disease is a hematologic cancer or a solid cancer. In some embodiments, the disease is chosen from: acute leukemia, B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell- or large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, prostate cancer (e.g., castrate-resistant or therapy-resistant prostate cancer, or metastatic prostate cancer), pancreatic cancer, lung cancer, a plasma cell proliferative disorder (e.g., asymptomatic myeloma (smoldering multiple myeloma or indolent myeloma), monoclonal gammapathy of undetermined significance (MGUS), Waldenstrom's macroglobulinemia, plasmacytoma (e.g., plasma cell dyscrasia, solitary myeloma, solitary plasmacytoma, extramedullary plasmacytoma, and multiple plasmacytoma), systemic amyloid light chain amyloidosis, or POEMS syndrome (also known as Crow-Fukase syndrome, Takatsuki disease, and PEP syndrome)), or a combination thereof. In some embodiments, the disease is multiple myeloma.

In some embodiments, the population of cells or pharmaceutical composition is administered to the subject at a dose of about 1×10⁶ to about 1×10⁸ (e.g., about 2×10⁶ to about 5×10⁷, about 5×10⁶ to about 2×10⁷, about 1×10⁶ to about 1×10⁷, about 1×10⁷ to about 1×10⁸, about 1×10⁶ to about 3×10⁶, about 2×10⁶ to about 4×10⁶, about 3×10⁶ to about 5×10⁶, about 4×10⁶ to about 6×10⁶, about 5×10⁶ to about 7×10⁶, about 6×10⁶ to about 8×10⁶, about 7×10⁶ to about 9×10⁶, about 8×10⁶ to about 1×10⁷, about 9×10⁶ to about 2×10⁷, about 1×10⁷ to about 3×10⁷, about 2×10⁷ to about 4×10⁷, about 3×10⁷ to about 5×10⁷, about 4×10⁷ to about 6×10⁷, about 5×10⁷ to about 7×10⁷, about 6×10⁷ to about 8×10⁷, about 7×10⁷ to about 9×10⁷, about 8×10⁷ to about 1×10⁸, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸) CAR-positive viable cells (e.g., BCMA CAR+ T cells). In some embodiments, the population of cells or pharmaceutical composition is administered to the subject at a dose of about 5×10⁶ to about 2×10⁷ CAR-positive viable cells (e.g., BCMA CAR+ T cells).

In some embodiments, the population of cells or pharmaceutical composition is administered to the subject in one or more (e.g., 2, 3, 4, or more) doses. In some embodiments, the population of cells or pharmaceutical composition is administered to the subject in two doses. In some embodiments, the one or more doses comprises a first dose and a second dose, wherein the number of CAR-positive viable cells (e.g., BCMA CAR+ T cells) in the first dose is greater than, equal to, or less than the number of CAR-positive viable cells (e.g., BCMA CAR+ T cells) in the second dose.

In some embodiments, the one or more doses comprise a first dose and a second dose, wherein:
(a) the first dose comprises about 1×10⁶ to about 1×10⁷(e.g., about 2×10⁶ to about 8×10⁶, about 4×10⁶ to about 6×10⁶, about 1×10⁶ to about 5×10⁶, about 5×10⁶ to about 1×10⁷, about 1×10⁶ to about 3×10⁶, about 2×10⁶ to about 4×10⁶, about 3×10⁶ to about 5×10⁶, about 4×10⁶ to about 6×10⁶, about 5×10⁶ to about 7×10⁶, about 6×10⁶ to about 8×10⁶, about 7×10⁶ to about 9×10⁶, about 8×10⁶ to about 1×10⁷, about 1×10⁶, about 2×10⁶, about 3×10⁶, about 4×10⁶, about 5×10⁶, about 6×10⁶, about 7×10⁶, about 8×10⁶, about 9×10⁶, or about 1×10⁷) viable CAR-positive cells (e.g., BCMA CAR+ T cells);
(b) the second dose comprises about 1×10⁷ to about 1×10⁸ (e.g., about 2×10⁷ to about 8×10⁷, about 4×10⁷ to about 6×10⁷, about 1×10⁷ to about 5×10⁷, about 5×10⁷ to about 1×10⁸, about 1×10⁷ to about 3×10⁷, about 2×10⁷ to about 4×10⁷, about 3×10⁷ to about 5×10⁷, about 4×10⁷ to about 6×10⁷, about 5×10⁷ to about 7×10⁷, about 6×10⁷ to about 8×10⁷, about 7×10⁷ to about 9×10⁷, about 8×10⁷ to about 1×10⁸, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸) CAR-positive viable cells (e.g., BCMA CAR+ T cells);
(c) the number of CAR-positive viable cells (e.g., BCMA CAR+ T cells) in the first dose is no more than 1/X, wherein X is 1, 2, 3, 4, 5, 6, 7, 8, 9, 10, 20, 30, 40, 50, 60, 70, 80, 90, or 100, of the number of CAR-positive viable cells (e.g., BCMA CAR+ T cells) in the second dose; and/or
(d) the number of CAR-positive viable cells (e.g., BCMA CAR+ T cells) in the first dose is between about 1% and 100% (e.g., between about 10% and about 90%, between about 20% and about 80%, between about 30% and about 70%, between about 40% and about 60%, between about 10% and about 50%, between about 50% and about 90%, between about 10% and about 30%, between about 20% and about 40%, between about 30% and about 50%, between about 50% and about 70%, between about 60% and about 80%, or between about 70% and about 90%) of the number of CAR-positive viable cells (e.g., BCMA CAR+ T cells) in the second dose.

In some embodiments, the first dose comprises about 5×10⁶ viable CAR-positive cells (e.g., BCMA CAR+ T cells). In some embodiments, the second dose comprises about 1×10⁷ or about 2×10⁷ viable CAR-positive cells (e.g., BCMA CAR+ T cells).

In some embodiments, the method further comprises administering to the subject a second therapeutic agent. In some embodiments, the second therapeutic agent is chosen from: (i) a PD-1 inhibitor, optionally wherein the PD-1 inhibitor is selected from the group consisting of PDR001, Nivolumab, Pembrolizumab, Pidilizumab, MEDI0680, REGN2810, TSR-042, PF-06801591, and AMP-224; (ii) a PD-L1 inhibitor, optionally wherein the PD-L1 inhibitor is selected from the group consisting of FAZ053, Atezolizumab, Avelumab, Durvalumab, and BMS-936559; (iii) a LAG-3 inhibitor, optionally wherein the LAG-3 inhibitor is selected from the group consisting of LAG525, BMS-986016, TSR-033, MK-4280 and REGN3767; (iv) a TIM-3 inhibitor, optionally wherein the TIM-3 inhibitor is selected from the group consisting of MBG453, TSR-022, and LY3321367; (v) a CTLA-4 inhibitor, optionally wherein the CTLA-4 inhibitor is Ipilimumab or Tremelimumab; (vi) an interleukin-15 (IL-15) polypeptide, an interleukin-15 receptor alpha (IL-15Ra) polypeptide, or a combination of both an IL-15 polypeptide and an IL-15Ra polypeptide, e.g., hetIL-15; (vii) an interleukin-12 (IL-12) polypeptide; or (viii) an mTOR inhibitor, optionally wherein the mTOR inhibitor is RAD001 or rapamycin.

In some embodiments, provided herein is a cell comprising: (a) a first CAR comprising a first antigen-binding domain that binds to a first antigen, a first transmembrane domain, and a first intracellular signaling domain (e.g., a first primary signaling domain and/or a first costimulatory signaling domain), optionally wherein the first CAR further comprises a first leader sequence and/or a first hinge region; and (b) a second CAR comprising a second antigen-binding domain that binds to a second antigen, a second transmembrane domain, and a second intracellular signaling domain (e.g., a second primary signaling domain and/or a second costimulatory signaling domain), optionally wherein the second CAR further comprises a second leader sequence and/or a second hinge region, wherein: (i) the first leader sequence and the second leader sequence are encoded by different nucleic acid sequences (e.g., differ by at least 5%, 10%, 20%, 30%, 40%, 50%, 60%, 70%, 80%, 85%, 90%, 95%, or 100%), optionally wherein the first and second leader sequences comprise the same amino acid sequence; (ii) the first hinge region and the second hinge region are encoded by different nucleic acid sequences (e.g., differ by at least 5%, 10%, 20%, 30%, 40%, 50%, 60%, 70%, 80%, 85%, 90%, 95%, or 100%), optionally wherein the first and second hinge regions comprise the same amino acid sequence; (iii) the first transmembrane domain and the second transmembrane domain are encoded by different nucleic acid sequences (e.g., differ by at least 5%, 10%, 20%, 30%, 40%, 50%, 60%, 70%, 80%, 85%, 90%, 95%, or 100%), optionally wherein the first and second transmembrane domains comprise the same amino acid sequence; and/or (iv) the first intracellular signaling domain and the second intracellular signaling domain are encoded by different nucleic acid sequences (e.g., differ by at least 5%, 10%, 20%, 30%, 40%, 50%, 60%, 70%, 80%, 85%, 90%, 95%, or 100%), optionally wherein the first primary signaling domain and the second primary signaling domain are encoded by different nucleic acid sequences (e.g., differ by at least 5%, 10%, 20%, 30%, 40%, 50%, 60%, 70%, 80%, 85%, 90%, 95%, or 100%), and/or the first costimulatory signaling domain and the second costimulatory signaling domain are encoded by different nucleic acid sequences (e.g., differ by at least 5%, 10%, 20%, 30%, 40%, 50%, 60%, 70%, 80%, 85%, 90%, 95%, or 100%).

In some embodiments, provided herein is a nucleic acid molecule comprising: (a) a first nucleic acid sequence encoding a first CAR, wherein the first CAR comprises a first antigen-binding domain that binds to a first antigen, a first transmembrane domain, and a first intracellular signaling domain (e.g., a first primary signaling domain and/or a first costimulatory signaling domain), optionally wherein the first CAR further comprises a first leader sequence and/or a first hinge region; and (b) a second nucleic acid sequence encoding a second CAR, wherein the second CAR comprises a second antigen-binding domain that binds to a second antigen, a second transmembrane domain, and a second intracellular signaling domain (e.g., a second primary signaling domain and/or a second costimulatory signaling domain), optionally wherein the second CAR further comprises a second leader sequence and/or a second hinge region, wherein: (i) the first leader sequence and the second leader sequence are encoded by different nucleic acid sequences (e.g., differ by at least 5%, 10%, 20%, 30%, 40%, 50%, 60%, 70%, 80%, 85%, 90%, 95%, or 100%), optionally wherein the first and second leader sequences comprise the same amino acid sequence; (ii) the first hinge region and the second hinge region are encoded by different nucleic acid sequences (e.g., differ by at least 5%, 10%, 20%, 30%, 40%, 50%, 60%, 70%, 80%, 85%, 90%, 95%, or 100%), optionally wherein the first and second hinge regions comprise the same amino acid sequence; (iii) the first transmembrane domain and the second transmembrane domain are encoded by different nucleic acid sequences (e.g., differ by at least 5%, 10%, 20%, 30%, 40%, 50%, 60%, 70%, 80%, 85%, 90%, 95%, or 100%), optionally wherein the first and second transmembrane domains comprise the same amino acid sequence; and/or (iv) the first intracellular signaling domain and the second intracellular signaling domain are encoded by different nucleic acid sequences (e.g., differ by at least 5%, 10%, 20%, 30%, 40%, 50%, 60%, 70%, 80%, 85%, 90%, 95%, or 100%), optionally wherein the first intracellular signaling domain and the second intracellular signaling domain comprise the same amino acid sequence.

In some embodiments, the first and second leader sequences comprise the same amino acid sequence. Without wishing to be bound by theory, such a nucleic acid molecule exhibits less recombination than an otherwise similar nucleic acid molecule in which the first leader sequence and the second leader sequence are encoded by the same nucleic acid sequence.

In some embodiments, the first and second hinge regions comprise the same amino acid sequence. Without wishing to be bound by theory, such a nucleic acid molecule exhibits less recombination than an otherwise similar nucleic acid molecule in which the first hinge region and the second hinge region are encoded by the same nucleic acid sequence.

In some embodiments, the first and second transmembrane domains comprise the same amino acid sequence. Without wishing to be bound by theory, such a nucleic acid molecule exhibits less recombination than an otherwise similar nucleic acid molecule in which the first transmembrane domain and the second transmembrane domain are encoded by the same nucleic acid sequence.

In some embodiments, the first intracellular signaling domain and the second intracellular signaling domain comprise the same amino acid sequence. Without wishing to be bound by theory, such a nucleic acid molecule exhibits less recombination than an otherwise similar nucleic acid molecule in which the first intracellular signaling domain and the second intracellular signaling domain are encoded by the same nucleic acid sequence.

In some embodiments, the first primary signaling domain and the second primary signaling domain comprise the same amino acid sequence. Without wishing to be bound by theory, such a nucleic acid molecule exhibits less recombination than an otherwise similar nucleic acid molecule in which the first primary signaling domain and the second primary signaling domain are encoded by the same nucleic acid sequence.

In some embodiments, the first primary signaling domain and the second primary signaling domain comprise different amino acid sequences.

In some embodiments, the first costimulatory signaling domain and the second costimulatory signaling domain comprise the same amino acid sequence. Without wishing to be bound by theory, such a nucleic acid molecule exhibits less recombination than an otherwise similar nucleic acid molecule in which the first costimulatory signaling domain and the second costimulatory signaling domain are encoded by the same nucleic acid sequence.

In some embodiments, the first costimulatory signaling domain and the second costimulatory signaling domain comprise different amino acid sequences (e.g., the first and second costimulatory signaling domains comprise a 4-1BB costimulatory domain sequence and a CD28 costimulatory domain sequence, respectively; or comprise a CD28 costimulatory domain sequence and a 4-1BB costimulatory domain sequence, respectively).

In some embodiments, the first leader sequence and the second leader sequence comprise the amino acid sequence of SEQ ID NO: 1, or an amino acid sequence having at least about 85%, 90%, 95%, or 99% sequence identity thereto. In some embodiments, the first leader sequence and the second leader sequence are encoded by nucleic acid sequences comprising SEQ ID NOs: 199 and 210, respectively (or a nucleic acid sequence having at least about 85%, 90%, 95%, or 99% sequence identity thereto), or SEQ ID NOs: 210 and 199, respectively (or a nucleic acid sequence having at least about 85%, 90%, 95%, or 99% sequence identity thereto).

In some embodiments, the first hinge region and the second hinge region comprise the amino acid sequence of SEQ ID NO: 2, or an amino acid sequence having at least about 85%, 90%, 95%, or 99% sequence identity thereto. In some embodiments, the first hinge region and the second hinge region are encoded by nucleic acid sequences comprising SEQ ID NOs: 337 and 13, respectively (or a nucleic acid sequence having at least about 85%, 90%, 95%, or 99% sequence identity thereto); or SEQ ID NOs: 13 and 337, respectively (or a nucleic acid sequence having at least about 85%, 90%, 95%, or 99% sequence identity thereto).

In some embodiments, the first transmembrane domain and the second transmembrane domain comprise the amino acid sequence of SEQ ID NO: 6, or an amino acid sequence having at least about 85%, 90%, 95%, or 99% sequence identity thereto. In some embodiments, the first transmembrane domain and the second transmembrane domain are encoded by nucleic acid sequences comprising SEQ ID NOs: 338 and 17, respectively (or a nucleic acid sequence having at least about 85%, 90%, 95%, or 99% sequence identity thereto); or SEQ ID NOs: 17 and 338, respectively (or a nucleic acid sequence having at least about 85%, 90%, 95%, or 99% sequence identity thereto).

In some embodiments, the first costimulatory signaling domain and the second costimulatory signaling domain comprise the amino acid sequence of SEQ ID NO: 7, or an amino acid sequence having at least about 85%, 90%, 95%, or 99% sequence identity thereto. In some embodiments, the first costimulatory signaling domain and the second costimulatory signaling domain are encoded by nucleic acid sequences comprising SEQ ID NOs: 204 and 18, respectively (or a nucleic acid sequence having at least about 85%, 90%, 95%, or 99% sequence identity thereto); or SEQ ID NOs: 18 and 204, respectively (or a nucleic acid sequence having at least about 85%, 90%, 95%, or 99% sequence identity thereto).

In some embodiments, the first primary signaling domain and the second primary signaling domain comprise the amino acid sequence of SEQ ID NO: 10, or an amino acid sequence having at least about 85%, 90%, 95%, or 99% sequence identity thereto. In some embodiments, the first primary signaling domain and the second primary signaling domain are encoded by nucleic acid sequences comprising SEQ ID NOs: 205 and 21, respectively (or a nucleic acid sequence having at least about 85%, 90%, 95%, or 99% sequence identity thereto); or SEQ ID NOs: 21 and 205, respectively (or a nucleic acid sequence having at least about 85%, 90%, 95%, or 99% sequence identity thereto).

In some embodiments, the first and second antigens are different. In some embodiments, the first or second antigen is chosen from: BCMA, CD19, CD5, CD10, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, FcRn5, FcRn2, CS-1, CXCR4, 5, 7, IL-7/3R, IL7/4/3R, or IL4R, optionally wherein the B cell antigen is chosen from CD19, CD20, CD22, FcRn5, FcRn2, CS-1, CD138, CD123, CD33, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In some embodiments, the first or second antigen-binding domain comprises a CDR, VH, VL, or scFv disclosed herein, or an amino acid sequence having at least about 85%, 90%, 95%, or 99% sequence identity thereto.

In some embodiments, provided herein is a CAR comprising a first VH (VH1), a first VL (VL1), a second VH (VH2), a second VL (VL2), a transmembrane domain, and an intracellular signaling domain, wherein the VH1 ad VL1 bind to a first antigen and the VH2 and VL2 bind to a second antigen. In some embodiments, the VH1, VL1, VH2, and VL2 are arranged in the following configuration from the N-terminus to the C-terminus: VH1—optionally linker 1 (“L1”)-VH2—optionally linker 2 (“L2”)-VL2—optionally linker 3 (“L3”)-VL1. In some embodiments, the VH1, VL1, VH2, and VL2 are arranged in the following configuration from the N-terminus to the C-terminus: VH1—optionally L1-VL2—optionally L2-VH2—optionally L3-VL1. In some embodiments, the VH1, VL1, VH2, and VL2 are arranged in the following configuration from the N-terminus to the C-terminus: VL1—optionally L1-VH2—optionally L2-VL2—optionally L3-VH1. In some embodiments, the VH1, VL1, VH2, and VL2 are arranged in the following configuration from the N-terminus to the C-terminus: VL1—optionally L1-VL2—optionally L2-VH2—optionally L3-VH1. In some embodiments, the VH1, VL1, VH2, and VL2 are arranged in the following configuration from the N-terminus to the C-terminus: VH2—optionally L1-VH1—optionally L2-VL1—optionally L3-VL2. In some embodiments, the VH1, VL1, VH2, and VL2 are arranged in the following configuration from the N-terminus to the C-terminus: VH2—optionally L1-VL1—optionally L2-VH1—optionally L3-VL2. In some embodiments, the VH1, VL1, VH2, and VL2 are arranged in the following configuration from the N-terminus to the C-terminus: VL2—optionally L1-VH1—optionally L2-VL1—optionally L3-VH2. In some embodiments, the VH1, VL1, VH2, and VL2 are arranged in the following configuration from the N-terminus to the C-terminus: VL2—optionally L1-VL1—optionally L2-VH1—optionally L3-VH2. In some embodiments, the VH1, VL1, VH2, and VL2 are arranged in the following configuration from the N-terminus to the C-terminus: VH1—linker 1 (“L1”)-VH2—linker 2 (“L2”)-VL2-linker 3 (“L3”)-VL1. In some embodiments, the VH1, VL1, VH2, and VL2 are arranged in the following configuration from the N-terminus to the C-terminus: VH1—L1-VL2-L2-VH2—L3-VL1. In some embodiments, the VH1, VL1, VH2, and VL2 are arranged in the following configuration from the N-terminus to the C-terminus: VL1-L1-VH2—L2-VL2-L3-VH1. In some embodiments, the VH1, VL1, VH2, and VL2 are arranged in the following configuration from the N-terminus to the C-terminus: VL1-L1-VL2-L2-VH2—L3-VH1. In some embodiments, the VH1, VL1, VH2, and VL2 are arranged in the following configuration from the N-terminus to the C-terminus: VH2—L1-VH1—L2-VL1-L3-VL2. In some embodiments, the VH1, VL1, VH2, and VL2 are arranged in the following configuration from the N-terminus to the C-terminus: VH2—L1-VL1-L2-VH1—L3-VL2. In some embodiments, the VH1, VL1, VH2, and VL2 are arranged in the following configuration from the N-terminus to the C-terminus: VL2-L1-VH1—L2-VL1-L3-VH2. In some embodiments, the VH1, VL1, VH2, and VL2 are arranged in the following configuration from the N-terminus to the C-terminus: VL2-L1-VL1-L2-VH1—L3—VH2. In some embodiments, the L1 or L3 comprises the amino acid sequence of SEQ ID NO: 5, or an amino acid sequence having at least about 85%, 90%, 95%, or 99% sequence identity thereto. In some embodiments, the L2 comprises the amino acid sequence of SEQ ID NO: 63, or an amino acid sequence having at least about 85%, 90%, 95%, or 99% sequence identity thereto. In some embodiments, the CAR comprises the following configuration from the N-terminus to the C-terminus: (i) VH1—optionally linker 1 (“L1”)-VH2—optionally linker 2 (“L2”)-VL2—optionally linker 3 (“L3”)-VL1—optionally a hinge region—transmembrane domain—intracellular signaling domain; (ii) VH1—optionally L1-VL2—optionally L2-VH2—optionally L3-VL1—optionally a hinge region-transmembrane domain—intracellular signaling domain; (iii) VL1—optionally L1-VH2—optionally L2-VL2—optionally L3-VH1—optionally a hinge region—transmembrane domain—intracellular signaling domain; (iv) VL1—optionally L1-VL2—optionally L2-VH2—optionally L3-VH1—optionally a hinge region—transmembrane domain—intracellular signaling domain; (v) VH2—optionally L1-VH1—optionally L2-VL1—optionally L3-VL2—optionally a hinge region—transmembrane domain—intracellular signaling domain; (vi) VH2—optionally L1-VL1—optionally L2-VH1—optionally L3-VL2—optionally a hinge region—transmembrane domain—intracellular signaling domain; (vii) VL2—optionally L1-VH1—optionally L2-VL1—optionally L3-VH2—optionally a hinge region—transmembrane domain—intracellular signaling domain; or (viii) VL2—optionally L1-VL1—optionally L2-VH1—optionally L3-VH2—optionally a hinge region—transmembrane domain—intracellular signaling domain. In some embodiments, the first and second antigens are different. In some embodiments, the first or second antigen is chosen from: BCMA, CD19, CD5, CD10, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, FcRn5, FcRn2, CS-1, CXCR4, 5, 7, IL-7/3R, IL7/4/3R, or IL4R, optionally wherein the B cell antigen is chosen from CD19, CD20, CD22, FcRn5, FcRn2, CS-1, CD138, CD123, CD33, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In some embodiments, the VH1, VL1, VH2, or VL2 comprises a CDR, VH, or VL sequence disclosed herein, or an amino acid sequence having at least about 85%, 90%, 95%, or 99% sequence identity thereto. In some embodiments, the hinge region, transmembrane domain, or intracellular signaling domain (e.g., a primary signaling domain and/or a costimulatory signaling domain) comprises a hinge region sequence, transmembrane domain sequence, or intracellular signaling domain sequence (e.g., a primary signaling domain sequence and/or a costimulatory signaling domain sequence) disclosed herein, or an amino acid sequence having at least about 85%, 90%, 95%, or 99% sequence identity thereto.

In some embodiments, provided herein is a nucleic acid molecule encoding a diabody CAR disclosed herein. In some embodiments, provided herein is a vector comprising a nucleic acid molecule encoding a diabody CAR disclosed herein. In some embodiments, provided herein is a cell comprising a CAR disclosed herein, a nucleic acid molecule encoding a diabody CAR disclosed herein, or a vector comprising a nucleic acid molecule encoding a diabody CAR disclosed herein. In some embodiments, provided herein is a pharmaceutical composition comprising a cell comprising a diabody CAR disclosed herein and a pharmaceutically acceptable carrier. In some embodiments, disclosed herein is a method of making a cell comprising a diabody CAR disclosed herein. In some embodiments, disclosed herein is a method of treating a subject, e.g., a subject having cancer, using a cell comprising a diabody CAR disclosed herein.

In some embodiments, the present disclosure pertains to methods of making immune effector cells (for example, T cells or NK cells) engineered to express a CAR, and compositions generated using such methods. The methods disclosed herein (e.g., the ARM process or the cytokine process disclosed herein) can be used to make cells expressing dual CARs or diabody CARs disclosed herein. Also disclosed are methods of using such compositions for treating a disease, for example, cancer, in a subject.

In some embodiments, this invention features a method of making a population of cells (for example, T cells) that express a chimeric antigen receptor (CAR), the method comprising: (i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a nucleic acid molecule (for example, a DNA or RNA molecule) encoding the CAR, thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, 24, or 25 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 hours after the beginning of step (ii), for example, no later than 22, 23, 24, 25, 26, 27, 28, 29, or 30 hours after the beginning of step (ii); or (c) the population of cells from step (iii) are not expanded, or expanded by no more than 5, 6, 7, 8, 9, 10, 11, 12, 13, 14, 15, 16, 17, 18, 19, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) with a viral vector comprising a nucleic acid molecule encoding the CAR. In some embodiments, step (ii) is performed together with step (i). In some embodiments, step (ii) is performed no later than 20 hours after the beginning of step (i). In some embodiments, step (ii) is performed no later than 12, 13, 14, 15, 16, 17, or 18 hours after the beginning of step (i). In some embodiments, step (ii) is performed no later than 18 hours after the beginning of step (i). In some embodiments, step (iii) is performed no later than 26 hours after the beginning of step (i). In some embodiments, step (iii) is performed no later than 22, 23, 24, or 25 hours after the beginning of step (i). In some embodiments, step (iii) is performed no later than 24 hours after the beginning of step (i). In some embodiments, step (iii) is performed no later than 30 hours after the beginning of step (ii). In some embodiments, step (iii) is performed no later than 22, 23, 24, 25, 26, 27, 28, 29, or 30 hours after the beginning of step (ii). In some embodiments, the nucleic acid molecule encoding the CAR is a nucleic acid molecule disclosed herein. In some embodiments, the nucleic acid molecule comprises a first nucleic acid sequence encoding a first CAR and a second nucleic acid sequence encoding a second CAR. In some embodiments, the first and second nucleic acid sequences are disposed on a single nucleic acid molecule, e.g., wherein the first nucleic acid sequence and the second nucleic acid sequence are separated by a third nucleic acid sequence encoding a self-cleavage site (e.g., a P2A site, a T2A site, an E2A site, or an F2A site). In some embodiments, the first and second nucleic acid sequences are disposed on separate nucleic acid molecules. In some embodiments, the nucleic acid molecule comprises a nucleic acid sequence encoding a CAR, wherein the CAR comprises a first VH (VH1), a first VL (VL1), a second VH (VH2), a second VL (VL2), a transmembrane domain, and an intracellular signaling domain, wherein the VH1 ad VL1 bind to a first antigen and the VH2 and VL2 bind to a second antigen, wherein the VH1, VL1, VH2, and VL2 are arranged in the following configuration from the N-terminus to the C-terminus: VH1—optionally linker 1 (“L1”)-VH2—optionally linker 2 (“L2”)-VL2—optionally linker 3 (“L3”)-VL1, VH1—optionally L1-VL2—optionally L2-VH2—optionally L3-VL1, VL1—optionally L1-VH2—optionally L2-VL2—optionally L3-VH1, VL1—optionally L1-VL2—optionally L2-VH2—optionally L3-VH1, VH2—optionally L1-VH1—optionally L2-VL1—optionally L3-VL2, VH2—optionally L1-VL1—optionally L2-VH1—optionally L3-VL2, VL2—optionally L1-VH1—optionally L2-VL1—optionally L3-VH2; or VL2—optionally L1-VL1—optionally L2-VH1—optionally L3-VH2.

In some embodiments, the population of cells from step (iii) are not expanded. In some embodiments, the population of cells from step (iii) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (i). In some embodiments, the population of cells from step (iii) are expanded by no more than 10%, for example, as assessed by the number of living cells, compared to the population of cells at the beginning of step (i).

In some embodiments, the nucleic acid molecule comprises a first nucleic acid sequence encoding a first CAR and a second nucleic acid sequence encoding a second CAR, wherein the first and second nucleic acid sequences are disposed on separate nucleic acid molecules.

In some embodiments, the first and second nucleic acid molecules are on separate viral vectors, and wherein step (ii) comprises transducing the population of cells (for example, T cells) with a first viral vector comprising the nucleic acid molecule encoding the first CAR and a second viral vector comprising the second nucleic acid molecule encoding the second CAR.

In some embodiments, the first CAR comprises an anti-BCMA binding domain (e.g., an anti-BCMA CAR) and the second CAR comprises an anti-CD19 binding domain (e.g., an anti-CD19 CAR).

In some embodiments, in step (ii), the population of cells is contacted with the first viral vector at a multiplicity of infection (MOI) that is higher than, equal to, or less than an MOI at which the population of cells is contacted with the second viral vector. In some embodiments, in step (ii), the population of cells is contacted with the first viral vector at a multiplicity of infection (MOI) that is higher than an MOI at which the population of cells is contacted with the second viral vector.

In some embodiments, in step (ii), the population of cells is contacted with the first viral vector at a first MOI and with the second viral vector at a second MOI, such that a resultant population of cells comprises a first population of cells that comprise the anti-BCMA CAR but not the anti-CD19 CAR, a second population of cells that comprise the anti-CD19 CAR but not the anti-BCMA CAR, and a third population of cells that comprise both the anti-BCMA CAR and the anti-CD19 CAR, wherein:
(a) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10;
(b) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined, e.g., as determined by a method described in Example 10;
(c) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the resultant population, e.g., as determined by a method described in Example 10;
(d) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10; or
(e) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second population, e.g., as determined by a method described in Example 10. In some embodiments, in step (ii), the population of cells is contacted with the second viral vector at an MOI (e.g., an MOI that is sufficiently lower than an MOI at which the population of cells is contacted with the first viral vector, such that in a resultant population of cells:
(a) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10;
(b) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined, e.g., as determined by a method described in Example 10;
(c) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the resultant population, e.g., as determined by a method described in Example 10;
(d) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10; or
(e) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second population, e.g., as determined by a method described in Example 10.

In some embodiments, in step (ii), the population of cells is contacted with the first viral vector at a first MOI, and the population of cells is contacted with the second viral vector at a second MOI, such that a resultant population of cells comprises:
(a) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10;
(b) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined, e.g., as determined by a method described in Example 10;
(c) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the resultant population, e.g., as determined by a method described in Example 10;
(d) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10; or
(e) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second population, e.g., as determined by a method described in Example 10.

In some embodiments, in step (ii), the population of cells is contacted with:
(a) the first viral vector at an MOI of about 1 to about 10 (e.g., about 2 to about 9, about 3 to about 8, about 4 to about 7, about 5 to about 6, about 1 to about 8, about 1 to about 6, about 1 to about 4, about 8 to about 10, about 6 to about 10, about 4 to about 10, about 1 to about 3, about 2 to about 4, about 3 to about 5, about 4 to about 6, about 5 to about 7, about 6 to about 8, about 7 to about to about 9, about 8 to about 10, about 2.5 to about 5, about 1, about 2, about 3, about 4, about 5, about 6, about 7, about 8, about 9, or about 10);
(b) the second viral vector at an MOI of about 0.1 to about 5 (e.g., about 0.2 to about 4, about 0.3 to about 3, about 0.4 to about 2, about 0.5 to about 1, about 0.6 to about 0.9, about 0.7 to about 0.8, about 0.1 to about 4, about 0.1 to about 3, about 0.1 to about 2, about 0.1 to about 1, about 0.1 to about 0.5, about 4 to about 5, about 3 to about 5, about 2 to about 5, about 1 to about 5, about 0.5 to about 5, about 0.2 to about 5, about 0.1 to about 0.5, about 0.2 to about 1, about 0.5 to about 2, about 1 to about 3, about 2 to about 4, about 3 to about 5, about 0.5 to about 1, about 0.1, about 0.2, about 0.3, about 0.4, about 0.5, about 0.6, about 0.7, about 0.8, about 0.9, about 1, about 2, about 3, about 4, or about 5);
(c) the first viral vector at an MOI that is at least about 10% (e.g., at least about 20%, 30%, 40%, 50%, 60%, 70%, 80%, or 90%) or at least about 1 fold (e.g., at least about 2, 3, 4, 5, 6, 7, 8, 9, 10, 15, 20, 25, 30, 35, 40, 50, 60, 70, 80, 90, or 100 fold, e.g., about 2 to about 50 fold, about 3 to 20 fold, about 5 to about 15 fold, or about 8 to about 10 fold) higher than an MOI at which the population of cells is contacted with the second viral vector; and/or
(d) the second viral vector at an MOI that is no more than 1/X, wherein X is 2, 3, 4, 5, 6, 7, 8, 9, 10, 12, 15, 20, 30, 40, 50, 60, 70. 80, 90, or 100, of an MOI at which the population of cells is contacted with the first viral vector.

In some embodiments, the population of cells is contacted with the first viral vector at an MOI of about 2.5 to about 5. In some embodiments, the population of cells is contacted with the second viral vector at an MOI of about 0.5 to about 1.0. In some embodiments, the first viral vector at an MOI that is about 8 to about 10 fold higher than an MOI at which the population of cells is contacted with the second viral vector. In some embodiments, the second viral vector at an MOI that is no more than 1/X, wherein X is 6, 8, 10, or 12, of an MOI at which the population of cells is contacted with the first viral vector.

In some embodiments, in step (ii), the population of cells is contacted with:
(a) the first viral vector at an MOI of between about 4 and about 5 (e.g., about 4.75); and/or
(b) the second viral vector at an MOI between about 0.2 and about 1 (e.g., about 0.5).

In some embodiments, in step (ii), the population of cells comprises about 1×10⁸ to about 5×10⁹ (e.g., about 2×10⁸ to about 2×10⁹ or about 4×10⁸ to about 1×10⁹ total viable cells. In some embodiments, the cells are suspended in a culture at a concentration of about 1×10⁶ to about 1×10⁷ (e.g., about 2×10⁶ to about 5×10⁶ or about 3×10⁶ to about 4×10⁶) viable cells/mL.

In some embodiments, the agent that stimulates a CD3/TCR complex is an agent that stimulates CD3. In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex does not comprise a bead. In some embodiments, the agent that stimulates a costimulatory molecule does not comprise a bead. In some embodiments, the agent that stimulates a CD3/TCR complex comprises an anti-CD3 antibody. In some embodiments, the agent that stimulates a costimulatory molecule comprises an anti-CD28 antibody. In some embodiments, the agent that stimulates a CD3/TCR complex comprises an anti-CD3 antibody covalently attached to a colloidal polymeric nanomatrix. In some embodiments, the agent that stimulates a costimulatory molecule comprises an anti-CD28 antibody covalently attached to a colloidal polymeric nanomatrix. In some embodiments, the agent that stimulates a CD3/TCR complex and the agent that stimulates a costimulatory molecule comprise T Cell TransAct™.

In some embodiments, the agent that stimulates a CD3/TCR complex does not comprise hydrogel. In some embodiments, the agent that stimulates a costimulatory molecule does not comprise hydrogel. In some embodiments, the agent that stimulates a CD3/TCR complex does not comprise alginate. In some embodiments, the agent that stimulates a costimulatory molecule does not comprise alginate.

In some embodiments, the agent that stimulates a CD3/TCR complex comprises hydrogel. In some embodiments, the agent that stimulates a costimulatory molecule comprises hydrogel. In some embodiments, the agent that stimulates a CD3/TCR complex comprises alginate. In some embodiments, the agent that stimulates a costimulatory molecule comprises alginate. In some embodiments, the agent that stimulates a CD3/TCR complex or the agent that stimulates a costimulatory molecule comprises MagCloudz™ from Quad Technologies.

In some embodiments, step (i) increases the percentage of CAR-expressing cells in the population of cells from step (iii), for example, the population of cells from step (iii) shows a higher percentage of CAR-expressing cells (for example, at least 10, 20, 30, 40, 50, or 60% higher), compared with cells made by an otherwise similar method without step (i).

In some embodiments, the percentage of naïve cells, for example, naïve T cells, for example, CD45RA+CD45RO− CCR7+ T cells, in the population of cells from step (iii) is the same as the percentage of naïve cells, for example, naïve T cells, for example, CD45RA+CD45RO− CCR7+ cells, in the population of cells at the beginning of step (i). In some embodiments, the percentage of naïve cells, for example, naïve T cells, for example, CD45RA+CD45RO− CCR7+ T cells, in the population of cells from step (iii) differs by no more than 3, 4, 5, 6, 7, 8, 9, 10, 11, or 12% from the percentage of naïve cells, for example, naïve T cells, for example, CD45RA+CD45RO− CCR7+ cells, in the population of cells at the beginning of step (i). In some embodiments, the percentage of naïve cells, for example, naïve T cells, for example, CD45RA+CD45RO− CCR7+ T cells, in the population of cells from step (iii) differs by no more than 5 or 10% from the percentage of naïve cells, for example, naïve T cells, for example, CD45RA+CD45RO− CCR7+ cells, in the population of cells at the beginning of step (i).

In some embodiments, the population of cells from step (iii) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of central memory cells, for example, central memory T cells, for example, CD95+ central memory T cells, in the population of cells from step (iii) is the same as the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (iii) differs by no more than 3, 4, 5, 6, 7, 8, 9, 10, 11, or 12% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (iii) differs by no more than 5 or 10% from the percentage of central memory cells, for example, central memory T cells, for example, CD95+ central memory T cells, in the population of cells at the beginning of step (i).

In some embodiments, the population of cells from step (iii) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the percentage of stem memory T cells, for example, CD45RA+CD95+IL-2 receptor β+CCR7+CD62L+ T cells, in the population of cells from step (iii) is increased, as compared to the percentage of stem memory T cells, for example, CD45RA+CD95+IL-2 receptor β+CCR7+CD62L+ T cells, in the population of cells at the beginning of step (i). In some embodiments, the percentage of CAR-expressing stem memory T cells, for example, CAR-expressing CD45RA+CD95+IL-2 receptor β+CCR7+CD62L+ T cells, in the population of cells from step (iii) is increased, as compared to the percentage of CAR-expressing stem memory T cells, for example, CAR-expressing CD45RA+CD95+IL-2 receptor β+CCR7+CD62L+ T cells, in the population of cells at the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method in which step (iii) is performed more than 26 hours after the beginning of step (i), for example, more than 5, 6, 7, 8, 9, 10, 11, or 12 days after the beginning of step (i). In some embodiments, the percentage of stem memory T cells, for example, CD45RA+CD95+IL-2 receptor β+CCR7+CD62L+ T cells, in the population of cells from step (iii) is higher than the percentage of stem memory T cells, for example, CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the percentage of CAR-expressing stem memory T cells, for example, CAR-expressing CD45RA+CD95+IL-2 receptor β+CCR7+CD62L+ T cells, in the population of cells from step (iii) is higher than the percentage of CAR-expressing stem memory T cells, for example, CAR-expressing CD45RA+CD95+IL-2 receptor β+CCR7+CD62L+ T cells, in cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the median GeneSetScore (Up TEM vs. Down TSCM) of the population of cells from step (iii) is about the same as or differs by no more than (for example, increased by no more than) about 25, 50, 75, 100, or 125% from the median GeneSetScore (Up TEM vs. Down TSCM) of the population of cells at the beginning of step (i). In some embodiments, the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up TEM vs. Down TSCM) of the population of cells from step (iii) is lower (for example, at least about 100, 150, 200, 250, or 300% lower) than the median GeneSetScore (Up TEM vs. Down TSCM) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up Treg vs. Down Teff) of the population of cells from step (iii) is about the same as or differs by no more than (for example, increased by no more than) about 25, 50, 100, 150, or 200% from the median GeneSetScore (Up Treg vs. Down Teff) of the population of cells at the beginning of step (i). In some embodiments, the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up Treg vs. Down Teff) of the population of cells from step (iii) is lower (for example, at least about 50, 100, 125, 150, or 175% lower) than the median GeneSetScore (Up Treg vs. Down Teff) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Down stemness) of the population of cells from step (iii) is about the same as or differs by no more than (for example, increased by no more than) about 25, 50, 100, 150, 200, or 250% from the median GeneSetScore (Down stemness) of the population of cells at the beginning of step (i). In some embodiments, the median GeneSetScore (Down stemness) of the population of cells from step (iii) is lower (for example, at least about 50, 100, or 125% lower) than the median GeneSetScore (Down stemness) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Down stemness) of the population of cells from step (iii) is lower (for example, at least about 50, 100, or 125% lower) than the median GeneSetScore (Down stemness) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up hypoxia) of the population of cells from step (iii) is about the same as or differs by no more than (for example, increased by no more than) about 125, 150, 175, or 200% from the median GeneSetScore (Up hypoxia) of the population of cells at the beginning of step (i). In some embodiments, the median GeneSetScore (Up hypoxia) of the population of cells from step (iii) is lower (for example, at least about 40, 50, 60, 70, or 80% lower) than the median GeneSetScore (Up hypoxia) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up hypoxia) of the population of cells from step (iii) is lower (for example, at least about 40, 50, 60, 70, or 80% lower) than the median GeneSetScore (Up hypoxia) of cells made by an otherwise similar method which further comprises, after step (ii) and prior to step (iii), expanding the population of cells (for example, T cells) in vitro for more than 3 days, for example, for 5, 6, 7, 8 or 9 days. In some embodiments, the median GeneSetScore (Up autophagy) of the population of cells from step (iii) is about the same as or differs by no more than (for example, increased by no more than) about 180, 190, 200, or 210% from the median GeneSetScore (Up autophagy) of the population of cells at the beginning of step (i). In some embodiments, the median GeneSetScore (Up autophagy) of the population of cells from step (iii) is lower (for example, at least 20, 30, or 40% lower) than the median GeneSetScore (Up autophagy) of cells made by an otherwise similar method in which step (iii) is performed more than 26 hours after the beginning of step (i), for example, more than 5, 6, 7, 8, 9, 10, 11, or 12 days after the beginning of step (i). In some embodiments, the median GeneSetScore (Up autophagy) of the population of cells from step (iii) is lower (for example, at least 20, 30, or 40% lower) than the median GeneSetScore (Up autophagy) of cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being incubated with a cell expressing an antigen recognized by the CAR, secretes IL-2 at a higher level (for example, at least 2, 4, 6, 8, 10, 12, or 14-fold higher) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method in which step (iii) is performed more than 26 hours after the beginning of step (i), for example, more than 5, 6, 7, 8, 9, 10, 11, or 12 days after the beginning of step (i). In some embodiments, the population of cells from step (iii), after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii), after being administered in vivo, shows a stronger anti-tumor activity (for example, a stronger anti-tumor activity at a low dose, for example, a dose no more than 0.15×10⁶, 0.2×10⁶, 0.25×10⁶, or 0.3×10⁶ viable CAR-expressing cells) than cells made by an otherwise similar method in which step (iii) is performed more than 26 hours after the beginning of step (i), for example, more than 5, 6, 7, 8, 9, 10, 11, or 12 days after the beginning of step (i), or cells made by an otherwise similar method which further comprises, after step (ii) and prior to step (iii), expanding the population of cells (for example, T cells) in vitro for more than 3 days, for example, for 5, 6, 7, 8 or 9 days.

In some embodiments, the population of cells from step (iii) are not expanded, for example, as assessed by the number of living cells, compared to the population of cells at the beginning of step (i). In some embodiments, the population of cells from step (iii) decreases from the number of living cells in the population of cells at the beginning of step (i), for example, as assessed by the number of living cells. In some embodiments, the population of cells from step (iii) are expanded by no more than 5, 6, 7, 8, 9, 10, 11, 12, 13, 14, 15, 16, 17, 18, 19, 20, 25, 30, 35, or 40%, for example, no more than 10%, for example, as assessed by the number of living cells, compared to the population of cells at the beginning of step (i). In some embodiments, the population of cells from step (iii) are not expanded, or expanded by less than 0.5, 1, 1.5, or 2 hours, for example, less than 1 or 1.5 hours, compared to the population of cells at the beginning of step (i).

In some embodiments, steps (i) and (ii) are performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, steps (i) and (ii) are performed in cell media (for example, serum-free media) comprising IL-7, IL-21, or a combination thereof. In some embodiments, steps (i) and (ii) are performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, step (i) is performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, step (ii) is performed in cell media (for example, serum-free media) comprising IL-2, IL-15 (for example, hetIL-15 (IL15/sIL-15Ra)), IL-6 (for example, IL-6/sIL-6Ra), a LSD1 inhibitor, or a MALT1 inhibitor. In some embodiments, step (i) is performed in cell media (for example, serum-free media) comprising IL-7, IL-21, or a combination thereof. In some embodiments, step (ii) is performed in cell media (for example, serum-free media) comprising IL-7, IL-21, or a combination thereof. In some embodiments, step (i) is performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, step (ii) is performed in cell media (for example, serum-free media) comprising IL-2, IL-15 (for example, hetIL-15 (IL15/sIL-15Ra)), IL-21, IL-7, IL-6 (for example, IL-6/sIL-6Ra), a LSD1 inhibitor, a MALT1 inhibitor, or a combination thereof. In some embodiments, the cell media is a serum-free media comprising a serum replacement. In some embodiments, the serum replacement is CTS™ Immune Cell Serum Replacement (ICSR).

In some embodiments, the aforementioned methods further comprise prior to step (i): (iv) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the aforementioned methods further comprise prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

In some embodiments, the aforementioned methods further comprise prior to step (i): (iv) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the cells from step (iii) are cultured for about two to about four days, e.g., about three days (e.g., about 72 hours following harvesting) prior to measuring CAR expression level in the portion (for example, measuring the percentage of viable, CAR-expressing cells in the portion, for example, measuring the percentage of viable, anti-BCMA CAR-expressing cells in the portion). In some embodiments, the measuring of CAR expression occurs about 4 days (e.g., 96 hours) after step (ii). In some embodiments, the CAR expression level is measured by flow cytometry.

In some embodiments, this invention features a method of making a population of cells (for example, T cells) that express a chimeric antigen receptor (CAR), the method comprising: (1) contacting a population of cells (for example, T cells, for example, T cells isolated from a frozen leukapheresis product) with a cytokine chosen from IL-2, IL-7, IL-15, IL-21, IL-6, or a combination thereof, (2) contacting the population of cells (for example, T cells) with a nucleic acid molecule (for example, a DNA or RNA molecule) encoding the CAR, thereby providing a population of cells (for example, T cells) comprising the nucleic acid molecule, and (3) harvesting the population of cells (for example, T cells) for storage (for example, reformulating the population of cells in cryopreservation media) or administration, wherein: (a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, 24, or 25 hours after the beginning of step (1), for example, no later than 24 hours after the beginning of step (1), or (b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the nucleic acid molecule in step (2) is a DNA molecule. In some embodiments, the nucleic acid molecule in step (2) is an RNA molecule. In some embodiments, the nucleic acid molecule in step (2) is on a viral vector, for example, a viral vector chosen from a lentivirus vector, an adenoviral vector, or a retrovirus vector. In some embodiments, the nucleic acid molecule in step (2) is on a non-viral vector. In some embodiments, the nucleic acid molecule in step (2) is on a plasmid. In some embodiments, the nucleic acid molecule in step (2) is not on any vector. In some embodiments, step (2) comprises transducing the population of cells (for example, T cells) with a viral vector comprising a nucleic acid molecule encoding the CAR. In some embodiments, the nucleic acid molecule encoding the CAR is a nucleic acid molecule disclosed herein. In some embodiments, the nucleic acid molecule comprises a first nucleic acid sequence encoding a first CAR and a second nucleic acid sequence encoding a second CAR. In some embodiments, the first and second nucleic acid sequences are disposed on a single nucleic acid molecule, e.g., wherein the first nucleic acid sequence and the second nucleic acid sequence are separated by a third nucleic acid sequence encoding a self-cleavage site (e.g., a P2A site, a T2A site, an E2A site, or an F2A site). In some embodiments, the first and second nucleic acid sequences are disposed on separate nucleic acid molecules. In some embodiments, the nucleic acid molecule comprises a nucleic acid sequence encoding a CAR, wherein the CAR comprises a first VH (VH1), a first VL (VL1), a second VH (VH2), a second VL (VL2), a transmembrane domain, and an intracellular signaling domain, wherein the VH1 ad VL1 bind to a first antigen and the VH2 and VL2 bind to a second antigen, wherein the VH1, VL1, VH2, and VL2 are arranged in the following configuration from the N-terminus to the C-terminus: VH1—optionally linker 1 (“L1”)-VH2—optionally linker 2 (“L2”)-VL2—optionally linker 3 (“L3”)-VL1, VH1—optionally L1-VL2—optionally L2-VH2—optionally L3-VL1, VL1—optionally L1-VH2—optionally L2-VL2—optionally L3-VH1, VL1—optionally L1-VL2—optionally L2-VH2—optionally L3-VH1, VH2—optionally L1-VH1—optionally L2-VL1—optionally L3-VL2, VH2—optionally L1-VL1—optionally L2-VH1—optionally L3-VL2, VL2—optionally L1-VH1—optionally L2-VL1—optionally L3-VH2; or VL2—optionally L1-VL1—optionally L2-VH1—optionally L3-VH2.

In some embodiments, step (2) is performed together with step (1). In some embodiments, step (2) is performed no later than 5 hours after the beginning of step (1). In some embodiments, step (2) is performed no later than 1, 2, 3, 4, or 5 hours after the beginning of step (1). In some embodiments, step (3) is performed no later than 26 hours after the beginning of step (1). In some embodiments, step (3) is performed no later than 22, 23, 24, or 25 hours after the beginning of step (1). In some embodiments, step (3) is performed no later than 24 hours after the beginning of step (1).

In some embodiments, the population of cells from step (3) are not expanded, for example, as assessed by the number of living cells, compared to the population of cells at the beginning of step (1). In some embodiments, the population of cells from step (3) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (1). In some embodiments, the population of cells from step (3) are expanded by no more than 10%, for example, as assessed by the number of living cells, compared to the population of cells at the beginning of step (1).

In some embodiments, step (1) comprises contacting the population of cells (for example, T cells) with IL-2. In some embodiments, step (1) comprises contacting the population of cells (for example, T cells) with IL-7. In some embodiments, step (1) comprises contacting the population of cells (for example, T cells) with IL-15 (for example, hetIL-15 (IL15/sIL-15Ra)). In some embodiments, step (1) comprises contacting the population of cells (for example, T cells) with IL-21. In some embodiments, step (1) comprises contacting the population of cells (for example, T cells) with IL-6 (for example, IL-6/sIL-6Ra). In some embodiments, step (1) comprises contacting the population of cells (for example, T cells) with IL-2 and IL-7. In some embodiments, step (1) comprises contacting the population of cells (for example, T cells) with IL-2 and IL-15 (for example, hetIL-15 (IL15/sIL-15Ra)). In some embodiments, step (1) comprises contacting the population of cells (for example, T cells) with IL-2 and IL-21. In some embodiments, step (1) comprises contacting the population of cells (for example, T cells) with IL-2 and IL-6 (for example, IL-6/sIL-6Ra). In some embodiments, step (1) comprises contacting the population of cells (for example, T cells) with IL-7 and IL-15 (for example, hetIL-15 (IL15/sIL-15Ra)). In some embodiments, step (1) comprises contacting the population of cells (for example, T cells) with IL-7 and IL-21. In some embodiments, step (1) comprises contacting the population of cells (for example, T cells) with IL-7 and IL-6 (for example, IL-6/sIL-6Ra). In some embodiments, step (1) comprises contacting the population of cells (for example, T cells) with IL-15 (for example, hetIL-15 (IL15/sIL-15Ra)) and IL-21. In some embodiments, step (1) comprises contacting the population of cells (for example, T cells) with IL-15 (for example, hetIL-15 (IL15/sIL-15Ra)) and IL-6 (for example, IL-6/sIL-6Ra). In some embodiments, step (1) comprises contacting the population of cells (for example, T cells) with IL-21 and IL-6 (for example, IL-6/sIL-6Ra). In some embodiments, step (1) comprises contacting the population of cells (for example, T cells) with IL-7, IL-15 (for example, hetIL-15 (IL15/sIL-15Ra)), and IL-21.

In some embodiments, the population of cells from step (3) shows a higher percentage of naïve cells among CAR-expressing cells (for example, at least 10, 11, 12, 13, 14, 15, 16, 17, 18, 19, 20, 25, 30, 35, or 40% higher), compared with cells made by an otherwise similar method which further comprises contacting the population of cells with, for example, an anti-CD3 antibody.

In some embodiments, the percentage of naïve cells, for example, naïve T cells, for example, CD45RA+CD45RO− CCR7+ T cells, in the population of cells from step (3) is the same as the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) differs by no more than 3, 4, 5, 6, 7, 8, 9, 10, 11, or 12% from the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) differs by no more than 5 or 10% from the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) is increased as compared to the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) is increased by at least 10, 11, 12, 13, 14, 15, 16, 17, 18, 19, or 20%, as compared to the percentage of naïve cells, for example, naïve T cells, for example, CD45RA+CD45RO− CCR7+ cells, in the population of cells at the beginning of step (1). In some embodiments, the percentage of naïve cells, for example, naïve T cells, for example, CD45RA+CD45RO− CCR7+ T cells, in the population of cells from step (3) is increased by at least 10 or 20%, as compared to the percentage of naïve cells, for example, naïve T cells, for example, CD45RA+CD45RO− CCR7+ cells, in the population of cells at the beginning of step (1).

In some embodiments, the population of cells from step (3) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3) shows a higher percentage of naïve cells, for example, naïve T cells, for example, CD45RA+CD45RO− CCR7+ T cells (for example, at least 10, 11, 12, 13, 14, 15, 16, 17, 18, 19, 20, 25, 30, 35, or 40% higher), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the percentage of central memory cells, for example, central memory T cells, for example, CD95+ central memory T cells, in the population of cells from step (3) is the same as the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) differs by no more than 3, 4, 5, 6, 7, 8, 9, 10, 11, or 12% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) differs by no more than 5 or 10% from the percentage of central memory cells, for example, central memory T cells, for example, CD95+ central memory T cells, in the population of cells at the beginning of step (i). In some embodiments, the percentage of central memory cells, for example, central memory T cells, for example, CD95+ central memory T cells, in the population of cells from step (3) is decreased as compared to the percentage of central memory cells, for example, central memory T cells, for example, CD95+ central memory T cells, in the population of cells at the beginning of step (1). In some embodiments, the percentage of central memory cells, for example, central memory T cells, for example, CD95+ central memory T cells, in the population of cells from step (3) is decreased by at least 10 or 20%, as compared to the percentage of central memory cells, for example, central memory T cells, for example, CD95+ central memory T cells, in the population of cells at the beginning of step (1). In some embodiments, the percentage of central memory cells, for example, central memory T cells, for example, CD95+ central memory T cells, in the population of cells from step (3) is decreased by at least 10, 11, 12, 13, 14, 15, 16, 17, 18, 19, or 20%, as compared to the percentage of central memory cells, for example, central memory T cells, for example, CD95+ central memory T cells, in the population of cells at the beginning of step (1).

In some embodiments, the population of cells from step (3) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3) shows a lower percentage of central memory cells, for example, central memory T cells, for example, CD95+ central memory T cells (for example, at least 10, 11, 12, 13, 14, 15, 16, 17, 18, 19, 20, 25, 30, 35, or 40% lower), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3), after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method in which step (3) is performed more than 26 hours after the beginning of step (1), for example, more than 5, 6, 7, 8, 9, 10, 11, or 12 days after the beginning of step (1). In some embodiments, the population of cells from step (3), after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method which further comprises, after step (2) and prior to step (3), expanding the population of cells (for example, T cells) in vitro for more than 3 days, for example, for 5, 6, 7, 8 or 9 days.

In some embodiments, the population of cells from step (3) are not expanded, for example, as assessed by the number of living cells, compared to the population of cells at the beginning of step (1). In some embodiments, the population of cells from step (3) are expanded by no more than 5, 6, 7, 8, 9, 10, 11, 12, 13, 14, 15, 16, 17, 18, 19, 20, 25, 30, 35, or 40%, for example, as assessed by the number of living cells, compared to the population of cells at the beginning of step (1). In some embodiments, the population of cells from step (3) are expanded by no more than 10%, for example, as assessed by the number of living cells, compared to the population of cells at the beginning of step (1). In some embodiments, the number of living cells in the population of cells from step (3) decreases from the number of living cells in the population of cells at the beginning of step (1), for example, as assessed by the number of living cells.

In some embodiments, the population of cells from step (3) are not expanded compared to the population of cells at the beginning of step (1), for example, as assessed by the number of living cells. In some embodiments, the population of cells from step (3) are expanded by less than 0.5, 1, 1.5, or 2 hours, for example, less than 1 or 1.5 hours, compared to the population of cells at the beginning of step (1).

In some embodiments, the population of cells is not contacted in vitro with an agent that stimulates a CD3/TCR complex and/or an agent that stimulates a costimulatory molecule on the surface of the cells, or if contacted, the contacting step is less than 2 hours, for example, no more than 1 or 1.5 hours. In some embodiments, the agent that stimulates a CD3/TCR complex is an agent that stimulates CD3 (for example, an anti-CD3 antibody). In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule is an agent that stimulates CD28. In some embodiments, the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

In some embodiments, steps (1) and/or (2) are performed in cell media comprising no more than 5, 4, 3, 2, 1, or 0% serum. In some embodiments, steps (1) and/or (2) are performed in cell media comprising no more than 2% serum. In some embodiments, steps (1) and/or (2) are performed in cell media comprising about 2% serum. In some embodiments, steps (1) and/or (2) are performed in cell media comprising a LSD1 inhibitor or a MALT1 inhibitor. In some embodiments, step (1) is performed in cell media comprising no more than 5, 4, 3, 2, 1, or 0% serum. In some embodiments, step (1) is performed in cell media comprising no more than 2% serum. In some embodiments, step (1) is performed in cell media comprising about 2% serum. In some embodiments, step (2) is performed in cell media comprising no more than 5, 4, 3, 2, 1, or 0% serum. In some embodiments, step (2) is performed in cell media comprising no more than 2% serum. In some embodiments, step (2) is performed in cell media comprising about 2% serum. In some embodiments, step (1) is performed in cell media comprising a LSD1 inhibitor or a MALT1 inhibitor. In some embodiments, step (2) is performed in cell media comprising a LSD1 inhibitor or a MALT1 inhibitor.

In some embodiments, the aforementioned methods further comprise prior to step (i): (iv) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the aforementioned methods further comprise prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

In some embodiments, the aforementioned methods further comprise prior to step (i): (iv) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

In some embodiments, the aforementioned methods further comprise prior to step (i): (v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)). In some embodiments, step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v). In some embodiments, the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

In some embodiments, the population of cells at the beginning of step (i) or step (1) has been enriched for IL6R-expressing cells (for example, cells that are positive for IL6Rα and/or IL6Rβ). In some embodiments, the population of cells at the beginning of step (i) or step (1) comprises no less than 40, 45, 50, 55, 60, 65, or 70% of IL6R-expressing cells (for example, cells that are positive for IL6Rα and/or IL6Rβ).

In some embodiments, steps (i) and (ii) or steps (1) and (2) are performed in cell media comprising IL-15 (for example, hetIL-15 (IL15/sIL-15Ra)). In some embodiments, IL-15 increases the ability of the population of cells to expand, for example, 10, 15, 20, or 25 days later. In some embodiments, IL-15 increases the percentage of IL6Rβ-expressing cells in the population of cells.

In some embodiments of the aforementioned methods, the methods are performed in a closed system. In some embodiments, T cell separation, activation, transduction, incubation, and washing are all performed in a closed system. In some embodiments of the aforementioned methods, the methods are performed in separate devices. In some embodiments, T cell separation, activation and transduction, incubation, and washing are performed in separate devices.

In some embodiments of the aforementioned methods, the methods further comprise adding an adjuvant or a transduction enhancement reagent in the cell culture medium to enhance transduction efficiency. In some embodiments, the adjuvant or transduction enhancement reagent comprises a cationic polymer. In some embodiments, the adjuvant or transduction enhancement reagent is chosen from: LentiBOOST™ (Sirion Biotech), vectofusin-1, F108, hexadimethrine bromide (Polybrene), PEA, Pluronic F68, Pluronic F127, Synperonic or LentiTrans™. In some embodiments, the adjuvant is LentiBOOST™ (Sirion Biotech).

In some embodiments of the aforementioned methods, the transducing the population of cells (for example, T cells) with a viral vector comprises subjecting the population of cells and viral vector to a centrifugal force under conditions such that transduction efficiency is enhanced. In an embodiment, the cells are transduced by spinoculation.

In some embodiments of the aforementioned methods, cells (e.g., T cells) are activated and transduced in a cell culture flask comprising a gas-permeable membrane at the base that supports large media volumes without substantially compromising gas exchange. In some embodiments, cell growth is achieved by providing access, e.g., substantially uninterrupted access, to nutrients through convection.

In some embodiments of the aforementioned methods, the CAR comprises an antigen binding domain, a transmembrane domain, and an intracellular signaling domain.

In some embodiments, the antigen binding domain binds to an antigen chosen from: CD19, CD20, CD22, BCMA, mesothelin, EGFRvIII, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (for example, ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In some embodiments, the antigen binding domain comprises a CDR, VH, VL, scFv or a CAR sequence disclosed herein. In some embodiments, the antigen binding domain comprises a VH and a VL, wherein the VH and VL are connected by a linker, optionally wherein the linker comprises the amino acid sequence of SEQ ID NO: 63 or 104.

In some embodiments, the transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 and CD154. In some embodiments, the transmembrane domain comprises a transmembrane domain of CD8. In some embodiments, the transmembrane domain comprises the amino acid sequence of SEQ ID NO: 6, or an amino acid sequence having at least about 85%, 90%, 95%, or 99% sequence identity thereof. In some embodiments, the nucleic acid molecule comprises a nucleic acid sequence encoding the transmembrane domain, wherein the nucleic acid sequence comprises the nucleic acid sequence of SEQ ID NO: 17, or a nucleic acid sequence having at least about 85%, 90%, 95%, or 99% sequence identity thereof.

In some embodiments, the antigen binding domain is connected to the transmembrane domain by a hinge region. In some embodiments, the hinge region comprises the amino acid sequence of SEQ ID NO: 2, 3, or 4, or an amino acid sequence having at least about 85%, 90%, 95%, or 99% sequence identity thereof. In some embodiments, the nucleic acid molecule comprises a nucleic acid sequence encoding the hinge region, wherein the nucleic acid sequence comprises the nucleic acid sequence of SEQ ID NO: 13, 14, or 15, or a nucleic acid sequence having at least about 85%, 90%, 95%, or 99% sequence identity thereof.

In some embodiments, the intracellular signaling domain comprises a primary signaling domain. In some embodiments, the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεRI, DAP10, DAP12, or CD66d. In some embodiments, the primary signaling domain comprises a functional signaling domain derived from CD3 zeta. In some embodiments, the primary signaling domain comprises the amino acid sequence of SEQ ID NO: 9 or 10, or an amino acid sequence having at least about 85%, 90%, 95%, or 99% sequence identity thereof. In some embodiments, the nucleic acid molecule comprises a nucleic acid sequence encoding the primary signaling domain, wherein the nucleic acid sequence comprises the nucleic acid sequence of SEQ ID NO: 20 or 21, or a nucleic acid sequence having at least about 85%, 90%, 95%, or 99% sequence identity thereof.

In some embodiments, the intracellular signaling domain comprises a costimulatory signaling domain. In some embodiments, the costimulatory signaling domain comprises a functional signaling domain derived from a MHC class I molecule, a TNF receptor protein, an Immunoglobulin-like protein, a cytokine receptor, an integrin, a signaling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83. In some embodiments, the costimulatory signaling domain comprises a functional signaling domain derived from 4-1BB. In some embodiments, the costimulatory signaling domain comprises the amino acid sequence of SEQ ID NO: 7, or an amino acid sequence having at least about 85%, 90%, 95%, or 99% sequence identity thereof. In some embodiments, the nucleic acid molecule comprises a nucleic acid sequence encoding the costimulatory signaling domain, wherein the nucleic acid sequence comprises the nucleic acid sequence of SEQ ID NO: 18, or a nucleic acid sequence having at least about 85%, 90%, 95%, or 99% sequence identity thereof.

In some embodiments, the intracellular signaling domain comprises a functional signaling domain derived from 4-1BB and a functional signaling domain derived from CD3 zeta. In some embodiments, the intracellular signaling domain comprises the amino acid sequence of SEQ ID NO: 7 (or an amino acid sequence having at least about 85%, 90%, 95%, or 99% sequence identity thereof) and the amino acid sequence of SEQ ID NO: 9 or 10 (or an amino acid sequence having at least about 85%, 90%, 95%, or 99% sequence identity thereof). In some embodiments, the intracellular signaling domain comprises the amino acid sequence of SEQ ID NO: 7 and the amino acid sequence of SEQ ID NO: 9 or 10.

In some embodiments, the CAR further comprises a leader sequence comprising the amino acid sequence of SEQ ID NO: 1.

In some embodiments, this invention features a population of CAR-expressing cells (for example, autologous or allogeneic CAR-expressing T cells or NK cells) made by any of the aforementioned methods or any other method disclosed herein. In some embodiments, disclosed herein is a pharmaceutical composition comprising a population of CAR-expressing cells disclosed herein and a pharmaceutically acceptable carrier.

### In Some Embodiments, the Population Comprises:

(a) a first population of cells comprising an anti-BCMA CAR but not an anti-CD19 CAR;
(b) a second population of cells comprising an anti-CD19 CAR but not an anti-BCMA CAR; and
(c) a third population of cells comprising both an anti-BCMA CAR and an anti-CD19 CAR.

### In Some Embodiments:

(i) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined;
(ii) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined; and/or
(iii) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the population.

In some embodiments, the population further comprises a fourth population of cells that do not comprise a CAR.

### In Some Embodiments:

(i) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined;
(ii) the total number of viable cells in the second population is less than or equal to: about 45% to about 50% (e.g., about 47%); about 50 to about 55% (e.g., about 53%); about 60% to about 65% (e.g., about 63%); or about 80 to about 85% (e.g., about 82%) of the total number of viable cells in the first and third populations combined.

In some embodiments, in the final CAR cell product manufactured using the methods described herein, the total amount of beads (e.g., CD4 beads, CD8 beads, and/or TransACT beads) is no more than 0.04, 0.05, 0.06, 0.07, 0.08, 0.09, 0.1, 0.2, 0.3, 0.4, or 0.5% of the total amount of beads added during the manufacturing process.

In some embodiments, this invention features a population of CAR-expressing cells (for example, autologous or allogeneic CAR-expressing T cells or NK cells) comprising one or more of the following characteristics: (a) about the same percentage of naïve cells, for example, naïve T cells, for example, CD45RO− CCR7+ T cells, as compared to the percentage of naïve cells, for example, naïve T cells, for example, CD45RO− CCR7+ cells, in the same population of cells prior to being engineered to express the CAR; (b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to express the CAR; (c) an increased percentage of naïve cells, for example, naïve T cells, for example, CD45RO− CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to express the CAR; (d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR; (f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to express the CAR; (g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; (h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; or (i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR.

In some embodiments, this invention features a population of CAR-expressing cells (for example, autologous or allogeneic CAR-expressing T cells or NK cells), wherein: (a) the median GeneSetScore (Up TEM vs. Down TSCM) of the population of cells is about the same as or differs by no more than (for example, increased by no more than) about 25, 50, 75, 100, or 125% from the median GeneSetScore (Up TEM vs. Down TSCM) of the same population of cells prior to being engineered to express the CAR; (b) the median GeneSetScore (Up Treg vs. Down Teff) of the population of cells is about the same as or differs by no more than (for example, increased by no more than) about 25, 50, 100, 150, or 200% from the median GeneSetScore (Up Treg vs. Down Teff) of the population of cells prior to being engineered to express the CAR; (c) the median GeneSetScore (Down stemness) of the population of cells is about the same as or differs by no more than (for example, increased by no more than) about 25, 50, 100, 150, 200, or 250% from the median GeneSetScore (Down stemness) of the population of cells prior to being engineered to express the CAR; (d) the median GeneSetScore (Up hypoxia) of the population of cells is about the same as or differs by no more than (for example, increased by no more than) about 125, 150, 175, or 200% from the median GeneSetScore (Up hypoxia) of the population of cells prior to being engineered to express the CAR; or (e) the median GeneSetScore (Up autophagy) of the population of cells is about the same as or differs by no more than (for example, increased by no more than) about 180, 190, 200, or 210% from the median GeneSetScore (Up autophagy) of the population of cells prior to being engineered to express the CAR.

In some embodiments, this invention features a method of increasing an immune response in a subject, comprising administering a population of CAR-expressing cells disclosed herein or a pharmaceutical composition disclosed herein to the subject, thereby increasing an immune response in the subject.

In some embodiments, disclosed herein is a method of treating a cancer in a subject, comprising administering a population of CAR-expressing cells disclosed herein or a pharmaceutical composition disclosed herein to the subject, thereby treating the cancer in the subject. In some embodiments, the cancer is a solid cancer, for example, chosen from: one or more of mesothelioma, malignant pleural mesothelioma, non-small cell lung cancer, small cell lung cancer, squamous cell lung cancer, large cell lung cancer, pancreatic cancer, pancreatic ductal adenocarcinoma, esophageal adenocarcinoma, breast cancer, glioblastoma, ovarian cancer, colorectal cancer, prostate cancer, cervical cancer, skin cancer, melanoma, renal cancer, liver cancer, brain cancer, thymoma, sarcoma, carcinoma, uterine cancer, kidney cancer, gastrointestinal cancer, urothelial cancer, pharynx cancer, head and neck cancer, rectal cancer, esophagus cancer, or bladder cancer, or a metastasis thereof. In some embodiments, the cancer is a liquid cancer, for example, chosen from: chronic lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma.

In some embodiments, the method further comprises administering a second therapeutic agent to the subject. In some embodiments, the second therapeutic agent is an anti-cancer therapeutic agent, for example, a chemotherapy, a radiation therapy, or an immune-regulatory therapy. In some embodiments, the second therapeutic agent is IL-15 (for example, hetIL-15 (IL15/sIL-15Ra)).

In some embodiments, provided herein is an isolated cell or a population of cells made by a method as described herein comprising one or more cells comprising:
(a) a first nucleic acid molecule encoding a first CAR that comprises an anti-BCMA binding domain, a first transmembrane domain, and a first intracellular signaling domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of SEQ ID NOs: 86, 87, 88, 95, 96, and 97, respectively; and
(b) a second nucleic acid molecule encoding a second CAR that comprises an anti-CD19 binding domain, a second transmembrane domain, and a second intracellular signaling domain, wherein the anti-CD19 binding domain comprises a VH comprising a HC CDR1, a HC CDR2, and a HC CDR3, and a VL comprising a LC CDR1, a LC CDR2, and a LC CDR3, wherein the HC CDR1, HC CDR2, HC CDR3, LC CDR1, LC CDR2, and LC CDR3 comprise the amino acid sequences of SEQ ID NOs: 295, 304, and 297-300, respectively.

### In Some Embodiments, Provided Herein is an Isolated Cell Comprising:

(a) a first nucleic acid molecule encoding a first CAR that comprises an anti-BCMA binding domain, a first transmembrane domain, and a first intracellular signaling domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of SEQ ID NOs: 86, 87, 88, 95, 96, and 97, respectively; and
(b) a second nucleic acid molecule encoding a second CAR that comprises an anti-CD19 binding domain, a second transmembrane domain, and a second intracellular signaling domain, wherein the anti-CD19 binding domain comprises a VH comprising a HC CDR1, a HC CDR2, and a HC CDR3, and a VL comprising a LC CDR1, a LC CDR2, and a LC CDR3, wherein the HC CDR1, HC CDR2, HC CDR3, LC CDR1, LC CDR2, and LC CDR3 comprise the amino acid sequences of SEQ ID NOs: 295, 304, and 297-300, respectively.

In some embodiments, the VH and VL of the anti-BCMA binding domain comprise the amino acid sequences of SEQ ID NOs: 93 and 102, respectively. In some embodiments, the VH and VL of the anti-CD19 binding domain comprise the amino acid sequences of SEQ ID NOs: 250 and 251, respectively. In some embodiments, the VH and VL of the anti-BCMA binding domain comprise the amino acid sequences of SEQ ID NOs: 93 and 102, respectively, and the VH and VL of the anti-CD19 binding domain comprise the amino acid sequences of SEQ ID NOs: 250 and 251, respectively. In some embodiments, the anti-BCMA binding domain comprises the amino acid sequence of SEQ ID NO: 105. In some embodiments, the anti-CD19 binding domain comprises the amino acid sequence of SEQ ID NO: 293. In some embodiments, the anti-BCMA binding domain comprises the amino acid sequence of SEQ ID NO: 105 and the anti-CD19 binding domain comprises the amino acid sequence of SEQ ID NO: 293. In some embodiments, the first CAR comprises the amino acid sequence of SEQ ID NO: 107. In some embodiments, the second CAR comprise the amino acid sequence of SEQ ID NO: 225. In some embodiments, the first CAR comprises the amino acid sequence of SEQ ID NO: 107; and the second CAR comprise the amino acid sequence of SEQ ID NO: 225. In some embodiments, the first CAR is encoded by the nucleic acid sequence of SEQ ID NO: 259, 258, or 416. In some embodiments, the second CAR is encoded by the nucleic acid sequence of SEQ ID NO: 417, 355, 356, or 354. In some embodiments, the first CAR is encoded by the nucleic acid sequence of SEQ ID NO: 259, 258, or 416, and the second CAR is encoded by the nucleic acid sequence of SEQ ID NO: 417, 355, 356, or 354.

In some embodiments, provided herein is a pharmaceutical composition comprising the cell or population of cells, as described herein.

In some embodiments, provided herein is method of providing anti-tumor immunity in a subject or treating a subject having a disease associated with expression of BCMA comprising administering to the subject an effective amount of the cell or population of cells or the pharmaceutical composition, as described herein.

In some embodiments, the disease associated with BCMA expression is a hematologic cancer or a solid cancer, e.g., a hematologic cancer or a solid cancer described herein.

In some embodiments, the disease is chosen from: acute leukemia, B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell- or large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, prostate cancer (e.g., castrate-resistant or therapy-resistant prostate cancer, or metastatic prostate cancer), pancreatic cancer, lung cancer, a plasma cell proliferative disorder (e.g., asymptomatic myeloma (smoldering multiple myeloma or indolent myeloma), monoclonal gammapathy of undetermined significance (MGUS), Waldenstrom's macroglobulinemia, plasmacytoma (e.g., plasma cell dyscrasia, solitary myeloma, solitary plasmacytoma, extramedullary plasmacytoma, and multiple plasmacytoma), systemic amyloid light chain amyloidosis, or POEMS syndrome (also known as Crow-Fukase syndrome, Takatsuki disease, and PEP syndrome)), or a combination thereof.

In some embodiments, the disease is multiple myeloma.

Although methods and materials similar or equivalent to those described herein can be used in the practice or testing of the present invention, suitable methods and materials are described below. All publications, patent applications, patents, and other references (for example, sequence database reference numbers) mentioned herein are incorporated by reference in their entirety. For example, all GenBank, Unigene, and Entrez sequences referred to herein, for example, in any Table herein, are incorporated by reference. When one gene or protein references a plurality of sequence accession numbers, all of the sequence variants are encompassed.

In addition, the materials, methods, and examples are illustrative only and not intended to be limiting. Headings, sub-headings or numbered or lettered elements, for example, (a), (b), (i) etc., are presented merely for ease of reading. The use of headings or numbered or lettered elements in this document does not require the steps or elements be performed in alphabetical order or that the steps or elements are necessarily discrete from one another. Other features, objects, and advantages of the invention will be apparent from the description and drawings, and from the claims.

### BRIEF DESCRIPTION OF THE FIGURES

FIGS. 1A-1H: Jurkat NFAT Luciferase (JNL) reporter assay using an automated system was used to test the function of BCMA CARs. CAR clones were evaluated in the JNL reporter assay for antigen-dependent activity. JNL cells containing the indicated CAR clones or untransduced JNL cells (UTD) were co-cultured with media alone (FIGS. 1G and 1H) or with target cells lines (KMS11 as a BCMA-positive cell line (FIGS. 1A and 1C) and NALM6 as a BCMA-negative cell line (FIGS. 1E and 1F)) at different ratios and luciferase activity was measured as luminescence intensity. Clones were considered active when the luminescence intensity exceeded 2-fold the level of UTD cells in the presence of antigen-expressing cells. Luminescence read-out is a direct measurement of CAR stimulation. FIGS. 1B and 1D are graphs showing expression level of BCMA CARs on JNL cells were detected by flow cytometry using a human recombinant (r)BCMA_Fc-AF647. 1× or 2× platform indicated 40,000 of H293 cells or 80,000 of H293 cells seeded for viral production.

FIG. 2: Expression level of BCMA CARs on primary human T cells. Cells were stained with a human rBCMA_Fc-AF647 reagent and assayed by flow cytometry. The percentage of CAR+ cells and MFI are shown in the graph for day 5 and day 9 of cell culture. Data is summarized in Table 27, which includes the viral titer achieved for the respective CARs.

FIGS. 3A-3C: The ability of T cells expressing the indicated CARs to mediate cell lysis and cytokine production were evaluated against the KMS11 target cell line expressing firefly luciferase (KMS11-luc). FIG. 3A: CART cells were co-cultured with KMS11-luc target cells at the indicated E:T ratios. % cell killing was determined by the difference in luciferase signal between target cells without effector T cells (control) and with effector T cells (experimental), expressed as a percent of the control. UTD represents untransduced T cells. FIG. 3B: Background killing was observed for the BCMA-negative line NALM6. FIG. 3C: IFNγ was measured by MSD in the supernatants collected at 24 h from these co-culture systems with a E:T ratio of 2.5. All data is expressed as the average +/−standard deviation.

FIGS. 4A-4C: CAR expression in T cells transduced with a MOI=5 (viral titer defined by the first CAR expressed in SupT1 cells). FIG. 4A is a table summarizing % CAR19, % BCMA CAR, % Double Positive, % CAR19-only, and % BCMA-CAR-only of different constructs. FIG. 4B is a set of flow cytometry plots showing the staining of cells for surface BCMA CAR expression (x-axis) and surface CD19 CAR expression (y-axis). FIG. 4C is a pair of bar graphs showing BCMA CAR MFI (upper panel) and CD19 CAR MFI (lower panel).

FIGS. 5A-5C: In vitro killing assay using Day 8 CART cells. FIGS. 5A-5C are a set of graphs showing % Killing against BCMA-positive KMS11 cells, CD19-positive Nalm6 cells, or BCMA/CD19-negative cells, respectively, at the indicated E:T ratios.

FIGS. 6A-6D: In vitro cytokine production using Day 8 CART cells. FIGS. 6A-6D are a set of bar graphs showing IFN gamma production of CART cells when co-cultured with BCMA-positive KMS11 cells or CD19-positive Nalm6 cells.

FIGS. 7A-7C: Individual CAR expression of cells manufactured using the ARM process. FIGS. 7A-7B are histograms showing the expression pattern of both anti-BCMA and anti-CD19 CARs at 24 h or 72 h post-transduction of human primary T cells manufactured using the ARM process. The studies used a MOI of 1 based on the SupT1 titer determined by expression of the upstream CAR. In each of FIGS. 7A and 7B, the left part is a panel of histograms showing staining using rBCMA-Fc, and the right part is a panel of histograms showing staining using anti-idiotype antibody that binds to CD19 CAR. Constructs #244 (“c244”) and #245 (“c245”) are mono anti-CD19 CAR and mono anti-BCMA CAR, respectively. FIG. 7C is a panel of flow cytometry plots showing the anti-BCMA and anti-CD19 CAR expression pattern at 72 h post-transduction of human primary T cells using a MOI of 1 based on the upstream CAR titer.

FIGS. 8A-8C: In vivo anti-tumor activity of construct #236 (“c236”) and construct #238 (“c238”) using three mouse models: a disseminated KMS-11(BCMA+CD19−) multiple myeloma model, expressing a luciferase reporter gene (KMS11-Luc) (FIG. 8A), a Nalm6-Luc (CD19+BCMA−) xenograft mouse model (FIG. 8B) and a mixed model of 95% KMS-luc with 5% NALM6-Luc cells (FIG. 8C). The tumor burden is expressed as total body luminescence (p/s), depicted as mean tumor burden+SEM. On day 7 or 8 post tumor inoculation, mice were treated with c236 and c238 at designated doses of BCMA CAR+ or CD19 CAR+ T cell (approximate number of viable CAR+ T cells), as shown in Table 30. Vehicle (PBS) and non-transduced T cells (UTD) served as negative controls. Mono anti-BCMA CAR P161 and mono anti-CD19 CAR CTL119 were also used as controls.

FIGS. 9A-9C: Body weight loss induced by graft-versus-host response. All mice were individually monitored for body weight loss, as a read-out for X-GvHD by measuring body weight over time. Body weight (BWT) is plotted as % change from baseline.

FIGS. 10A-10C: In vivo expansion of peripheral blood CD3+ T cells was analyzed by flow cytometry up to 4 weeks after infusion.

FIGS. 11A-11C: In vivo expansion of CAR+ T cells (BCMA CAR+ percentage) was analyzed by flow cytometry up to 4 weeks after infusion.

FIGS. 12A-12C: In vivo expansion of CAR+ T cells (double CAR+ counts) was analyzed by flow cytometry up to 4 weeks after infusion.

FIGS. 13A-13C: In vivo plasma IFN-γ kinetics. Plasma IFN-γlevels from all three mouse models treated with c236 and c238, as well as monoCAR controls, at respective CAR-T doses are plotted in the graphs. Mice were bled and plasma cytokine measured by MSD assay.

FIGS. 14A and 14B: In vivo efficacy and cellular expansion of cells generated using 236 and c238 in a multiple myeloma xenograft mouse model. FIG. 14A: NSG mice were injected with multiple myeloma cell line KMS11, which expressed a luciferase reporter gene. The tumor burden is expressed as total body luminescence (p/s), depicted as mean tumor burden+SEM. On day 8 post tumor inoculation, mice were treated with c236 and c238 at 9e4 BCMA-CD19 double CAR+ T cell dose (approximate number of viable CAR+ T cells). Vehicle (PBS) and non-transduced T cells (UTD) served as negative controls. FIG. 14B: The expansion of peripheral blood CAR+ T cells was analyzed by flow cytometry up to 4 weeks after infusion. Double anti-BCMA and CD19 CAR+ T cell expansion was observed in c236 and c238 CAR-T Rx groups.

FIGS. 15A and 15B: CAR expression of cells manufactured using the ARM process. Flow cytometry plots showing the expression of double positive anti-BCMA and anti-CD19 CARs at 96 h (FIG. 15A) and 7 days (FIG. 15B) post viral addition to human primary T cells manufactured using the ARM process. The studies used a MOI of 2 based on the SupT1 titer determined by expression of double CAR (positive for PI61 or R1G5 clones and CTL119) detected by anti-idiotype antibody that binds to CD19CAR and recombinant BCMA_Fc (AF647) that binds to PI61 or R1G5. Mono anti-BCMA CARTs PI61 and R1G5, and mono anti-CD19 CART CTL119 served as controls.

FIG. 16: CAR Expression at day 7 with TM process using MOI of 5. Flow cytometry plots showing the expression of double positive anti-BCMA and anti-CD19 CARs on day 7 post viral addition to human primary T cells manufactured using the TM process. The studies used a MOI of 5 based on the SupT1 titer determined by expression of double CAR (positive for PI61 or R1G5 clones and CTL119) detected by anti-idiotype antibody that binds to CD19CAR and recombinant BCMA_Fc (AF647) that binds to P161 or R1G5.

FIGS. 17A and 17B: In vitro specific killing of BCMA- or CD19-expressing tumor cells by T cells engineered with anti-BCMACAR and CD19CAR diabody constructs. The ability of T cells expressing PI61/CTL119 clones to mediate cell lysis was evaluated against the KMS11-Luc or NALM6-Luc target cell line. CART cells were co-cultured with BCMA+KMS-11-luc or BCMA-NALM6-Luc target cells at the indicated E:T ratios for 20 h, and % cell killing, determined by the difference in luciferase signal between target cells without effector T cells (control) and with effector T cells (experimental) expressed as a percent of the control, was measured as a surrogate for target cell lysis. UTD represents untransduced T cells. Mono PI61 or CTL119 served as controls.

FIGS. 18A and 18B: Cytokine production of T cells engineered with anti-BCMACAR and CD19CAR diabody constructs in response to BCMA- or CD19-expressing tumor cells. IFN-γ(FIG. 18A) and IL-2 (FIG. 18B) were measured by MSD in the supernatants from the killing assay co-culture at a ratio of 1.25:1.

FIG. 19: Percentages of the double CAR positive population, BCMA CAR positive population, and CD19 CAR positive population on Day 4.

FIG. 20: Flow cytometry plots showing staining of cells with rBCMA-Fc and an anti-idiotype antibody that binds to CD19 CAR.

FIG. 21: Percentages of total CAR positive populations on Day 4 and Day 7 under the indicated conditions.

FIG. 22: Cell counts (left panel) and percentage of live cells (right panel) on Days 0, 1, 3, and 7 under the indicated conditions.

FIGS. 23A, 23B, and 23C: Single cell RNA-seq data for input cells (FIG. 23A), Day 1 cells (FIG. 23B), and Day 9 cells (FIG. 23C). The “nGene” graphs show the number of expressed genes per cell. The “nUMI” graphs show the number of unique molecular identifiers (UMIs) per cell.

FIGS. 24A, 24B, 24C, and 24D: T-Distributed Stochastic Neighbor Embedding (TSNE) plots comparing input cells (FIG. 24A), Day 1 cells (FIG. 24B), and Day 9 cells (FIG. 24C) for a proliferation signature, which was determined based on expression of genes CCNB1, CCND1, CCNE1, PLK1, and MKI67. Each dot represents a cell in that sample. Cells shown as light grey do not express the proliferation genes whereas dark shaded cells express one or more of the proliferation genes. FIG. 24D is a violin plot showing the distribution of gene set scores for a gene set comprised of genes that characterize a resting vs. activated T cell state for Day 1 cells, Day 9 cells, and input cells. In FIG. 24D, a higher gene set score (Up resting vs. Down activated) indicates an increasing resting T cell phenotype, whereas a lower gene set score (Up resting vs. Down activated) indicates an increasing activated T cell phenotype. Input cells were overall in more of a resting state compared to Day 9 and Day 1 cells. Day 1 cells show the greatest activation gene set score.

FIGS. 25A, 25B, 25C, 25D and 25E: Gene set analysis for input cells, Day 1 cells, and Day 9 cells. In FIG. 25A, a higher gene set score for the gene set “Up TEM vs. Down TSCM” indicates an increasing effector memory T cell (TEM) phenotype of the cells in that sample, whereas a lower gene set score indicates an increasing stem cell memory T cell (TSCM) phenotype. In FIG. 25B, a higher gene set score for the gene set “Up Treg vs. Down Teff” indicates an increasing regulatory T cell (Treg) phenotype, whereas a lower gene set score indicates an increasing effector T cell (Teff) phenotype. In FIG. 25C, a lower gene set score for the gene set “Down stemness” indicates an increasing stemness phenotype. In FIG. 25D, a higher gene set score for the gene set “Up hypoxia” indicates an increasing hypoxia phenotype. In FIG. 25E, a higher gene set score for the gene set “Up autophagy” indicates an increasing autophagy phenotype. Day 1 cells looked similar to the input cells in terms of memory, stem-like and differentiation signature. Day 9 cells, on the other hand, show a higher enrichment for metabolic stress.

FIGS. 26A, 26B, and 26C: Gene cluster analysis for input cells. FIGS. 26A-26C are violin plots showing the gene set scores from gene set analysis of the four clusters of the input cells. Each dot overlaying the violin plots in FIGS. 26A-26C represents a cell's gene set score. In FIG. 26A, a higher gene set score of the gene set “Up Treg vs. Down Teff” indicates an increasing Treg cell phenotype, whereas a lower gene set score of the gene set “Up Treg vs. Down Teff” indicates an increasing Teff cell phenotype. In FIG. 26B, a higher gene set score of the gene set “Progressively up in memory differentiation” indicates an increasing late memory T cell phenotype, whereas a lower gene set score of the gene set “Progressively up in memory differentiation” indicates an increasing early memory T cell phenotype. In FIG. 26C, a higher gene set score of the gene set “Up TEM vs. Down TN” indicates an increasing effector memory T cell phenotype, whereas a lower gene set score of the gene set “Up TEM vs. Down TN” indicates an increasing naïve T cell phenotype. The cells in Cluster 3 are shown to be in a later memory, further differentiated T cell state compared to the cells in Cluster 1 and Cluster 2 which are in an early memory, less differentiated T cell state. Cluster 0 appears to be in an intermediate T cell state. Taken together, this data shows that there is a considerable level of heterogeneity within input cells.

FIGS. 27A, 27B, and 27C: TCR sequencing and measuring clonotype diversity. Day 9 cells have flatter distribution of clonotype frequencies (higher diversity).

FIGS. 28A and 28B: Flow cytometry analyses for CAR expression on days 4 and 7 post-transduction. Flow cytometry analyses for CAR-T cells generated by co-transducing cells with BCMA and CD19CAR vectors at different combinations of MOIs with ARM process in a 24-well plate. FIG. 28A: Flow cytometry plots showed mono anti-BCMA CAR, mono anti-CD19 CAR and double+ CAR expression on days 4 and 7 post-transduction under four different MOI conditions in addition to controls (UTD and single vector). FIG. 28B: Quantification of subsets of CAR+ populations including total anti-BCMA CAR+ T cells, total anti-CD19 CAR+ T cells as well as total CAR+ T cells (sum of the two mono CAR+ T cells and double+ CAR T cells) in each condition as described in FIG. 28A. Data shown are one representative from three donor T cells with consistent results. CAR+ cell percentages are gated on live CD3+ T cell population.

FIG. 29: Flow cytometry analyses for CAR expression on day 4 post-transduction. Flow cytometry analyses of final products of dual targeting cocktail CART, mono BCMACART and mono CD19CART for CAR expression on day 4 post-transduction. A small aliquots of each product at 24 h harvest were re-cultured for three days prior to flow cytometry staining.

FIGS. 30A, 30B, 30C, 30D, and 30E: In vivo efficacy of dual CART compared to mono BCMA CART and CD19 CART in xenograft models. NSG mice were injected with cell lines expressing the luciferase reporter gene (KMS-11, or Nalm-6, or a mix of both with 5% of Nalm-6-luc). The tumor burden is expressed as total body luminescence (p/s), depicted as mean tumor burden+SEM. On day 7 or 8 post tumor inoculation, mice were treated with dual targeting cocktail CART, BCMA CART, or CD19 CART at the respective doses (approximate number of viable CAR+ T cells). Vehicle (PBS) and non-transduced T cells (UTD) served as negative controls. N=5 mice for all groups. BCMACART and CD19CART served as respective controls using the highest dose level. All experiments were terminated on day 23 after CAR-T administration.

FIG. 31: FIG. 31 is a bar graph showing % mono CD19 CAR+ cells, % mono BCMA CAR+ cells, and % double BCMA/CD19 CAR+ cells on day 4 post transduction (day 3 past harvest).

FIG. 32: Characterization of T cell subsets. FIG. 32 is a graph showing % CD4+ T cells, CD8+ T cells, naïve T cells (Tn), central memory T cells (Tcm), effector memory T cells (Tem), and effector memory T cells re-expressing CD45RA (Temra), in the input material, post-enrichment material, and Day 1 Post-harvest material.

FIGS. 33A and 33B: Plasma IFN-γ Kinetics of BCMA/CD19 dual CART cellular product, BCMA CART, and CD19 CART treated mice. Animals were treated with PBS, UTD, BCMA/CD19 dual CART cellular product, BCMA CART, or CD19 CART at respective CAR-T doses. Mice were bled and plasma cytokine measured by MSD assay.

### DETAILED DESCRIPTION

### Definitions

Unless defined otherwise, all technical and scientific terms used herein have the same meaning as commonly understood by one of ordinary skill in the art to which the invention pertains.

The term “a” and “an” refers to one or to more than one (i.e., to at least one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The compositions and methods of the present invention encompass polypeptides and nucleic acids having the sequences specified, or sequences substantially identical or similar thereto, for example, sequences at least 85%, 90%, or 95% identical or higher to the sequence specified. In the context of an amino acid sequence, the term “substantially identical” is used herein to refer to a first amino acid sequence that contains a sufficient or minimum number of amino acid residues that are i) identical to, or ii) conservative substitutions of aligned amino acid residues in a second amino acid sequence such that the first and second amino acid sequences can have a common structural domain and/or common functional activity, for example, amino acid sequences that contain a common structural domain having at least about 85%, 90%, 91%, 92%, 93%, 94%, 95%, 96%, 97%, 98% or 99% identity to a reference sequence, for example, a sequence provided herein.

In the context of a nucleotide sequence, the term “substantially identical” is used herein to refer to a first nucleic acid sequence that contains a sufficient or minimum number of nucleotides that are identical to aligned nucleotides in a second nucleic acid sequence such that the first and second nucleotide sequences encode a polypeptide having common functional activity, or encode a common structural polypeptide domain or a common functional polypeptide activity, for example, nucleotide sequences having at least about 75%, 80%, 85%, 90%, 91%, 92%, 93%, 94%, 95%, 96%, 97%, 98% or 99% identity to a reference sequence, for example, a sequence provided herein.

The term “variant” refers to a polypeptide that has a substantially identical amino acid sequence to a reference amino acid sequence, or is encoded by a substantially identical nucleotide sequence. In some embodiments, the variant is a functional variant.

The term “functional variant” refers to a polypeptide that has a substantially identical amino acid sequence to a reference amino acid sequence, or is encoded by a substantially identical nucleotide sequence, and is capable of having one or more activities of the reference amino acid sequence.

The term cytokine (for example, IL-2, IL-7, IL-15, IL-21, or IL-6) includes full length, a fragment or a variant, for example, a functional variant, of a naturally-occurring cytokine (including fragments and functional variants thereof having at least 10%, 30%, 50%, or 80% of the activity, e.g., the immunomodulatory activity, of the naturally-occurring cytokine). In some embodiments, the cytokine has an amino acid sequence that is substantially identical (e.g., at least about 85%, 90%, 91%, 92%, 93%, 94%, 95%, 96%, 97%, 98% or 99% identity) to a naturally-occurring cytokine, or is encoded by a nucleotide sequence that is substantially identical (e.g., at least about 75%, 80%, 85%, 90%, 91%, 92%, 93%, 94%, 95%, 96%, 97%, 98% or 99% identity) to a naturally-occurring nucleotide sequence encoding a cytokine. In some embodiments, as understood in context, the cytokine further comprises a receptor domain, e.g., a cytokine receptor domain (e.g., an IL-15/IL-15R).

As used herein, the term “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells, and plasma cells. Its ligand is called B-cell activator of the TNF family (BAFF) and a proliferation inducing ligand (APRIL). BCMA is involved in mediating the survival of plasma cells for maintaining long-term humoral immunity. The gene for BCMA is encoded on chromosome 16 producing a primary mRNA transcript of 994 nucleotides in length (NCBI accession NM_001192.2) that encodes a protein of 184 amino acids (NP_001183.2). A second antisense transcript derived from the BCMA locus has been described, which may play a role in regulating BCMA expression. (Laabi Y. et al., Nucleic Acids Res., 1994, 22:1147-1154). Additional transcript variants have been described with unknown significance (Smirnova A S et al. Mol Immunol., 2008, 45(4):1179-1183. A second isoform, also known as TV4, has been identified (Uniprot identifier Q02223-2). As used herein, “BCMA” includes proteins comprising mutations, e.g., point mutations, fragments, insertions, deletions and splice variants of full length wild-type BCMA.

The phrase “disease associated with expression of BCMA” includes, but is not limited to, a disease associated with a cell which expresses BCMA (e.g., wild-type or mutant BCMA) or condition associated with a cell which expresses BCMA (e.g., wild-type or mutant BCMA) including, e.g., proliferative diseases such as a cancer or malignancy or a precancerous condition such as a myelodysplasia, a myelodysplastic syndrome or a preleukemia; or a noncancer related indication associated with a cell which expresses BCMA (e.g., wild-type or mutant BCMA). For the avoidance of doubt, a disease associated with expression of BCMA may include a condition associated with a cell which does not presently express BCMA, e.g., because BCMA expression has been downregulated, e.g., due to treatment with a molecule targeting BCMA, e.g., a BCMA inhibitor described herein, but which at one time expressed BCMA. In one aspect, a cancer associated with expression of BCMA (e.g., wild-type or mutant BCMA) is a hematological cancer. In one aspect, the hematological cancer is a leukemia or a lymphoma. In one aspect, a cancer associated with expression of BCMA (e.g., wild-type or mutant BCMA) is a malignancy of differentiated plasma B cells. In one aspect, a cancer associated with expression of BCMA(e.g., wild-type or mutant BCMA) includes cancers and malignancies including, but not limited to, e.g., one or more acute leukemias including but not limited to, e.g., B-cell acute Lymphoid Leukemia (“BALL”), T-cell acute Lymphoid Leukemia (“TALL”), acute lymphoid leukemia (ALL); one or more chronic leukemias including but not limited to, e.g., chronic myelogenous leukemia (CML), Chronic Lymphoid Leukemia (CLL). Additional cancers or hematologic conditions associated with expression of BMCA (e.g., wild-type or mutant BCMA) comprise, but are not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. In some embodiments, the cancer is multiple myeloma, Hodgkin's lymphoma, non-Hodgkin's lymphoma, or glioblastoma. In embodiments, a disease associated with expression of BCMA includes a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). Further diseases associated with expression of BCMA (e.g., wild-type or mutant BCMA) expression include, but not limited to, e.g., atypical and/or non-classical cancers, malignancies, precancerous conditions or proliferative diseases associated with expression of BCMA (e.g., wild-type or mutant BCMA), e.g., a cancer described herein, e.g., a prostate cancer (e.g., castrate-resistant or therapy-resistant prostate cancer, or metastatic prostate cancer), pancreatic cancer, or lung cancer.

Non-cancer related conditions that are associated with BCMA (e.g., wild-type or mutant BCMA) include viral infections; e.g., HIV, fungal infections, e.g., C. neoformans; autoimmune disease; e.g. rheumatoid arthritis, system lupus erythematosus (SLE or lupus), pemphigus vulgaris, and Sjogren's syndrome; inflammatory bowel disease, ulcerative colitis; transplant-related all specific immunity disorders related to mucosal immunity; and unwanted immune responses towards biologics (e.g., Factor VIII) where humoral immunity is important. In embodiments, a non-cancer related indication associated with expression of BCMA includes but is not limited to, e.g., autoimmune disease, (e.g., lupus), inflammatory disorders (allergy and asthma) and transplantation. In some embodiments, the tumor antigen-expressing cell expresses, or at any time expressed, mRNA encoding the tumor antigen. In an embodiment, the tumor antigen-expressing cell produces the tumor antigen protein (e.g., wild-type or mutant), and the tumor antigen protein may be present at normal levels or reduced levels. In an embodiment, the tumor antigen-expressing cell produced detectable levels of a tumor antigen protein at one point, and subsequently produced substantially no detectable tumor antigen protein.

The term “Chimeric Antigen Receptor” or alternatively a “CAR” or “CAR molecule” refers to a recombinant polypeptide construct comprising at least an extracellular antigen binding domain, a transmembrane domain and a cytoplasmic signaling domain (also referred to herein as “an intracellular signaling domain”) comprising a functional signaling domain derived from a stimulatory molecule as defined below. In some embodiments, the domains in the CAR polypeptide construct are in the same polypeptide chain, for example, comprise a chimeric fusion protein. In some embodiments, the domains in the CAR polypeptide construct are not contiguous with each other, for example, are in different polypeptide chains, for example, as provided in an RCAR as described herein.

In some embodiments, the cytoplasmic signaling domain comprises a primary signaling domain (for example, a primary signaling domain of CD3-zeta). In some embodiments, the cytoplasmic signaling domain further comprises one or more functional signaling domains derived from at least one costimulatory molecule as defined below. In some embodiments, the costimulatory molecule is chosen from 41BB (i.e., CD137), CD27, ICOS, and/or CD28. In some embodiments, the CAR comprises a chimeric fusion protein comprising an extracellular antigen recognition domain, a transmembrane domain and an intracellular signaling domain comprising a functional signaling domain derived from a stimulatory molecule. In some embodiments, the CAR comprises a chimeric fusion protein comprising an extracellular antigen recognition domain, a transmembrane domain and an intracellular signaling domain comprising a functional signaling domain derived from a costimulatory molecule and a functional signaling domain derived from a stimulatory molecule. In some embodiments, the CAR comprises a chimeric fusion protein comprising an extracellular antigen recognition domain, a transmembrane domain and an intracellular signaling domain comprising two functional signaling domains derived from one or more costimulatory molecule(s) and a functional signaling domain derived from a stimulatory molecule. In some embodiments, the CAR comprises a chimeric fusion protein comprising an extracellular antigen recognition domain, a transmembrane domain and an intracellular signaling domain comprising at least two functional signaling domains derived from one or more costimulatory molecule(s) and a functional signaling domain derived from a stimulatory molecule. In some embodiments the CAR comprises an optional leader sequence at the amino-terminus (N-terminus) of the CAR fusion protein. In some embodiments, the CAR further comprises a leader sequence at the N-terminus of the extracellular antigen recognition domain, wherein the leader sequence is optionally cleaved from the antigen recognition domain (for example, an scFv) during cellular processing and localization of the CAR to the cellular membrane.

A CAR that comprises an antigen binding domain (for example, an scFv, a single domain antibody, or TCR (for example, a TCR alpha binding domain or TCR beta binding domain)) that targets a specific tumor marker X, wherein X can be a tumor marker as described herein, is also referred to as XCAR. For example, a CAR that comprises an antigen binding domain that targets BCMA is referred to as BCMA CAR. The CAR can be expressed in any cell, for example, an immune effector cell as described herein (for example, a T cell or an NK cell).

The term “signaling domain” refers to the functional portion of a protein which acts by transmitting information within the cell to regulate cellular activity via defined signaling pathways by generating second messengers or functioning as effectors by responding to such messengers.

The term “antibody,” as used herein, refers to a protein, or polypeptide sequence derived from an immunoglobulin molecule, which specifically binds with an antigen. Antibodies can be polyclonal or monoclonal, multiple or single chain, or intact immunoglobulins, and may be derived from natural sources or from recombinant sources. Antibodies can be tetramers of immunoglobulin molecules.

The term “antibody fragment” refers to at least one portion of an intact antibody, or recombinant variants thereof, and refers to the antigen binding domain, for example, an antigenic determining variable region of an intact antibody, that is sufficient to confer recognition and specific binding of the antibody fragment to a target, such as an antigen. Examples of antibody fragments include, but are not limited to, Fab, Fab′, F(ab′)2, and Fv fragments, scFv antibody fragments, linear antibodies, single domain antibodies such as sdAb (either VL or VH), camelid VHH domains, and multi-specific molecules formed from antibody fragments such as a bivalent fragment comprising two or more, for example, two, Fab fragments linked by a disulfide bridge at the hinge region, or two or more, for example, two isolated CDR or other epitope binding fragments of an antibody linked. An antibody fragment can also be incorporated into single domain antibodies, maxibodies, minibodies, nanobodies, intrabodies, diabodies, triabodies, tetrabodies, v-NAR and bis-scFv (see, for example, Hollinger and Hudson, Nature Biotechnology 23:1126-1136, 2005). Antibody fragments can also be grafted into scaffolds based on polypeptides such as a fibronectin type III (Fn3) (see U.S. Pat. No. 6,703,199, which describes fibronectin polypeptide minibodies).

The term “scFv” refers to a fusion protein comprising at least one antibody fragment comprising a variable region of a light chain and at least one antibody fragment comprising a variable region of a heavy chain, wherein the light and heavy chain variable regions are contiguously linked via a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, for example, with respect to the N-terminal and C-terminal ends of the polypeptide, the scFv may comprise VL-linker-VH or may comprise VH-linker-VL. In some embodiments, the scFv may comprise the structure of NH₂—V_{L}-linker-V_{H}—COOH or NH₂—V_{H}-linker-V_{L}-COOH.

The terms “complementarity determining region” or “CDR,” as used herein, refer to the sequences of amino acids within antibody variable regions which confer antigen specificity and binding affinity. For example, in general, there are three CDRs in each heavy chain variable region (for example, HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Kabat et al. (1991), “Sequences of Proteins of Immunological Interest,” 5th Ed. Public Health Service, National Institutes of Health, Bethesda, MD (“Kabat” numbering scheme), Al-Lazikani et al., (1997) JMB 273,927-948 (“Chothia” numbering scheme), or a combination thereof. In a combined Kabat and Chothia numbering scheme, in some embodiments, the CDRs correspond to the amino acid residues that are part of a Kabat CDR, a Chothia CDR, or both.

The portion of the CAR composition of the invention comprising an antibody or antibody fragment thereof may exist in a variety of forms, for example, where the antigen binding domain is expressed as part of a polypeptide chain including, for example, a single domain antibody fragment (sdAb), a single chain antibody (scFv), or for example, a human or humanized antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In some embodiments, the antigen binding domain of a CAR composition of the invention comprises an antibody fragment. In some embodiments, the CAR comprises an antibody fragment that comprises an scFv.

As used herein, the term “binding domain” or “antibody molecule” (also referred to herein as “anti-target binding domain”) refers to a protein, for example, an immunoglobulin chain or fragment thereof, comprising at least one immunoglobulin variable domain sequence. The term “binding domain” or “antibody molecule” encompasses antibodies and antibody fragments. In some embodiments, an antibody molecule is a multispecific antibody molecule, for example, it comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In some embodiments, a multispecific antibody molecule is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope.

The terms “bispecific antibody” and “bispecific antibodies” refer to molecules that combine the antigen binding sites of two antibodies within a single molecule. Thus, a bispecific antibody is able to bind two different antigens simultaneously or sequentially. Methods for making bispecific antibodies are well known in the art. Various formats for combining two antibodies are also known in the art. Forms of bispecific antibodies of the invention include, but are not limited to, a diabody, a single-chain diabody, Fab dimerization (Fab-Fab), Fab-scFv, and a tandem antibody, as known to those of skill in the art.

The term “antibody heavy chain,” refers to the larger of the two types of polypeptide chains present in antibody molecules in their naturally occurring conformations, and which normally determines the class to which the antibody belongs.

The term “antibody light chain,” refers to the smaller of the two types of polypeptide chains present in antibody molecules in their naturally occurring conformations. Kappa (K) and lambda (λ) light chains refer to the two major antibody light chain isotypes.

The term “recombinant antibody” refers to an antibody which is generated using recombinant DNA technology, such as, for example, an antibody expressed by a bacteriophage or yeast expression system. The term should also be construed to mean an antibody which has been generated by the synthesis of a DNA molecule encoding the antibody and which DNA molecule expresses an antibody protein, or an amino acid sequence specifying the antibody, wherein the DNA or amino acid sequence has been obtained using recombinant DNA or amino acid sequence technology which is available and well known in the art.

The term “antigen” or “Ag” refers to a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full length nucleotide sequence of a gene. It is readily apparent that the present invention includes, but is not limited to, the use of partial nucleotide sequences of more than one gene and that these nucleotide sequences are arranged in various combinations to encode polypeptides that elicit the desired immune response. Moreover, a skilled artisan will understand that an antigen need not be encoded by a “gene” at all. It is readily apparent that an antigen can be generated synthesized or can be derived from a biological sample, or might be macromolecule besides a polypeptide. Such a biological sample can include, but is not limited to a tissue sample, a tumor sample, a cell or a fluid with other biological components.

The terms “anti-tumor effect” and “anti-cancer effect” are used interchangeably and refer to a biological effect which can be manifested by various means, including but not limited to, for example, a decrease in tumor volume or cancer volume, a decrease in the number of tumor cells or cancer cells, a decrease in the number of metastases, an increase in life expectancy, a decrease in tumor cell proliferation or cancer cell proliferation, a decrease in tumor cell survival or cancer cell survival, or amelioration of various physiological symptoms associated with the cancerous condition. An “anti-tumor effect” or “anti-cancer effect” can also be manifested by the ability of the peptides, polynucleotides, cells and antibodies of the invention in prevention of the occurrence of tumor or cancer in the first place.

The term “autologous” refers to any material derived from the same individual to whom it is later to be re-introduced into the individual.

The term “allogeneic” refers to any material derived from a different animal of the same species as the individual to whom the material is introduced. Two or more individuals are said to be allogeneic to one another when the genes at one or more loci are not identical. In some embodiments, allogeneic material from individuals of the same species may be sufficiently unlike genetically to interact antigenically.

The term “xenogeneic” refers to a graft derived from an animal of a different species.

The term “apheresis” as used herein refers to the art-recognized extracorporeal process by which the blood of a donor or patient is removed from the donor or patient and passed through an apparatus that separates out selected particular constituent(s) and returns the remainder to the circulation of the donor or patient, for example, by retransfusion. Thus, in the context of “an apheresis sample” refers to a sample obtained using apheresis.

The term “cancer” refers to a disease characterized by the rapid and uncontrolled growth of aberrant cells. Cancer cells can spread locally or through the bloodstream and lymphatic system to other parts of the body. Examples of various cancers are described herein and include but are not limited to, breast cancer, prostate cancer, ovarian cancer, cervical cancer, skin cancer, pancreatic cancer, colorectal cancer, renal cancer, liver cancer, brain cancer, lymphoma, leukemia, lung cancer and the like. In some embodiments, cancers treated by the methods described herein include multiple myeloma, Hodgkin's lymphoma or non-Hodgkin's lymphoma.

The terms “tumor” and “cancer” are used interchangeably herein, for example, both terms encompass solid and liquid, for example, diffuse or circulating, tumors. As used herein, the term “cancer” or “tumor” includes premalignant, as well as malignant cancers and tumors.

“Derived from” as that term is used herein, indicates a relationship between a first and a second molecule. It generally refers to structural similarity between the first molecule and a second molecule and does not connotate or include a process or source limitation on a first molecule that is derived from a second molecule. For example, in the case of an intracellular signaling domain that is derived from a CD3zeta molecule, the intracellular signaling domain retains sufficient CD3zeta structure such that is has the required function, namely, the ability to generate a signal under the appropriate conditions. It does not connotate or include a limitation to a particular process of producing the intracellular signaling domain, for example, it does not mean that, to provide the intracellular signaling domain, one must start with a CD3zeta sequence and delete unwanted sequence, or impose mutations, to arrive at the intracellular signaling domain.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the invention by standard techniques known in the art, such as site-directed mutagenesis and PCR-mediated mutagenesis. Conservative substitutions are ones in which the amino acid residue is replaced with an amino acid residue having a similar side chain. Families of amino acid residues having similar side chains have been defined in the art. These families include amino acids with basic side chains (for example, lysine, arginine, histidine), acidic side chains (for example, aspartic acid, glutamic acid), uncharged polar side chains (for example, glycine, asparagine, glutamine, serine, threonine, tyrosine, cysteine, tryptophan), nonpolar side chains (for example, alanine, valine, leucine, isoleucine, proline, phenylalanine, methionine), beta-branched side chains (for example, threonine, valine, isoleucine) and aromatic side chains (for example, tyrosine, phenylalanine, tryptophan, histidine). Thus, one or more amino acid residues within a CAR of the invention can be replaced with other amino acid residues from the same side chain family and the altered CAR can be tested using the functional assays described herein.

The term “stimulation” in the context of stimulation by a stimulatory and/or costimulatory molecule refers to a response, for example, a primary or secondary response, induced by binding of a stimulatory molecule (for example, a TCR/CD3 complex) and/or a costimulatory molecule (for example, CD28 or 4-1BB) with its cognate ligand thereby mediating a signal transduction event, such as, but not limited to, signal transduction via the TCR/CD3 complex. Stimulation can mediate altered expression of certain molecules and/or reorganization of cytoskeletal structures, and the like.

The term “stimulatory molecule,” refers to a molecule expressed by a T cell that provides the primary cytoplasmic signaling sequence(s) that regulate primary activation of the TCR complex in a stimulatory way for at least some aspect of the T cell signaling pathway. In some embodiments, the ITAM-containing domain within the CAR recapitulates the signaling of the primary TCR independently of endogenous TCR complexes. In some embodiments, the primary signal is initiated by, for instance, binding of a TCR/CD3 complex with an MHC molecule loaded with peptide, and which leads to mediation of a T cell response, including, but not limited to, proliferation, activation, differentiation, and the like. A primary cytoplasmic signaling sequence (also referred to as a “primary signaling domain”) that acts in a stimulatory manner may contain a signaling motif which is known as immunoreceptor tyrosine-based activation motif or ITAM. Examples of an ITAM containing primary cytoplasmic signaling sequence that is of particular use in the invention includes, but is not limited to, those derived from TCR zeta, FcR gamma, FcR beta, CD3 gamma, CD3 delta, CD3 epsilon, CD5, CD22, CD79a, CD79b, CD278 (also known as “ICOS”), FcεRI and CD66d, DAP10 and DAP12. In a specific CAR of the invention, the intracellular signaling domain in any one or more CARS of the invention comprises an intracellular signaling sequence, for example, a primary signaling sequence of CD3-zeta. The term “antigen presenting cell” or “APC” refers to an immune system cell such as an accessory cell (for example, a B-cell, a dendritic cell, and the like) that displays a foreign antigen complexed with major histocompatibility complexes (MHC's) on its surface. T-cells may recognize these complexes using their T-cell receptors (TCRs). APCs process antigens and present them to T-cells.

An “intracellular signaling domain,” as the term is used herein, refers to an intracellular portion of a molecule. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

The intracellular signaling domain generates a signal that promotes an immune effector function of the CAR containing cell, for example, a CART cell. Examples of immune effector function, for example, in a CART cell, include cytolytic activity and helper activity, including the secretion of cytokines.

In some embodiments, the intracellular signaling domain can comprise a primary intracellular signaling domain. Exemplary primary intracellular signaling domains include those derived from the molecules responsible for primary stimulation, or antigen dependent simulation. In some embodiments, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3 zeta, FcR gamma, FcR beta, CD3 gamma, CD3 delta, CD3 epsilon, CD5, CD22, CD79a, CD79b, CD278 (also known as “ICOS”), FcεRI, CD66d, DAP10 and DAP12.

The term “zeta” or alternatively “zeta chain”, “CD3-zeta” or “TCR-zeta” refers to CD247. Swiss-Prot accession number P20963 provides exemplary human CD3 zeta amino acid sequences. A “zeta stimulatory domain” or alternatively a “CD3-zeta stimulatory domain” or a “TCR-zeta stimulatory domain” refers to a stimulatory domain of CD3-zeta or a variant thereof (for example, a molecule having mutations, for example, point mutations, fragments, insertions, or deletions). In some embodiments, the cytoplasmic domain of zeta comprises residues 52 through 164 of GenBank Acc. No. BAG36664.1 or a variant thereof (for example, a molecule having mutations, for example, point mutations, fragments, insertions, or deletions). In some embodiments, the “zeta stimulatory domain” or a “CD3-zeta stimulatory domain” is the sequence provided as SEQ ID NO: 9 or 10, or a variant thereof (for example, a molecule having mutations, for example, point mutations, fragments, insertions, or deletions).

The term “costimulatory molecule” refers to the cognate binding partner on a T cell that specifically binds with a costimulatory ligand, thereby mediating a costimulatory response by the T cell, such as, but not limited to, proliferation. Costimulatory molecules are cell surface molecules other than antigen receptors or their ligands that are required for an efficient immune response. Costimulatory molecules include, but are not limited to an MHC class I molecule, TNF receptor proteins, Immunoglobulin-like proteins, cytokine receptors, integrins, signaling lymphocytic activation molecules (SLAM proteins), activating NK cell receptors, BTLA, Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, and a ligand that specifically binds with CD83.

A costimulatory intracellular signaling domain refers to the intracellular portion of a costimulatory molecule.

The intracellular signaling domain can comprise the entire intracellular portion, or the entire native intracellular signaling domain, of the molecule from which it is derived, or a functional fragment thereof.

The term “4-1BB” refers to CD137 or Tumor necrosis factor receptor superfamily member 9. Swiss-Prot accession number P20963 provides exemplary human 4-1BB amino acid sequences. A “4-1BB costimulatory domain” refers to a costimulatory domain of 4-1BB, or a variant thereof (for example, a molecule having mutations, for example, point mutations, fragments, insertions, or deletions). In some embodiments, the “4-1BB costimulatory domain” is the sequence provided as SEQ ID NO: 7 or a variant thereof (for example, a molecule having mutations, for example, point mutations, fragments, insertions, or deletions).

“Immune effector cell,” as that term is used herein, refers to a cell that is involved in an immune response, for example, in the promotion of an immune effector response. Examples of immune effector cells include T cells, for example, alpha/beta T cells and gamma/delta T cells, B cells, natural killer (NK) cells, natural killer T (NKT) cells, mast cells, and myeloic-derived phagocytes.

“Immune effector function or immune effector response,” as that term is used herein, refers to function or response, for example, of an immune effector cell, that enhances or promotes an immune attack of a target cell. For example, an immune effector function or response refers a property of a T or NK cell that promotes killing or the inhibition of growth or proliferation, of a target cell. In the case of a T cell, primary stimulation and costimulation are examples of immune effector function or response.

The term “effector function” refers to a specialized function of a cell. Effector function of a T cell, for example, may be cytolytic activity or helper activity including the secretion of cytokines.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (for example, rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or a RNA may also include introns to the extent that the nucleotide sequence encoding the protein may in some version contain an intron(s).

The term “effective amount” or “therapeutically effective amount” are used interchangeably herein, and refer to an amount of a compound, formulation, material, or composition, as described herein effective to achieve a particular biological result.

The term “endogenous” refers to any material from or produced inside an organism, cell, tissue or system.

The term “exogenous” refers to any material introduced from or produced outside an organism, cell, tissue or system.

The term “expression” refers to the transcription and/or translation of a particular nucleotide sequence. In some embodiments, expression comprises translation of an mRNA introduced into a cell.

The term “transfer vector” refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “transfer vector” includes an autonomously replicating plasmid or a virus. The term should also be construed to further include non-plasmid and non-viral compounds which facilitate transfer of nucleic acid into cells, such as, for example, a polylysine compound, liposome, and the like. Examples of viral transfer vectors include, but are not limited to, adenoviral vectors, adeno-associated virus vectors, retroviral vectors, lentiviral vectors, and the like.

The term “expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, including cosmids, plasmids (for example, naked or contained in liposomes) and viruses (for example, lentiviruses, retroviruses, adenoviruses, and adeno-associated viruses) that incorporate the recombinant polynucleotide.

The term “lentivirus” refers to a genus of the Retroviridae family. Lentiviruses are unique among the retroviruses in being able to infect non-dividing cells; they can deliver a significant amount of genetic information into the DNA of the host cell, so they are one of the most efficient methods of a gene delivery vector. HIV, SIV, and FIV are all examples of lentiviruses.

The term “lentiviral vector” refers to a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include but are not limited to, for example, the LENTIVECTOR® gene delivery technology from Oxford BioMedica, the LENTIMAX™ vector system from Lentigen and the like. Nonclinical types of lentiviral vectors are also available and would be known to one skilled in the art.

The term “homologous” or “identity” refers to the subunit sequence identity between two polymeric molecules, for example, between two nucleic acid molecules, such as, two DNA molecules or two RNA molecules, or between two polypeptide molecules. When a subunit position in both of the two molecules is occupied by the same monomeric subunit; for example, if a position in each of two DNA molecules is occupied by adenine, then they are homologous or identical at that position. The homology between two sequences is a direct function of the number of matching or homologous positions; for example, if half (for example, five positions in a polymer ten subunits in length) of the positions in two sequences are homologous, the two sequences are 50% homologous; if 90% of the positions (for example, 9 of 10), are matched or homologous, the two sequences are 90% homologous.

“Humanized” forms of non-human (for example, murine) antibodies are chimeric immunoglobulins, immunoglobulin chains or fragments thereof (such as Fv, Fab, Fab′, F(ab′)2 or other antigen-binding subsequences of antibodies) which contain minimal sequence derived from non-human immunoglobulin. For the most part, humanized antibodies and antibody fragments thereof are human immunoglobulins (recipient antibody or antibody fragment) in which residues from a complementary-determining region (CDR) of the recipient are replaced by residues from a CDR of a non-human species (donor antibody) such as mouse, rat or rabbit having the desired specificity, affinity, and capacity. In some instances, Fv framework region (FR) residues of the human immunoglobulin are replaced by corresponding non-human residues. Furthermore, a humanized antibody/antibody fragment can comprise residues which are found neither in the recipient antibody nor in the imported CDR or framework sequences. These modifications can further refine and optimize antibody or antibody fragment performance. In general, the humanized antibody or antibody fragment thereof will comprise substantially all of at least one, and typically two, variable domains, in which all or substantially all of the CDR regions correspond to those of a non-human immunoglobulin and all or a significant portion of the FR regions are those of a human immunoglobulin sequence. The humanized antibody or antibody fragment can also comprise at least a portion of an immunoglobulin constant region (Fc), typically that of a human immunoglobulin. For further details, see Jones et al., Nature, 321: 522-525, 1986; Reichmann et al., Nature, 332: 323-329, 1988; Presta, Curr. Op. Struct. Biol., 2: 593-596, 1992.

“Fully human” refers to an immunoglobulin, such as an antibody or antibody fragment, where the whole molecule is of human origin or consists of an amino acid sequence identical to a human form of the antibody or immunoglobulin.

The term “isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

In the context of the present invention, the following abbreviations for the commonly occurring nucleic acid bases are used. “A” refers to adenosine, “C” refers to cytosine, “G” refers to guanosine, “T” refers to thymidine, and “U” refers to uridine.

The term “operably linked” or “transcriptional control” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Operably linked DNA sequences can be contiguous with each other and, for example, where necessary to join two protein coding regions, are in the same reading frame.

The term “parenteral” administration of an immunogenic composition includes, for example, subcutaneous (s.c.), intravenous (i.v.), intramuscular (i.m.), or intrasternal injection, intratumoral, or infusion techniques.

The term “nucleic acid,” “nucleic acid molecule,” “polynucleotide,” or “polynucleotide molecule” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. In some embodiments, a “nucleic acid,” “nucleic acid molecule,” “polynucleotide,” or “polynucleotide molecule” comprise a nucleotide/nucleoside derivative or analog. Unless otherwise indicated, a particular nucleic acid sequence also implicitly encompasses conservatively modified variants thereof (for example, degenerate codon substitutions, for example, conservative substitutions), alleles, orthologs, SNPs, and complementary sequences as well as the sequence explicitly indicated. Specifically, degenerate codon substitutions, for example, conservative substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Rossolini et al., Mol. Cell. Probes 8:91-98 (1994)).

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

The term “promoter” refers to a DNA sequence recognized by the synthetic machinery of the cell, or introduced synthetic machinery, required to initiate the specific transcription of a polynucleotide sequence.

The term “promoter/regulatory sequence” refers to a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

The term “constitutive” promoter refers to a nucleotide sequence which, when operably linked with a polynucleotide which encodes or specifies a gene product, causes the gene product to be produced in a cell under most or all physiological conditions of the cell.

The term “inducible” promoter refers to a nucleotide sequence which, when operably linked with a polynucleotide which encodes or specifies a gene product, causes the gene product to be produced in a cell substantially only when an inducer which corresponds to the promoter is present in the cell.

The term “tissue-specific” promoter refers to a nucleotide sequence which, when operably linked with a polynucleotide encodes or specified by a gene, causes the gene product to be produced in a cell substantially only if the cell is a cell of the tissue type corresponding to the promoter.

The terms “cancer associated antigen,” “tumor antigen,” “hyperproliferative disorder antigen,” and “antigen associated with a hyperproliferative disorder” interchangeably refer to antigens that are common to specific hyperproliferative disorders. In some embodiments, these terms refer to a molecule (typically a protein, carbohydrate or lipid) that is expressed on the surface of a cancer cell, either entirely or as a fragment (for example, MHC/peptide), and which is useful for the preferential targeting of a pharmacological agent to the cancer cell. In some embodiments, a tumor antigen is a marker expressed by both normal cells and cancer cells, for example, a lineage marker, for example, CD19 on B cells. In some embodiments, a tumor antigen is a cell surface molecule that is overexpressed in a cancer cell in comparison to a normal cell, for instance, 1-fold over expression, 2-fold overexpression, 3-fold overexpression or more in comparison to a normal cell. In some embodiments, a tumor antigen is a cell surface molecule that is inappropriately synthesized in the cancer cell, for instance, a molecule that contains deletions, additions or mutations in comparison to the molecule expressed on a normal cell. In some embodiments, a tumor antigen will be expressed exclusively on the cell surface of a cancer cell, entirely or as a fragment (for example, MHC/peptide), and not synthesized or expressed on the surface of a normal cell. In some embodiments, the hyperproliferative disorder antigens of the present invention are derived from, cancers including but not limited to primary or metastatic melanoma, thymoma, lymphoma, sarcoma, lung cancer, liver cancer, non-Hodgkin lymphoma, Hodgkin lymphoma, leukemias, uterine cancer, cervical cancer, bladder cancer, kidney cancer and adenocarcinomas such as breast cancer, prostate cancer (for example, castrate-resistant or therapy-resistant prostate cancer, or metastatic prostate cancer), ovarian cancer, pancreatic cancer, and the like, or a plasma cell proliferative disorder, for example, asymptomatic myeloma (smoldering multiple myeloma or indolent myeloma), monoclonal gammopathy of undetermined significance (MGUS), Waldenstrom's macroglobulinemia, plasmacytomas (for example, plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome). In some embodiments, the CARs of the present invention include CARs comprising an antigen binding domain (for example, antibody or antibody fragment) that binds to a MHC presented peptide. Normally, peptides derived from endogenous proteins fill the pockets of Major histocompatibility complex (MHC) class I molecules and are recognized by T cell receptors (TCRs) on CD8+T lymphocytes. The MHC class I complexes are constitutively expressed by all nucleated cells. In cancer, virus-specific and/or tumor-specific peptide/MHC complexes represent a unique class of cell surface targets for immunotherapy. TCR-like antibodies targeting peptides derived from viral or tumor antigens in the context of human leukocyte antigen (HLA)-A1 or HLA-A2 have been described (see, for example, Sastry et al., J Virol. 2011 85(5):1935-1942; Sergeeva et al., Blood, 2011 117(16):4262-4272; Verma et al., J Immunol 2010 184(4):2156-2165; Willemsen et al., Gene Ther 2001 8(21):1601-1608; Dao et al., Sci Transl Med 2013 5(176):176ra33; Tassev et al., Cancer Gene Ther 2012 19(2):84-100). For example, TCR-like antibody can be identified from screening a library, such as a human scFv phage displayed library.

The term “tumor-supporting antigen” or “cancer-supporting antigen” interchangeably refer to a molecule (typically a protein, carbohydrate or lipid) that is expressed on the surface of a cell that is, itself, not cancerous, but supports the cancer cells, for example, by promoting their growth or survival for example, resistance to immune cells. Exemplary cells of this type include stromal cells and myeloid-derived suppressor cells (MDSCs). The tumor-supporting antigen itself need not play a role in supporting the tumor cells so long as the antigen is present on a cell that supports cancer cells.

The term “flexible polypeptide linker” or “linker” as used in the context of an scFv refers to a peptide linker that consists of amino acids such as glycine and/or serine residues used alone or in combination, to link variable heavy and variable light chain regions together. In some embodiments, the flexible polypeptide linker is a Gly/Ser linker and comprises the amino acid sequence (Gly-Gly-Gly-Ser)n, where n is a positive integer equal to or greater than 1 (SEQ ID NO: 41). For example, n=1, n=2, n=3. n=4, n=5 and n=6, n=7, n=8, n=9 and n=10 In some embodiments, the flexible polypeptide linkers include, but are not limited to, (Gly4 Ser)4 (SEQ ID NO: 27) or (Gly4 Ser)3 (SEQ ID NO: 28). In some embodiments, the linkers include multiple repeats of (Gly2Ser), (GlySer) or (Gly3Ser) (SEQ ID NO: 29). Also included within the scope of the invention are linkers described in WO2012/138475, incorporated herein by reference.

As used herein, a 5′ cap (also termed an RNA cap, an RNA 7-methylguanosine cap or an RNA m7G cap) is a modified guanine nucleotide that has been added to the “front” or 5′ end of a eukaryotic messenger RNA shortly after the start of transcription. The 5′ cap consists of a terminal group which is linked to the first transcribed nucleotide. Its presence is critical for recognition by the ribosome and protection from RNases. Cap addition is coupled to transcription, and occurs co-transcriptionally, such that each influences the other. Shortly after the start of transcription, the 5′ end of the mRNA being synthesized is bound by a cap-synthesizing complex associated with RNA polymerase. This enzymatic complex catalyzes the chemical reactions that are required for mRNA capping. Synthesis proceeds as a multi-step biochemical reaction. The capping moiety can be modified to modulate functionality of mRNA such as its stability or efficiency of translation.

As used herein, “in vitro transcribed RNA” refers to RNA that has been synthesized in vitro. In some embodiments the RNA is mRNA. Generally, the in vitro transcribed RNA is generated from an in vitro transcription vector. The in vitro transcription vector comprises a template that is used to generate the in vitro transcribed RNA.

As used herein, a “poly(A)” is a series of adenosines attached by polyadenylation to the mRNA. In some embodiments of a construct for transient expression, the poly(A) is between 50 and 5000 (SEQ ID NO: 30). In some embodiments the poly(A) is greater than 64. In some embodiments the poly(A) is greater than 100. In some embodiments the poly(A) is greater than 300. In some embodiments the poly(A) is greater than 400. poly(A) sequences can be modified chemically or enzymatically to modulate mRNA functionality such as localization, stability or efficiency of translation.

As used herein, “polyadenylation” refers to the covalent linkage of a polyadenylyl moiety, or its modified variant, to a messenger RNA molecule. In eukaryotic organisms, most messenger RNA (mRNA) molecules are polyadenylated at the 3′ end. The 3′ poly(A) tail is a long sequence of adenine nucleotides (often several hundred) added to the pre-mRNA through the action of an enzyme, polyadenylate polymerase. In higher eukaryotes, the poly(A) tail is added onto transcripts that contain a specific sequence, the polyadenylation signal. The poly(A) tail and the protein bound to it aid in protecting mRNA from degradation by exonucleases. Polyadenylation is also important for transcription termination, export of the mRNA from the nucleus, and translation. Polyadenylation occurs in the nucleus immediately after transcription of DNA into RNA, but additionally can also occur later in the cytoplasm. After transcription has been terminated, the mRNA chain is cleaved through the action of an endonuclease complex associated with RNA polymerase. The cleavage site is usually characterized by the presence of the base sequence AAUAAA near the cleavage site. After the mRNA has been cleaved, adenosine residues are added to the free 3′ end at the cleavage site.

As used herein, “transient” refers to expression of a non-integrated transgene for a period of hours, days or weeks, wherein the period of time of expression is less than the period of time for expression of the gene if integrated into the genome or contained within a stable plasmid replicon in the host cell.

As used herein, the terms “treat”, “treatment” and “treating” refer to the reduction or amelioration of the progression, severity and/or duration of a proliferative disorder, or the amelioration of one or more symptoms (preferably, one or more discernible symptoms) of a proliferative disorder resulting from the administration of one or more therapies (for example, one or more therapeutic agents such as a CAR of the invention). In specific embodiments, the terms “treat”, “treatment” and “treating” refer to the amelioration of at least one measurable physical parameter of a proliferative disorder, such as growth of a tumor, not necessarily discernible by the patient. In other embodiments the terms “treat”, “treatment” and “treating”-refer to the inhibition of the progression of a proliferative disorder, either physically by, for example, stabilization of a discernible symptom, physiologically by, for example, stabilization of a physical parameter, or both. In other embodiments the terms “treat”, “treatment” and “treating” refer to the reduction or stabilization of tumor size or cancerous cell count.

The term “signal transduction pathway” refers to the biochemical relationship between a variety of signal transduction molecules that play a role in the transmission of a signal from one portion of a cell to another portion of a cell. The phrase “cell surface receptor” includes molecules and complexes of molecules capable of receiving a signal and transmitting signal across the membrane of a cell.

The term “subject” is intended to include living organisms in which an immune response can be elicited (for example, mammals, for example, human).

The term, a “substantially purified” cell refers to a cell that is essentially free of other cell types. A substantially purified cell also refers to a cell which has been separated from other cell types with which it is normally associated in its naturally occurring state. In some instances, a population of substantially purified cells refers to a homogenous population of cells. In other instances, this term refers simply to cell that have been separated from the cells with which they are naturally associated in their natural state. In some embodiments, the cells are cultured in vitro. In some embodiments, the cells are not cultured in vitro.

The term “therapeutic” as used herein means a treatment. A therapeutic effect is obtained by reduction, suppression, remission, or eradication of a disease state.

The term “prophylaxis” as used herein means the prevention of or protective treatment for a disease or disease state.

The term “transfected” or “transformed” or “transduced” refers to a process by which exogenous nucleic acid is transferred or introduced into the host cell. A “transfected” or “transformed” or “transduced” cell is one which has been transfected, transformed or transduced with exogenous nucleic acid. The cell includes the primary subject cell and its progeny.

The term “specifically binds,” refers to an antibody, or a ligand, which recognizes and binds with a cognate binding partner (for example, a stimulatory and/or costimulatory molecule present on a T cell) protein present in a sample, but which antibody or ligand does not substantially recognize or bind other molecules in the sample.

“Regulatable chimeric antigen receptor (RCAR),” as used herein, refers to a set of polypeptides, typically two in the simplest embodiments, which when in an immune effector cell, provides the cell with specificity for a target cell, typically a cancer cell, and with intracellular signal generation. In some embodiments, an RCAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined herein in the context of a CAR molecule. In some embodiments, the set of polypeptides in the RCAR are not contiguous with each other, for example, are in different polypeptide chains. In some embodiments, the RCAR includes a dimerization switch that, upon the presence of a dimerization molecule, can couple the polypeptides to one another, for example, can couple an antigen binding domain to an intracellular signaling domain. In some embodiments, the RCAR is expressed in a cell (for example, an immune effector cell) as described herein, for example, an RCAR-expressing cell (also referred to herein as “RCARX cell”). In some embodiments the RCARX cell is a T cell and is referred to as a RCART cell. In some embodiments the RCARX cell is an NK cell, and is referred to as a RCARN cell. The RCAR can provide the RCAR-expressing cell with specificity for a target cell, typically a cancer cell, and with regulatable intracellular signal generation or proliferation, which can optimize an immune effector property of the RCAR-expressing cell. In embodiments, an RCAR cell relies at least in part, on an antigen binding domain to provide specificity to a target cell that comprises the antigen bound by the antigen binding domain.

“Membrane anchor” or “membrane tethering domain”, as that term is used herein, refers to a polypeptide or moiety, for example, a myristoyl group, sufficient to anchor an extracellular or intracellular domain to the plasma membrane.

“Switch domain,” as that term is used herein, for example, when referring to an RCAR, refers to an entity, typically a polypeptide-based entity, that, in the presence of a dimerization molecule, associates with another switch domain. The association results in a functional coupling of a first entity linked to, for example, fused to, a first switch domain, and a second entity linked to, for example, fused to, a second switch domain. A first and second switch domain are collectively referred to as a dimerization switch. In embodiments, the first and second switch domains are the same as one another, for example, they are polypeptides having the same primary amino acid sequence and are referred to collectively as a homodimerization switch. In embodiments, the first and second switch domains are different from one another, for example, they are polypeptides having different primary amino acid sequences, and are referred to collectively as a heterodimerization switch. In embodiments, the switch is intracellular. In embodiments, the switch is extracellular. In embodiments, the switch domain is a polypeptide-based entity, for example, FKBP or FRB-based, and the dimerization molecule is small molecule, for example, a rapalogue. In embodiments, the switch domain is a polypeptide-based entity, for example, an scFv that binds a myc peptide, and the dimerization molecule is a polypeptide, a fragment thereof, or a multimer of a polypeptide, for example, a myc ligand or multimers of a myc ligand that bind to one or more myc scFvs. In embodiments, the switch domain is a polypeptide-based entity, for example, myc receptor, and the dimerization molecule is an antibody or fragments thereof, for example, myc antibody.

“Dimerization molecule,” as that term is used herein, for example, when referring to an RCAR, refers to a molecule that promotes the association of a first switch domain with a second switch domain. In embodiments, the dimerization molecule does not naturally occur in the subject or does not occur in concentrations that would result in significant dimerization. In embodiments, the dimerization molecule is a small molecule, for example, rapamycin or a rapalogue, for example, RAD001.

The term “low, immune enhancing, dose” when used in conjunction with an mTOR inhibitor, for example, an allosteric mTOR inhibitor, for example, RAD001 or rapamycin, or a catalytic mTOR inhibitor, refers to a dose of mTOR inhibitor that partially, but not fully, inhibits mTOR activity, for example, as measured by the inhibition of P70 S6 kinase activity. Methods for evaluating mTOR activity, for example, by inhibition of P70 S6 kinase, are discussed herein. The dose is insufficient to result in complete immune suppression but is sufficient to enhance the immune response. In some embodiments, the low, immune enhancing, dose of mTOR inhibitor results in a decrease in the number of PD-1 positive T cells and/or an increase in the number of PD-1 negative T cells, or an increase in the ratio of PD-1 negative T cells/PD-1 positive T cells. In some embodiments, the low, immune enhancing, dose of mTOR inhibitor results in an increase in the number of naïve T cells. In some embodiments, the low, immune enhancing, dose of mTOR inhibitor results in one or more of the following:
an increase in the expression of one or more of the following markers: CD62L^{high}, CD127^{high}, CD27⁺, and BCL2, for example, on memory T cells, for example, memory T cell precursors;
a decrease in the expression of KLRG1, for example, on memory T cells, for example, memory T cell precursors; and
an increase in the number of memory T cell precursors, for example, cells with any one or combination of the following characteristics: increased CD62L^{high} increased CD127^{high} increased CD27⁺, decreased KLRG1, and increased BCL2;
wherein any of the changes described above occurs, for example, at least transiently, for example, as compared to a non-treated subject.

“Refractory” as used herein refers to a disease, for example, cancer, that does not respond to a treatment. In embodiments, a refractory cancer can be resistant to a treatment before or at the beginning of the treatment. In other embodiments, the refractory cancer can become resistant during a treatment. A refractory cancer is also called a resistant cancer.

“Relapsed” or “relapse” as used herein refers to the return or reappearance of a disease (for example, cancer) or the signs and symptoms of a disease such as cancer after a period of improvement or responsiveness, for example, after prior treatment of a therapy, for example, cancer therapy. The initial period of responsiveness may involve the level of cancer cells falling below a certain threshold, for example, below 20%, 1%, 10%, 5%, 4%, 3%, 2%, or 1%. The reappearance may involve the level of cancer cells rising above a certain threshold, for example, above 20%, 1%, 10%, 5%, 4%, 3%, 2%, or 1%. For example, for example, in the context of B-ALL, the reappearance may involve, for example, a reappearance of blasts in the blood, bone marrow (>5%), or any extramedullary site, after a complete response. A complete response, in this context, may involve <5% BM blast. More generally, in some embodiments, a response (for example, complete response or partial response) can involve the absence of detectable MRD (minimal residual disease). In some embodiments, the initial period of responsiveness lasts at least 1, 2, 3, 4, 5, or 6 days; at least 1, 2, 3, or 4 weeks; at least 1, 2, 3, 4, 6, 8, 10, or 12 months; or at least 1, 2, 3, 4, or 5 years.

Ranges: throughout this disclosure, various embodiments of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. As another example, a range such as 95-99% identity, includes something with 95%, 96%, 97%, 98%, or 99% identity, and includes subranges such as 96-99%, 96-98%, 96-97%, 97-99%, 97-98%, and 98-99% identity. This applies regardless of the breadth of the range.

A “gene editing system” as the term is used herein, refers to a system, for example, one or more molecules, that direct and effect an alteration, for example, a deletion, of one or more nucleic acids at or near a site of genomic DNA targeted by said system. Gene editing systems are known in the art and are described more fully below.

Administered “in combination”, as used herein, means that two (or more) different treatments are delivered to the subject during the course of the subject's affliction with the disorder, for example, the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, for example, an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The term “depletion” or “depleting”, as used interchangeably herein, refers to the decrease or reduction of the level or amount of a cell, a protein, or macromolecule in a sample after a process, for example, a selection step, for example, a negative selection, is performed. The depletion can be a complete or partial depletion of the cell, protein, or macromolecule. In some embodiments, the depletion is at least a 1%, 2%, 5%, 10%, 15%, 20%, 25%, 30%, 35%, 40%, 45%, 50%, 55%, 60%, 65%, 70%, 75%, 80%, 85%, 90%, 95%, or 99% decrease or reduction of the level or amount of a cell, a protein, or macromolecule, as compared to the level or amount of the cell, protein or macromolecule in the sample before the process was performed.

As used herein, a “naïve T cell” refers to a T cell that is antigen-inexperienced. In some embodiments, an antigen-inexperienced T cell has encountered its cognate antigen in the thymus but not in the periphery. In some embodiments, naïve T cells are precursors of memory cells. In some embodiments, naïve T cells express both CD45RA and CCR7, but do not express CD45RO. In some embodiments, naïve T cells may be characterized by expression of CD62L, CD27, CCR7, CD45RA, CD28, and CD127, and the absence of CD95 or CD45RO isoform. In some embodiments, naïve T cells express CD62L, IL-7 receptor-α, IL-6 receptor, and CD132, but do not express CD25, CD44, CD69, or CD45RO. In some embodiments, naïve T cells express CD45RA, CCR7, and CD62L and do not express CD95 or IL-2 receptor β. In some embodiments, surface expression levels of markers are assessed using flow cytometry.

The term “central memory T cells” refers to a subset of T cells that in humans are CD45RO positive and express CCR7. In some embodiments, central memory T cells express CD95. In some embodiments, central memory T cells express IL-2R, IL-7R and/or IL-15R. In some embodiments, central memory T cells express CD45RO, CD95, IL-2 receptor R, CCR7, and CD62L. In some embodiments, surface expression levels of markers are assessed using flow cytometry.

The term “stem memory T cells,” “stem cell memory T cells,” “stem cell-like memory T cells,” “memory stem T cells,” “T memory stem cells,” “T stem cell memory cells” or “TSCM cells” refers to a subset of memory T cells with stem cell-like ability, for example, the ability to self-renew and/or the multipotent capacity to reconstitute memory and/or effector T cell subsets. In some embodiments, stem memory T cells express CD45RA, CD95, IL-2 receptor R, CCR7, and CD62L. In some embodiments, surface expression levels of markers are assessed using flow cytometry. In some embodiments, exemplary stem memory T cells are disclosed in Gattinoni et al., Nat Med. 2017 Jan. 6; 23(1): 18-27, herein incorporated by reference in its entirety.

For clarity purposes, unless otherwise noted, classifying a cell or a population of cells as “not expressing,” or having an “absence of” or being “negative for” a particular marker may not necessarily mean an absolute absence of the marker. The skilled artisan can readily compare the cell against a positive and/or a negative control, and/or set a predetermined threshold, and classify the cell or population of cells as not expressing or being negative for the marker when the cell has an expression level below the predetermined threshold or a population of cells has an overall expression level below the predetermined threshold using conventional detection methods, e.g., using flow cytometry, for example, as described in the Examples herein.

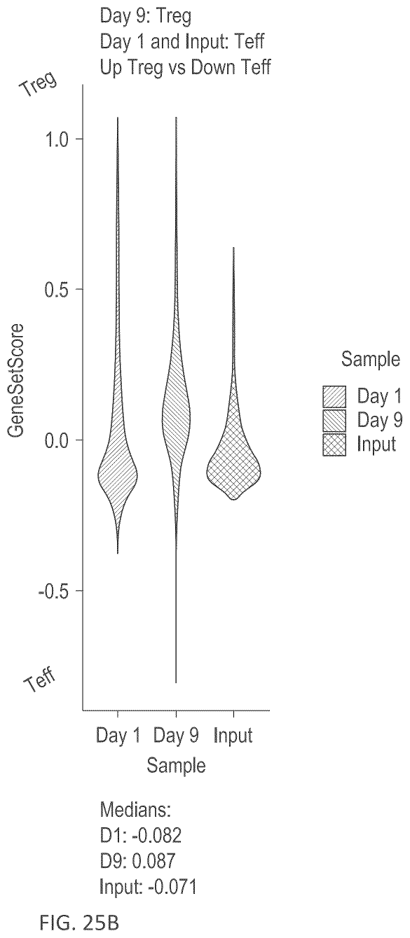

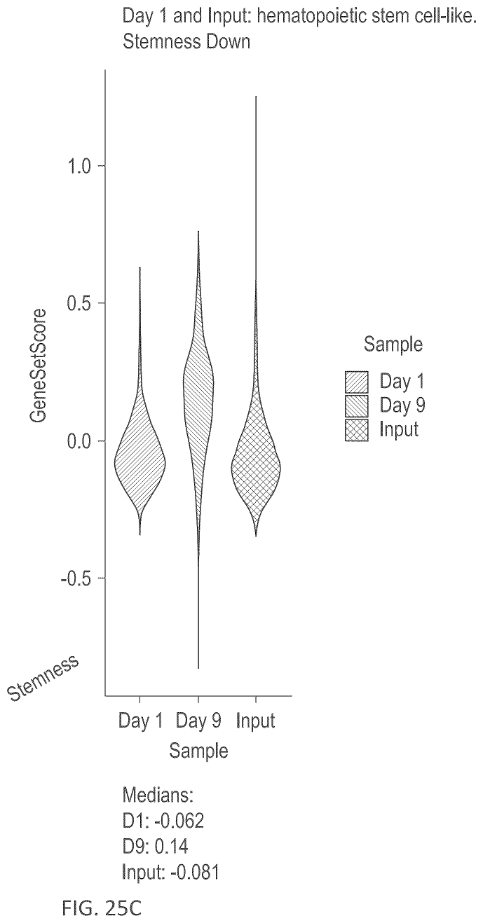

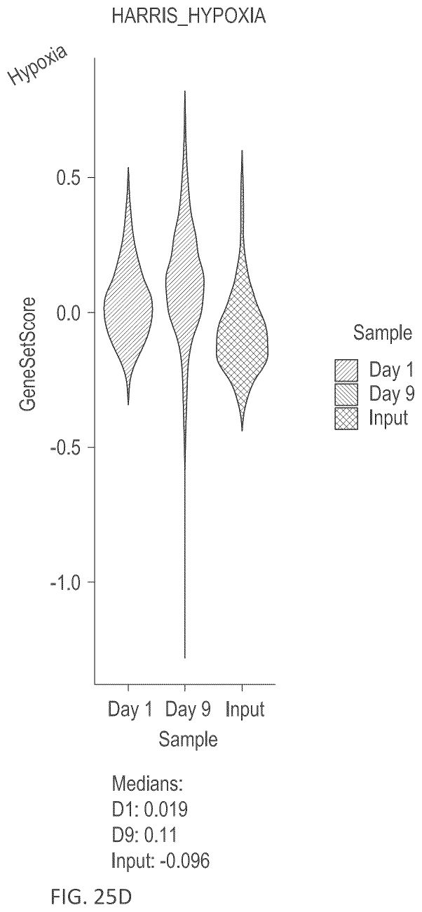

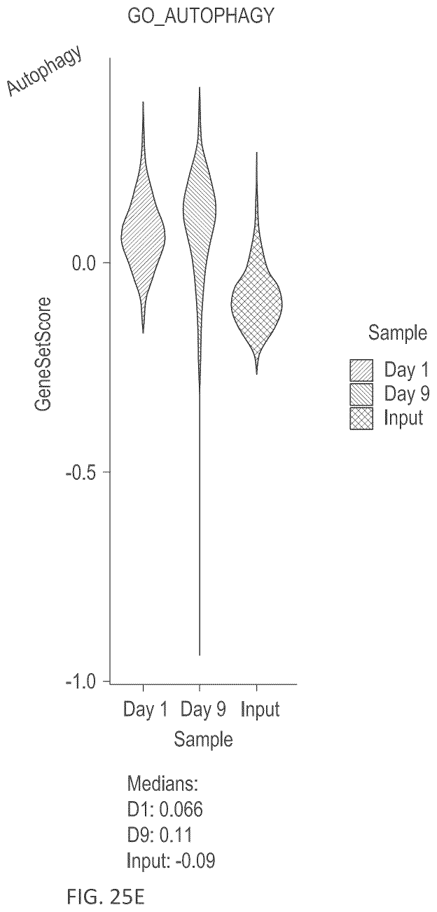

As used herein, the term “GeneSetScore (Up TEM vs. Down TSCM)” of a cell refers to a score that reflects the degree at which the cell shows an effector memory T cell (TEM) phenotype vs. a stem cell memory T cell (TSCM) phenotype. A higher GeneSetScore (Up TEM vs. Down TSCM) indicates an increasing TEM phenotype, whereas a lower GeneSetScore (Up TEM vs. Down TSCM) indicates an increasing TSCM phenotype. In some embodiments, the GeneSetScore (Up TEM vs. Down TSCM) is determined by measuring the expression of one or more genes that are up-regulated in TEM cells and/or down-regulated in TSCM cells, for example, one or more genes selected from the group consisting of MXRA7, CLIC1, NAT13, TBC1D2B, GLCCI1, DUSP10, APOBEC3D, CACNB3, ANXA2P2, TPRG1, EOMES, MATK, ARHGAP10, ADAM8, MAN1A1, SLFN12L, SH2D2A, EIF2C4, CD58, MYO1F, RAB27B, ERN1, NPC1, NBEAL2, APOBEC3G, SYTL2, SLC4A4, PIK3AP1, PTGDR, MAF, PLEKHA5, ADRB2, PLXND1, GNAO1, THBS1, PPP2R2B, CYTH3, KLRF1, FLJ16686, AUTS2, PTPRM, GNLY, and GFPT2. In some embodiments, the GeneSetScore (Up TEM vs. Down TSCM) is determined for each cell using RNA-seq, for example, single-cell RNA-seq (scRNA-seq), for example, as exemplified in Example 7 with respect to FIG. 25A. In some embodiments, the GeneSetScore (Up TEM vs. Down TSCM) is calculated by taking the mean log normalized gene expression value of all of the genes in the gene set.

As used herein, the term “GeneSetScore (Up Treg vs. Down Teff)” of a cell refers to a score that reflects the degree at which the cell shows a regulatory T cell (Treg) phenotype vs. an effector T cell (Teff) phenotype. A higher GeneSetScore (Up Treg vs. Down Teff) indicates an increasing Treg phenotype, whereas a lower GeneSetScore (Up Treg vs. Down Teff) indicates an increasing Teff phenotype. In some embodiments, the GeneSetScore (Up Treg vs. Down Teff) is determined by measuring the expression of one or more genes that are up-regulated in Treg cells and/or down-regulated in Teff cells, for example, one or more genes selected from the group consisting of C12orf75, SELPLG, SWAP70, RGS1, PRR11, SPATS2L, SPATS2L, TSHR, C14orf145, CASP8, SYT11, ACTN4, ANXA5, GLRX, HLA-DMB, PMCH, RAB11FIP1, IL32, FAM160B1, SHMT2, FRMD4B, CCR3, TNFRSF13B, NTNG2, CLDND1, BARD1, FCER1G, TYMS, ATP1B1, GJB6, FGL2, TK1, SLC2A8, CDKN2A, SKAP2, GPR55, CDCA7, S100A4, GDPD5, PMAIP1, ACOT9, CEP55, SGMS1, ADPRH, AKAP2, HDAC9, IKZF4, CARD17, VAV3, OBFC2A, ITGB1, CIITA, SETD7, HLA-DMA, CCR10, KIAA0101, SLC14A1, PTTG3P, DUSP10, FAM164A, PYHIN1, MYO1F, SLC1A4, MYBL2, PTTG1, RRM2, TP53INP1, CCR5, ST8SIA6, TOX, BFSP2, ITPRIPL1, NCAPH, HLA-DPB2, SYT4, NINJ2, FAM46C, CCR4, GBP5, C15orf53, LMCD1, MKI67, NUSAP1, PDE4A, E2F2, CD58, ARHGEF12, LOC100188949, FAS, HLA-DPB1, SELP, WEE1, HLA-DPA1, FCRL1, ICA1, CNTNAP1, OAS1, METTL7A, CCR6, HLA-DRB4, ANXA2P3, STAM, HLA-DQB2, LGALS1, ANXA2, PI16, DUSP4, LAYN, ANXA2P2, PTPLA, ANXA2P1, ZNF365, LAIR2, LOC541471, RASGRP4, BCAS1, UTS2, MIAT, PRDM1, SEMA3G, FAM129A, HPGD, NCF4, LGALS3, CEACAM4, JAKMIP1, TIGIT, HLA-DRA, IKZF2, HLA-DRB1, FANK1, RTKN2, TRIB1, FCRL3, and FOXP3. In some embodiments, the GeneSetScore (Up Treg vs. Down Teff) is determined using RNA-seq, for example, single-cell RNA-seq (scRNA-seq), for example, as exemplified in Example 7 with respect to FIG. 25B. In some embodiments, the GeneSetScore (Up Treg vs. Down Teff) is calculated by taking the mean log normalized gene expression value of all of the genes in the gene set.

As used herein, the term “GeneSetScore (Down stemness)” of a cell refers to a score that reflects the degree at which the cell shows a stemness phenotype. A lower GeneSetScore (Down stemness) indicates an increasing stemness phenotype. In some embodiments, the GeneSetScore (Down stemness) is determined by measuring the expression of one or more genes that are upregulated in a differentiating stem cell vs downregulated in a hematopoietic stem cell, for example, one or more genes selected from the group consisting of ACE, BATF, CDK6, CHD2, ERCC2, HOXB4, MEOX1, SFRP1, SP7, SRF, TAL1, and XRCC5. In some embodiments, the GeneSetScore (Down stemness) is determined using RNA-seq, for example, single-cell RNA-seq (scRNA-seq), for example, as exemplified in Example 7 with respect to FIG. 25C. In some embodiments, the GeneSetScore (Down stemness) is calculated by taking the mean log normalized gene expression value of all of the genes in the gene set.

As used herein, the term “GeneSetScore (Up hypoxia)” of a cell refers to a score that reflects the degree at which the cell shows a hypoxia phenotype. A higher GeneSetScore (Up hypoxia) indicates an increasing hypoxia phenotype. In some embodiments, the GeneSetScore (Up hypoxia) is determined by measuring the expression of one or more genes that are up-regulated in cells undergoing hypoxia, for example, one or more genes selected from the group consisting of ABCB1, ACAT1, ADM, ADORA2B, AK2, AK3, ALDH1A1, ALDH1A3, ALDOA, ALDOC, ANGPT2, ANGPTL4, ANXA1, ANXA2, ANXA5, ARHGAP5, ARSE, ART1, BACE2, BATF3, BCL2L1, BCL2L2, BHLHE40, BHLHE41, BIK, BIRC2, BNIP3, BNIP3L, BPI, BTG1, C11orf2, C7orf68, CA12, CA9, CALD1, CCNG2, CCT6A, CD99, CDK1, CDKN1A, CDKN1B, CITED2, CLK1, CNOT7, COL4A5, COL5A1, COL5A2, COL5A3, CP, CTSD, CXCR4, D4S234E, DDIT3, DDIT4, 1-Dec, DKC1, DR1, EDN1, EDN2, EFNA1, EGF, EGR1, EIF4A3, ELF3, ELL2, ENG, ENO1, ENO3, ENPEP, EPO, ERRFI1, ETS1, F3, FABP5, FGF3, FKBP4, FLT1, FN1, FOS, FTL, GAPDH, GBE1, GLRX, GPI, GPRC5A, HAP1, HBP1, HDAC1, HDAC9, HERC3, HERPUD1, HGF, HIF1A, HK1, HK2, HLA-DQB1, HMOX1, HMOX2, HSPA5, HSPD1, HSPH1, HYOU1, ICAM1, ID2, IFI27, IGF2, IGFBP1, IGFBP2, IGFBP3, IGFBP5, IL6, IL8, INSIG1, IRF6, ITGA5, JUN, KDR, KRT14, KRT18, KRT19, LDHA, LDHB, LEP, LGALS1, LONP1, LOX, LRP1, MAP4, MET, MIF, MMP13, MMP2, MMP7, MPI, MT1L, MTL3P, MUC1, MXIl, NDRG1, NFIL3, NFKB1, NFKB2, NOS1, NOS2, NOS2P1, NOS2P2, NOS3, NR3C1, NR4A1, NT5E, ODC1, P4HA1, P4HA2, PAICS, PDGFB, PDK3, PFKFB1, PFKFB3, PFKFB4, PFKL, PGAM1, PGF, PGK1, PGK2, PGM1, PIM1, PIM2, PKM2, PLAU, PLAUR, PLIN2, PLOD2, PNN, PNP, POLM, PPARA, PPAT, PROK1, PSMA3, PSMD9, PTGS1, PTGS2, QSOX1, RBPJ, RELA, RIOK3, RNASEL, RPL36A, RRP9, SAT1, SERPINB2, SERPINE1, SGSM2, SIAH2, SIN3A, SIRPA, SLC16A1, SLC16A2, SLC20A1, SLC2A1, SLC2A3, SLC3A2, SLC6A10P, SLC6A16, SLC6A6, SLC6A8, SORL1, SPP1, SRSF6, SSSCA1, STC2, STRA13, SYT7, TBPL1, TCEAL1, TEK, TF, TFF3, TFRC, TGFA, TGFB1, TGFB3, TGFBI, TGM2, TH, THBS1, THBS2, TIMM17A, TNFAIP3, TP53, TPBG, TPD52, TPI1, TXN, TXNIP, UMPS, VEGFA, VEGFB, VEGFC, VIM, VPS11, and XRCC6. In some embodiments, the GeneSetScore (Up hypoxia) is determined using RNA-seq, for example, single-cell RNA-seq (scRNA-seq), for example, as exemplified in Example 7 with respect to FIG. 25D. In some embodiments, the GeneSetScore (Up hypoxia) is calculated by taking the mean log normalized gene expression value of all of the genes in the gene set.

As used herein, the term “GeneSetScore (Up autophagy)” of a cell refers to a score that reflects the degree at which the cell shows an autophagy phenotype. A higher GeneSetScore (Up autophagy) indicates an increasing autophagy phenotype. In some embodiments, the GeneSetScore (Up autophagy) is determined by measuring the expression of one or more genes that are up-regulated in cells undergoing autophagy, for example, one or more genes selected from the group consisting of ABL1, ACBD5, ACIN1, ACTRT1, ADAMTS7, AKR1E2, ALKBH5, ALPK1, AMBRA1, ANXA5, ANXA7, ARSB, ASB2, ATG10, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATP13A2, ATP1B1, ATPAF1-AS1, ATPIF1, BECN1, BECN1P1, BLOC1S1, BMP2KL, BNIP1, BNIP3, BOC, C11orf2, C11orf4l, C12orf44, C12orf5, C14orf133, C1orf210, C5, C6orf106, C7orf59, C7orf68, C8orf59, C9orf72, CA7, CALCB, CALCOCO2, CAPS, CCDC36, CD163L1, CD93, CDC37, CDKN2A, CHAF1B, CHMP2A, CHMP2B, CHMP3, CHMP4A, CHMP4B, CHMP4C, CHMP6, CHST3, CISD2, CLDN7, CLEC16A, CLN3, CLVS1, COX8A, CPA3, CRNKL1, CSPG5, CTSA, CTSB, CTSD, CXCR7, DAP, DKKL1, DNAAF2, DPF3, DRAM1, DRAM2, DYNLL1, DYNLL2, DZANK1, E124, EIF2S1, EPG5, EPM2A, FABP1, FAM125A, FAM131B, FAM134B, FAM13B, FAM176A, FAM176B, FAM48A, FANCC, FANCF, FANCL, FBXO7, FCGR3B, FGF14, FGF7, FGFBP1, FIS1, FNBP1L, FOXO1, FUNDC1, FUNDC2, FXR2, GABARAP, GABARAPL1, GABARAPL2, GABARAPL3, GABRA5, GDF5, GMIP, HAP1, HAPLN1, HBXIP, HCAR1, HDAC6, HGS, HIST1H3A, HIST1H3B, HIST1H3C, HIST1H3D, HIST1H3E, HIST1H3F, HIST1H3G, HIST1H3H, HIST1H3I, HIST1H3J, HK2, HMGB1, HPR, HSF2BP, HSP90AA1, HSPA8, IFI16, IPPK, IRGM, IST1, ITGB4, ITPKC, KCNK3, KCNQ1, KIAA0226, KIAA1324, KRCC1, KRT15, KRT73, LAMP1, LAMP2, LAMTOR1, LAMTOR2, LAMTOR3, LARP1B, LENG9, LGALS8, LIX1, LIX1L, LMCD1, LRRK2, LRSAM1, LSM4, MAP1A, MAP1LC3A, MAP1LC3B, MAP1LC3B2, MAP1LC3C, MAP1S, MAP2K1, MAP3K12, MARK2, MBD5, MDH1, MEX3C, MFN1, MFN2, MLST8, MRPS10, MRPS2, MSTN, MTERFD1, MTMR14, MTMR3, MTOR, MTSS1, MYH11, MYLK, MYOM1, NBR1, NDUFB9, NEFM, NHLRC1, NME2, NPC1, NR2C2, NRBF2, NTHL1, NUP93, OBSCN, OPTN, P2RX5, PACS2, PARK2, PARK7, PDK1, PDK4, PEX13, PEX3, PFKP, PGK2, PHF23, PHYHIP, PI4K2A, PIK3C3, PIK3CA, PIK3CB, PIK3R4, PINK1, PLEKHM1, PLOD2, PNPO, PPARGC1A, PPY, PRKAA1, PRKAA2, PRKAB1, PRKAB2, PRKAG1, PRKAG2, PRKAG3, PRKD2, PRKG1, PSEN1, PTPN22, RAB12, RAB1A, RAB1B, RAB23, RAB24, RAB33B, RAB39, RAB7A, RB1CC1, RBM18, REEP2, REP15, RFWD3, RGS19, RHEB, RIMS3, RNF185, RNF41, RPS27A, RPTOR, RRAGA, RRAGB, RRAGC, RRAGD, S100A8, S100A9, SCN1A, SERPINB10, SESN2, SFRP4, SH3GLB1, SIRT2, SLC1A3, SLC1A4, SLC22A3, SLC25A19, SLC35B3, SLC35C1, SLC37A4, SLC6A1, SLCO1A2, SMURF1, SNAP29, SNAPIN, SNF8, SNRPB, SNRPB2, SNRPD1, SNRPF, SNTG1, SNX14, SPATA18, SQSTM1, SRPX, STAM, STAM2, STAT2, STBD1, STK11, STK32A, STOM, STX12, STX17, SUPT3H, TBC1D17, TBC1D25, TBC1D5, TCIRG1, TEAD4, TECPR1, TECPR2, TFEB, TM9SF1, TMBIM6, TMEM203, TMEM208, TMEM39A, TMEM39B, TMEM59, TMEM74, TMEM93, TNIK, TOLLIP, TOMM20, TOMM22, TOMM40, TOMM5, TOMM6, TOMM7, TOMM70A, TP53INP1, TP53INP2, TRAPPC8, TREM1, TRIM17, TRIM5, TSG101, TXLNA, UBA52, UBB, UBC, UBQLN1, UBQLN2, UBQLN4, ULK1, ULK2, ULK3, USP10, USP13, USP30, UVRAG, VAMP7, VAMP8, VDAC1, VMP1, VPS11, VPS16, VPS18, VPS25, VPS28, VPS33A, VPS33B, VPS36, VPS37A, VPS37B, VPS37C, VPS37D, VPS39, VPS41, VPS4A, VPS4B, VTA1, VTI1A, VTI1B, WDFY3, WDR45, WDR45L, WIPI1, WIPI2, XBP1, YIPF1, ZCCHC17, ZFYVE1, ZKSCAN3, ZNF189, ZNF593, and ZNF681. In some embodiments, the GeneSetScore (Up autophagy) is determined using RNA-seq, for example, single-cell RNA-seq (scRNA-seq), for example, as exemplified in Example 7 with respect to FIG. 25E. In some embodiments, the GeneSetScore (Up autophagy) is calculated by taking the mean log normalized gene expression value of all of the genes in the gene set.

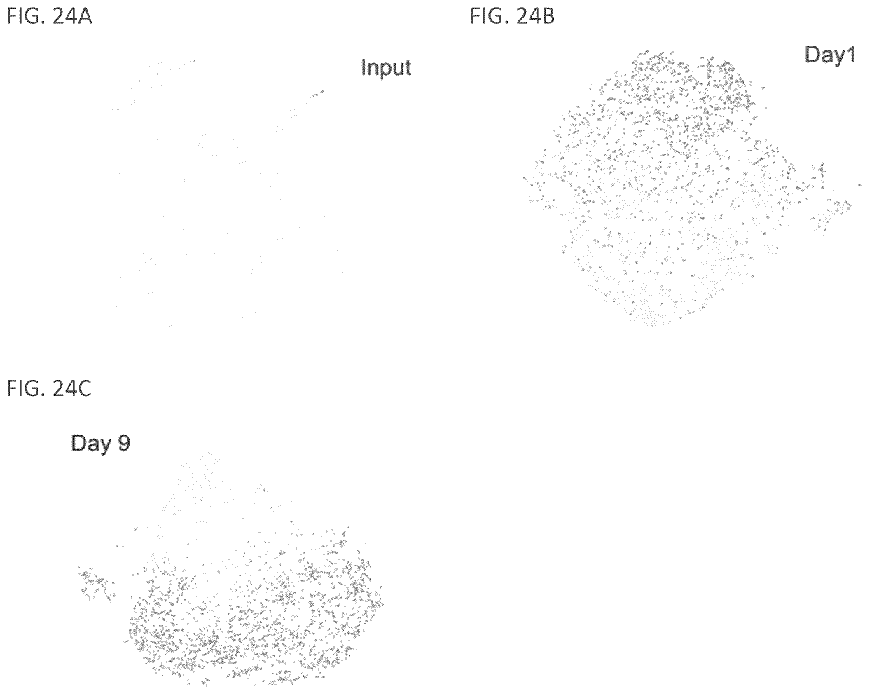

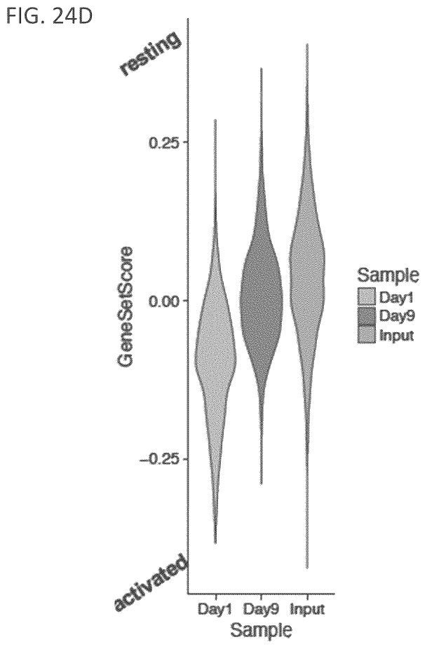

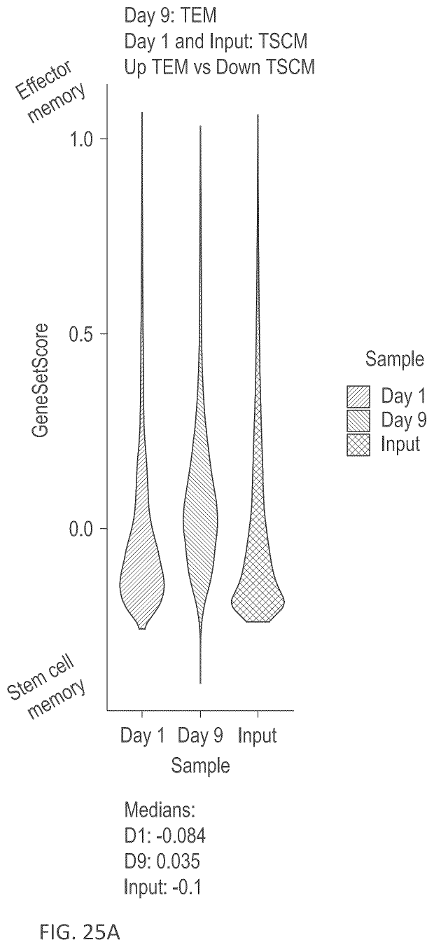

As used herein, the term “GeneSetScore (Up resting vs. Down activated)” of a cell refers to a score that reflects the degree at which the cell shows a resting T cell phenotype vs. an activated T cell phenotype. A higher GeneSetScore (Up resting vs. Down activated) indicates an increasing resting T cell phenotype, whereas a lower GeneSetScore (Up resting vs. Down activated) indicates an increasing activated T cell phenotype. In some embodiments, the GeneSetScore (Up resting vs. Down activated) is determined by measuring the expression of one or more genes that are up-regulated in resting T cells and/or down-regulated in activated T cells, for example, one or more genes selected from the group consisting of ABCA7, ABCF3, ACAP2, AMT, ANKH, ATF7IP2, ATG14, ATP1A1, ATXN7, ATXN7L3B, BCL7A, BEX4, BSDC1, BTG1, BTG2, BTN3A1, C11orf2l, C19orf22, C21orf2, CAMK2G, CARS2, CCNL2, CD248, CD5, CD55, CEP164, CHKB, CLK1, CLK4, CTSL1, DBP, DCUN1D2, DENND1C, DGKD, DLG1, DUSP1, EAPP, ECE1, ECHDC2, ERBB2IP, FAM117A, FAM134B, FAM134C, FAM169A, FAM190B, FAU, FLJ10038, FOXJ2, FOXJ3, FOXL1, FOXO1, FXYD5, FYB, HLA-E, HSPA1L, HYAL2, ICAM2, IFIT5, IFITM1, IKBKB, IQSEC1, IRS4, KIAA0664L3, KIAA0748, KLF3, KLF9, KRT18, LEFI, LINC00342, LIPA, LIPT1, LLGL2, LMBR1L, LPAR2, LTBP3, LYPD3, LZTFL1, MANBA, MAP2K6, MAP3K1, MARCH8, MAU2, MGEA5, MMP8, MPO, MSL1, MSL3, MYH3, MYLIP, NAGPA, NDST2, NISCH, NKTR, NLRP1, NOSIP, NPIP, NUMA1, PAIP2B, PAPD7, PBXIP1, PCIF1, PI4KA, PLCL2, PLEKHA1, PLEKHF2, PNISR, PPFIBP2, PRKCA, PRKCZ, PRKD3, PRMT2, PTP4A3, PXN, RASA2, RASA3, RASGRP2, RBM38, REPIN1, RNF38, RNF44, ROR1, RPL30, RPL32, RPLP1, RPS20, RPS24, RPS27, RPS6, RPS9, RXRA, RYK, SCAND2, SEMA4C, SETD1B, SETD6, SETX, SF3B1, SH2B1, SLC2A4RG, SLC35E2B, SLC46A3, SMAGP, SMARCE1, SMPD1, SNPH, SP140L, SPATA6, SPG7, SREK1IP1, SRSF5, STAT5B, SVIL, SYF2, SYNJ2BP, TAF1C, TBC1D4, TCF20, TECTA, TES, TMEM127, TMEM159, TMEM30B, TMEM66, TMEM8B, TP53TG1, TPCN1, TRIM22, TRIM44, TSC1, TSC22D1, TSC22D3, TSPYL2, TTC9, TTN, UBE2G2, USP33, USP34, VAMP1, VILL, VIPR1, VPS13C, ZBED5, ZBTB25, ZBTB40, ZC3H3, ZFP161, ZFP36L1, ZFP36L2, ZHX2, ZMYM5, ZNF136, ZNF148, ZNF318, ZNF350, ZNF512B, ZNF609, ZNF652, ZNF83, ZNF862, and ZNF91. In some embodiments, the GeneSetScore (Up resting vs. Down activated) is determined using RNA-seq, for example, single-cell RNA-seq (scRNA-seq), for example, as exemplified in Example 7 with respect to FIG. 24D. In some embodiments, the GeneSetScore (Up resting vs. Down activated) is calculated by taking the mean log normalized gene expression value of all of the genes in the gene set.

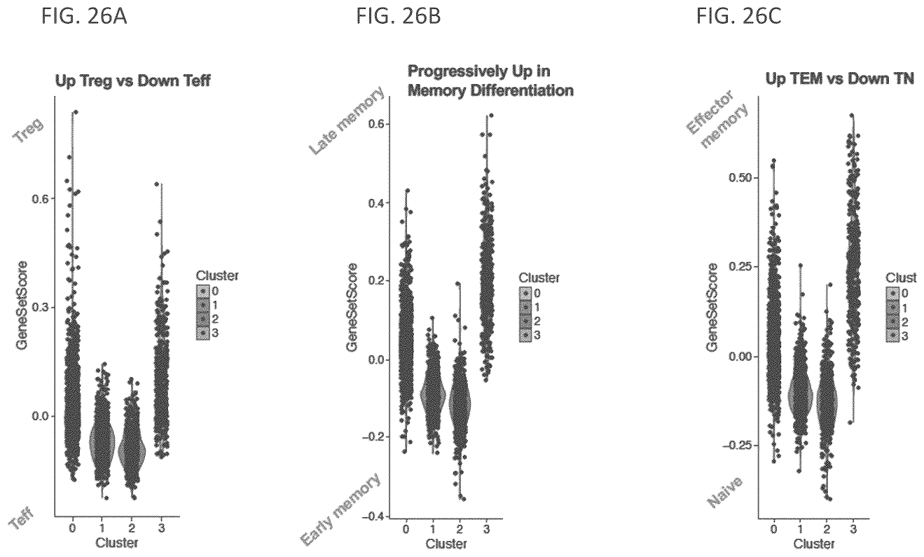

As used herein, the term “GeneSetScore (Progressively up in memory differentiation)” of a cell refers to a score that reflects the stage of the cell in memory differentiation. A higher GeneSetScore (Progressively up in memory differentiation) indicates an increasing late memory T cell phenotype, whereas a lower GeneSetScore (Progressively up in memory differentiation) indicates an increasing early memory T cell phenotype. In some embodiments, the GeneSetScore (Up autophagy) is determined by measuring the expression of one or more genes that are up-regulated during memory differentiation, for example, one or more genes selected from the group consisting of MTCH2, RAB6C, KIAA0195, SETD2, C2orf24, NRD1, GNA13, COPA, SELT, TNIP1, CBFA2T2, LRP10, PRKCI, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYOSA, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMIP, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYG1, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf2l, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, and KLRB1. In some embodiments, the GeneSetScore (Progressively up in memory differentiation) is determined using RNA-seq, for example, single-cell RNA-seq (scRNA-seq), for example, as exemplified in Example 7 with respect to FIG. 26B. In some embodiments, the GeneSetScore (Progressively up in memory differentiation) is calculated by taking the mean log normalized gene expression value of all of the genes in the gene set.

As used herein, the term “GeneSetScore (Up TEM vs. Down TN)” of a cell refers to a score that reflects the degree at which the cell shows an effector memory T cell (TEM) phenotype vs. a naïve T cell (TN) phenotype. A higher GeneSetScore (Up TEM vs. Down TN) indicates an increasing TEM phenotype, whereas a lower GeneSetScore (Up TEM vs. Down TN) indicates an increasing TN phenotype. In some embodiments, the GeneSetScore (Up TEM vs. Down TN) is determined by measuring the expression of one or more genes that are up-regulated in TEM cells and/or down-regulated in TN cells, for example, one or more genes selected from the group consisting of MYOSA, MXD4, STK3, S1PR5, GLCCI1, CCR3, SOX13, KRTAP5-2, PEA15, PARP8, RNF166, UEVLD, LIMK1, SLC6A6, SV2A, KPNA2, OSBPL7, ST7, GGA2, PI4K2A, CD68, ZAK, RORA, TGFBI, DNAJC1, JOSD1, ZFYVE28, LRP8, OSBPL3, CMIP, NAT13, TGFB1, ANTXR2, NR4A3, RDX, ADCY9, CHN1, CD300A, SCD5, PTPN22, LGALS1, RASGEF1A, GCNT1, GLUL, ABCA2, CLDND1, PAM, CLCF1, MXRA7, CLSTN3, ACOT9, METRNL, BMPR1A, LRIG1, APOBEC3G, CACNB3, RNF19A, RAB27A, FADS3, ACTN4, TBKBP1, FAM53B, MAN1A1, FAM38A, GRLF1, B4GALT5, WIPI1, DUSP2, ANXA5, AHNAK, CLIC1, MAP3K5, ST8SIA1, TARP, ADAM8, MATK, SLFN12L, PIK3R3, FAM46C, ANXA2P2, CTNNA1, NPC1, SH2D2A, ERN1, YPEL1, TBX21, STOM, PHACTR2, GBP5, ADRB2, PIK3AP1, DUSP10, PTGDR, EOMES, MAF, TPRG1, NBEAL2, NCAPH, SLC4A4, FOSL2, RGS2, TGFBR3, MYO1F, C17orf66, CYTH3, WEE1, PYHIN1, F2R, THBS1, CD58, AUTS2, FAM129A, TNIP3, GZMA, PRR5L, PRDM1, PLXND1, PTPRM, GFPT2, MYBL1, SLAMF7, ZEB2, CST7, CCL5, GZMK, and KLRB1. In some embodiments, the GeneSetScore (Up TEM vs. Down TN) is determined using RNA-seq, for example, single-cell RNA-seq (scRNA-seq), for example, as exemplified in Example 7 with respect to FIG. 26C. In some embodiments, the GeneSetScore (Up TEM vs. Down TN) is calculated by taking the mean log normalized gene expression value of all of the genes in the gene set.

In the context of GeneSetScore values (e.g., median GeneSetScore values), when a positive GeneSetScore is reduced by 100%, the value becomes 0. When a negative GeneSetScore is increased by 100%, the value becomes 0. For example, in FIG. 25A, the median GeneSetScore of the Day1 sample is −0.084; the median GeneSetScore of the Day9 sample is 0.035; and the median GeneSetScore of the input sample is −0.1. In FIG. 25A, increasing the median GeneSetScore of the input sample by 100% leads to a GeneSetScore value of 0; and increasing the median GeneSetScore of the input sample by 200% leads to a GeneSetScore value of 0.1. In FIG. 25A, decreasing the median GeneSetScore of the Day9 sample by 100% leads to a GeneSetScore value of 0; and decreasing the median GeneSetScore of the Day9 sample by 200% leads to a GeneSetScore value of −0.035.

As used herein, the term “bead” refers to a discrete particle with a solid surface, ranging in size from approximately 0.1 μm to several millimeters in diameter. Beads may be spherical (for example, microspheres) or have an irregular shape. Beads may comprise a variety of materials including, but not limited to, paramagnetic materials, ceramic, plastic, glass, polystyrene, methylstyrene, acrylic polymers, titanium, latex, Sepharose™, cellulose, nylon and the like. In some embodiments, the beads are relatively uniform, about 4.5 m in diameter, spherical, superparamagnetic polystyrene beads, for example, coated, for example, covalently coupled, with a mixture of antibodies against CD3 (for example, CD3 epsilon) and CD28. In some embodiments, the beads are Dynabeads®. In some embodiments, both anti-CD3 and anti-CD28 antibodies are coupled to the same bead, mimicking stimulation of T cells by antigen presenting cells. The property of Dynabeads® and the use of Dynabeads® for cell isolation and expansion are well known in the art, for example, see, Neurauter et al., Cell isolation and expansion using Dynabeads, Adv Biochem Eng Biotechnol. 2007; 106:41-73, herein incorporated by reference in its entirety.

As used herein, the term “nanomatrix” refers to a nanostructure comprising a matrix of mobile polymer chains. The nanomatrix is 1 to 500 nm, for example, 10 to 200 nm, in size. In some embodiments, the matrix of mobile polymer chains is attached to one or more agonists which provide activation signals to T cells, for example, agonist anti-CD3 and/or anti-CD28 antibodies. In some embodiments, the nanomatrix comprises a colloidal polymeric nanomatrix attached, for example, covalently attached, to an agonist of one or more stimulatory molecules and/or an agonist of one or more costimulatory molecules. In some embodiments, the agonist of one or more stimulatory molecules is a CD3 agonist (for example, an anti-CD3 agonistic antibody). In some embodiments, the agonist of one or more costimulatory molecules is a CD28 agonist (for example, an anti-CD28 agonistic antibody).

In some embodiments, the nanomatrix is characterized by the absence of a solid surface, for example, as the attachment point for the agonists, such as anti-CD3 and/or anti-CD28 antibodies. In some embodiments, the nanomatrix is the nanomatrix disclosed in WO2014/048920A1 or as given in the MACS® GMP T Cell TransAct™ kit from Miltenyi Biotcc GmbH, herein incorporated by reference in their entirety. MACS® GMP T Cell TransAct™ consists of a colloidal polymeric nanomatrix covalently attached to humanized recombinant agonist antibodies against human CD3 and CD28.

Various embodiments of the compositions and methods herein are described in further detail below. Additional definitions are set out throughout the specification.

### DESCRIPTION

Provided herein are compositions of matter and methods of use for the treatment of a disease such as cancer using cells expressing one or more chimeric antigen receptors (CARs). In some embodiments, the invention provides a cell (e.g., an immune effector cell, e.g., T cell or NK cell) engineered to express one or more CARs, wherein the CAR T cell (“CART”) or CAR NK cell exhibits an antitumor property.

In some embodiments, the cell expresses at least two CARs. In some embodiments, the cell expresses a first CAR that binds to a first antigen and a second CAR that binds to a second antigen. In some embodiments, the first antigen and the second antigen are different. In some embodiments, the first antigen is BCMA. In some embodiments, the first CAR is an anti-BCMA CAR comprising a CDR, VH, VL, scFv, or CAR sequence disclosed herein, e.g., a sequence disclosed in Tables 3-15, 19, 20, 22, and 26, or an amino acid sequence having at least about 85%, 90%, 95%, or 99% sequence identity thereto, e.g., an anti-BCMA CAR disclosed herein. In some embodiments, the second antigen is CD19. In some embodiments, the second antigen is an anti-CD19 CAR comprising a CDR, VH, VL, scFv, or CAR sequence disclosed herein, e.g., a sequence disclosed in Tables 2, 19, and 22, or an amino acid sequence having at least about 85%, 90%, 95%, or 99% sequence identity thereto, e.g., an anti-CD19 CAR disclosed herein. In some embodiments, the first CAR and the second CAR are expressed by nucleic acid sequences disposed on a single nucleic acid molecule. In some embodiments, the nucleic acid sequence encoding the first CAR and the nucleic acid sequence encoding the second CAR are separated by a nucleic acid sequence encoding a self-cleavage site, e.g., a P2A site, a T2A site, an E2A site, or an F2A site. In some embodiments, the cell is a cell expressing dual CARs disclosed herein. In some embodiments, the first CAR and the second CAR are expressed by nucleic acid sequences disposed on separate nucleic acid molecules. In some embodiments, the cell is engineered using a co-transduction system disclosed herein.

In some embodiments, the cell expresses a CAR that binds to a first antigen and a second antigen. In some embodiments, the CAR is a diabody CAR disclosed herein. In some embodiments, the CAR comprises a binding domain that comprises a first VH (VH1), a first VL (VL1), a second VH (VH2), and a second VL (VL2). In some embodiments, the VH1 ad VL1 bind to a first antigen and the VH2 and VL2 bind to a second antigen. In some embodiments, the VH1, VL1, VH2, and VL2 are arranged in the following configuration from the N-terminus to the C-terminus: VH1—optionally linker 1 (“L1”)-VH2—optionally linker 2 (“L2”)-VL2—optionally linker 3 (“L3”)-VL1, VH1—optionally L1-VL2—optionally L2-VH2—optionally L3-VL1, VL1—optionally L1-VH2—optionally L2-VL2—optionally L3-VH1, VL1—optionally L1-VL2—optionally L2-VH2—optionally L3-VH1, VH2—optionally L1-VH1—optionally L2-VL1—optionally L3-VL2, VH2—optionally L1-VL1—optionally L2-VH1—optionally L3-VL2, VL2—optionally L1-VH1—optionally L2-VL1—optionally L3-VH2; or VL2—optionally L1-VL1—optionally L2-VH1—optionally L3-VH2.

In some embodiments, the CARs of the invention combine an antigen binding domain with an intracellular signaling molecule. For example, in some embodiments, the intracellular signaling molecule includes, but is not limited to, CD3-zeta chain, 4-1BB and CD28 signaling modules and combinations thereof.

Furthermore, the present invention provides CAR compositions and their use in medicaments or methods for treating, among other diseases, cancer or any malignancy or autoimmune diseases.

### Chimeric Antigen Receptor (CAR)

The present invention provides immune effector cells (for example, T cells or NK cells) that are engineered to contain one or more CARs that direct the immune effector cells to cancer. This is achieved through an antigen binding domain on the CAR that is specific for a cancer associated antigen. There are two classes of cancer associated antigens (tumor antigens) that can be targeted by the CARs described herein: (1) cancer associated antigens that are expressed on the surface of cancer cells; and (2) cancer associated antigens that themselves are intracellular, however, fragments (peptides) of such antigens are presented on the surface of the cancer cells by MHC (major histocompatibility complex).

Accordingly, an immune effector cell, for example, obtained by a method described herein, can be engineered to contain a CAR that targets one of the following cancer associated antigens (tumor antigens): CD19, CD123, CD22, CD30, CD171, CS-1, CLL-1, CD33, EGFRvIII, GD2, GD3, BCMA, Tn Ag, PSMA, ROR1, FLT3, FAP, TAG72, CD38, CD44v6, CEA, EPCAM, B7H3, KIT, IL-13Ra2, Mesothelin, IL-11Ra, PSCA, VEGFR2, LewisY, CD24, PDGFR-beta, PRSS21, SSEA-4, CD20, Folate receptor alpha, ERBB2 (Her2/neu), MUC1, EGFR, NCAM, Prostase, PAP, ELF2M, Ephrin B2, IGF-1 receptor, CAIX, LMP2, gp100, bcr-abl, tyrosinase, EphA2, Fucosyl GM1, sLe, GM3, TGS5, HMWMAA, o-acetyl-GD2, Folate receptor beta, TEM1/CD248, TEM7R, CLDN6, TSHR, GPRC5D, CXORF61, CD97, CD179a, ALK, Plysialic acid, PLAC1, GloboH, NY-BR-1, UPK2, HAVCR1, ADRB3, PANX3, GPR20, LY6K, OR51E2, TARP, WT1, NY-ESO-1, LAGE-la, legumain, HPV E6, E7, MAGE-A1, MAGE Al, ETV6-AML, sperm protein 17, XAGE1, Tie 2, MAD-CT-1, MAD-CT-2, Fos-related antigen 1, p53, p53 mutant, prostein, survivin and telomerase, PCTA-1/Galectin 8, MelanA/MART1, Ras mutant, hTERT, sarcoma translocation breakpoints, ML-IAP, ERG (TMPRSS2 ETS fusion gene), NA17, PAX3, Androgen receptor, Cyclin B1, MYCN, RhoC, TRP-2, CYP1B1, BORIS, SART3, PAX5, OY-TES1, LCK, AKAP-4, SSX2, RAGE-1, human telomerase reverse transcriptase, RU1, RU2, intestinal carboxyl esterase, and mut hsp70-2.

Sequences of non-limiting examples of various components that can be part of a CAR molecule described herein are listed in Table 1, where “aa” stands for amino acids, and “na” stands for nucleic acids that encode the corresponding peptide.

TABLE 1
Sequences of various components of CAR

| SEQ ID NO | Description | Sequence |
|---|---|---|
| SEQ ID NO: | EF-1α promoter | CGTGAGGCTCCGGTGCCCGTCAGTGGGCAGAGCGCACATCGCC |
| 11 | (na) | CACAGTCCCCGAGAAGTTGGGGGGAGGGGTCGGCAATTGAAC |
| | | CGGTGCCTAGAGAAGGTGGCGCGGGGTAAACTGGGAAAGTGA |
| | | TGTCGTGTACTGGCTCCGCCTTTTTCCCGAGGGTGGGGGAGAA |
| | | CCGTATATAAGTGCAGTAGTCGCCGTGAACGTTCTTTTTCGCA |
| | | ACGGGTTTGCCGCCAGAACACAGGTAAGTGCCGTGTGTGGTTC |
| | | CCGCGGGCCTGGCCTCTTTACGGGTTATGGCCCTTGCGTGCCTT |
| | | GAATTACTTCCACCTGGCTGCAGTACGTGATTCTTGATCCCGA |
| | | GCTTCGGGTTGGAAGTGGGTGGGAGAGTTCGAGGCCTTGCGCT |
| | | TAAGGAGCCCCTTCGCCTCGTGCTTGAGTTGAGGCCTGGCCTG |
| | | GGCGCTGGGGCCGCCGCGTGCGAATCTGGTGGCACCTTCGCGC |
| | | CTGTCTCGCTGCTTTCGATAAGTCTCTAGCCATTTAAAATTTTT |
| | | GATGACCTGCTGCGACGCTTTTTTTCTGGCAAGATAGTCTTGTA |
| | | AATGCGGGCCAAGATCTGCACACTGGTATTTCGGTTTTTGGGG |
| | | CCGCGGGCGGCGACGGGGCCCGTGCGTCCCAGCGCACATGTTC |
| | | GGCGAGGCGGGGCCTGCGAGCGCGGCCACCGAGAATCGGACG |
| | | GGGGTAGTCTCAAGCTGGCCGGCCTGCTCTGGTGCCTGGCCTC |
| | | GCGCCGCCGTGTATCGCCCCGCCCTGGGCGGCAAGGCTGGCCC |
| | | GGTCGGCACCAGTTGCGTGAGCGGAAAGATGGCCGCTTCCCG |
| | | GCCCTGCTGCAGGGAGCTCAAAATGGAGGACGCGGCGCTCGG |
| | | GAGAGCGGGCGGGTGAGTCACCCACACAAAGGAAAAGGGCCT |
| | | TTCCGTCCTCAGCCGTCGCTTCATGTGACTCCACGGAGTACCG |
| | | GGCGCCGTCCAGGCACCTCGATTAGTTCTCGAGCTTTTGGAGT |
| | | ACGTCGTCTTTAGGTTGGGGGGAGGGGTTTTATGCGATGGAGT |
| | | TTCCCCACACTGAGTGGGTGGAGACTGAAGTTAGGCCAGCTTG |
| | | GCACTTGATGTAATTCTCCTTGGAATTTGCCCTTTTTGAGTTTG |
| | | GATCTTGGTTCATTCTCAAGCCTCAGACAGTGGTTCAAAGTTTT |
| | | TTTCTTCCATTTCAGGTGTCGTGA |
| SEQ ID NO: | Leader (aa) | MALPVTALLLPLALLLHAARP |
| 1 | | |
| SEQ ID NO: | Leader (na) | ATGGCCCTGCCTGTGACAGCCCTGCTGCTGCCTCTGGCTCTGCT |
| 12 | | GCTGCATGCCGCTAGACCC |
| SEQ ID NO: | Leader (na) | ATGGCCCTCCCTGTCACCGCCCTGCTGCTTCCGCTGGCTCTTCT |
| 199 | | GCTCCACGCCGCTCGGCCC |
| SEQ ID NO: | Leader (aa) | MLLLVTSLLLCELPHPAFLLIP |
| 351 | | |
| SEQ ID NO: | Leader (na) | ATGCTTCTCCTGGTGACAAGCCTTCTGCTCTGTGAGTTACCACA |
| 352 | | CCCAGCATTCCTCCTGATCCCA |
| SEQ ID NO: | Leader (na) | ATGCTGCTGCTGGTGACCAGCCTGCTGCTGTGCGAGCTGCCCC |
| 353 | | ACCCCGCCTTTCTGCTGATCCCC |
| SEQ ID NO: | CD 8 hinge (aa) | TTTPAPRPPTPAPTIASQPLSLRPEACRPAAGGAVHTRGLDFACD |
| 2 | | |
| SEQ ID NO: | CD8 hinge (na) | ACCACGACGCCAGCGCCGCGACCACCAACACCGGCGCCCACC |
| 13 | | ATCGCGTCGCAGCCCCTGTCCCTGCGCCCAGAGGCGTGCCGGC |
| | | CAGCGGCGGGGGGCGCAGTGCACACGAGGGGGCTGGACTTCG |
| | | CCTGTGAT |
| SEQ ID NO: | Ig4 hinge (aa) | ESKYGPPCPPCPAPEFLGGPSVFLFPPKPKDTLMISRTPEVTCVVV |
| 3 | | DVSQEDPEVQFNWYVDGVEVHNAKTKPREEQFNSTYRVVSVLT |
| | | VLHQDWLNGKEYKCKVSNKGLPSSIEKTISKAKGQPREPQVYTLP |
| | | PSQEEMTKNQVSLTCLVKGFYPSDIAVEWESNGQPENNYKTTPPV |
| | | LDSDGSFFLYSRLTVDKSRWQEGNVFSCSVMHEALHNHYTQKSL |
| | | SLSLGKM |
| SEQ ID NO: | Ig4 hinge (na) | GAGAGCAAGTACGGCCCTCCCTGCCCCCCTTGCCCTGCCCCCG |
| 14 | | AGTTCCTGGGCGGACCCAGCGTGTTCCTGTTCCCCCCCAAGCC |
| | | CAAGGACACCCTGATGATCAGCCGGACCCCCGAGGTGACCTGT |
| | | GTGGTGGTGGACGTGTCCCAGGAGGACCCCGAGGTCCAGTTCA |
| | | ACTGGTACGTGGACGGCGTGGAGGTGCACAACGCCAAGACCA |
| | | AGCCCCGGGAGGAGCAGTTCAATAGCACCTACCGGGTGGTGT |
| | | CCGTGCTGACCGTGCTGCACCAGGACTGGCTGAACGGCAAGG |
| | | AATACAAGTGTAAGGTGTCCAACAAGGGCCTGCCCAGCAGCA |
| | | TCGAGAAAACCATCAGCAAGGCCAAGGGCCAGCCTCGGGAGC |
| | | CCCAGGTGTACACCCTGCCCCCTAGCCAAGAGGAGATGACCA |
| | | AGAACCAGGTGTCCCTGACCTGCCTGGTGAAGGGCTTCTACCC |
| | | CAGCGACATCGCCGTGGAGTGGGAGAGCAACGGCCAGCCCGA |
| | | GAACAACTACAAGACCACCCCCCCTGTGCTGGACAGCGACGG |
| | | CAGCTTCTTCCTGTACAGCCGGCTGACCGTGGACAAGAGCCGG |
| | | TGGCAGGAGGGCAACGTCTTTAGCTGCTCCGTGATGCACGAGG |
| | | CCCTGCACAACCACTACACCCAGAAGAGCCTGAGCCTGTCCCT |
| | | GGGCAAGATG |
| SEQ ID NO: | IgD hinge (aa) | RWPESPKAQASSVPTAQPQAEGSLAKATTAPATTRNTGRGGEEK |
| 4 | | KKEKEKEEQEERETKTPECPSHTQPLGVYLLTPAVQDLWLRDKA |
| | | TFTCFVVGSDLKDAHLTWEVAGKVPTGGVEEGLLERHSNGSQSQ |
| | | HSRLTLPRSLWNAGTSVTCTLNHPSLPPQRLMALREPAAQAPVKL |
| | | SLNLLASSDPPEAASWLLCEVSGFSPPNILLMWLEDQREVNTSGF |
| | | APARPPPQPGSTTFWAWSVLRVPAPPSPQPATYTCVVSHEDSRTL |
| | | LNASRSLEVSYVTDH |
| SEQ ID NO: | IgD hinge (na) | AGGTGGCCCGAAAGTCCCAAGGCCCAGGCATCTAGTGTTCCTA |
| 15 | | CTGCACAGCCCCAGGCAGAAGGCAGCCTAGCCAAAGCTACTA |
| | | CTGCACCTGCCACTACGCGCAATACTGGCCGTGGCGGGGAGG |
| | | AGAAGAAAAAGGAGAAAGAGAAAGAAGAACAGGAAGAGAGG |
| | | GAGACCAAGACCCCTGAATGTCCATCCCATACCCAGCCGCTGG |
| | | GCGTCTATCTCTTGACTCCCGCAGTACAGGACTTGTGGCTTAG |
| | | AGATAAGGCCACCTTTACATGTTTCGTCGTGGGCTCTGACCTG |
| | | AAGGATGCCCATTTGACTTGGGAGGTTGCCGGAAAGGTACCCA |
| | | CAGGGGGGGTTGAGGAAGGGTTGCTGGAGCGCCATTCCAATG |
| | | GCTCTCAGAGCCAGCACTCAAGACTCACCCTTCCGAGATCCCT |
| | | GTGGAACGCCGGGACCTCTGTCACATGTACTCTAAATCATCCT |
| | | AGCCTGCCCCCACAGCGTCTGATGGCCCTTAGAGAGCCAGCCG |
| | | CCCAGGCACCAGTTAAGCTTAGCCTGAATCTGCTCGCCAGTAG |
| | | TGATCCCCCAGAGGCCGCCAGCTGGCTCTTATGCGAAGTGTCC |
| | | GGCTTTAGCCCGCCCAACATCTTGCTCATGTGGCTGGAGGACC |
| | | AGCGAGAAGTGAACACCAGCGGCTTCGCTCCAGCCCGGCCCC |
| | | CACCCCAGCCGGGTTCTACCACATTCTGGGCCTGGAGTGTCTT |
| | | AAGGGTCCCAGCACCACCTAGCCCCCAGCCAGCCACATACACC |
| | | TGTGTTGTGTCCCATGAAGATAGCAGGACCCTGCTAAATGCTT |
| | | CTAGGAGTCTGGAGGTTTCCTACGTGACTGACCATT |
| SEQ ID NO: | CD8 | IYIWAPLAGTCGVLLLSLVITLYC |
| 6 | Transmembrane | |
| | (aa) | |
| SEQ ID NO: | CD8 | ATCTACATCTGGGCGCCCTTGGCCGGGACTTGTGGGGTCCTTC |
| 17 | Transmembrane | TCCTGTCACTGGTTATCACCCTTTACTGC |
| | (na) | |
| SEQ ID NO: | 4-1BB intracellular | KRGRKKLLYIFKQPFMRPVQTTQEEDGCSCRFPEEEEGGCEL |
| 7 | domain (aa) | |
| SEQ ID NO: | 4-1BB intracellular | AAACGGGGCAGAAAGAAACTCCTGTATATATTCAAACAACCA |
| 18 | domain (na) | TTTATGAGACCAGTACAAACTACTCAAGAGGAAGATGGCTGTA |
| | | GCTGCCGATTTCCAGAAGAAGAAGAAGGAGGATGTGAACTG |
| SEQ ID NO: | CD27 (aa) | QRRKYRSNKGESPVEPAEPCRYSCPREEEGSTIPIQEDYRKPEPAC |
| 8 | | SP |
| SEQ ID NO: | CD27 (na) | AGGAGTAAGAGGAGCAGGCTCCTGCACAGTGACTACATGAAC |
| 19 | | ATGACTCCCCGCCGCCCCGGGCCCACCCGCAAGCATTACCAGC |
| | | CCTATGCCCCACCACGCGACTTCGCAGCCTATCGCTCC |
| SEQ ID NO: | CD3-zeta (aa) | RVKFSRSADAPAYKQGQNQLYNELNLGRREEYDVLDKRRGRDP |
| 9 | (Q/K mutant) | EMGGKPRRKNPQEGLYNELQKDKMAEAYSEIGMKGERRRGKGH |
| | | DGLYQGLSTATKDTYDALHMQALPPR |
| SEQ ID NO: | CD3-zeta (na) | AGAGTGAAGTTCAGCAGGAGCGCAGACGCCCCCGCGTACAAG |
| 20 | (Q/K mutant) | CAGGGCCAGAACCAGCTCTATAACGAGCTCAATCTAGGACGA |
| | | AGAGAGGAGTACGATGTTTTGGACAAGAGACGTGGCCGGGAC |
| | | CCTGAGATGGGGGGAAAGCCGAGAAGGAAGAACCCTCAGGAA |
| | | GGCCTGTACAATGAACTGCAGAAAGATAAGATGGCGGAGGCC |
| | | TACAGTGAGATTGGGATGAAAGGCGAGCGCCGGAGGGGCAAG |
| | | GGGCACGATGGCCTTTACCAGGGTCTCAGTACAGCCACCAAGG |
| | | ACACCTACGACGCCCTTCACATGCAGGCCCTGCCCCCTCGC |
| SEQ ID NO: | CD3-zeta (aa) | RVKFSRSADAPAYQQGQNQLYNELNLGRREEYDVLDKRRGRDP |
| 10 | (NCBI Reference | EMGGKPRRKNPQEGLYNELQKDKMAEAYSEIGMKGERRRGKGH |
| | Sequence | DGLYQGLSTATKDTYDALHMQALPPR |
| | NM_000734.3) | |
| SEQ ID NO: | CD3-zeta (na) | AGAGTGAAGTTCAGCAGGAGCGCAGACGCCCCCGCGTACCAG |
| 21 | (NCBI Reference | CAGGGCCAGAACCAGCTCTATAACGAGCTCAATCTAGGACGA |
| | Sequence | AGAGAGGAGTACGATGTTTTGGACAAGAGACGTGGCCGGGAC |
| | NM_000734.3) | CCTGAGATGGGGGGAAAGCCGAGAAGGAAGAACCCTCAGGAA |
| | | GGCCTGTACAATGAACTGCAGAAAGATAAGATGGCGGAGGCC |
| | | TACAGTGAGATTGGGATGAAAGGCGAGCGCCGGAGGGGCAAG |
| | | GGGCACGATGGCCTTTACCAGGGTCTCAGTACAGCCACCAAGG |
| | | ACACCTACGACGCCCTTCACATGCAGGCCCTGCCCCCTCGC |
| SEQ ID NO: | CD28 Intracellular | RSKRSRLLHSDYMNMTPRRPGPTRKHYQPYAPPRDFAAYRS |
| 36 | domain (amino acid | |
| | sequence) | |
| SEQ ID NO: | CD28 Intracellular | AGGAGTAAGAGGAGCAGGCTCCTGCACAGTGACTACATGAAC |
| 37 | domain (nucleotide | ATGACTCCCCGCCGCCCCGGGCCCACCCGCAAGCATTACCAGC |
| | sequence) | CCTATGCCCCACCACGCGACTTCGCAGCCTATCGCTCC |
| SEQ ID NO: | ICOS Intracellular | T K K K Y S S S V H D P N G E Y M F M R A V N T A K K S R L T D |
| 38 | domain (amino acid | V T L |
| | sequence) | |
| SEQ ID NO: | ICOS Intracellular | ACAAAAAAGAAGTATTCATCCAGTGTGCACGACCCTAACGGT |
| 39 | domain (nucleotide | GAATACATGTTCATGAGAGCAGTGAACACAGCCAAAAAATCC |
| | sequence) | AGACTCACAGATGTGACCCTA |
| SEQ ID NO: | GS hinge/linker | GGGGSGGGGS |
| 5 | (aa) | |
| SEQ ID NO: | GS hinge/linker | GGTGGCGGAGGTTCTGGAGGTGGAGGTTCC |
| 16 | (na) | |
| SEQ ID NO: | GS hinge/linker | GGTGGCGGAGGTTCTGGAGGTGGGGGTTCC |
| 40 | (na) | |
| SEQ ID NO: | linker | GGGGS |
| 25 | | |
| SEQ ID NO: | linker | (Gly-Gly-Gly-Gly-Ser)n, where n = 1-6, for example, GGGGSGGGGS |
| 26 | | GGGGSGGGGS GGGGSGGGGS |
| SEQ ID NO: | linker | GGGGSGGGGSGGGGSGGGGS |
| 27 | | |
| SEQ ID NO: | linker | GGGGSGGGGSGGGGS |
| 28 | | |
| SEQ ID NO: | linker | GGGS |
| 29 | | |
| SEQ ID NO: | linker | (Gly-Gly-Gly-Ser)n where n is a positive integer equal to or greater |
| 41 | | than 1 |
| SEQ ID NO: | linker | (Gly-Gly-Gly-Ser)n, where n = 1-10, for example, GGGSGGGSGG |
| 42 | | GSGGGSGGGS GGGSGGGSGG GSGGGSGGGS |
| SEQ ID NO: | linker | GSTSGSGKPGSGEGSTKG |
| 43 | | |
| SEQ ID NO: | poly(A) | (A)₅₀₀₀ |
| 30 | | This sequence may encompass 50-5000 adenines. |
| SEQ ID NO: | polyT | (T)₁₀₀ |
| 31 | | |
| SEQ ID NO: | polyT | (T)₅₀₀₀ |
| 32 | | This sequence may encompass 50-5000 thymines. |
| SEQ ID NO: | poly(A) | (A)₅₀₀₀ |
| 33 | | This sequence may encompass 100-5000 adenines. |
| SEQ ID NO: | poly(A) | (A)₄₀₀ |
| 34 | | This sequence may encompass 100-400 adenines. |
| SEQ ID NO: | poly(A) | (A)₂₀₀₀ |
| 35 | | This sequence may encompass 50-2000 adenines. |
| SEQ ID NO: | PD1 CAR (aa) | pgwfldspdrpwnpptfspallvvtegdnatftcsfsntsesfvlnwyrmspsnqtdklaafpedrsq |
| 22 | | pgqdcrfrvtqlpngrdfhmsvvrarrndsgtylcgaislapkaqikeslraelrvterraevptahpsp |
| | | sprpagqfqtlvtttpaprpptpaptiasqplslrpeacrpaaggavhtrgldfacdiyiwaplagtcgvl |
| | | llslvitlyckrgrkkllyifkqpfmrpvqttqeedgescrfpeeeeggcelrvkfsrsadapaykqgq |
| | | nqlynelnlgrreeydvldkrrgrdpemggkprrknpqeglynelqkdkmaeayseigmkgerrr |
| | | gkghdglyqglstatkdtydalhmqalppr |
| SEQ ID NO: | PD-1 CAR (na) | atggccctccctgtcactgccctgcttctccccctcgcactcctgctccacgccgctagaccacccggat |
| 23 | (PD1 ECD | ggtttctggactctccggatcgcccgtggaatcccccaaccttctcaccggcactcttggttgtgactgag |
| | underlined) | ggcgataatgcgaccttcacgtgctcgttctccaacacctccgaatcattcgtgctgaactggtaccgcat |
| | | gagcccgtcaaaccagaccgacaagctcgccgcgtttccggaagatcggtcgcaaccgggacaggat |
| | | tgtcggttccgcgtgactcaactgccgaatggcagagacttccacatgagcgtggtccgcgctaggcga |
| | | aacgactccgggacctacctgtgcggagccatctcgctggcgcctaaggcccaaatcaaagagagctt |
| | | gagggccgaactgagagtgaccgagcgcagagctgaggtgccaactgcacatccatccccatcgcct |
| | | cggcctgcggggcagtttcagaccctggtcacgaccactccggcgccgcgcccaccgactccggccc |
| | | caactatcgcgagccagcccctgtcgctgaggccggaagcatgccgccctgccgccggaggtgctgt |
| | | gcatacccggggattggacttcgcatgcgacatctacatttgggctcctctcgccggaacttgtggcgtg |
| | | ctccttctgtccctggtcatcaccctgtactgcaagcggggtcggaaaaagcttctgtacattttcaagca |
| | | gcccttcatgaggcccgtgcaaaccacccaggaggaggacggttgctcctgccggttccccgaagagg |
| | | aagaaggaggttgcgagctgcgcgtgaagttctcccggagcgccgacgcccccgcctataagcaggg |
| | | ccagaaccagctgtacaacgaactgaacctgggacggcgggaagagtacgatgtgctggacaagcg |
| | | gcgcggccgggaccccgaaatgggcgggaagcctagaagaaagaaccctcaggaaggcctgtataa |
| | | cgagctgcagaaggacaagatggccgaggcctactccgaaattgggatgaagggagagcggcgga |
| | | ggggaaaggggcacgacggcctgtaccaaggactgtccaccgccaccaaggacacatacgatgccc |
| | | tgcacatgcaggcccttccccctcgc |
| SEQ ID NO: | PD-1 CAR (aa) | Malpvtalllplalllhaarppgwfldspdrpwnpptfspallvvtegdnatftcsfsntsesfvlnwyr |
| 24 | with signal | mspsnqtdklaafpedrsqpgqdcrfrvtqlpngrdfhmsvvrarrndsgtylcgaislapkaqikes |
| | (PD1 ECD | lraelrvterraevptahpspsprpagqfqtlvtttpaprpptpaptiasqplslrpeacrpaaggavhtr |
| | underlined) | gldfacdiyiwaplagtcgvlllslvitlyckrgrkkllyifkqpfmrpvqttqeedgcscrfpeeeegg |
| | | celrvkfsrsadapaykqgqnqlynelnlgrreeydvldkrrgrdpemggkprrknpqeglynelq |
| | | kdkmaeayseigmkgerrrgkghdglyqglstatkdtydalhmqalppr |

In some embodiments the antigen binding domain comprises the extracellular domain, or a counter-ligand binding fragment thereof, of molecule that binds a counter ligand on the surface of a target cell.

The immune effector cells can comprise a recombinant DNA construct comprising sequences encoding a CAR, wherein the CAR comprises an antigen binding domain (for example, antibody or antibody fragment, TCR or TCR fragment) that binds specifically to a tumor antigen, for example, a tumor antigen described herein, and an intracellular signaling domain. The intracellular signaling domain can comprise a costimulatory signaling domain and/or a primary signaling domain, for example, a zeta chain. As described elsewhere, the methods described herein can include transducing a cell, for example, from the population of T regulatory-depleted cells, with a nucleic acid encoding a CAR, for example, a CAR described herein.

In some embodiments, a CAR comprises a scFv domain, wherein the scFv may be preceded by an optional leader sequence such as provided in SEQ ID NO: 1, and followed by an optional hinge sequence such as provided in SEQ ID NO:2 or SEQ ID NO:36 or SEQ ID NO:38, a transmembrane region such as provided in SEQ ID NO:6, an intracellular signaling domain that includes SEQ ID NO:7 or SEQ ID NO:16 and a CD3 zeta sequence that includes SEQ ID NO:9 or SEQ ID NO:10, for example, wherein the domains are contiguous with and in the same reading frame to form a single fusion protein.

In some embodiments, an exemplary CAR constructs comprise an optional leader sequence (for example, a leader sequence described herein), an extracellular antigen binding domain (for example, an antigen binding domain described herein), a hinge (for example, a hinge region described herein), a transmembrane domain (for example, a transmembrane domain described herein), and an intracellular stimulatory domain (for example, an intracellular stimulatory domain described herein). In some embodiments, an exemplary CAR construct comprises an optional leader sequence (for example, a leader sequence described herein), an extracellular antigen binding domain (for example, an antigen binding domain described herein), a hinge (for example, a hinge region described herein), a transmembrane domain (for example, a transmembrane domain described herein), an intracellular costimulatory signaling domain (for example, a costimulatory signaling domain described herein) and/or an intracellular primary signaling domain (for example, a primary signaling domain described herein).

An exemplary leader sequence is provided as SEQ ID NO: 1. Further exemplary leaders include those provided in SEQ ID NO: 351 or encoded by SEQ ID NOs: 352 or 353. An exemplary hinge/spacer sequence is provided as SEQ ID NO: 2 or SEQ ID NO:36 or SEQ ID NO:38. An exemplary transmembrane domain sequence is provided as SEQ ID NO:6. An exemplary sequence of the intracellular signaling domain of the 4-1BB protein is provided as SEQ ID NO: 7. An exemplary sequence of the intracellular signaling domain of CD27 is provided as SEQ ID NO:16. An exemplary CD3zeta domain sequence is provided as SEQ ID NO: 9 or SEQ ID NO:10.

In some embodiments, the immune effector cell comprises a recombinant nucleic acid construct comprising a nucleic acid molecule encoding a CAR, wherein the nucleic acid molecule comprises a nucleic acid sequence encoding an antigen binding domain, wherein the sequence is contiguous with and in the same reading frame as the nucleic acid sequence encoding an intracellular signaling domain. An exemplary intracellular signaling domain that can be used in the CAR includes, but is not limited to, one or more intracellular signaling domains of, for example, CD3-zeta, CD28, CD27, 4-1BB, and the like. In some instances, the CAR can comprise any combination of CD3-zeta, CD28, 4-1BB, and the like.

The nucleic acid sequences coding for the desired molecules can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

Nucleic acids encoding a CAR can be introduced into the immune effector cells using, for example, a retroviral or lentiviral vector construct.

Nucleic acids encoding a CAR can also be introduced into the immune effector cell using, for example, an RNA construct that can be directly transfected into a cell. A method for generating mRNA for use in transfection involves in vitro transcription (IVT) of a template with specially designed primers, followed by poly(A) addition, to produce a construct containing 3′ and 5′ untranslated sequence (“UTR”) (for example, a 3′ and/or 5′ UTR described herein), a 5′ cap (for example, a 5′ cap described herein) and/or Internal Ribosome Entry Site (IRES) (for example, an IRES described herein), the nucleic acid to be expressed, and a poly(A) tail, typically 50-2000 bases in length (for example, described in the Examples, for example, SEQ ID NO:35). RNA so produced can efficiently transfect different kinds of cells. In some embodiments, the template includes sequences for the CAR. In some embodiments, an RNA CAR vector is transduced into a cell, for example, a T cell by electroporation.

### Antigen Binding Domain

In some embodiments, a plurality of the immune effector cells, for example, the population of T regulatory-depleted cells, include a nucleic acid encoding a CAR that comprises a target-specific binding element otherwise referred to as an antigen binding domain. The choice of binding element depends upon the type and number of ligands that define the surface of a target cell. For example, the antigen binding domain may be chosen to recognize a ligand that acts as a cell surface marker on target cells associated with a particular disease state. Thus, examples of cell surface markers that may act as ligands for the antigen binding domain in a CAR described herein include those associated with viral, bacterial and parasitic infections, autoimmune disease and cancer cells.

In some embodiments, the portion of the CAR comprising the antigen binding domain comprises an antigen binding domain that targets a tumor antigen, for example, a tumor antigen described herein.

The antigen binding domain can be any domain that binds to the antigen including but not limited to a monoclonal antibody, a polyclonal antibody, a recombinant antibody, a human antibody, a humanized antibody, and a functional fragment thereof, including but not limited to a single-domain antibody such as a heavy chain variable domain (VH), a light chain variable domain (VL) and a variable domain (VHH) of camelid derived nanobody, and to an alternative scaffold known in the art to function as antigen binding domain, such as a recombinant fibronectin domain, a T cell receptor (TCR), or a fragment there of, for example, single chain TCR, and the like. In some instances, it is beneficial for the antigen binding domain to be derived from the same species in which the CAR will ultimately be used in. For example, for use in humans, it may be beneficial for the antigen binding domain of the CAR to comprise human or humanized residues for the antigen binding domain of an antibody or antibody fragment.

### CD19 Car

In some embodiments, the CAR-expressing cell described herein is a CD19 CAR-expressing cell (for example, a cell expressing a CAR that binds to human CD19).

In some embodiments, the antigen binding domain of the CD19 CAR has the same or a similar binding specificity as the FMC63 scFv fragment described in Nicholson et al. Mol. Immun. 34 (16-17): 1157-1165 (1997). In some embodiments, the antigen binding domain of the CD19 CAR includes the scFv fragment described in Nicholson et al. Mol. Immun. 34 (16-17): 1157-1165 (1997).

In some embodiments, the CD19 CAR includes an antigen binding domain (for example, a humanized antigen binding domain) according to Table 3 of WO2014/153270, incorporated herein by reference. WO2014/153270 also describes methods of assaying the binding and efficacy of various CAR constructs.

In some embodiments, the parental murine scFv sequence is the CAR19 construct provided in PCT publication WO2012/079000 (incorporated herein by reference). In some embodiments, the anti-CD19 binding domain is a scFv described in WO2012/079000.

In some embodiments, the CAR molecule comprises the fusion polypeptide sequence provided as SEQ ID NO: 12 in PCT publication WO2012/079000, which provides an scFv fragment of murine origin that specifically binds to human CD19.

In some embodiments, the CD19 CAR comprises an amino acid sequence provided as SEQ ID NO: 12 in PCT publication WO2012/079000.

### In Some Embodiments, the Amino Acid Sequence is:

Diqmtqttsslsaslgdrvtiscrasqdiskylnwyqqkpdgtvklliyhtsrlhsgvpsrfsgsgsgtdysltisnleqediatyfcqqgn tlpytfgggtkleitggggsggggsggggsevklgesgpglvapsqslsvtctvsgvslpdygvswirqpprkglewlgviwgsettyynsalksr ltiikdnsksqvflkmnslqtddtaiyycakhyyyggsyamdywgqgtsvtvsstttpaprpptpaptiasqplslrpeacrpaaggavhtrgldfa cdiyiwaplagtcgvlllslvitlyckrgrkkllyifkqpfmrpvqttqeedgcscrfpeeeeggcelrvkfsrsadapaykqgqnqlynelnlgrre eydvldkrrgrdpemggkprrknpgeglynelqkdkmaeayseigmkgerrrgkghdglyqglstatkdtydalhmqalppr (SEQ ID NO: 292), or a sequence substantially homologous thereto.

In some embodiments, the CD19 CAR has the USAN designation TISAGENLECLEUCEL-T. In embodiments, CTL019 is made by a gene modification of T cells is mediated by stable insertion via transduction with a self-inactivating, replication deficient Lentiviral (LV) vector containing the CTL019 transgene under the control of the EF-1 alpha promoter. CTL019 can be a mixture of transgene positive and negative T cells that are delivered to the subject on the basis of percent transgene positive T cells.

In other embodiments, the CD19 CAR comprises an antigen binding domain (for example, a humanized antigen binding domain) according to Table 3 of WO2014/153270, incorporated herein by reference.

Humanization of murine CD19 antibody is desired for the clinical setting, where the mouse-specific residues may induce a human-anti-mouse antigen (HAMA) response in patients who receive CART19 treatment, i.e., treatment with T cells transduced with the CAR19 construct. The production, characterization, and efficacy of humanized CD19 CAR sequences is described in International Application WO2014/153270 which is herein incorporated by reference in its entirety, including Examples 1-5 (p. 115-159).

In some embodiments, the CAR molecule is a humanized CD19 CAR comprising the amino acid sequence of:

(SEQ ID NO: 293)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSS

In some embodiments, the CAR molecule is a humanized CD19 CAR comprising the amino acid sequence of:

(SEQ ID NO: 294)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYKQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

In some embodiments, the CAR molecule is a humanized CD19 CAR comprising the amino acid sequence of:

(SEQ ID NO: 349)
MALPVTALLLPLALLLHAARPEIVMTQSPATLSLSPGERATLSCRASQDI
SKYLNWYQQKPGQAPRLLIYHTSRLHSGIPARFSGSGSGTDYTLTISSLQ
PEDFAVYFCQQGNTLPYTFGQGTKLEIKGGGGSGGGGSGGGGSQVQLQES
GPGLVKPSETLSLTCTVSGVSLPDYGVSWIRQPPGKGLEWIGVIWGSETT
YYQSSLKSRVTISKDNSKNQVSLKLSSVTAADTAVYYCAKHYYYGGSYAM
DYWGQGTLVTVSSTTTPAPRPPTPAPTIASQPLSLRPEACRPAAGGAVHT
RGLDFACDIYIWAPLAGTCGVLLLSLVITLYCKRGRKKLLYIFKQPFMRP
VQTTQEEDGCSCRFPEEEEGGCELRVKFSRSADAPAYQQGQNQLYNELNL
GRREEYDVLDKRRGRDPEMGGKPRRKNPQEGLYNELQKDKMAEAYSEIGM
KGERRRGKGHDGLYQGLSTATKDTYDALHMQALPPR

In some embodiments, the CAR molecule is a humanized CD19 CAR comprising the amino acid sequence of:

(SEQ ID NO: 350)
EIVMTQSPATLSLSPGERATLSCRASQDISKYLNWYQQKPGQAPRLLIYH
TSRLHSGIPARFSGSGSGTDYTLTISSLQPEDFAVYFCQQGNTLPYTFGQ
GTKLEIKGGGGSGGGGSGGGGSQVQLQESGPGLVKPSETLSLTCTVSGVS
LPDYGVSWIRQPPGKGLEWIGVIWGSETTYYQSSLKSRVTISKDNSKNQV
SLKLSSVTAADTAVYYCAKHYYYGGSYAMDYWGQGTLVTVSSTTTPAPRP
PTPAPTIASQPLSLRPEACRPAAGGAVHTRGLDFACDIYIWAPLAGTCGV
LLLSLVITLYCKRGRKKLLYIFKQPFMRPVQTTQEEDGCSCRFPEEEEGG
CELRVKFSRSADAPAYQQGQNQLYNELNLGRREEYDVLDKRRGRDPEMGG
KPRRKNPQEGLYNELQKDKMAEAYSEIGMKGERRRGKGHDGLYQGLSTAT
KDTYDALHMQALPPR

Any known CD19 CAR, for example, the CD19 antigen binding domain of any known CD19 CAR, in the art can be used in accordance with the present disclosure. For example, LG-740; CD19 CAR described in the U.S. Pat. Nos. 8,399,645; 7,446,190; Xu et al., Leuk Lymphoma. 2013 54(2):255-260(2012); Cruz et al., Blood 122(17):2965-2973 (2013); Brentjens et al., Blood, 118(18):4817-4828 (2011); Kochenderfer et al., Blood 116(20):4099-102 (2010); Kochenderfer et al., Blood 122 (25):4129-39(2013); and 16th Annu Meet Am Soc Gen Cell Ther (ASGCT) (May 15-18, Salt Lake City) 2013, Abst 10.

Exemplary CD19 CARs include CD19 CARs described herein or an anti-CD19 CAR described in Xu et al. Blood 123.24(2014):3750-9; Kochenderfer et al. Blood 122.25(2013):4129-39, Cruz et al. Blood 122.17(2013):2965-73, NCT00586391, NCT01087294, NCT02456350, NCT00840853, NCT02659943, NCT02650999, NCT02640209, NCT01747486, NCT02546739, NCT02656147, NCT02772198, NCT00709033, NCT02081937, NCT00924326, NCT02735083, NCT02794246, NCT02746952, NCT01593696, NCT02134262, NCT01853631, NCT02443831, NCT02277522, NCT02348216, NCT02614066, NCT02030834, NCT02624258, NCT02625480, NCT02030847, NCT02644655, NCT02349698, NCT02813837, NCT02050347, NCT01683279, NCT02529813, NCT02537977, NCT02799550, NCT02672501, NCT02819583, NCT02028455, NCT01840566, NCT01318317, NCT01864889, NCT02706405, NCT01475058, NCT01430390, NCT02146924, NCT02051257, NCT02431988, NCT01815749, NCT02153580, NCT01865617, NCT02208362, NCT02685670, NCT02535364, NCT02631044, NCT02728882, NCT02735291, NCT01860937, NCT02822326, NCT02737085, NCT02465983, NCT02132624, NCT02782351, NCT01493453, NCT02652910, NCT02247609, NCT01029366, NCT01626495, NCT02721407, NCT01044069, NCT00422383, NCT01680991, NCT02794961, or NCT02456207, each of which is incorporated herein by reference in its entirety.

In some embodiments, CD19 CARs comprise a sequence, for example, a CDR, VH, VL, scFv, or full-CAR sequence, disclosed in Table 2, or a sequence having at least 80%, 85%, 90%, 95%, or 99% identity thereto.

TABLE 2
Amino acid sequences of exemplary anti-CD19 molecules

| SEQ ID NO | Region | Sequence |
|---|---|---|
| CTL019 | | |
| 295 | HCDR1 | DYGVS |
| | (Kabat) | |
| 296 | HCDR2 | VIWGSETTYYNSALKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 301 | CTL019 | MALPVTALLLPLALLLHAARPDIQMTQTTSSLSASLGDRVTISCRASQDIS |
| | Full amino | KYLNWYQQKPDGTVKLLIYHTSRLHSGVPSRFSGSGSGTDYSLTISNLEQ |
| | acid | EDIATYFCQQGNTLPYTFGGGTKLEITGGGGSGGGGSGGGGSEVKLQES |
| | sequence | GPGLVAPSQSLSVTCTVSGVSLPDYGVSWIRQPPRKGLEWLGVIWGSET |
| | | TYYNSALKSRLTIIKDNSKSQVFLKMNSLQTDDTAIYYCAKHYYYGGSY |
| | | AMDYWGQGTSVTVSSTTTPAPRPPTPAPTIASQPLSLRPEACRPAAGGAV |
| | | HTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNE |
| | | LNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 302 | CTL019 | ATGGCCTTACCAGTGACCGCCTTGCTCCTGCCGCTGGCCTTGCTGCTC |
| | Full | CACGCCGCCAGGCCGGACATCCAGATGACACAGACTACATCCTCCCT |
| | nucleotide | GTCTGCCTCTCTGGGAGACAGAGTCACCATCAGTTGCAGGGCAAGTC |
| | sequence | AGGACATTAGTAAATATTTAAATTGGTATCAGCAGAAACCAGATGGA |
| | | ACTGTTAAACTCCTGATCTACCATACATCAAGATTACACTCAGGAGTC |
| | | CCATCAAGGTTCAGTGGCAGTGGGTCTGGAACAGATTATTCTCTCACC |
| | | ATTAGCAACCTGGAGCAAGAAGATATTGCCACTTACTTTTGCCAACA |
| | | GGGTAATACGCTTCCGTACACGTTCGGAGGGGGGACCAAGCTGGAGA |
| | | TCACAGGTGGCGGTGGCTCGGGCGGTGGTGGGTCGGGTGGCGGCGGA |
| | | TCTGAGGTGAAACTGCAGGAGTCAGGACCTGGCCTGGTGGCGCCCTC |
| | | ACAGAGCCTGTCCGTCACATGCACTGTCTCAGGGGTCTCATTACCCG |
| | | ACTATGGTGTAAGCTGGATTCGCCAGCCTCCACGAAAGGGTCTGGAG |
| | | TGGCTGGGAGTAATATGGGGTAGTGAAACCACATACTATAATTCAGC |
| | | TCTCAAATCCAGACTGACCATCATCAAGGACAACTCCAAGAGCCAAG |
| | | TTTTCTTAAAAATGAACAGTCTGCAAACTGATGACACAGCCATTTACT |
| | | ACTGTGCCAAACATTATTACTACGGTGGTAGCTATGCTATGGACTACT |
| | | GGGGCCAAGGAACCTCAGTCACCGTCTCCTCAACCACGACGCCAGCG |
| | | CCGCGACCACCAACACCGGCGCCCACCATCGCGTCGCAGCCCCTGTC |
| | | CCTGCGCCCAGAGGCGTGCCGGCCAGCGGCGGGGGGCGCAGTGCAC |
| | | ACGAGGGGGCTGGACTTCGCCTGTGATATCTACATCTGGGCGCCCTT |
| | | GGCCGGGACTTGTGGGGTCCTTCTCCTGTCACTGGTTATCACCCTTTA |
| | | CTGCAAACGGGGCAGAAAGAAACTCCTGTATATATTCAAACAACCAT |
| | | TTATGAGACCAGTACAAACTACTCAAGAGGAAGATGGCTGTAGCTGC |
| | | CGATTTCCAGAAGAAGAAGAAGGAGGATGTGAACTGAGAGTGAAGT |
| | | TCAGCAGGAGCGCAGACGCCCCCGCGTACAAGCAGGGCCAGAACCA |
| | | GCTCTATAACGAGCTCAATCTAGGACGAAGAGAGGAGTACGATGTTT |
| | | TGGACAAGAGACGTGGCCGGGACCCTGAGATGGGGGGAAAGCCGAG |
| | | AAGGAAGAACCCTCAGGAAGGCCTGTACAATGAACTGCAGAAAGAT |
| | | AAGATGGCGGAGGCCTACAGTGAGATTGGGATGAAAGGCGAGCGCC |
| | | GGAGGGGCAAGGGGCACGATGGCCTTTACCAGGGTCTCAGTACAGCC |
| | | ACCAAGGACACCTACGACGCCCTTCACATGCAGGCCCTGCCCCCTCGC |
| 303 | CTL019 | DIQMTQTTSSLSASLGDRVTISCRASQDISKYLNWYQQKPDGTVKLLIYH |
| | scFv | TSRLHSGVPSRFSGSGSGTDYSLTISNLEQEDIATYFCQQGNTLPYTFGGG |
| | domain | TKLEITGGGGSGGGGSGGGGSEVKLQESGPGLVAPSQSLSVTCTVSGVSL |
| | | PDYGVSWIRQPPRKGLEWLGVIWGSETTYYNSALKSRLTIIKDNSKSQVF |
| | | LKMNSLQTDDTAIYYCAKHYYYGGSYAMDYWGQGTSVTVSS |
| Humanized | | |
| CAR2 | | |
| 295 | HCDR1 | DYGVS |
| | (Kabat) | |
| 304 | HCDR2 | VIWGSETTYYQSSLKS |
| | (Kabat) | |
| 297 | HCDR3 | HYYYGGSYAMDY |
| | (Kabat) | |
| 298 | LCDR1 | RASQDISKYLN |
| | (Kabat) | |
| 299 | LCDR2 | HTSRLHS |
| | (Kabat) | |
| 300 | LCDR3 | QQGNTLPYT |
| | (Kabat) | |
| 293 | CAR2 scFv | EIVMTQSPATLSLSPGERATLSCRASQDISKYLNWYQQKPGQAPRLLIYH |
| | domain - aa | TSRLHSGIPARFSGSGSGTDYTLTISSLQPEDFAVYFCQQGNTLPYTFGQG |
| | (Linker is | TKLEIKGGGGSGGGGSGGGGSQVQLQESGPGLVKPSETLSLTCTVSGVS |
| | underlined) | LPDYGVSWIRQPPGKGLEWIGVIWGSETTYYQSSLKSRVTISKDNSKNQ |
| | | VSLKLSSVTAADTAVYYCAKHYYYGGSYAMDYWGQGTLVTVSS |
| 305 | CAR2 scFv | GAAATTGTGATGACCCAGTCACCCGCCACTCTTAGCCTTTCACCCGGT |
| | domain - nt | GAGCGCGCAACCCTGTCTTGCAGAGCCTCCCAAGACATCTCAAAATA |
| | | CCTTAATTGGTATCAACAGAAGCCCGGACAGGCTCCTCGCCTTCTGAT |
| | | CTACCACACCAGCCGGCTCCATTCTGGAATCCCTGCCAGGTTCAGCG |
| | | GTAGCGGATCTGGGACCGACTACACCCTCACTATCAGCTCACTGCAG |
| | | CCAGAGGACTTCGCTGTCTATTTCTGTCAGCAAGGGAACACCCTGCC |
| | | CTACACCTTTGGACAGGGCACCAAGCTCGAGATTAAAGGTGGAGGTG |
| | | GCAGCGGAGGAGGTGGGTCCGGCGGTGGAGGAAGCCAGGTCCAACT |
| | | CCAAGAAAGCGGACCGGGTCTTGTGAAGCCATCAGAAACTCTTTCAC |
| | | TGACTTGTACTGTGAGCGGAGTGTCTCTCCCCGATTACGGGGTGTCTT |
| | | GGATCAGACAGCCACCGGGGAAGGGTCTGGAATGGATTGGAGTGATT |
| | | TGGGGCTCTGAGACTACTTACTACCAATCATCCCTCAAGTCACGCGTC |
| | | ACCATCTCAAAGGACAACTCTAAGAATCAGGTGTCACTGAAACTGTC |
| | | ATCTGTGACCGCAGCCGACACCGCCGTGTACTATTGCGCTAAGCATT |
| | | ACTATTATGGCGGGAGCTACGCAATGGATTACTGGGGACAGGGTACT |
| | | CTGGTCACCGTGTCCAGC |
| 306 | CAR 2 - | MALPVTALLLPLALLLHAARPEIVMTQSPATLSLSPGERATLSCRASQDIS |
| | Full - aa | KYLNWYQQKPGQAPRLLIYHTSRLHSGIPARFSGSGSGTDYTLTISSLQPE |
| | | DFAVYFCQQGNTLPYTFGQGTKLEIKGGGGSGGGGSGGGGSQVQLQES |
| | | GPGLVKPSETLSLTCTVSGVSLPDYGVSWIRQPPGKGLEWIGVIWGSETT |
| | | YYQSSLKSRVTISKDNSKNQVSLKLSSVTAADTAVYYCAKHYYYGGSY |
| | | AMDYWGQGTLVTVSSTTTPAPRPPTPAPTIASQPLSLRPEACRPAAGGAV |
| | | HTRGLDFACDIYIWAPLAGTCGVLLLSLVITLYCKRGRKKLLYIFKQPFM |
| | | RPVQTTQEEDGCSCRFPEEEEGGCELRVKFSRSADAPAYKQGQNQLYNE |
| | | LNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 307 | CAR 2 - | atggccctccctgtcaccgccctgctgcttccgctggctcttctgctccacgccgctcggcccgaaattgtgatgacc |
| | Full - nt | cagtcacccgccactcttagcctttcacccggtgagcgcgcaaccctgtcttgcagagcctcccaagacatctcaaa |
| | | ataccttaattggtatcaacagaagcccggacaggctcctcgccttctgatctaccacaccagccggctccattctgg |
| | | aatccctgccaggttcagcggtagcggatctgggaccgactacaccctcactatcagctcactgcagccagagga |
| | | cttcgctgtctatttctgtcagcaagggaacaccctgccctacacctttggacagggcaccaagctcgagattaaag |
| | | gtggaggtggcagcggaggaggtgggtccggcggtggaggaagccaggtccaactccaagaaagcggaccg |
| | | ggtcttgtgaagccatcagaaactctttcactgacttgtactgtgagcggagtgtctctccccgattacggggtgtctt |
| | | ggatcagacagccaccggggaagggtctggaatggattggagtgatttggggctctgagactacttactaccaatc |
| | | atccctcaagtcacgcgtcaccatctcaaaggacaactctaagaatcaggtgtcactgaaactgtcatctgtgaccg |
| | | cagccgacaccgccgtgtactattgcgctaagcattactattatggcgggagctacgcaatggattactggggaca |
| | | gggtactctggtcaccgtgtccagcaccactaccccagcaccgaggccacccaccccggctcctaccatcgcctc |
| | | ccagcctctgtccctgcgtccggaggcatgtagacccgcagctggtggggccgtgcatacccggggtcttgacttc |
| | | gcctgcgatatctacatttgggcccctctggctggtacttgcggggtcctgctgctttcactcgtgatcactctttact |
| | | gtaagcgcggtcggaagaagctgctgtacatctttaagcaacccttcatgaggcctgtgcagactactcaagaggagg |
| | | acggctgttcatgccggttcccagaggaggaggaaggggctgcgaactgcgcgtgaaattcagccgcagcgca |
| | | gatgctccagcctacaagcaggggcagaaccagctctacaacgaactcaatcttggtcggagagaggagtacga |
| | | cgtgctggacaagcggagaggacgggacccagaaatggggggaagccgcgcagaaagaatccccaagagg |
| | | gcctgtacaacgagctccaaaaggataagatggcagaagcctatagcgagattggtatgaaaggggaacgcaga |
| | | agaggcaaaggccacgacggactgtaccagggactcagcaccgccaccaaggacacctatgacgctcttcacat |
| | | gcaggccctgccgcctcgg |
| 349 | CAR 2A- | MALPVTALLLPLALLLHAARPEIVMTQSPATLSLSPGERATLSCRASQDIS |
| | Full amino | KYLNWYQQKPGQAPRLLIYHTSRLHSGIPARFSGSGSGTDYTLTISSLQPE |
| | acid | DFAVYFCQQGNTLPYTFGQGTKLEIKGGGGSGGGGSGGGGSQVQLQES |
| | sequence; | GPGLVKPSETLSLTCTVSGVSLPDYGVSWIRQPPGKGLEWIGVIWGSETT |
| | signal | YYQSSLKSRVTISKDNSKNQVSLKLSSVTAADTAVYYCAKHYYYGGSY |
| | peptide | AMDYWGQGTLVTVSSTTTPAPRPPTPAPTIASQPLSLRPEACRPAAGGAV |
| | underlined | HTRGLDFACDIYIWAPLAGTCGVLLLSLVITLYCKRGRKKLLYIFKQPFM |
| | | RPVQTTQEEDGCSCRFPEEEEGGCELRVKFSRSADAPAYQQGQNQLYNE |
| | | LNLGRREEYDVLDKRRGRDPEMGGKPRRKNPQEGLYNELQKDKMAEA |
| | | YSEIGMKGERRRGKGHDGLYQGLSTATKDTYDALHMQALPPR |
| 225 | CAR 2A - | EIVMTQSPATLSLSPGERATLSCRASQDISKYLNWYQQKPGQAPRLLIYH |
| | amino acid | TSRLHSGIPARFSGSGSGTDYTLTISSLQPEDFAVYFCQQGNTLPYTFGQG |
| | sequence; | TKLEIKGGGGSGGGGSGGGGSQVQLQESGPGLVKPSETLSLTCTVSGVS |
| | no signal | LPDYGVSWIRQPPGKGLEWIGVIWGSETTYYQSSLKSRVTISKDNSKNQ |
| | peptide | VSLKLSSVTAADTAVYYCAKHYYYGGSYAMDYWGQGTLVTVSSTTTP |
| | | APRPPTPAPTIASQPLSLRPEACRPAAGGAVHTRGLDFACDIYIWAPLAG |
| | | TCGVLLLSLVITLYCKRGRKKLLYIFKQPFMRPVQTTQEEDGCSCRFPEE |
| | | EEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDG |
| | | LYQGLSTATKDTYDALHMQALPPR |
| 354 | CAR 2A | atggccctccctgtcaccgccctgctgcttccgctggctcttctgctccacgccgctcggcccgaaattgtgatgacc |
| | full nucleic | cagtcacccgccactcttagcctttcacccggtgagcgcgcaaccctgtcttgcagagcctcccaagacatctcaaa |
| | acid | ataccttaattggtatcaacagaagcccggacaggctcctcgccttctgatctaccacaccagccggctccattctgg |
| | sequence; | aatccctgccaggttcagcggtagcggatctgggaccgactacaccctcactatcagctcactgcagccagagga |
| | signal | cttcgctgtctatttctgtcagcaagggaacaccctgccctacacctttggacagggcaccaagctcgagattaaag |
| | peptide and | gtggaggtggcagcggaggaggtgggtccggcggtggaggaagccaggtccaactccaagaaagcggaccg |
| | stop codon | ggtcttgtgaagccatcagaaactctttcactgacttgtactgtgagcggagtgtctctccccgattacggggtgtctt |
| | underlined | ggatcagacagccaccggggaagggtctggaatggattggagtgatttggggctctgagactacttactaccaatc |
| | | atccctcaagtcacgcgtcaccatctcaaaggacaactctaagaatcaggtgtcactgaaactgtcatctgtgaccg |
| | | cagccgacaccgccgtgtactattgcgctaagcattactattatggcgggagctacgcaatggattactggggaca |
| | | gggtactctggtcaccgtgtccagcaccactaccccagcaccgaggccacccaccccggctcctaccatcgcctc |
| | | ccagcctctgtccctgcgtccggaggcatgtagacccgcagctggtggggccgtgcatacccggggtcttgacttc |
| | | gcctgcgatatctacatttgggcccctctggctggtacttgcggggtcctgctgctttcactcgtgatcactctttact |
| | | gtaagcgcggtcggaagaagctgctgtacatctttaagcaacccttcatgaggcctgtgcagactactcaagaggagg |
| | | acggctgttcatgccggttcccagaggaggaggaaggggctgcgaactgcgcgtgaaattcagccgcagcgca |
| | | gatgctccagcctaccagcaggggcagaaccagctctacaacgaactcaatcttggtcggagagaggagtacga |
| | | cgtgctggacaagcggagaggacgggacccagaaatggggggaagccgcgcagaaagaatccccaagagg |
| | | gcctgtacaacgagctccaaaaggataagatggcagaagcctatagcgagattggtatgaaaggggaacgcaga |
| | | agaggcaaaggccacgacggactgtaccagggactcagcaccgccaccaaggacacctatgacgctcttcacat |
| | | gcaggccctgccgcctcggtaa |
| 355 | CAR 2A | atggccctccctgtcaccgccctgctgcttccgctggctcttctgctccacgccgctcggcccgaaattgtgatgacc |
| | nucleic | cagtcacccgccactcttagcctttcacccggtgagcgcgcaaccctgtcttgcagagcctcccaagacatctcaaa |
| | acid | ataccttaattggtatcaacagaagcccggacaggctcctcgccttctgatctaccacaccagccggctccattctgg |
| | sequence; | aatccctgccaggttcagcggtagcggatctgggaccgactacaccctcactatcagctcactgcagccagagga |
| | signal | cttcgctgtctatttctgtcagcaagggaacaccctgccctacacctttggacagggcaccaagctcgagattaaag |
| | peptide | gtggaggtggcagcggaggaggtgggtccggcggtggaggaagccaggtccaactccaagaaagcggaccg |
| | underlined; | ggtcttgtgaagccatcagaaactctttcactgacttgtactgtgagcggagtgtctctccccgattacggggtgtctt |
| | no stop | ggatcagacagccaccggggaagggtctggaatggattggagtgatttggggctctgagactacttactaccaatc |
| | codon | atccctcaagtcacgcgtcaccatctcaaaggacaactctaagaatcaggtgtcactgaaactgtcatctgtgaccg |
| | | cagccgacaccgccgtgtactattgcgctaagcattactattatggcgggagctacgcaatggattactggggaca |
| | | gggtactctggtcaccgtgtccagcaccactaccccagcaccgaggccacccaccccggctcctaccatcgcctc |
| | | ccagcctctgtccctgcgtccggaggcatgtagacccgcagctggtggggccgtgcatacccggggtcttgacttc |
| | | gcctgcgatatctacatttgggcccctctggctggtacttgcggggtcctgctgctttcactcgtgatcactctttact |
| | | gtaagcgcggtcggaagaagctgctgtacatctttaagcaacccttcatgaggcctgtgcagactactcaagaggagg |
| | | acggctgttcatgccggttcccagaggaggaggaaggggctgcgaactgcgcgtgaaattcagccgcagcgca |
| | | gatgctccagcctaccagcaggggcagaaccagctctacaacgaactcaatcttggtcggagagaggagtacga |
| | | cgtgctggacaagcggagaggacgggacccagaaatgggcgggaagccgcgcagaaagaatccccaagagg |
| | | gcctgtacaacgagctccaaaaggataagatggcagaagcctatagcgagattggtatgaaaggggaacgcaga |
| | | agaggcaaaggccacgacggactgtaccagggactcagcaccgccaccaaggacacctatgacgctcttcacat |
| | | gcaggccctgccgcctcgg |
| 356 | CAR 2A | gaaattgtgatgacccagtcacccgccactcttagcctttcacccggtgagcgcgcaaccctgtcttgcagagcctc |
| | nucleic | ccaagacatctcaaaataccttaattggtatcaacagaagcccggacaggctcctcgccttctgatctaccacacca |
| | acid | gccggctccattctggaatccctgccaggttcagcggtagcggatctgggaccgactacaccctcactatcagctc |
| | sequence; | actgcagccagaggacttcgctgtctatttctgtcagcaagggaacaccctgccctacacctttggacagggcacca |
| | no signal | agctcgagattaaaggtggaggtggcagcggaggaggtgggtccggcggtggaggaagccaggtccaactcca |
| | peptide; | agaaagcggaccgggtcttgtgaagccatcagaaactctttcactgacttgtactgtgagcggagtgtctctccccg |
| | stop codon | attacggggtgtcttggatcagacagccaccggggaagggtctggaatggattggagtgatttggggctctgagac |
| | underlined | tacttactaccaatcatccctcaagtcacgcgtcaccatctcaaaggacaactctaagaatcaggtgtcactgaaact |
| | | gtcatctgtgaccgcagccgacaccgccgtgtactattgcgctaagcattactattatggcgggagctacgcaatgg |
| | | attactggggacagggtactctggtcaccgtgtccagcaccactaccccagcaccgaggccacccaccccggctc |
| | | ctaccatcgcctcccagcctctgtccctgcgtccggaggcatgtagacccgcagctggtggggccgtgcataccc |
| | | ggggtcttgacttcgcctgcgatatctacatttgggcccctctggctggtacttgcggggtcctgctgctttcactcgt |
| | | gatcactctttactgtaagcgcggtcggaagaagctgctgtacatctttaagcaacccttcatgaggcctgtgcagact |
| | | actcaagaggaggacggctgttcatgccggttcccagaggaggaggaaggcggctgcgaactgcgcgtgaaatt |
| | | cagccgcagcgcagatgctccagcctaccagcaggggcagaaccagctctacaacgaactcaatcttggtcgga |
| | | gagaggagtacgacgtgctggacaagcggagaggacgggacccagaaatgggcgggaagccgcgcagaaa |
| | | gaatccccaagagggcctgtacaacgagctccaaaaggataagatggcagaagcctatagcgagattggtatgaa |
| | | aggggaacgcagaagaggcaaaggccacgacggactgtaccagggactcagcaccgccaccaaggacaccta |
| | | tgacgctcttcacatgcaggccctgccgcctcggtaa |
| SEQ ID NO: | CAR 2A | gaaattgtgatgacccagtcacccgccactcttagcctttcacccggtgagcgcgcaaccctgtcttgca |
| 417 | nucleic | gagcctcccaagacatctcaaaataccttaattggtatcaacagaagcccggacaggctcctcgccttct |
| | acid | gatctaccacaccagccggctccattctggaatccctgccaggttcagcggtagcggatctgggaccga |
| | sequence; | ctacaccctcactatcagctcactgcagccagaggacttcgctgtctatttctgtcagcaagggaacaccc |
| | no signal | tgccctacacctttggacagggcaccaagctcgagattaaaggtggaggtggcagcggaggaggtgg |
| | peptide; no | gtccggcggtggaggaagccaggtccaactccaagaaagcggaccgggtcttgtgaagccatcagaa |
| | stop codon | actctttcactgacttgtactgtgagcggagtgtctctccccgattacggggtgtcttggatcagacagcca |
| | | ccggggaagggtctggaatggattggagtgatttggggctctgagactacttactaccaatcatccctca |
| | | agtcacgcgtcaccatctcaaaggacaactctaagaatcaggtgtcactgaaactgtcatctgtgaccgc |
| | | agccgacaccgccgtgtactattgcgctaagcattactattatggcgggagctacgcaatggattactgg |
| | | ggacagggtactctggtcaccgtgtccagcaccactaccccagcaccgaggccacccaccccggctc |
| | | ctaccatcgcctcccagcctctgtccctgcgtccggaggcatgtagacccgcagctggtggggccgtg |
| | | catacccggggtcttgacttcgcctgcgatatctacatttgggcccctctggctggtacttgcggggtcct |
| | | gctgctttcactcgtgatcactctttactgtaagcgcggtcggaagaagctgctgtacatctttaagcaacc |
| | | cttcatgaggcctgtgcagactactcaagaggaggacggctgttcatgccggttcccagaggaggagg |
| | | aaggcggctgcgaactgcgcgtgaaattcagccgcagcgcagatgctccagcctaccagcaggggc |
| | | agaaccagctctacaacgaactcaatcttggtcggagagaggagtacgacgtgctggacaagcggag |
| | | aggacgggacccagaaatgggcgggaagccgcgcagaaagaatccccaagagggcctgtacaacg |
| | | agctccaaaaggataagatggcagaagcctatagcgagattggtatgaaaggggaacgcagaagagg |
| | | caaaggccacgacggactgtaccagggactcagcaccgccaccaaggacacctatgacgctcttcac |
| | | atgcaggccctgccgcctcgg |
| 357 | VH | CAGGTCCAGCTGCAGGAATCAGGACCAGGGCTGGTGAAACCTAGCG |
| | | AAACTCTGAGTCTGACTTGTACCGTCTCCGGGGTGTCTCTGCCAGACT |
| | | ACGGCGTGAGCTGGATCAGACAGCCCCCTGGCAAGTGCCTGGAGTGG |
| | | ATCGGCGTGATCTGGGGCTCCGAGACCACATACTATCAGAGCTCCCT |
| | | GAAGTCTCGGGTGACCATCTCCAAGGACAACTCTAAGAATCAGGTGA |
| | | GCCTGAAGCTGTCTAGCGTGACCGCCGCCGATACAGCCGTGTACTAT |
| | | TGTGCCAAGCACTACTATTACGGCGGCTCCTATGCCATGGATTACTGG |
| | | GGCCAGGGCACCCTGGTGACAGTGTCCTCT |
| 358 | VH | CAGGTCCAGCTGCAGGAATCCGGCCCAGGACTGGTGAAGCCTAGCGA |
| | | GACCCTGTCCCTGACCTGCACAGTGAGCGGCGTGTCCCTGCCCGATT |
| | | ACGGCGTGAGCTGGATCAGACAGCCCCCTGGCAAGTGTCTGGAGTGG |
| | | ATCGGCGTGATCTGGGGCTCTGAGACCACATACTATCAGTCCTCTCTG |
| | | AAGAGCAGGGTGACCATCTCTAAGGACAACAGCAAGAATCAGGTGT |
| | | CCCTGAAGCTGAGCTCCGTGACCGCAGCAGATACAGCCGTGTACTAT |
| | | TGCGCCAAGCACTACTATTACGGCGGCTCCTATGCTATGGATTATTGG |
| | | GGGCAGGGCACTCTGGTCACTGTCTCATCA |
| 359 | VH | CAGGTGCAGCTGCAGGAATCTGGACCCGGACTGGTGAAACCTAGTGA |
| | | AACTCTGTCTCTGACTTGTACCGTCTCAGGGGTCTCACTGCCAGACTA |
| | | CGGCGTGTCCTGGATCAGACAGCCCCCTGGCAAGTGCCTGGAGTGGA |
| | | TCGGCGTGATCTGGGGCTCTGAGACCACATACTATCAGAGCTCCCTG |
| | | AAGAGCCGGGTGACCATCTCCAAGGACAACTCTAAGAATCAGGTGTC |
| | | CCTGAAGCTGTCTAGCGTGACCGCCGCCGATACAGCCGTGTACTATT |
| | | GTGCCAAGCACTACTATTACGGCGGCAGCTATGCCATGGATTACTGG |
| | | GGCCAGGGCACCCTGGTGACAGTGTCCTCT |
| 360 | VH | CAGGTCCAGCTGCAGGAAAGCGGCCCAGGACTGGTGAAGCCTAGCG |
| | | AGACCCTGTCCCTGACCTGCACAGTGAGCGGCGTGTCCCTGCCTGATT |
| | | ACGGCGTGTCCTGGATCAGACAGCCCCCTGGCAAGTGTCTGGAGTGG |
| | | ATCGGCGTGATCTGGGGCTCCGAGACCACATACTATCAGTCCTCTCTG |
| | | AAGTCTAGGGTGACAATCTCTAAGGACAACAGCAAGAATCAGGTGA |
| | | GCCTGAAGCTGAGCTCCGTGACCGCAGCAGATACAGCCGTGTACTAT |
| | | TGTGCCAAGCACTACTATTACGGCGGCTCTTATGCTATGGATTATTGG |
| | | GGGCAGGGCACTCTGGTCACTGTCTCAAGC |
| 361 | VH | CAGGTGCAGCTGCAGGAGAGCGGCCCAGGACTGGTGAAGCCTTCCG |
| | | AGACACTGTCTCTGACCTGTACAGTGAGCGGCGTGTCCCTGCCCGAC |
| | | TACGGCGTGTCCTGGATCAGACAGCCACCTGGCAAGGGACTGGAGTG |
| | | GATCGGCGTGATCTGGGGCAGCGAGACCACATACTATCAGAGCTCCC |
| | | TGAAGTCCAGGGTGACCATCAGCAAGGACAACTCCAAGAATCAGGT |
| | | GAGCCTGAAGCTGTCTAGCGTGACCGCCGCCGATACAGCCGTGTACT |
| | | ATTGCGCCAAGCACTACTATTACGGCGGCTCCTATGCCATGGATTACT |
| | | GGGGCCAGGGCACCCTGGTCACAGTGTCCTCT |
| 362 | VH | CAGGTGCAGCTGCAGGAGTCTGGCCCAGGACTGGTGAAGCCTTCTGA |
| | | GACCCTGAGCCTGACCTGCACAGTGTCCGGCGTGTCTCTGCCCGATTA |
| | | CGGCGTGTCCTGGATCAGACAGCCACCTGGCAAGGGACTGGAGTGGA |
| | | TCGGCGTGATCTGGGGCTCTGAGACCACATACTATCAGTCTAGCCTG |
| | | AAGAGCCGGGTGACAATCTCCAAGGACAACTCTAAGAATCAGGTGTC |
| | | CCTGAAGCTGTCCTCTGTGACCGCCGCCGATACAGCCGTGTACTATTG |
| | | TGCCAAGCACTACTATTACGGCGGCAGCTATGCCATGGACTACTGGG |
| | | GCCAGGGCACCCTGGTGACAGTGAGCTCC |
| 363 | VH | CAGGTGCAGCTGCAGGAGTCTGGCCCAGGACTGGTGAAGCCTTCTGA |
| | | GACCCTGAGCCTGACCTGCACAGTGAGCGGCGTGTCCCTGCCCGATT |
| | | ACGGCGTGTCCTGGATCAGACAGCCACCTGGCAAGGGACTGGAGTGG |
| | | ATCGGCGTGATCTGGGGCAGCGAGACCACATACTATCAGTCCTCTCT |
| | | GAAGTCCAGGGTGACAATCTCCAAGGACAACTCTAAGAATCAGGTGA |
| | | GCCTGAAGCTGAGCTCCGTGACCGCAGCAGATACAGCCGTGTACTAT |
| | | TGCGCCAAGCACTACTATTACGGCGGCTCCTATGCCATGGACTACTG |
| | | GGGCCAGGGCACCCTGGTCACAGTGTCTAGC |
| 364 | VH | CAGGTGCAGCTGCAGGAGTCCGGCCCAGGACTGGTGAAGCCTTCCGA |
| | | GACACTGTCTCTGACCTGTACAGTGTCCGGCGTGTCTCTGCCCGACTA |
| | | CGGCGTGAGCTGGATCAGACAGCCACCTGGCAAGGGACTGGAGTGG |
| | | ATCGGCGTGATCTGGGGCTCTGAGACCACATACTATCAGTCCTCTCTG |
| | | AAGAGCCGGGTGACCATCAGCAAGGACAACTCCAAGAATCAGGTGT |
| | | CCCTGAAGCTGAGCTCCGTGACCGCAGCAGATACAGCCGTGTACTAT |
| | | TGCGCCAAGCACTACTATTACGGCGGCAGCTATGCCATGGATTACTG |
| | | GGGCCAGGGCACCCTGGTGACAGTGTCTAGC |
| 365 | VL | GAGATCGTGATGACCCAGAGCCCAGCCACACTGAGCCTGTCCCCAGG |
| | | AGAGAGGGCCACACTGTCTTGTAGAGCCAGCCAGGATATCTCCAAGT |
| | | ATCTGAACTGGTACCAGCAGAAGCCTGGACAGGCACCAAGGCTGCTG |
| | | ATCTACCACACCTCTAGACTGCACAGCGGCATCCCTGCCAGGTTTTCT |
| | | GGCAGCGGCTCCGGCACAGACTATACCCTGACAATCTCTAGCCTGCA |
| | | GCCAGAGGATTTCGCCGTGTACTTTTGTCAGCAGGGCAATACTCTGCC |
| | | ATACACCTTTGGATGCGGAACTAAACTGGAAATCAAG |
| 366 | VL | GAAATTGTGATGACCCAGTCCCCCGCTACTCTGTCTCTGTCCCCCGGA |
| | | GAACGGGCTACTCTGTCTTGTCGCGCTTCCCAGGATATTAGCAAGTAC |
| | | CTGAACTGGTATCAGCAGAAGCCAGGACAGGCACCAAGGCTGCTGAT |
| | | CTACCACACCTCTCGCCTGCACAGCGGCATCCCTGCACGGTTCTCTGG |
| | | CAGCGGCTCCGGCACAGACTACACCCTGACAATCAGCTCCCTGCAGC |
| | | CTGAGGATTTCGCCGTGTACTTTTGCCAGCAGGGCAATACCCTGCCAT |
| | | ATACATTTGGCTGTGGCACCAAGCTGGAGATCAAG |
| 367 | VL | GAGATCGTGATGACCCAGTCCCCAGCCACACTGAGCCTGTCCCCAGG |
| | | AGAGAGGGCCACCCTGTCTTGTAGAGCCAGCCAGGATATCTCCAAGT |
| | | ATCTGAACTGGTACCAGCAGAAGCCTGGACAGGCACCAAGGCTGCTG |
| | | ATCTACCACACCTCTAGACTGCACAGCGGCATCCCTGCCAGGTTTTCT |
| | | GGCAGCGGCTCCGGCACAGACTATACCCTGACAATCTCTAGCCTGCA |
| | | GCCAGAGGATTTCGCCGTGTACTTTTGTCAGCAGGGAAATACTCTGCC |
| | | ATACACCTTTGGATGCGGAACTAAACTGGAAATCAAG |
| 368 | VL | GAGATTGTGATGACCCAGTCCCCCGCCACCCTGAGTCTGAGCCCCGG |
| | | AGAACGAGCTACCCTGAGTTGCCGAGCTTCCCAGGACATTTCCAAGT |
| | | ACCTGAACTGGTATCAGCAGAAGCCAGGACAGGCACCAAGGCTGCT |
| | | GATCTACCACACCTCTCGCCTGCACAGCGGCATCCCAGCACGGTTCTC |
| | | TGGCAGCGGCTCCGGCACAGACTACACCCTGACAATCAGCTCCCTGC |
| | | AGCCTGAGGATTTCGCCGTGTACTTTTGCCAGCAGGGCAATACCCTG |
| | | CCATATACATTTGGCTGTGGCACCAAGCTGGAGATCAAG |
| 369 | VL | GAGATCGTGATGACCCAGTCTCCAGCCACACTGTCTCTGAGCCCAGG |
| | | AGAGAGGGCCACCCTGTCTTGCCGCGCCAGCCAGGATATCTCCAAGT |
| | | ATCTGAACTGGTACCAGCAGAAGCCAGGACAGGCACCAAGGCTGCT |
| | | GATCTACCACACCTCTCGCCTGCACAGCGGCATCCCAGCACGGTTCTC |
| | | CGGCTCTGGCAGCGGCACAGACTACACCCTGACAATCTCCTCTCTGC |
| | | AGCCCGAGGATTTCGCCGTGTATTTTTGCCAGCAGGGCAATACCCTG |
| | | CCTTACACATTTGGCCAGGGCACCAAGCTGGAGATCAAG |
| 370 | VL | GAGATCGTGATGACCCAGAGCCCAGCCACACTGAGCCTGTCCCCAGG |
| | | AGAGAGGGCCACCCTGAGCTGCAGAGCCTCCCAGGATATCTCTAAGT |
| | | ATCTGAACTGGTACCAGCAGAAGCCTGGACAGGCACCAAGGCTGCTG |
| | | ATCTACCACACCAGCAGACTGCACTCCGGCATCCCTGCAAGGTTCTCT |
| | | GGCAGCGGCTCCGGCACAGACTACACCCTGACAATCTCTAGCCTGCA |
| | | GCCTGAGGATTTCGCCGTGTATTTTTGTCAGCAGGGCAATACCCTGCC |
| | | ATACACATTTGGCCAGGGCACCAAGCTGGAGATCAAG |
| 371 | VL | GAGATCGTGATGACCCAGAGCCCAGCCACACTGTCTCTGAGCCCAGG |
| | | AGAGAGGGCCACCCTGAGCTGTCGCGCCTCCCAGGATATCTCTAAGT |
| | | ATCTGAACTGGTACCAGCAGAAGCCAGGACAGGCACCAAGGCTGCT |
| | | GATCTACCACACCAGCCGCCTGCACTCCGGCATCCCAGCACGGTTCT |
| | | CCGGCTCTGGCAGCGGCACAGACTACACCCTGACAATCTCCTCTCTG |
| | | CAGCCCGAGGATTTCGCCGTGTATTTTTGCCAGCAGGGCAATACCCT |
| | | GCCTTACACATTTGGCCAGGGCACCAAGCTGGAGATCAAG |
| 372 | VL | GAGATCGTGATGACCCAGTCTCCAGCCACACTGAGCCTGTCCCCAGG |
| | | AGAGAGGGCCACCCTGTCTTGCAGAGCCAGCCAGGATATCTCCAAGT |
| | | ATCTGAACTGGTACCAGCAGAAGCCTGGACAGGCACCAAGGCTGCTG |
| | | ATCTACCACACCTCTAGACTGCACAGCGGCATCCCAGCAAGGTTCTC |
| | | TGGCAGCGGCTCCGGCACAGACTACACCCTGACAATCTCTAGCCTGC |
| | | AGCCTGAGGATTTCGCCGTGTATTTTTGCCAGCAGGGCAATACCCTGC |
| | | CATACACATTTGGCCAGGGCACCAAGCTGGAGATCAAG |
| SEQ ID NO: | Anti-CD19 | QVQLQESGPGLVKPSETLSLTCTVSGVSLPDYGVSWIRQPPGKGLEWIGV |
| 250 | VH | IWGSETTYYQSSLKSRVTISKDNSKNQVSLKLSSVTAADTAVYYCAKHY |
| | | YYGGSYAMDYWGQGTLVTVSS |
| SEQ ID NO: | Anti-CD19 | EIVMTQSPATLSLSPGERATLSCRASQDISKYLNWYQQKPGQAPRLLIYH |
| 251 | VL | TSRLHSGIPARFSGSGSGTDYTLTISSLQPEDFAVYFCQQGNTLPYTFGQG |
| | | TKLEIK |
| SEQ ID NO: | VH | QVQLQESGPGLVKPSETLSLTCTVSGVSLPDYGVSWIRQPPGKCLEWIGV |
| 331 | | IWGSETTYYQSSLKSRVTISKDNSKNQVSLKLSSVTAADTAVYYCAKHY |
| | | YYGGSYAMDYWGQGTLVTVSS |
| SEQ ID NO: | VL | EIVMTQSPATLSLSPGERATLSCRASQDISKYLNWYQQKPGQAPRLLIYH |
| 332 | | TSRLHSGIPARFSGSGSGTDYTLTISSLQPEDFAVYFCQQGNTLPYTFGCG |
| | | TKLEIK |
| 373 | CAR 1 | EIVMTQSPATLSLSPGERATLSCRASQDISKYLNWYQQKPGQAPRLLIYHTSRL |
| | scFv | HSGIPARFSGSGSGTDYTLTISSLQPEDFAVYFCQQGNTLPYTFGQGTKLEIKG |
| | | GGGSGGGGSGGGGSQVQLQESGPGLVKPSETLSLTCTVSGVSLPDYGVSWIRQP |
| | | PGKGLEWIGVIWGSETTYYSSSLKSRVTISKDNSKNQVSLKLSSVTAADTAVYY |
| | | CAKHYYYGGSYAMDYWGQGTLVTVSS |
| 374 | CAR 3 | qvqlqesgpglvkpsetlsltctvsgvslpdygvswirqppgkglewigviwgs |
| | scFv | ettyyssslksrvtiskdnsknqvslklssvtaadtavyycakhyyyggsyamd |
| | | ywgqgtlvtvssggggsggggsggggseivmtqspatlslspgeratlscrasq |
| | | diskylnwyqqkpgqaprlliyhtsrlhsgiparfsgsgsgtdytltisslqpe |
| | | dfavyfcqqgntlpytfgqgtkleik |
| 375 | CAR 4 | qvqlqesgpglvkpsetlsltctvsgvslpdygvswirqppgkglewigviwgs |
| | scFv | ettyyqsslksrvtiskdnsknqvslklssvtaadtavyycakhyyyggsyamd |
| | | ywgqgtlvtvssggggsggggggggseivmtqspatlslspgeratlscrasq |
| | | diskylnwyqqkpgqaprlliyhtsrlhsgiparfsgsgsgtdytltisslqpe |
| | | dfavyfcqqgntlpytfgqgtkleik |
| 376 | CAR 5 | eivmtqspatlslspgeratlscrasqdiskylnwyqqkpgqaprlliyhtsrl |
| | scFv | hsgiparfsgsgsgtdytltisslqpedfavyfcqqgntlpytfgqgtkleikg |
| | | gggsggggsggggsggggsqvqlqesgpglvkpsetlsltctvsgvslpdygvs |
| | | wirqppgkglewigviwgsettyyssslksrvtiskdnsknqvslklssvtaad |
| | | tavyycakhyyyggsyamdywgqgtlvtvss |
| 377 | CAR 6 | eivmtqspatlslspgeratlscrasqdiskylnwyqqkpgqaprlliyhtsrl |
| | scFv | hsgiparfsgsgsgtdytltisslqpedfavyfcqqgntlpytfgqgtkleikg |
| | | gggsggggggggsggggsqvqlqesgpglvkpsetlsltctvsgvslpdygvs |
| | | wirqppgkglewigviwgsettyyqsslksrvtiskdnsknqvslklssvtaad |
| | | tavyycakhyyyggsyamdywgqgtlvtvss |
| 378 | CAR 7 | qvqlqesgpglvkpsetlsltctvsgvslpdygvswirqppgkglewigviwgs |
| | scFv | ettyyssslksrvtiskdnsknqvslklssvtaadtavyycakhyyyggsyamd |
| | | ywgqgtlvtvssggggsggggggggsggggseivmtqspatlslspgeratls |
| | | crasqdiskylnwyqqkpgqaprlliyhtsrlhsgiparfsgsgsgtdytltis |
| | | slqpedfavyfcqqgntlpytfgqgtkleik |
| 379 | CAR 8 | qvqlqesgpglvkpsetlsltctvsgvslpdygvswirqppgkglewigviwgs |
| | scFv | ettyyqsslksrvtiskdnsknqvslklssvtaadtavyycakhyyyggsyamd |
| | | ywgqgtlvtvssggggsggggsggggsggggseivmtqspatlslspgeratls |
| | | crasqdiskylnwyqqkpgqaprlliyhtsrlhsgiparfsgsgsgtdytltis |
| | | slqpedfavyfcqqgntlpytfgqgtkleik |
| 380 | CAR 9 | eivmtqspatlslspgeratlscrasqdiskylnwyqqkpgqaprlliyhtsrl |
| | scFv | hsgiparfsgsgsgtdytltisslqpedfavyfcqqgntlpytfgqgtkleikg |
| | | gggsggggsggggsggggsqvqlqesgpglvkpsetlsltctvsgvslpdygvs |
| | | wirqppgkglewigviwgsettyynsslksrvtiskdnsknqvslklssvtaad |
| | | tavyycakhyyyggsyamdywgqgtlvtvss |
| 381 | CAR 10 | qvqlqesgpglvkpsetlsltctvsgvslpdygvswirqppgkglewigviwgs |
| | scFv | ettyynsslksrvtiskdnsknqvslklssvtaadtavyycakhyyyggsyamd |
| | | ywgqgtlvtvssggggggggsggggsggggseivmtqspatlslspgeratls |
| | | crasqdiskylnwyqqkpgqaprlliyhtsrlhsgiparfsgsgsgtdytltis |
| | | slqpedfavyfcqqgntlpytfgqgtkleik |
| 382 | CAR 11 | eivmtqspatlslspgeratlscrasqdiskylnwyqqkpgqaprlliyhtsrl |
| | scFv | hsgiparfsgsgsgtdytltisslqpedfavyfcqqgntlpytfgqgtkleikg |
| | | gggsggggsggggsqvqlqesgpglvkpsetlsltctvsgvslpdygvswirqp |
| | | pgkglewigviwgsettyynsslksrvtiskdnsknqvslklssvtaadtavyy |
| | | cakhyyyggsyamdywgqgtlvtvss |
| 383 | CAR 12 | qvqlqesgpglvkpsetlsltctvsgvslpdygvswirqppgkglewigviwgs |
| | scFv | ettyynsslksrvtiskdnsknqvslklssvtaadtavyycakhyyyggsyamd |
| | | ywgqgtlvtvssggggsggggsggggseivmtqspatlslspgeratlscrasq |
| | | diskylnwyqqkpgqaprlliyhtsrlhsgiparfsgsgsgtdytltisslqpe |
| | | dfavyfcqqgntlpytfgqgtkleik |
| 384 | CAR A full | atgcttctcctggtgacaagccttctgctctgtgagttaccacacccagcattc |
| | nucleotide | ctcctgatcccagacatccagatgacacagactacatcctccctgtctgcctct |
| | sequence; | ctgggagacagagtcaccatcagttgcagggcaagtcaggacattagtaaatat |
| | with leader | ttaaattggtatcagcagaaaccagatggaactgttaaactcctgatctaccat |
| | | acatcaagattacactcaggagtcccatcaaggttcagtggcagtgggtctgga |
| | | acagattattctctcaccattagcaacctggagcaagaagatattgccacttac |
| | | ttttgccaacagggtaatacgcttccgtacacgttcggaggggggactaagttg |
| | | gaaataacaggctccacctctggatccggcaagcccggatctggcgagggatcc |
| | | accaagggcgaggtgaaactgcaggagtcaggacctggcctggtggcgccctca |
| | | cagagcctgtccgtcacatgcactgtctcaggggtctcattacccgactatggt |
| | | gtaagctggattcgccagcctccacgaaagggtctggagtggctgggagtaata |
| | | tggggtagtgaaaccacatactataattcagctctcaaatccagactgaccatc |
| | | atcaaggacaactccaagagccaagttttcttaaaaatgaacagtctgcaaact |
| | | gatgacacagccatttactactgtgccaaacattattactacggtggtagctat |
| | | gctatggactactggggtcaaggaacctcagtcaccgtctcctcagcggccgca |
| | | attgaagttatgtatcctcctccttacctagacaatgagaagagcaatggaacc |
| | | attatccatgtgaaagggaaacacctttgtccaagtcccctatttcccggacct |
| | | tctaagcccttttgggtgctggtggtggttgggggagtcctggcttgctatagc |
| | | ttgctagtaacagtggcctttattattttctgggtgaggagtaagaggagcagg |
| | | ctcctgcacagtgactacatgaacatgactccccgccgccccgggcccacccgc |
| | | aagcattaccagccctatgccccaccacgcgacttcgcagcctatcgctccaga |
| | | gtgaagttcagcaggagcgcagacgcccccgcgtaccagcagggccagaaccag |
| | | ctctataacgagctcaatctaggacgaagagaggagtacgatgttttggacaag |
| | | agacgtggccgggaccctgagatggggggaaagccgagaaggaagaaccctcag |
| | | gaaggcctgtacaatgaactgcagaaagataagatggcggaggcctacagtgag |
| | | attgggatgaaaggcgagcgccggaggggcaaggggcacgatggcctttaccag |
| | | ggtctcagtacagccaccaaggacacctacgacgcccttcacatgcaggccctg |
| | | ccccctcgc |
| 385 | CAR A- | MLLLVTSLLLCELPHPAFLLIPDIQMTQTTSSLSASLGDRVTISCRASQDISKY |
| | full amino | LNWYQQKPDGTVKLLIYHTSRLHSGVPSRFSGSGSGTDYSLTISNLEQEDIATY |
| | acid | FCQQGNTLPYTFGGGTKLEITGSTSGSGKPGSGEGSTKGEVKLQESGPGLVAPS |
| | transgene | QSLSVTCTVSGVSLPDYGVSWIRQPPRKGLEWLGVIWGSETTYYNSALKSRLTI |
| | sequence; | IKDNSKSQVFLKMNSLQTDDTAIYYCAKHYYYGGSYAMDYWGQGTSVTVSSAAA |
| | with leader | IEVMYPPPYLDNEKSNGTIIHVKGKHLCPSPLFPGPSKPFWVLVVVGGVLACYS |
| | | LLVTVAFIIFWVRSKRSRLLHSDYMNMTPRRPGPTRKHYQPYAPPRDFAAYRSR |
| | | VKFSRSADAPAYQQGQNQLYNELNLGRREEYDVLDKRRGRDPEMGGKPRRKNPQ |
| | | EGLYNELQKDKMAEAYSEIGMKGERRRGKGHDGLYQGLSTATKDTYDALHMQAL |
| | | PPR |
| 386 | CAR A- | atgcttctcctggtgacaagccttctgctctgtgagttaccacacccagcattc |
| | CD19 scFv | ctcctgatcccagacatccagatgacacagactacatcctccctgtctgcctct |
| | nucleotide | ctgggagacagagtcaccatcagttgcagggcaagtcaggacattagtaaatat |
| | sequence | ttaaattggtatcagcagaaaccagatggaactgttaaactcctgatctaccat |
| | with leader | acatcaagattacactcaggagtcccatcaaggttcagtggcagtgggtctgga |
| | | acagattattctctcaccattagcaacctggagcaagaagatattgccacttac |
| | | ttttgccaacagggtaatacgcttccgtacacgttcggaggggggactaagttg |
| | | gaaataacaggctccacctctggatccggcaagcccggatctggcgagggatcc |
| | | accaagggcgaggtgaaactgcaggagtcaggacctggcctggtggcgccctca |
| | | cagagcctgtccgtcacatgcactgtctcaggggtctcattacccgactatggt |
| | | gtaagctggattcgccagcctccacgaaagggtctggagtggctgggagtaata |
| | | tggggtagtgaaaccacatactataattcagctctcaaatccagactgaccatc |
| | | atcaaggacaactccaagagccaagttttcttaaaaatgaacagtctgcaaact |
| | | gatgacacagccatttactactgtgccaaacattattactacggtggtagctat |
| | | gctatggactactggggtcaaggaacctcagtcaccgtctcctca |
| 387 | CAR A- | MLLLVTSLLLCELPHPAFLLIPDIQMTQTTSSLSASLGDRVTISCRASQDISKY |
| | CD19 scFv | LNWYQQKPDGTVKLLIYHTSRLHSGVPSRFSGSGSGTDYSLTISNLEQEDIATY |
| | amino acid | FCQQGNTLPYTFGGGTKLEITGSTSGSGKPGSGEGSTKGEVKLQESGPGLVAPS |
| | sequence; | QSLSVTCTVSGVSLPDYGVSWIRQPPRKGLEWLGVIWGSETTYYNSALKSRLTI |
| | with leader | IKDNSKSQVFLKMNSLQTDDTAIYYCAKHYYYGGSYAMDYWGQGTSVTVS |
| 388 | CAR A- | gacatccagatgacacagactacatcctccctgtctgcctctctgggagacaga |
| | full | gtcaccatcagttgcagggcaagtcaggacattagtaaatatttaaattggtat |
| | nucleotide | cagcagaaaccagatggaactgttaaactcctgatctaccatacatcaagatta |
| | sequence; | cactcaggagtcccatcaaggttcagtggcagtgggtctggaacagattattct |
| | no leader | ctcaccattagcaacctggagcaagaagatattgccacttacttttgccaacag |
| | | ggtaatacgcttccgtacacgttcggaggggggactaagttggaaataacaggc |
| | | tccacctctggatccggcaagcccggatctggcgagggatccaccaagggcgag |
| | | gtgaaactgcaggagtcaggacctggcctggtggcgccctcacagagcctgtcc |
| | | gtcacatgcactgtctcaggggtctcattacccgactatggtgtaagctggatt |
| | | cgccagcctccacgaaagggtctggagtggctgggagtaatatggggtagtgaa |
| | | accacatactataattcagctctcaaatccagactgaccatcatcaaggacaac |
| | | tccaagagccaagttttcttaaaaatgaacagtctgcaaactgatgacacagcc |
| | | atttactactgtgccaaacattattactacggtggtagctatgctatggactac |
| | | tggggtcaaggaacctcagtcaccgtctcctcagcggccgcaattgaagttatg |
| | | tatcctcctccttacctagacaatgagaagagcaatggaaccattatccatgtg |
| | | aaagggaaacacctttgtccaagtcccctatttcccggaccttctaagcccttt |
| | | tgggtgctggtggtggttgggggagtcctggcttgctatagcttgctagtaaca |
| | | gtggcctttattattttctgggtgaggagtaagaggagcaggctcctgcacagt |
| | | gactacatgaacatgactccccgccgccccgggcccacccgcaagcattaccag |
| | | ccctatgccccaccacgcgacttcgcagcctatcgctccagagtgaagttcagc |
| | | aggagcgcagacgcccccgcgtaccagcagggccagaaccagctctataacgag |
| | | ctcaatctaggacgaagagaggagtacgatgttttggacaagagacgtggccgg |
| | | gaccctgagatggggggaaagccgagaaggaagaaccctcaggaaggcctgtac |
| | | aatgaactgcagaaagataagatggcggaggcctacagtgagattgggatgaaa |
| | | ggcgagcgccggaggggcaaggggcacgatggcctttaccagggtctcagtaca |
| | | gccaccaaggacacctacgacgcccttcacatgcaggccctgccccctcgc |
| 389 | CAR A- | DIQMTQTTSSLSASLGDRVTISCRASQDISKYLNWYQQKPDGTVKLLIYHTSRL |
| | full amino | HSGVPSRFSGSGSGTDYSLTISNLEQEDIATYFCQQGNTLPYTFGGGTKLEITG |
| | acid | STSGSGKPGSGEGSTKGEVKLQESGPGLVAPSQSLSVTCTVSGVSLPDYGVSWI |
| | transgene | RQPPRKGLEWLGVIWGSETTYYNSALKSRLTIIKDNSKSQVFLKMNSLQTDDTA |
| | sequence; | IYYCAKHYYYGGSYAMDYWGQGTSVTVSSAAAIEVMYPPPYLDNEKSNGTIIHV |
| | no leader | KGKHLCPSPLFPGPSKPFWVLVVVGGVLACYSLLVTVAFIIFWVRSKRSRLLHS |
| | | DYMNMTPRRPGPTRKHYQPYAPPRDFAAYRSRVKFSRSADAPAYQQGQNQLYNE |
| | | LNLGRREEYDVLDKRRGRDPEMGGKPRRKNPQEGLYNELQKDKMAEAYSEIGMK |
| | | GERRRGKGHDGLYQGLSTATKDTYDALHMQALPPR |
| 390 | CAR A- | gacatccagatgacacagactacatcctccctgtctgcctctctgggagacaga |
| | CD19 scFv | gtcaccatcagttgcagggcaagtcaggacattagtaaatatttaaattggtat |
| | nucleotide; | cagcagaaaccagatggaactgttaaactcctgatctaccatacatcaagatta |
| | no leader | cactcaggagtcccatcaaggttcagtggcagtgggtctggaacagattattct |
| | | ctcaccattagcaacctggagcaagaagatattgccacttacttttgccaacag |
| | | ggtaatacgcttccgtacacgttcggaggggggactaagttggaaataacaggc |
| | | tccacctctggatccggcaagcccggatctggcgagggatccaccaagggcgag |
| | | gtgaaactgcaggagtcaggacctggcctggtggcgccctcacagagcctgtcc |
| | | gtcacatgcactgtctcaggggtctcattacccgactatggtgtaagctggatt |
| | | cgccagcctccacgaaagggtctggagtggctgggagtaatatggggtagtgaa |
| | | accacatactataattcagctctcaaatccagactgaccatcatcaaggacaac |
| | | tccaagagccaagttttcttaaaaatgaacagtctgcaaactgatgacacagcc |
| | | atttactactgtgccaaacattattactacggtggtagctatgctatggactac |
| | | tggggtcaaggaacctcagtcaccgtctcctca |
| 391 | CAR A- | DIQMTQTTSSLSASLGDRVTISCRASQDISKYLNWYQQKPDGTVKLLIYHTSRL |
| | CD19 scFv | HSGVPSRFSGSGSGTDYSLTISNLEQEDIATYFCQQGNTLPYTFGGGTKLEITG |
| | amino acid | STSGSGKPGSGEGSTKGEVKLQESGPGLVAPSQSLSVTCTVSGVSLPDYGVSWI |
| | sequence; | RQPPRKGLEWLGVIWGSETTYYNSALKSRLTIIKDNSKSQVFLKMNSLQTDDTA |
| | no leader | IYYCAKHYYYGGSYAMDYWGQGTSVTVSS |
| 392 | CAR B- | ATGCTGCTGCTGGTGACCAGCCTGCTGCTGTGCGAGCTGCCCCACCCCGCCTTT |
| | full | CTGCTGATCCCCGACATCCAGATGACCCAGACCACCTCCAGCCTGAGCGCCAGC |
| | nucleotide | CTGGGCGACCGGGTGACCATCAGCTGCCGGGCCAGCCAGGACATCAGCAAGTAC |
| | sequence; | CTGAACTGGTATCAGCAGAAGCCCGACGGCACCGTCAAGCTGCTGATCTACCAC |
| | with leader | ACCAGCCGGCTGCACAGCGGCGTGCCCAGCCGGTTTAGCGGCAGCGGCTCCGGC |
| | | ACCGACTACAGCCTGACCATCTCCAACCTGGAACAGGAAGATATCGCCACCTAC |
| | | TTTTGCCAGCAGGGCAACACACTGCCCTACACCTTTGGCGGCGGAACAAAGCTG |
| | | GAAATCACCGGCAGCACCTCCGGCAGCGGCAAGCCTGGCAGCGGCGAGGGCAGC |
| | | ACCAAGGGCGAGGTGAAGCTGCAGGAAAGCGGCCCTGGCCTGGTGGCCCCCAGC |
| | | CAGAGCCTGAGCGTGACCTGCACCGTGAGCGGCGTGAGCCTGCCCGACTACGGC |
| | | GTGAGCTGGATCCGGCAGCCCCCCAGGAAGGGCCTGGAATGGCTGGGCGTGATC |
| | | TGGGGCAGCGAGACCACCTACTACAACAGCGCCCTGAAGAGCCGGCTGACCATC |
| | | ATCAAGGACAACAGCAAGAGCCAGGTGTTCCTGAAGATGAACAGCCTGCAGACC |
| | | GACGACACCGCCATCTACTACTGCGCCAAGCACTACTACTACGGCGGCAGCTAC |
| | | GCCATGGACTACTGGGGCCAGGGCACCAGCGTGACCGTGAGCAGCGAATCTAAG |
| | | TACGGACCGCCCTGCCCCCCTTGCCCTATGTTCTGGGTGCTGGTGGTGGTCGGA |
| | | GGCGTGCTGGCCTGCTACAGCCTGCTGGTCACCGTGGCCTTCATCATCTTTTGG |
| | | GTGAAACGGGGCAGAAAGAAACTCCTGTATATATTCAAACAACCATTTATGAGA |
| | | CCAGTACAAACTACTCAAGAGGAAGATGGCTGTAGCTGCCGATTTCCAGAAGAA |
| | | GAAGAAGGAGGATGTGAACTGCGGGTGAAGTTCAGCAGAAGCGCCGACGCCCCT |
| | | GCCTACCAGCAGGGCCAGAATCAGCTGTACAACGAGCTGAACCTGGGCAGAAGG |
| | | GAAGAGTACGACGTCCTGGATAAGCGGAGAGGCCGGGACCCTGAGATGGGCGGC |
| | | AAGCCTCGGCGGAAGAACCCCCAGGAAGGCCTGTATAACGAACTGCAGAAAGAC |
| | | AAGATGGCCGAGGCCTACAGCGAGATCGGCATGAAGGGCGAGCGGAGGCGGGGC |
| | | AAGGGCCACGACGGCCTGTATCAGGGCCTGTCCACCGCCACCAAGGATACCTAC |
| | | GACGCCCTGCACATGCAGGCCCTGCCCCCAAGG |
| 393 | CAR B- | MLLLVTSLLLCELPHPAFLLIPDIQMTQTTSSLSASLGDRVTISCRASQDISKY |
| | full | LNWYQQKPDGTVKLLIYHTSRLHSGVPSRFSGSGSGTDYSLTISNLEQEDIATY |
| | transgene | FCQQGNTLPYTFGGGTKLEITGSTSGSGKPGSGEGSTKGEVKLQESGPGLVAPS |
| | amino acid | QSLSVTCTVSGVSLPDYGVSWIRQPPRKGLEWLGVIWGSETTYYNSALKSRLTI |
| | sequence; | IKDNSKSQVFLKMNSLQTDDTAIYYCAKHYYYGGSYAMDYWGQGTSVTVSSESK |
| | with leader | YGPPCPPCPMFWVLVVVGGVLACYSLLVTVAFIIFWVKRGRKKLLYIFKQPFMR |
| | | PVQTTQEEDGCSCRFPEEEEGGCELRVKFSRSADAPAYQQGQNQLYNELNLGRR |
| | | EEYDVLDKRRGRDPEMGGKPRRKNPQEGLYNELQKDKMAEAYSEIGMKGERRRG |
| | | KGHDGLYQGLSTATKDTYDALHMQALPPR |
| 394 | CAR B- | ATGCTGCTGCTGGTGACCAGCCTGCTGCTGTGCGAGCTGCCCCACCCCGCCTTT |
| | CD19 scFv | CTGCTGATCCCCGACATCCAGATGACCCAGACCACCTCCAGCCTGAGCGCCAGC |
| | nucleotide | CTGGGCGACCGGGTGACCATCAGCTGCCGGGCCAGCCAGGACATCAGCAAGTAC |
| | sequence; | CTGAACTGGTATCAGCAGAAGCCCGACGGCACCGTCAAGCTGCTGATCTACCAC |
| | with leader | ACCAGCCGGCTGCACAGCGGCGTGCCCAGCCGGTTTAGCGGCAGCGGCTCCGGC |
| | | ACCGACTACAGCCTGACCATCTCCAACCTGGAACAGGAAGATATCGCCACCTAC |
| | | TTTTGCCAGCAGGGCAACACACTGCCCTACACCTTTGGCGGCGGAACAAAGCTG |
| | | GAAATCACCGGCAGCACCTCCGGCAGCGGCAAGCCTGGCAGCGGCGAGGGCAGC |
| | | ACCAAGGGCGAGGTGAAGCTGCAGGAAAGCGGCCCTGGCCTGGTGGCCCCCAGC |
| | | CAGAGCCTGAGCGTGACCTGCACCGTGAGCGGCGTGAGCCTGCCCGACTACGGC |
| | | GTGAGCTGGATCCGGCAGCCCCCCAGGAAGGGCCTGGAATGGCTGGGCGTGATC |
| | | TGGGGCAGCGAGACCACCTACTACAACAGCGCCCTGAAGAGCCGGCTGACCATC |
| | | ATCAAGGACAACAGCAAGAGCCAGGTGTTCCTGAAGATGAACAGCCTGCAGACC |
| | | GACGACACCGCCATCTACTACTGCGCCAAGCACTACTACTACGGCGGCAGCTAC |
| | | GCCATGGACTACTGGGGCCAGGGCACCAGCGTGACCGTGAGCAGC |
| 395 | CAR B- | MLLLVTSLLLCELPHPAFLLIPDIQMTQTTSSLSASLGDRVTISCRASQDISKY |
| | CD19 scFv | LNWYQQKPDGTVKLLIYHTSRLHSGVPSRFSGSGSGTDYSLTISNLEQEDIATY |
| | amino acid | FCQQGNTLPYTFGGGTKLEITGSTSGSGKPGSGEGSTKGEVKLQESGPGLVAPS |
| | sequence; | QSLSVTCTVSGVSLPDYGVSWIRQPPRKGLEWLGVIWGSETTYYNSALKSRLTI |
| | with leader | IKDNSKSQVFLKMNSLQTDDTAIYYCAKHYYYGGSYAMDYWGQGTSVTVSS |
| 396 | CAR B- | GACATCCAGATGACCCAGACCACCTCCAGCCTGAGCGCCAGCCTGGGCGACCGG |
| | full | GTGACCATCAGCTGCCGGGCCAGCCAGGACATCAGCAAGTACCTGAACTGGTAT |
| | nucleotide | CAGCAGAAGCCCGACGGCACCGTCAAGCTGCTGATCTACCACACCAGCCGGCTG |
| | sequence; | CACAGCGGCGTGCCCAGCCGGTTTAGCGGCAGCGGCTCCGGCACCGACTACAGC |
| | no leader | CTGACCATCTCCAACCTGGAACAGGAAGATATCGCCACCTACTTTTGCCAGCAG |
| | | GGCAACACACTGCCCTACACCTTTGGCGGCGGAACAAAGCTGGAAATCACCGGC |
| | | AGCACCTCCGGCAGCGGCAAGCCTGGCAGCGGCGAGGGCAGCACCAAGGGCGAG |
| | | GTGAAGCTGCAGGAAAGCGGCCCTGGCCTGGTGGCCCCCAGCCAGAGCCTGAGC |
| | | GTGACCTGCACCGTGAGCGGCGTGAGCCTGCCCGACTACGGCGTGAGCTGGATC |
| | | CGGCAGCCCCCCAGGAAGGGCCTGGAATGGCTGGGCGTGATCTGGGGCAGCGAG |
| | | ACCACCTACTACAACAGCGCCCTGAAGAGCCGGCTGACCATCATCAAGGACAAC |
| | | AGCAAGAGCCAGGTGTTCCTGAAGATGAACAGCCTGCAGACCGACGACACCGCC |
| | | ATCTACTACTGCGCCAAGCACTACTACTACGGCGGCAGCTACGCCATGGACTAC |
| | | TGGGGCCAGGGCACCAGCGTGACCGTGAGCAGCGAATCTAAGTACGGACCGCCC |
| | | TGCCCCCCTTGCCCTATGTTCTGGGTGCTGGTGGTGGTCGGAGGCGTGCTGGCC |
| | | TGCTACAGCCTGCTGGTCACCGTGGCCTTCATCATCTTTTGGGTGAAACGGGGC |
| | | AGAAAGAAACTCCTGTATATATTCAAACAACCATTTATGAGACCAGTACAAACT |
| | | ACTCAAGAGGAAGATGGCTGTAGCTGCCGATTTCCAGAAGAAGAAGAAGGAGGA |
| | | TGTGAACTGCGGGTGAAGTTCAGCAGAAGCGCCGACGCCCCTGCCTACCAGCAG |
| | | GGCCAGAATCAGCTGTACAACGAGCTGAACCTGGGCAGAAGGGAAGAGTACGAC |
| | | GTCCTGGATAAGCGGAGAGGCCGGGACCCTGAGATGGGCGGCAAGCCTCGGCGG |
| | | AAGAACCCCCAGGAAGGCCTGTATAACGAACTGCAGAAAGACAAGATGGCCGAG |
| | | GCCTACAGCGAGATCGGCATGAAGGGCGAGCGGAGGCGGGGCAAGGGCCACGAC |
| | | GGCCTGTATCAGGGCCTGTCCACCGCCACCAAGGATACCTACGACGCCCTGCAC |
| | | ATGCAGGCCCTGCCCCCAAGG |
| 397 | CAR B- | DIQMTQTTSSLSASLGDRVTISCRASQDISKYLNWYQQKPDGTVKLLIYHTSRL |
| | full amino | HSGVPSRFSGSGSGTDYSLTISNLEQEDIATYFCQQGNTLPYTFGGGTKLEITG |
| | acid | STSGSGKPGSGEGSTKGEVKLQESGPGLVAPSQSLSVTCTVSGVSLPDYGVSWI |
| | transgene | RQPPRKGLEWLGVIWGSETTYYNSALKSRLTIIKDNSKSQVFLKMNSLQTDDTA |
| | sequence; | IYYCAKHYYYGGSYAMDYWGQGTSVTVSSESKYGPPCPPCPMFWVLVVVGGVLA |
| | no leader | CYSLLVTVAFIIFWVKRGRKKLLYIFKQPFMRPVQTTQEEDGCSCRFPEEEEGG |
| | | CELRVKFSRSADAPAYQQGQNQLYNELNLGRREEYDVLDKRRGRDPEMGGKPRR |
| | | KNPQEGLYNELQKDKMAEAYSEIGMKGERRRGKGHDGLYQGLSTATKDTYDALH |
| | | MQALPPR |
| 398 | CAR B- | GACATCCAGATGACCCAGACCACCTCCAGCCTGAGCGCCAGCCTGGGCGACCGG |
| | CD19 scFv | GTGACCATCAGCTGCCGGGCCAGCCAGGACATCAGCAAGTACCTGAACTGGTAT |
| | sequence; | CAGCAGAAGCCCGACGGCACCGTCAAGCTGCTGATCTACCACACCAGCCGGCTG |
| | no leader | CACAGCGGCGTGCCCAGCCGGTTTAGCGGCAGCGGCTCCGGCACCGACTACAGC |
| | | CTGACCATCTCCAACCTGGAACAGGAAGATATCGCCACCTACTTTTGCCAGCAG |
| | | GGCAACACACTGCCCTACACCTTTGGCGGCGGAACAAAGCTGGAAATCACCGGC |
| | | AGCACCTCCGGCAGCGGCAAGCCTGGCAGCGGCGAGGGCAGCACCAAGGGCGAG |
| | | GTGAAGCTGCAGGAAAGCGGCCCTGGCCTGGTGGCCCCCAGCCAGAGCCTGAGC |
| | | GTGACCTGCACCGTGAGCGGCGTGAGCCTGCCCGACTACGGCGTGAGCTGGATC |
| | | CGGCAGCCCCCCAGGAAGGGCCTGGAATGGCTGGGCGTGATCTGGGGCAGCGAG |
| | | ACCACCTACTACAACAGCGCCCTGAAGAGCCGGCTGACCATCATCAAGGACAAC |
| | | AGCAAGAGCCAGGTGTTCCTGAAGATGAACAGCCTGCAGACCGACGACACCGCC |
| | | ATCTACTACTGCGCCAAGCACTACTACTACGGCGGCAGCTACGCCATGGACTAC |
| | | TGGGGCCAGGGCACCAGCGTGACCGTGAGCAGC |
| 399 | CAR B- | DIQMTQTTSSLSASLGDRVTISCRASQDISKYLNWYQQKPDGTVKLLIYHTSRL |
| | CD19 scFv | HSGVPSRFSGSGSGTDYSLTISNLEQEDIATYFCQQGNTLPYTFGGGTKLEITG |
| | sequence; | STSGSGKPGSGEGSTKGEVKLQESGPGLVAPSQSLSVTCTVSGVSLPDYGVSWI |
| | no leader | RQPPRKGLEWLGVIWGSETTYYNSALKSRLTIIKDNSKSQVFLKMNSLQTDDTA |
| | | IYYCAKHYYYGGSYAMDYWGQGTSVTVSS |

### BCMA Car

In some embodiments, the CAR-expressing cell described herein is a BCMA CAR-expressing cell (for example, a cell expressing a CAR that binds to human BCMA). Exemplary BCMA CARs can include sequences disclosed in Table 1 or 16 of WO2016/014565, incorporated herein by reference. The BCMA CAR construct can include an optional leader sequence; an optional hinge domain, for example, a CD8 hinge domain; a transmembrane domain, for example, a CD8 transmembrane domain; an intracellular domain, for example, a 4-1BB intracellular domain; and a functional signaling domain, for example, a CD3 zeta domain. In certain embodiments, the domains are contiguous and in the same reading frame to form a single fusion protein. In other embodiments, the domains are in separate polypeptides, for example, as in an RCAR molecule as described herein.

In some embodiments, the BCMA CAR molecule includes one or more CDRs, VH, VL, scFv, or full-length sequences of BCMA-1, BCMA-2, BCMA-3, BCMA-4, BCMA-5, BCMA-6, BCMA-7, BCMA-8, BCMA-9, BCMA-10, BCMA-11, BCMA-12, BCMA-13, BCMA-14, BCMA-15, 149362, 149363, 149364, 149365, 149366, 149367, 149368, 149369, BCMA_EBB-C1978-A4, BCMA_EBB-C1978-G1, BCMA_EBB-C1979-C1, BCMA_EBB-C1978-C7, BCMA_EBB-C1978-D10, BCMA_EBB-C1979-C12, BCMA_EBB-C1980-G4, BCMA_EBB-C1980-D2, BCMA_EBB-C1978-A10, BCMA_EBB-C1978-D4, BCMA_EBB-C1980-A2, BCMA_EBB-C1981-C3, BCMA_EBB-C1978-G4, A7D12.2, C11D5.3, C12A3.2, or C13F12.1 disclosed in WO2016/014565, or a sequence substantially (for example, 95-99%) identical thereto.

Additional exemplary BCMA-targeting sequences that can be used in the anti-BCMA CAR constructs are disclosed in WO 2017/021450, WO 2017/011804, WO 2017/025038, WO 2016/090327, WO 2016/130598, WO 2016/210293, WO 2016/090320, WO 2016/014789, WO 2016/094304, WO 2016/154055, WO 2015/166073, WO 2015/188119, WO 2015/158671, U.S. Pat. Nos. 9,243,058, 8,920,776, 9,273,141, 7,083,785, 9,034,324, US 2007/0049735, US 2015/0284467, US 2015/0051266, US 2015/0344844, US 2016/0131655, US 2016/0297884, US 2016/0297885, US 2017/0051308, US 2017/0051252, US 2017/0051252, WO 2016/020332, WO 2016/087531, WO 2016/079177, WO 2015/172800, WO 2017/008169, U.S. Pat. No. 9,340,621, US 2013/0273055, US 2016/0176973, US 2015/0368351, US 2017/0051068, US 2016/0368988, and US 2015/0232557, herein incorporated by reference in their entirety. In some embodiments, additional exemplary BCMA CAR constructs are generated using the VH and VL sequences from PCT Publication WO2012/0163805 (the contents of which are hereby incorporated by reference in its entirety).

In some embodiments, BCMA CARs comprise a sequence, for example, a CDR, VH, VL, scFv, or full-CAR sequence, disclosed in Tables 3-15, or a sequence having at least 80%, 85%, 90%, 95%, or 99% identity thereto. In some embodiments, the antigen binding domain comprises a human antibody or a human antibody fragment. In some embodiments, the human anti-BCMA binding domain comprises one or more (for example, all three) LC CDR1, LC CDR2, and LC CDR3 of a human anti-BCMA binding domain described herein (for example, in Tables 3-15), and/or one or more (for example, all three) HC CDR1, HC CDR2, and HC CDR3 of a human anti-BCMA binding domain described herein (for example, in Tables 3-15). In some embodiments, the human anti-BCMA binding domain comprises a human VL described herein (for example, in Tables 3, 7, 11, 11a, and 12) and/or a human VH described herein (for example, in Tables 3, 7, 11, 11a, and 12). In some embodiments, the anti-BCMA binding domain is a scFv comprising a VL and a VH of an amino acid sequence of Tables 3, 7, 11, 11a, and 12. In some embodiments, the anti-BCMA binding domain (for example, an scFv) comprises: a VL comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 3, 7, 11, 11a, and 12, or a sequence with 95-99% identity with an amino acid sequence of Tables 3, 7, 11, 11a, and 12, and/or a VH comprising an amino acid sequence having at least one, two or three modifications (for example, substitutions, for example, conservative substitutions) but not more than 30, 20 or 10 modifications (for example, substitutions, for example, conservative substitutions) of an amino acid sequence provided in Tables 3, 7, 11, 11a, and 12, or a sequence with 95-99% identity to an amino acid sequence of Tables 3, 7, 11, 11a, and 12.

TABLE 3
Amino acid and nucleic acid sequences of
exemplary PALLAS-derived anti-BCMA mlecules

| SEQ ID | Name/ | |
|---|---|---|
| NO | Description | Sequence |
| R1B6 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWVPYDVSWYFDY |
| NO: 46 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWVPYDVSWYFDY |
| NO: 46 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWVPYDVSWYFDY |
| NO: 51 | (IMGT) | |
| SEQ ID | VH | EVQLLESGGGLVQPGGSLRLSCAASGFTFSSYAMSWVRQAPGKGLEWVS |
| NO: 52 | | AISGSGGSTYYADSVKGRFTISRDNSKNTLYLQMNSLRAEDTAVYYCARR |
| | | EWVPYDVSWYFDYWGQGTLVTVSS |
| SEQ ID | DNA VH | GAAGTGCAGTTGCTGGAGTCAGGCGGAGGACTGGTGCAGCCCGGAGG |
| NO: 53 | | ATCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGGTGCCCTACGATGTCAGCTGGTACTTCGACTA |
| | | CTGGGGACAGGGCACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKLLIYAA |
| NO: 61 | | SSLQSGVPSRFSGSGSGTDFTLTISSLQPEDFATYYCQQSYSTPLTFGQGTK |
| | | VEIK |
| SEQ ID | DNA VL | GACATTCAAATGACTCAGTCCCCGTCCTCCCTCTCCGCCTCCGTGGGA |
| NO: 62 | | GATCGCGTCACGATCACGTGCAGGGCCAGCCAGAGCATCTCCAGCTAC |
| | | CTGAACTGGTACCAGCAGAAGCCAGGGAAGGCACCGAAGCTCCTGAT |
| | | CTACGCCGCTAGCTCGCTGCAGTCCGGCGTCCCTTCACGGTTCTCGGG |
| | | ATCGGGCTCAGGCACCGACTTCACCCTGACCATTAGCAGCCTGCAGCC |
| | | GGAGGACTTCGCGACATACTACTGTCAGCAGTCATACTCCACCCCTCT |
| | | GACCTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGLEWVS |
| NO: 64 | linker-VL) | AISGSGGSTYYADSVKGRFTISRDNSKNTLYLQMNSLRAEDTAVYYCARR |
| | | EWVPYDVSWYFDYWGQGTLVTVSSGGGGSGGGGSGGGGSGGGGSDIQ |
| | | MTQSPSSLSASVGDRVTITCRASQSISSYLNWYQQKPGKAPKLLIYAASSL |
| | | QSGVPSRFSGSGSGTDFTLTISSLQPEDFATYYCQQSYSTPLTFGQGTKVEI |
| | | K |
| SEQ ID | DNA scFv | GAAGTGCAGTTGCTGGAGTCAGGCGGAGGACTGGTGCAGCCCGGAGG |
| NO: 65 | | ATCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGGTGCCCTACGATGTCAGCTGGTACTTCGACTA |
| | | CTGGGGACAGGGCACTCTCGTGACTGTGTCCTCCGGTGGTGGTGGATC |
| | | GGGGGGTGGTGGTTCGGGCGGAGGAGGATCTGGAGGAGGAGGGTCGG |
| | | ACATTCAAATGACTCAGTCCCCGTCCTCCCTCTCCGCCTCCGTGGGAG |
| | | ATCGCGTCACGATCACGTGCAGGGCCAGCCAGAGCATCTCCAGCTACC |
| | | TGAACTGGTACCAGCAGAAGCCAGGGAAGGCACCGAAGCTCCTGATC |
| | | TACGCCGCTAGCTCGCTGCAGTCCGGCGTCCCTTCACGGTTCTCGGGA |
| | | TCGGGCTCAGGCACCGACTTCACCCTGACCATTAGCAGCCTGCAGCCG |
| | | GAGGACTTCGCGACATACTACTGTCAGCAGTCATACTCCACCCCTCTG |
| | | ACCTTCGGCCAAGGGACCAAAGTGGAGATCAAG |
| SEQ ID | Full CAR | EVQLLESGGGLVQPGGSLRLSCAASGFTFSSYAMSWVRQAPGKGLEWVS |
| NO: 66 | amino acid | AISGSGGSTYYADSVKGRFTISRDNSKNTLYLQMNSLRAEDTAVYYCARR |
| | sequence | EWVPYDVSWYFDYWGQGTLVTVSSGGGGSGGGGSGGGGSGGGGSDIQ |
| | | MTQSPSSLSASVGDRVTITCRASQSISSYLNWYQQKPGKAPKLLIYAASSL |
| | | QSGVPSRFSGSGSGTDFTLTISSLQPEDFATYYCQQSYSTPLTFGQGTKVEI |
| | | KTTTPAPRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDG |
| | | LYQGLSTATKDTYDALHMQALPPR |
| SEQ ID | Full CAR | GAAGTGCAGTTGCTGGAGTCAGGCGGAGGACTGGTGCAGCCCGGAGG |
| NO: 67 | DNA | ATCGCTTCGCTTGAGCTGCGCAGCCTCAGGCTTTACCTTCTCCTCCTAC |
| | sequence | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGGTGCCCTACGATGTCAGCTGGTACTTCGACTA |
| | | CTGGGGACAGGGCACTCTCGTGACTGTGTCCTCCGGTGGTGGTGGATC |
| | | GGGGGGTGGTGGTTCGGGCGGAGGAGGATCTGGAGGAGGAGGGTCGG |
| | | ACATTCAAATGACTCAGTCCCCGTCCTCCCTCTCCGCCTCCGTGGGAG |
| | | ATCGCGTCACGATCACGTGCAGGGCCAGCCAGAGCATCTCCAGCTACC |
| | | TGAACTGGTACCAGCAGAAGCCAGGGAAGGCACCGAAGCTCCTGATC |
| | | TACGCCGCTAGCTCGCTGCAGTCCGGCGTCCCTTCACGGTTCTCGGGA |
| | | TCGGGCTCAGGCACCGACTTCACCCTGACCATTAGCAGCCTGCAGCCG |
| | | GAGGACTTCGCGACATACTACTGTCAGCAGTCATACTCCACCCCTCTG |
| | | ACCTTCGGCCAAGGGACCAAAGTGGAGATCAAGACCACTACCCCAGC |
| | | ACCGAGGCCACCCACCCCGGCTCCTACCATCGCCTCCCAGCCTCTGTC |
| | | CCTGCGTCCGGAGGCATGTAGACCCGCAGCTGGTGGGGCCGTGCATAC |
| | | CCGGGGTCTTGACTTCGCCTGCGATATCTACATTTGGGCCCCTCTGGCT |
| | | GGTACTTGCGGGGTCCTGCTGCTTTCACTCGTGATCACTCTTTACTGTA |
| | | AGCGCGGTCGGAAGAAGCTGCTGTACATCTTTAAGCAACCCTTCATGA |
| | | GGCCTGTGCAGACTACTCAAGAGGAGGACGGCTGTTCATGCCGGTTCC |
| | | CAGAGGAGGAGGAAGGCGGCTGCGAACTGCGCGTGAAATTCAGCCGC |
| | | AGCGCAGATGCTCCAGCCTACCAGCAGGGGCAGAACCAGCTCTACAA |
| | | CGAACTCAATCTTGGTCGGAGAGAGGAGTACGACGTGCTGGACAAGC |
| | | GGAGAGGACGGGACCCAGAAATGGGCGGGAAGCCGCGCAGAAAGAA |
| | | TCCCCAAGAGGGCCTGTACAACGAGCTCCAAAAGGATAAGATGGCAG |
| | | AAGCCTATAGCGAGATTGGTATGAAAGGGGAACGCAGAAGAGGCAAA |
| | | GGCCACGACGGACTGTACCAGGGACTCAGCACCGCCACCAAGGACAC |
| | | CTATGACGCTCTTCACATGCAGGCCCTGCCGCCTCGG |
| R1F2 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWYDDWYLDY |
| NO: 68 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWYDDWYLDY |
| NO: 68 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWYDDWYLDY |
| NO: 69 | (IMGT) | |
| SEQ ID | VH | EVQLLESGGGLVQPGGSLRLSCAASGFTFSSYAMSWVRQAPGKGLEWVS |
| NO: 70 | | AISGSGGSTYYADSVKGRFTISRDNSKNTLYLQMNSLRAEDTAVYYCARR |
| | | EWWYDDWYLDYWGQGTLVTVSS |
| SEQ ID | DNA VH | GAAGTGCAGTTGCTGGAGTCAGGCGGAGGACTGGTGCAGCCCGGAGG |
| NO: 71 | | ATCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGTGGTACGACGATTGGTACCTGGACTACTGGG |
| | | GACAGGGCACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKLLIYAA |
| NO: 61 | | SSLQSGVPSRFSGSGSGTDFTLTISSLQPEDFATYYCQQSYSTPLTFGQGTK |
| | | VEIK |
| SEQ ID | DNA VL | GACATTCAAATGACTCAGTCCCCGTCCTCCCTCTCCGCCTCCGTGGGA |
| NO: 62 | | GATCGCGTCACGATCACGTGCAGGGCCAGCCAGAGCATCTCCAGCTAC |
| | | CTGAACTGGTACCAGCAGAAGCCAGGGAAGGCACCGAAGCTCCTGAT |
| | | CTACGCCGCTAGCTCGCTGCAGTCCGGCGTCCCTTCACGGTTCTCGGG |
| | | ATCGGGCTCAGGCACCGACTTCACCCTGACCATTAGCAGCCTGCAGCC |
| | | GGAGGACTTCGCGACATACTACTGTCAGCAGTCATACTCCACCCCTCT |
| | | GACCTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGLEWVS |
| NO: 72 | linker-VL) | AISGSGGSTYYADSVKGRFTISRDNSKNTLYLQMNSLRAEDTAVYYCARR |
| | | EWWYDDWYLDYWGQGTLVTVSSGGGGSGGGGSGGGGSGGGGSDIQMT |
| | | QSPSSLSASVGDRVTITCRASQSISSYLNWYQQKPGKAPKLLIYAASSLQS |
| | | GVPSRFSGSGSGTDFTLTISSLQPEDFATYYCQQSYSTPLTFGQGTKVEIK |
| SEQ ID | DNA scFv | GAAGTGCAGTTGCTGGAGTCAGGCGGAGGACTGGTGCAGCCCGGAGG |
| NO: 73 | | ATCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGTGGTACGACGATTGGTACCTGGACTACTGGG |
| | | GACAGGGCACTCTCGTGACTGTGTCCTCCGGTGGTGGTGGATCGGGGG |
| | | GTGGTGGTTCGGGCGGAGGAGGATCTGGAGGAGGAGGGTCGGACATT |
| | | CAAATGACTCAGTCCCCGTCCTCCCTCTCCGCCTCCGTGGGAGATCGC |
| | | GTCACGATCACGTGCAGGGCCAGCCAGAGCATCTCCAGCTACCTGAAC |
| | | TGGTACCAGCAGAAGCCAGGGAAGGCACCGAAGCTCCTGATCTACGC |
| | | CGCTAGCTCGCTGCAGTCCGGCGTCCCTTCACGGTTCTCGGGATCGGG |
| | | CTCAGGCACCGACTTCACCCTGACCATTAGCAGCCTGCAGCCGGAGGA |
| | | CTTCGCGACATACTACTGTCAGCAGTCATACTCCACCCCTCTGACCTTC |
| | | GGCCAAGGGACCAAAGTGGAGATCAAG |
| SEQ ID | Full CAR | EVQLLESGGGLVQPGGSLRLSCAASGFTFSSYAMSWVRQAPGKGLEWVS |
| NO: 74 | amino acid | AISGSGGSTYYADSVKGRFTISRDNSKNTLYLQMNSLRAEDTAVYYCARR |
| | sequence | EWWYDDWYLDYWGQGTLVTVSSGGGGSGGGGSGGGGSGGGGSDIQMT |
| | | QSPSSLSASVGDRVTITCRASQSISSYLNWYQQKPGKAPKLLIYAASSLQS |
| | | GVPSRFSGSGSGTDFTLTISSLQPEDFATYYCQQSYSTPLTFGQGTKVEIKT |
| | | TTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEE |
| | | EEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDGL |
| | | YQGLSTATKDTYDALHMQALPPR |
| SEQ ID | Full CAR | GAAGTGCAGTTGCTGGAGTCAGGCGGAGGACTGGTGCAGCCCGGAGG |
| NO: 75 | DNA | ATCGCTTCGCTTGAGCTGCGCAGCCTCAGGCTTTACCTTCTCCTCCTAC |
| | sequence | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGTGGTACGACGATTGGTACCTGGACTACTGGG |
| | | GACAGGGCACTCTCGTGACTGTGTCCTCCGGTGGTGGTGGATCGGGGG |
| | | GTGGTGGTTCGGGCGGAGGAGGATCTGGAGGAGGAGGGTCGGACATT |
| | | CAAATGACTCAGTCCCCGTCCTCCCTCTCCGCCTCCGTGGGAGATCGC |
| | | GTCACGATCACGTGCAGGGCCAGCCAGAGCATCTCCAGCTACCTGAAC |
| | | TGGTACCAGCAGAAGCCAGGGAAGGCACCGAAGCTCCTGATCTACGC |
| | | CGCTAGCTCGCTGCAGTCCGGCGTCCCTTCACGGTTCTCGGGATCGGG |
| | | CTCAGGCACCGACTTCACCCTGACCATTAGCAGCCTGCAGCCGGAGGA |
| | | CTTCGCGACATACTACTGTCAGCAGTCATACTCCACCCCTCTGACCTTC |
| | | GGCCAAGGGACCAAAGTGGAGATCAAGACCACTACCCCAGCACCGAG |
| | | GCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGG |
| | | TCTTGACTTCGCCTGCGATATCTACATTTGGGCCCCTCTGGCTGGTACT |
| | | TGCGGGGTCCTGCTGCTTTCACTCGTGATCACTCTTTACTGTAAGCGCG |
| | | GTCGGAAGAAGCTGCTGTACATCTTTAAGCAACCCTTCATGAGGCCTG |
| | | TGCAGACTACTCAAGAGGAGGACGGCTGTTCATGCCGGTTCCCAGAGG |
| | | AGGAGGAAGGCGGCTGCGAACTGCGCGTGAAATTCAGCCGCAGCGCA |
| | | GATGCTCCAGCCTACCAGCAGGGGCAGAACCAGCTCTACAACGAACT |
| | | CAATCTTGGTCGGAGAGAGGAGTACGACGTGCTGGACAAGCGGAGAG |
| | | GACGGGACCCAGAAATGGGCGGGAAGCCGCGCAGAAAGAATCCCCAA |
| | | GAGGGCCTGTACAACGAGCTCCAAAAGGATAAGATGGCAGAAGCCTA |
| | | TAGCGAGATTGGTATGAAAGGGGAACGCAGAAGAGGCAAAGGCCACG |
| | | ACGGACTGTACCAGGGACTCAGCACCGCCACCAAGGACACCTATGAC |
| | | GCTCTTCACATGCAGGCCCTGCCGCCTCGG |
| R1G5 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWGESWLFDY |
| NO: 76 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWGESWLFDY |
| NO: 76 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWGESWLFDY |
| NO: 77 | (IMGT) | |
| SEQ ID | VH | EVQLLESGGGLVQPGGSLRLSCAASGFTFSSYAMSWVRQAPGKGLEWVS |
| NO: 78 | | AISGSGGSTYYADSVKGRFTISRDNSKNTLYLQMNSLRAEDTAVYYCARR |
| | | EWWGESWLFDYWGQGTLVTVSS |
| SEQ ID | DNA VH | GAAGTGCAGTTGCTGGAGTCAGGCGGAGGACTGGTGCAGCCCGGAGG |
| NO: 79 | | ATCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGTGGGGAGAAAGCTGGCTGTTCGACTACTGGG |
| | | GACAGGGCACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKLLIYAA |
| NO: 61 | | SSLQSGVPSRFSGSGSGTDFTLTISSLQPEDFATYYCQQSYSTPLTFGQGTK |
| | | VEIK |
| SEQ ID | DNA VL | GACATTCAAATGACTCAGTCCCCGTCCTCCCTCTCCGCCTCCGTGGGA |
| NO: 62 | | GATCGCGTCACGATCACGTGCAGGGCCAGCCAGAGCATCTCCAGCTAC |
| | | CTGAACTGGTACCAGCAGAAGCCAGGGAAGGCACCGAAGCTCCTGAT |
| | | CTACGCCGCTAGCTCGCTGCAGTCCGGCGTCCCTTCACGGTTCTCGGG |
| | | ATCGGGCTCAGGCACCGACTTCACCCTGACCATTAGCAGCCTGCAGCC |
| | | GGAGGACTTCGCGACATACTACTGTCAGCAGTCATACTCCACCCCTCT |
| | | GACCTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGLEWVS |
| NO: 80 | linker-VL) | AISGSGGSTYYADSVKGRFTISRDNSKNTLYLQMNSLRAEDTAVYYCARR |
| | | EWWGESWLFDYWGQGTLVTVSSGGGGSGGGGSGGGGSGGGGSDIQMT |
| | | QSPSSLSASVGDRVTITCRASQSISSYLNWYQQKPGKAPKLLIYAASSLQS |
| | | GVPSRFSGSGSGTDFTLTISSLQPEDFATYYCQQSYSTPLTFGQGTKVEIK |
| SEQ ID | DNA scFv | GAAGTGCAGTTGCTGGAGTCAGGCGGAGGACTGGTGCAGCCCGGAGG |
| NO: 81 | | ATCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGTGGGGAGAAAGCTGGCTGTTCGACTACTGGG |
| | | GACAGGGCACTCTCGTGACTGTGTCCTCCGGTGGTGGTGGATCGGGGG |
| | | GTGGTGGTTCGGGCGGAGGAGGATCTGGAGGAGGAGGGTCGGACATT |
| | | CAAATGACTCAGTCCCCGTCCTCCCTCTCCGCCTCCGTGGGAGATCGC |
| | | GTCACGATCACGTGCAGGGCCAGCCAGAGCATCTCCAGCTACCTGAAC |
| | | TGGTACCAGCAGAAGCCAGGGAAGGCACCGAAGCTCCTGATCTACGC |
| | | CGCTAGCTCGCTGCAGTCCGGCGTCCCTTCACGGTTCTCGGGATCGGG |
| | | CTCAGGCACCGACTTCACCCTGACCATTAGCAGCCTGCAGCCGGAGGA |
| | | CTTCGCGACATACTACTGTCAGCAGTCATACTCCACCCCTCTGACCTTC |
| | | GGCCAAGGGACCAAAGTGGAGATCAAG |
| SEQ ID | Full CAR | EVQLLESGGGLVQPGGSLRLSCAASGFTFSSYAMSWVRQAPGKGLEWVS |
| NO: 82 | amino acid | AISGSGGSTYYADSVKGRFTISRDNSKNTLYLQMNSLRAEDTAVYYCARR |
| | sequence | EWWGESWLFDYWGQGTLVTVSSGGGGSGGGGSGGGGSGGGGSDIQMT |
| | | QSPSSLSASVGDRVTITCRASQSISSYLNWYQQKPGKAPKLLIYAASSLOS |
| | | GVPSRFSGSGSGTDFTLTISSLQPEDFATYYCQQSYSTPLTFGQGTKVEIKT |
| | | TTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEE |
| | | EEGGCELRVKFSRSADAPAYQQGQNQLYNELNLGRREEYDVLDKRRGR |
| | | DPEMGGKPRRKNPQEGLYNELQKDKMAEAYSEIGMKGERRRGKGHDGL |
| | | YQGLSTATKDTYDALHMQALPPR |
| SEQ ID | Full CAR | GAAGTGCAGTTGCTGGAGTCAGGCGGAGGACTGGTGCAGCCCGGAGG |
| NO: 83 | DNA | ATCGCTTCGCTTGAGCTGCGCAGCCTCAGGCTTTACCTTCTCCTCCTAC |
| | sequence | GCCATGTCCTGGGTCAGACAGGCTCCCGGGAAGGGACTGGAATGGGT |
| | | GTCCGCCATTAGCGGTTCCGGCGGAAGCACTTACTATGCCGACTCTGT |
| | | GAAGGGCCGCTTCACTATCTCCCGGGACAACTCCAAGAACACCCTGTA |
| | | TCTCCAAATGAATTCCCTGAGGGCCGAAGATACCGCGGTGTACTACTG |
| | | CGCTAGACGGGAGTGGTGGGGAGAAAGCTGGCTGTTCGACTACTGGG |
| | | GACAGGGCACTCTCGTGACTGTGTCCTCCGGTGGTGGTGGATCGGGGG |
| | | GTGGTGGTTCGGGCGGAGGAGGATCTGGAGGAGGAGGGTCGGACATT |
| | | CAAATGACTCAGTCCCCGTCCTCCCTCTCCGCCTCCGTGGGAGATCGC |
| | | GTCACGATCACGTGCAGGGCCAGCCAGAGCATCTCCAGCTACCTGAAC |
| | | TGGTACCAGCAGAAGCCAGGGAAGGCACCGAAGCTCCTGATCTACGC |
| | | CGCTAGCTCGCTGCAGTCCGGCGTCCCTTCACGGTTCTCGGGATCGGG |
| | | CTCAGGCACCGACTTCACCCTGACCATTAGCAGCCTGCAGCCGGAGGA |
| | | CTTCGCGACATACTACTGTCAGCAGTCATACTCCACCCCTCTGACCTTC |
| | | GGCCAAGGGACCAAAGTGGAGATCAAGACCACTACCCCAGCACCGAG |
| | | GCCACCCACCCCGGCTCCTACCATCGCCTCCCAGCCTCTGTCCCTGCGT |
| | | CCGGAGGCATGTAGACCCGCAGCTGGTGGGGCCGTGCATACCCGGGG |
| | | TCTTGACTTCGCCTGCGATATCTACATTTGGGCCCCTCTGGCTGGTACT |
| | | TGCGGGGTCCTGCTGCTTTCACTCGTGATCACTCTTTACTGTAAGCGCG |
| | | GTCGGAAGAAGCTGCTGTACATCTTTAAGCAACCCTTCATGAGGCCTG |
| | | TGCAGACTACTCAAGAGGAGGACGGCTGTTCATGCCGGTTCCCAGAGG |
| | | AGGAGGAAGGCGGCTGCGAACTGCGCGTGAAATTCAGCCGCAGCGCA |
| | | GATGCTCCAGCCTACCAGCAGGGGCAGAACCAGCTCTACAACGAACT |
| | | CAATCTTGGTCGGAGAGAGGAGTACGACGTGCTGGACAAGCGGAGAG |
| | | GACGGGACCCAGAAATGGGCGGGAAGCCGCGCAGAAAGAATCCCCAA |
| | | GAGGGCCTGTACAACGAGCTCCAAAAGGATAAGATGGCAGAAGCCTA |
| | | TAGCGAGATTGGTATGAAAGGGGAACGCAGAAGAGGCAAAGGCCACG |
| | | ACGGACTGTACCAGGGACTCAGCACCGCCACCAAGGACACCTATGAC |
| | | GCTCTTCACATGCAGGCCCTGCCGCCTCGG |

TABLE 4
Kabat CDRs of exemplary PALLAS-derived anti-BCMA molecules

| Kabat | HCDR1 | HCDR2 | HCDR3 | LCDR1 | LCDR2 | LCDR3 |
|---|---|---|---|---|---|---|
| R1B6 | SYAMS | AISGSGGSTY | REWVPYDVS | RASQSISS | AASSL | QQSYSTP |
| | (SEQ ID | YADSVKG | WYFDY (SEQ | YLN (SEQ | QS | LT (SEQ |
| | NO: 44) | (SEQ ID NO: | ID NO: 46) | ID NO: 54) | (SEQ ID | ID NO: |
| | | 45) | | | NO: 55) | 56) |
| R1F2 | SYAMS | AISGSGGSTY | REWWYDD | RASQSISS | AASSL | QQSYSTP |
| | (SEQ ID | YADSVKG | WYLDY (SEQ | YLN (SEQ | QS | LT (SEQ |
| | NO: 44) | (SEQ ID NO: | ID NO: 68) | ID NO: 54) | (SEQ ID | ID NO: |
| | | 45) | | | NO: 55) | 56) |
| R1G5 | SYAMS | AISGSGGSTY | REWWGESW | RASQSISS | AASSL | QQSYSTP |
| | (SEQ ID | YADSVKG | LFDY (SEQ | YLN (SEQ | QS | LT (SEQ |
| | NO: 44) | (SEQ ID NO: | ID NO: 76) | ID NO: 54) | (SEQ ID | ID NO: |
| | | 45) | | | NO: 55) | 56) |
| Consensus | SYAMS | AISGSGGSTY | REWX₁X₂X₃X₄ | RASQSISS | AASSL | QQSYSTP |
| | (SEQ ID | YADSVKG | X₅X₆WX₇X₈D | YLN (SEQ | QS | LT (SEQ |
| | NO: 44) | (SEQ ID NO: | Y, wherein X₁ | ID NO: 54) | (SEQ ID | ID NO: |
| | | 45) | is absent or V; | | NO: 55) | 56) |
| | | | X₂ is absent or | | | |
| | | | P; X₃ is W or | | | |
| | | | Y; X₄ is G, Y, | | | |
| | | | or D; X₅ is E, | | | |
| | | | D, or V; X₆ is | | | |
| | | | S or D; X₇ is L | | | |
| | | | or Y; and X₈ is | | | |
| | | | For L (SEQ | | | |
| | | | ID NO: 84) | | | |

TABLE 5
Chothia CDRs of exemplary PALLAS-derived anti-BCMA molecules

| Chothia | HCDR1 | HCDR2 | HCDR3 | LCDR1 | LCDR2 | LCDR3 |
|---|---|---|---|---|---|---|
| R1B6 | GFTFSSY | SGSGGS (SEQ | REWVPYDVS | SQSISSY | AAS | SYSTPL |
| | (SEQ ID | ID NO: 48) | WYFDY (SEQ | (SEQ ID | (SEQ ID | (SEQ ID |
| | NO: 47) | | ID NO: 46) | NO: 57) | NO: 58) | NO: 59) |
| R1F2 | GFTFSSY | SGSGGS (SEQ | REWWYDD | SQSISSY | AAS | SYSTPL |
| | (SEQ ID | ID NO: 48) | WYLDY (SEQ | (SEQ ID | (SEQ ID | (SEQ ID |
| | NO: 47) | | ID NO: 68) | NO: 57) | NO: 58) | NO: 59) |
| R1G5 | GFTFSSY | SGSGGS (SEQ | REWWGESW | SQSISSY | AAS | SYSTPL |
| | (SEQ ID | ID NO: 48) | LFDY (SEQ | (SEQ ID | (SEQ ID | (SEQ ID |
| | NO: 47) | | ID NO: 76) | NO: 57) | NO: 58) | NO: 59) |
| Consensus | GFTFSSY | SGSGGS (SEQ | REWX₁X₂X₃X₄ | SQSISSY | AAS | SYSTPL |
| | (SEQ ID | ID NO: 48) | X₅X₆WX₇X₈D | (SEQ ID | (SEQ ID | (SEQ ID |
| | NO: 47) | | Y, wherein X₁ | NO: 57) | NO: 58) | NO: 59) |
| | | | is absent or V; | | | |
| | | | X₂ is absent or | | | |
| | | | P; X₃ is W or | | | |
| | | | Y; X₄ is G, Y, | | | |
| | | | or D; X₅ is E, | | | |
| | | | D, or V; X₆ is | | | |
| | | | S or D; X₇ is L | | | |
| | | | or Y; and X₈ is | | | |
| | | | For L (SEQ | | | |
| | | | ID NO: 84) | | | |

TABLE 6
IMGT CDRs of exemplary PALLAS-derived anti-BCMA molecules

| IMGT | HCDR1 | HCDR2 | HCDR3 | LCDR1 | LCDR2 | LCDR3 |
|---|---|---|---|---|---|---|
| R1B6 | GFTFSSYA | ISGSGGST | ARREWVPY | QSISSY | AAS | QQSYSTP |
| | (SEQ ID | (SEQ ID NO: | DVSWYFDY | (SEQ ID | (SEQ ID | LT (SEQ |
| | NO: 49) | 50) | (SEQ ID NO: | NO: 60) | NO: 58) | ID NO: |
| | | | 51) | | | 56) |
| R1F2 | GFTFSSYA | ISGSGGST | ARREWWYD | QSISSY | AAS | QQSYSTP |
| | (SEQ ID | (SEQ ID NO: | DWYLDY | (SEQ ID | (SEQ ID | LT (SEQ |
| | NO: 49) | 50) | (SEQ ID NO: | NO: 60) | NO: 58) | ID NO: |
| | | | 69) | | | 56) |
| R1G5 | GFTFSSYA | ISGSGGST | ARREWWGE | QSISSY | AAS | QQSYSTP |
| | (SEQ ID | (SEQ ID NO: | SWLFDY | (SEQ ID | (SEQ ID | LT (SEQ |
| | NO: 49) | 50) | (SEQ ID NO: | NO: 60) | NO: 58) | ID NO: |
| | | | 77) | | | 56) |
| Consensus | GFTFSSYA | ISGSGGST | ARREWX₁X₂ | QSISSY | AAS | QQSYSTP |
| | (SEQ ID | (SEQ ID NO: | X₃X₄X₅X₆WX₇ | (SEQ ID | (SEQ ID | LT (SEQ |
| | NO: 49) | 50) | X₈DY, wherein | NO: 60) | NO: 58) | ID NO: |
| | | | X₁ is absent or | | | 56) |
| | | | V; X₂ is absent | | | |
| | | | or P; X₃ is W | | | |
| | | | or Y; X₄ is G, | | | |
| | | | Y, or D; X₅ is | | | |
| | | | E, D, or V; X₆ | | | |
| | | | is S or D; X₇ is | | | |
| | | | L or Y; and X₈ | | | |
| | | | is For L (SEQ | | | |
| | | | ID NO: 85) | | | |

TABLE 7
Amino acid and nucleic acid sequences
of exemplary B cell-derived anti-BCMA molecules

| SEQ ID | Name/ | |
|---|---|---|
| NO | Description | Sequence |
| PI61 | | |
| SEQ ID | HCDR1 | SYGMH |
| NO: 86 | (Kabat) | |
| SEQ ID | HCDR2 | VISYDGSNKYYADSVKG |
| NO: 87 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYDGSN |
| NO: 89 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYDGSNK |
| NO: 91 | (IMGT) | |
| SEQ ID | HCDR3 | GGSGYALHDDYYGLDV |
| NO: 92 | (IMGT) | |
| SEQ ID | VH | QVQLQESGGGVVQPGRSLRLSCAASGFTFSSYGMHWVRQAPGKGLEWVA |
| NO: 93 | | VISYDGSNKYYADSVKGRFTISRDNSKNTLYLQMNSLRAEDTAVYYCGGS |
| | | GYALHDDYYGLDVWGQGTLVTVSS |
| SEQ ID | DNA VH | CAAGTGCAGCTGCAGGAATCCGGTGGCGGAGTCGTGCAGCCTGGAAGG |
| NO: 94 | | AGCCTGAGACTCTCATGCGCCGCGTCAGGGTTCACCTTTTCCTCCTACG |
| | | GGATGCATTGGGTCAGACAGGCCCCCGGAAAGGGACTCGAATGGGTGG |
| | | CTGTGATCAGCTACGACGGCTCCAACAAGTACTACGCCGACTCCGTGA |
| | | AAGGCCGGTTCACTATCTCCCGGGACAACTCCAAGAACACGCTGTATCT |
| | | GCAAATGAATTCACTGCGCGCGGAGGATACCGCTGTGTACTACTGCGG |
| | | TGGCTCCGGTTACGCCCTGCACGATGACTATTACGGCCTTGACGTCTGG |
| | | GGCCAGGGAACCCTCGTGACTGTGTCCAGC |
| SEQ ID | LCDR1 | TGTSSDVGGYNYVS |
| NO: 95 | (Kabat) | |
| SEQ ID | LCDR2 | DVSNRPS |
| NO: 96 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | DVS |
| NO: 99 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | DVS |
| NO: 99 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHPGKAPKLMIY |
| NO: 102 | | DVSNRPSGVSNRFSGSKSGNTASLTISGLQAEDEADYYCSSYTSSSTLYVFG |
| | | SGTKVTVL |
| SEQ ID | DNA VL | CAGAGCGCACTGACTCAGCCGGCATCCGTGTCCGGTAGCCCCGGACAG |
| NO: 103 | | TCGATTACCATCTCCTGTACCGGCACCTCCTCCGACGTGGGAGGGTACA |
| | | ACTACGTGTCGTGGTACCAGCAGCACCCAGGAAAGGCCCCTAAGTTGA |
| | | TGATCTACGATGTGTCAAACCGCCCGTCTGGAGTCTCCAACCGGTTCTC |
| | | CGGCTCCAAGTCCGGCAACACCGCCAGCCTGACCATTAGCGGGCTGCA |
| | | AGCCGAGGATGAGGCCGACTACTACTGCTCGAGCTACACATCCTCGAG |
| | | CACCCTCTACGTGTTCGGCTCGGGGACTAAGGTCACCGTGCTG |
| SEQ ID | Linker | GGGGSGGGGSGGGGS |
| NO: 104 | | |
| SEQ ID | scFv (VH- | QVQLQESGGGVVQPGRSLRLSCAASGFTFSSYGMHWVRQAPGKGLEWVA |
| NO: 105 | linker-VL) | VISYDGSNKYYADSVKGRFTISRDNSKNTLYLQMNSLRAEDTAVYYCGGS |
| | | GYALHDDYYGLDVWGQGTLVTVSSGGGGSGGGGSGGGGSQSALTQPAS |
| | | VSGSPGQSITISCTGTSSDVGGYNYVSWYQQHPGKAPKLMIYDVSNRPSGV |
| | | SNRFSGSKSGNTASLTISGLQAEDEADYYCSSYTSSSTLYVFGSGTKVTVL |
| SEQ ID | DNA scFv | CAAGTGCAGCTGCAGGAATCCGGTGGCGGAGTCGTGCAGCCTGGAAGG |
| NO: 106 | | AGCCTGAGACTCTCATGCGCCGCGTCAGGGTTCACCTTTTCCTCCTACG |
| | | GGATGCATTGGGTCAGACAGGCCCCCGGAAAGGGACTCGAATGGGTGG |
| | | CTGTGATCAGCTACGACGGCTCCAACAAGTACTACGCCGACTCCGTGA |
| | | AAGGCCGGTTCACTATCTCCCGGGACAACTCCAAGAACACGCTGTATCT |
| | | GCAAATGAATTCACTGCGCGCGGAGGATACCGCTGTGTACTACTGCGG |
| | | TGGCTCCGGTTACGCCCTGCACGATGACTATTACGGCCTTGACGTCTGG |
| | | GGCCAGGGAACCCTCGTGACTGTGTCCAGCGGTGGAGGAGGTTCGGGC |
| | | GGAGGAGGATCAGGAGGGGGTGGATCGCAGAGCGCACTGACTCAGCC |
| | | GGCATCCGTGTCCGGTAGCCCCGGACAGTCGATTACCATCTCCTGTACC |
| | | GGCACCTCCTCCGACGTGGGAGGGTACAACTACGTGTCGTGGTACCAG |
| | | CAGCACCCAGGAAAGGCCCCTAAGTTGATGATCTACGATGTGTCAAAC |
| | | CGCCCGTCTGGAGTCTCCAACCGGTTCTCCGGCTCCAAGTCCGGCAACA |
| | | CCGCCAGCCTGACCATTAGCGGGCTGCAAGCCGAGGATGAGGCCGACT |
| | | ACTACTGCTCGAGCTACACATCCTCGAGCACCCTCTACGTGTTCGGCTC |
| | | GGGGACTAAGGTCACCGTGCTG |
| SEQ ID | Full CAR | QVQLQESGGGVVQPGRSLRLSCAASGFTFSSYGMHWVRQAPGKGLEWVA |
| NO: 107 | amino acid | VISYDGSNKYYADSVKGRFTISRDNSKNTLYLQMNSLRAEDTAVYYCGGS |
| | sequence | GYALHDDYYGLDVWGQGTLVTVSSGGGGSGGGGSGGGGSQSALTQPAS |
| | | VSGSPGQSITISCTGTSSDVGGYNYVSWYQQHPGKAPKLMIYDVSNRPSGV |
| | | SNRFSGSKSGNTASLTISGLQAEDEADYYCSSYTSSSTLYVFGSGTKVTVLT |
| | | TTPAPRPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCRFPEEE |
| | | EGGCELRVKFSRSADAPAYQQGQNQLYNELNLGRREEYDVLDKRRGRDP |
| | | EMGGKPRRKNPQEGLYNELQKDKMAEAYSEIGMKGERRRGKGHDGLYQ |
| | | GLSTATKDTYDALHMQALPPR |
| SEQ ID | Full CAR | CAAGTGCAGCTGCAGGAATCCGGTGGCGGAGTCGTGCAGCCTGGAAGG |
| NO: 108 | DNA | AGCCTGAGACTCTCATGCGCCGCGTCAGGGTTCACCTTTTCCTCCTACG |
| | sequence | GGATGCATTGGGTCAGACAGGCCCCCGGAAAGGGACTCGAATGGGTGG |
| | | CTGTGATCAGCTACGACGGCTCCAACAAGTACTACGCCGACTCCGTGA |
| | | AAGGCCGGTTCACTATCTCCCGGGACAACTCCAAGAACACGCTGTATCT |
| | | GCAAATGAATTCACTGCGCGCGGAGGATACCGCTGTGTACTACTGCGG |
| | | TGGCTCCGGTTACGCCCTGCACGATGACTATTACGGCCTTGACGTCTGG |
| | | GGCCAGGGAACCCTCGTGACTGTGTCCAGCGGTGGAGGAGGTTCGGGC |
| | | GGAGGAGGATCAGGAGGGGGTGGATCGCAGAGCGCACTGACTCAGCC |
| | | GGCATCCGTGTCCGGTAGCCCCGGACAGTCGATTACCATCTCCTGTACC |
| | | GGCACCTCCTCCGACGTGGGAGGGTACAACTACGTGTCGTGGTACCAG |
| | | CAGCACCCAGGAAAGGCCCCTAAGTTGATGATCTACGATGTGTCAAAC |
| | | CGCCCGTCTGGAGTCTCCAACCGGTTCTCCGGCTCCAAGTCCGGCAACA |
| | | CCGCCAGCCTGACCATTAGCGGGCTGCAAGCCGAGGATGAGGCCGACT |
| | | ACTACTGCTCGAGCTACACATCCTCGAGCACCCTCTACGTGTTCGGCTC |
| | | GGGGACTAAGGTCACCGTGCTGACCACTACCCCAGCACCGAGGCCACC |
| | | CACCCCGGCTCCTACCATCGCCTCCCAGCCTCTGTCCCTGCGTCCGGAG |
| | | GCATGTAGACCCGCAGCTGGTGGGGCCGTGCATACCCGGGGTCTTGAC |
| | | TTCGCCTGCGATATCTACATTTGGGCCCCTCTGGCTGGTACTTGCGGGG |
| | | TCCTGCTGCTTTCACTCGTGATCACTCTTTACTGTAAGCGCGGTCGGAA |
| | | GAAGCTGCTGTACATCTTTAAGCAACCCTTCATGAGGCCTGTGCAGACT |
| | | ACTCAAGAGGAGGACGGCTGTTCATGCCGGTTCCCAGAGGAGGAGGAA |
| | | GGCGGCTGCGAACTGCGCGTGAAATTCAGCCGCAGCGCAGATGCTCCA |
| | | GCCTACCAGCAGGGGCAGAACCAGCTCTACAACGAACTCAATCTTGGT |
| | | CGGAGAGAGGAGTACGACGTGCTGGACAAGCGGAGAGGACGGGACCC |
| | | AGAAATGGGCGGGAAGCCGCGCAGAAAGAATCCCCAAGAGGGCCTGT |
| | | ACAACGAGCTCCAAAAGGATAAGATGGCAGAAGCCTATAGCGAGATTG |
| | | GTATGAAAGGGGAACGCAGAAGAGGCAAAGGCCACGACGGACTGTAC |
| | | CAGGGACTCAGCACCGCCACCAAGGACACCTATGACGCTCTTCACATG |
| | | CAGGCCCTGCCGCCTCGG |
| B61-02 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAPGKGLEWVA |
| NO: 112 | | VISYKGSNKYYADSVKGRFTISRDNSKNTLYLQMNSLRAEDTAVYYCGGS |
| | | GYALHDDYYGLDVWGQGTLVTVSS |
| SEQ ID | DNA VH | CAAGTGCAGCTTGTCGAATCGGGAGGCGGAGTGGTGCAGCCTGGACGA |
| NO: 113 | | TCGCTCCGGCTCTCATGTGCCGCGAGCGGATTCACCTTCTCGAGCTACG |
| | | GCATGCACTGGGTCAGACAAGCCCCAGGAAAGGGCCTGGAATGGGTGG |
| | | CTGTCATCTCGTACAAGGGCTCAAACAAGTACTACGCCGACTCCGTGAA |
| | | GGGCCGGTTCACCATCTCCCGCGATAACTCCAAGAATACCCTCTATCTG |
| | | CAAATGAACAGCCTGAGGGCCGAGGATACTGCAGTGTACTACTGCGGG |
| | | GGTTCAGGCTACGCGCTGCACGACGACTACTACGGATTGGACGTCTGG |
| | | GGCCAAGGAACTCTTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSALYV |
| NO: 115 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSALY |
| NO: 117 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSALYV |
| NO: 115 | (IMGT) | |
| SEQ ID | VL | QSALTQPASVSGSPGQSITISCTGTSSDVGGYNYVSWYQQHPGKAPKLMIY |
| NO: 118 | | EVSNRLRGVSNRFSGSKSGNTASLTISGLQAEDEADYYCSSYTSSSALYVF |
| | | GSGTKVTVL |
| SEQ ID | DNA VL | CAGAGCGCGCTGACTCAGCCTGCCTCCGTGAGCGGTTCGCCGGGACAG |
| NO: 119 | | TCCATTACCATTTCGTGCACCGGGACCTCCTCCGACGTGGGAGGCTACA |
| | | ACTACGTGTCCTGGTACCAGCAGCATCCCGGAAAGGCCCCGAAGCTGA |
| | | TGATCTACGAAGTGTCGAACAGACTGCGGGGAGTCTCCAACCGCTTTTC |
| | | CGGGTCCAAGTCCGGCAACACCGCCAGCCTGACCATCAGCGGGCTCCA |
| | | GGCAGAAGATGAGGCTGACTATTACTGCTCCTCCTACACGTCAAGCTCC |
| | | GCCCTCTACGTGTTCGGGTCCGGGACCAAAGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAPGKGLEWVA |
| NO: 120 | linker-VL) | VISYKGSNKYYADSVKGRFTISRDNSKNTLYLQMNSLRAEDTAVYYCGGS |
| | | GYALHDDYYGLDVWGQGTLVTVSSGGGGSGGGGSGGGGSGGGGSQSAL |
| | | TQPASVSGSPGQSITISCTGTSSDVGGYNYVSWYQQHPGKAPKLMIYEVSN |
| | | RLRGVSNRFSGSKSGNTASLTISGLQAEDEADYYCSSYTSSSALYVFGSGT |
| | | KVTVL |
| SEQ ID | DNA scFv | CAAGTGCAGCTTGTCGAATCGGGAGGCGGAGTGGTGCAGCCTGGACGA |
| NO: 121 | | TCGCTCCGGCTCTCATGTGCCGCGAGCGGATTCACCTTCTCGAGCTACG |
| | | GCATGCACTGGGTCAGACAAGCCCCAGGAAAGGGCCTGGAATGGGTGG |
| | | CTGTCATCTCGTACAAGGGCTCAAACAAGTACTACGCCGACTCCGTGAA |
| | | GGGCCGGTTCACCATCTCCCGCGATAACTCCAAGAATACCCTCTATCTG |
| | | CAAATGAACAGCCTGAGGGCCGAGGATACTGCAGTGTACTACTGCGGG |
| | | GGTTCAGGCTACGCGCTGCACGACGACTACTACGGATTGGACGTCTGG |
| | | GGCCAAGGAACTCTTGTGACCGTGTCCTCTGGTGGAGGCGGATCAGGG |
| | | GGTGGCGGATCTGGGGGTGGTGGTTCCGGGGGAGGAGGATCGCAGAGC |
| | | GCGCTGACTCAGCCTGCCTCCGTGAGCGGTTCGCCGGGACAGTCCATTA |
| | | CCATTTCGTGCACCGGGACCTCCTCCGACGTGGGAGGCTACAACTACGT |
| | | GTCCTGGTACCAGCAGCATCCCGGAAAGGCCCCGAAGCTGATGATCTA |
| | | CGAAGTGTCGAACAGACTGCGGGGAGTCTCCAACCGCTTTTCCGGGTCC |
| | | AAGTCCGGCAACACCGCCAGCCTGACCATCAGCGGGCTCCAGGCAGAA |
| | | GATGAGGCTGACTATTACTGCTCCTCCTACACGTCAAGCTCCGCCCTCT |
| | | ACGTGTTCGGGTCCGGGACCAAAGTCACTGTGCTG |
| SEQ ID | Full CAR | QVQLVESGGGVVQPGRSLRLSCAASGFTFSSYGMHWVRQAPGKGLEWVA |
| NO: 122 | amino acid | VISYKGSNKYYADSVKGRFTISRDNSKNTLYLQMNSLRAEDTAVYYCGGS |
| | sequence | GYALHDDYYGLDVWGQGTLVTVSSGGGGSGGGGSGGGGSGGGGSQSAL |
| | | TQPASVSGSPGQSITISCTGTSSDVGGYNYVSWYQQHPGKAPKLMIYEVSN |
| | | RLRGVSNRFSGSKSGNTASLTISGLQAEDEADYYCSSYTSSSALYVFGSGT |
| | | KVTVLTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVLDKR |
| | | RGRDPEMGGKPRRKNPQEGLYNELQKDKMAEAYSEIGMKGERRRGKGH |
| | | DGLYQGLSTATKDTYDALHMQALPPR |
| SEQ ID | Full CAR | CAAGTGCAGCTTGTCGAATCGGGAGGCGGAGTGGTGCAGCCTGGACGA |
| NO: 123 | DNA | TCGCTCCGGCTCTCATGTGCCGCGAGCGGATTCACCTTCTCGAGCTACG |
| | sequence | GCATGCACTGGGTCAGACAAGCCCCAGGAAAGGGCCTGGAATGGGTGG |
| | | CTGTCATCTCGTACAAGGGCTCAAACAAGTACTACGCCGACTCCGTGAA |
| | | GGGCCGGTTCACCATCTCCCGCGATAACTCCAAGAATACCCTCTATCTG |
| | | CAAATGAACAGCCTGAGGGCCGAGGATACTGCAGTGTACTACTGCGGG |
| | | GGTTCAGGCTACGCGCTGCACGACGACTACTACGGATTGGACGTCTGG |
| | | GGCCAAGGAACTCTTGTGACCGTGTCCTCTGGTGGAGGCGGATCAGGG |
| | | GGTGGCGGATCTGGGGGTGGTGGTTCCGGGGGAGGAGGATCGCAGAGC |
| | | GCGCTGACTCAGCCTGCCTCCGTGAGCGGTTCGCCGGGACAGTCCATTA |
| | | CCATTTCGTGCACCGGGACCTCCTCCGACGTGGGAGGCTACAACTACGT |
| | | GTCCTGGTACCAGCAGCATCCCGGAAAGGCCCCGAAGCTGATGATCTA |
| | | CGAAGTGTCGAACAGACTGCGGGGAGTCTCCAACCGCTTTTCCGGGTCC |
| | | AAGTCCGGCAACACCGCCAGCCTGACCATCAGCGGGCTCCAGGCAGAA |
| | | GATGAGGCTGACTATTACTGCTCCTCCTACACGTCAAGCTCCGCCCTCT |
| | | ACGTGTTCGGGTCCGGGACCAAAGTCACTGTGCTGACCACTACCCCAGC |
| | | ACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCTG |
| | | GTACTTGCGGGGTCCTGCTGCTTTCACTCGTGATCACTCTTTACTGTAAG |
| | | CGCGGTCGGAAGAAGCTGCTGTACATCTTTAAGCAACCCTTCATGAGGC |
| | | CTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGC |
| | | GCAGATGCTCCAGCCTACCAGCAGGGGCAGAACCAGCTCTACAACGAA |
| | | CTCAATCTTGGTCGGAGAGAGGAGTACGACGTGCTGGACAAGCGGAGA |
| | | GGACGGGACCCAGAAATGGGCGGGAAGCCGCGCAGAAAGAATCCCCA |
| | | AGAGGGCCTGTACAACGAGCTCCAAAAGGATAAGATGGCAGAAGCCTA |
| | | TAGCGAGATTGGTATGAAAGGGGAACGCAGAAGAGGCAAAGGCCACG |
| | | ACGGACTGTACCAGGGACTCAGCACCGCCACCAAGGACACCTATGACG |
| | | CTCTTCACATGCAGGCCCTGCCGCCTCGG |
| B61-10 | | |
| SEQ ID | HCDR1 | SYGMH |
| NO: 86 | (Kabat) | |
| SEQ ID | HCDR2 | VISYKGSNKYYADSVKG |
| NO: 109 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYKGSN |
| NO: 110 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYKGSNK |
| NO: 111 | (IMGT) | |
| SEQ ID | HCDR3 | GGSGYALHDDYYGLDV |
| NO: 92 | (IMGT) | |
| SEQ ID | VH | QVQLVESGGGVVQPGRSLRLSCAASGFTFSSYGMHWVRQAPGKGLEWVA |
| NO: 112 | | VISYKGSNKYYADSVKGRFTISRDNSKNTLYLQMNSLRAEDTAVYYCGGS |
| | | GYALHDDYYGLDVWGQGTLVTVSS |
| SEQ ID | DNA VH | CAAGTGCAGCTTGTCGAATCGGGAGGCGGAGTGGTGCAGCCTGGACGA |
| NO: 113 | | TCGCTCCGGCTCTCATGTGCCGCGAGCGGATTCACCTTCTCGAGCTACG |
| | | GCATGCACTGGGTCAGACAAGCCCCAGGAAAGGGCCTGGAATGGGTGG |
| | | CTGTCATCTCGTACAAGGGCTCAAACAAGTACTACGCCGACTCCGTGAA |
| | | GGGCCGGTTCACCATCTCCCGCGATAACTCCAAGAATACCCTCTATCTG |
| | | CAAATGAACAGCCTGAGGGCCGAGGATACTGCAGTGTACTACTGCGGG |
| | | GGTTCAGGCTACGCGCTGCACGACGACTACTACGGATTGGACGTCTGG |
| | | GGCCAAGGAACTCTTGTGACCGTGTCCTCT |
| SEQ ID | LCDR1 | TGTSSDVGGYNYVS |
| NO: 95 | (Kabat) | |
| SEQ ID | LCDR2 | EVSNRLR |
| NO: 114 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | EVS |
| NO: 116 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | EVS |
| NO: 116 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHPGKAPKLMIY |
| NO: 124 | | EVSNRLRGVSNRFSGSKSGNTASLTISGLQAEDEADYYCSSYTSSSTLYVFG |
| | | SGTKVTVL |
| SEQ ID | DNA VL | CAGAGCGCGCTGACTCAGCCTGCCTCCGTGAGCGGTTCGCCGGGACAG |
| NO: 125 | | TCCATTACCATTTCGTGCACCGGGACCTCCTCCGACGTGGGAGGCTACA |
| | | ACTACGTGTCCTGGTACCAGCAGCATCCCGGAAAGGCCCCGAAGCTGA |
| | | TGATCTACGAAGTGTCGAACAGACTGCGGGGAGTCTCCAACCGCTTTTC |
| | | CGGGTCCAAGTCCGGCAACACCGCCAGCCTGACCATCAGCGGGCTCCA |
| | | GGCAGAAGATGAGGCTGACTATTACTGCTCCTCCTACACGTCAAGCTCC |
| | | ACCCTCTACGTGTTCGGGTCCGGGACCAAAGTCACTGTGCTG |
| SEQ ID | Linker | GGGGSGGGGSGGGGSGGGGS |
| NO: 63 | | |
| SEQ ID | scFv (VH- | QVQLVESGGGVVQPGRSLRLSCAASGFTFSSYGMHWVRQAPGKGLEWVA |
| NO: 126 | linker-VL) | VISYKGSNKYYADSVKGRFTISRDNSKNTLYLQMNSLRAEDTAVYYCGGS |
| | | GYALHDDYYGLDVWGQGTLVTVSSGGGGSGGGGSGGGGSGGGGSQSAL |
| | | TQPASVSGSPGQSITISCTGTSSDVGGYNYVSWYQQHPGKAPKLMIYEVSN |
| | | RLRGVSNRFSGSKSGNTASLTISGLQAEDEADYYCSSYTSSSTLYVFGSGTK |
| | | VTVL |
| SEQ ID | DNA scFv | CAAGTGCAGCTTGTCGAATCGGGAGGCGGAGTGGTGCAGCCTGGACGA |
| NO: 127 | | TCGCTCCGGCTCTCATGTGCCGCGAGCGGATTCACCTTCTCGAGCTACG |
| | | GCATGCACTGGGTCAGACAAGCCCCAGGAAAGGGCCTGGAATGGGTGG |
| | | CTGTCATCTCGTACAAGGGCTCAAACAAGTACTACGCCGACTCCGTGAA |
| | | GGGCCGGTTCACCATCTCCCGCGATAACTCCAAGAATACCCTCTATCTG |
| | | CAAATGAACAGCCTGAGGGCCGAGGATACTGCAGTGTACTACTGCGGG |
| | | GGTTCAGGCTACGCGCTGCACGACGACTACTACGGATTGGACGTCTGG |
| | | GGCCAAGGAACTCTTGTGACCGTGTCCTCTGGTGGAGGCGGATCAGGG |
| | | GGTGGCGGATCTGGGGGTGGTGGTTCCGGGGGAGGAGGATCGCAGAGC |
| | | GCGCTGACTCAGCCTGCCTCCGTGAGCGGTTCGCCGGGACAGTCCATTA |
| | | CCATTTCGTGCACCGGGACCTCCTCCGACGTGGGAGGCTACAACTACGT |
| | | GTCCTGGTACCAGCAGCATCCCGGAAAGGCCCCGAAGCTGATGATCTA |
| | | CGAAGTGTCGAACAGACTGCGGGGAGTCTCCAACCGCTTTTCCGGGTCC |
| | | AAGTCCGGCAACACCGCCAGCCTGACCATCAGCGGGCTCCAGGCAGAA |
| | | GATGAGGCTGACTATTACTGCTCCTCCTACACGTCAAGCTCCACCCTCT |
| | | ACGTGTTCGGGTCCGGGACCAAAGTCACTGTGCTG |
| SEQ ID | Full CAR | QVQLVESGGGVVQPGRSLRLSCAASGFTFSSYGMHWVRQAPGKGLEWVA |
| NO: 128 | amino acid | VISYKGSNKYYADSVKGRFTISRDNSKNTLYLQMNSLRAEDTAVYYCGGS |
| | sequence | GYALHDDYYGLDVWGQGTLVTVSSGGGGSGGGGSGGGGSGGGGSQSAL |
| | | TQPASVSGSPGQSITISCTGTSSDVGGYNYVSWYQQHPGKAPKLMIYEVSN |
| | | RLRGVSNRFSGSKSGNTASLTISGLQAEDEADYYCSSYTSSSTLYVFGSGTK |
| | | VTVLTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCSC |
| | | RFPEEEEGGCELRVKFSRSADAPAYQQGQNQLYNELNLGRREEYDVLDKR |
| | | RGRDPEMGGKPRRKNPQEGLYNELQKDKMAEAYSEIGMKGERRRGKGH |
| | | DGLYQGLSTATKDTYDALHMQALPPR |
| SEQ ID | Full CAR | CAAGTGCAGCTTGTCGAATCGGGAGGCGGAGTGGTGCAGCCTGGACGA |
| NO: 129 | DNA | TCGCTCCGGCTCTCATGTGCCGCGAGCGGATTCACCTTCTCGAGCTACG |
| | sequence | GCATGCACTGGGTCAGACAAGCCCCAGGAAAGGGCCTGGAATGGGTGG |
| | | CTGTCATCTCGTACAAGGGCTCAAACAAGTACTACGCCGACTCCGTGAA |
| | | GGGCCGGTTCACCATCTCCCGCGATAACTCCAAGAATACCCTCTATCTG |
| | | CAAATGAACAGCCTGAGGGCCGAGGATACTGCAGTGTACTACTGCGGG |
| | | GGTTCAGGCTACGCGCTGCACGACGACTACTACGGATTGGACGTCTGG |
| | | GGCCAAGGAACTCTTGTGACCGTGTCCTCTGGTGGAGGCGGATCAGGG |
| | | GGTGGCGGATCTGGGGGTGGTGGTTCCGGGGGAGGAGGATCGCAGAGC |
| | | GCGCTGACTCAGCCTGCCTCCGTGAGCGGTTCGCCGGGACAGTCCATTA |
| | | CCATTTCGTGCACCGGGACCTCCTCCGACGTGGGAGGCTACAACTACGT |
| | | GTCCTGGTACCAGCAGCATCCCGGAAAGGCCCCGAAGCTGATGATCTA |
| | | CGAAGTGTCGAACAGACTGCGGGGAGTCTCCAACCGCTTTTCCGGGTCC |
| | | AAGTCCGGCAACACCGCCAGCCTGACCATCAGCGGGCTCCAGGCAGAA |
| | | GATGAGGCTGACTATTACTGCTCCTCCTACACGTCAAGCTCCACCCTCT |
| | | ACGTGTTCGGGTCCGGGACCAAAGTCACTGTGCTGACCACTACCCCAGC |
| | | ACCGAGGCCACCCACCCCGGCTCCTACCATCGCCTCCCAGCCTCTGTCC |
| | | CTGCGTCCGGAGGCATGTAGACCCGCAGCTGGTGGGGCCGTGCATACC |
| | | CGGGGTCTTGACTTCGCCTGCGATATCTACATTTGGGCCCCTCTGGCTG |
| | | GTACTTGCGGGGTCCTGCTGCTTTCACTCGTGATCACTCTTTACTGTAAG |
| | | CGCGGTCGGAAGAAGCTGCTGTACATCTTTAAGCAACCCTTCATGAGGC |
| | | CTGTGCAGACTACTCAAGAGGAGGACGGCTGTTCATGCCGGTTCCCAG |
| | | AGGAGGAGGAAGGCGGCTGCGAACTGCGCGTGAAATTCAGCCGCAGC |
| | | GCAGATGCTCCAGCCTACCAGCAGGGGCAGAACCAGCTCTACAACGAA |
| | | CTCAATCTTGGTCGGAGAGAGGAGTACGACGTGCTGGACAAGCGGAGA |
| | | GGACGGGACCCAGAAATGGGCGGGAAGCCGCGCAGAAAGAATCCCCA |
| | | AGAGGGCCTGTACAACGAGCTCCAAAAGGATAAGATGGCAGAAGCCTA |
| | | TAGCGAGATTGGTATGAAAGGGGAACGCAGAAGAGGCAAAGGCCACG |
| | | ACGGACTGTACCAGGGACTCAGCACCGCCACCAAGGACACCTATGACG |
| | | CTCTTCACATGCAGGCCCTGCCGCCTCGG |

TABLE 8
Kabat CDRs of exemplary B cell-derived anti-BCMA molecules

| Kabat | HCDR1 | HCDR2 | HCDR3 | LCDR1 | LCDR2 | LCDR3 |
|---|---|---|---|---|---|---|
| PI61 | SYGMH | VISYDGSN | SGYALHDD | TGTSSDV | DVSNRPS | SSYTSSS |
| | (SEQ ID | KYYADSV | YYGLDV | GGYNYV | (SEQ ID NO: | TLYV |
| | NO: 86) | KG (SEQ ID | (SEQ ID NO: | S (SEQ ID | 96) | (SEQ ID |
| | | NO: 87) | 88) | NO: 95) | | NO: 97) |
| B61-02 | SYGMH | VISYKGSN | SGYALHDD | TGTSSDV | EVSNRLR | SSYTSSS |
| | (SEQ ID | KYYADSV | YYGLDV | GGYNYV | (SEQ ID NO: | ALYV |
| | NO: 86) | KG (SEQ ID | (SEQ ID NO: | S (SEQ ID | 114) | (SEQ ID |
| | | NO: 109) | 88) | NO: 95) | | NO: 115) |
| B61-10 | SYGMH | VISYKGSN | SGYALHDD | TGTSSDV | EVSNRLR | SSYTSSS |
| | (SEQ ID | KYYADSV | YYGLDV | GGYNYV | (SEQ ID NO: | TLYV |
| | NO: 86) | KG (SEQ ID | (SEQ ID NO: | S (SEQ ID | 114) | (SEQ ID |
| | | NO: 109) | 88) | NO: 95) | | NO: 97) |
| Consensus | SYGMH | VISYXGSN | SGYALHDD | TGTSSDV | X₁VSNRX₂X₃, | SSYTSSS |
| | (SEQ ID | KYYADSV | YYGLDV | GGYNYV | wherein X₁ is | XLYV, |
| | NO: 86) | KG, wherein | (SEQ ID NO: | S (SEQ ID | D or E; X₂ is P | wherein X |
| | | X is D or K | 88) | NO: 95) | or L; and X₃ is | is T or A |
| | | (SEQ ID NO: | | | S or R (SEQ | (SEQ ID |
| | | 130) | | | ID NO: 131) | NO: 132) |

TABLE 9
Chothia CDRs of exemplary B cell-derived anti-BCMA molecules

| Chothia | HCDR1 | HCDR2 | HCDR3 | LCDR1 | LCDR2 | LCDR3 |
|---|---|---|---|---|---|---|
| PI61 | GFTFSSY | SYDGSN | SGYALHDDY | TSSDVGG | DVS (SEQ | YTSSSTLY |
| | (SEQ ID | (SEQ ID NO: | YGLDV (SEQ | YNY (SEQ | ID NO: 99) | (SEQ ID |
| | NO: 47) | 89) | ID NO: 88) | ID NO: 98) | | NO: 100) |
| B61-02 | GFTFSSY | SYKGSN | SGYALHDDY | TSSDVGG | EVS (SEQ | YTSSSAL |
| | (SEQ ID | (SEQ ID NO: | YGLDV (SEQ | YNY (SEQ | ID NO: | Y (SEQ ID |
| | NO: 47) | 110) | ID NO: 88) | ID NO: 98) | 116) | NO: 117) |
| B61-10 | GFTFSSY | SYKGSN | SGYALHDDY | TSSDVGG | EVS (SEQ | YTSSSTLY |
| | (SEQ ID | (SEQ ID NO: | YGLDV (SEQ | YNY (SEQ | ID NO: | (SEQ ID |
| | NO: 47) | 110) | ID NO: 88) | ID NO: 98) | 116) | NO: 100) |
| Consensus | GFTFSSY | SYXGSN, | SGYALHDDY | TSSDVGG | XVS, | YTSSSXL |
| | (SEQ ID | wherein X is | YGLDV (SEQ | YNY (SEQ | wherein X | Y, wherein |
| | NO: 47) | D or K (SEQ | ID NO: 88) | ID NO: 98) | is D or E | X is T or A |
| | | ID NO: 133) | | | (SEQ ID | (SEQ ID |
| | | | | | NO: 134) | NO: 135) |

TABLE 10
IMGT CDRs of exemplary B cell-derived anti-BCMA molecules

| IMGT | HCDR1 | HCDR2 | HCDR3 | LCDR1 | LCDR2 | LCDR3 |
|---|---|---|---|---|---|---|
| PI61 | GFTFSSYG | ISYDGSN | GGSGYALHDD | SSDVGGY | DVS | SSYTSSSTL |
| | (SEQ ID | K (SEQ ID | YYGLDV (SEQ | NY (SEQ | (SEQ ID | YV (SEQ ID |
| | NO: 90) | NO: 91) | ID NO: 92) | ID NO: 101) | NO: 99) | NO: 97) |
| B61-02 | GFTFSSYG | ISYKGSN | GGSGYALHDD | SSDVGGY | EVS | SSYTSSSA |
| | (SEQ ID | K (SEQ ID | YYGLDV (SEQ | NY (SEQ | (SEQ ID | LYV (SEQ |
| | NO: 90) | NO: 111) | ID NO: 92) | ID NO: 101) | NO: 116) | ID NO: 115) |
| B61-10 | GFTFSSYG | ISYKGSN | GGSGYALHDD | SSDVGGY | EVS | SSYTSSSTL |
| | (SEQ ID | K (SEQ ID | YYGLDV (SEQ | NY (SEQ | (SEQ ID | YV (SEQ ID |
| | NO: 90) | NO: 111) | ID NO: 92) | ID NO: 101) | NO: 116) | NO: 97) |
| Consensus | GFTFSSYG | ISYXGSN | GGSGYALHDD | SSDVGGY | XVS, | SSYTSSSX |
| | (SEQ ID | K, wherein | YYGLDV (SEQ | NY (SEQ | wherein | LYV, |
| | NO: 90) | X is D or K | ID NO: 92) | ID NO: 101) | X is D or | wherein X is |
| | | (SEQ ID | | | E (SEQ | T or A (SEQ |
| | | NO: 136) | | | ID NO: | ID NO: 132) |
| | | | | | 134) | |

TABLE 11
Amino acid and nucleic acid sequences of exemplary anti-BCMA molecules based on PI61

| Identification | Protein sequence | DNA sequence (5′-3′) |
|---|---|---|
| Signal peptide | MALPVTALLLPLALLLHAARP | Atggccctccctgtcaccgctctgttgctgccgcttgctctgctgctcc |
| | (SEQ ID NO: 1) | acgcagegegaccg (SEQ ID NO: 252) |
| PI61 VH | QVQLQESGGGVVQPGRSLRLS | CAGGTACAATTGCAGGAGTCTGGAGGCGG |
| | CAASGFTFSSYGMHWVRQAP | TGTGGTGCAACCCGGTCGCAGCTTGCGCCT |
| | GKGLEWVAVISYDGSNKYYA | GAGTTGTGCTGCGTCTGGATTTACATTTTC |
| | DSVKGRFTISRDNSKNTLYLQ | ATCTTACGGAATGCATTGGGTACGCCAGG |
| | MNSLRAEDTAVYYCGGSGYA | CACCGGGGAAAGGCCTTGAATGGGTGGCT |
| | LHDDYYGLDVWGQGTLVTVS | GTAATTTCATACGATGGTTCCAACAAATAC |
| | S (SEQ ID NO: 93) | TATGCTGACTCAGTCAAGGGTCGATTTACA |
| | | ATTAGTCGGGACAACTCCAAGAACACCCT |
| | | TTATCTTCAAATGAATTCCCTTAGAGCAGA |
| | | GGATACGGCGGTCTATTACTGTGGTGGCA |
| | | GTGGTTATGCACTTCATGATGATTACTATG |
| | | GCTTGGATGTCTGGGGGCAAGGGACGCTT |
| | | GTAACTGTATCCTCT (SEQ ID NO: 260) |
| PI61 VL | QSALTQPASVSGSPGQSITISCT | CAATCTGCTCTGACTCAACCAGCAAGCGT |
| | GTSSDVGGYNYVSWYQQHPG | ATCAGGGTCACCGGGACAGAGTATTACCA |
| | KAPKLMIYDVSNRPSGVSNRFS | TAAGTTGCACGGGGACCTCTAGCGATGTA |
| | GSKSGNTASLTISGLQAEDEAD | GGGGGGTATAATTATGTATCTTGGTATCAA |
| | YYCSSYTSSSTLYVFGSGTKVT | CAACACCCCGGGAAAGCCCCTAAATTGAT |
| | VL (SEQ ID NO: 102) | GATCTACGACGTGAGCAATCGACCTAGTG |
| | | GCGTATCAAATCGCTTCTCTGGTAGCAAGA |
| | | GTGGGAATACGGCGTCCCTTACTATTAGCG |
| | | GATTGCAAGCAGAAGATGAGGCCGATTAC |
| | | TACTGCAGCTCCTATACTAGCTCTTCTACA |
| | | TTGTACGTCTTTGGGAGCGGAACAAAAGT |
| | | AACAGTACTC (SEQ ID NO: 261) |
| Linker | GGGGSGGGGSGGGGS (SEQ ID | |
| | NO: 104) | |
| ScFv PI61 | QVQLQESGGGVVQPGRSLRLS | CaggtacaattgcaggagtctggaggcggtgtgGtgcaacccggtc |
| | CAASGFTFSSYGMHWVRQAP | gcagcttgcgcctgagttgtGctgcgtctggatttacattttcatcttac |
| | GKGLEWVAVISYDGSNKYYA | ggaAtgcattgggtacgccaggcaccggggaaaggcCttgaatgg |
| | DSVKGRFTISRDNSKNTLYLQ | gtggctgtaatttcatacgatggtTccaacaaatactatgctgactcag |
| | MNSLRAEDTAVYYCGGSGYA | tcaagggtCgatttacaattagtcgggacaactccaagaacAcccttt |
| | LHDDYYGLDVWGQGTLVTVS | atcttcaaatgaattcccttagagcaGaggatacggcggtctattactg |
| | SGGGGSGGGGSGGGGSQSALT | tggtggcagtGgttatgcacttcatgatgattactatggcttgGatgtct |
| | QPASVSGSPGQSITISCTGTSSD | gggggcaagggacgcttgtaactgtaTcctctggtggtggtggtagt |
| | VGGYNYVSWYQQHPGKAPKL | ggtgggggaggcTccggcggtggcggctctcaatctgctctgactC |
| | MIYDVSNRPSGVSNRFSGSKSG | aaccagcaagcgtatcagggtcaccgggacagAgtattaccataag |
| | NTASLTISGLQAEDEADYYCSS | ttgcacggggacctctagcGatgtaggggggtataattatgtatcttg |
| | YTSSSTLYVFGSGTKVTVL | gtatCaacaacaccccgggaaagcccctaaattgatgAtctacgac |
| | (SEQ ID NO: 105) | gtgagcaatcgacctagtggcgtaTcaaatcgcttctctggtagcaag |
| | | agtgggaatAcggcgtcccttactattagcggattgcaagcaGaag |
| | | atgaggccgattactactgcagctcctatActagctcttctacattgtac |
| | | gtctttgggagcggaacaaaagtaacagtactc (SEQ ID NO: |
| | | 253) |
| Transmembrane | TTTPAPRPPTPAPTIASQPLSLR | AcaacaacacctgccccgagaccgcctacaccaGccccgactatt |
| domain and hinge | PEACRPAAGGAVHTRGLDFAC | gccagccagcctctgagcctcAggcctgaggcctgtaggcccgca |
| | DIYIWAPLAGTCGVLLLSLVIT | gcgggcggcGcagttcatacacggggcttggatttcgcttgtGatatt |
| | LYC (SEQ ID NO: 202) | tatatttgggctcctttggcggggacaTgtggcgtgctgcttctgtcac |
| | | ttgttattacactgtactgt (SEQ ID NO: 254) |
| 4-1BB | KRGRKKLLYIFKQPFMRPVQT | AaacgcgggcgaaaaaaattgctgtatatttttAagcagccatttatg |
| | TQEEDGCSCRFPEEEEGGCEL | aggcccgttcagacgacgCaggaggaggacggttgctcttgcaggt |
| | (SEQ ID NO: 7) | tcccagaagaggaagaagggggctgtgaattg (SEQ ID NO: |
| | | 255) |
| CD3zeta | RVKFSRSADAPAYQQGQNQLY | CgggttaaattttcaagatccgcagacgctccaGcataccaacaggg |
| | NELNLGRREEYDVLDKRRGRD | acaaaaccaactctataacGagctgaatcttggaagaagggaggaat |
| | PEMGGKPRRKNPQEGLYNELQ | atgatGtgctggataaacggcgcggtagagatccggagAtgggcg |
| | KDKMAEAYSEIGMKGERRRG | gaaaaccaaggcgaaaaaaccctcagGagggactctacaacgaac |
| | KGHDGLYQGLSTATKDTYDA | tgcagaaagacaaaAtggcggaggcttattccgaaataggcatgaa |
| | LHMQALPPR (SEQ ID NO: 10) | gGgcgagcggaggcgagggaaagggcacgacggaCtgtatcaa |
| | | ggcctctcaaccgcgactaaggatAcgtacgacgccctgcacatgc |
| | | aggccctgcctccgaga (SEQ ID NO: 256) |
| PI61 full CAR | MALPVTALLLPLALLLHAARP | ATGGCCCTCCCTGTCACCGCTCTGTTGCTG |
| construct | QVQLQESGGGVVQPGRSLRLS | CCGCTTGCTCTGCTGCTCCACGCAGCGCGA |
| | CAASGFTFSSYGMHWVRQAP | CCGCAGGTACAATTGCAGGAGTCTGGAGG |
| | GKGLEWVAVISYDGSNKYYA | CGGTGTGGTGCAACCCGGTCGCAGCTTGC |
| | DSVKGRFTISRDNSKNTLYLQ | GCCTGAGTTGTGCTGCGTCTGGATTTACAT |
| | MNSLRAEDTAVYYCGGSGYA | TTTCATCTTACGGAATGCATTGGGTACGCC |
| | LHDDYYGLDVWGQGTLVTVS | AGGCACCGGGGAAAGGCCTTGAATGGGTG |
| | SGGGGSGGGGSGGGGSQSALT | GCTGTAATTTCATACGATGGTTCCAACAAA |
| | QPASVSGSPGQSITISCTGTSSD | TACTATGCTGACTCAGTCAAGGGTCGATTT |
| | VGGYNYVSWYQQHPGKAPKL | ACAATTAGTCGGGACAACTCCAAGAACAC |
| | MIYDVSNRPSGVSNRFSGSKSG | CCTTTATCTTCAAATGAATTCCCTTAGAGC |
| | NTASLTISGLQAEDEADYYCSS | AGAGGATACGGCGGTCTATTACTGTGGTG |
| | YTSSSTLYVFGSGTKVTVLTTT | GCAGTGGTTATGCACTTCATGATGATTACT |
| | PAPRPPTPAPTIASQPLSLRPEA | ATGGCTTGGATGTCTGGGGGCAAGGGACG |
| | CRPAAGGAVHTRGLDFACDIY | CTTGTAACTGTATCCTCTGGTGGTGGTGGT |
| | IWAPLAGTCGVLLLSLVITLYC | AGTGGTGGGGGAGGCTCCGGCGGTGGCGG |
| | KRGRKKLLYIFKQPFMRPVQT | CTCTCAATCTGCTCTGACTCAACCAGCAAG |
| | TQEEDGCSCRFPEEEEGGCELR | CGTATCAGGGTCACCGGGACAGAGTATTA |
| | VKFSRSADAPAYQQGQNQLY | CCATAAGTTGCACGGGGACCTCTAGCGAT |
| | NELNLGRREEYDVLDKRRGRD | GTAGGGGGGTATAATTATGTATCTTGGTAT |
| | PEMGGKPRRKNPQEGLYNELQ | CAACAACACCCCGGGAAAGCCCCTAAATT |
| | KDKMAEAYSEIGMKGERRRG | GATGATCTACGACGTGAGCAATCGACCTA |
| | KGHDGLYQGLSTATKDTYDA | GTGGCGTATCAAATCGCTTCTCTGGTAGCA |
| | LHMQALPPR (SEQ ID NO: 257) | AGAGTGGGAATACGGCGTCCCTTACTATT |
| | | AGCGGATTGCAAGCAGAAGATGAGGCCGA |
| | | TTACTACTGCAGCTCCTATACTAGCTCTTC |
| | | TACATTGTACGTCTTTGGGAGCGGAACAA |
| | | AAGTAACAGTACTCACAACAACACCTGCC |
| | | CCGAGACCGCCTACACCAGCCCCGACTAT |
| | | TGCCAGCCAGCCTCTGAGCCTCAGGCCTG |
| | | AGGCCTGTAGGCCCGCAGCGGGCGGCGCA |
| | | GTTCATACACGGGGCTTGGATTTCGCTTGT |
| | | GATATTTATATTTGGGCTCCTTTGGCGGGG |
| | | ACATGTGGCGTGCTGCTTCTGTCACTTGTT |
| | | ATTACACTGTACTGTAAACGCGGGCGAAA |
| | | AAAATTGCTGTATATTTTTAAGCAGCCATT |
| | | TATGAGGCCCGTTCAGACGACGCAGGAGG |
| | | AGGACGGTTGCTCTTGCAGGTTCCCAGAA |
| | | GAGGAAGAAGGGGGCTGTGAATTGCGGGT |
| | | TAAATTTTCAAGATCCGCAGACGCTCCAGC |
| | | ATACCAACAGGGACAAAACCAACTCTATA |
| | | ACGAGCTGAATCTTGGAAGAAGGGAGGAA |
| | | TATGATGTGCTGGATAAACGGCGCGGTAG |
| | | AGATCCGGAGATGGGCGGAAAACCAAGGC |
| | | GAAAAAACCCTCAGGAGGGACTCTACAAC |
| | | GAACTGCAGAAAGACAAAATGGCGGAGG |
| | | CTTATTCCGAAATAGGCATGAAGGGCGAG |
| | | CGGAGGCGAGGGAAAGGGCACGACGGAC |
| | | TGTATCAAGGCCTCTCAACCGCGACTAAG |
| | | GATACGTACGACGCCCTGCACATGCAGGC |
| | | CCTGCCTCCGAGA (SEQ ID NO: 258) |
| PI61 full CAR | | ATGGCCCTCCCTGTCACCGCTCTGTTGCTGCCGC |
| construct | | TTGCTCTGCTGCTCCACGCAGCGCGACCGCAGGT |
| (Nucleic acid | | ACAATTGCAGGAGTCTGGAGGCGGTGTGGTGCAA |
| with signal | | CCCGGTCGCAGCTTGCGCCTGAGTTGTGCTGCGT |
| peptide and stop | | CTGGATTTACATTTTCATCTTACGGAATGCATTG |
| codons) | | GGTACGCCAGGCACCGGGGAAAGGCCTTGAATGG |
| | | GTGGCTGTAATTTCATACGATGGTTCCAACAAAT |
| | | ACTATGCTGACTCAGTCAAGGGTCGATTTACAAT |
| | | TAGTCGGGACAACTCCAAGAACACCCTTTATCTT |
| | | CAAATGAATTCCCTTAGAGCAGAGGATACGGCGG |
| | | TCTATTACTGTGGTGGCAGTGGTTATGCACTTCA |
| | | TGATGATTACTATGGCTTGGATGTCTGGGGGCAA |
| | | GGGACGCTTGTAACTGTATCCTCTGGTGGTGGTG |
| | | GTAGTGGTGGGGGAGGCTCCGGCGGTGGCGGCTC |
| | | TCAATCTGCTCTGACTCAACCAGCAAGCGTATCA |
| | | GGGTCACCGGGACAGAGTATTACCATAAGTTGCA |
| | | CGGGGACCTCTAGCGATGTAGGGGGGTATAATTA |
| | | TGTATCTTGGTATCAACAACACCCCGGGAAAGCC |
| | | CCTAAATTGATGATCTACGACGTGAGCAATCGAC |
| | | CTAGTGGCGTATCAAATCGCTTCTCTGGTAGCAA |
| | | GAGTGGGAATACGGCGTCCCTTACTATTAGCGGA |
| | | TTGCAAGCAGAAGATGAGGCCGATTACTACTGCA |
| | | GCTCCTATACTAGCTCTTCTACATTGTACGTCTT |
| | | TGGGAGCGGAACAAAAGTAACAGTACTCACAACA |
| | | ACACCTGCCCCGAGACCGCCTACACCAGCCCCGA |
| | | CTATTGCCAGCCAGCCTCTGAGCCTCAGGCCTGA |
| | | GGCCTGTAGGCCCGCAGCGGGCGGCGCAGTTCAT |
| | | ACACGGGGCTTGGATTTCGCTTGTGATATTTATA |
| | | TTTGGGCTCCTTTGGCGGGGACATGTGGCGTGCT |
| | | GCTTCTGTCACTTGTTATTACACTGTACTGTAAA |
| | | CGCGGGCGAAAAAAATTGCTGTATATTTTTAAGC |
| | | AGCCATTTATGAGGCCCGTTCAGACGACGCAGGA |
| | | GGAGGACGGTTGCTCTTGCAGGTTCCCAGAAGAG |
| | | GAAGAAGGGGGCTGTGAATTGCGGGTTAAATTTT |
| | | CAAGATCCGCAGACGCTCCAGCATACCAACAGGG |
| | | ACAAAACCAACTCTATAACGAGCTGAATCTTGGA |
| | | AGAAGGGAGGAATATGATGTGCTGGATAAACGGC |
| | | GCGGTAGAGATCCGGAGATGGGCGGAAAACCAAG |
| | | GCGAAAAAACCCTCAGGAGGGACTCTACAACGAA |
| | | CTGCAGAAAGACAAAATGGCGGAGGCTTATTCCG |
| | | AAATAGGCATGAAGGGCGAGCGGAGGCGAGGGAA |
| | | AGGGCACGACGGACTGTATCAAGGCCTCTCAACC |
| | | GCGACTAAGGATACGTACGACGCCCTGCACATGC |
| | | AGGCCCTGCCTCCGAGATGATAA (SEQ ID |
| | | NO: 416) |
| PI61 mature | QVQLQESGGGVVQPGRSLRLS | caggtacaattgcaggagtctggaggcggtgtggtgcaacccggtc |
| CAR protein | CAASGFTFSSYGMHWVRQAP | gcagcttgcgcctgagttgtgctgcgtctggatttacattttcatcttacg |
| | GKGLEWVAVISYDGSNKYYA | gaatgcattgggtacgccaggcaccggggaaaggccttgaatgggt |
| | DSVKGRFTISRDNSKNTLYLQ | ggctgtaatttcatacgatggttccaacaaatactatgctgactcagtca |
| | MNSLRAEDTAVYYCGGSGYA | agggtcgatttacaattagtcgggacaactccaagaacaccctttatctt |
| | LHDDYYGLDVWGQGTLVTVS | caaatgaattcccttagagcagaggatacggcggtctattactgtggtg |
| | SGGGGSGGGGSGGGGSQSALT | gcagtggttatgcacttcatgatgattactatggcttggatgtctggggg |
| | QPASVSGSPGQSITISCTGTSSD | caagggacgcttgtaactgtatcctctggtggtggtggtagtggtggg |
| | VGGYNYVSWYQQHPGKAPKL | ggaggctccggcggtggcggctctcaatctgctctgactcaaccagc |
| | MIYDVSNRPSGVSNRFSGSKSG | aagcgtatcagggtcaccgggacagagtattaccataagttgcacgg |
| | NTASLTISGLQAEDEADYYCSS | ggacctctagcgatgtaggggggtataattatgtatcttggtatcaaca |
| | YTSSSTLYVFGSGTKVTVLTTT | acaccccgggaaagcccctaaattgatgatctacgacgtgagcaatc |
| | PAPRPPTPAPTIASQPLSLRPEA | gacctagtggcgtatcaaatcgcttctctggtagcaagagtgggaata |
| | CRPAAGGAVHTRGLDFACDIY | cggcgtcccttactattagcggattgcaagcagaagatgaggccgatt |
| | IWAPLAGTCGVLLLSLVITLYC | actactgcagctcctatactagctcttctacattgtacgtctttgggagcg |
| | KRGRKKLLYIFKQPFMRPVQT | gaacaaaagtaacagtactcacaacaacacctgccccgagaccgect |
| | TQEEDGCSCRFPEEEEGGCELR | acaccagccccgactattgccagccagcctctgagcctcaggcctga |
| | VKFSRSADAPAYQQGQNQLY | ggcctgtaggcccgcagcgggcggcgcagttcatacacggggcttg |
| | NELNLGRREEYDVLDKRRGRD | gatttcgcttgtgatatttatatttgggctcctttggcggggacatgtggc |
| | PEMGGKPRRKNPQEGLYNELQ | gtgctgcttctgtcacttgttattacactgtactgtaaacgcgggcgaaa |
| | KDKMAEAYSEIGMKGERRRG | aaaattgctgtatatttttaagcagccatttatgaggcccgttcagacga |
| | KGHDGLYQGLSTATKDTYDA | cgcaggaggaggacggttgctcttgcaggttcccagaagaggaaga |
| | LHMQALPPR (SEQ ID NO: 107) | agggggctgtgaattgcgggttaaattttcaagatccgcagacgctcc |
| | | agcataccaacagggacaaaaccaactctataacgagctgaatcttg |
| | | gaagaagggaggaatatgatgtgctggataaacggcgcggtagaga |
| | | tccggagatgggcggaaaaccaaggcgaaaaaaccctcaggaggg |
| | | actctacaacgaactgcagaaagacaaaatggcggaggcttattccg |
| | | aaataggcatgaagggcgagcggaggcgagggaaagggcacgac |
| | | ggactgtatcaaggcctctcaaccgcgactaaggatacgtacgacgc |
| | | cctgcacatgcaggccctgcctccgaga (SEQ ID NO: 259) |

TABLE 11A
Additional exemplary anti-BCMA binder sequences based on PI161

| SEQ ID | | |
|---|---|---|
| NO | Region | Sequence |
| 400 | VH | CAGGTGCAGCTGCAGGAGTCCGGCGGCGGCGTGGTGCAGCCAGGCC |
| | | GGTCCCTGAGACTGTCTTGTGCCGCCAGCGGCTTCACCTTTTCCTCTT |
| | | ATGGCATGCACTGGGTGAGACAGGCACCTGGCAAGGGACTGGAGTG |
| | | GGTGGCCGTGATCTCCTACGACGGCTCTAACAAGTATTACGCCGATA |
| | | GCGTGAAGGGCAGGTTCACCATCAGCCGCGACAACTCCAAGAATACA |
| | | CTGTATCTGCAGATGAATAGCCTGCGGGCCGAGGATACCGCCGTGTA |
| | | TTACTGCGGAGGCTCCGGCTACGCACTGCACGACGATTATTACGGAC |
| | | TGGACGTGTGGGGACAGGGCACCCTGGTCACAGTGAGCTCC |
| 401 | VH | CAGGTGCAGCTGCAGGAGTCTGGCGGAGGAGTGGTGCAGCCAGGCC |
| | | GGTCCCTGAGACTGTCTTGCGCCGCCAGCGGCTTCACATTTTCTAGCT |
| | | ACGGAATGCACTGGGTGCGCCAGGCACCTGGCAAGGGACTGGAGTG |
| | | GGTGGCCGTGATCTCCTATGACGGCTCTAACAAGTACTATGCCGATTC |
| | | CGTGAAGGGCAGGTTCACCATCAGCCGCGACAACTCCAAGAATACAC |
| | | TGTACCTGCAGATGAATTCCCTGCGGGCCGAGGATACCGCCGTGTAC |
| | | TATTGTGGCGGCTCTGGCTATGCCCTGCACGACGATTACTATGGACTG |
| | | GACGTGTGGGGACAGGGCACCCTGGTGACAGTGTCCTCT |
| 402 | VH | CAGGTGCAGCTGCAGGAGTCTGGCGGAGGAGTGGTGCAGCCAGGCC |
| | | GGAGCCTGAGACTGTCCTGCGCCGCCTCTGGCTTCACCTTTAGCTCCT |
| | | ATGGCATGCACTGGGTGAGACAGGCACCTGGCAAGGGACTGGAGTG |
| | | GGTGGCCGTGATCAGCTACGACGGCTCCAACAAGTATTACGCCGATA |
| | | GCGTGAAGGGCAGGTTCACCATCTCTCGCGACAACAGCAAGAATACA |
| | | CTGTATCTGCAGATGAATTCCCTGCGGGCCGAGGATACAGCCGTGTA |
| | | TTACTGCGGAGGCAGCGGCTACGCACTGCACGACGATTATTACGGAC |
| | | TGGACGTGTGGGGACAGGGCACCCTGGTCACAGTGTCTAGC |
| 403 | VH | CAGGTGCAGCTGCAGGAGAGCGGCGGCGGCGTGGTGCAGCCCGGCC |
| | | GGTCTCTGAGACTGAGCTGTGCCGCCTCCGGCTTCACCTTTAGCTCCT |
| | | ACGGAATGCACTGGGTGCGCCAGGCACCTGGCAAGGGACTGGAGTG |
| | | GGTGGCCGTGATCTCTTATGACGGCAGCAACAAGTACTATGCCGATA |
| | | GCGTGAAGGGCAGGTTCACCATCTCCCGCGACAACTCTAAGAATACA |
| | | CTGTACCTGCAGATGAATAGCCTGCGGGCCGAGGATACCGCCGTGTA |
| | | CTATTGCGGAGGCTCCGGCTATGCACTGCACGACGATTACTATGGAC |
| | | TGGACGTGTGGGGACAGGGCACCCTGGTGACAGTGTCTAGC |
| 404 | VH | CAGGTCCAGCTGCAGGAGAGTGGGGGGGGGGTCGTCCAGCCCGGAA |
| | | GAAGCCTGAGACTGTCATGTGCCGCATCTGGGTTTACTTTTAGCTCCT |
| | | ATGGAATGCACTGGGTGCGCCAGGCACCTGGCAAGTGCCTGGAGTGG |
| | | GTGGCCGTGATCTCCTACGACGGCTCTAACAAGTACTATGCCGATAG |
| | | CGTGAAGGGCCGGTTCACCATCAGCAGAGACAACTCCAAGAATACAC |
| | | TGTATCTGCAGATGAATTCTCTGCGGGCCGAGGATACCGCCGTGTACT |
| | | ATTGTGGAGGCTCCGGCTACGCACTGCACGACGATTACTATGGACTG |
| | | GACGTGTGGGGACAGGGCACCCTGGTGACAGTGTCTAGC |
| 405 | VH | CAGGTCCAGCTGCAGGAATCCGGCGGAGGAGTGGTGCAGCCAGGCC |
| | | GGTCTCTGAGACTGAGCTGCGCCGCCTCCGGCTTCACATTTTCCTCTT |
| | | ATGGCATGCACTGGGTGAGACAGGCCCCTGGCAAGTGTCTGGAGTGG |
| | | GTGGCCGTGATCTCTTACGACGGCAGCAACAAGTATTACGCCGATAG |
| | | CGTGAAGGGCAGGTTCACCATCTCCCGCGACAACTCTAAGAATACAC |
| | | TGTATCTGCAGATGAATTCCCTGCGGGCCGAGGATACCGCCGTGTATT |
| | | ACTGCGGCGGCTCTGGCTACGCCCTGCACGACGACTACTATGGACTG |
| | | GATGTCTGGGGGCAGGGCACACTGGTCACTGTCTCTTCA |
| 406 | VH | CAGGTCCAGCTGCAGGAATCAGGGGGGGGGGTCGTCCAGCCCGGAA |
| | | GAAGTCTGAGACTGTCATGTGCCGCATCAGGGTTTACCTTTAGCTCCT |
| | | ATGGAATGCACTGGGTGCGCCAGGCACCTGGCAAGTGCCTGGAGTGG |
| | | GTGGCCGTGATCTCCTACGACGGCTCTAACAAGTACTATGCCGATAG |
| | | CGTGAAGGGCCGGTTCACCATCAGCAGAGACAACTCCAAGAATACAC |
| | | TGTATCTGCAGATGAATTCTCTGCGGGCCGAGGATACCGCCGTGTACT |
| | | ATTGTGGAGGCTCCGGCTACGCACTGCACGACGATTACTATGGACTG |
| | | GACGTGTGGGGACAGGGCACCCTGGTGACAGTGTCTAGC |
| 407 | VH | CAGGTCCAGCTGCAGGAATCCGGCGGAGGAGTGGTGCAGCCAGGCC |
| | | GGTCTCTGAGACTGAGCTGCGCCGCCTCCGGCTTCACCTTTTCCTCTT |
| | | ATGGCATGCACTGGGTGAGACAGGCCCCTGGCAAGTGTCTGGAGTGG |
| | | GTGGCCGTGATCTCTTACGACGGCAGCAACAAGTATTACGCCGATAG |
| | | CGTGAAGGGCAGGTTCACCATCTCCCGCGACAACTCTAAGAATACAC |
| | | TGTATCTGCAGATGAATTCCCTGCGGGCCGAGGATACAGCCGTGTAT |
| | | TACTGTGGCGGCTCTGGCTACGCCCTGCATGATGATTATTATGGACTG |
| | | GATGTCTGGGGGCAGGGCACACTGGTCACTGTCTCTTCC |
| 408 | VL | CAGTCTGCCCTGACCCAGCCAGCAAGCGTGTCCGGCTCTCCTGGCCA |
| | | GAGCATCACAATCTCCTGCACCGGCACAAGCTCCGACGTGGGAGGCT |
| | | ATAACTACGTGAGCTGGTATCAGCAGCACCCAGGCAAGGCCCCCAAG |
| | | CTGATGATCTACGACGTGAGCAACAGGCCTTCTGGCGTGAGCAATCG |
| | | CTTCAGCGGCTCCAAGTCTGGCAATACCGCCTCTCTGACAATCAGCG |
| | | GCCTGCAGGCAGAGGACGAGGCAGATTATTACTGCTCTAGCTATACC |
| | | TCCTCTAGCACACTGTACGTGTTTGGCAGCGGCACCAAGGTGACAGT |
| | | GCTG |
| 409 | VL | CAGAGCGCCCTGACCCAGCCAGCATCCGTGTCTGGCAGCCCAGGCCA |
| | | GTCTATCACAATCAGCTGCACCGGCACAAGCTCCGACGTGGGAGGCT |
| | | ACAACTATGTGAGCTGGTACCAGCAGCACCCTGGCAAGGCCCCAAAG |
| | | CTGATGATCTATGACGTGAGCAACCGGCCATCCGGCGTGTCTAATAG |
| | | ATTCTCCGGCTCTAAGAGCGGCAATACCGCCTCCCTGACAATCTCTGG |
| | | CCTGCAGGCAGAGGACGAGGCAGATTACTATTGTTCTAGCTACACCT |
| | | CCTCTAGCACACTGTACGTGTTCGGCAGCGGCACCAAGGTGACAGTG |
| | | CTG |
| 410 | VL | CAGTCTGCCCTGACCCAGCCAGCAAGCGTGTCCGGCTCTCCTGGCCA |
| | | GTCCATCACAATCTCTTGTACCGGCACATCCTCTGACGTGGGCGGCTA |
| | | TAACTACGTGTCCTGGTATCAGCAGCACCCAGGCAAGGCCCCCAAGC |
| | | TGATGATCTACGATGTGAGCAACAGGCCTTCTGGCGTGAGCAATCGC |
| | | TTCAGCGGCTCCAAGTCTGGCAATACCGCCAGCCTGACAATCTCCGG |
| | | CCTGCAGGCAGAGGACGAGGCAGATTATTACTGCAGCTCCTATACCT |
| | | CTAGCTCCACACTGTACGTGTTTGGCAGCGGCACCAAGGTGACAGTG |
| | | CTG |
| 411 | VL | CAGAGCGCCCTGACCCAGCCAGCATCCGTGTCTGGCAGCCCAGGCCA |
| | | GTCCATCACAATCTCTTGCACCGGCACATCTAGCGACGTGGGCGGCT |
| | | ACAACTACGTGAGCTGGTACCAGCAGCACCCTGGCAAGGCCCCAAAG |
| | | CTGATGATCTATGATGTGAGCAACCGGCCCTCCGGCGTGTCTAATAG |
| | | ATTCTCCGGCTCTAAGAGCGGCAATACCGCCAGCCTGACAATCTCCG |
| | | GCCTGCAGGCAGAGGACGAGGCAGATTACTATTGCTCCTCTTACACC |
| | | AGCTCCTCTACACTGTACGTGTTCGGCTCCGGCACCAAGGTGACAGT |
| | | GCTG |
| 412 | VL | CAGTCTGCCCTGACCCAGCCTGCAAGCGTGTCCGGCTCTCCAGGCCA |
| | | GTCTATCACAATCAGCTGTACCGGCACAAGCTCCGACGTGGGCGGCT |
| | | ATAACTACGTGAGCTGGTATCAGCAGCACCCTGGCAAGGCCCCAAAG |
| | | CTGATGATCTACGACGTGAGCAACCGGCCCTCTGGCGTGAGCAATCG |
| | | GTTCAGCGGCAGCAAGTCTGGCAATACCGCCTCCCTGACAATCTCTG |
| | | GCCTGCAGGCAGAGGACGAGGCAGATTATTACTGTAGCAGTTATACT |
| | | TCAAGCTCAACCCTGTACGTGTTTGGATGCGGCACTAAGGTCACCGT |
| | | CCTG |
| 413 | VL | CAGTCTGCTCTGACCCAGCCCGCTTCCGTCTCAGGGTCTCCAGGACAG |
| | | TCAATTACCATTAGTTGCACAGGCACCTCATCCGATGTGGGCGGCTAT |
| | | AACTACGTGTCCTGGTATCAGCAGCACCCAGGCAAGGCCCCCAAGCT |
| | | GATGATCTACGACGTGAGCAACAGGCCATCTGGCGTGAGCAATCGCT |
| | | TCAGCGGCTCCAAGTCTGGCAATACCGCCAGCCTGACAATCTCCGGC |
| | | CTGCAGGCAGAGGACGAGGCAGATTACTATTGCAGCTCCTATACCTC |
| | | TAGCTCCACACTGTACGTGTTTGGCTGTGGCACCAAGGTGACAGTGCT |
| | | G |
| 414 | VL | CAGTCTGCCCTGACCCAGCCTGCAAGCGTGTCCGGCTCTCCAGGCCA |
| | | GTCTATCACAATCAGCTGTACCGGCACAAGCTCCGACGTGGGCGGCT |
| | | ATAACTACGTGAGCTGGTATCAGCAGCACCCTGGCAAGGCCCCAAAG |
| | | CTGATGATCTACGACGTGAGCAACCGGCCCTCTGGCGTGAGCAATCG |
| | | GTTCAGCGGCAGCAAGTCTGGCAATACCGCCTCCCTGACAATCTCTG |
| | | GCCTGCAGGCAGAGGACGAGGCAGATTATTACTGTAGCTCCTACACT |
| | | TCTTCAAGCACACTGTATGTCTTTGGATGCGGAACTAAGGTCACTGTC |
| | | CTG |
| 415 | VL | CAGTCTGCTCTGACCCAGCCCGCTTCCGTCTCAGGATCTCCAGGACAG |
| | | TCTATTACAATTAGTTGCACAGGAACCTCTTCCGATGTGGGCGGCTAT |
| | | AACTACGTGTCCTGGTATCAGCAGCACCCAGGCAAGGCCCCCAAGCT |
| | | GATGATCTACGACGTGAGCAACAGGCCTTCTGGCGTGAGCAATCGCT |
| | | TCAGCGGCTCCAAGTCTGGCAATACCGCCAGCCTGACAATCTCCGGC |
| | | CTGCAGGCAGAGGACGAGGCAGATTACTATTGCAGCTCCTATACCTC |
| | | TAGCTCCACACTGTACGTGTTTGGCTGTGGCACCAAGGTGACAGTGCT |
| | | G |
| SEQ ID | Ant- | QVQLQESGGGVVQPGRSLRLSCAASGFTFSSYGMHWVRQAPGKGLEW |
| NO: 93 | BCMA VH | VAVISYDGSNKYYADSVKGRFTISRDNSKNTLYLQMNSLRAEDTAVYY |
| | (PI61) | CGGSGYALHDDYYGLDVWGQGTLVTVSS |
| SEQ ID | Anti- | QSALTQPASVSGSPGQSITISCTGTSSDVGGYNYVSWYQQHPGKAPKLMI |
| NO: 102 | BCMA VL | YDVSNRPSGVSNRFSGSKSGNTASLTISGLQAEDEADYYCSSYTSSSTLY |
| | (PI61) | VFGSGTKVTVL |
| SEQ ID | Anti- | QVQLQESGGGVVQPGRSLRLSCAASGFTFSSYGMHWVRQAPGKCLEW |
| NO: 333 | BCMA VH | VAVISYDGSNKYYADSVKGRFTISRDNSKNTLYLQMNSLRAEDTAVYY |
| | (PI61) | CGGSGYALHDDYYGLDVWGQGTLVTVSS |
| | variant | |
| SEQ ID | Anti- | QSALTQPASVSGSPGQSITISCTGTSSDVGGYNYVSWYQQHPGKAPKLMI |
| NO: 334 | BCMA VL | YDVSNRPSGVSNRFSGSKSGNTASLTISGLQAEDEADYYCSSYTSSSTLY |
| | (PI61) | VFGCGTKVTVL |
| | variant | |

TABLE 12
Amino acid and nucleic acid sequences
of exemplary hybridoma-derived anti-BCMA molecules

| SEQ ID | Name/ | |
|---|---|---|
| NO | Description | Sequence |
| Hy03 | | |
| SEQ ID | HCDR1 | GFWMS |
| NO: 137 | (Kabat) | |
| SEQ ID | HCDR2 | NIKQDGSEKYYVDSVRG |
| NO: 138 | (Kabat) | |
| SEQ ID | HCDR3 | ALDYYGMDV |
| NO: 139 | (Kabat) | |
| SEQ ID | HCDR1 | GFTFSGF |
| NO: 140 | (Chothia) | |
| SEQ ID | HCDR2 | KQDGSE |
| NO: 141 | (Chothia) | |
| SEQ ID | HCDR3 | ALDYYGMDV |
| NO: 139 | (Chothia) | |
| SEQ ID | HCDR1 | GFTFSGFW |
| NO: 142 | (IMGT) | |
| SEQ ID | HCDR2 | IKQDGSEK |
| NO: 143 | (IMGT) | |
| SEQ ID | HCDR3 | ARALDYYGMDV |
| NO: 144 | (IMGT) | |
| SEQ ID | VH | EVQLVESGGGLVQPGGSLRLSCAASGFTFSGFWMSWVRQAPGKGLEWV |
| NO: 145 | | ANIKQDGSEKYYVDSVRGRFTISRDNAKNSLYLQMNSLRAEDTAVYYCA |
| | | RALDYYGMDVWGQGTTVTVSS |
| SEQ ID | DNA VH | GAAGTGCAACTGGTGGAGAGCGGTGGAGGGCTTGTCCAGCCCGGAGG |
| NO: 146 | | ATCGCTGCGGCTGTCCTGTGCTGCGTCCGGGTTCACCTTCTCCGGCTTC |
| | | TGGATGTCCTGGGTCAGACAGGCACCGGGAAAGGGCCTCGAATGGGT |
| | | GGCCAACATCAAGCAGGATGGCTCCGAGAAGTACTACGTCGACTCCGT |
| | | GAGAGGCCGCTTCACCATCTCCCGGGACAACGCCAAGAACTCGCTGTA |
| | | CCTCCAAATGAATAGCCTCAGGGCGGAAGATACTGCTGTGTATTACTG |
| | | CGCACGCGCCCTTGACTACTACGGCATGGACGTCTGGGGCCAAGGGAC |
| | | CACTGTGACCGTGTCTAGC |
| SEQ ID | LCDR1 | RSSQSLLDSDDGNTYLD |
| NO: 147 | (Kabat) | |
| SEQ ID | LCDR2 | TLSYRAS |
| NO: 148 | (Kabat) | |
| SEQ ID | LCDR3 | TQRLEFPSIT |
| NO: 149 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RLEFPSI |
| NO: 152 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | TQRLEFPSIT |
| NO: 149 | (IMGT) | |
| SEQ ID | VL | DIVMTQTPLSLPVTPGEPASISCRSSQSLLDSDDGNTYLDWYLQKPGQSPR |
| NO: 154 | | LLIYTLSYRASGVPDRFSGSGSGTDFTLKISRVEAEDVGLYYCTQRLEFPSI |
| | | TFGQGTRLEIK |
| SEQ ID | DNA VL | GATATCGTGATGACCCAGACTCCCCTGTCCCTGCCTGTGACTCCCGGA |
| NO: 155 | | GAACCAGCCTCCATTTCCTGCCGGTCCTCCCAGTCCCTGCTGGACAGC |
| | | GACGACGGCAACACTTACCTGGACTGGTACTTGCAGAAGCCGGGCCA |
| | | ATCGCCTCGCCTGCTGATCTATACCCTGTCATACCGGGCCTCAGGAGT |
| | | GCCTGACCGCTTCTCGGGATCAGGGAGCGGGACCGATTTCACCCTGAA |
| | | AATTTCCCGAGTGGAAGCCGAGGACGTCGGACTGTACTACTGCACCCA |
| | | GCGCCTCGAATTCCCGTCGATTACGTTTGGACAGGGTACCCGGCTTGA |
| | | GATCAAG |
| SEQ ID | Linker | GGGGSGGGGSGGGGSGGGGS |
| NO: 63 | | |
| SEQ ID | scFv (VH- | EVQLVESGGGLVQPGGSLRLSCAASGFTFSGFWMSWVRQAPGKGLEWV |
| NO: 156 | linker-VL) | ANIKQDGSEKYYVDSVRGRFTISRDNAKNSLYLQMNSLRAEDTAVYYCA |
| | | RALDYYGMDVWGQGTTVTVSSGGGGSGGGGSGGGGSGGGGSDIVMTQ |
| | | TPLSLPVTPGEPASISCRSSQSLLDSDDGNTYLDWYLQKPGQSPRLLIYTLS |
| | | YRASGVPDRFSGSGSGTDFTLKISRVEAEDVGLYYCTQRLEFPSITFGQGT |
| | | RLEIK |
| SEQ ID | DNA scFv | GAAGTGCAACTGGTGGAGAGCGGTGGAGGGCTTGTCCAGCCCGGAGG |
| NO: 157 | | ATCGCTGCGGCTGTCCTGTGCTGCGTCCGGGTTCACCTTCTCCGGCTTC |
| | | TGGATGTCCTGGGTCAGACAGGCACCGGGAAAGGGCCTCGAATGGGT |
| | | GGCCAACATCAAGCAGGATGGCTCCGAGAAGTACTACGTCGACTCCGT |
| | | GAGAGGCCGCTTCACCATCTCCCGGGACAACGCCAAGAACTCGCTGTA |
| | | CCTCCAAATGAATAGCCTCAGGGCGGAAGATACTGCTGTGTATTACTG |
| | | CGCACGCGCCCTTGACTACTACGGCATGGACGTCTGGGGCCAAGGGAC |
| | | CACTGTGACCGTGTCTAGCGGAGGCGGAGGTTCAGGGGGCGGTGGAT |
| | | CAGGCGGAGGAGGATCGGGGGGTGGTGGATCGGATATCGTGATGACC |
| | | CAGACTCCCCTGTCCCTGCCTGTGACTCCCGGAGAACCAGCCTCCATTT |
| | | CCTGCCGGTCCTCCCAGTCCCTGCTGGACAGCGACGACGGCAACACTT |
| | | ACCTGGACTGGTACTTGCAGAAGCCGGGCCAATCGCCTCGCCTGCTGA |
| | | TCTATACCCTGTCATACCGGGCCTCAGGAGTGCCTGACCGCTTCTCGG |
| | | GATCAGGGAGCGGGACCGATTTCACCCTGAAAATTTCCCGAGTGGAA |
| | | GCCGAGGACGTCGGACTGTACTACTGCACCCAGCGCCTCGAATTCCCG |
| | | TCGATTACGTTTGGACAGGGTACCCGGCTTGAGATCAAG |
| SEQ ID | Full CAR | EVQLVESGGGLVQPGGSLRLSCAASGFTFSGFWMSWVRQAPGKGLEWV |
| NO: 158 | amino acid | ANIKQDGSEKYYVDSVRGRFTISRDNAKNSLYLQMNSLRAEDTAVYYCA |
| | sequence | RALDYYGMDVWGQGTTVTVSSGGGGSGGGGSGGGGSGGGGSDIVMTQ |
| | | TPLSLPVTPGEPASISCRSSQSLLDSDDGNTYLDWYLQKPGQSPRLLIYTLS |
| | | YRASGVPDRFSGSGSGTDFTLKISRVEAEDVGLYYCTQRLEFPSITFGQGT |
| | | RLEIKTTTPAPRPPTPAPTIASQPLSLRPEACRPAAGGAVHTRGLDFACDIYI |
| | | WAPLAGTCGVLLLSLVITLYCKRGRKKLLYIFKQPFMRPVQTTQEEDGCS |
| | | CRFPEEEEGGCELRVKFSRSADAPAYQQGQNQLYNELNLGRREEYDVLD |
| | | KRRGRDPEMGGKPRRKNPQEGLYNELQKDKMAEAYSEIGMKGERRRGK |
| | | GHDGLYQGLSTATKDTYDALHMQALPPR |
| SEQ ID | Full CAR | GAAGTGCAACTGGTGGAGAGCGGTGGAGGGCTTGTCCAGCCCGGAGG |
| NO: 159 | DNA | ATCGCTGCGGCTGTCCTGTGCTGCGTCCGGGTTCACCTTCTCCGGCTTC |
| | sequence | TGGATGTCCTGGGTCAGACAGGCACCGGGAAAGGGCCTCGAATGGGT |
| | | GGCCAACATCAAGCAGGATGGCTCCGAGAAGTACTACGTCGACTCCGT |
| | | GAGAGGCCGCTTCACCATCTCCCGGGACAACGCCAAGAACTCGCTGTA |
| | | CCTCCAAATGAATAGCCTCAGGGCGGAAGATACTGCTGTGTATTACTG |
| | | CGCACGCGCCCTTGACTACTACGGCATGGACGTCTGGGGCCAAGGGAC |
| | | CACTGTGACCGTGTCTAGCGGAGGCGGAGGTTCAGGGGGCGGTGGAT |
| | | CAGGCGGAGGAGGATCGGGGGGTGGTGGATCGGATATCGTGATGACC |
| | | CAGACTCCCCTGTCCCTGCCTGTGACTCCCGGAGAACCAGCCTCCATTT |
| | | CCTGCCGGTCCTCCCAGTCCCTGCTGGACAGCGACGACGGCAACACTT |
| | | ACCTGGACTGGTACTTGCAGAAGCCGGGCCAATCGCCTCGCCTGCTGA |
| | | TCTATACCCTGTCATACCGGGCCTCAGGAGTGCCTGACCGCTTCTCGG |
| | | GATCAGGGAGCGGGACCGATTTCACCCTGAAAATTTCCCGAGTGGAA |
| | | GCCGAGGACGTCGGACTGTACTACTGCACCCAGCGCCTCGAATTCCCG |
| | | TCGATTACGTTTGGACAGGGTACCCGGCTTGAGATCAAGACCACTACC |
| | | CCAGCACCGAGGCCACCCACCCCGGCTCCTACCATCGCCTCCCAGCCT |
| | | CTGTCCCTGCGTCCGGAGGCATGTAGACCCGCAGCTGGTGGGGCCGTG |
| | | CATACCCGGGGTCTTGACTTCGCCTGCGATATCTACATTTGGGCCCCTC |
| | | TGGCTGGTACTTGCGGGGTCCTGCTGCTTTCACTCGTGATCACTCTTTA |
| | | CTGTAAGCGCGGTCGGAAGAAGCTGCTGTACATCTTTAAGCAACCCTT |
| | | CATGAGGCCTGTGCAGACTACTCAAGAGGAGGACGGCTGTTCATGCCG |
| | | GTTCCCAGAGGAGGAGGAAGGCGGCTGCGAACTGCGCGTGAAATTCA |
| | | GCCGCAGCGCAGATGCTCCAGCCTACCAGCAGGGGCAGAACCAGCTC |
| | | TACAACGAACTCAATCTTGGTCGGAGAGAGGAGTACGACGTGCTGGA |
| | | CAAGCGGAGAGGACGGGACCCAGAAATGGGCGGGAAGCCGCGCAGA |
| | | AAGAATCCCCAAGAGGGCCTGTACAACGAGCTCCAAAAGGATAAGAT |
| | | GGCAGAAGCCTATAGCGAGATTGGTATGAAAGGGGAACGCAGAAGAG |
| | | GCAAAGGCCACGACGGACTGTACCAGGGACTCAGCACCGCCACCAAG |
| | | GACACCTATGACGCTCTTCACATGCAGGCCCTGCCGCCTCGG |
| Hy52 | | |
| SEQ ID | HCDR1 | SFRMN |
| NO: 160 | (Kabat) | |
| SEQ ID | HCDR2 | SISSSSSYIYYADSVKG |
| NO: 161 | (Kabat) | |
| SEQ ID | HCDR3 | WLSYYGMDV |
| NO: 162 | (Kabat) | |
| SEQ ID | HCDR1 | GFTFSSF |
| NO: 163 | (Chothia) | |
| SEQ ID | HCDR2 | SSSSSY |
| NO: 164 | (Chothia) | |
| SEQ ID | HCDR3 | WLSYYGMDV |
| NO: 162 | (Chothia) | |
| SEQ ID | HCDR1 | GFTFSSFR |
| NO: 165 | (IMGT) | |
| SEQ ID | HCDR2 | ISSSSSYI |
| NO: 166 | (IMGT) | |
| SEQ ID | HCDR3 | ARWLSYYGMDV |
| NO: 167 | (IMGT) | |
| SEQ ID | VH | EVQLVESGGGLVKPGGSLRLSCAASGFTFSSFRMNWVRQAPGKGLEWVS |
| NO: 168 | | SISSSSSYIYYADSVKGRFTISRDNAKNSLYLQMNSLRAEDTAVYYCARW |
| | | LSYYGMDVWGQGTTVTVSS |
| SEQ ID | DNA VH | GAAGTGCAACTGGTGGAGAGCGGTGGAGGGCTTGTCAAGCCCGGAGG |
| NO: 169 | | ATCGCTGCGGCTGTCCTGTGCTGCGTCCGGGTTCACCTTCTCCTCGTTC |
| | | CGCATGAACTGGGTCAGACAGGCACCGGGAAAGGGCCTCGAATGGGT |
| | | GTCCTCAATCTCATCGTCCTCGTCCTACATCTACTACGCCGACTCCGTG |
| | | AAAGGCCGCTTCACCATCTCCCGGGACAACGCCAAGAACTCGCTGTAC |
| | | CTCCAAATGAATAGCCTCAGGGCGGAAGATACTGCTGTGTATTACTGC |
| | | GCACGCTGGCTTTCCTACTACGGCATGGACGTCTGGGGCCAAGGGACC |
| | | ACTGTGACCGTGTCTAGC |
| SEQ ID | LCDR1 | RSSQSLLDSDDGNTYLD |
| NO: 147 | (Kabat) | |
| SEQ ID | LCDR2 | TLSFRAS |
| NO: 170 | (Kabat) | |
| SEQ ID | LCDR3 | MQRIGFPIT |
| NO: 171 | (Kabat) | |
| SEQ ID | LCDR1 | SQSLLDSDDGNTY |
| NO: 150 | (Chothia) | |
| SEQ ID | LCDR2 | TLS |
| NO: 151 | (Chothia) | |
| SEQ ID | LCDR3 | RIGFPI |
| NO: 172 | (Chothia) | |
| SEQ ID | LCDR1 | QSLLDSDDGNTY |
| NO: 153 | (IMGT) | |
| SEQ ID | LCDR2 | TLS |
| NO: 151 | (IMGT) | |
| SEQ ID | LCDR3 | MQRIGFPIT |
| NO: 171 | (IMGT) | |
| SEQ ID | VL | DIVMTQTPLSLPVTPGEPASISCRSSQSLLDSDDGNTYLDWYLQKPGQSPQ |
| NO: 173 | | LLIYTLSFRASGVPDRFSGSGSGTDFTLKIRRVEAEDVGVYYCMQRIGFPIT |
| | | FGQGTRLEIK |
| SEQ ID | DNA VL | GATATCGTGATGACCCAGACTCCCCTGTCCCTGCCTGTGACTCCCGGA |
| NO: 174 | | GAACCAGCCTCCATTTCCTGCCGGTCCTCCCAGTCCCTGCTGGACAGC |
| | | GACGACGGCAACACTTACCTGGACTGGTACTTGCAGAAGCCGGGCCA |
| | | ATCGCCTCAGCTGCTGATCTATACCCTGTCATTCCGGGCCTCAGGAGT |
| | | GCCTGACCGCTTCTCGGGATCAGGGAGCGGGACCGATTTCACCCTGAA |
| | | AATTAGGCGAGTGGAAGCCGAGGACGTCGGAGTGTACTACTGCATGC |
| | | AGCGCATCGGCTTCCCGATTACGTTTGGACAGGGTACCCGGCTTGAGA |
| | | TCAAG |
| SEQ ID | Linker | GGGGSGGGGSGGGGSGGGGS |
| NO: 63 | | |
| SEQ ID | scFv (VH- | EVQLVESGGGLVKPGGSLRLSCAASGFTFSSFRMNWVRQAPGKGLEWVS |
| NO: 175 | linker-VL) | SISSSSSYIYYADSVKGRFTISRDNAKNSLYLQMNSLRAEDTAVYYCARW |
| | | LSYYGMDVWGQGTTVTVSSGGGGSGGGGSGGGGSGGGGSDIVMTQTPL |
| | | SLPVTPGEPASISCRSSQSLLDSDDGNTYLDWYLQKPGQSPQLLIYTLSFRA |
| | | SGVPDRFSGSGSGTDFTLKIRRVEAEDVGVYYCMQRIGFPITFGQGTRLEI |
| | | K |
| SEQ ID | DNA scFv | GAAGTGCAACTGGTGGAGAGCGGTGGAGGGCTTGTCAAGCCCGGAGG |
| NO: 176 | | ATCGCTGCGGCTGTCCTGTGCTGCGTCCGGGTTCACCTTCTCCTCGTTC |
| | | CGCATGAACTGGGTCAGACAGGCACCGGGAAAGGGCCTCGAATGGGT |
| | | GTCCTCAATCTCATCGTCCTCGTCCTACATCTACTACGCCGACTCCGTG |
| | | AAAGGCCGCTTCACCATCTCCCGGGACAACGCCAAGAACTCGCTGTAC |
| | | CTCCAAATGAATAGCCTCAGGGCGGAAGATACTGCTGTGTATTACTGC |
| | | GCACGCTGGCTTTCCTACTACGGCATGGACGTCTGGGGCCAAGGGACC |
| | | ACTGTGACCGTGTCTAGCGGAGGCGGAGGTTCAGGGGGCGGTGGATC |
| | | AGGCGGAGGAGGATCGGGGGGTGGTGGATCGGATATCGTGATGACCC |
| | | AGACTCCCCTGTCCCTGCCTGTGACTCCCGGAGAACCAGCCTCCATTTC |
| | | CTGCCGGTCCTCCCAGTCCCTGCTGGACAGCGACGACGGCAACACTTA |
| | | CCTGGACTGGTACTTGCAGAAGCCGGGCCAATCGCCTCAGCTGCTGAT |
| | | CTATACCCTGTCATTCCGGGCCTCAGGAGTGCCTGACCGCTTCTCGGG |
| | | ATCAGGGAGCGGGACCGATTTCACCCTGAAAATTAGGCGAGTGGAAG |
| | | CCGAGGACGTCGGAGTGTACTACTGCATGCAGCGCATCGGCTTCCCGA |
| | | TTACGTTTGGACAGGGTACCCGGCTTGAGATCAAG |
| SEQ ID | Full CAR | EVQLVESGGGLVKPGGSLRLSCAASGFTFSSFRMNWVRQAPGKGLEWVS |
| NO: 177 | amino acid | SISSSSSYIYYADSVKGRFTISRDNAKNSLYLQMNSLRAEDTAVYYCARW |
| | sequence | LSYYGMDVWGQGTTVTVSSGGGGSGGGGSGGGGSGGGGSDIVMTQTPL |
| | | SLPVTPGEPASISCRSSQSLLDSDDGNTYLDWYLQKPGQSPQLLIYTLSFRA |
| | | SGVPDRFSGSGSGTDFTLKIRRVEAEDVGVYYCMQRIGFPITFGQGTRLEI |
| | | KTTTPAPRPPTPAPTIASQPLSLRPEACRPAAGGAVHTRGLDFACDIYIWAP |
| | | LAGTCGVLLLSLVITLYCKRGRKKLLYIFKQPFMRPVQTTQEEDGCSCRFP |
| | | EEEEGGCELRVKFSRSADAPAYQQGQNQLYNELNLGRREEYDVLDKRRG |
| | | RDPEMGGKPRRKNPQEGLYNELQKDKMAEAYSEIGMKGERRRGKGHDG |
| | | LYQGLSTATKDTYDALHMQALPPR |
| SEQ ID | Full CAR | GAAGTGCAACTGGTGGAGAGCGGTGGAGGGCTTGTCAAGCCCGGAGG |
| NO: 178 | DNA | ATCGCTGCGGCTGTCCTGTGCTGCGTCCGGGTTCACCTTCTCCTCGTTC |
| | sequence | CGCATGAACTGGGTCAGACAGGCACCGGGAAAGGGCCTCGAATGGGT |
| | | GTCCTCAATCTCATCGTCCTCGTCCTACATCTACTACGCCGACTCCGTG |
| | | AAAGGCCGCTTCACCATCTCCCGGGACAACGCCAAGAACTCGCTGTAC |
| | | CTCCAAATGAATAGCCTCAGGGCGGAAGATACTGCTGTGTATTACTGC |
| | | GCACGCTGGCTTTCCTACTACGGCATGGACGTCTGGGGCCAAGGGACC |
| | | ACTGTGACCGTGTCTAGCGGAGGCGGAGGTTCAGGGGGCGGTGGATC |
| | | AGGCGGAGGAGGATCGGGGGGTGGTGGATCGGATATCGTGATGACCC |
| | | AGACTCCCCTGTCCCTGCCTGTGACTCCCGGAGAACCAGCCTCCATTTC |
| | | CTGCCGGTCCTCCCAGTCCCTGCTGGACAGCGACGACGGCAACACTTA |
| | | CCTGGACTGGTACTTGCAGAAGCCGGGCCAATCGCCTCAGCTGCTGAT |
| | | CTATACCCTGTCATTCCGGGCCTCAGGAGTGCCTGACCGCTTCTCGGG |
| | | ATCAGGGAGCGGGACCGATTTCACCCTGAAAATTAGGCGAGTGGAAG |
| | | CCGAGGACGTCGGAGTGTACTACTGCATGCAGCGCATCGGCTTCCCGA |
| | | TTACGTTTGGACAGGGTACCCGGCTTGAGATCAAGACCACTACCCCAG |
| | | CACCGAGGCCACCCACCCCGGCTCCTACCATCGCCTCCCAGCCTCTGT |
| | | CCCTGCGTCCGGAGGCATGTAGACCCGCAGCTGGTGGGGCCGTGCATA |
| | | CCCGGGGTCTTGACTTCGCCTGCGATATCTACATTTGGGCCCCTCTGGC |
| | | TGGTACTTGCGGGGTCCTGCTGCTTTCACTCGTGATCACTCTTTACTGT |
| | | AAGCGCGGTCGGAAGAAGCTGCTGTACATCTTTAAGCAACCCTTCATG |
| | | AGGCCTGTGCAGACTACTCAAGAGGAGGACGGCTGTTCATGCCGGTTC |
| | | CCAGAGGAGGAGGAAGGCGGCTGCGAACTGCGCGTGAAATTCAGCCG |
| | | CAGCGCAGATGCTCCAGCCTACCAGCAGGGGCAGAACCAGCTCTACA |
| | | ACGAACTCAATCTTGGTCGGAGAGAGGAGTACGACGTGCTGGACAAG |
| | | CGGAGAGGACGGGACCCAGAAATGGGCGGGAAGCCGCGCAGAAAGA |
| | | ATCCCCAAGAGGGCCTGTACAACGAGCTCCAAAAGGATAAGATGGCA |
| | | GAAGCCTATAGCGAGATTGGTATGAAAGGGGAACGCAGAAGAGGCAA |
| | | AGGCCACGACGGACTGTACCAGGGACTCAGCACCGCCACCAAGGACA |
| | | CCTATGACGCTCTTCACATGCAGGCCCTGCCGCCTCGG |

TABLE 13
Kabat CDRs of exemplary hybridoma-derived anti-BCMA molecules

| Kabat | HCDR1 | HCDR2 | HCDR3 | LCDR1 | LCDR2 | LCDR3 |
|---|---|---|---|---|---|---|
| Hy03 | GFWMS | NIKQDGSEK | ALDYYGMDV | RSSQSLLDS | TLSYRAS | TQRLEFPSIT |
| | (SEQ ID | YYVDSVRG | (SEQ ID | DDGNTYLD | (SEQ ID | (SEQ ID |
| | NO: 137) | (SEQ ID | NO: 139) | (SEQ ID | NO: 148) | NO: 149) |
| | | NO: 138) | | NO: 147) | | |
| Hy52 | SFRMN | SISSSSSYI | WLSYYGMDV | RSSQSLLDS | TLSFRAS | MQRIGFPIT |
| | (SEQ ID | YYADSVKG | (SEQ ID | DDGNTYLD | (SEQ ID | (SEQ ID |
| | NO: 160) | (SEQ ID | NO: 162) | (SEQ ID | NO: 170) | NO: 171) |
| | | NO: 161) | | NO: 147) | | |
| Consensus | X₁FX₂MX₃, | X₁IX₂X₃X₄X₅ | X₁LX₂YYGMDV, | RSSQSLLDS | TLSXRAS, | X₁QRX₂X₃FP |
| | wherein | SX₆X₇YYX₈DS | wherein X₁ | DDGNTYLD | wherein | X₄IT, |
| | X₁ is G or | VX₉G, wherein | is A or W; | (SEQ ID | X is Y or | wherein |
| | S; X₂ is W | X₁ is N or S; | and X₂ is | NO: 147) | F (SEQ ID | X₁ is T or |
| | or R; and | X₂ is K or S; | D or S | | NO: 182) | M; X₂ is L |
| | X₃ is S or | X₃ is Q or S; | (SEQ ID | | | or I; X₃ |
| | N (SEQ ID | X₄ is D or S; | NO: 181) | | | is E or G; |
| | NO: 179) | X₅ is G or S; | | | | and X₄ is |
| | | X₆ is E or Y; | | | | S or absent |
| | | X₇ is K or I; | | | | (SEQ ID |
| | | X₈ is V or A; | | | | NO: 183) |
| | | and X₉ is R | | | | |
| | | or K (SEQ ID | | | | |
| | | NO: 180) | | | | |

TABLE 14
Chothia CDRs of exemplary hybridoma-derived anti-BCMA molecules

| Chothia | HCDR1 | HCDR2 | HCDR3 | LCDR1 | LCDR2 | LCDR3 |
|---|---|---|---|---|---|---|
| Hy03 | GFTFSGF | KQDGSE | ALDYYGMDV | SQSLLDS | TLS | RLEFPSI |
| | (SEQ ID | (SEQ ID | (SEQ ID | DDGNTY | (SEQ ID | (SEQ ID |
| | NO: 140) | NO: 141) | NO: 139) | (SEQ ID | NO: 151) | NO: 152) |
| | | | | NO: 150) | | |
| Hy52 | GFTFSSF | SSSSSY | WLSYYGMDV | SQSLLDS | TLS | RIGFPI |
| | (SEQ ID | (SEQ ID | (SEQ ID | DDGNTY | (SEQ ID | (SEQ ID |
| | NO: 163) | NO: 164) | NO: 162) | (SEQ ID | NO: 151) | NO: 172) |
| | | | | NO: 150) | | |
| Consensus | GFTFSXF, | X₁X₂X₃X₄SX₅, | X₁LX₂YYGMDV, | SQSLLDS | TLS | RX1X2FPX₃I, |
| | wherein | wherein X₁ | wherein X₁ | DDGNTY | (SEQ ID | wherein |
| | X is G | is K or S; | is A or W; | (SEQ ID | NO: 151) | X₁ is L or |
| | or S | X₂ is Q or | and X₂ is | NO: 150) | | I; X₂ is E |
| | (SEQ ID | S; X₃ is D | D or S | | | or G; and |
| | NO: 184) | or S; X₄ is | (SEQ ID | | | X₃ is S or |
| | | G or S; and | NO: 181) | | | absent |
| | | X₅ is E or | | | | (SEQ ID |
| | | Y (SEQ ID | | | | NO: 186) |
| | | NO: 185) | | | | |

TABLE 15
IMGT CDRs of exemplary hybridoma-derived anti-BCMA molecules

| IMGT | HCDR1 | HCDR2 | HCDR3 | LCDR1 | LCDR2 | LCDR3 |
|---|---|---|---|---|---|---|
| Hy03 | GFTFSGFW | IKQDGSEK | ARALDYYGMDV | QSLLDS | TLS | TQRLEFPSIT |
| | (SEQ ID | (SEQ ID | (SEQ ID | DDGNTY | (SEQ ID | (SEQ ID |
| | NO: 142) | NO: 143) | NO: 144) | (SEQ ID | NO: 151) | NO: 149) |
| | | | | NO: 153) | | |
| Hy52 | GFTFSSFR | ISSSSSYI | ARWLSYYGMDV | QSLLDS | TLS | MQRIGFPIT |
| | (SEQ ID | (SEQ ID | (SEQ ID | DDGNTY | (SEQ ID | (SEQ ID |
| | NO: 165) | NO: 166) | NO: 167) | (SEQ ID | NO: 151) | NO: 171) |
| | | | | NO: 153) | | |
| Consensus | GFTFSX₁FX₂, | IX₁X₂X₃X₄SX₅X₆, | ARX₁LX₂YYGMDV, | QSLLDS | TLS | X₁QRX₂X₃FP |
| | wherein X₁ | wherein X₁ is | wherein X₁ is | DDGNTY | (SEQ ID | X₄IT, |
| | is G or S; | K or S; X₂ is | A or W; and | (SEQ ID | NO: 151) | wherein X₁ |
| | and X₂ is | Q or S; X₃ is | X₂ is D or S | NO: 153) | | is T or M; |
| | W or R | D or S; X₄ is | (SEQ ID | | | X₂ is L or |
| | (SEQ ID | G or S; X₅ is | NO: 189) | | | I; X₃ is E |
| | NO: 187) | E or Y; and | | | | or G; and |
| | | X₆ is K or I | | | | X₄ is S or |
| | | (SEQ ID | | | | absent |
| | | NO: 188) | | | | (SEQ ID |
| | | | | | | NO: 183) |

TABLE 20
Amino acid and nucleic acid sequences of exemplary anti-BCMA molecules

| SEQ | Name/ | |
|---|---|---|
| ID NO | Description | Sequence |
| duBCMA.4 | | |
| SEQ ID | HCDR1 | NHGMS |
| NO: 231 | (Kabat) | |
| SEQ ID | HCDR2 | GIVYSGSTYYAASVKG |
| NO: 232 | (Kabat) | |
| SEQ ID | HCDR3 | HGGESDV |
| NO: 233 | (Kabat) | |
| SEQ ID | HCDR1 | GFALSNH |
| NO: 234 | (Chothia) | |
| SEQ ID | HCDR2 | VYSGS |
| NO: 235 | (Chothia) | |
| SEQ ID | HCDR3 | HGGESDV |
| NO: 233 | (Chothia) | |
| SEQ ID | HCDR1 | GFALSNHG |
| NO: 236 | (IMGT) | |
| SEQ ID | HCDR2 | IVYSGST |
| NO: 237 | (IMGT) | |
| SEQ ID | HCDR3 | SAHGGESDV |
| NO: 238 | (IMGT) | |
| SEQ ID | VH | EVQLVESGGGLVQPGGSLRLSCAVSGFALSNHGMSWVRRAPGKGLEWV |
| NO: 239 | | SGIVYSGSTYYAASVKGRFTISRDNSRNTLYLQMNSLRPEDTAIYYCS |
| | | AHGGESDVWGQGTTVTVSS |
| SEQ ID | DNA VH | GAAGTGCAATTGGTGGAATCAGGGGGAGGACTTGTGCAGCCTGGAGGA |
| NO: 262 | | TCGCTGAGACTGTCATGTGCCGTGTCCGGCTTTGCCCTGTCCAACCAC |
| | | GGGATGTCCTGGGTCCGCCGCGCGCCTGGAAAGGGCCTCGAATGGGTG |
| | | TCGGGTATTGTGTACAGCGGTAGCACCTACTATGCCGCATCCGTGAAG |
| | | GGGAGATTCACCATCAGCCGGGACAACTCCAGGAACACTCTGTACCTC |
| | | CAAATGAATTCGCTGAGGCCAGAGGACACTGCCATCTACTACTGCTCC |
| | | GCGCATGGCGGAGAGTCCGACGTCTGGGGACAGGGGACCACCGTGACC |
| | | GTGTCTAGC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPYT |
| NO: 240 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPY |
| NO: 241 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPYT |
| NO: 240 | (IMGT) | |
| SEQ ID | VL | DIQLTQSPSSLSASVGDRVTITCRASQSISSYLNWYQQKPGKAPKLLI |
| NO: 242 | | YAASSLQSGVPSRFSGSGSGTDFTLTISSLQPEDFATYYCQQSYSTPY |
| | | TFGQGTKVEIK |
| SEQ ID | DNA VL | GACATCCAGCTCACCCAGTCCCCGAGCTCGCTGTCCGCCTCCGTGGGA |
| NO: 263 | | GATCGGGTCACCATCACGTGCCGCGCCAGCCAGTCGATTTCCTCCTAC |
| | | CTGAACTGGTACCAACAGAAGCCCGGAAAAGCCCCGAAGCTTCTCATC |
| | | TACGCCGCCTCGAGCCTGCAGTCAGGAGTGCCCTCACGGTTCTCCGGC |
| | | TCCGGTTCCGGTACTGATTTCACCCTGACCATTTCCTCCCTGCAACCG |
| | | GAGGACTTCGCTACTTACTACTGCCAGCAGTCGTACTCCACCCCCTAC |
| | | ACTTTCGGACAAGGCACCAAGGTCGAAATCAAG |
| SEQ ID | Linker | ASGGGGSGGGGSGGGGS |
| NO: 243 | | |
| SEQ ID | scFv (VH- | EVQLVESGGGLVQPGGSLRLSCAVSGFALSNHGMSWVRRAPGKGLEWV |
| NO: 200 | linker-VL) | SGIVYSGSTYYAASVKGRFTISRDNSRNTLYLQMNSLRPEDTAIYYCS |
| | | AHGGESDVWGQGTTVTVSSASGGGGSGGGGSGGGGSDIQLTQSPSSLS |
| | | ASVGDRVTITCRASQSISSYLNWYQQKPGKAPKLLIYAASSLQSGVPS |
| | | RFSGSGSGTDFTLTISSLQPEDFATYYCQQSYSTPYTFGQGTKVEIK |
| SEQ ID | DNA scFv | GAAGTGCAATTGGTGGAATCAGGGGGAGGACTTGTGCAGCCTGGAGGA |
| NO: 201 | | TCGCTGAGACTGTCATGTGCCGTGTCCGGCTTTGCCCTGTCCAACCAC |
| | | GGGATGTCCTGGGTCCGCCGCGCGCCTGGAAAGGGCCTCGAATGGGTG |
| | | TCGGGTATTGTGTACAGCGGTAGCACCTACTATGCCGCATCCGTGAAG |
| | | GGGAGATTCACCATCAGCCGGGACAACTCCAGGAACACTCTGTACCTC |
| | | CAAATGAATTCGCTGAGGCCAGAGGACACTGCCATCTACTACTGCTCC |
| | | GCGCATGGCGGAGAGTCCGACGTCTGGGGACAGGGGACCACCGTGACC |
| | | GTGTCTAGCGCGTCCGGCGGAGGCGGCAGCGGGGGTGGTGGTTCAGGG |
| | | GGCGGCGGATCGGACATCCAGCTCACCCAGTCCCCGAGCTCGCTGTCC |
| | | GCCTCCGTGGGAGATCGGGTCACCATCACGTGCCGCGCCAGCCAGTCG |
| | | ATTTCCTCCTACCTGAACTGGTACCAACAGAAGCCCGGAAAAGCCCCG |
| | | AAGCTTCTCATCTACGCCGCCTCGAGCCTGCAGTCAGGAGTGCCCTCA |
| | | CGGTTCTCCGGCTCCGGTTCCGGTACTGATTTCACCCTGACCATTTCC |
| | | TCCCTGCAACCGGAGGACTTCGCTACTTACTACTGCCAGCAGTCGTAC |
| | | TCCACCCCCTACACTTTCGGACAAGGCACCAAGGTCGAAATCAAG |
| SEQ ID | Full CAR | EVQLVESGGGLVQPGGSLRLSCAVSGFALSNHGMSWVRRAPGKGLEWV |
| NO: 230 | amino acid | SGIVYSGSTYYAASVKGRFTISRDNSRNTLYLQMNSLRPEDTAIYYCS |
| | sequence | AHGGESDVWGQGTTVTVSSASGGGGSGGGGSGGGGSDIQLTQSPSSLS |
| | | ASVGDRVTITCRASQSISSYLNWYQQKPGKAPKLLIYAASSLQSGVPS |
| | | RFSGSGSGTDFTLTISSLQPEDFATYYCQQSYSTPYTFGQGTKVEIKT |
| | | TTPAPRPPTPAPTIASQPLSLRPEACRPAAGGAVHTRGLDFACDIYIW |
| | | APLAGTCGVLLLSLVITLYCKRGRKKLLYIFKQPFMRPVQTTQEEDGC |
| | | SCRFPEEEEGGCELRVKFSRSADAPAYQQGQNQLYNELNLGRREEYDV |
| | | LDKRRGRDPEMGGKPRRKNPQEGLYNELQKDKMAEAYSEIGMKGERRR |
| | | GKGHDGLYQGLSTATKDTYDALHMQALPPR |

TABLE 26
CDRs of exemplary anti-BCMA molecules

| duBCMA.4 | HCDR1 | HCDR2 | HCDR3 | LCDR1 | LCDR2 | LCDR3 |
|---|---|---|---|---|---|---|
| Kabat | SEQ ID | SEQ ID | SEQ ID | SEQ ID | SEQ ID | SEQ ID |
| | NO: 231 | NO: 232 | NO: 233 | NO: 54 | NO: 55 | NO: 240 |
| Chothia | SEQ ID | SEQ ID | SEQ ID | SEQ ID | SEQ ID | SEQ ID |
| | NO: 234 | NO: 235 | NO: 233 | NO: 57 | NO: 58 | NO: 241 |
| IMGT | SEQ ID | SEQ ID | SEQ ID | SEQ ID | SEQ ID | SEQ ID |
| | NO: 236 | NO: 237 | NO: 238 | NO: 60 | NO: 58 | NO: 240 |

In some embodiments, the human anti-BCMA binding domain comprises a HC CDR1, HC CDR2, HC CDR3, LC CDR1, LC CDR2, and LC CDR3.

In certain embodiments, the CAR molecule described herein or the anti-BCMA binding domain described herein includes:
(1) one, two, or three light chain (LC) CDRs chosen from:
   (i) a LC CDR1 of SEQ ID NO: 54, LC CDR2 of SEQ ID NO: 55 and LC CDR3 of SEQ ID NO: 56; and/or
(2) one, two, or three heavy chain (HC) CDRs from one of the following:
   (i) a HC CDR1 of SEQ ID NO: 44, HC CDR2 of SEQ ID NO: 45 and HC CDR3 of SEQ ID NO: 84; (ii) a HC CDR1 of SEQ ID NO: 44, HC CDR2 of SEQ ID NO: 45 and HC CDR3 of SEQ ID NO: 46; (iii) a HC CDR1 of SEQ ID NO: 44, HC CDR2 of SEQ ID NO: 45 and HC CDR3 of SEQ ID NO: 68; or (iv) a HC CDR1 of SEQ ID NO: 44, HC CDR2 of SEQ ID NO: 45 and HC CDR3 of SEQ ID NO: 76.

In certain embodiments, the CAR molecule described herein or the anti-BCMA binding domain described herein includes:
(1) one, two, or three light chain (LC) CDRs from one of the following:
   (i) a LC CDR1 of SEQ ID NO: 95, LC CDR2 of SEQ ID NO: 131 and LC CDR3 of SEQ ID NO: 132; (ii) a LC CDR1 of SEQ ID NO: 95, LC CDR2 of SEQ ID NO: 96 and LC CDR3 of SEQ ID NO: 97; (iii) a LC CDR1 of SEQ ID NO: 95, LC CDR2 of SEQ ID NO: 114 and LC CDR3 of SEQ ID NO: 115; or (iv) a LC CDR1 of SEQ ID NO: 95, LC CDR2 of SEQ ID NO: 114 and LC CDR3 of SEQ ID NO: 97; and/or
(2) one, two, or three heavy chain (HC) CDRs from one of the following:
   (i) a HC CDR1 of SEQ ID NO: 86, HC CDR2 of SEQ ID NO: 130 and HC CDR3 of SEQ ID NO: 88; (ii) a HC CDR1 of SEQ ID NO: 86, HC CDR2 of SEQ ID NO: 87 and HC CDR3 of SEQ ID NO: 88; or (iii) a HC CDR1 of SEQ ID NO: 86, HC CDR2 of SEQ ID NO: 109 and HC CDR3 of SEQ ID NO: 88.

In certain embodiments, the CAR molecule described herein or the anti-BCMA binding domain described herein includes:
(1) one, two, or three light chain (LC) CDRs from one of the following:
   (i) a LC CDR1 of SEQ ID NO: 147, LC CDR2 of SEQ ID NO: 182 and LC CDR3 of SEQ ID NO: 183; (ii) a LC CDR1 of SEQ ID NO: 147, LC CDR2 of SEQ ID NO: 148 and LC CDR3 of SEQ ID NO: 149; or (iii) a LC CDR1 of SEQ ID NO: 147, LC CDR2 of SEQ ID NO: 170 and LC CDR3 of SEQ ID NO: 171; and/or
(2) one, two, or three heavy chain (HC) CDRs from one of the following:
   (i) a HC CDR1 of SEQ ID NO: 179, HC CDR2 of SEQ ID NO: 180 and HC CDR3 of SEQ ID NO: 181; (ii) a HC CDR1 of SEQ ID NO: 137, HC CDR2 of SEQ ID NO: 138 and HC CDR3 of SEQ ID NO: 139; or (iii) a HC CDR1 of SEQ ID NO: 160, HC CDR2 of SEQ ID NO: 161 and HC CDR3 of SEQ ID NO: 162.

In some embodiments, the HC CDR1, HC CDR2, HC CDR3, LC CDR1, LC CDR2, and LC CDR3 comprise the amino acid sequences of SEQ ID NOs: 44, 45, 84, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 46, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 68, 54, 55, and 56, respectively. In some embodiments, the HC CDR1, HC CDR2, HC CDR3, LC CDR1, LC CDR2, and LC CDR3 comprise the amino acid sequences of SEQ ID NOs: 44, 45, 76, 54, 55, and 56, respectively.

In some embodiments, the HC CDR1, HC CDR2, HC CDR3, LC CDR1, LC CDR2, and LC CDR3 comprise the amino acid sequences of SEQ ID NOs: 47, 48, 84, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 46, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 68, 57, 58, and 59, respectively. In some embodiments, the HC CDR1, HC CDR2, HC CDR3, LC CDR1, LC CDR2, and LC CDR3 comprise the amino acid sequences of SEQ ID NOs: 47, 48, 76, 57, 58, and 59, respectively.

In some embodiments, the HC CDR1, HC CDR2, HC CDR3, LC CDR1, LC CDR2, and LC CDR3 comprise the amino acid sequences of SEQ ID NOs: 49, 50, 85, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 51, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 69, 60, 58, and 56, respectively. In some embodiments, the HC CDR1, HC CDR2, HC CDR3, LC CDR1, LC CDR2, and LC CDR3 comprise the amino acid sequences of SEQ ID NOs: 49, 50, 77, 60, 58, and 56, respectively.

In some embodiments, the human anti-BCMA binding domain comprises a scFv comprising a VH (for example, a VH described herein) and VL (for example, a VL described herein). In some embodiments, the VH is attached to the VL via a linker, for example, a linker described herein, for example, a linker described in Table 1. In some embodiments, the human anti-BCMA binding domain comprises a (Gly4-Ser)n linker, wherein n is 1, 2, 3, 4, 5, or 6, preferably 3 or 4 (SEQ ID NO: 26). The light chain variable region and heavy chain variable region of a scFv can be, for example, in any of the following orientations: light chain variable region-linker-heavy chain variable region or heavy chain variable region-linker-light chain variable region.

In some embodiments, the anti-BCMA binding domain is a fragment, for example, a single chain variable fragment (scFv). In some embodiments, the anti-BCMA binding domain is a Fv, a Fab, a (Fab′)2, or a bi-functional (for example bi-specific) hybrid antibody (for example, Lanzavecchia et al., Eur. J. Immunol. 17, 105 (1987)). In some embodiments, the antibodies and fragments thereof of the invention binds a BCMA protein with wild-type or enhanced affinity.

In some instances, scFvs can be prepared according to method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (for example, a Ser-Gly linker) with an optimized length and/or amino acid composition. The linker length can greatly affect how the variable regions of a scFv fold and interact. In fact, if a short polypeptide linker is employed (for example, between 5-10 amino acids) intrachain folding is prevented. Interchain folding is also required to bring the two variable regions together to form a functional epitope binding site. For examples of linker orientation and size see, for example, Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, is incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In some embodiments, the linker sequence comprises sets of glycine and serine repeats such as (Gly₄Ser)n, where n is a positive integer equal to or greater than 1 (SEQ ID NO: 25). In some embodiments, the linker can be (Gly₄Ser)₄ (SEQ ID NO: 27) or (Gly₄Ser)₃(SEQ ID NO: 28). Variation in the linker length may retain or enhance activity, giving rise to superior efficacy in activity studies.

### CD20 Car

In some embodiments, the CAR-expressing cell described herein is a CD20 CAR-expressing cell (for example, a cell expressing a CAR that binds to human CD20). In some embodiments, the CD20 CAR-expressing cell includes an antigen binding domain according to WO2016164731 and WO2018067992, incorporated herein by reference. Exemplary CD20-binding sequences or CD20 CAR sequences are disclosed in, for example, Tables 1-5 of WO2018067992. In some embodiments, the CD20 CAR comprises a CDR, variable region, scFv, or full-length sequence of a CD20 CAR disclosed in WO2018067992 or WO2016164731.

### CD22 Car

In some embodiments, the CAR-expressing cell described herein is a CD22 CAR-expressing cell (for example, a cell expressing a CAR that binds to human CD22). In some embodiments, the CD22 CAR-expressing cell includes an antigen binding domain according to WO2016164731 and WO2018067992, incorporated herein by reference. Exemplary CD22-binding sequences or CD22 CAR sequences are disclosed in, for example, Tables 6A, 6B, 7A, 7B, 7C, 8A, 8B, 9A, 9B, 10A, and 10B of WO2016164731 and Tables 6-10 of WO2018067992. In some embodiments, the CD22 CAR sequences comprise a CDR, variable region, scFv or full-length sequence of a CD22 CAR disclosed in WO2018067992 or WO2016164731.

In embodiments, the CAR molecule comprises an antigen binding domain that binds to CD22 (CD22 CAR). In some embodiments, the antigen binding domain targets human CD22. In some embodiments, the antigen binding domain includes a single chain Fv sequence as described herein.

The sequences of human CD22 CAR are provided below. In some embodiments, a human CD22 CAR is CAR22-65.

Human CD22 CAR scFv sequence
(SEQ ID NO: 285)
EVQLQQSGPGLVKPSQTLSLTCAISGDSMLSNSDTWNWIRQSPSRGLEWL
GRTYHRSTWYDDYASSVRGRVSINVDTSKNQYSLQLNAVTPEDTGVYYCA
RVRLQDGNSWSDAFDVWGQGTMVTVSSGGGGSGGGGSGGGGSQSALTQPA
SASGSPGQSVTISCTGTSSDVGGYNYVSWYQQHPGKAPKLMIYDVSNRPS
GVSNRFSGSKSGNTASLTISGLQAEDEADYYCSSYTSSSTLYVFGTGTQL
TVL
Human CD22 CAR heavy chain variable region
(SEQ ID NO 286)
EVQLQQSGPGLVKPSQTLSLTCAISGDSMLSNSDTWNWIRQSPSRGLEWL
GRTYHRSTWYDDYASSVRGRVSINVDTSKNQYSLQLNAVTPEDTGVYYCA
RVRLQDGNSWSDAFDVWGQGTMVTVSS
Human CD22 CAR light chain variable region
(SEQ ID NO 287)
QSALTQPASASGSPGQSVTISCTGTSSDVGGYNYVSWYQQHPGKAPKLMI
YDVSNRPSGVSNRFSGSKSGNTASLTISGLQAEDEADYYCSSYTSSSTLY
VFGTGTQLTVL

TABLE 16
Heavy Chain Variable Domain CDRs of CD22 CAR (CAR22-65)

| | | SEQ | | SEQ | | SEQ |
|---|---|---|---|---|---|---|
| Candidate | HCDR1 | ID NO: | HCDR2 | ID NO: | HCDR3 | ID NO: |
| CAR22-65 | GDSMLSN | 288 | RTYHRSTWY | 290 | VRLQDGNS | 291 |
| Combined | SDTWN | | DDYASSVRG | | WSDAFDV | |
| CAR22-65 | SNSDTWN | 289 | RTYHRSTWY | 290 | VRLQDGNS | 291 |
| Kabat | | | DDYASSVRG | | WSDAFDV | |

TABLE 17
Light Chain Variable Domain CDRs of CD22 CAR (CAR22-65).
The LC CDR sequences in this table have the
same sequence under the Kabat or combined definitions.

| | | SEQ | | SEQ | | SEQ |
|---|---|---|---|---|---|---|
| Candidate | LCDR1 | ID NO: | LCDR2 | ID NO: | LCDR3 | ID NO: |
| CAR22-65 | TGTSSDV | 95 | DVSNRPS | 96 | SSYTSS | 97 |
| Combined | GGYNYVS | | | | STLYV | |

In some embodiments, the antigen binding domain comprises a HC CDR1, a HC CDR2, and a HC CDR3 of any heavy chain binding domain amino acid sequences listed in Table 16. In embodiments, the antigen binding domain further comprises a LC CDR1, a LC CDR2, and a LC CDR3. In embodiments, the antigen binding domain comprises a LC CDR1, a LC CDR2, and a LC CDR3 amino acid sequences listed in Table 17.

In some embodiments, the antigen binding domain comprises one, two or all of LC CDR1, LC CDR2, and LC CDR3 of any light chain binding domain amino acid sequences listed in Table 17, and one, two or all of HC CDR1, HC CDR2, and HC CDR3 of any heavy chain binding domain amino acid sequences listed in Table 16.

In some embodiments, the CDRs are defined according to the Kabat numbering scheme, the Chothia numbering scheme, or a combination thereof.

The order in which the VL and VH domains appear in the scFv can be varied (i.e., VL-VH, or VH-VL orientation), and where any of one, two, three or four copies of the “G4S” subunit (SEQ ID NO: 25), in which each subunit comprises the sequence GGGGS (SEQ ID NO: 25) (for example, (G4S)₃ (SEQ ID NO: 28) or (G4S)₄ (SEQ ID NO: 27)), can connect the variable domains to create the entirety of the scFv domain. Alternatively, the CAR construct can include, for example, a linker including the sequence GSTSGSGKPGSGEGSTKG (SEQ ID NO: 43). Alternatively, the CAR construct can include, for example, a linker including the sequence LAEAAAK (SEQ ID NO: 308). In some embodiments, the CAR construct does not include a linker between the VL and VH domains.

These clones all contained a Q/K residue change in the signal domain of the co-stimulatory domain derived from CD3zeta chain.

### EGFR Car

In some embodiments, the CAR-expressing cell described herein is an EGFR CAR-expressing cell (for example, a cell expressing a CAR that binds to human EGFR). In some embodiments, the CAR-expressing cell described herein is an EGFRvIII CAR-expressing cell (for example, a cell expressing a CAR that binds to human EGFRvIII). Exemplary EGFRvIII CARs can include sequences disclosed in WO2014/130657, for example, Table 2 of WO2014/130657, incorporated herein by reference.

Exemplary EGFRvIII-binding sequences or EGFR CAR sequences may comprise a CDR, a variable region, an scFv, or a full-length CAR sequence of a EGFR CAR disclosed in WO2014/130657.

### Mesothelin Car

In some embodiments, the CAR-expressing cell described herein is a mesothelin CAR-expressing cell (for example, a cell expressing a CAR that binds to human mesothelin). Exemplary mesothelin CARs can include sequences disclosed in WO2015090230 and WO2017112741, for example, Tables 2, 3, 4, and 5 of WO2017112741, incorporated herein by reference.

Other exemplary Cars

In other embodiments, the CAR-expressing cells can specifically bind to CD123, for example, can include a CAR molecule (for example, any of the CAR1 to CAR8), or an antigen binding domain according to Tables 1-2 of WO 2014/130635, incorporated herein by reference. The amino acid and nucleotide sequences encoding the CD123 CAR molecules and antigen binding domains (for example, including one, two, three VH CDRs; and one, two, three VL CDRs according to Kabat or Chothia), are specified in WO 2014/130635. In other embodiments, the CAR-expressing cells can specifically bind to CD123, for example, can include a CAR molecule (for example, any of the CAR123-1 to CAR123-4 and hzCAR123-1 to hzCAR123-32), or an antigen binding domain according to Tables 2, 6, and 9 of WO2016/028896, incorporated herein by reference. The amino acid and nucleotide sequences encoding the CD123 CAR molecules and antigen binding domains (for example, including one, two, three VH CDRs; and one, two, three VL CDRs according to Kabat or Chothia), are specified in WO2016/028896.

In some embodiments, the CAR molecule comprises a CLL1 CAR described herein, for example, a CLL1 CAR described in US2016/0051651A1, incorporated herein by reference. In embodiments, the CLL1 CAR comprises an amino acid, or has a nucleotide sequence shown in US2016/0051651A1, incorporated herein by reference. In other embodiments, the CAR-expressing cells can specifically bind to CLL-1, for example, can include a CAR molecule, or an antigen binding domain according to Table 2 of WO2016/014535, incorporated herein by reference. The amino acid and nucleotide sequences encoding the CLL-1 CAR molecules and antigen binding domains (for example, including one, two, three VH CDRs; and one, two, three VL CDRs according to Kabat or Chothia), are specified in WO2016/014535.

In some embodiments, the CAR molecule comprises a CD33 CAR described herein, e.g. a CD33 CAR described in US2016/0096892A1, incorporated herein by reference. In embodiments, the CD33 CAR comprises an amino acid, or has a nucleotide sequence shown in US2016/0096892A1, incorporated herein by reference. In other embodiments, the CAR-expressing cells can specifically bind to CD33, for example, can include a CAR molecule (for example, any of CAR33-1 to CAR-33-9), or an antigen binding domain according to Table 2 or 9 of WO2016/014576, incorporated herein by reference. The amino acid and nucleotide sequences encoding the CD33 CAR molecules and antigen binding domains (for example, including one, two, three VH CDRs; and one, two, three VL CDRs according to Kabat or Chothia), are specified in WO2016/014576.

In some embodiments, the antigen binding domain comprises one, two three (for example, all three) heavy chain CDRs, HC CDR1, HC CDR2 and HC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference), and/or one, two, three (for example, all three) light chain CDRs, LC CDR1, LC CDR2 and LC CDR3, from an antibody described herein (for example, an antibody described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference). In some embodiments, the antigen binding domain comprises a heavy chain variable region and/or a variable light chain region of an antibody listed above.

In embodiments, the antigen binding domain is an antigen binding domain described in WO2015/142675, US-2015-0283178-A1, US-2016-0046724-A1, US2014/0322212A1, US2016/0068601A1, US2016/0051651A1, US2016/0096892A1, US2014/0322275A1, or WO2015/090230, incorporated herein by reference.

In embodiments, the antigen binding domain targets BCMA and is described in US-2016-0046724-A1. In embodiments, the antigen binding domain targets CD19 and is described in US-2015-0283178-A1. In embodiments, the antigen binding domain targets CD123 and is described in US2014/0322212A1, US2016/0068601A1. In embodiments, the antigen binding domain targets CLL1 and is described in US2016/0051651A1. In embodiments, the antigen binding domain targets CD33 and is described in US2016/0096892A1.

Exemplary target antigens that can be targeted using the CAR-expressing cells, include, but are not limited to, CD19, CD123, EGFRvIII, CD33, mesothelin, BCMA, and GFR ALPHA-4, among others, as described in, for example, WO2014/153270, WO 2014/130635, WO2016/028896, WO 2014/130657, WO2016/014576, WO 2015/090230, WO2016/014565, WO2016/014535, and WO2016/025880, each of which is herein incorporated by reference in its entirety.

In other embodiments, the CAR-expressing cells can specifically bind to GFR ALPHA-4, for example, can include a CAR molecule, or an antigen binding domain according to Table 2 of WO2016/025880, incorporated herein by reference. The amino acid and nucleotide sequences encoding the GFR ALPHA-4 CAR molecules and antigen binding domains (for example, including one, two, three VH CDRs; and one, two, three VL CDRs according to Kabat or Chothia), are specified in WO2016/025880.

In some embodiments, the antigen binding domain of any of the CAR molecules described herein (for example, any of CD19, CD123, EGFRvIII, CD33, mesothelin, BCMA, and GFR ALPHA-4) comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antigen binding domain listed above. In some embodiments, the antigen binding domain comprises a heavy chain variable region and/or a variable light chain region of an antibody listed or described above.

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the tumor antigen is a tumor antigen described in International Application WO2015/142675, filed Mar. 13, 2015, which is herein incorporated by reference in its entirety. In some embodiments, the tumor antigen is chosen from one or more of: CD19; CD123; CD22; CD30; CD171; CS-1 (also referred to as CD2 subset 1, CRACC, SLAMF7, CD319, and 19A24); C-type lectin-like molecule-1 (CLL-1 or CLECL1); CD33; epidermal growth factor receptor variant III (EGFRvIII); ganglioside G2 (GD2); ganglioside GD3 (aNeu5Ac(2-8)aNeu5Ac(2-3)bD)Galp(1-4)bDGlcp(1-1)Cer); TNF receptor family member B cell maturation (BCMA); Tn antigen ((Tn Ag) or (GalNAcα-Ser/Thr)); prostate-specific membrane antigen (PSMA); Receptor tyrosine kinase-like orphan receptor 1 (ROR1); Fms-Like Tyrosine Kinase 3 (FLT3); Tumor-associated glycoprotein 72 (TAG72); CD38; CD44v6; Carcinoembryonic antigen (CEA); Epithelial cell adhesion molecule (EPCAM); B7H3 (CD276); KIT (CD117); Interleukin-13 receptor subunit alpha-2 (IL-13Ra2 or CD213A2); Mesothelin; Interleukin 11 receptor alpha (IL-11Ra); prostate stem cell antigen (PSCA); Protease Serine 21 (Testisin or PRSS21); vascular endothelial growth factor receptor 2 (VEGFR2); Lewis(Y) antigen; CD24; Platelet-derived growth factor receptor beta (PDGFR-beta); Stage-specific embryonic antigen-4 (SSEA-4); CD20; Folate receptor alpha; Receptor tyrosine-protein kinase ERBB2 (Her2/neu); Mucin 1, cell surface associated (MUC1); epidermal growth factor receptor (EGFR); neural cell adhesion molecule (NCAM); Prostase; prostatic acid phosphatase (PAP); elongation factor 2 mutated (ELF2M); Ephrin B2; fibroblast activation protein alpha (FAP); insulin-like growth factor 1 receptor (IGF-1 receptor), carbonic anhydrase IX (CAIX); Proteasome (Prosome, Macropain) Subunit, Beta Type, 9 (LMP2); glycoprotein 100 (gp100); oncogene fusion protein consisting of breakpoint cluster region (BCR) and Abelson murine leukemia viral oncogene homolog 1 (Abl) (bcr-abl); tyrosinase; ephrin type-A receptor 2 (EphA2); Fucosyl GM1; sialyl Lewis adhesion molecule (sLe); ganglioside GM3 (aNeu5Ac(2-3)bDGalp(1-4)bDGlcp(1-1)Cer); transglutaminase 5 (TGS5); high molecular weight-melanoma-associated antigen (HMWMAA); o-acetyl-GD2 ganglioside (OAcGD2); Folate receptor beta; tumor endothelial marker 1 (TEM1/CD248); tumor endothelial marker 7-related (TEM7R); claudin 6 (CLDN6); thyroid stimulating hormone receptor (TSHR); G protein-coupled receptor class C group 5, member D (GPRC5D); chromosome X open reading frame 61 (CXORF61); CD97; CD179a; anaplastic lymphoma kinase (ALK); Polysialic acid; placenta-specific 1 (PLAC1); hexasaccharide portion of globoH glycoceramide (GloboH); mammary gland differentiation antigen (NY-BR-1); uroplakin 2 (UPK2); Hepatitis A virus cellular receptor 1 (HAVCR1); adrenoceptor beta 3 (ADRB3); pannexin 3 (PANX3); G protein-coupled receptor 20 (GPR20); lymphocyte antigen 6 complex, locus K 9 (LY6K); Olfactory receptor 51E2 (OR51E2); TCR Gamma Alternate Reading Frame Protein (TARP); Wilms tumor protein (WT1); Cancer/testis antigen 1 (NY-ESO-1); Cancer/testis antigen 2 (LAGE-la); Melanoma-associated antigen 1 (MAGE-A1); ETS translocation-variant gene 6, located on chromosome 12p (ETV6-AML); sperm protein 17 (SPA17); X Antigen Family, Member 1A (XAGE1); angiopoietin-binding cell surface receptor 2 (Tie 2); melanoma cancer testis antigen-1 (MAD-CT-1); melanoma cancer testis antigen-2 (MAD-CT-2); Fos-related antigen 1; tumor protein p53 (p53); p53 mutant; prostein; surviving; telomerase; prostate carcinoma tumor antigen-1 (PCTA-1 or Galectin 8), melanoma antigen recognized by T cells 1 (MelanA or MART1); Rat sarcoma (Ras) mutant; human Telomerase reverse transcriptase (hTERT); sarcoma translocation breakpoints; melanoma inhibitor of apoptosis (ML-IAP); ERG (transmembrane protease, serine 2 (TMPRSS2) ETS fusion gene); N-Acetyl glucosaminyl-transferase V (NA17); paired box protein Pax-3 (PAX3); Androgen receptor; Cyclin B1; v-myc avian myelocytomatosis viral oncogene neuroblastoma derived homolog (MYCN); Ras Homolog Family Member C (RhoC); Tyrosinase-related protein 2 (TRP-2); Cytochrome P450 1B1 (CYP1B1); CCCTC-Binding Factor (Zinc Finger Protein)-Like (BORIS or Brother of the Regulator of Imprinted Sites), Squamous Cell Carcinoma Antigen Recognized By T Cells 3 (SART3); Paired box protein Pax-5 (PAX5); proacrosin binding protein sp32 (OY-TES1); lymphocyte-specific protein tyrosine kinase (LCK); A kinase anchor protein 4 (AKAP-4); synovial sarcoma, X breakpoint 2 (SSX2); Receptor for Advanced Glycation Endproducts (RAGE-1); renal ubiquitous 1 (RU1); renal ubiquitous 2 (RU2); legumain; human papilloma virus E6 (HPV E6); human papilloma virus E7 (HPV E7); intestinal carboxyl esterase; heat shock protein 70-2 mutated (mut hsp70-2); CD79a; CD79b; CD72; Leukocyte-associated immunoglobulin-like receptor 1 (LAIR1); Fc fragment of IgA receptor (FCAR or CD89); Leukocyte immunoglobulin-like receptor subfamily A member 2 (LILRA2); CD300 molecule-like family member f (CD300LF); C-type lectin domain family 12 member A (CLEC12A); bone marrow stromal cell antigen 2 (BST2); EGF-like module-containing mucin-like hormone receptor-like 2 (EMR2); lymphocyte antigen 75 (LY75); Glypican-3 (GPC3); Fc receptor-like 5 (FCRL5); and immunoglobulin lambda-like polypeptide 1 (IGLL1).

In some embodiments, the antigen binding domain comprises one, two three (for example, all three) heavy chain CDRs, HC CDR1, HC CDR2 and HC CDR3, from an antibody listed above, and/or one, two, three (for example, all three) light chain CDRs, LC CDR1, LC CDR2 and LC CDR3, from an antibody listed above. In some embodiments, the antigen binding domain comprises a heavy chain variable region and/or a variable light chain region of an antibody listed or described above.

In some embodiments, the anti-tumor antigen binding domain is a fragment, for example, a single chain variable fragment (scFv). In some embodiments, the anti-a cancer associate antigen as described herein binding domain is a Fv, a Fab, a (Fab′)2, or a bi-functional (for example bi-specific) hybrid antibody (for example, Lanzavecchia et al., Eur. J. Immunol. 17, 105 (1987)). In some embodiments, the antibodies and fragments thereof of the invention binds a cancer associate antigen as described herein protein with wild-type or enhanced affinity.

In some instances, scFvs can be prepared according to a method known in the art (see, for example, Bird et al., (1988) Science 242:423-426 and Huston et al., (1988) Proc. Natl. Acad. Sci. USA 85:5879-5883). ScFv molecules can be produced by linking VH and VL regions together using flexible polypeptide linkers. The scFv molecules comprise a linker (for example, a Ser-Gly linker) with an optimized length and/or amino acid composition. The linker length can greatly affect how the variable regions of a scFv fold and interact. In fact, if a short polypeptide linker is employed (for example, between 5-10 amino acids) intrachain folding is prevented. Interchain folding is also required to bring the two variable regions together to form a functional epitope binding site. For examples of linker orientation and size see, for example, Hollinger et al. 1993 Proc Natl Acad. Sci. U.S.A. 90:6444-6448, U.S. Patent Application Publication Nos. 2005/0100543, 2005/0175606, 2007/0014794, and PCT publication Nos. WO2006/020258 and WO2007/024715, which are incorporated herein by reference.

An scFv can comprise a linker of at least 1, 2, 3, 4, 5, 6, 7, 8, 9, 10, 11, 12, 13, 14, 15, 16, 17, 18, 19, 20, 25, 30, 35, 40, 45, 50, or more amino acid residues between its VL and VH regions. The linker sequence may comprise any naturally occurring amino acid. In some embodiments, the linker sequence comprises amino acids glycine and serine. In some embodiments, the linker sequence comprises sets of glycine and serine repeats such as (Gly₄Ser)n, where n is a positive integer equal to or greater than 1 (SEQ ID NO: 25). In some embodiments, the linker can be (Gly₄Ser)₄ (SEQ ID NO: 27) or (Gly₄Ser)₃(SEQ ID NO: 28). Variation in the linker length may retain or enhance activity, giving rise to superior efficacy in activity studies.

In some embodiments, the antigen binding domain is a T cell receptor (“TCR”), or a fragment thereof, for example, a single chain TCR (scTCR). Methods to make such TCRs are known in the art. See, for example, Willemsen R A et al, Gene Therapy 7: 1369-1377 (2000); Zhang T et al, Cancer Gene Ther 11: 487-496 (2004); Aggen et al, Gene Ther. 19(4):365-74 (2012) (references are incorporated herein by its entirety). For example, scTCR can be engineered that contains the Vα and Vβ genes from a T cell clone linked by a linker (for example, a flexible peptide). This approach is very useful to cancer associated target that itself is intracellular, however, a fragment of such antigen (peptide) is presented on the surface of the cancer cells by MHC.

### Transmembrane Domain

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, for example, one or more amino acid associated with the extracellular region of the protein from which the transmembrane was derived (for example, 1, 2, 3, 4, 5, 6, 7, 8, 9, 10 up to 15 amino acids of the extracellular region) and/or one or more additional amino acids associated with the intracellular region of the protein from which the transmembrane protein is derived (for example, 1, 2, 3, 4, 5, 6, 7, 8, 9, 10 up to 15 amino acids of the intracellular region). In some embodiments, the transmembrane domain is one that is associated with one of the other domains of the CAR is used. In some instances, the transmembrane domain can be selected or modified by amino acid substitution to avoid binding of such domains to the transmembrane domains of the same or different surface membrane proteins, for example, to minimize interactions with other members of the receptor complex. In some embodiments, the transmembrane domain is capable of homodimerization with another CAR on the CAR-expressing cell, for example, CART cell, surface. In some embodiments the amino acid sequence of the transmembrane domain may be modified or substituted so as to minimize interactions with the binding domains of the native binding partner present in the same CAR-expressing cell, for example, CART.

The transmembrane domain may be derived either from a natural or from a recombinant source. Where the source is natural, the domain may be derived from any membrane-bound or transmembrane protein. In some embodiments the transmembrane domain is capable of signaling to the intracellular domain(s) whenever the CAR has bound to a target. A transmembrane domain of particular use in this invention may include at least the transmembrane region(s) of, for example, the alpha, beta or zeta chain of T-cell receptor, CD28, CD3 epsilon, CD45, CD4, CD5, CD8 (for example, CD8 alpha, CD8 beta), CD9, CD16, CD22, CD33, CD37, CD64, CD80, CD86, CD134, CD137, CD154. In some embodiments, a transmembrane domain may include at least the transmembrane region(s) of a costimulatory molecule, for example, MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In some instances, the transmembrane domain can be attached to the extracellular region of the CAR, for example, the antigen binding domain of the CAR, via a hinge, for example, a hinge from a human protein. For example, in some embodiments, the hinge can be a human Ig (immunoglobulin) hinge, for example, an IgG4 hinge, or a CD8a hinge. In some embodiments, the hinge or spacer comprises (for example, consists of) the amino acid sequence of SEQ ID NO: 2. In some embodiments, the transmembrane domain comprises (for example, consists of) a transmembrane domain of SEQ ID NO: 6.

In some embodiments, the hinge or spacer comprises an IgG4 hinge. For example, in some embodiments, the hinge or spacer comprises a hinge of SEQ ID NO: 3. In some embodiments, the hinge or spacer comprises a hinge encoded by the nucleotide sequence of SEQ ID NO: 14.

In some embodiments, the hinge or spacer comprises an IgD hinge. For example, in some embodiments, the hinge or spacer comprises a hinge of the amino acid sequence of SEQ ID NO: 4. In some embodiments, the hinge or spacer comprises a hinge encoded by the nucleotide sequence of SEQ ID NO:15.

In some embodiments, the transmembrane domain may be recombinant, in which case it will comprise predominantly hydrophobic residues such as leucine and valine. In some embodiments, a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in some embodiments, the linker comprises the amino acid sequence of SEQ ID NO: 5. In some embodiments, the linker is encoded by a nucleotide sequence of SEQ ID NO: 16.

In some embodiments, the hinge or spacer comprises a KIR2DS2 hinge.

### Cytoplasmic Domain

The cytoplasmic domain or region of a CAR of the present invention includes an intracellular signaling domain. An intracellular signaling domain is generally responsible for activation of at least one of the normal effector functions of the immune cell in which the CAR has been introduced.

Examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, for example, a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the invention include those of TCR zeta, FcR gamma, FcR beta, CD3 gamma, CD3 delta, CD3 epsilon, CD5, CD22, CD79a, CD79b, CD278 (also known as “ICOS”), FcεRI, DAP10, DAP12, and CD66d. In some embodiments, a CAR of the invention comprises an intracellular signaling domain, for example, a primary signaling domain of CD3-zeta.

In some embodiments, a primary signaling domain comprises a modified ITAM domain, for example, a mutated ITAM domain which has altered (for example, increased or decreased) activity as compared to the native ITAM domain. In some embodiments, a primary signaling domain comprises a modified ITAM-containing primary intracellular signaling domain, for example, an optimized and/or truncated ITAM-containing primary intracellular signaling domain. In some embodiments, a primary signaling domain comprises one, two, three, four or more ITAM motifs.

Further examples of molecules containing a primary intracellular signaling domain that are of particular use in the invention include those of DAP10, DAP12, and CD32.

The intracellular signaling domain of the CAR can comprise the primary signaling domain, for example, CD3-zeta signaling domain, by itself or it can be combined with any other desired intracellular signaling domain(s) useful in the context of a CAR of the invention. For example, the intracellular signaling domain of the CAR can comprise a primary signaling domain, for example, CD3 zeta chain portion, and a costimulatory signaling domain. The costimulatory signaling domain refers to a portion of the CAR comprising the intracellular domain of a costimulatory molecule. A costimulatory molecule is a cell surface molecule other than an antigen receptor or its ligands that is required for an efficient response of lymphocytes to an antigen. Examples of such molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). The intracellular signaling sequences within the cytoplasmic portion of the CAR of the invention may be linked to each other in a random or specified order. Optionally, a short oligo- or polypeptide linker, for example, between 2 and 10 amino acids (for example, 2, 3, 4, 5, 6, 7, 8, 9, or 10 amino acids) in length may form the linkage between intracellular signaling sequence. In some embodiments, a glycine-serine doublet can be used as a suitable linker. In some embodiments, a single amino acid, for example, an alanine, a glycine, can be used as a suitable linker.

In some embodiments, the intracellular signaling domain is designed to comprise two or more, for example, 2, 3, 4, 5, or more, costimulatory signaling domains. In some embodiments, the two or more, for example, 2, 3, 4, 5, or more, costimulatory signaling domains, are separated by a linker molecule, for example, a linker molecule described herein. In some embodiments, the intracellular signaling domain comprises two costimulatory signaling domains. In some embodiments, the linker molecule is a glycine residue. In some embodiments, the linker is an alanine residue.

In some embodiments, the intracellular signaling domain is designed to comprise the signaling domain of CD3-zeta and the signaling domain of CD28. In some embodiments, the intracellular signaling domain is designed to comprise the signaling domain of CD3-zeta and the signaling domain of 4-1BB. In some embodiments, the signaling domain of 4-1BB is a signaling domain of SEQ ID NO: 7. In some embodiments, the signaling domain of CD3-zeta is a signaling domain of SEQ ID NO: 9 (mutant CD3zeta) or SEQ ID NO: 10 (wild type human CD3zeta).

In some embodiments, the intracellular signaling domain is designed to comprise the signaling domain of CD3-zeta and the signaling domain of CD27. In some embodiments, the signaling domain of CD27 comprises the amino acid sequence of SEQ ID NO: 8. In some embodiments, the signaling domain of CD27 is encoded by the nucleic acid sequence of SEQ ID NO: 19.

In some embodiments, the intracellular is designed to comprise the signaling domain of CD3-zeta and the signaling domain of CD28. In some embodiments, the signaling domain of CD28 comprises the amino acid sequence of SEQ ID NO: 36. In some embodiments, the signaling domain of CD28 is encoded by the nucleic acid sequence of SEQ ID NO: 37.

In some embodiments, the intracellular is designed to comprise the signaling domain of CD3-zeta and the signaling domain of ICOS. In some embodiments, the signaling domain of ICOS comprises the amino acid sequence of SEQ ID NO: 38. In some embodiments, the signaling domain of ICOS is encoded by the nucleic acid sequence of SEQ ID NO: 39.

### CAR Configurations

### Dual CARs

In an embodiment, an immune cell (e.g., a T cell or NK cell) expresses two CARs, e.g., a first CAR that binds to a first antigen and a second CAR that binds to a second antigen. In an embodiment, the first antigen and the second antigen are different. In an embodiment, the first or second antigen is chosen from an antigen expressed on B cells, an antigen expressed on acute myeloid leukemia cells, or an antigen on solid tumor cells. In an embodiment, the first or second antigen is chosen from CD10, CD19, CD20, CD22, CD34, CD123, BCMA, FLT-3, ROR1, CD79b, CD179b, CD79a, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

In an embodiment, the first antigen is CD19. In an embodiment, the second antigen is not CD19. In an embodiment, the second antigen is an antigen disclosed herein that is not CD19.

In an embodiment, the first antigen is BCMA. In an embodiment, the second antigen is not BCMA. In an embodiment, the second antigen is an antigen disclosed herein that is not BCMA. In an embodiment, the second antigen is chosen from an antigen expressed on B cells, an antigen expressed on acute myeloid leukemia cells, or an antigen on solid tumor cells. In an embodiment, the second antigen is chosen from CD10, CD19, CD20, CD22, CD34, CD123, FLT-3, ROR1, CD79b, CD179b, CD79a, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC. In an embodiment, the first antigen is BCMA and the second antigen is CD19.

In an embodiment, the first CAR is encoded by a first nucleic acid sequence. In an embodiment, the second CAR is encoded by a second nucleic acid sequence. In an embodiment, the first and second nucleic acid sequences are disposed on a single nucleic acid molecule. In an embodiment, the first and second nucleic acid sequences are disposed on separate nucleic acid molecules. In an embodiment, the nucleic acid molecule or nucleic acid molecules are DNA or RNA molecules. In embodiments, the first and second nucleic acid sequences are situated in the same orientation, e.g., transcription of the first and second nucleic acid sequences proceeds in the same direction. In embodiments, the first and second nucleic acid sequences are situated in different orientations. In embodiments, a single promoter controls expression of the first and second nucleic acid sequences. In embodiments, a nucleic acid encoding a protease cleavage site (such as a T2A, P2A, E2A, or F2A cleavage site) is situated between the first and second nucleic acid sequences. In embodiments, the protease cleavage site is placed such that a cell can express a fusion protein comprising the first CAR and the second CAR and the fusion protein is subsequently processed into two peptides by proteolytic cleavage. In some embodiments, the first nucleic acid sequence is upstream of the second nucleic acid sequence, or the second nucleic acid sequence is upstream of the first nucleic acid sequence. In embodiments, a first promoter controls expression of the first nucleic acid sequence and a second promoter controls expression of the second nucleic acid sequence. In embodiments, the nucleic acid molecule is a plasmid. In embodiments, the nucleic acid molecule comprises a viral packaging element. In embodiments, the immune cell may comprise a protease (e.g., endogenous or exogenous protease) that cleaves a T2A, P2A, E2A, or F2A cleavage site.

In an embodiment, the first CAR comprises a first antigen-binding domain and the second CAR comprises a second antigen-binding domain. In an embodiment, the first or second antigen binding domain comprises a CDR, a VH, a VL, or a scFv disclosed herein, or an amino acid sequence having at least about 85%, 90%, 95%, or 99% sequence identity thereto. In an embodiment, the first or second antigen binding domain comprises a CDR, a VH, a VL, or a scFv of an anti-BCMA antigen binding domain disclosed herein (e.g., an amino acid sequence disclosed in Tables 3-15, 19, 20, 22, and 26, or an amino acid sequence having at least about 85%, 90%, 95%, or 99% sequence identity thereto). In an embodiment, the first or second antigen binding domain comprises a CDR, a VH, a VL, or a scFv of an anti-CD19 antigen binding domain disclosed herein (e.g., an amino acid sequence disclosed in Tables 2, 19, and 22, or an amino acid sequence having at least about 85%, 90%, 95%, or 99% sequence identity thereto).

In an embodiment, the first antigen is BCMA and the second antigen is CD19. In an embodiment, an immune cell (e.g., a T cell or NK cell) expresses an anti-BCMA CAR, e.g., an anti-BCMA CAR described herein and an anti-CD19 CAR, e.g., an anti-CD19 CAR described herein. In an embodiment, the immune cell (e.g., a T cell or NK cell) comprises a first nucleic acid sequence encoding an anti-BCMA CAR, e.g., an anti-BCMA CAR described herein and a second nucleic acid sequence encoding an anti-CD19 CAR, e.g., an anti-CD19 CAR described herein. Table 19 shows exemplary amino acid and nucleic acid sequences of dual CAR constructs.

TABLE 19
Exemplary BCMA/CD19 dual CAR constructs and components thereof
Dual BCMA/CD19 constructs

| Identification | Protein sequence | DNA sequence (5′-3′) |
|---|---|---|
| Signal peptide | MALPVTALLLPLALLLHAARP | Atggccctccctgtcaccgccctgctgcttccgct |
| (in the anti- | (SEQ ID NO: 1) | ggctcttctgctccacgccgctcggccc (SEQ |
| BCMA CAR arm) | | ID NO: 199) |
| ScFv R1G5 | EVQLLESGGGLVQPGGSLRLSCAASG | Gaagtgcagttgctggagtcaggcggaggactggt |
| | FTFSSYAMSWVRQAPGKGLEWVSAIS | gcagcccggaggatcgcttcgcttgagctgcgcag |
| | GSGGSTYYADSVKGRFTISRDNSKNT | cctcaggctttaccttctcctcctacgccatgtcc |
| | LYLQMNSLRAEDTAVYYCARREWWGE | tgggtcagacaggctcccgggaagggactggaatg |
| | SWLFDYWGQGTLVTVSSGGGGSGGGG | ggtgtccgccattagcggttccggcggaagcactt |
| | SGGGGSGGGGSDIQMTQSPSSLSASV | actatgccgactctgtgaagggccgcttcactatc |
| | GDRVTITCRASQSISSYLNWYQQKPG | tcccgggacaactccaagaacaccctgtatctcca |
| | KAPKLLIYAASSLQSGVPSRFSGSGS | aatgaattccctgagggccgaagataccgcggtgt |
| | GTDFTLTISSLQPEDFATYYCQQSYS | actactgcgctagacgggagtggtggggagaaagc |
| | TPLTFGQGTKVEIK (SEQ ID | tggctgttcgactactggggacagggcactctcgt |
| | NO: 80) | gactgtgtcctccggtggtggtggatcggggggtg |
| | | gtggttcgggcggaggaggatctggaggaggaggg |
| | | tcggacattcaaatgactcagtccccgtcctccct |
| | | ctccgcctccgtgggagatcgcgtcacgatcacgt |
| | | gcagggccagccagagcatctccagctacctgaac |
| | | tggtaccagcagaagccagggaaggcaccgaagct |
| | | cctgatctacgccgctagctcgctgcagtccggcg |
| | | tcccttcacggttctcgggatcgggctcaggcacc |
| | | gacttcaccctgaccattagcagcctgcagccgga |
| | | ggacttcgcgacatactactgtcagcagtcatact |
| | | ccacccctctgaccttcggccaagggaccaaagtg |
| | | gagatcaag (SEQ ID NO: 81) |
| ScFv PI61 | QVQLQESGGGVVQPGRSLRLSCAASG | Caagtgcagctgcaggaatccggtggcggagtcgt |
| | FTFSSYGMHWVRQAPGKGLEWVAVIS | gcagcctggaaggagcctgagactctcatgcgccg |
| | YDGSNKYYADSVKGRFTISRDNSKNT | cgtcagggttcaccttttcctcctacgggatgcat |
| | LYLQMNSLRAEDTAVYYCGGSGYALH | tgggtcagacaggcccccggaaagggactcgaatg |
| | DDYYGLDVWGQGTLVTVSSGGGGSGG | ggtggctgtgatcagctacgacggctccaacaagt |
| | GGSGGGGSQSALTQPASVSGSPGQSI | actacgccgactccgtgaaaggccggttcactatc |
| | TISCTGTSSDVGGYNYVSWYQQHPGK | tcccgggacaactccaagaacacgctgtatctgca |
| | APKLMIYDVSNRPSGVSNRFSGSKSG | aatgaattcactgcgcgcggaggataccgctgtgt |
| | NTASLTISGLQAEDEADYYCSSYTSS | actactgcggtggctccggttacgccctgcacgat |
| | STLYVFGSGTKVTVL (SEQ ID | gactattacggccttgacgtctggggccagggaac |
| | NO: 105) | cctcgtgactgtgtccagcggtggaggaggttcgg |
| | | gcggaggaggatcaggagggggtggatcgcagagc |
| | | gcactgactcagccggcatccgtgtccggtagccc |
| | | cggacagtcgattaccatctcctgtaccggcacct |
| | | cctccgacgtggggggtacaactacgtgtcgtgg |
| | | taccagcagcacccaggaaaggcccctaagttgat |
| | | gatctacgatgtgtcaaaccgcccgtctggagtct |
| | | ccaaccggttctccggctccaagtccggcaacacc |
| | | gccagcctgaccattaggggctgcaagccgagga |
| | | tgaggccgactactactgctcgagctacacatcct |
| | | cgagcaccctctacgtgttcggctcggggactaag |
| | | gtcaccgtgctg (SEQ ID NO: 106) |
| ScFv R1B6 | EVQLLESGGGLVQPGGSLRLSCAASG | Gaagtgcagttgctggagtcaggcggaggactggt |
| | FTFSSYAMSWVRQAPGKGLEWVSAIS | gcagcccggaggatcgcttcgcttgagctgcgcag |
| | GSGGSTYYADSVKGRFTISRDNSKNT | cctcaggctttaccttctcctcctacgccatgtcc |
| | LYLQMNSLRAEDTAVYYCARREWVPY | tgggtcagacaggctcccgggaagggactggaatg |
| | DVSWYFDYWGQGTLVTVSSGGGGSGG | ggtgtccgccattagcggttccggcggaagcactt |
| | GGSGGGGSGGGGSDIQMTQSPSSLSA | actatgccgactctgtgaagggccgcttcactatc |
| | SVGDRVTITCRASQSISSYLNWYQQK | tcccgggacaactccaagaacaccctgtatctcca |
| | PGKAPKLLIYAASSLQSGVPSRFSGS | aatgaattccctgagggccgaagataccgcggtgt |
| | GSGTDFTLTISSLQPEDFATYYCQQS | actactgcgctagacgggagtgggtgccctacgat |
| | YSTPLTFGQGTKVEIK (SEQ ID | gtcagctggtacttcgactactggggacagggcac |
| | NO: 64) | tctcgtgactgtgtcctccggtggtggtggatcgg |
| | | ggggtggtggttcgggcggaggaggatctggagga |
| | | ggagggtcggacattcaaatgactcagtccccgtc |
| | | ctccctctccgcctccgtgggagatcgcgtcacga |
| | | tcacgtgcagggccagccagagcatctccagctac |
| | | ctgaactggtaccagcagaagccagggaaggcacc |
| | | gaagctcctgatctacgccgctagctcgctgcagt |
| | | ccggcgtcccttcacggttctcgggatcgggctca |
| | | ggcaccgacttcaccctgaccattagcagcctgca |
| | | gccggaggacttcgcgacatactactgtcagcagt |
| | | catactccacccctctgaccttcggccaagggacc |
| | | aaagtggagatcaag (SEQ ID NO: 65) |
| ScFv | EVQLVESGGGLVQPGGSLRLSCAVSG | Gaagtgcaattggtggaatcagggggaggacttgt |
| duBCMA.4 | FALSNHGMSWVRRAPGKGLEWVSGIV | gcagcctggaggatcgctgagactgtcatgtgccg |
| | YSGSTYYAASVKGRFTISRDNSRNTL | tgtccggctttgccctgtccaaccacgggatgtcc |
| | YLQMNSLRPEDTAIYYCSAHGGESDV | tgggtccgccgcgcgcctggaaagggcctcgaatg |
| | WGQGTTVTVSSASGGGGSGGGGSGGG | ggtgtcgggtattgtgtacagcggtagcacctact |
| | GSDIQLTQSPSSLSASVGDRVTITCR | atgccgcatccgtgaaggggagattcaccatcagc |
| | ASQSISSYLNWYQQKPGKAPKLLIYA | cgggacaactccaggaacactctgtacctccaaat |
| | ASSLQSGVPSRFSGSGSGTDFTLTIS | gaattcgctgaggccagaggacactgccatctact |
| | SLQPEDFATYYCQQSYSTPYTFGQGT | actgctccgcgcatggcggagagtccgacgtctgg |
| | KVEIK (SEQ ID NO: 200) | ggacaggggaccaccgtgaccgtgtctagcgcgtc |
| | | cggcggaggcggcagcgggggtggtggttcagggg |
| | | gcggcggatcggacatccagctcacccagtccccg |
| | | agctcgctgtccgcctccgtgggagatcgggtcac |
| | | catcacgtgccgcgccagccagtcgatttcctcct |
| | | acctgaactggtaccaacagaagcccggaaaagcc |
| | | ccgaagcttctcatctacgccgcctcgagcctgca |
| | | gtcaggagtgccctcacggttctccggctccggtt |
| | | ccggtactgatttcaccctgaccatttcctccctg |
| | | caaccggaggacttcgctacttactactgccagca |
| | | gtcgtactccaccccctacactttcggacaaggca |
| | | ccaaggtcgaaatcaag (SEQ ID NO: 201) |
| Hinge and | TTTPAPRPPTPAPTIASQPLSLRPEA | Accactaccccagcaccgaggccacccaccccggc |
| transmembrane | CRPAAGGAVHTRGLDFACDIYIWAPL | tcctaccatcgcctcccagcctctgtccctgcgtc |
| domain (in | AGTCGVLLLSLVITLYC (SEQ ID | cggaggcatgtagacccgcagctggtggggccgtg |
| the anti-BCMA | NO: 202) | catacccggggtcttgacttcgcctgcgatatcta |
| CAR arm) | | catttgggcccctctggctggtacttgcggggtcc |
| | | tgctgctttcactcgtgatcactctttactgt |
| | | (SEQ ID NO: 203) |
| Hinge (in the | TTTPAPRPPTPAPTIASQPLSLRPEA | Accactaccccagcaccgaggccacccaccccggc |
| anti-BCMA | CRPAAGGAVHTRGLDFACD (SEQ | tcctaccatcgcctcccagcctctgtccctgcgtc |
| CAR arm) | ID NO: 2) | cggaggcatgtagacccgcagctggtggggccgtg |
| | | catacccggggtcttgacttcgcctgcgat (SEQ |
| | | ID NO: 337) |
| Transmembrane | IYIWAPLAGTCGVLLLSLVITLYC | Atctacatttgggcccctctggctggtacttgcgg |
| domain (in the | (SEQ ID NO: 6) | ggtcctgctgctttcactcgtgatcactctttact |
| anti-BCMA CAR | | gt (SEQ ID NO: 338) |
| arm) | | |
| 4-1BB (in the | KRGRKKLLYIFKQPFMRPVQTTQEED | Aagcgcggtcggaagaagctgctgtacatctttaa |
| anti-BCMA CAR | GCSCRFPEEEEGGCEL (SEQ ID | gcaacccttcatgaggcctgtgcagactactcaag |
| arm) | NO: 7) | aggaggacggctgttcatgccggttcccagaggag |
| | | gaggaaggcggctgcgaactg (SEQ ID NO: |
| | | 204) |
| CD3zeta (in | RVKFSRSADAPAYQQGQNQLYNELNL | Cgcgtgaaattcagccgcagcgcagatgctccagc |
| the anti-BCMA | GRREEYDVLDKRRGRDPEMGGKPRRK | ctaccagcaggggcagaaccagctctacaacgaac |
| CAR arm) | NPQEGLYNELQKDKMAEAYSEIGMKG | tcaatcttggtcggagagaggagtacgacgtgctg |
| | ERRRGKGHDGLYQGLSTATKDTYDAL | gacaagcggagaggacgggacccagaaatgggcgg |
| | HMQALPPR (SEQ ID NO: 10) | gaagccgcgcagaaagaatccccaagagggcctgt |
| | | acaacgagctccaaaaggataagatggcagaagcc |
| | | tatagcgagattggtatgaaaggggaacgcagaag |
| | | aggcaaaggccacgacggactgtaccagggactca |
| | | gcaccgccaccaaggacacctatgacgctcttcac |
| | | atgcaggccctgccgcctcgg (SEQ ID NO: |
| | | 205) |
| Linker | GSG (SEQ ID NO: 206) | Ggaagcgga (SEQ ID NO: 207) |
| P2A | ATNFSLLKQAGDVEENPGP (SEQ | Gctactaacttcagcctgctgaagcaggctggaga |
| sequence | ID NO: 208) | cgtggaggagaaccctggacct (SEQ ID NO: |
| | | 209) |
| Signal peptide | MALPVTALLLPLALLLHAARP (SEQ | Atggccttaccagtgaccgccttgctcctgccgct |
| (in the anti- | ID NO: 1) | ggccttgctgctccacgccgccaggccg (SEQ |
| CD19 CAR arm) | | ID NO: 210) |
| ScFv | EIVMTQSPATLSLSPGERATLSCRAS | Gaaattgtgatgacccagtcacccgccactcttag |
| duCD19.1 | QDISKYLNWYQQKPGQAPRLLIYHTS | cctttcacccggtgagcgcgcaaccctgtcttgca |
| | RLHSGIPARFSGSGSGTDYTLTISSL | gagcctcccaagacatctcaaaataccttaattgg |
| | QPEDFAVYFCQQGNTLPYTFGQGTKL | tatcaacagaagcccggacaggctcctcgccttct |
| | EIKGGGGSGGGGSGGGGSQVQLQESG | gatctaccacaccagccggctccattctggaatcc |
| | PGLVKPSETLSLTCTVSGVSLPDYGV | ctgccaggttcagcggtagcggatctgggaccgac |
| | SWIRQPPGKGLEWIGVIWGSETTYYQ | tacaccctcactatcagctcactgcagccagagga |
| | SSLKSRVTISKDNSKNQVSLKLSSVT | cttcgctgtctatttctgtcagcaagggaacaccc |
| | AADTAVYYCAKHYYYGGSYAMDYWGQ | tgccctacacctttggacagggcaccaagctcgag |
| | GTLVTVSS (SEQ ID NO: 211) | attaaaggtggaggtggcagcggaggaggtgggtc |
| | | cggcggtggaggaagccaggtccaactccaagaaa |
| | | gcggaccgggtcttgtgaagccatcagaaactctt |
| | | tcactgacttgtactgtgagcggagtgtctctccc |
| | | cgattacggggtgtcttggatcagacagccaccgg |
| | | ggaagggtctggaatggattggagtgatttggggc |
| | | tctgagactacttactaccaatcatccctcaagtc |
| | | acgcgtcaccatctcaaaggacaactctaagaatc |
| | | aggtgtcactgaaactgtcatctgtgaccgcagcc |
| | | gacaccgccgtgtactattgcgctaagcattacta |
| | | ttatggcgggagctacgcaatggattactggggac |
| | | agggtactctggtcaccgtgtccagc (SEQ ID |
| | | NO: 212) |
| Hinge and | TTTPAPRPPTPAPTIASQPLSLRPEA | Accacgacgccagcgccgcgaccaccaacaccggc |
| transmembrane | CRPAAGGAVHTRGLDFACDIYIWAPL | gcccaccatcgcgtcgcagcccctgtccctgcgcc |
| domain (in | AGTCGVLLLSLVITLYC (SEQ ID | cagaggcgtgccggccagcggcggggggcgcagtg |
| the anti-CD19 | NO: 202) | cacacgagggggctggacttcgcctgtgatatcta |
| CAR arm) | | catctgggcgcccttggccgggacttgtggggtcc |
| | | ttctcctgtcactggttatcaccctttactgc |
| | | (SEQ ID NO: 213) |
| Hinge (in the | TTTPAPRPPTPAPTIASQPLSLRPEA | Accacgacgccagcgccgcgaccaccaacaccggc |
| anti-CD19 CAR | CRPAAGGAVHTRGLDFACD (SEQ | gcccaccatcgcgtcgcagcccctgtccctgcgcc |
| arm) | ID NO: 2) | cagaggcgtgccggccagcggcggggggcgcagtg |
| | | cacacgagggggctggacttcgcctgtgat (SEQ |
| | | ID NO: 13) |
| Transmembrane | IYIWAPLAGTCGVLLLSLVITLYC | Atctacatctgggcgcccttggccgggacttgtgg |
| domain (in | (SEQ ID NO: 6) | ggtccttctcctgtcactggttatcaccctttact |
| the anti-CD19 | | gc (SEQ ID NO: 17) |
| CAR arm) | | |
| 4-1BB (in the | KRGRKKLLYIFKQPFMRPVQTTQEED | Aaacggggcagaaagaaactcctgtatatattcaa |
| anti-CD19 CAR | GCSCRFPEEEEGGCEL (SEQ ID | acaaccatttatgagaccagtacaaactactcaag |
| arm) | NO: 7) | aggaagatggctgtagctgccgatttccagaagaa |
| | | gaagaaggaggatgtgaactg (SEQ ID NO: |
| | | 18) |
| CD3zeta (in | RVKFSRSADAPAYQQGQNQLYNELNL | Agagtgaagttcagcaggagcgcagacgcccccgc |
| the anti-CD19 | GRREEYDVLDKRRGRDPEMGGKPRRK | gtaccagcagggccagaaccagctctataacgagc |
| CAR arm) | NPQEGLYNELQKDKMAEAYSEIGMKG | tcaatctaggacgaagagaggagtacgatgttttg |
| | ERRRGKGHDGLYQGLSTATKDTYDAL | gacaagagacgtggccgggaccctgagatgggggg |
| | HMQALPPR (SEQ ID NO: 10) | aaagccgagaaggaagaaccctcaggaaggcctgt |
| | | acaatgaactgcagaaagataagatggcggaggcc |
| | | tacagtgagattgggatgaaaggcgagcgccggag |
| | | gggcaaggggcacgatggcctttaccagggtctca |
| | | gtacagccaccaaggacacctacgacgcccttcac |
| | | atgcaggccctgccccctcgc (SEQ ID NO: |
| | | 21) |
| R1G5-P2A- | MALPVTALLLPLALLLHAARPEVQLL | atggccctccctgtcaccgccctgctgcttccgct |
| duCD19.1 | ESGGGLVQPGGSLRLSCAASGFTFSS | ggctcttctgctccacgccgctcggcccgaagtgc |
| | YAMSWVRQAPGKGLEWVSAISGSGGS | agttgctggagtcaggcggaggactggtgcagccc |
| | TYYADSVKGRFTISRDNSKNTLYLQM | ggaggatcgcttcgcttgagctgcgcagcctcagg |
| | NSLRAEDTAVYYCARREWWGESWLED | ctttaccttctcctcctacgccatgtcctgggtca |
| | YWGQGTLVTVSSGGGGSGGGGSGGGG | gacaggctcccgggaagggactggaatgggtgtcc |
| | SGGGGSDIQMTQSPSSLSASVGDRVT | gccattagcggttccggcggaagcacttactatgc |
| | ITCRASQSISSYLNWYQQKPGKAPKL | cgactctgtgaagggccgcttcactatctcccggg |
| | LIYAASSLQSGVPSRFSGSGSGTDFT | acaactccaagaacaccctgtatctccaaatgaat |
| | LTISSLQPEDFATYYCQQSYSTPLTF | tccctgagggccgaagataccgcggtgtactactg |
| | GQGTKVEIKTTTPAPRPPTPAPTIAS | cgctagacgggagtggtggggagaaagctggctgt |
| | QPLSLRPEACRPAAGGAVHTRGLDFA | tcgactactggggacagggcactctcgtgactgtg |
| | CDIYIWAPLAGTCGVLLLSLVITLYC | tcctccggtggtggtggatcggggggtggtggttc |
| | KRGRKKLLYIFKQPFMRPVQTTQEED | gggcggaggaggatctggaggaggagggtcggaca |
| | GCSCRFPEEEEGGCELRVKFSRSADA | ttcaaatgactcagtccccgtcctccctctccgcc |
| | PAYQQGQNQLYNELNLGRREEYDVLD | tccgtgggagatcgcgtcacgatcacgtgcagggc |
| | KRRGRDPEMGGKPRRKNPQEGLYNEL | cagccagagcatctccagctacctgaactggtacc |
| | QKDKMAEAYSEIGMKGERRRGKGHDG | agcagaagccagggaaggcaccgaagctcctgatc |
| | LYQGLSTATKDTYDALHMQALPPRGS | tacgccgctagctcgctgcagtccggcgtcccttc |
| | GATNFSLLKQAGDVEENPGPMALPVT | acggttctcgggatcgggctcaggcaccgacttca |
| | ALLLPLALLLHAARPEIVMTQSPATL | ccctgaccattagcagcctgcagccggaggacttc |
| | SLSPGERATLSCRASQDISKYLNWYQ | gcgacatactactgtcagcagtcatactccacccc |
| | QKPGQAPRLLIYHTSRLHSGIPARFS | tctgaccttcggccaagggaccaaagtggagatca |
| | GSGSGTDYTLTISSLQPEDFAVYFCQ | agaccactaccccagcaccgaggccacccaccccg |
| | QGNTLPYTFGQGTKLEIKGGGGSGGG | gctcctaccatcgcctcccagcctctgtccctgcg |
| | GSGGGGSQVQLQESGPGLVKPSETLS | tccggaggcatgtagacccgcagctggtggggccg |
| | LTCTVSGVSLPDYGVSWIRQPPGKGL | tgcatacccggggtcttgacttcgcctgcgatatc |
| | EWIGVIWGSETTYYQSSLKSRVTISK | tacatttgggcccctctggctggtacttgcggggt |
| | DNSKNQVSLKLSSVTAADTAVYYCAK | cctgctgctttcactcgtgatcactctttactgta |
| | HYYYGGSYAMDYWGQGTLVTVSSTTT | agcgcggtcggaagaagctgctgtacatctttaag |
| | PAPRPPTPAPTIASQPLSLRPEACRP | caacccttcatgaggcctgtgcagactactcaaga |
| | AAGGAVHTRGLDFACDIYIWAPLAGT | ggaggacggctgttcatgccggttoccagaggagg |
| | CGVLLLSLVITLYCKRGRKKLLYIFK | aggaaggcggctgcgaactgcgcgtgaaattcagc |
| | QPFMRPVQTTQEEDGCSCRFPEEEEG | cgcagcgcagatgctccagcctaccagcaggggca |
| | GCELRVKFSRSADAPAYQQGQNQLYN | gaaccagctctacaacgaactcaatcttggtcgga |
| | ELNLGRREEYDVLDKRRGRDPEMGGK | gagaggagtacgacgtgctggacaagcggagagga |
| | PRRKNPQEGLYNELQKDKMAEAYSEI | cgggacccagaaatgggcgggaagccgcgcagaaa |
| | GMKGERRRGKGHDGLYQGLSTATKDT | gaatccccaagagggcctgtacaacgagctccaaa |
| | YDALHMQALPPR (SEQ ID NO: | aggataagatggcagaagcctatagcgagattggt |
| | 214) | atgaaaggggaacgcagaagaggcaaaggccacga |
| | | cggactgtaccagggactcagcaccgccaccaagg |
| | | acacctatgacgctcttcacatgcaggccctgccg |
| | | cctcggggaagcggagctactaacttcagcctgct |
| | | gaagcaggctggagacgtggaggagaaccctggac |
| | | ctatggccttaccagtgaccgccttgctcctgccg |
| | | ctggccttgctgctccacgccgccaggccggaaat |
| | | tgtgatgacccagtcacccgccactcttagccttt |
| | | cacccggtgagcgcgcaaccctgtcttgcagagcc |
| | | tcccaagacatctcaaaataccttaattggtatca |
| | | acagaagcccggacaggctcctcgccttctgatct |
| | | accacaccagccggctccattctggaatccctgcc |
| | | aggttcagcggtagcggatctgggaccgactacac |
| | | cctcactatcagctcactgcagccagaggacttcg |
| | | ctgtctatttctgtcagcaagggaacaccctgccc |
| | | tacacctttggacagggcaccaagctcgagattaa |
| | | aggtggaggtggcagcggaggaggtgggtccggcg |
| | | gtggaggaagccaggtccaactccaagaaagcgga |
| | | ccgggtcttgtgaagccatcagaaactctttcact |
| | | gacttgtactgtgagcggagtgtctctccccgatt |
| | | acggggtgtcttggatcagacagccaccggggaag |
| | | ggtctggaatggattggagtgatttggggctctga |
| | | gactacttactaccaatcatccctcaagtcacgcg |
| | | tcaccatctcaaaggacaactctaagaatcaggtg |
| | | tcactgaaactgtcatctgtgaccgcagccgacac |
| | | cgccgtgtactattgcgctaagcattactattatg |
| | | gcgggagctacgcaatggattactggggacagggt |
| | | actctggtcaccgtgtccagcaccacgacgccagc |
| | | gccgcgaccaccaacaccggcgcccaccatcgcgt |
| | | cgcagcccctgtccctgcgcccagaggcgtgccgg |
| | | ccagcggggggggcgcagtgcacacgagggggct |
| | | ggacttcgcctgtgatatctacatctgggcgccct |
| | | tggccgggacttgtggggtccttctcctgtcactg |
| | | gttatcaccctttactgcaaacggggcagaaagaa |
| | | actcctgtatatattcaaacaaccatttatgagac |
| | | cagtacaaactactcaagaggaagatggctgtagc |
| | | tgccgatttccagaagaagaagaaggaggatgtga |
| | | actgagagtgaagttcagcaggagcgcagacgccc |
| | | ccgcgtaccagcagggccagaaccagctctataac |
| | | gagctcaatctaggacgaagagaggagtacgatgt |
| | | tttggacaagagacgtggccgggaccctgagatgg |
| | | ggggaaagccgagaaggaagaaccctcaggaaggc |
| | | ctgtacaatgaactgcagaaagataagatggcgga |
| | | ggcctacagtgagattgggatgaaaggcgagcgcc |
| | | ggaggggcaaggggcacgatggcctttaccagggt |
| | | ctcagtacagccaccaaggacacctacgacgccct |
| | | tcacatgcaggccctgccccctcgc (SEQ ID |
| | | NO: 215) |
| PI61-P2A- | MALPVTALLLPLALLLHAARPQVQLQ | atggccctccctgtcaccgccctgctgcttccgct |
| duCD19.1 | ESGGGVVQPGRSLRLSCAASGFTFSS | ggctcttctgctccacgccgctcggccccaagtgc |
| | YGMHWVRQAPGKGLEWVAVISYDGSN | agctgcaggaatccggtggcggagtcgtgcagcct |
| | KYYADSVKGRFTISRDNSKNTLYLQM | ggaaggagcctgagactctcatgcgccgcgtcagg |
| | NSLRAEDTAVYYCGGSGYALHDDYYG | gttcaccttttcctcctacgggatgcattgggtca |
| | LDVWGQGTLVTVSSGGGGSGGGGSGG | gacaggcccccggaaagggactcgaatgggtggct |
| | GGSQSALTQPASVSGSPGQSITISCT | gtgatcagctacgacggctccaacaagtactacgc |
| | GTSSDVGGYNYVSWYQQHPGKAPKLM | cgactccgtgaaaggccggttcactatctcccggg |
| | IYDVSNRPSGVSNRFSGSKSGNTASL | acaactccaagaacacgctgtatctgcaaatgaat |
| | TISGLQAEDEADYYCSSYTSSSTLYV | tcactgcgcgcggaggataccgctgtgtactactg |
| | FGSGTKVTVLTTTPAPRPPTPAPTIA | cggtggctccggttacgccctgcacgatgactatt |
| | SQPLSLRPEACRPAAGGAVHTRGLDF | acggccttgacgtctggggccagggaaccctcgtg |
| | ACDIYIWAPLAGTCGVLLLSLVITLY | actgtgtccagcggtggaggaggttcgggcggagg |
| | CKRGRKKLLYIFKQPFMRPVQTTQEE | aggatcaggagggggtggatcgcagagcgcactga |
| | DGCSCRFPEEEEGGCELRVKFSRSAD | ctcagccggcatccgtgtccggtagccccggacag |
| | APAYQQGQNQLYNELNLGRREEYDVL | tcgattaccatctcctgtaccggcacctcctccga |
| | DKRRGRDPEMGGKPRRKNPQEGLYNE | cgtgggagggtacaactacgtgtcgtggtaccagc |
| | LQKDKMAEAYSEIGMKGERRRGKGHD | agcacccaggaaaggcccctaagttgatgatctac |
| | GLYQGLSTATKDTYDALHMQALPPRG | gatgtgtcaaaccgcccgtctggagtctccaaccg |
| | SGATNFSLLKQAGDVEENPGPMALPV | gttctccggctccaagtccggcaacaccgccagcc |
| | TALLLPLALLLHAARPEIVMTQSPAT | tgaccattagcgggctgcaagccgaggatgaggcc |
| | LSLSPGERATLSCRASQDISKYLNWY | gactactactgctcgagctacacatcctcgagcac |
| | QQKPGQAPRLLIYHTSRLHSGIPARF | cctctacgtgttcggctcggggactaaggtcaccg |
| | SGSGSGTDYTLTISSLQPEDFAVYFC | tgctgaccactaccccagcaccgaggccacccacc |
| | QQGNTLPYTFGQGTKLEIKGGGGSGG | ccggctcctaccatcgcctcccagcctctgtccct |
| | GGSGGGGSQVQLQESGPGLVKPSETL | gcgtccggaggcatgtagacccgcagctggtgggg |
| | SLTCTVSGVSLPDYGVSWIRQPPGKG | ccgtgcatacccggggtcttgacttcgcctgcgat |
| | LEWIGVIWGSETTYYQSSLKSRVTIS | atctacatttgggcccctctggctggtacttgcgg |
| | KDNSKNQVSLKLSSVTAADTAVYYCA | ggtcctgctgctttcactcgtgatcactctttact |
| | KHYYYGGSYAMDYWGQGTLVTVSSTT | gtaagcgcggtcggaagaagctgctgtacatcttt |
| | TPAPRPPTPAPTIASQPLSLRPEACR | aagcaacccttcatgaggcctgtgcagactactca |
| | PAAGGAVHTRGLDFACDIYIWAPLAG | agaggaggacggctgttcatgccggttcccagagg |
| | TCGVLLLSLVITLYCKRGRKKLLYIF | aggaggaaggcggctgcgaactgcgcgtgaaattc |
| | KQPFMRPVQTTQEEDGCSCRFPEEEE | agccgcagcgcagatgctccagcctaccagcaggg |
| | GGCELRVKFSRSADAPAYQQGQNQLY | gcagaaccagctctacaacgaactcaatcttggtc |
| | NELNLGRREEYDVLDKRRGRDPEMGG | ggagagaggagtacgacgtgctggacaagcggaga |
| | KPRRKNPQEGLYNELQKDKMAEAYSE | ggacgggacccagaaatgggcgggaagccgcgcag |
| | IGMKGERRRGKGHDGLYQGLSTATKD | aaagaatccccaagagggcctgtacaacgagctcc |
| | TYDALHMQALPPR (SEQ ID NO: | aaaaggataagatggcagaagcctatagcgagatt |
| | 216) | ggtatgaaaggggaacgcagaagaggcaaaggcca |
| | | cgacggactgtaccagggactcagcaccgccacca |
| | | aggacacctatgacgctcttcacatgcaggccctg |
| | | ccgcctcggggaagcggagctactaacttcagcct |
| | | gctgaagcaggctggagacgtggaggagaaccctg |
| | | gacctatggccttaccagtgaccgccttgctcctg |
| | | ccgctggccttgctgctccacgccgccaggccgga |
| | | aattgtgatgacccagtcacccgccactcttagcc |
| | | tttcacccggtgagcgcgcaaccctgtcttgcaga |
| | | gcctoccaagacatctcaaaataccttaattggta |
| | | tcaacagaagcccggacaggctcctcgccttctga |
| | | tctaccacaccagccggctccattctggaatccct |
| | | gccaggttcagcggtagcggatctgggaccgacta |
| | | caccctcactatcagctcactgcagccagaggact |
| | | tcgctgtctatttctgtcagcaagggaacaccctg |
| | | ccctacacctttggacagggcaccaagctcgagat |
| | | taaaggtggaggtggcagcggaggaggtgggtccg |
| | | gcggtggaggaagccaggtccaactccaagaaagc |
| | | ggaccgggtcttgtgaagccatcagaaactctttc |
| | | actgacttgtactgtgagcggagtgtctctccccg |
| | | attacggggtgtcttggatcagacagccaccgggg |
| | | aagggtctggaatggattggagtgatttggggctc |
| | | tgagactacttactaccaatcatccctcaagtcac |
| | | gcgtcaccatctcaaaggacaactctaagaatcag |
| | | gtgtcactgaaactgtcatctgtgaccgcagccga |
| | | caccgccgtgtactattgcgctaagcattactatt |
| | | atggcgggagctacgcaatggattactggggacag |
| | | ggtactctggtcaccgtgtccagcaccacgacgcc |
| | | agcgccgcgaccaccaacaccggcgcccaccatcg |
| | | cgtcgcagcccctgtccctgcgcccagaggcgtgc |
| | | cggccagcggcggggggcgcagtgcacacgagggg |
| | | gctggacttcgcctgtgatatctacatctgggcgc |
| | | ccttggccgggacttgtggggtccttctcctgtca |
| | | ctggttatcaccctttactgcaaacggggcagaaa |
| | | gaaactcctgtatatattcaaacaaccatttatga |
| | | gaccagtacaaactactcaagaggaagatggctgt |
| | | agctgccgatttccagaagaagaagaaggaggatg |
| | | tgaactgagagtgaagttcagcaggagcgcagacg |
| | | cccccgcgtaccagcagggccagaaccagctctat |
| | | aacgagctcaatctaggacgaagagaggagtacga |
| | | tgttttggacaagagacgtggccgggaccctgaga |
| | | tggggggaaagccgagaaggaagaaccctcaggaa |
| | | ggcctgtacaatgaactgcagaaagataagatggc |
| | | ggaggcctacagtgagattgggatgaaaggcgagc |
| | | gccggaggggcaaggggcacgatggcctttaccag |
| | | ggtctcagtacagccaccaaggacacctacgacgc |
| | | ccttcacatgcaggccctgccccctcgc (SEQ |
| | | ID NO: 217) |
| R1B6-P2A- | MALPVTALLLPLALLLHAARPEVQLL | atggccctccctgtcaccgccctgctgcttccgct |
| duCD19.1 | ESGGGLVQPGGSLRLSCAASGFTFSS | ggctcttctgctccacgccgctcggcccgaagtgc |
| | YAMSWVRQAPGKGLEWVSAISGSGGS | agttgctggagtcaggcggaggactggtgcagccc |
| | TYYADSVKGRFTISRDNSKNTLYLQM | ggaggatcgcttcgcttgagctgcgcagcctcagg |
| | NSLRAEDTAVYYCARREWVPYDVSWY | ctttaccttctcctcctacgccatgtcctgggtca |
| | FDYWGQGTLVTVSSGGGGSGGGGSGG | gacaggctcccgggaagggactggaatgggtgtcc |
| | GGSGGGGSDIQMTQSPSSLSASVGDR | gccattagcggttccggcggaagcacttactatgc |
| | VTITCRASQSISSYLNWYQQKPGKAP | cgactctgtgaagggccgcttcactatctcccggg |
| | KLLIYAASSLQSGVPSRFSGSGSGTD | acaactccaagaacaccctgtatctccaaatgaat |
| | FTLTISSLQPEDFATYYCQQSYSTPL | tccctgagggccgaagataccgcggtgtactactg |
| | TFGQGTKVEIKTTTPAPRPPTPAPTI | cgctagacgggagtgggtgccctacgatgtcagct |
| | ASQPLSLRPEACRPAAGGAVHTRGLD | ggtacttcgactactggggacagggcactctcgtg |
| | FACDIYIWAPLAGTCGVLLLSLVITL | actgtgtcctccggtggtggtggatcggggggtgg |
| | YCKRGRKKLLYIFKQPFMRPVQTTQE | tggttcgggcggaggaggatctggaggaggagggt |
| | EDGCSCRFPEEEEGGCELRVKFSRSA | cggacattcaaatgactcagtccccgtcctccctc |
| | DAPAYQQGQNQLYNELNLGRREEYDV | tccgcctccgtgggagatcgcgtcacgatcacgtg |
| | LDKRRGRDPEMGGKPRRKNPQEGLYN | cagggccagccagagcatctccagctacctgaact |
| | ELQKDKMAEAYSEIGMKGERRRGKGH | ggtaccagcagaagccagggaaggcaccgaagctc |
| | DGLYQGLSTATKDTYDALHMQALPPR | ctgatctacgccgctagctcgctgcagtccggcgt |
| | GSGATNFSLLKQAGDVEENPGPMALP | cccttcacggttctcgggatcgggctcaggcaccg |
| | VTALLLPLALLLHAARPEIVMTQSPA | acttcaccctgaccattagcagcctgcagccggag |
| | TLSLSPGERATLSCRASQDISKYLNW | gacttcgcgacatactactgtcagcagtcatactc |
| | YQQKPGQAPRLLIYHTSRLHSGIPAR | cacccctctgaccttcggccaagggaccaaagtgg |
| | FSGSGSGTDYTLTISSLQPEDFAVYF | agatcaagaccactaccccagcaccgaggccaccc |
| | CQQGNTLPYTFGQGTKLEIKGGGGSG | accccggctcctaccatcgcctcccagcctctgtc |
| | GGGSGGGGSQVQLQESGPGLVKPSET | cctgcgtccggaggcatgtagacccgcagctggtg |
| | LSLTCTVSGVSLPDYGVSWIRQPPGK | gggccgtgcatacccggggtcttgacttcgcctgc |
| | GLEWIGVIWGSETTYYQSSLKSRVTI | gatatctacatttgggcccctctggctggtacttg |
| | SKDNSKNQVSLKLSSVTAADTAVYYC | cggggtcctgctgctttcactcgtgatcactcttt |
| | AKHYYYGGSYAMDYWGQGTLVTVSST | actgtaagcgcggtcggaagaagctgctgtacatc |
| | TTPAPRPPTPAPTIASQPLSLRPEAC | tttaagcaacccttcatgaggcctgtgcagactac |
| | RPAAGGAVHTRGLDFACDIYIWAPLA | tcaagaggaggacggctgttcatgccggttcccag |
| | GTCGVLLLSLVITLYCKRGRKKLLYI | aggaggaggaaggcggctgcgaactgcgcgtgaaa |
| | FKQPFMRPVQTTQEEDGCSCRFPEEE | ttcagccgcagcgcagatgctccagcctaccagca |
| | EGGCELRVKFSRSADAPAYQQGQNQL | ggggcagaaccagctctacaacgaactcaatcttg |
| | YNELNLGRREEYDVLDKRRGRDPEMG | gtcggagagaggagtacgacgtgctggacaagcgg |
| | GKPRRKNPQEGLYNELQKDKMAEAYS | agaggacgggacccagaaatgggcgggaagccgcg |
| | EIGMKGERRRGKGHDGLYQGLSTATK | cagaaagaatccccaagagggcctgtacaacgagc |
| | DTYDALHMQALPPR (SEQ ID NO: | tccaaaaggataagatggcagaagcctatagcgag |
| | 218) | attggtatgaaaggggaacgcagaagaggcaaagg |
| | | ccacgacggactgtaccagggactcagcaccgcca |
| | | ccaaggacacctatgacgctcttcacatgcaggcc |
| | | ctgccgcctcggggaagcggagctactaacttcag |
| | | cctgctgaagcaggctggagacgtggaggagaacc |
| | | ctggacctatggccttaccagtgaccgccttgctc |
| | | ctgccgctggccttgctgctccacgccgccaggcc |
| | | ggaaattgtgatgacccagtcacccgccactctta |
| | | gcctttcacccggtgagcgcgcaaccctgtcttgc |
| | | agagcctcccaagacatctcaaaataccttaattg |
| | | gtatcaacagaagcccggacaggctcctcgccttc |
| | | tgatctaccacaccagccggctccattctggaatc |
| | | cctgccaggttcagcggtagcggatctgggaccga |
| | | ctacaccctcactatcagctcactgcagccagagg |
| | | acttcgctgtctatttctgtcagcaagggaacacc |
| | | ctgccctacacctttggacagggcaccaagctcga |
| | | gattaaaggtggaggtggcagcggaggaggtgggt |
| | | ccggcggtggaggaagccaggtccaactccaagaa |
| | | agcggaccgggtcttgtgaagccatcagaaactct |
| | | ttcactgacttgtactgtgagcggagtgtctctcc |
| | | ccgattacggggtgtcttggatcagacagccaccg |
| | | gggaagggtctggaatggattggagtgatttgggg |
| | | ctctgagactacttactaccaatcatccctcaagt |
| | | cacgcgtcaccatctcaaaggacaactctaagaat |
| | | caggtgtcactgaaactgtcatctgtgaccgcagc |
| | | cgacaccgccgtgtactattgcgctaagcattact |
| | | attatggcgggagctacgcaatggattactgggga |
| | | cagggtactctggtcaccgtgtccagcaccacgac |
| | | gccagcgccgcgaccaccaacaccggcgcccacca |
| | | tcgcgtcgcagcccctgtccctgcgcccagaggcg |
| | | tgccggccagcggcggggggcgcagtgcacacgag |
| | | ggggctggacttcgcctgtgatatctacatctggg |
| | | cgcccttggccgggacttgtggggtccttctcctg |
| | | tcactggttatcaccctttactgcaaacggggcag |
| | | aaagaaactcctgtatatattcaaacaaccattta |
| | | tgagaccagtacaaactactcaagaggaagatggc |
| | | tgtagctgccgatttccagaagaagaagaaggagg |
| | | atgtgaactgagagtgaagttcagcaggagcgcag |
| | | acgcccccgcgtaccagcagggccagaaccagctc |
| | | tataacgagctcaatctaggacgaagagaggagta |
| | | cgatgttttggacaagagacgtggccgggaccctg |
| | | agatggggggaaagccgagaaggaagaaccctcag |
| | | gaaggcctgtacaatgaactgcagaaagataagat |
| | | ggcggaggcctacagtgagattgggatgaaaggcg |
| | | agcgccggaggggcaaggggcacgatggcctttac |
| | | cagggtctcagtacagccaccaaggacacctacga |
| | | cgcccttcacatgcaggccctgccccctcgc |
| | | (SEQ ID NO: 219) |
| duBCMA.4-P2A- | MALPVTALLLPLALLLHAARPEVQLV | atggccctccctgtcaccgccctgctgcttccgct |
| duCD19.1 | ESGGGLVQPGGSLRLSCAVSGFALSN | ggctcttctgctccacgccgctcggcccgaagtgc |
| | HGMSWVRRAPGKGLEWVSGIVYSGST | aattggtggaatcagggggaggacttgtgcagcct |
| | YYAASVKGRFTISRDNSRNTLYLQMN | ggaggatcgctgagactgtcatgtgccgtgtccgg |
| | SLRPEDTAIYYCSAHGGESDVWGQGT | ctttgccctgtccaaccacgggatgtcctgggtcc |
| | TVTVSSASGGGGSGGGGSGGGGSDIQ | gccgcgcgcctggaaagggcctcgaatgggtgtcg |
| | LTQSPSSLSASVGDRVTITCRASQSI | ggtattgtgtacagcggtagcacctactatgccgc |
| | SSYLNWYQQKPGKAPKLLIYAASSLQ | atccgtgaaggggagattcaccatcagccgggaca |
| | SGVPSRFSGSGSGTDFTLTISSLQPE | actccaggaacactctgtacctccaaatgaattcg |
| | DFATYYCQQSYSTPYTFGQGTKVEIK | ctgaggccagaggacactgccatctactactgctc |
| | TTTPAPRPPTPAPTIASQPLSLRPEA | cgcgcatggcggagagtccgacgtctggggacagg |
| | CRPAAGGAVHTRGLDFACDIYIWAPL | ggaccaccgtgaccgtgtctagcgcgtccggcgga |
| | AGTCGVLLLSLVITLYCKRGRKKLLY | ggcggcagcgggggtggtggttcagggggcggcgg |
| | IFKQPFMRPVQTTQEEDGCSCRFPEE | atcggacatccagctcacccagtccccgagctcgc |
| | EEGGCELRVKFSRSADAPAYQQGQNQ | tgtccgcctccgtgggagatcgggtcaccatcacg |
| | LYNELNLGRREEYDVLDKRRGRDPEM | tgccgcgccagccagtcgatttcctcctacctgaa |
| | GGKPRRKNPQEGLYNELQKDKMAEAY | ctggtaccaacagaagcccggaaaagccccgaagc |
| | SEIGMKGERRRGKGHDGLYQGLSTAT | ttctcatctacgccgcctcgagcctgcagtcagga |
| | KDTYDALHMQALPPRGSGATNFSLLK | gtgccctcacggttctccggctccggttccggtac |
| | QAGDVEENPGPMALPVTALLLPLALL | tgatttcaccctgaccatttcctccctgcaaccgg |
| | LHAARPEIVMTQSPATLSLSPGERAT | aggacttcgctacttactactgccagcagtcgtac |
| | LSCRASQDISKYLNWYQQKPGQAPRL | tccaccccctacactttcggacaaggcaccaaggt |
| | LIYHTSRLHSGIPARFSGSGSGTDYT | cgaaatcaagaccactaccccagcaccgaggccac |
| | LTISSLQPEDFAVYFCQQGNTLPYTF | ccaccccggctcctaccatcgcctcccagcctctg |
| | GQGTKLEIKGGGGSGGGGSGGGGSQV | tccctgcgtccggaggcatgtagacccgcagctgg |
| | QLQESGPGLVKPSETLSLTCTVSGVS | tggggccgtgcatacccggggtcttgacttcgcct |
| | LPDYGVSWIRQPPGKGLEWIGVIWGS | gcgatatctacatttgggcccctctggctggtact |
| | ETTYYQSSLKSRVTISKDNSKNQVSL | tgcggggtcctgctgctttcactcgtgatcactct |
| | KLSSVTAADTAVYYCAKHYYYGGSYA | ttactgtaagcgcggtcggaagaagctgctgtaca |
| | MDYWGQGTLVTVSSTTTPAPRPPTPA | tctttaagcaacccttcatgaggcctgtgcagact |
| | PTIASQPLSLRPEACRPAAGGAVHTR | actcaagaggaggacggctgttcatgccggttccc |
| | GLDFACDIYIWAPLAGTCGVLLLSLV | agaggaggaggaaggcggctgcgaactgcgcgtga |
| | ITLYCKRGRKKLLYIFKQPFMRPVQT | aattcagccgcagcgcagatgctccagcctaccag |
| | TQEEDGCSCRFPEEEEGGCELRVKES | caggggcagaaccagctctacaacgaactcaatct |
| | RSADAPAYQQGQNQLYNELNLGRREE | tggtcggagagaggagtacgacgtgctggacaagc |
| | YDVLDKRRGRDPEMGGKPRRKNPQEG | ggagaggacgggacccagaaatgggcgggaagccg |
| | LYNELQKDKMAEAYSEIGMKGERRRG | cgcagaaagaatccccaagagggcctgtacaacga |
| | KGHDGLYQGLSTATKDTYDALHMQAL | gctccaaaaggataagatggcagaagcctatagcg |
| | PPR (SEQ ID NO: 220) | agattggtatgaaaggggaacgcagaagaggcaaa |
| | | ggccacgacggactgtaccagggactcagcaccgc |
| | | caccaaggacacctatgacgctcttcacatgcagg |
| | | ccctgccgcctcggggaagcggagctactaacttc |
| | | agcctgctgaagcaggctggagacgtggaggagaa |
| | | ccctggacctatggccttaccagtgaccgccttgc |
| | | tcctgccgctggccttgctgctccacgccgccagg |
| | | ccggaaattgtgatgacccagtcacccgccactct |
| | | tagcctttcacccggtgagcgcgcaaccctgtctt |
| | | gcagagcctoccaagacatctcaaaataccttaat |
| | | tggtatcaacagaagcccggacaggctcctcgcct |
| | | tctgatctaccacaccagccggctccattctggaa |
| | | tccctgccaggttcagcggtagcggatctgggacc |
| | | gactacaccctcactatcagctcactgcagccaga |
| | | ggacttcgctgtctatttctgtcagcaagggaaca |
| | | ccctgccctacacctttggacagggcaccaagctc |
| | | gagattaaaggtggaggtggcagcggaggaggtgg |
| | | gtccggcggtggaggaagccaggtccaactccaag |
| | | aaagcggaccgggtcttgtgaagccatcagaaact |
| | | ctttcactgacttgtactgtgagcggagtgtctct |
| | | ccccgattacggggtgtcttggatcagacagccac |
| | | cggggaagggtctggaatggattggagtgatttgg |
| | | ggctctgagactacttactaccaatcatccctcaa |
| | | gtcacgcgtcaccatctcaaaggacaactctaaga |
| | | atcaggtgtcactgaaactgtcatctgtgaccgca |
| | | gccgacaccgccgtgtactattgcgctaagcatta |
| | | ctattatggcgggagctacgcaatggattactggg |
| | | gacagggtactctggtcaccgtgtccagcaccacg |
| | | acgccagcgccgcgaccaccaacaccggcgcccac |
| | | catcgcgtcgcagcccctgtccctgcgcccagagg |
| | | cgtgccggccagcggcggggggcgcagtgcacacg |
| | | agggggctggacttcgcctgtgatatctacatctg |
| | | ggcgcccttggccgggacttgtggggtccttctcc |
| | | tgtcactggttatcaccctttactgcaaacggggc |
| | | agaaagaaactcctgtatatattcaaacaaccatt |
| | | tatgagaccagtacaaactactcaagaggaagatg |
| | | gctgtagctgccgatttccagaagaagaagaagga |
| | | ggatgtgaactgagagtgaagttcagcaggagcgc |
| | | agacgcccccgcgtaccagcagggccagaaccagc |
| | | tctataacgagctcaatctaggacgaagagaggag |
| | | tacgatgttttggacaagagacgtggccgggaccc |
| | | tgagatggggggaaagccgagaaggaagaaccctc |
| | | aggaaggcctgtacaatgaactgcagaaagataag |
| | | atggcggaggcctacagtgagattgggatgaaagg |
| | | cgagcgccggaggggcaaggggcacgatggccttt |
| | | accagggtctcagtacagccaccaaggacacctac |
| | | gacgcccttcacatgcaggccctgccccctcgc |
| | | (SEQ ID NO: 221) |
| duCD19.1-P2A- | MALPVTALLLPLALLLHAARPEIVMT | atggccttaccagtgaccgccttgctcctgccgct |
| duBCMA.4 | QSPATLSLSPGERATLSCRASQDISK | ggccttgctgctccacgccgccaggccggaaattg |
| | YLNWYQQKPGQAPRLLIYHTSRLHSG | tgatgacccagtcacccgccactcttagcctttca |
| | IPARFSGSGSGTDYTLTISSLQPEDF | cccggtgagcgcgcaaccctgtcttgcagagcctc |
| | AVYFCQQGNTLPYTFGQGTKLEIKGG | ccaagacatctcaaaataccttaattggtatcaac |
| | GGSGGGGSGGGGSQVQLQESGPGLVK | agaagcccggacaggctcctcgccttctgatctac |
| | PSETLSLTCTVSGVSLPDYGVSWIRQ | cacaccagccggctccattctggaatccctgccag |
| | PPGKGLEWIGVIWGSETTYYQSSLKS | gttcagcggtagcggatctgggaccgactacaccc |
| | RVTISKDNSKNQVSLKLSSVTAADTA | tcactatcagctcactgcagccagaggacttcgct |
| | VYYCAKHYYYGGSYAMDYWGQGTLVT | gtctatttctgtcagcaagggaacaccctgcccta |
| | VSSTTTPAPRPPTPAPTIASQPLSLR | cacctttggacagggcaccaagctcgagattaaag |
| | PEACRPAAGGAVHTRGLDFACDIYIW | gtggaggtggcagcggaggaggtgggtccggcggt |
| | APLAGTCGVLLLSLVITLYCKRGRKK | ggaggaagccaggtccaactccaagaaagcggacc |
| | LLYIFKQPFMRPVQTTQEEDGCSCRF | gggtcttgtgaagccatcagaaactctttcactga |
| | PEEEEGGCELRVKFSRSADAPAYQQG | cttgtactgtgagcggagtgtctctccccgattac |
| | QNQLYNELNLGRREEYDVLDKRRGRD | ggggtgtcttggatcagacagccaccggggaaggg |
| | PEMGGKPRRKNPQEGLYNELQKDKMA | tctggaatggattggagtgatttggggctctgaga |
| | EAYSEIGMKGERRRGKGHDGLYQGLS | ctacttactaccaatcatccctcaagtcacgcgtc |
| | TATKDTYDALHMQALPPRGSGATNFS | accatctcaaaggacaactctaagaatcaggtgtc |
| | LLKQAGDVEENPGPMALPVTALLLPL | actgaaactgtcatctgtgaccgcagccgacaccg |
| | ALLLHAARPEVQLVESGGGLVQPGGS | ccgtgtactattgcgctaagcattactattatggc |
| | LRLSCAVSGFALSNHGMSWVRRAPGK | gggagctacgcaatggattactggggacagggtac |
| | GLEWVSGIVYSGSTYYAASVKGRFTI | tctggtcaccgtgtccagcaccacgacgccagcgc |
| | SRDNSRNTLYLQMNSLRPEDTAIYYC | cgcgaccaccaacaccggcgcccaccatcgcgtcg |
| | SAHGGESDVWGQGTTVTVSSASGGGG | cagcccctgtccctgcgcccagaggcgtgccggcc |
| | SGGGGSGGGGSDIQLTQSPSSLSASV | agcggcggggggcgcagtgcacacgagggggctgg |
| | GDRVTITCRASQSISSYLNWYQQKPG | acttcgcctgtgatatctacatctgggcgcccttg |
| | KAPKLLIYAASSLQSGVPSRFSGSGS | gccgggacttgtggggtccttctcctgtcactggt |
| | GTDFTLTISSLQPEDFATYYCQQSYS | tatcaccctttactgcaaacggggcagaaagaaac |
| | TPYTFGQGTKVEIKTTTPAPRPPTPA | tcctgtatatattcaaacaaccatttatgagacca |
| | PTIASQPLSLRPEACRPAAGGAVHTR | gtacaaactactcaagaggaagatggctgtagctg |
| | GLDFACDIYIWAPLAGTCGVLLLSLV | ccgatttccagaagaagaagaaggaggatgtgaac |
| | ITLYCKRGRKKLLYIFKQPFMRPVQT | tgagagtgaagttcagcaggagcgcagacgccccc |
| | TQEEDGCSCRFPEEEEGGCELRVKES | gcgtaccagcagggccagaaccagctctataacga |
| | RSADAPAYQQGQNQLYNELNLGRREE | gctcaatctaggacgaagagaggagtacgatgttt |
| | YDVLDKRRGRDPEMGGKPRRKNPQEG | tggacaagagacgtggccgggaccctgagatgggg |
| | LYNELQKDKMAEAYSEIGMKGERRRG | ggaaagccgagaaggaagaaccctcaggaaggect |
| | KGHDGLYQGLSTATKDTYDALHMQAL | gtacaatgaactgcagaaagataagatggcggagg |
| | PPR (SEQ ID NO: 222) | cctacagtgagattgggatgaaaggcgagcgccgg |
| | | aggggcaaggggcacgatggcctttaccagggtct |
| | | cagtacagccaccaaggacacctacgacgcccttc |
| | | acatgcaggccctgccccctcgcggaagcggagct |
| | | actaacttcagcctgctgaagcaggctggagacgt |
| | | ggaggagaaccctggacctatggccctccctgtca |
| | | ccgccctgctgcttccgctggctcttctgctccac |
| | | gccgctcggcccgaagtgcaattggtggaatcagg |
| | | gggaggacttgtgcagcctggaggatcgctgagac |
| | | tgtcatgtgccgtgtccggctttgccctgtccaac |
| | | cacgggatgtcctgggtccgccgcgcgcctggaaa |
| | | gggcctcgaatgggtgtcgggtattgtgtacagcg |
| | | gtagcacctactatgccgcatccgtgaaggggaga |
| | | ttcaccatcagccgggacaactccaggaacactct |
| | | gtacctccaaatgaattcgctgaggccagaggaca |
| | | ctgccatctactactgctccgcgcatggcggagag |
| | | tccgacgtctggggacaggggaccaccgtgaccgt |
| | | gtctagcgcgtccggcggaggcggcagcgggggtg |
| | | gtggttcagggggcggcggatcggacatccagctc |
| | | acccagtccccgagctcgctgtccgcctccgtggg |
| | | agatcgggtcaccatcacgtgccgcgccagccagt |
| | | cgatttcctcctacctgaactggtaccaacagaag |
| | | cccggaaaagccccgaagcttctcatctacgccgc |
| | | ctcgagcctgcagtcaggagtgccctcacggttct |
| | | ccggctccggttccggtactgatttcaccctgacc |
| | | atttcctccctgcaaccggaggacttcgctactta |
| | | ctactgccagcagtcgtactccaccccctacactt |
| | | tcggacaaggcaccaaggtcgaaatcaagaccact |
| | | accccagcaccgaggccacccaccccggctcctac |
| | | catcgcctcccagcctctgtccctgcgtccggagg |
| | | catgtagacccgcagctggtggggccgtgcatacc |
| | | cggggtcttgacttcgcctgcgatatctacatttg |
| | | ggcccctctggctggtacttgcggggtcctgctgc |
| | | tttcactcgtgatcactctttactgtaagcgcggt |
| | | cggaagaagctgctgtacatctttaagcaaccctt |
| | | catgaggcctgtgcagactactcaagaggaggacg |
| | | gctgttcatgccggttcccagaggaggaggaaggc |
| | | ggctgcgaactgcgcgtgaaattcagccgcagcgc |
| | | agatgctccagcctaccagcaggggcagaaccagc |
| | | tctacaacgaactcaatcttggtcggagagaggag |
| | | tacgacgtgctggacaagcggagaggacgggaccc |
| | | agaaatgggcgggaagccgcgcagaaagaatcccc |
| | | aagagggcctgtacaacgagctccaaaaggataag |
| | | atggcagaagcctatagcgagattggtatgaaagg |
| | | ggaacgcagaagaggcaaaggccacgacggactgt |
| | | accagggactcagcaccgccaccaaggacacctat |
| | | gacgctcttcacatgcaggccctgccgcctcgg |
| | | (SEQ ID NO: 223) |
| Predicted | Anti-BCMA CAR arm: | |
| resultant | EVQLLESGGGLVQPGGSLRLSCAASG | |
| proteins from | FTFSSYAMSWVRQAPGKGLEWVSAIS | |
| R1G5-P2A- | GSGGSTYYADSVKGRFTISRDNSKNT | |
| duCD19.1 | LYLQMNSLRAEDTAVYYCARREWWGE | |
| | SWLFDYWGQGTLVTVSSGGGGSGGGG | |
| | SGGGGSGGGGSDIQMTQSPSSLSASV | |
| | GDRVTITCRASQSISSYLNWYQQKPG | |
| | KAPKLLIYAASSLQSGVPSRFSGSGS | |
| | GTDFTLTISSLQPEDFATYYCQQSYS | |
| | TPLTFGQGTKVEIKTTTPAPRPPTPA | |
| | PTIASQPLSLRPEACRPAAGGAVHTR | |
| | GLDFACDIYIWAPLAGTCGVLLLSLV | |
| | ITLYCKRGRKKLLYIFKQPFMRPVQT | |
| | TQEEDGCSCRFPEEEEGGCELRVKES | |
| | RSADAPAYQQGQNQLYNELNLGRREE | |
| | YDVLDKRRGRDPEMGGKPRRKNPQEG | |
| | LYNELQKDKMAEAYSEIGMKGERRRG | |
| | KGHDGLYQGLSTATKDTYDALHMQAL | |
| | PPRGSGATNFSLLKQAGDVEENPG | |
| | (SEQ ID NO: 224) | |
| | Anti-CD19 CAR arm: | |
| | EIVMTQSPATLSLSPGERATLSCRAS | |
| | QDISKYLNWYQQKPGQAPRLLIYHTS | |
| | RLHSGIPARFSGSGSGTDYTLTISSL | |
| | QPEDFAVYFCQQGNTLPYTFGQGTKL | |
| | EIKGGGGSGGGGSGGGGSQVQLQESG | |
| | PGLVKPSETLSLTCTVSGVSLPDYGV | |
| | SWIRQPPGKGLEWIGVIWGSETTYYQ | |
| | SSLKSRVTISKDNSKNQVSLKLSSVT | |
| | AADTAVYYCAKHYYYGGSYAMDYWGQ | |
| | GTLVTVSSTTTPAPRPPTPAPTIASQ | |
| | PLSLRPEACRPAAGGAVHTRGLDFAC | |
| | DIYIWAPLAGTCGVLLLSLVITLYCK | |
| | RGRKKLLYIFKQPFMRPVQTTQEEDG | |
| | CSCRFPEEEEGGCELRVKFSRSADAP | |
| | AYQQGQNQLYNELNLGRREEYDVLDK | |
| | RRGRDPEMGGKPRRKNPQEGLYNELQ | |
| | KDKMAEAYSEIGMKGERRRGKGHDGL | |
| | YQGLSTATKDTYDALHMQALPPR | |
| | (SEQ ID NO: 225) | |
| Predicted | Anti-BCMA CAR arm: | |
| resultant | QVQLQESGGGVVQPGRSLRLSCAASG | |
| proteins from | FTFSSYGMHWVRQAPGKGLEWVAVIS | |
| PI61-P2A- | YDGSNKYYADSVKGRFTISRDNSKNT | |
| duCD19.1 | LYLQMNSLRAEDTAVYYCGGSGYALH | |
| | DDYYGLDVWGQGTLVTVSSGGGGSGG | |
| | GGSGGGGSQSALTQPASVSGSPGQSI | |
| | TISCTGTSSDVGGYNYVSWYQQHPGK | |
| | APKLMIYDVSNRPSGVSNRFSGSKSG | |
| | NTASLTISGLQAEDEADYYCSSYTSS | |
| | STLYVFGSGTKVTVLTTTPAPRPPTP | |
| | APTIASQPLSLRPEACRPAAGGAVHT | |
| | RGLDFACDIYIWAPLAGTCGVLLLSL | |
| | VITLYCKRGRKKLLYIFKQPFMRPVQ | |
| | TTQEEDGCSCRFPEEEEGGCELRVKF | |
| | SRSADAPAYQQGQNQLYNELNLGRRE | |
| | EYDVLDKRRGRDPEMGGKPRRKNPQE | |
| | GLYNELQKDKMAEAYSEIGMKGERRR | |
| | GKGHDGLYQGLSTATKDTYDALHMQA | |
| | LPPRGSGATNFSLLKQAGDVEENPG | |
| | (SEQ ID NO: 226) | |
| | Anti-CD19 CAR arm: | |
| | EIVMTQSPATLSLSPGERATLSCRAS | |
| | QDISKYLNWYQQKPGQAPRLLIYHTS | |
| | RLHSGIPARFSGSGSGTDYTLTISSL | |
| | QPEDFAVYFCQQGNTLPYTFGQGTKL | |
| | EIKGGGGSGGGGSGGGGSQVQLQESG | |
| | PGLVKPSETLSLTCTVSGVSLPDYGV | |
| | SWIRQPPGKGLEWIGVIWGSETTYYQ | |
| | SSLKSRVTISKDNSKNQVSLKLSSVT | |
| | AADTAVYYCAKHYYYGGSYAMDYWGQ | |
| | GTLVTVSSTTTPAPRPPTPAPTIASQ | |
| | PLSLRPEACRPAAGGAVHTRGLDFAC | |
| | DIYIWAPLAGTCGVLLLSLVITLYCK | |
| | RGRKKLLYIFKQPFMRPVQTTQEEDG | |
| | CSCRFPEEEEGGCELRVKFSRSADAP | |
| | AYQQGQNQLYNELNLGRREEYDVLDK | |
| | RRGRDPEMGGKPRRKNPQEGLYNELQ | |
| | KDKMAEAYSEIGMKGERRRGKGHDGL | |
| | YQGLSTATKDTYDALHMQALPPR | |
| | (SEQ ID NO: 225) | |
| Predicted | Anti-BCMA CAR arm: | |
| resultant | EVQLLESGGGLVQPGGSLRLSCAASG | |
| proteins from | FTFSSYAMSWVRQAPGKGLEWVSAIS | |
| R1B6-P2A- | GSGGSTYYADSVKGRFTISRDNSKNT | |
| duCD19.1 | LYLQMNSLRAEDTAVYYCARREWVPY | |
| | DVSWYFDYWGQGTLVTVSSGGGGSGG | |
| | GGSGGGGSGGGGSDIQMTQSPSSLSA | |
| | SVGDRVTITCRASQSISSYLNWYQQK | |
| | PGKAPKLLIYAASSLQSGVPSRFSGS | |
| | GSGTDFTLTISSLQPEDFATYYCQQS | |
| | YSTPLTFGQGTKVEIKTTTPAPRPPT | |
| | PAPTIASQPLSLRPEACRPAAGGAVH | |
| | TRGLDFACDIYIWAPLAGTCGVLLLS | |
| | LVITLYCKRGRKKLLYIFKQPFMRPV | |
| | QTTQEEDGCSCRFPEEEEGGCELRVK | |
| | FSRSADAPAYQQGQNQLYNELNLGRR | |
| | EEYDVLDKRRGRDPEMGGKPRRKNPQ | |
| | EGLYNELQKDKMAEAYSEIGMKGERR | |
| | RGKGHDGLYQGLSTATKDTYDALHMQ | |
| | ALPPRGSGATNFSLLKQAGDVEENPG | |
| | (SEQ ID NO: 227) | |
| | Anti-CD19 CAR arm: | |
| | EIVMTQSPATLSLSPGERATLSCRAS | |
| | QDISKYLNWYQQKPGQAPRLLIYHTS | |
| | RLHSGIPARFSGSGSGTDYTLTISSL | |
| | QPEDFAVYFCQQGNTLPYTFGQGTKL | |
| | EIKGGGGSGGGGSGGGGSQVQLQESG | |
| | PGLVKPSETLSLTCTVSGVSLPDYGV | |
| | SWIRQPPGKGLEWIGVIWGSETTYYQ | |
| | SSLKSRVTISKDNSKNQVSLKLSSVT | |
| | AADTAVYYCAKHYYYGGSYAMDYWGQ | |
| | GTLVTVSSTTTPAPRPPTPAPTIASQ | |
| | PLSLRPEACRPAAGGAVHTRGLDFAC | |
| | DIYIWAPLAGTCGVLLLSLVITLYCK | |
| | RGRKKLLYIFKQPFMRPVQTTQEEDG | |
| | CSCRFPEEEEGGCELRVKFSRSADAP | |
| | AYQQGQNQLYNELNLGRREEYDVLDK | |
| | RRGRDPEMGGKPRRKNPQEGLYNELQ | |
| | KDKMAEAYSEIGMKGERRRGKGHDGL | |
| | YQGLSTATKDTYDALHMQALPPR | |
| | (SEQ ID NO: 225) | |
| Predicted | Anti-BCMA CAR arm: | |
| resultant | EVQLVESGGGLVQPGGSLRLSCAVSG | |
| proteins from | FALSNHGMSWVRRAPGKGLEWVSGIV | |
| duBCMA.4-P2A- | YSGSTYYAASVKGRFTISRDNSRNTL | |
| duCD19.1 | YLQMNSLRPEDTAIYYCSAHGGESDV | |
| | WGQGTTVTVSSASGGGGSGGGGSGGG | |
| | GSDIQLTQSPSSLSASVGDRVTITCR | |
| | ASQSISSYLNWYQQKPGKAPKLLIYA | |
| | ASSLQSGVPSRFSGSGSGTDFTLTIS | |
| | SLQPEDFATYYCQQSYSTPYTFGQGT | |
| | KVEIKTTTPAPRPPTPAPTIASQPLS | |
| | LRPEACRPAAGGAVHTRGLDFACDIY | |
| | IWAPLAGTCGVLLLSLVITLYCKRGR | |
| | KKLLYIFKQPFMRPVQTTQEEDGCSC | |
| | RFPEEEEGGCELRVKFSRSADAPAYQ | |
| | QGQNQLYNELNLGRREEYDVLDKRRG | |
| | RDPEMGGKPRRKNPQEGLYNELQKDK | |
| | MAEAYSEIGMKGERRRGKGHDGLYQG | |
| | LSTATKDTYDALHMQALPPRGSGATN | |
| | FSLLKQAGDVEENPG (SEQ ID | |
| | NO: 228) | |
| | Anti-CD19 CAR arm: | |
| | EIVMTQSPATLSLSPGERATLSCRAS | |
| | QDISKYLNWYQQKPGQAPRLLIYHTS | |
| | RLHSGIPARFSGSGSGTDYTLTISSL | |
| | QPEDFAVYFCQQGNTLPYTFGQGTKL | |
| | EIKGGGGSGGGGSGGGGSQVQLQESG | |
| | PGLVKPSETLSLTCTVSGVSLPDYGV | |
| | SWIRQPPGKGLEWIGVIWGSETTYYQ | |
| | SSLKSRVTISKDNSKNQVSLKLSSVT | |
| | AADTAVYYCAKHYYYGGSYAMDYWGQ | |
| | GTLVTVSSTTTPAPRPPTPAPTIASQ | |
| | PLSLRPEACRPAAGGAVHTRGLDFAC | |
| | DIYIWAPLAGTCGVLLLSLVITLYCK | |
| | RGRKKLLYIFKQPFMRPVQTTQEEDG | |
| | CSCRFPEEEEGGCELRVKFSRSADAP | |
| | AYQQGQNQLYNELNLGRREEYDVLDK | |
| | RRGRDPEMGGKPRRKNPQEGLYNELQ | |
| | KDKMAEAYSEIGMKGERRRGKGHDGL | |
| | YQGLSTATKDTYDALHMQALPPR | |
| | (SEQ ID NO: 225) | |
| Predicted | Anti-CD19 CAR arm: | |
| resultant | EIVMTQSPATLSLSPGERATLSCRAS | |
| proteins from | QDISKYLNWYQQKPGQAPRLLIYHTS | |
| duCD19.1-P2A- | RLHSGIPARFSGSGSGTDYTLTISSL | |
| duBCMA.4 | QPEDFAVYFCQQGNTLPYTFGQGTKL | |
| | EIKGGGGSGGGGSGGGGSQVQLQESG | |
| | PGLVKPSETLSLTCTVSGVSLPDYGV | |
| | SWIRQPPGKGLEWIGVIWGSETTYYQ | |
| | SSLKSRVTISKDNSKNQVSLKLSSVT | |
| | AADTAVYYCAKHYYYGGSYAMDYWGQ | |
| | GTLVTVSSTTTPAPRPPTPAPTIASQ | |
| | PLSLRPEACRPAAGGAVHTRGLDFAC | |
| | DIYIWAPLAGTCGVLLLSLVITLYCK | |
| | RGRKKLLYIFKQPFMRPVQTTQEEDG | |
| | CSCRFPEEEEGGCELRVKFSRSADAP | |
| | AYQQGQNQLYNELNLGRREEYDVLDK | |
| | RRGRDPEMGGKPRRKNPQEGLYNELQ | |
| | KDKMAEAYSEIGMKGERRRGKGHDGL | |
| | YQGLSTATKDTYDALHMQALPPRGSG | |
| | ATNFSLLKQAGDVEENPG (SEQ ID | |
| | NO: 229) | |
| | Anti-BCMA CAR arm: | |
| | EVQLVESGGGLVQPGGSLRLSCAVSG | |
| | FALSNHGMSWVRRAPGKGLEWVSGIV | |
| | YSGSTYYAASVKGRFTISRDNSRNTL | |
| | YLQMNSLRPEDTAIYYCSAHGGESDV | |
| | WGQGTTVTVSSASGGGGSGGGGSGGG | |
| | GSDIQLTQSPSSLSASVGDRVTITCR | |
| | ASQSISSYLNWYQQKPGKAPKLLIYA | |
| | ASSLQSGVPSRFSGSGSGTDETLTIS | |
| | SLQPEDFATYYCQQSYSTPYTFGQGT | |
| | KVEIKTTTPAPRPPTPAPTIASQPLS | |
| | LRPEACRPAAGGAVHTRGLDFACDIY | |
| | IWAPLAGTCGVLLLSLVITLYCKRGR | |
| | KKLLYIFKQPFMRPVQTTQEEDGCSC | |
| | RFPEEEEGGCELRVKFSRSADAPAYQ | |
| | QGQNQLYNELNLGRREEYDVLDKRRG | |
| | RDPEMGGKPRRKNPQEGLYNELQKDK | |
| | MAEAYSEIGMKGERRRGKGHDGLYQG | |
| | LSTATKDTYDALHMQALPPR (SEQ | |
| | ID NO: 230) | |

TABLE 22
Amino acid sequences of exemplary components of dual CARs

| SEQ | Name/ | |
|---|---|---|
| ID NO | Description | Sequence |
| PI61 | | |
| SEQ ID | HCDR1 | SYGMH |
| NO: 86 | (Kabat) | |
| SEQ ID | HCDR2 | VISYDGSNKYYADSVKG |
| NO: 87 | (Kabat) | |
| SEQ ID | HCDR3 | SGYALHDDYYGLDV |
| NO: 88 | (Kabat) | |
| SEQ ID | HCDR1 | GFTFSSY |
| NO: 47 | (Chothia) | |
| SEQ ID | HCDR2 | SYDGSN |
| NO: 89 | (Chothia) | |
| SEQ ID | HCDR3 | SGYALHDDYYGLDV |
| NO: 88 | (Chothia) | |
| SEQ ID | HCDR1 | GFTFSSYG |
| NO: 90 | (IMGT) | |
| SEQ ID | HCDR2 | ISYDGSNK |
| NO: 91 | (IMGT) | |
| SEQ ID | HCDR3 | GGSGYALHDDYYGLDV |
| NO: 92 | (IMGT) | |
| SEQ ID | VH | QVQLQESGGGVVQPGRSLRLSCAASGFTFSSYGMHWVRQAPGKGLEWV |
| NO: 93 | | AVISYDGSNKYYADSVKGRFTISRDNSKNTLYLQMNSLRAEDTAVYYC |
| | | GGSGYALHDDYYGLDVWGQGTLVTVSS |
| SEQ ID | DNA VH | CAAGTGCAGCTGCAGGAATCCGGTGGCGGAGTCGTGCAGCCTGGAAGG |
| NO: 94 | | AGCCTGAGACTCTCATGCGCCGCGTCAGGGTTCACCTTTTCCTCCTAC |
| | | GGGATGCATTGGGTCAGACAGGCCCCCGGAAAGGGACTCGAATGGGTG |
| | | GCTGTGATCAGCTACGACGGCTCCAACAAGTACTACGCCGACTCCGTG |
| | | AAAGGCCGGTTCACTATCTCCCGGGACAACTCCAAGAACACGCTGTAT |
| | | CTGCAAATGAATTCACTGCGCGCGGAGGATACCGCTGTGTACTACTGC |
| | | GGTGGCTCCGGTTACGCCCTGCACGATGACTATTACGGCCTTGACGTC |
| | | TGGGGCCAGGGAACCCTCGTGACTGTGTCCAGC |
| SEQ ID | LCDR1 | TGTSSDVGGYNYVS |
| NO: 95 | (Kabat) | |
| SEQ ID | LCDR2 | DVSNRPS |
| NO: 96 | (Kabat) | |
| SEQ ID | LCDR3 | SSYTSSSTLYV |
| NO: 97 | (Kabat) | |
| SEQ ID | LCDR1 | TSSDVGGYNY |
| NO: 98 | (Chothia) | |
| SEQ ID | LCDR2 | DVS |
| NO: 99 | (Chothia) | |
| SEQ ID | LCDR3 | YTSSSTLY |
| NO: 100 | (Chothia) | |
| SEQ ID | LCDR1 | SSDVGGYNY |
| NO: 101 | (IMGT) | |
| SEQ ID | LCDR2 | DVS |
| NO: 99 | (IMGT) | |
| SEQ ID | LCDR3 | SSYTSSSTLYV |
| NO: 97 | (IMGT) | |
| SEQ ID | VL | QSALTQPASVSGSPGQSITISCTGTSSDVGGYNYVSWYQQHPGKAPKL |
| NO: 102 | | MIYDVSNRPSGVSNRFSGSKSGNTASLTISGLQAEDEADYYCSSYTSS |
| | | STLYVFGSGTKVTVL |
| SEQ ID | DNA VL | CAGAGCGCACTGACTCAGCCGGCATCCGTGTCCGGTAGCCCCGGACAG |
| NO: 103 | | TCGATTACCATCTCCTGTACCGGCACCTCCTCCGACGTGGGAGGGTAC |
| | | AACTACGTGTCGTGGTACCAGCAGCACCCAGGAAAGGCCCCTAAGTTG |
| | | ATGATCTACGATGTGTCAAACCGCCCGTCTGGAGTCTCCAACCGGTTC |
| | | TCCGGCTCCAAGTCCGGCAACACCGCCAGCCTGACCATTAGCGGGCTG |
| | | CAAGCCGAGGATGAGGCCGACTACTACTGCTCGAGCTACACATCCTCG |
| | | AGCACCCTCTACGTGTTCGGCTCGGGGACTAAGGTCACCGTGCTG |
| SEQ ID | Linker | GGGGSGGGGSGGGGS |
| NO: 104 | | |
| SEQ ID | scFv (VH- | QVQLQESGGGVVQPGRSLRLSCAASGFTFSSYGMHWVRQAPGKGLEWV |
| NO: 105 | linker-VL) | AVISYDGSNKYYADSVKGRFTISRDNSKNTLYLQMNSLRAEDTAVYYC |
| | | GGSGYALHDDYYGLDVWGQGTLVTVSSGGGGSGGGGSGGGGSQSALTQ |
| | | PASVSGSPGQSITISCTGTSSDVGGYNYVSWYQQHPGKAPKLMIYDVS |
| | | NRPSGVSNRFSGSKSGNTASLTISGLQAEDEADYYCSSYTSSSTLYVF |
| | | GSGTKVTVL |
| SEQ ID | DNA scFv | CAAGTGCAGCTGCAGGAATCCGGTGGCGGAGTCGTGCAGCCTGGAAGG |
| NO: 106 | | AGCCTGAGACTCTCATGCGCCGCGTCAGGGTTCACCTTTTCCTCCTAC |
| | | GGGATGCATTGGGTCAGACAGGCCCCCGGAAAGGGACTCGAATGGGTG |
| | | GCTGTGATCAGCTACGACGGCTCCAACAAGTACTACGCCGACTCCGTG |
| | | AAAGGCCGGTTCACTATCTCCCGGGACAACTCCAAGAACACGCTGTAT |
| | | CTGCAAATGAATTCACTGCGCGCGGAGGATACCGCTGTGTACTACTGC |
| | | GGTGGCTCCGGTTACGCCCTGCACGATGACTATTACGGCCTTGACGTC |
| | | TGGGGCCAGGGAACCCTCGTGACTGTGTCCAGCGGTGGAGGAGGTTCG |
| | | GGCGGAGGAGGATCAGGAGGGGGTGGATCGCAGAGCGCACTGACTCAG |
| | | CCGGCATCCGTGTCCGGTAGCCCCGGACAGTCGATTACCATCTCCTGT |
| | | ACCGGCACCTCCTCCGACGTGGGAGGGTACAACTACGTGTCGTGGTAC |
| | | CAGCAGCACCCAGGAAAGGCCCCTAAGTTGATGATCTACGATGTGTCA |
| | | AACCGCCCGTCTGGAGTCTCCAACCGGTTCTCCGGCTCCAAGTCCGGC |
| | | AACACCGCCAGCCTGACCATTAGCGGGCTGCAAGCCGAGGATGAGGCC |
| | | GACTACTACTGCTCGAGCTACACATCCTCGAGCACCCTCTACGTGTTC |
| | | GGCTCGGGGACTAAGGTCACCGTGCTG |
| R1B6 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWVPYDVSWYFDY |
| NO: 46 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWVPYDVSWYFDY |
| NO: 46 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWVPYDVSWYFDY |
| NO: 51 | (IMGT) | |
| SEQ ID | VH | EVQLLESGGGLVQPGGSLRLSCAASGFTFSSYAMSWVRQAPGKGLEWV |
| NO: 52 | | SAISGSGGSTYYADSVKGRFTISRDNSKNTLYLQMNSLRAEDTAVYYC |
| | | ARREWVPYDVSWYFDYWGQGTLVTVSS |
| SEQ ID | DNA VH | GAAGTGCAGTTGCTGGAGTCAGGCGGAGGACTGGTGCAGCCCGGAGGA |
| NO: 53 | | TCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGTG |
| | | TCCGCCATTAGCGGTTCCGGCGGAAGCACTTACTATGCCGACTCTGTG |
| | | AAGGGCCGCTTCACTATCTCCCGGGACAACTCCAAGAACACCCTGTAT |
| | | CTCCAAATGAATTCCCTGAGGGCCGAAGATACCGCGGTGTACTACTGC |
| | | GCTAGACGGGAGTGGGTGCCCTACGATGTCAGCTGGTACTTCGACTAC |
| | | TGGGGACAGGGCACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKLLI |
| NO: 61 | | YAASSLQSGVPSRFSGSGSGTDFTLTISSLQPEDFATYYCQQSYSTPL |
| | | TFGQGTKVEIK |
| SEQ ID | DNA VL | GACATTCAAATGACTCAGTCCCCGTCCTCCCTCTCCGCCTCCGTGGGA |
| NO: 62 | | GATCGCGTCACGATCACGTGCAGGGCCAGCCAGAGCATCTCCAGCTAC |
| | | CTGAACTGGTACCAGCAGAAGCCAGGGAAGGCACCGAAGCTCCTGATC |
| | | TACGCCGCTAGCTCGCTGCAGTCCGGCGTCCCTTCACGGTTCTCGGGA |
| | | TCGGGCTCAGGCACCGACTTCACCCTGACCATTAGCAGCCTGCAGCCG |
| | | GAGGACTTCGCGACATACTACTGTCAGCAGTCATACTCCACCCCTCTG |
| | | ACCTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGLEWV |
| NO: 64 | linker-VL) | SAISGSGGSTYYADSVKGRFTISRDNSKNTLYLQMNSLRAEDTAVYYC |
| | | ARREWVPYDVSWYFDYWGQGTLVTVSSGGGGSGGGGSGGGGSGGGGSD |
| | | IQMTQSPSSLSASVGDRVTITCRASQSISSYLNWYQQKPGKAPKLLIY |
| | | AASSLQSGVPSRFSGSGSGTDFTLTISSLQPEDFATYYCQQSYSTPLT |
| | | FGQGTKVEIK |
| SEQ ID | DNA scFv | GAAGTGCAGTTGCTGGAGTCAGGCGGAGGACTGGTGCAGCCCGGAGGA |
| NO: 65 | | TCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGTG |
| | | TCCGCCATTAGCGGTTCCGGCGGAAGCACTTACTATGCCGACTCTGTG |
| | | AAGGGCCGCTTCACTATCTCCCGGGACAACTCCAAGAACACCCTGTAT |
| | | CTCCAAATGAATTCCCTGAGGGCCGAAGATACCGCGGTGTACTACTGC |
| | | GCTAGACGGGAGTGGGTGCCCTACGATGTCAGCTGGTACTTCGACTAC |
| | | TGGGGACAGGGCACTCTCGTGACTGTGTCCTCCGGTGGTGGTGGATCG |
| | | GGGGGTGGTGGTTCGGGCGGAGGAGGATCTGGAGGAGGAGGGTCGGAC |
| | | ATTCAAATGACTCAGTCCCCGTCCTCCCTCTCCGCCTCCGTGGGAGAT |
| | | CGCGTCACGATCACGTGCAGGGCCAGCCAGAGCATCTCCAGCTACCTG |
| | | AACTGGTACCAGCAGAAGCCAGGGAAGGCACCGAAGCTCCTGATCTAC |
| | | GCCGCTAGCTCGCTGCAGTCCGGCGTCCCTTCACGGTTCTCGGGATCG |
| | | GGCTCAGGCACCGACTTCACCCTGACCATTAGCAGCCTGCAGCCGGAG |
| | | GACTTCGCGACATACTACTGTCAGCAGTCATACTCCACCCCTCTGACC |
| | | TTCGGCCAAGGGACCAAAGTGGAGATCAAG |
| R1G5 | | |
| SEQ ID | HCDR1 | SYAMS |
| NO: 44 | (Kabat) | |
| SEQ ID | HCDR2 | AISGSGGSTYYADSVKG |
| NO: 45 | (Kabat) | |
| SEQ ID | HCDR3 | REWWGESWLFDY |
| NO: 76 | (Kabat) | |
| SEQ ID | HCDR1 | GFTFSSY |
| NO: 47 | (Chothia) | |
| SEQ ID | HCDR2 | SGSGGS |
| NO: 48 | (Chothia) | |
| SEQ ID | HCDR3 | REWWGESWLFDY |
| NO: 76 | (Chothia) | |
| SEQ ID | HCDR1 | GFTFSSYA |
| NO: 49 | (IMGT) | |
| SEQ ID | HCDR2 | ISGSGGST |
| NO: 50 | (IMGT) | |
| SEQ ID | HCDR3 | ARREWWGESWLFDY |
| NO: 77 | (IMGT) | |
| SEQ ID | VH | EVQLLESGGGLVQPGGSLRLSCAASGFTFSSYAMSWVRQAPGKGLEWV |
| NO: 78 | | SAISGSGGSTYYADSVKGRFTISRDNSKNTLYLQMNSLRAEDTAVYYC |
| | | ARREWWGESWLFDYWGQGTLVTVSS |
| SEQ ID | DNA VH | GAAGTGCAGTTGCTGGAGTCAGGCGGAGGACTGGTGCAGCCCGGAGGA |
| NO: 79 | | TCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGTG |
| | | TCCGCCATTAGCGGTTCCGGCGGAAGCACTTACTATGCCGACTCTGTG |
| | | AAGGGCCGCTTCACTATCTCCCGGGACAACTCCAAGAACACCCTGTAT |
| | | CTCCAAATGAATTCCCTGAGGGCCGAAGATACCGCGGTGTACTACTGC |
| | | GCTAGACGGGAGTGGTGGGGAGAAAGCTGGCTGTTCGACTACTGGGGA |
| | | CAGGGCACTCTCGTGACTGTGTCCTCC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPLT |
| NO: 56 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPL |
| NO: 59 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPLT |
| NO: 56 | (IMGT) | |
| SEQ ID | VL | DIQMTQSPSSLSASVGDRVTITCRASQSISSYLNWYQQKPGKAPKLLI |
| NO: 61 | | YAASSLQSGVPSRFSGSGSGTDFTLTISSLQPEDFATYYCQQSYSTPL |
| | | TFGQGTKVEIK |
| SEQ ID | DNA VL | GACATTCAAATGACTCAGTCCCCGTCCTCCCTCTCCGCCTCCGTGGGA |
| NO: 62 | | GATCGCGTCACGATCACGTGCAGGGCCAGCCAGAGCATCTCCAGCTAC |
| | | CTGAACTGGTACCAGCAGAAGCCAGGGAAGGCACCGAAGCTCCTGATC |
| | | TACGCCGCTAGCTCGCTGCAGTCCGGCGTCCCTTCACGGTTCTCGGGA |
| | | TCGGGCTCAGGCACCGACTTCACCCTGACCATTAGCAGCCTGCAGCCG |
| | | GAGGACTTCGCGACATACTACTGTCAGCAGTCATACTCCACCCCTCTG |
| | | ACCTTCGGCCAAGGGACCAAAGTGGAGATCAAG |
| SEQ ID | Linker | GGGGSGGGGSGGGGSGGGGS |
| NO: 63 | | |
| SEQ ID | scFv (VH- | EVQLLESGGGLVQPGGSLRLSCAASGFTFSSYAMSWVRQAPGKGLEWV |
| NO: 80 | linker-VL) | SAISGSGGSTYYADSVKGRFTISRDNSKNTLYLQMNSLRAEDTAVYYC |
| | | ARREWWGESWLFDYWGQGTLVTVSSGGGGSGGGGSGGGGSGGGGSDIQ |
| | | MTQSPSSLSASVGDRVTITCRASQSISSYLNWYQQKPGKAPKLLIYAA |
| | | SSLQSGVPSRFSGSGSGTDFTLTISSLQPEDFATYYCQQSYSTPLTFG |
| | | QGTKVEIK |
| SEQ ID | DNA scFv | GAAGTGCAGTTGCTGGAGTCAGGCGGAGGACTGGTGCAGCCCGGAGGA |
| NO: 81 | | TCGCTTCGCTTGAGCTGCGCAGCCTCAGGCTTTACCTTCTCCTCCTAC |
| | | GCCATGTCCTGGGTCAGACAGGCTCCCGGGAAGGGACTGGAATGGGTG |
| | | TCCGCCATTAGCGGTTCCGGCGGAAGCACTTACTATGCCGACTCTGTG |
| | | AAGGGCCGCTTCACTATCTCCCGGGACAACTCCAAGAACACCCTGTAT |
| | | CTCCAAATGAATTCCCTGAGGGCCGAAGATACCGCGGTGTACTACTGC |
| | | GCTAGACGGGAGTGGTGGGGAGAAAGCTGGCTGTTCGACTACTGGGGA |
| | | CAGGGCACTCTCGTGACTGTGTCCTCCGGTGGTGGTGGATCGGGGGGT |
| | | GGTGGTTCGGGCGGAGGAGGATCTGGAGGAGGAGGGTCGGACATTCAA |
| | | ATGACTCAGTCCCCGTCCTCCCTCTCCGCCTCCGTGGGAGATCGCGTC |
| | | ACGATCACGTGCAGGGCCAGCCAGAGCATCTCCAGCTACCTGAACTGG |
| | | TACCAGCAGAAGCCAGGGAAGGCACCGAAGCTCCTGATCTACGCCGCT |
| | | AGCTCGCTGCAGTCCGGCGTCCCTTCACGGTTCTCGGGATCGGGCTCA |
| | | GGCACCGACTTCACCCTGACCATTAGCAGCCTGCAGCCGGAGGACTTC |
| | | GCGACATACTACTGTCAGCAGTCATACTCCACCCCTCTGACCTTCGGC |
| | | CAAGGGACCAAAGTGGAGATCAAG |
| duBCMA.4 | | |
| SEQ ID | HCDR1 | NHGMS |
| NO: 231 | (Kabat) | |
| SEQ ID | HCDR2 | GIVYSGSTYYAASVKG |
| NO: 232 | (Kabat) | |
| SEQ ID | HCDR3 | HGGESDV |
| NO: 233 | (Kabat) | |
| SEQ ID | HCDR1 | GFALSNH |
| NO: 234 | (Chothia) | |
| SEQ ID | HCDR2 | VYSGS |
| NO: 235 | (Chothia) | |
| SEQ ID | HCDR3 | HGGESDV |
| NO: 233 | (Chothia) | |
| SEQ ID | HCDR1 | GFALSNHG |
| NO: 236 | (IMGT) | |
| SEQ ID | HCDR2 | IVYSGST |
| NO: 237 | (IMGT) | |
| SEQ ID | HCDR3 | SAHGGESDV |
| NO: 238 | (IMGT) | |
| SEQ ID | VH | EVQLVESGGGLVQPGGSLRLSCAVSGFALSNHGMSWVRRAPGKGLEWV |
| NO: 239 | | SGIVYSGSTYYAASVKGRFTISRDNSRNTLYLQMNSLRPEDTAIYYCS |
| | | AHGGESDVWGQGTTVTVSS |
| SEQ ID | DNA VH | GAAGTGCAATTGGTGGAATCAGGGGGAGGACTTGTGCAGCCTGGAGGA |
| NO: 262 | | TCGCTGAGACTGTCATGTGCCGTGTCCGGCTTTGCCCTGTCCAACCAC |
| | | GGGATGTCCTGGGTCCGCCGCGCGCCTGGAAAGGGCCTCGAATGGGTG |
| | | TCGGGTATTGTGTACAGCGGTAGCACCTACTATGCCGCATCCGTGAAG |
| | | GGGAGATTCACCATCAGCCGGGACAACTCCAGGAACACTCTGTACCTC |
| | | CAAATGAATTCGCTGAGGCCAGAGGACACTGCCATCTACTACTGCTCC |
| | | GCGCATGGCGGAGAGTCCGACGTCTGGGGACAGGGGACCACCGTGACC |
| | | GTGTCTAGC |
| SEQ ID | LCDR1 | RASQSISSYLN |
| NO: 54 | (Kabat) | |
| SEQ ID | LCDR2 | AASSLQS |
| NO: 55 | (Kabat) | |
| SEQ ID | LCDR3 | QQSYSTPYT |
| NO: 240 | (Kabat) | |
| SEQ ID | LCDR1 | SQSISSY |
| NO: 57 | (Chothia) | |
| SEQ ID | LCDR2 | AAS |
| NO: 58 | (Chothia) | |
| SEQ ID | LCDR3 | SYSTPY |
| NO: 241 | (Chothia) | |
| SEQ ID | LCDR1 | QSISSY |
| NO: 60 | (IMGT) | |
| SEQ ID | LCDR2 | AAS |
| NO: 58 | (IMGT) | |
| SEQ ID | LCDR3 | QQSYSTPYT |
| NO: 240 | (IMGT) | |
| SEQ ID | VL | DIQLTQSPSSLSASVGDRVTITCRASQSISSYLNWYQQKPGKAPKLLI |
| NO: 242 | | YAASSLQSGVPSRFSGSGSGTDFTLTISSLQPEDFATYYCQQSYSTPY |
| | | TFGQGTKVEIK |
| SEQ ID | DNA VL | GACATCCAGCTCACCCAGTCCCCGAGCTCGCTGTCCGCCTCCGTGGGA |
| NO: 263 | | GATCGGGTCACCATCACGTGCCGCGCCAGCCAGTCGATTTCCTCCTAC |
| | | CTGAACTGGTACCAACAGAAGCCCGGAAAAGCCCCGAAGCTTCTCATC |
| | | TACGCCGCCTCGAGCCTGCAGTCAGGAGTGCCCTCACGGTTCTCCGGC |
| | | TCCGGTTCCGGTACTGATTTCACCCTGACCATTTCCTCCCTGCAACCG |
| | | GAGGACTTCGCTACTTACTACTGCCAGCAGTCGTACTCCACCCCCTAC |
| | | ACTTTCGGACAAGGCACCAAGGTCGAAATCAAG |
| SEQ ID | Linker | ASGGGGSGGGGSGGGGS |
| NO: 243 | | |
| SEQ ID | scFv (VH- | EVQLVESGGGLVQPGGSLRLSCAVSGFALSNHGMSWVRRAPGKGLEWV |
| NO: 200 | linker-VL) | SGIVYSGSTYYAASVKGRFTISRDNSRNTLYLQMNSLRPEDTAIYYCS |
| | | AHGGESDVWGQGTTVTVSSASGGGGSGGGGSGGGGSDIQLTQSPSSLS |
| | | ASVGDRVTITCRASQSISSYLNWYQQKPGKAPKLLIYAASSLQSGVPS |
| | | RFSGSGSGTDFTLTISSLQPEDFATYYCQQSYSTPYTFGQGTKVEIK |
| SEQ ID | DNA scFv | GAAGTGCAATTGGTGGAATCAGGGGGAGGACTTGTGCAGCCTGGAGGA |
| NO: 201 | | TCGCTGAGACTGTCATGTGCCGTGTCCGGCTTTGCCCTGTCCAACCAC |
| | | GGGATGTCCTGGGTCCGCCGCGCGCCTGGAAAGGGCCTCGAATGGGTG |
| | | TCGGGTATTGTGTACAGCGGTAGCACCTACTATGCCGCATCCGTGAAG |
| | | GGGAGATTCACCATCAGCCGGGACAACTCCAGGAACACTCTGTACCTC |
| | | CAAATGAATTCGCTGAGGCCAGAGGACACTGCCATCTACTACTGCTCC |
| | | GCGCATGGCGGAGAGTCCGACGTCTGGGGACAGGGGACCACCGTGACC |
| | | GTGTCTAGCGCGTCCGGCGGAGGCGGCAGCGGGGGTGGTGGTTCAGGG |
| | | GGCGGCGGATCGGACATCCAGCTCACCCAGTCCCCGAGCTCGCTGTCC |
| | | GCCTCCGTGGGAGATCGGGTCACCATCACGTGCCGCGCCAGCCAGTCG |
| | | ATTTCCTCCTACCTGAACTGGTACCAACAGAAGCCCGGAAAAGCCCCG |
| | | AAGCTTCTCATCTACGCCGCCTCGAGCCTGCAGTCAGGAGTGCCCTCA |
| | | CGGTTCTCCGGCTCCGGTTCCGGTACTGATTTCACCCTGACCATTTCC |
| | | TCCCTGCAACCGGAGGACTTCGCTACTTACTACTGCCAGCAGTCGTAC |
| | | TCCACCCCCTACACTTTCGGACAAGGCACCAAGGTCGAAATCAAG |
| duCD19.1 | | |
| SEQ ID | HCDR1 | GVSLPDYGVS |
| NO: 244 | | |
| SEQ ID | HCDR2 | VIWGSETTYYQSSLKS |
| NO: 245 | | |
| SEQ ID | HCDR3 | HYYYGGSYAMDY |
| NO: 246 | | |
| SEQ ID | HCDR1 | DYGVS |
| NO: 295 | (Kabat) | |
| SEQ ID | HCDR2 | VIWGSETTYYQSSLKS |
| NO: 245 | (Kabat) | |
| SEQ ID | HCDR3 | HYYYGGSYAMDY |
| NO: 246 | (Kabat) | |
| SEQ ID | HCDR1 | GVSLPDY |
| NO: 310 | (Chothia) | |
| SEQ ID | HCDR2 | WGSET |
| NO: 311 | (Chothia) | |
| SEQ ID | HCDR3 | HYYYGGSYAMDY |
| NO: 246 | (Chothia) | |
| SEQ ID | HCDR1 | GVSLPDYG |
| NO: 312 | (IMGT) | |
| SEQ ID | HCDR2 | IWGSETT |
| NO: 313 | (IMGT) | |
| SEQ ID | HCDR3 | AKHYYYGGSYAMDY |
| NO: 314 | (IMGT) | |
| SEQ ID | VH | QVQLQESGPGLVKPSETLSLTCTVSGVSLPDYGVSWIRQPPGKGLEWI |
| NO: 250 | | GVIWGSETTYYQSSLKSRVTISKDNSKNQVSLKLSSVTAADTAVYYCA |
| | | KHYYYGGSYAMDYWGQGTLVTVSS |
| SEQ ID | DNA VH | CAGGTCCAACTCCAAGAAAGCGGACCGGGTCTTGTGAAGCCATCAGAA |
| NO: 315 | | ACTCTTTCACTGACTTGTACTGTGAGCGGAGTGTCTCTCCCCGATTAC |
| | | GGGGTGTCTTGGATCAGACAGCCACCGGGGAAGGGTCTGGAATGGATT |
| | | GGAGTGATTTGGGGCTCTGAGACTACTTACTACCAATCATCCCTCAAG |
| | | TCACGCGTCACCATCTCAAAGGACAACTCTAAGAATCAGGTGTCACTG |
| | | AAACTGTCATCTGTGACCGCAGCCGACACCGCCGTGTACTATTGCGCT |
| | | AAGCATTACTATTATGGCGGGAGCTACGCAATGGATTACTGGGGACAG |
| | | GGTACTCTGGTCACCGTGTCCAGC |
| SEQ ID | LCDR1 | RASQDISKYLN |
| NO: 247 | | |
| SEQ ID | LCDR2 | HTSRLHS |
| NO: 248 | | |
| SEQ ID | LCDR3 | QQGNTLPYT |
| NO: 249 | | |
| SEQ ID | LCDR1 | RASQDISKYLN |
| NO: 247 | (Kabat) | |
| SEQ ID | LCDR2 | HTSRLHS |
| NO: 248 | (Kabat) | |
| SEQ ID | LCDR3 | QQGNTLPYT |
| NO: 249 | (Kabat) | |
| SEQ ID | LCDR1 | SQDISKY |
| NO: 316 | (Chothia) | |
| SEQ ID | LCDR2 | HTS |
| NO: 317 | (Chothia) | |
| SEQ ID | LCDR3 | GNTLPY |
| NO: 318 | (Chothia) | |
| SEQ ID | LCDR1 | QDISKY |
| NO: 319 | (IMGT) | |
| SEQ ID | LCDR2 | HTS |
| NO: 317 | (IMGT) | |
| SEQ ID | LCDR3 | QQGNTLPYT |
| NO: 300 | (IMGT) | |
| SEQ ID | VL | EIVMTQSPATLSLSPGERATLSCRASQDISKYLNWYQQKPGQAPRLLI |
| NO: 251 | | YHTSRLHSGIPARFSGSGSGTDYTLTISSLQPEDFAVYFCQQGNTLPY |
| | | TFGQGTKLEIK |
| SEQ ID | DNA VL | GAAATTGTGATGACCCAGTCACCCGCCACTCTTAGCCTTTCACCCGGT |
| NO: 320 | | GAGCGCGCAACCCTGTCTTGCAGAGCCTCCCAAGACATCTCAAAATAC |
| | | CTTAATTGGTATCAACAGAAGCCCGGACAGGCTCCTCGCCTTCTGATC |
| | | TACCACACCAGCCGGCTCCATTCTGGAATCCCTGCCAGGTTCAGCGGT |
| | | AGCGGATCTGGGACCGACTACACCCTCACTATCAGCTCACTGCAGCCA |
| | | GAGGACTTCGCTGTCTATTTCTGTCAGCAAGGGAACACCCTGCCCTAC |
| | | ACCTTTGGACAGGGCACCAAGCTCGAGATTAAA |
| SEQ ID | Linker | GGGGSGGGGSGGGGS |
| NO: 104 | | |
| SEQ ID | CAR2 scFv | EIVMTQSPATLSLSPGERATLSCRASQDISKYLNWYQQKPGQAPRLLI |
| NO: 211 | domain-aa | YHTSRLHSGIPARFSGSGSGTDYTLTISSLQPEDFAVYFCQQGNTLPY |
| | (Linker is | TFGQGTKLEIKGGGGSGGGGSGGGGSQVQLQESGPGLVKPSETLSLTC |
| | underlined) | TVSGVSLPDYGVSWIRQPPGKGLEWIGVIWGSETTYYQSSLKSRVTIS |
| | | KDNSKNQVSLKLSSVTAADTAVYYCAKHYYYGGSYAMDYWGQGTLVTV |
| | | SS |
| SEQ ID | CAR2 scFv | GAAATTGTGATGACCCAGTCACCCGCCACTCTTAGCCTTTCACCCGGT |
| NO: 305 | domain-nt | GAGCGCGCAACCCTGTCTTGCAGAGCCTCCCAAGACATCTCAAAATAC |
| | | CTTAATTGGTATCAACAGAAGCCCGGACAGGCTCCTCGCCTTCTGATC |
| | | TACCACACCAGCCGGCTCCATTCTGGAATCCCTGCCAGGTTCAGCGGT |
| | | AGCGGATCTGGGACCGACTACACCCTCACTATCAGCTCACTGCAGCCA |
| | | GAGGACTTCGCTGTCTATTTCTGTCAGCAAGGGAACACCCTGCCCTAC |
| | | ACCTTTGGACAGGGCACCAAGCTCGAGATTAAAGGTGGAGGTGGCAGC |
| | | GGAGGAGGTGGGTCCGGCGGTGGAGGAAGCCAGGTCCAACTCCAAGAA |
| | | AGCGGACCGGGTCTTGTGAAGCCATCAGAAACTCTTTCACTGACTTGT |
| | | ACTGTGAGCGGAGTGTCTCTCCCCGATTACGGGGTGTCTTGGATCAGA |
| | | CAGCCACCGGGGAAGGGTCTGGAATGGATTGGAGTGATTTGGGGCTCT |
| | | GAGACTACTTACTACCAATCATCCCTCAAGTCACGCGTCACCATCTCA |
| | | AAGGACAACTCTAAGAATCAGGTGTCACTGAAACTGTCATCTGTGACC |
| | | GCAGCCGACACCGCCGTGTACTATTGCGCTAAGCATTACTATTATGGC |
| | | GGGAGCTACGCAATGGATTACTGGGGACAGGGTACTCTGGTCACCGTG |
| | | TCCAGC |
| SEQ ID | Full CAR | EIVMTQSPATLSLSPGERATLSCRASQDISKYLNWYQQKPGQAPRLLI |
| NO: 225 | | YHTSRLHSGIPARFSGSGSGTDYTLTISSLQPEDFAVYFCQQGNTLPY |
| | | TFGQGTKLEIKGGGGSGGGGSGGGGSQVQLQESGPGLVKPSETLSLTC |
| | | TVSGVSLPDYGVSWIRQPPGKGLEWIGVIWGSETTYYQSSLKSRVTIS |
| | | KDNSKNQVSLKLSSVTAADTAVYYCAKHYYYGGSYAMDYWGQGTLVTV |
| | | SSTTTPAPRPPTPAPTIASQPLSLRPEACRPAAGGAVHTRGLDFACDI |
| | | YIWAPLAGTCGVLLLSLVITLYCKRGRKKLLYIFKQPFMRPVQTTQEE |
| | | DGCSCRFPEEEEGGCELRVKFSRSADAPAYQQGQNQLYNELNLGRREE |
| | | YDVLDKRRGRDPEMGGKPRRKNPQEGLYNELQKDKMAEAYSEIGMKGE |
| | | RRRGKGHDGLYQGLSTATKDTYDALHMQALPPR |

In some embodiments, disclosed herein is an isolated nucleic acid molecule comprising a first nucleic acid sequence encoding a first CAR polypeptide and a second nucleic acid sequence encoding a second CAR polypeptide, wherein the first CAR polypeptide comprises a first antigen-binding domain which is an anti-BCMA binding domain (e.g., human anti-BCMA binding domain), a first transmembrane domain, and a first intracellular signaling domain, and wherein the second CAR polypeptide comprises a second antigen-binding domain which is an anti-CD19 binding domain, a second transmembrane domain, and a second intracellular signaling domain. In some embodiments, the first CAR polypeptide comprises a VH comprising a HC CDR1, HC CDR2, and HC CDR3 of an anti-BCMA sequence listed in Table 20 or 26 and a VL comprising a LC CDR1, LC CDR2, and LC CDR3 of an anti-BCMA sequence listed in Table 20 or 26, wherein the VH and VL are connected by a linker comprising the amino acid sequence of SEQ ID NO: 243. In some embodiment, the first CAR polypeptide comprises a VH and VL comprising the amino acid sequences of SEQ ID NOs: 239 and 242, respectively, wherein the VH and VL are connected by a linker comprising the amino acid sequence of SEQ ID NO: 243. In some embodiment, the first CAR polypeptide comprises an scFv comprising the amino acid sequence of SEQ ID NO: 200. In some embodiment, the first CAR polypeptide comprises the amino acid sequence of SEQ ID NO: 230 or 228. In some embodiments, the second CAR polypeptide comprises a HC CDR1, HC CDR2, HC CDR3, LC CDR1, LC CDR2, and/or LC CDR3 of an anti-CD19 sequence listed in Table 19 or Table 22 (e.g., a HC CDR1, HC CDR2, HC CDR3, LC CDR1, LC CDR2, and LC CDR3 comprising the amino acid sequences of SEQ ID NOs: 295 and 245-249, respectively). In some embodiments, the second CAR polypeptide comprises a VH and/or VL of an anti-CD19 sequence listed in Table 19 or Table 22 (e.g., a VH and VL comprising the amino acid sequences of SEQ ID NOs: 250 and 251, respectively), or an amino acid sequence having at least about 85%, 90%, 95%, or 99% sequence identity thereto. In some embodiments, the second CAR polypeptide comprises a scFv of an anti-CD19 sequence listed in Table 19 or Table 22 (e.g., a scFv comprising the amino acid sequence of SEQ ID NO: 211), or an amino acid sequence having at least about 85%, 90%, 95%, or 99% sequence identity thereto. In some embodiments, the second CAR polypeptide comprises a CAR polypeptide of an anti-CD19 sequence listed in Table 19 or Table 22 (e.g., a CAR polypeptide comprising the amino acid sequence of SEQ ID NO: 225 or 229), or an amino acid sequence having at least about 85%, 90%, 95%, or 99% sequence identity thereto. In some embodiments, the nucleic acid molecule comprises the nucleic acid sequence of SEQ ID NO: 221 or 223. In some embodiments, the nucleic acid molecule encodes the amino acid sequence of SEQ ID NO: 220 or 222, with or without the signal peptide of SEQ ID NO: 1.

### Multi-Specific CARs

In an embodiment, a CAR of the invention is a multi-specific CAR. In one embodiment, the multi-specific CAR is a bispecific CAR. In one embodiment, the bispecific CAR comprises an antigen binding domain which is a bispecific antibody molecule. A bispecific antibody has specificity for no more than two antigens. A bispecific antibody molecule is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment the first and second epitopes overlap. In an embodiment the first and second epitopes do not overlap. In an embodiment the first and second epitopes are on different antigens, e.g., different proteins (or different subunits of a multimeric protein). In an embodiment a bispecific antibody molecule comprises a heavy chain variable domain sequence and a light chain variable domain sequence which have binding specificity for a first epitope and a heavy chain variable domain sequence and a light chain variable domain sequence which have binding specificity for a second epitope. In an embodiment a bispecific antibody molecule comprises a half antibody having binding specificity for a first epitope and a half antibody having binding specificity for a second epitope. In an embodiment a bispecific antibody molecule comprises a half antibody, or fragment thereof, having binding specificity for a first epitope and a half antibody, or fragment thereof, having binding specificity for a second epitope. In an embodiment a bispecific antibody molecule comprises a scFv, or fragment thereof, have binding specificity for a first epitope and a scFv, or fragment thereof, have binding specificity for a second epitope.

In some embodiments, a CAR of the invention comprises an antigen binding domain that is a multi-specific (e.g., a bispecific or a trispecific) antibody molecule. Protocols for generating bispecific or heterodimeric antibody molecules are known in the art; including but not limited to, for example, the “knob in a hole” approach described in, e.g., U.S. Pat. No. 5,731,168; the electrostatic steering Fc pairing as described in, e.g., WO 09/089004, WO 06/106905 and WO 2010/129304; Strand Exchange Engineered Domains (SEED) heterodimer formation as described in, e.g., WO 07/110205; Fab arm exchange as described in, e.g., WO 08/119353, WO 2011/131746, and WO 2013/060867; double antibody conjugate, e.g., by antibody cross-linking to generate a bi-specific structure using a heterobifunctional reagent having an amine-reactive group and a sulfhydryl reactive group as described in, e.g., U.S. Pat. No. 4,433,059; bispecific antibody determinants generated by recombining half antibodies (heavy-light chain pairs or Fabs) from different antibodies through cycle of reduction and oxidation of disulfide bonds between the two heavy chains, as described in, e.g., U.S. Pat. No. 4,444,878; trifunctional antibodies, e.g., three Fab′ fragments cross-linked through sulfhydryl reactive groups, as described in, e.g., U.S. Pat. No. 5,273,743; biosynthetic binding proteins, e.g., pair of scFvs cross-linked through C-terminal tails preferably through disulfide or amine-reactive chemical cross-linking, as described in, e.g., U.S. Pat. No. 5,534,254; bifunctional antibodies, e.g., Fab fragments with different binding specificities dimerized through leucine zippers (e.g., c-fos and c-jun) that have replaced the constant domain, as described in, e.g., U.S. Pat. No. 5,582,996; bispecific and oligospecific mono- and oligovalent receptors, e.g., VH—CH1 regions of two antibodies (two Fab fragments) linked through a polypeptide spacer between the CH1 region of one antibody and the VH region of the other antibody typically with associated light chains, as described in, e.g., U.S. Pat. No. 5,591,828; bispecific DNA-antibody conjugates, e.g., crosslinking of antibodies or Fab fragments through a double stranded piece of DNA, as described in, e.g., U.S. Pat. No. 5,635,602; bispecific fusion proteins, e.g., an expression construct containing two scFvs with a hydrophilic helical peptide linker between them and a full constant region, as described in, e.g., U.S. Pat. No. 5,637,481; multivalent and multispecific binding proteins, e.g., dimer of polypeptides having first domain with binding region of Ig heavy chain variable region, and second domain with binding region of Ig light chain variable region, generally termed diabodies (higher order structures are also encompassed creating for bispecific, trispecific, or tetraspecific molecules, as described in, e.g., U.S. Pat. No. 5,837,242; minibody constructs with linked VL and VH chains further connected with peptide spacers to an antibody hinge region and CH3 region, which can be dimerized to form bispecific/multivalent molecules, as described in, e.g., U.S. Pat. No. 5,837,821; VH and VL domains linked with a short peptide linker (e.g., 5 or 10 amino acids) or no linker at all in either orientation, which can form dimers to form bispecific diabodies; trimers and tetramers, as described in, e.g., U.S. Pat. No. 5,844,094; String of VH domains (or VL domains in family members) connected by peptide linkages with crosslinkable groups at the C-terminus further associated with VL domains to form a series of FVs (or scFvs), as described in, e.g., U.S. Pat. No. 5,864,019; and single chain binding polypeptides with both a VH and a VL domain linked through a peptide linker are combined into multivalent structures through non-covalent or chemical crosslinking to form, e.g., homobivalent, heterobivalent, trivalent, and tetravalent structures using both scFV or diabody type format, as described in, e.g., U.S. Pat. No. 5,869,620. Additional exemplary multispecific and bispecific molecules and methods of making the same are found, for example, in U.S. Pat. Nos. 5,910,573, 5,932,448, 5,959,083, 5,989,830, 6,005,079, 6,239,259, 6,294,353, 6,333,396, 6,476,198, 6,511,663, 6,670,453, 6,743,896, 6,809,185, 6,833,441, 7,129,330, 7,183,076, 7,521,056, 7,527,787, 7,534,866, 7,612,181, US2002004587A1, US2002076406A1, US2002103345A1, US2003207346A1, US2003211078A1, US2004219643A1, US2004220388A1, US2004242847A1, US2005003403A1, US2005004352A1, US2005069552A1, US2005079170A1, US2005100543A1, US2005136049A1, US2005136051A1, US2005163782A1, US2005266425A1, US2006083747A1, US2006120960A1, US2006204493A1, US2006263367A1, US2007004909A1, US2007087381A1, US2007128150A1, US2007141049A1, US2007154901A1, US2007274985A1, US2008050370A1, US2008069820A1, US2008152645A1, US2008171855A1, US2008241884A1, US2008254512A1, US2008260738A1, US2009130106A1, US2009148905A1, US2009155275A1, US2009162359A1, US2009162360A1, US2009175851A1, US2009175867A1, US2009232811A1, US2009234105A1, US2009263392A1, US2009274649A1, EP346087A2, WO0006605A2, WO02072635A2, WO04081051A1, WO06020258A2, WO2007044887A2, WO2007095338A2, WO2007137760A2, WO2008119353A1, WO2009021754A2, WO2009068630A1, WO9103493A1, WO9323537A1, WO9409131A1, WO9412625A2, WO9509917A1, WO9637621A2, WO9964460A1. The contents of the above-referenced applications are incorporated herein by reference in their entireties.

Within each antibody or antibody fragment (e.g., scFv) of a bispecific antibody molecule, the VH can be upstream or downstream of the VL. In some embodiments, the upstream antibody or antibody fragment (e.g., scFv) is arranged with its VH (VH₁) upstream of its VL (VL₁) and the downstream antibody or antibody fragment (e.g., scFv) is arranged with its VL (VL₂) upstream of its VH (VH₂), such that the overall bispecific antibody molecule has the arrangement VH₁—VL₁-VL₂-VH₂. In other embodiments, the upstream antibody or antibody fragment (e.g., scFv) is arranged with its VL (VL₁) upstream of its VH (VH₁) and the downstream antibody or antibody fragment (e.g., scFv) is arranged with its VH (VH₂) upstream of its VL (VL₂), such that the overall bispecific antibody molecule has the arrangement VL₁-VH₁—VH₂—VL₂. Optionally, a linker is disposed between the two antibodies or antibody fragments (e.g., scFvs), e.g., between VL₁ and VL₂ if the construct is arranged as VH₁—VL₁-VL₂-VH₂, or between VH₁ and VH₂ if the construct is arranged as VL₁-VH₁—VH₂—VL₂. The linker may be a linker as described herein, e.g., a (Gly4-Ser)n linker, wherein n is 1, 2, 3, 4, 5, or 6, preferably 4 (SEQ ID NO: 26). In general, the linker between the two scFvs should be long enough to avoid mispairing between the domains of the two scFvs. Optionally, a linker is disposed between the VL and VH of the first scFv. Optionally, a linker is disposed between the VL and VH of the second scFv. In constructs that have multiple linkers, any two or more of the linkers can be the same or different. Accordingly, in some embodiments, a bispecific CAR comprises VLs, VHs, and optionally one or more linkers in an arrangement as described herein.

In one aspect, the bispecific antibody molecule is characterized by a first immunoglobulin variable domain sequence, e.g., a scFv, which has binding specificity for BCMA, e.g., comprises a scFv as described herein, or comprises the light chain CDRs and/or heavy chain CDRs from a BCMA scFv described herein, and a second immunoglobulin variable domain sequence that has binding specificity for a second epitope on a different antigen. In one aspect, the second immunoglobulin variable domain sequence has binding specificity for an antigen expressed on AML cells, e.g., an antigen other than BCMA. For example, the second immunoglobulin variable domain sequence has binding specificity for CD123. As another example, the second immunoglobulin variable domain sequence has binding specificity for CLL-1. As another example, the second immunoglobulin variable domain sequence has binding specificity for CD34. As another example, the second immunoglobulin variable domain sequence has binding specificity for FLT3. For example, the second immunoglobulin variable domain sequence has binding specificity for folate receptor beta. In some aspects, the second immunoglobulin variable domain sequence has binding specificity for an antigen expressed on B-cells, for example, CD10, CD19, CD20, CD22, CD34, CD123, FLT-3, ROR1, CD79b, CD179b, or CD79a.

### Diabody CAR

In some embodiments, a CAR of the invention is a bispecific CAR. In some embodiments, a CAR of the invention is a diabody CAR. In some embodiments, the diabody CAR comprises an antigen binding domain that binds to a first antigen and a second antigen. In some embodiments, the antigen binding domain comprises a VH1, a VL1, a VH2, and a VL2, wherein the VH1 and VL1 bind to the first antigen and the VH2 and VL2 bind to the second antigen. In some embodiments, the antigen binding domain has the arrangement VH1—optionally linker 1 (“L1”)—VH2—optionally linker 2 (“L2”)-VL2-optionally linker 3 (“L3”)-VL1 from the N-terminus to the C-terminus. In some embodiments, the antigen binding domain has the arrangement VH1—optionally L1-VL2-optionally L2-VH2—optionally L3-VL1 from the N-terminus to the C-terminus. In some embodiments, the antigen binding domain has the arrangement VL1-optionally L1-VH2—optionally L2-VL2—optionally L3-VH1 from the N-terminus to the C-terminus. In some embodiments, the antigen binding domain has the arrangement VL1-optionally L1-VL2-optionally L2-VH2—optionally L3-VH1 from the N-terminus to the C-terminus. In some embodiments, the antigen binding domain has the arrangement VH2—optionally L1-VH1—optionally L2-VL1-optionally L3-VL2 from the N-terminus to the C-terminus. In some embodiments, the antigen binding domain has the arrangement VH2—optionally L1-VL1-optionally L2-VH1—optionally L3-VL2 from the N-terminus to the C-terminus. In some embodiments, the antigen binding domain has the arrangement VL2-optionally L1-VH1—optionally L2-VL1-optionally L3-VH2 from the N-terminus to the C-terminus. In some embodiments, the antigen binding domain has the arrangement VL2-optionally L1-VL1—optionally L2-VH1—optionally L3-VH2 from the N-terminus to the C-terminus. In some embodiments, the antigen binding domain has the arrangement VH1—linker 1 (“L1”)—VH2—linker 2 (“L2”)-VL2-linker 3 (“L3”)-VL1 from the N-terminus to the C-terminus. In some embodiments, the antigen binding domain has the arrangement VH1-L1-VL2-L2-VH2-L3-VL1 from the N-terminus to the C-terminus. In some embodiments, the antigen binding domain has the arrangement VL1-L1-VH2-L2-VL2-L3-VH1 from the N-terminus to the C-terminus. In some embodiments, the antigen binding domain has the arrangement VL1-L1-VL2-L2-VH2-L3-VH1 from the N-terminus to the C-terminus. In some embodiments, the antigen binding domain has the arrangement VH2-L1-VH1-L2-VL1-L3-VL2 from the N-terminus to the C-terminus. In some embodiments, the antigen binding domain has the arrangement VH2-L1-VL1-L2-VH1-L3-VL2 from the N-terminus to the C-terminus. In some embodiments, the antigen binding domain has the arrangement VL2-L1-VH1-L2-VL1-L3-VH2 from the N-terminus to the C-terminus. In some embodiments, the antigen binding domain has the arrangement VL2-L1-VL1-L2-VH1-L3—VH2 from the N-terminus to the C-terminus. In some embodiments, the variable regions are fused by a linker comprising the amino acid sequence of GGGGSGGGGS (SEQ ID NO: 5). In some embodiments, the variable regions are fused by a linker comprising the amino acid sequence of GGGGSGGGGSGGGGSGGGGS (SEQ ID NO: 63). In some embodiments, L1 comprises the amino acid sequence of SEQ ID NO: 5. In some embodiments, L2 comprises the amino acid sequence of SEQ ID NO: 63. In some embodiments, L3 comprises the amino acid sequence of SEQ ID NO: 5. In some embodiments, the VH1, VL1, VH2, or VL2 comprises a CDR, a VH, or a VL sequence disclosed herein, or an amino acid sequence having at least about 85%, 90%, 95%, or 99% sequence identity thereto. In some embodiments, a diabody disclosed herein comprises an engineered disulfide bridge, e.g., to stabilize the diabody and/or to facilitate correct pairing of the VH and VL. In some embodiments, the engineered disulfide bridge is between the variable region that is most proximal to the hinge region (e.g., the VH or VL region that is most proximal to the hinge region) and its corresponding pairing partner (e.g., the corresponding VL or the corresponding VH).

In some embodiments, the first antigen and the second antigen are different. In some embodiments, the first or second antigen is chosen from an antigen expressed on B cells, an antigen expressed on acute myeloid leukemia cells, or an antigen on solid tumor cells. In some embodiments, the first or second antigen is chosen from CD10, CD19, CD20, CD22, CD34, CD123, BCMA, FLT-3, ROR1, CD79b, CD179b, CD79a, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

In some embodiments, the first antigen is BCMA and the second antigen is CD19. In some embodiments, the CAR comprises an antigen binding domain that binds to BCMA and CD19. In some embodiments, the antigen binding domain comprises a VH₁ and a VL₁ that bind to BCMA (“BCMA VH” and “BCMA VL”) and a VH₂ and a VL₂ that bind to CD19 (“CD19 VH” and “CD19 VL”). In some embodiments, the antigen binding domain has the arrangement BCMA VH—optionally linker 1 (“L1”)—CD19 VH—optionally linker 2 (“L2”)-CD19 VL—optionally linker 3 (“L3”)-BCMA VL from the N-terminus to the C-terminus. In some embodiments, the antigen binding domain has the arrangement BCMA VH—optionally L1-CD19 VL—optionally L2-CD19 VH—optionally L3-BCMA VL from the N-terminus to the C-terminus. In some embodiments, the antigen binding domain has the arrangement BCMA VL—optionally L1-CD19 VH—optionally L2-CD19 VL—optionally L3-BCMA VH from the N-terminus to the C-terminus. In some embodiments, the antigen binding domain has the arrangement BCMA VL—optionally L1-CD19 VL—optionally L2-CD19 VH—optionally L3-BCMA VH from the N-terminus to the C-terminus. In some embodiments, the antigen binding domain has the arrangement CD19 VH—optionally L1-BCMA VH—optionally L2-BCMA VL—optionally L3-CD19 VL from the N-terminus to the C-terminus. In some embodiments, the antigen binding domain has the arrangement CD19 VH—optionally L1-BCMA VL—optionally L2-BCMA VH—optionally L3-CD19 VL from the N-terminus to the C-terminus. In some embodiments, the antigen binding domain has the arrangement CD19 VL—optionally L1-BCMA VH—optionally L2-BCMA VL—optionally L3-CD19 VH from the N-terminus to the C-terminus. In some embodiments, the antigen binding domain has the arrangement CD19 VL—optionally L1-BCMA VL—optionally L2-BCMA VH—optionally L3-CD19 VH from the N-terminus to the C-terminus. In some embodiments, the antigen binding domain has the arrangement BCMA VH—linker 1 (“L1”)-CD19 VH—linker 2 (“L2”)-CD19 VL—linker 3 (“L3”)-BCMA VL from the N-terminus to the C-terminus. In some embodiments, the antigen binding domain has the arrangement BCMA VH—L1-CD19 VL—L2-CD19 VH—L3-BCMA VL from the N-terminus to the C-terminus. In some embodiments, the antigen binding domain has the arrangement BCMA VL—L1-CD19 VH—L2-CD19 VL—L3-BCMA VH from the N-terminus to the C-terminus. In some embodiments, the antigen binding domain has the arrangement BCMA VL—L1-CD19 VL—L2-CD19 VH—L3-BCMA VH from the N-terminus to the C-terminus. In some embodiments, the antigen binding domain has the arrangement CD19 VH—L1-BCMA VH—L2-BCMA VL—L3-CD19 VL from the N-terminus to the C-terminus. In some embodiments, the antigen binding domain has the arrangement CD19 VH—L1-BCMA VL—L2-BCMA VH—L3-CD19 VL from the N-terminus to the C-terminus. In some embodiments, the antigen binding domain has the arrangement CD19 VL—L1-BCMA VH—L2-BCMA VL—L3-CD19 VH from the N-terminus to the C-terminus. In some embodiments, the antigen binding domain has the arrangement CD19 VL—L1-BCMA VL—L2-BCMA VH—L3-CD19 VH from the N-terminus to the C-terminus. In some embodiments, the variable regions are fused by a linker comprising the amino acid sequence of SEQ ID NO: 5 or 63, or an amino acid sequence having at least 80, 85, 90, 95, or 99% identity thereto. In some embodiments, L1 comprises the amino acid sequence of SEQ ID NO: 5. In some embodiments, L2 comprises the amino acid sequence of SEQ ID NO: 63. In some embodiments, L3 comprises the amino acid sequence of SEQ ID NO: 5.

In some embodiments, the BCMA VH comprises CDR or VH sequences disclosed herein, e.g., CDR or VH sequences disclosed in Tables 3-15, 19, 20, 22, 26, and 31, or an amino acid sequence having at least 80, 85, 90, 95, or 99% identity thereto. In some embodiments, the BCMA VL comprises CDR or VL sequences disclosed herein, e.g., CDR or VL sequences disclosed in Tables 3-15, 19, 20, 22, 26, and 31, or an amino acid sequence having at least 80, 85, 90, 95, or 99% identity thereto. In some embodiments, the CD19 VH comprises CDR or VH sequences disclosed herein, e.g., CDR or VH sequences disclosed in Tables 2, 19, 22, and 31, or an amino acid sequence having at least 80, 85, 90, 95, or 99% identity thereto. In some embodiments, the CD19 VL comprises CDR or VL sequences disclosed herein, e.g., CDR or VL sequences disclosed in Tables 2, 19, 22, and 31, or an amino acid sequence having at least 80, 85, 90, 95, or 99% identity thereto.

In some embodiments, the CAR, e.g., a diabody CAR, further comprises a hinge region, a transmembrane domain, and/or an intracellular signaling domain. In some embodiments, the hinge region comprises a CD8 hinge region. In some embodiments, the hinge region comprises a hinge region sequence disclosed herein, e.g., the amino acid sequence of SEQ ID NO: 2, or an amino acid sequence having at least 80, 85, 90, 95, or 99% identity thereto. In some embodiments, the transmembrane domain comprises a CD8 transmembrane domain. In some embodiments, the transmembrane domain comprises a transmembrane domain sequence disclosed herein, e.g., the amino acid sequence of SEQ ID NO: 6, or an amino acid sequence having at least 80, 85, 90, 95, or 99% identity thereto. In some embodiments, the intracellular signaling domain comprises a 4-1BB intracellular domain. In some embodiments, the intracellular signaling domain comprises a costimulatory signaling domain sequence disclosed herein, e.g., the amino acid sequence of SEQ ID NO: 7, or an amino acid sequence having at least 80, 85, 90, 95, or 99% identity thereto. In some embodiments, the intracellular signaling domain comprises a CD3 intracellular domain. In some embodiments, the intracellular signaling domain comprises a primary signaling domain sequence disclosed herein, e.g., the amino acid sequence of SEQ ID NO: 9 or 10, or an amino acid sequence having at least 80, 85, 90, 95, or 99% identity thereto.

Exemplary diabody sequences are disclosed in Table 31. In some embodiments, the CAR comprises an antigen binding domain comprising an amino acid sequence selected from the group consisting of SEQ ID NOs: 321-330, or an amino acid sequence having at least 80, 85, 90, 95, or 99% identity thereto. In some embodiments, the CAR comprises an amino acid sequence selected from the group consisting of SEQ ID NOs: 339-348, or an amino acid sequence having at least 80, 85, 90, 95, or 99% identity thereto.

TABLE 31
Exemplary components of diabody CARs.

| SEQ | | |
|---|---|---|
| ID NO | Description | Amino acid sequence* |
| SEQ ID | JL1 antigen | QVQLQESGPGLVKPSETLSLTCTVSGVSLPDYGVSWIRQPPGKCLEWIGVIWGS |
| NO: 321 | binding | ETTYYQSSLKSRVTISKDNSKNQVSLKLSSVTAADTAVYYCAKHYYYGGSYAMDY |
| | domain | WGQGTLVTVSSGGGGSQSALTQPASVSGSPGQSITISCTGTSSDVGGYNYVS |
| | | WYQQHPGKAPKLMIYDVSNRPSGVSNRESGSKSGNTASLTISGLQAEDEAD |
| | | YYCSSYTSSSTLYVFGSGTKVTVLGGGGSGGGGSGGGGSGGGGSQVQLQE |
| | | SGGGVVQPGRSLRLSCAASGFTFSSYGMHWVRQAPGKGLEWVAVISYDGS |
| | | NKYYADSVKGRFTISRDNSKNTLYLQMNSLRAEDTAVYYCGGSGYALHD |
| | | DYYGLDVWGQGTLVTVSSGGGGSEIVMTQSPATLSLSPGERATLSCRASQDIS |
| | | KYLNWYQQKPGQAPRLLIYHTSRLHSGIPARFSGSGSGTDYTLTISSLQPEDFAV |
| | | YFCQQGNTLPYTFGCGTKLEIK |
| SEQ ID | JL1 full | QVQLQESGPGLVKPSETLSLTCTVSGVSLPDYGVSWIRQPPGKCLEWIGVI |
| NO: 339 | length | WGSETTYYQSSLKSRVTISKDNSKNQVSLKLSSVTAADTAVYYCAKHYYY |
| | diabody | GGSYAMDYWGQGTLVTVSSGGGGSQSALTQPASVSGSPGQSITISCTGTSS |
| | CAR | DVGGYNYVSWYQQHPGKAPKLMIYDVSNRPSGVSNRFSGSKSGNTASLTI |
| | | SGLQAEDEADYYCSSYTSSSTLYVFGSGTKVTVLGGGGSGGGGSGGGGSG |
| | | GGGSQVQLQESGGGVVQPGRSLRLSCAASGFTFSSYGMHWVRQAPGKGL |
| | | EWVAVISYDGSNKYYADSVKGRFTISRDNSKNTLYLQMNSLRAEDTAVYY |
| | | CGGSGYALHDDYYGLDVWGQGTLVTVSSGGGGSEIVMTQSPATLSLSPGE |
| | | RATLSCRASQDISKYLNWYQQKPGQAPRLLIYHTSRLHSGIPARFSGSGSGT |
| | | DYTLTISSLQPEDFAVYFCQQGNTLPYTFGCGTKLEIKTTTPAPRPPTPAPTI |
| | | ASQPLSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKESRS |
| | | ADAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDALH |
| | | MQALPPR |
| SEQ ID | JL2 antigen | EIVMTQSPATLSLSPGERATLSCRASQDISKYLNWYQQKPGQAPRLLIYHTSRLHS |
| NO: 322 | binding | GIPARFSGSGSGTDYTLTISSLQPEDFAVYFEFCQQGNTLPYTFGCGTKLEIKGGGG |
| | domain | SQVQLQESGGGVVQPGRSLRLSCAASGFTFSSYGMHWVRQAPGKGLEWV |
| | | AVISYDGSNKYYADSVKGRFTISRDNSKNTLYLQMNSLRAEDTAVYYCGG |
| | | SGYALHDDYYGLDVWGQGTLVTVSSGGGGSGGGGSGGGGSGGGGSQSA |
| | | LTQPASVSGSPGQSITISCTGTSSDVGUGYNYVSWYQQHPGKAPKLMIYDVS |
| | | NRPSGVSNRFSGSKSGNTASLTISGLQAEDEADYYCSSYTSSSTLYVFEGSGT |
| | | KVTVLGGGGSQVQLQESGPGLVKPSETLSLTCTVSGVSLPDYGVSWIRQPPGK |
| | | CLEWIGVIWGSETTYYQSSLKSRVTISKDNSKNQVSLKLSSVTAADTAVYYCAKHY |
| | | YYGGSYAMDYWGQGTLVTVSS |
| SEQ ID | JL2 full | EIVMTQSPATLSLSPGERATLSCRASQDISKYLNWYQQKPGQAPRLLIYHTS |
| NO: 340 | length | RLHSGIPARFSGSGSGTDYTLTISSLQPEDFAVYFCQQGNTLPYTFGCGTKL |
| | diabody CAR | EIKGGGGSQVQLQESGGGVVQPGRSLRLSCAASGFTFSSYGMHWVRQAPG |
| | | KGLEWVAVISYDGSNKYYADSVKGRFTISRDNSKNTLYLQMNSLRAEDTA |
| | | VYYCGGSGYALHDDYYGLDVWGQGTLVTVSSGGGGSGGGGSGGGGSGG |
| | | GGSQSALTQPASVSGSPGQSITISCTGTSSDVGGYNYVSWYQQHPGKAPKL |
| | | MIYDVSNRPSGVSNRFSGSKSGNTASLTISGLQAEDEADYYCSSYTSSSTLY |
| | | VFGSGTKVTVLGGGGSQVQLQESGPGLVKPSETLSLTCTVSGVSLPDYGVS |
| | | WIRQPPGKCLEWIGVIWGSETTYYQSSLKSRVTISKDNSKNQVSLKLSSVTA |
| | | ADTAVYYCAKHYYYGGSYAMDYWGQGTLVTVSSTTTPAPRPPTPAPTIAS |
| | | QPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDALH |
| | | MQALPPR |
| SEQ ID | JL3 antigen | QVQLQESGPGLVKPSETLSLTCTVSGVSLPDYGVSWIRQPPGKCLEWIGVIWGS |
| NO: 323 | binding | ETTYYQSSLKSRVTISKDNSKNQVSLKLSSVTAADTAVYYCAKHYYYGGSYAMDY |
| | domain | WGQGTLVTVSSGGGGSQVQLQESGGGVVQPGRSLRLSCAASGFTFSSYGMH |
| | | WVRQAPGKGLEWVAVISYDGSNKYYADSVKGRFTISRDNSKNTLYLQMN |
| | | SLRAEDTAVYYCGGSGYALHDDYYGLDVWGQGTLVTVSSGGGGSGGGG |
| | | SGGGGSGGGGSQSALTQPASVSGSPGQSITISCTGTSSDVGGYNYVSWYQQ |
| | | HPGKAPKLMIYDVSNRPSGVSNRFSGSKSGNTASLTISGLQAEDEADYYCS |
| | | SYTSSSTLYVEFGSGTKVTVLGGGGSEIVMTQSPATLSILSPGERATLSCRASQDI |
| | | SKYLNWYQQKPGQAPRLLIYHTSRLHSGIPARFSGSGSGTDYTLTISSLQPEDFAV |
| | | YFCQQGNTLPYTFGCGTKLEIK |
| SEQ ID | JL3 full | QVQLQESGPGLVKPSETLSLTCTVSGVSLPDYGVSWIRQPPGKCLEWIGVI |
| NO: 341 | length | WGSETTYYQSSLKSRVTISKDNSKNQVSLKLSSVTAADTAVYYCAKHYYY |
| | diabody CAR | GGSYAMDYWGQGTLVTVSSGGGGSQVQLQESGGGVVQPGRSLRLSCAAS |
| | | GFTFSSYGMHWVRQAPGKGLEWVAVISYDGSNKYYADSVKGRFTISRDNS |
| | | KNTLYLQMNSLRAEDTAVYYCGGSGYALHDDYYGLDVWGQGTLVTVSS |
| | | GGGGSGGGGSGGGGSGGGGSQSALTQPASVSGSPGQSITISCTGTSSDVGG |
| | | YNYVSWYQQHPGKAPKLMIYDVSNRPSGVSNRFSGSKSGNTASLTISGLQ |
| | | AEDEADYYCSSYTSSSTLYVFGSGTKVTVLGGGGSEIVMTQSPATLSLSPGE |
| | | RATLSCRASQDISKYLNWYQQKPGQAPRLLIYHTSRLHSGIPARFSGSGSGT |
| | | DYTLTISSLQPEDFAVYFCQQGNTLPYTFGCGTKLEIKTTTPAPRPPTPAPTI |
| | | ASQPLSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDALH |
| | | MQALPPR |
| SEQ ID | JL4 antigen | EIVMTQSPATLSLSPGERATLSCRASQDISKYLNWYQQKPGQAPRLLIYHTSRLHS |
| NO: 324 | binding | GIPARFSGSGSGTDYTLTISSLQPEDFAVYFEFCQQGNTLPYTFGCGTKLEIKGGGG |
| | domain | SQSALTQPASVSGSPGQSITISCTGTSSDVGGYNYVSWYQQHPGKAPKLMI |
| | | YDVSNRPSGVSNRESGSKSGNTASLTISGLQAEDEADYYCSSYTSSSTLYVF |
| | | GSGTKVTVLGGGGSGGGGSGGGGSGGGGSQVQLQESGGGVVQPGRSLRL |
| | | SCAASGFTFSSYGMHWVRQAPGKGLEWVAVISYDGSNKYYADSVKGRFTI |
| | | SRDNSKNTLYLQMNSLRAEDTAVYYCGGSGYALHDDYYGLDVWGQGTL |
| | | VTVSSGGGGSQVQLQESGPGLVKPSETLSLTCTVSGVSLPDYGVSWIRQPPGK |
| | | CLEWIGVIWGSETTYYQSSLKSRVTISKDNSKNQVSLKLSSVTAADTAVYYCAKHY |
| | | YYGGSYAMDYWGQGTLVTVSS |
| SEQ ID | JL4 full | EIVMTQSPATLSLSPGERATLSCRASQDISKYLNWYQQKPGQAPRLLIYHTS |
| NO: 342 | length | RLHSGIPARFSGSGSGTDYTLTISSLQPEDFAVYFCQQGNTLPYTFGCGTKL |
| | diabody CAR | EIKGGGGSQSALTQPASVSGSPGQSITISCTGTSSDVGGYNYVSWYQQHPG |
| | | KAPKLMIYDVSNRPSGVSNRFSGSKSGNTASLTISGLQAEDEADYYCSSYTS |
| | | SSTLYVFGSGTKVTVLGGGGSGGGGSGGGGSGGGGSQVQLQESGGGVVQ |
| | | PGRSLRLSCAASGFTFSSYGMHWVRQAPGKGLEWVAVISYDGSNKYYADS |
| | | VKGRFTISRDNSKNTLYLQMNSLRAEDTAVYYCGGSGYALHDDYYGLDV |
| | | WGQGTLVTVSSGGGGSQVQLQESGPGLVKPSETLSLTCTVSGVSLPDYGVS |
| | | WIRQPPGKCLEWIGVIWGSETTYYQSSLKSRVTISKDNSKNQVSLKLSSVTA |
| | | ADTAVYYCAKHYYYGGSYAMDYWGQGTLVTVSSTTTPAPRPPTPAPTIAS |
| | | QPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDALH |
| | | MQALPPR |
| SEQ ID | JL5 antigen | QVQLQESGGGVVQPGRSLRLSCAASGFTFSSYGMHWVRQAPGKCLEWVA |
| NO: 325 | binding | VISYDGSNKYYADSVKGRFTISRDNSKNTLYLQMNSLRAEDTAVYYCGGS |
| | domain | GYALHDDYYGLDVWGQGTLVTVSSGGGGSEIVMTQSPATLSLSPGERATLS |
| | | CRASQDISKYLNWYQQKPGQAPRLLIYHTSRLHSGIPARFSGSGSGTDYTLTISSL |
| | | QPEDFAVYEFCQQGNTLPYTFGQGTKLEIKGGGGSGGGGSGGGGSGGGGSQV |
| | | QLQESGPGLVKPSETLSLTCTVSGVSLPDYGVSWIRQPPGKGLEWIGVIWGSETT |
| | | YYQSSLKSRVTISKDNSKNQVSLKLSSVTAADTAVYYCAKHYYYGGSYAMDYWGQ |
| | | GTLVTVSSGGGGSQSALTQPASVSGSPGQSITISCTGTSSDVGGYNYVSWYQ |
| | | QHPGKAPKLMIYDVSNRPSGVSNRESGSKSGNTASLTISGLQAEDEADYYC |
| | | SSYTSSSTLYVFGCGTKVTVL |
| SEQ ID | JL5 full | QVQLQESGGGVVQPGRSLRLSCAASGFTFSSYGMHWVRQAPGKCLEWVA |
| NO: 343 | length | VISYDGSNKYYADSVKGRFTISRDNSKNTLYLQMNSLRAEDTAVYYCGGS |
| | diabody CAR | GYALHDDYYGLDVWGQGTLVTVSSGGGGSEIVMTQSPATLSLSPGERATL |
| | | SCRASQDISKYLNWYQQKPGQAPRLLIYHTSRLHSGIPARFSGSGSGTDYTL |
| | | TISSLQPEDFAVYFCQQGNTLPYTFGQGTKLEIKGGGGSGGGGSGGGGSGG |
| | | GGSQVQLQESGPGLVKPSETLSLTCTVSGVSLPDYGVSWIRQPPGKGLEWI |
| | | GVIWGSETTYYQSSLKSRVTISKDNSKNQVSLKLSSVTAADTAVYYCAKH |
| | | YYYGGSYAMDYWGQGTLVTVSSGGGGSQSALTQPASVSGSPGQSITISCT |
| | | GTSSDVGGYNYVSWYQQHPGKAPKLMIYDVSNRPSGVSNRFSGSKSGNTA |
| | | SLTISGLQAEDEADYYCSSYTSSSTLYVFGCGTKVTVLTTTPAPRPPTPAPTI |
| | | ASQPLSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDALH |
| | | MQALPPR |
| SEQ ID | JL6 antigen | QSALTQPASVSGSPGQSITISCTGTSSDVGGYNYVSWYQQHPGKAPKLMIY |
| NO: 326 | binding | DVSNRPSGVSNRESGSKSGNTASLTISGLQAEDEADYYCSSYTSSSTLYVFG |
| | domain | CGTKVTVLGGGGSQVQLQESGPGLVKPSETLSLTCTVSGVSLPDYGVSWIRQP |
| | | PGKGLEWIGVIWGSETTYYQSSLKSRVTISKDNSKNQVSLKLSSVTAADTAVYYCA |
| | | KHYYYGGSYAMDYWGQGTLVTVSSGGGGSGGGGSGGGGSGGGGSEIVMTQS |
| | | PATLSLSPGERATLSCRASQDISKYLNWYQQKPGQAPRLLIYHTSRLHSGIPARFS |
| | | GSGSGTDYTLTISSLQPEDFAVYFCQQGNTLPYTFGQGTKLEIKGGGGSQVQL |
| | | QESGGGVVQPGRSLRLSCAASGFTFSSYGMHWVRQAPGKCLEWVAVISYD |
| | | GSNKYYADSVKGRFTISRDNSKNTLYLQMNSLRAEDTAVYYCGGSGYAL |
| | | HDDYYGLDVWGQGTLVTVSS |
| SEQ ID | JL6 full | QSALTQPASVSGSPGQSITISCTGTSSDVGGYNYVSWYQQHPGKAPKLMIY |
| NO: 344 | length | DVSNRPSGVSNRFSGSKSGNTASLTISGLQAEDEADYYCSSYTSSSTLYVFG |
| | diabody CAR | CGTKVTVLGGGGSQVQLQESGPGLVKPSETLSLTCTVSGVSLPDYGVSWIR |
| | | QPPGKGLEWIGVIWGSETTYYQSSLKSRVTISKDNSKNQVSLKLSSVTAAD |
| | | TAVYYCAKHYYYGGSYAMDYWGQGTLVTVSSGGGGSGGGGSGGGGSGG |
| | | GGSEIVMTQSPATLSLSPGERATLSCRASQDISKYLNWYQQKPGQAPRLLIY |
| | | HTSRLHSGIPARFSGSGSGTDYTLTISSLQPEDFAVYFCQQGNTLPYTFGQG |
| | | TKLEIKGGGGSQVQLQESGGGVVQPGRSLRLSCAASGFTFSSYGMHWVRQ |
| | | APGKCLEWVAVISYDGSNKYYADSVKGRFTISRDNSKNTLYLQMNSLRAE |
| | | DTAVYYCGGSGYALHDDYYGLDVWGQGTLVTVSSTTTPAPRPPTPAPTIA |
| | | SQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDALH |
| | | MQALPPR |
| SEQ ID | JL7 antigen | QVQLQESGGGVVQPGRSLRLSCAASGFTFSSYGMHWVRQAPGKCLEWVA |
| NO: 327 | binding | VISYDGSNKYYADSVKGRFTISRDNSKNTLYLQMNSLRAEDTAVYYCGGS |
| | domain | GYALHDDYYGLDVWGQGTLVTVSSGGGGSQVQLQESGPGLVKPSETLSLTC |
| | | TVSGVSLPDYGVSWIRQPPGKGLEWIGVIWGSETTYYQSSLKSRVTISKDNSKNQ |
| | | VSLKLSSVTAADTAVYYCAKHYYYGGSYAMDYWGQGTLVTVSSGGGGSGGGGS |
| | | GGGGSGGGGSEIVMTQSPATLSLSPGERATLSCRASQDISKYLNWYQQKPGQA |
| | | PRLLIYVHTSRLHSGIPARFSGSGSGTDYTLTISSLQPEDFAVYFCQQGNTLPYTFG |
| | | QGTKLEIKGGGGSQSALTQPASVSGSPGQSITISCTGTSSDVGGYNYVSWYQ |
| | | QHPGKAPKLMIYDVSNRPSGVSNRESGSKSGNTASLTISGLQAEDEADYYC |
| | | SSYTSSSTLYVFGCGTKVTVL |
| SEQ ID | JL7 full | QVQLQESGGGVVQPGRSLRLSCAASGFTFSSYGMHWVRQAPGKCLEWVA |
| NO: 345 | length | VISYDGSNKYYADSVKGRFTISRDNSKNTLYLQMNSLRAEDTAVYYCGGS |
| | diabody CAR | GYALHDDYYGLDVWGQGTLVTVSSGGGGSQVQLQESGPGLVKPSETLSLT |
| | | CTVSGVSLPDYGVSWIRQPPGKGLEWIGVIWGSETTYYQSSLKSRVTISKD |
| | | NSKNQVSLKLSSVTAADTAVYYCAKHYYYGGSYAMDYWGQGTLVTVSS |
| | | GGGGSGGGGSGGGGSGGGGSEIVMTQSPATLSLSPGERATLSCRASQDISK |
| | | YLNWYQQKPGQAPRLLIYHTSRLHSGIPARFSGSGSGTDYTLTISSLQPEDF |
| | | AVYFCQQGNTLPYTFGQGTKLEIKGGGGSQSALTQPASVSGSPGQSITISCT |
| | | GTSSDVGGYNYVSWYQQHPGKAPKLMIYDVSNRPSGVSNRFSGSKSGNTA |
| | | SLTISGLQAEDEADYYCSSYTSSSTLYVFGCGTKVTVLTTTPAPRPPTPAPTI |
| | | ASQPLSLRPEACRPAAGGAVHTRGLDFACDIYIWAPLAGTCGVLLLSLVITL |
| | | YCKRGRKKLLYIFKQPFMRPVQTTQEEDGCSCRFPEEEEGGCELRVKFSRS |
| | | ADAPAYKQGQNQLYNELNLGRREEYDVLDKRRGRDPEMGGKPRRKNPQE |
| | | GLYNELQKDKMAEAYSEIGMKGERRRGKGHDGLYQGLSTATKDTYDALH |
| | | MQALPPR |
| SEQ ID | JL8 antigen | QSALTQPASVSGSPGQSITISCTGTSSDVGGYNYVSWYQQHPGKAPKLMIY |
| NO: 328 | binding | DVSNRPSGVSNRESGSKSGNTASLTISGLQAEDEADYYCSSYTSSSTLYVFG |
| | domain | CGTKVTVLGGGGSEIVMTQSPATLSLSPGERATLSCRASQDISKYLNWYQQKP |
| | | GQAPRLLIYHTSRLHSGIPARFSGSGSGTDYTLTISSLQPEDFAVYFCQQGNTLPY |
| | | TFGQGTKLEIKGGGGSGGGGSGGGGSGGGGSQVQLQESGPGLVKPSETLSLT |
| | | CTVSGVSLPDYGVSWIRQPPGKGLEWIGVIWGSETTYYQSSLKSRVTISKDNSKN |
| | | QVSLKLSSVTAADTAVYYCAKHYYYGGSYAMDYWGQGTLVTVSSGGGGSQVQL |
| | | QESGGGVVQPGRSLRLSCAASGFTFSSYGMHWVRQAPGKCLEWVAVISYD |
| | | GSNKYYADSVKGRFTISRDNSKNTLYLQMNSLRAEDTAVYYCGGSGYAL |
| | | HDDYYGLDVWGQGTLVTVSS |
| SEQ ID | JL8 full | QSALTQPASVSGSPGQSITISCTGTSSDVGGYNYVSWYQQHPGKAPKLMIY |
| NO: 346 | length | DVSNRPSGVSNRFSGSKSGNTASLTISGLQAEDEADYYCSSYTSSSTLYVFG |
| | diabody CAR | CGTKVTVLGGGGSEIVMTQSPATLSLSPGERATLSCRASQDISKYLNWYQQ |
| | | KPGQAPRLLIYHTSRLHSGIPARFSGSGSGTDYTLTISSLQPEDFAVYFCQQG |
| | | NTLPYTFGQGTKLEIKGGGGSGGGGSGGGGSGGGGSQVQLQESGPGLVKP |
| | | SETLSLTCTVSGVSLPDYGVSWIRQPPGKGLEWIGVIWGSETTYYQSSLKSR |
| | | VTISKDNSKNQVSLKLSSVTAADTAVYYCAKHYYYGGSYAMDYWGQGTL |
| | | VTVSSGGGGSQVQLQESGGGVVQPGRSLRLSCAASGFTFSSYGMHWVRQ |
| | | APGKCLEWVAVISYDGSNKYYADSVKGRFTISRDNSKNTLYLQMNSLRAE |
| | | DTAVYYCGGSGYALHDDYYGLDVWGQGTLVTVSSTTTPAPRPPTPAPTIA |
| | | SQPLSLRPEACRPAAGGAVHTRGLDFACDIYIWAPLAGTCGVLLLSLVITLY |
| | | CKRGRKKLLYIFKQPFMRPVQTTQEEDGCSCRFPEEEEGGCELRVKFSRSA |
| | | DAPAYKQGQNQLYNELNLGRREEYDVLDKRRGRDPEMGGKPRRKNPQEG |
| | | LYNELQKDKMAEAYSEIGMKGERRRGKGHDGLYQGLSTATKDTYDALH |
| | | MQALPPR |
| SEQ ID | JL9 antigen | QVQLQESGPGLVKPSETLSLTCTVSGVSLPDYGVSWIRQPPGKCLEWIGVIWGS |
| NO: 329 | binding | ETTYYQSSLKSRVTISKDNSKNQVSLKLSSVTAADTAVYYCAKHYYYGGSYAMDY |
| | domain | WGQGTLVTVSSGGGGSDIQMTQSPSSLSASVGDRVTITCRASQSISSYLNWY |
| | | QQKPGKAPKLLIYAASSLQSGVPSRESGSGSGTDFTLTISSLQPEDFATYYC |
| | | QQSYSTPLTFGQGTKVEIKGGGGSGGGGSGGGGSGGGGSEVQLLESGGGL |
| | | VQPGGSLRLSCAASGFTFSSYAMSWVRQAPGKGLEWVSAISGSGGSTYYA |
| | | DSVKGRFTISRDNSKNTLYLQMNSLRAEDTAVYYCARREWWGESWLFDY |
| | | WGQGTLVTVSSGGGGSEIVMTQSPATLSLSPGERATLSCRASQDISKYLNWYQ |
| | | QKPGQAPRLLIYHTSRLHSGIPARFSGSGSGTDYTLTISSLQPEDFAVYFCQQGN |
| | | TLPYTFGCGTKLEIK |
| SEQ ID | JL9 full | QVQLQESGPGLVKPSETLSLTCTVSGVSLPDYGVSWIRQPPGKCLEWIGVI |
| NO: 347 | length | WGSETTYYQSSLKSRVTISKDNSKNQVSLKLSSVTAADTAVYYCAKHYYY |
| | diabody CAR | GGSYAMDYWGQGTLVTVSSGGGGSDIQMTQSPSSLSASVGDRVTITCRAS |
| | | QSISSYLNWYQQKPGKAPKLLIYAASSLQSGVPSRFSGSGSGTDFTLTISSLQ |
| | | PEDFATYYCQQSYSTPLTFGQGTKVEIKGGGGSGGGGSGGGGSGGGGSEV |
| | | QLLESGGGLVQPGGSLRLSCAASGFTFSSYAMSWVRQAPGKGLEWVSAIS |
| | | GSGGSTYYADSVKGRFTISRDNSKNTLYLQMNSLRAEDTAVYYCARREW |
| | | WGESWLFDYWGQGTLVTVSSGGGGSEIVMTQSPATLSLSPGERATLSCRA |
| | | SQDISKYLNWYQQKPGQAPRLLIYHTSRLHSGIPARFSGSGSGTDYTLTISSL |
| | | QPEDFAVYFCQQGNTLPYTFGCGTKLEIKTTTPAPRPPTPAPTIASQPLSLRP |
| | | EACRPAAGGAVHTRGLDFACDIYIWAPLAGTCGVLLLSLVITLYCKRGRKK |
| | | LLYIFKQPFMRPVQTTQEEDGCSCRFPEEEEGGCELRVKFSRSADAPAYKQ |
| | | GQNQLYNELNLGRREEYDVLDKRRGRDPEMGGKPRRKNPQEGLYNELQK |
| | | DKMAEAYSEIGMKGERRRGKGHDGLYQGLSTATKDTYDALHMQALPPR |
| SEQ ID | JL10 antigen | EVQLLESGGGLVQPGGSLRLSCAASGFTFSSYAMSWVRQAPGKCLEWVSA |
| NO: 330 | binding | ISGSGGSTYYADSVKGRFTISRDNSKNTLYLQMNSLRAEDTAVYYCARRE |
| | domain | WWGESWLFDYWGQGTLVTVSSGGGGSEIVMTQSPATLSLSPGERATLSCRAS |
| | | QDISKYLNWYQQKPGQAPRLLIYHTSRLHSGIPARFSGSGSGTDYTLTISSLQPED |
| | | FAVYECQQGNTLPYTFGQGTKLEIKGGGGSGGGGSGGGGSGGGGSQVQLQE |
| | | SGPGLVKPSETLSLTCTVSGVSLPDYGVSWIRQPPGKGLEWIGVIWGSETTYYQS |
| | | SLKSRVTISKDNSKNQVSLKLSSVTAADTAVYYCAKHYYYGGSYAMDYWGQGTL |
| | | VTVSSGGGGSDIQMTQSPSSLSASVGDRVTITCRASQSISSYLNWYQQKPGK |
| | | APKLLIYAASSLQSGVPSRFSGSGSGTDFTLTISSLQPEDFATYYCQQSYSTP |
| | | LTFGCGTKVEIK |
| SEQ ID | JL10 full | EVQLLESGGGLVQPGGSLRLSCAASGFTFSSYAMSWVRQAPGKCLEWVSA |
| NO: 348 | length | ISGSGGSTYYADSVKGRFTISRDNSKNTLYLQMNSLRAEDTAVYYCARRE |
| | diabody CAR | WWGESWLFDYWGQGTLVTVSSGGGGSEIVMTQSPATLSLSPGERATLSCR |
| | | ASQDISKYLNWYQQKPGQAPRLLIYHTSRLHSGIPARFSGSGSGTDYTLTIS |
| | | SLQPEDFAVYFCQQGNTLPYTFGQGTKLEIKGGGGSGGGGSGGGGSGGGG |
| | | SQVQLQESGPGLVKPSETLSLTCTVSGVSLPDYGVSWIRQPPGKGLEWIGVI |
| | | WGSETTYYQSSLKSRVTISKDNSKNQVSLKLSSVTAADTAVYYCAKHYYY |
| | | GGSYAMDYWGQGTLVTVSSGGGGSDIQMTQSPSSLSASVGDRVTITCRAS |
| | | QSISSYLNWYQQKPGKAPKLLIYAASSLQSGVPSRFSGSGSGTDFTLTISSLQ |
| | | PEDFATYYCQQSYSTPLTFGCGTKVEIKTTTPAPRPPTPAPTIASQPLSLRPE |
| | | ACRPAAGGAVHTRGLDFACDIYIWAPLAGTCGVLLLSLVITLYCKRGRKKL |
| | | LYIFKQPFMRPVQTTQEEDGCSCRFPEEEEGGCELRVKFSRSADAPAYKQG |
| | | QNQLYNELNLGRREEYDVLDKRRGRDPEMGGKPRRKNPQEGLYNELQKD |
| | | KMAEAYSEIGMKGERRRGKGHDGLYQGLSTATKDTYDALHMQALPPR |
| SEQ ID | Anti-CD19 | QVQLQESGPGLVKPSETLSLTCTVSGVSLPDYGVSWIRQPPGKGLEWIGVI |
| NO: 250 | VH (CTL119) | WGSETTYYQSSLKSRVTISKDNSKNQVSLKLSSVTAADTAVYYCAKHYYY |
| | | GGSYAMDYWGQGTLVTVSS |
| SEQ ID | Anti-CD19 | EIVMTQSPATLSLSPGERATLSCRASQDISKYLNWYQQKPGQAPRLLIYHTS |
| NO: 251 | VL (CTL119) | RLHSGIPARFSGSGSGTDYTLTISSLQPEDFAVYFCQQGNTLPYTFGQGTKL |
| | | EIK |
| SEQ ID | Anti-CD19 | QVQLQESGPGLVKPSETLSLTCTVSGVSLPDYGVSWIRQPPGKCLEWIGVI |
| NO: 331 | VH (CTL119) | WGSETTYYQSSLKSRVTISKDNSKNQVSLKLSSVTAADTAVYYCAKHYYY |
| | variant | GGSYAMDYWGQGTLVTVSS |
| SEQ ID | Anti-CD19 | EIVMTQSPATLSLSPGERATLSCRASQDISKYLNWYQQKPGQAPRLLIYHTS |
| NO: 332 | VL (CTL119) | RLHSGIPARFSGSGSGTDYTLTISSLQPEDFAVYFCQQGNTLPYTFGCGTKL |
| | variant | EIK |
| SEQ ID | Anti-BCMA | QVQLQESGGGVVQPGRSLRLSCAASGFTFSSYGMHWVRQAPGKGLEWVA |
| NO: 93 | VH (PI61) | VISYDGSNKYYADSVKGRFTISRDNSKNTLYLQMNSLRAEDTAVYYCGGS |
| | | GYALHDDYYGLDVWGQGTLVTVSS |
| SEQ ID | Anti-BCMA | QSALTQPASVSGSPGQSITISCTGTSSDVGGYNYVSWYQQHPGKAPKLMIY |
| NO: 102 | VL (PI61) | DVSNRPSGVSNRFSGSKSGNTASLTISGLQAEDEADYYCSSYTSSSTLYVFG |
| | | SGTKVTVL |
| SEQ ID | Anti-BCMA | QVQLQESGGGVVQPGRSLRLSCAASGFTFSSYGMHWVRQAPGKCLEWVA |
| NO: 333 | VH (PI61) | VISYDGSNKYYADSVKGRFTISRDNSKNTLYLQMNSLRAEDTAVYYCGGS |
| | variant | GYALHDDYYGLDVWGQGTLVTVSS |
| SEQ ID | Anti-BCMA | QSALTQPASVSGSPGQSITISCTGTSSDVGGYNYVSWYQQHPGKAPKLMIY |
| NO: 334 | VL (PI61) | DVSNRPSGVSNRFSGSKSGNTASLTISGLQAEDEADYYCSSYTSSSTLYVFG |
| | variant | CGTKVTVL |
| SEQ ID | Anti-BCMA | EVQLLESGGGLVQPGGSLRLSCAASGFTFSSYAMSWVRQAPGKGLEWVSA |
| NO: 78 | VH (R1G5) | ISGSGGSTYYADSVKGRFTISRDNSKNTLYLQMNSLRAEDTAVYYCARRE |
| | | WWGESWLFDYWGQGTLVTVSS |
| SEQ ID | Anti-BCMA | DIQMTQSPSSLSASVGDRVTITCRASQSISSYLNWYQQKPGKAPKLLIYAAS |
| NO: 61 | VL (R1G5) | SLQSGVPSRFSGSGSGTDFTLTISSLQPEDFATYYCQQSYSTPLTFGQGTKVE |
| | | IK |
| SEQ ID | Anti-BCMA | EVQLLESGGGLVQPGGSLRLSCAASGFTFSSYAMSWVRQAPGKCLEWVSA |
| NO: 335 | VH (R1G5) | ISGSGGSTYYADSVKGRFTISRDNSKNTLYLQMNSLRAEDTAVYYCARRE |
| | variant | WWGESWLFDYWGQGTLVTVSS |
| SEQ ID | Anti-BCMA | DIQMTQSPSSLSASVGDRVTITCRASQSISSYLNWYQQKPGKAPKLLIYAAS |
| NO: 336 | VL (R1G5) | SLQSGVPSRFSGSGSGTDFTLTISSLQPEDFATYYCQQSYSTPLTFGCGTKVE |
| | variant | IK |
| SEQ ID | Linker | GGGGSGGGGS |
| NO: 5 | | |
| SEQ ID | Linker | GGGGSGGGGGGGGSGGGGS |
| NO: 63 | | |

*VH sequences are underlined and VL sequences are double-underlined. CD19-binding sequences (VH and VL) are shown in italic.

### Chimeric TCR

In one aspect, the antibodies and antibody fragments of the present invention can be grafted to one or more constant domain of a T cell receptor (“TCR”) chain, for example, a TCR alpha or TCR beta chain, to create a chimeric TCR. Without being bound by theory, it is believed that chimeric TCRs will signal through the TCR complex upon antigen binding. For example, a scFv as disclosed herein, can be grafted to the constant domain, e.g., at least a portion of the extracellular constant domain, the transmembrane domain and the cytoplasmic domain, of a TCR chain, for example, the TCR alpha chain and/or the TCR beta chain. As another example, an antibody fragment, for example a VL domain as described herein, can be grafted to the constant domain of a TCR alpha chain, and an antibody fragment, for example a VH domain as described herein, can be grafted to the constant domain of a TCR beta chain (or alternatively, a VL domain may be grafted to the constant domain of the TCR beta chain and a VH domain may be grafted to a TCR alpha chain). As another example, the CDRs of an antibody or antibody fragment, e.g., the CDRs of an antibody or antibody fragment as described herein may be grafted into a TCR alpha and/or beta chain to create a chimeric TCR. For example, the LCDRs disclosed herein may be grafted into the variable domain of a TCR alpha chain and the HCDRs disclosed herein may be grafted to the variable domain of a TCR beta chain, or vice versa. Such chimeric TCRs may be produced by methods known in the art (For example, Willemsen R A et al, Gene Therapy 2000; 7: 1369-1377; Zhang T et al, Cancer Gene Ther 2004; 11: 487-496; Aggen et al, Gene Ther. 2012 April; 19(4):365-74).

### Additional Embodiments

In one aspect, the CAR-expressing cell described herein can further comprise a second CAR, e.g., a second CAR that includes a different antigen binding domain, e.g., to the same target (BCMA) or a different target (e.g., CD19, CD20, or CS-1, or other multiple myeloma targets, e.g., kappa light chain, CD138, Lewis Y antigen, or CD38 (Garfall et al., Discovery Medicine, 2014, 17(91):37-46)). In one embodiment, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. While not wishing to be bound by theory, placement of a costimulatory signaling domain, e.g., 4-1BB, CD28, CD27 ICOS, or OX-40, onto the first CAR, and the primary signaling domain, e.g., CD3 zeta, on the second CAR can limit the CAR activity to cells where both targets are expressed. In one embodiment, the CAR expressing cell comprises a first BCMA CAR that includes a BCMA binding domain, a transmembrane domain and a costimulatory domain and a second CAR that targets an antigen other than BCMA (e.g., an antigen expressed on leukemia or lymphoma cells, e.g., CD19, CD20, CS-1, kappa light chain, CD139, Lewis Y antigen, or CD38) and includes an antigen binding domain, a transmembrane domain and a primary signaling domain. In another embodiment, the CAR expressing cell comprises a first BCMA CAR that includes a BCMA binding domain, a transmembrane domain and a primary signaling domain and a second CAR that targets an antigen other than BCMA (e.g., an antigen expressed on leukemia or lymphoma cells, e.g., CD19, CD20, CS-1, kappa light chain, CD139, Lewis Y antigen, or CD38) and includes an antigen binding domain to the antigen, a transmembrane domain and a costimulatory signaling domain. In one embodiment, the CAR-expressing cell comprises a BCMA CAR described herein and a CAR that targets CD19 (CD19 CAR).

In one embodiment, the CAR-expressing cell comprises a BCMA CAR described herein and an inhibitory CAR. In one embodiment, the inhibitory CAR comprises an antigen binding domain that binds an antigen found on normal cells but not cancer cells. In one embodiment, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta.

In one embodiment, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, e.g., as a fragment, e.g., an scFv, that does not form an association with the antigen binding domain of the second CAR, e.g., the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In one aspect, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

According to another aspect, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (e.g., selected by phage display).

It has also been discovered that cells having a plurality of chimeric membrane embedded receptors comprising an antigen binding domain that interactions between the antigen binding domain of the receptors can be undesirable, e.g., because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising antigen binding domains that minimize such interactions, as well as methods of making and using such cells and nucleic acids. In an embodiment the antigen binding domain of one of said first said second non-naturally occurring chimeric membrane embedded receptor, comprises an scFv, and the other comprises a single VH domain, e.g., a camelid, shark, or lamprey single VH domain, or a single VH domain derived from a human or mouse sequence.

In some embodiments, the claimed invention comprises a first and second CAR, wherein the antigen binding domain of one of said first CAR said second CAR does not comprise a variable light domain and a variable heavy domain. In some embodiments, the antigen binding domain of one of said first CAR said second CAR is an scFv, and the other is not an scFv. In some embodiments, the antigen binding domain of one of said first CAR said second CAR comprises a single VH domain, e.g., a camelid, shark, or lamprey single VH domain, or a single VH domain derived from a human or mouse sequence. In some embodiments, the antigen binding domain of one of said first CAR said second CAR comprises a nanobody. In some embodiments, the antigen binding domain of one of said first CAR said second CAR comprises a camelid VHH domain.

In some embodiments, the antigen binding domain of one of said first CAR said second CAR comprises an scFv, and the other comprises a single VH domain, e.g., a camelid, shark, or lamprey single VH domain, or a single VH domain derived from a human or mouse sequence. In some embodiments, the antigen binding domain of one of said first CAR said second CAR comprises an scFv, and the other comprises a nanobody. In some embodiments, the antigen binding domain of one of said first CAR said second CAR comprises an scFv, and the other comprises a camelid VHH domain.

In some embodiments, when present on the surface of a cell, binding of the antigen binding domain of said first CAR to its cognate antigen is not substantially reduced by the presence of said second CAR. In some embodiments, binding of the antigen binding domain of said first CAR to its cognate antigen in the presence of said second CAR is 85%, 90%, 95%, 96%, 97%, 98% or 99% of binding of the antigen binding domain of said first CAR to its cognate antigen in the absence of said second CAR.

In some embodiments, when present on the surface of a cell, the antigen binding domains of said first CAR said second CAR, associate with one another less than if both were scFv antigen binding domains. In some embodiments, the antigen binding domains of said first CAR said second CAR, associate with one another 85%, 90%, 95%, 96%, 97%, 98% or 99% less than if both were scFv antigen binding domains.

In another aspect, the CAR-expressing cell described herein can further express another agent, e.g., an agent which enhances the activity of a CAR-expressing cell. For example, in one embodiment, the agent can be an agent which inhibits an inhibitory molecule, e.g., an agent described herein. Inhibitory molecules, e.g., PD1, can, in some embodiments, decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta, or a fragment of any of these (e.g., at least a portion of an extracellular domain of any of these), and a second polypeptide which is an intracellular signaling domain described herein (e.g., comprising a costimulatory domain (e.g., 41BB, CD27 ICOS, or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of an extracellular domain of PD1), and a second polypeptide of an intracellular signaling domain described herein (e.g., a CD28 signaling domain described herein and/or a CD3 zeta signaling domain described herein). In embodiments, the CAR-expressing cell described herein comprises a switch costimulatory receptor, e.g., as described in WO 2013/019615, which is incorporated herein by reference in its entirety. PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In one embodiment, the agent comprises the extracellular domain (ECD) of an inhibitory molecule, e.g., Programmed Death 1 (PD1), can be fused to a transmembrane domain and intracellular signaling domains such as 41BB and CD3 zeta (also referred to herein as a PD1 CAR). In one embodiment, the PD1 CAR, when used in combinations with a BCMA CAR described herein, improves the persistence of the CAR-expressing cell, e.g., T cell or NK cell. In one embodiment, the CAR is a PD1 CAR comprising the extracellular domain of PD1 indicated as underlined in SEQ ID NO: 24. In one embodiment, the PD1 CAR comprises the amino acid sequence of SEQ ID NO: 24.

In one embodiment, the PD1 CAR comprises the amino acid sequence provided below (SEQ ID NO: 22).

In one embodiment, the agent comprises a nucleic acid sequence encoding the PD1 CAR, e.g., the PD1 CAR described herein. In one embodiment, the nucleic acid sequence for the PD1 CAR is provided as SEQ ID NO: 23, with the PD1 ECD underlined.

In another aspect, the present invention provides a population of CAR-expressing cells, e.g., CART cells or CAR-expressing NK cells. In some embodiments, the population of CAR-expressing cells comprises a mixture of cells expressing different CARs. For example, in one embodiment, the population of CAR-expressing cells (e.g., CART cells or CAR-expressing NK cells) can include a first cell expressing a CAR having an anti-BCMA binding domain described herein, and a second cell expressing a CAR having a different anti-BCMA binding domain, e.g., an anti-BCMA binding domain described herein that differs from the anti-BCMA binding domain in the CAR expressed by the first cell. As another example, the population of CAR-expressing cells can include a first cell expressing a CAR that includes an anti-BCMA binding domain, e.g., as described herein, and a second cell expressing a CAR that includes an antigen binding domain to a target other than BCMA (e.g., CD19, CD20, CS-1, kappa light chain, CD139, Lewis Y antigen, or CD38). In one embodiment, the population of CAR-expressing cells includes a first cell expressing a CAR comprising an anti-BCMA binding domain, e.g., as described herein, and a second cell expressing a CAR comprising an antigen binding domain that targets CD19 (CD19 CAR). In one embodiment, the population of CAR-expressing cells includes, e.g., a first cell expressing a CAR that includes a primary intracellular signaling domain, and a second cell expressing a CAR that includes a secondary signaling domain.

In another aspect, the present invention provides a population of cells wherein at least one cell in the population expresses a CAR having an anti-BCMA domain described herein, and a second cell expressing another agent, e.g., an agent which enhances the activity of a CAR-expressing cell. For example, in one embodiment, the agent can be an agent which inhibits an inhibitory molecule. Inhibitory molecules, e.g., can, in some embodiments, decrease the ability of a CAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta. In one embodiment, the agent which inhibits an inhibitory molecule comprises a first polypeptide, e.g., an inhibitory molecule, associated with a second polypeptide that provides a positive signal to the cell, e.g., an intracellular signaling domain described herein. In one embodiment, the agent comprises a first polypeptide, e.g., of an inhibitory molecule such as PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta, or a fragment of any of these (e.g., at least a portion of an extracellular domain of any of these), and a second polypeptide which is an intracellular signaling domain described herein (e.g., comprising a costimulatory domain (e.g., 41BB, CD27, ICOS, or CD28, e.g., as described herein) and/or a primary signaling domain (e.g., a CD3 zeta signaling domain described herein). In one embodiment, the agent comprises a first polypeptide of PD1 or a fragment thereof (e.g., at least a portion of the extracellular domain of PD1), and a second polypeptide of an intracellular signaling domain described herein (e.g., a CD28 signaling domain described herein and/or a CD3 zeta signaling domain described herein).

In one aspect, the present invention provides methods comprising administering a population of CAR-expressing cells (e.g., CART cells or CAR-expressing NK cells), e.g., a mixture of cells expressing different CARs, in combination with another agent, e.g., a kinase inhibitor, such as a kinase inhibitor described herein. In another aspect, the present invention provides methods comprising administering a population of cells wherein at least one cell in the population expresses a CAR having an anti-cancer associated antigen binding domain as described herein, and a second cell expressing another agent, e.g., an agent which enhances the activity of a CAR-expressing cell, in combination with another agent, e.g., a kinase inhibitor, such as a kinase inhibitor described herein.

### Natural Killer Cell Receptor (NKR) CARs

In an embodiment, the CAR molecule described herein comprises one or more components of a natural killer cell receptor (NKR), thereby forming an NKR-CAR. The NKR component can be a transmembrane domain, a hinge domain, or a cytoplasmic domain from any of the following natural killer cell receptors: killer cell immunoglobulin-like receptor (KIR), e.g., KIR2DL1, KIR2DL2/L3, KIR2DL4, KIR2DL5A, KIR2DL5B, KIR2DS1, KIR2DS2, KIR2DS3, KIR2DS4, DIR2DS5, KIR3DL1/S1, KIR3DL2, KIR3DL3, KIR2DP1, and KIR3DP1; natural cytotoxicity receptor (NCR), e.g., NKp30, NKp44, NKp46; signaling lymphocyte activation molecule (SLAM) family of immune cell receptors, e.g., CD48, CD229, 2B4, CD84, NTB-A, CRACC, BLAME, and CD2F-10; Fc receptor (FcR), e.g., CD16, and CD64; and Ly49 receptors, e.g., LY49A, LY49C. The NKR-CAR molecules described herein may interact with an adaptor molecule or intracellular signaling domain, e.g., DAP12.

Exemplary configurations and sequences of CAR molecules comprising NKR components are described in International Publication No. WO2014/145252, the contents of which are hereby incorporated by reference.

### Non-Antibody Scaffolds

In embodiments, the antigen binding domain comprises a non-antibody scaffold, for example, a fibronectin, ankyrin, domain antibody, lipocalin, small modular immuno-pharmaceutical, maxybody, Protein A, or affilin. The non-antibody scaffold has the ability to bind to target antigen on a cell. In embodiments, the antigen binding domain is a polypeptide or fragment thereof of a naturally occurring protein expressed on a cell. In some embodiments, the antigen binding domain comprises a non-antibody scaffold. A wide variety of non-antibody scaffolds can be employed so long as the resulting polypeptide includes at least one binding region which specifically binds to the target antigen on a target cell.

Non-antibody scaffolds include: fibronectin (Novartis, MA), ankyrin (Molecular Partners AG, Zurich, Switzerland), domain antibodies (Domantis, Ltd., Cambridge, MA, and Ablynx nv, Zwijnaarde, Belgium), lipocalin (Pieris Proteolab AG, Freising, Germany), small modular immuno-pharmaceuticals (Trubion Pharmaceuticals Inc., Seattle, WA), maxybodies (Avidia, Inc., Mountain View, CA), Protein A (Affibody AG, Sweden), and affilin (gamma-crystallin or ubiquitin) (Scil Proteins GmbH, Halle, Germany).

### Strategies for Regulating Chimeric Antigen Receptors

There are many ways CAR activities can be regulated. In some embodiments, a regulatable CAR (RCAR) where the CAR activity can be controlled is desirable to optimize the safety and efficacy of a CAR therapy. For example, inducing apoptosis using, e.g., a caspase fused to a dimerization domain (see, e.g., Di et al., N Engl. J. Med. 2011 Nov. 3; 365(18):1673-1683), can be used as a safety switch in the CAR therapy of the instant invention. In another example, CAR-expressing cells can also express an inducible Caspase-9 (iCaspase-9) molecule that, upon administration of a dimerizer drug (e.g., rimiducid (also called AP1903 (Bellicum Pharmaceuticals) or AP20187 (Ariad)) leads to activation of the Caspase-9 and apoptosis of the cells. The iCaspase-9 molecule contains a chemical inducer of dimerization (CID) binding domain that mediates dimerization in the presence of a CID. This results in inducible and selective depletion of CAR-expressing cells. In some cases, the iCaspase-9 molecule is encoded by a nucleic acid molecule separate from the CAR-encoding vector(s). In some cases, the iCaspase-9 molecule is encoded by the same nucleic acid molecule as the CAR-encoding vector. The iCaspase-9 can provide a safety switch to avoid any toxicity of CAR-expressing cells. See, e.g., Song et al. Cancer Gene Ther. 2008; 15(10):667-75; Clinical Trial Id. No. NCT02107963; and Di Stasi et al. N. Engl. J. Med. 2011; 365:1673-83.

Alternative strategies for regulating the CAR therapy of the instant invention include utilizing small molecules or antibodies that deactivate or turn off CAR activity, e.g., by deleting CAR-expressing cells, e.g., by inducing antibody dependent cell-mediated cytotoxicity (ADCC). For example, CAR-expressing cells described herein may also express an antigen that is recognized by molecules capable of inducing cell death, e.g., ADCC or compliment-induced cell death. For example, CAR expressing cells described herein may also express a receptor capable of being targeted by an antibody or antibody fragment. Examples of such receptors include EpCAM, VEGFR, integrins (e.g., integrins αvβ3, α4, αI^{3/4}β3, α4β7, α5β1, αvβ3, αv), members of the TNF receptor superfamily (e.g., TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5 T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (e.g., versions preserving one or more extracellular epitopes but lacking one or more regions within the cytoplasmic domain). For example, CAR-expressing cells described herein may also express a truncated epidermal growth factor receptor (EGFR) which lacks signaling capacity but retains the epitope that is recognized by molecules capable of inducing ADCC, e.g., cetuximab (ERBITUX®), such that administration of cetuximab induces ADCC and subsequent depletion of the CAR-expressing cells (see, e.g., WO2011/056894, and Jonnalagadda et al., Gene Ther. 2013; 20(8)853-860). Another strategy includes expressing a highly compact marker/suicide gene that combines target epitopes from both CD32 and CD20 antigens in the CAR-expressing cells described herein, which binds rituximab, resulting in selective depletion of the CAR-expressing cells, e.g., by ADCC (see, e.g., Philip et al., Blood. 2014; 124(8)1277-1287). Other methods for depleting CAR-expressing cells described herein include administration of CAMPATH®, a monoclonal anti-CD52 antibody that selectively binds and targets mature lymphocytes, e.g., CAR-expressing cells, for destruction, e.g., by inducing ADCC. In other embodiments, CAR-expressing cells can be selectively targeted using a CAR ligand, e.g., an anti-idiotypic antibody. In some embodiments, the anti-idiotypic antibody can cause effector cell activity, e.g., ADCC or ADC activities, thereby reducing the number of CAR-expressing cells. In other embodiments, the CAR ligand, e.g., the anti-idiotypic antibody, can be coupled to an agent that induces cell killing, e.g., a toxin, thereby reducing the number of CAR-expressing cells. Alternatively, the CAR molecules themselves can be configured such that the activity can be regulated, e.g., turned on and off, as described below.

In some embodiments, a RCAR comprises a set of polypeptides, typically two in the simplest embodiments, in which the components of a standard CAR described herein, e.g., an antigen binding domain and an intracellular signaling domain, are partitioned on separate polypeptides or members. In some embodiments, the set of polypeptides include a dimerization switch that, upon the presence of a dimerization molecule, can couple the polypeptides to one another, e.g., can couple an antigen binding domain to an intracellular signaling domain. Additional description and exemplary configurations of such regulatable CARs are provided herein and in International Publication No. WO 2015/090229, hereby incorporated by reference in its entirety.

In an embodiment, an RCAR comprises two polypeptides or members: 1) an intracellular signaling member comprising an intracellular signaling domain, e.g., a primary intracellular signaling domain described herein, and a first switch domain; 2) an antigen binding member comprising an antigen binding domain, e.g., that targets a tumor antigen described herein, as described herein and a second switch domain. Optionally, the RCAR comprises a transmembrane domain described herein. In an embodiment, a transmembrane domain can be disposed on the intracellular signaling member, on the antigen binding member, or on both. (Unless otherwise indicated, when members or elements of an RCAR are described herein, the order can be as provided, but other orders are included as well. In other words, in an embodiment, the order is as set out in the text, but in other embodiments, the order can be different. E.g., the order of elements on one side of a transmembrane region can be different from the example, e.g., the placement of a switch domain relative to a intracellular signaling domain can be different, e.g., reversed).

In an embodiment, the first and second switch domains can form an intracellular or an extracellular dimerization switch. In an embodiment, the dimerization switch can be a homodimerization switch, e.g., where the first and second switch domain are the same, or a heterodimerization switch, e.g., where the first and second switch domain are different from one another.

In embodiments, an RCAR can comprise a “multi switch.” A multi switch can comprise heterodimerization switch domains or homodimerization switch domains. A multi switch comprises a plurality of, e.g., 2, 3, 4, 5, 6, 7, 8, 9, or 10, switch domains, independently, on a first member, e.g., an antigen binding member, and a second member, e.g., an intracellular signaling member. In an embodiment, the first member can comprise a plurality of first switch domains, e.g., FKBP-based switch domains, and the second member can comprise a plurality of second switch domains, e.g., FRB-based switch domains. In an embodiment, the first member can comprise a first and a second switch domain, e.g., a FKBP-based switch domain and a FRB-based switch domain, and the second member can comprise a first and a second switch domain, e.g., a FKBP-based switch domain and a FRB-based switch domain.

In an embodiment, the intracellular signaling member comprises one or more intracellular signaling domains, e.g., a primary intracellular signaling domain and one or more costimulatory signaling domains.

In an embodiment, the antigen binding member may comprise one or more intracellular signaling domains, e.g., one or more costimulatory signaling domains. In an embodiment, the antigen binding member comprises a plurality, e.g., 2 or 3 costimulatory signaling domains described herein, e.g., selected from 4-1BB, CD28, CD27, ICOS, and OX40, and in embodiments, no primary intracellular signaling domain. In an embodiment, the antigen binding member comprises the following costimulatory signaling domains, from the extracellular to intracellular direction: 4-1BB-CD27; 4-1BB-CD27; CD27-4-1BB; 4-1BB-CD28; CD28-4-1BB; OX40-CD28; CD28-OX40; CD28-4-1BB; or 4-1BB-CD28. In such embodiments, the intracellular binding member comprises a CD3zeta domain. In one such embodiment the RCAR comprises (1) an antigen binding member comprising, an antigen binding domain, a transmembrane domain, and two costimulatory domains and a first switch domain; and (2) an intracellular signaling domain comprising a transmembrane domain or membrane tethering domain and at least one primary intracellular signaling domain, and a second switch domain.

An embodiment provides RCARs wherein the antigen binding member is not tethered to the surface of the CAR cell. This allows a cell having an intracellular signaling member to be conveniently paired with one or more antigen binding domains, without transforming the cell with a sequence that encodes the antigen binding member. In such embodiments, the RCAR comprises: 1) an intracellular signaling member comprising: a first switch domain, a transmembrane domain, an intracellular signaling domain, e.g., a primary intracellular signaling domain, and a first switch domain; and 2) an antigen binding member comprising: an antigen binding domain, and a second switch domain, wherein the antigen binding member does not comprise a transmembrane domain or membrane tethering domain, and, optionally, does not comprise an intracellular signaling domain. In some embodiments, the RCAR may further comprise 3) a second antigen binding member comprising: a second antigen binding domain, e.g., a second antigen binding domain that binds a different antigen than is bound by the antigen binding domain; and a second switch domain.

Also provided herein are RCARs wherein the antigen binding member comprises bispecific activation and targeting capacity. In this embodiment, the antigen binding member can comprise a plurality, e.g., 2, 3, 4, or 5 antigen binding domains, e.g., scFvs, wherein each antigen binding domain binds to a target antigen, e.g. different antigens or the same antigen, e.g., the same or different epitopes on the same antigen. In an embodiment, the plurality of antigen binding domains are in tandem, and optionally, a linker or hinge region is disposed between each of the antigen binding domains. Suitable linkers and hinge regions are described herein.

An embodiment provides RCARs having a configuration that allows switching of proliferation. In this embodiment, the RCAR comprises: 1) an intracellular signaling member comprising: optionally, a transmembrane domain or membrane tethering domain; one or more co-stimulatory signaling domain, e.g., selected from 4-1BB, CD28, CD27, ICOS, and OX40, and a switch domain; and 2) an antigen binding member comprising: an antigen binding domain, a transmembrane domain, and a primary intracellular signaling domain, e.g., a CD3zeta domain, wherein the antigen binding member does not comprise a switch domain, or does not comprise a switch domain that dimerizes with a switch domain on the intracellular signaling member. In an embodiment, the antigen binding member does not comprise a co-stimulatory signaling domain. In an embodiment, the intracellular signaling member comprises a switch domain from a homodimerization switch. In an embodiment, the intracellular signaling member comprises a first switch domain of a heterodimerization switch and the RCAR comprises a second intracellular signaling member which comprises a second switch domain of the heterodimerization switch. In such embodiments, the second intracellular signaling member comprises the same intracellular signaling domains as the intracellular signaling member. In an embodiment, the dimerization switch is intracellular. In an embodiment, the dimerization switch is extracellular.

In any of the RCAR configurations described here, the first and second switch domains comprise a FKBP-FRB based switch as described herein.

Also provided herein are cells comprising an RCAR described herein. Any cell that is engineered to express a RCAR can be used as a RCARX cell. In an embodiment the RCARX cell is a T cell, and is referred to as a RCART cell. In an embodiment the RCARX cell is an NK cell, and is referred to as a RCARN cell.

Also provided herein are nucleic acids and vectors comprising RCAR encoding sequences. Sequence encoding various elements of an RCAR can be disposed on the same nucleic acid molecule, e.g., the same plasmid or vector, e.g., viral vector, e.g., lentiviral vector. In an embodiment, (i) sequence encoding an antigen binding member and (ii) sequence encoding an intracellular signaling member, can be present on the same nucleic acid, e.g., vector. Production of the corresponding proteins can be achieved, e.g., by the use of separate promoters, or by the use of a bicistronic transcription product (which can result in the production of two proteins by cleavage of a single translation product or by the translation of two separate protein products). In an embodiment, a sequence encoding a cleavable peptide, e.g., a P2A or F2A sequence, is disposed between (i) and (ii). In an embodiment, a sequence encoding an IRES, e.g., an EMCV or EV71 IRES, is disposed between (i) and (ii). In these embodiments, (i) and (ii) are transcribed as a single RNA. In an embodiment, a first promoter is operably linked to (i) and a second promoter is operably linked to (ii), such that (i) and (ii) are transcribed as separate mRNAs.

Alternatively, the sequence encoding various elements of an RCAR can be disposed on the different nucleic acid molecules, e.g., different plasmids or vectors, e.g., viral vector, e.g., lentiviral vector. E.g., the (i) sequence encoding an antigen binding member can be present on a first nucleic acid, e.g., a first vector, and the (ii) sequence encoding an intracellular signaling member can be present on the second nucleic acid, e.g., the second vector.

### Dimerization Switches

Dimerization switches can be non-covalent or covalent. In a non-covalent dimerization switch, the dimerization molecule promotes a non-covalent interaction between the switch domains. In a covalent dimerization switch, the dimerization molecule promotes a covalent interaction between the switch domains.

In an embodiment, the RCAR comprises a FKBP/FRAP, or FKBP/FRB-based dimerization switch. FKBP12 (FKBP, or FK506 binding protein) is an abundant cytoplasmic protein that serves as the initial intracellular target for the natural product immunosuppressive drug, rapamycin. Rapamycin binds to FKBP and to the large PI3K homolog FRAP (RAFT, mTOR). FRB is a 93 amino acid portion of FRAP, that is sufficient for binding the FKBP-rapamycin complex (Chen, J., Zheng, X. F., Brown, E. J. & Schreiber, S. L. (1995) Identification of an 11-kDa FKBP12-rapamycin-binding domain within the 289-kDa FKBP12-rapamycin-associated protein and characterization of a critical serine residue. Proc Natl Acad Sci USA 92: 4947-51.)

In embodiments, an FKBP/FRAP, e.g., an FKBP/FRB, based switch can use a dimerization molecule, e.g., rapamycin or a rapamycin analog.

An exemplary amino acid sequence of FKBP is as follows:

(SEQ ID NO: 275)
D V P D Y A S L G G P S S P K K K R K V S R G V Q
V E T I S P G D G R T F P K R G Q T C V V H Y T G
M L E D G K K F D S S R D R N K P F K F M L G K Q
E V I R G W E E G V A Q M S V G Q R A K L T I S P
D Y A Y G A T G H P G I I P P H A T L V F D V E L
L K L E T S Y

In embodiments, an FKBP switch domain can comprise a fragment of FKBP having the ability to bind with FRB, or a fragment or analog thereof, in the presence of rapamycin or a rapalog. In one embodiment, the FKBP switch domain comprises the amino acid sequence of:

(SEQ ID NO: 276)
V Q V E T I S P G D G R T F P K R G Q T C V V H Y
T G M L E D G K K F D S S R D R N K P F K F M L G
K Q E V I R G W E E G V A Q M S V G Q R A K L T I
S P D Y A Y G A T G H P G I I P P H A T L V F D V
E L L K L E T S

### The Amino Acid Sequence of FRB is as Follows:

(SEQ ID NO: 277)
ILWHEMWHEG LEEASRLYFG ERNVKGMFEV LEPLHAMMER
GPQTLKETSF NQAYGRDLME AQEWCRKYMK SGNVKDLTQA
WDLYYHVFRR ISK

“FKBP/FRAP, e.g., an FKBP/FRB, based switch” as that term is used herein, refers to a dimerization switch comprising: a first switch domain, which comprises an FKBP fragment or analog thereof having the ability to bind with FRB, or a fragment or analog thereof, in the presence of rapamycin or a rapalog, e.g., RAD001, and has at least 70, 75, 80, 85, 90, 95, 96, 97, 98, or 99% identity with, or differs by no more than 30, 25, 20, 15, 10, 5, 4, 3, 2, or 1 amino acid residues from, the FKBP sequence of SEQ ID NO: 275 or 276; and a second switch domain, which comprises an FRB fragment or analog thereof having the ability to bind with FRB, or a fragment or analog thereof, in the presence of rapamycin or a rapalog, and has at least 70, 75, 80, 85, 90, 95, 96, 97, 98, or 99% identity with, or differs by no more than 30, 25, 20, 15, 10, 5, 4, 3, 2, or 1 amino acid residues from, the FRB sequence of SEQ ID NO: 277. In an embodiment, a RCAR described herein comprises one switch domain comprises amino acid residues disclosed in SEQ ID NO: 275 (or SEQ ID NO: 276), and one switch domain comprises amino acid residues disclosed in SEQ ID NO: 277.

In embodiments, the FKBP/FRB dimerization switch comprises a modified FRB switch domain that exhibits altered, e.g., enhanced, complex formation between an FRB-based switch domain, e.g., the modified FRB switch domain, a FKBP-based switch domain, and the dimerization molecule, e.g., rapamycin or a rapalogue, e.g., RAD001. In an embodiment, the modified FRB switch domain comprises one or more mutations, e.g., 2, 3, 4, 5, 6, 7, 8, 9, 10 or more, selected from mutations at amino acid position(s) L2031, E2032, S2035, R2036, F2039, G2040, T2098, W2101, D2102, Y2105, and F2108, where the wild-type amino acid is mutated to any other naturally-occurring amino acid. In an embodiment, a mutant FRB comprises a mutation at E2032, where E2032 is mutated to phenylalanine (E2032F), methionine (E2032M), arginine (E2032R), valine (E2032V), tyrosine (E2032Y), isoleucine (E2032I), e.g., SEQ ID NO: 278, or leucine (E2032L), e.g., SEQ ID NO: 279. In an embodiment, a mutant FRB comprises a mutation at T2098, where T2098 is mutated to phenylalanine (T2098F) or leucine (T2098L), e.g., SEQ ID NO: 280. In an embodiment, a mutant FRB comprises a mutation at E2032 and at T2098, where E2032 is mutated to any amino acid, and where T2098 is mutated to any amino acid, e.g., SEQ ID NO: 281. In an embodiment, a mutant FRB comprises an E2032I and a T2098L mutation, e.g., SEQ ID NO: 282. In an embodiment, a mutant FRB comprises an E2032L and a T2098L mutation, e.g., SEQ ID NO: 283.

TABLE 18
Exemplary mutant FRB having
increased affinity for a dimerization molecule.

| FRB | | SEQ |
|---|---|---|
| mutant | Amino Acid Sequence | ID NO: |
| E2032I | ILWHEMWHEGLIEASRLYFGERNVKGMFEVL | 278 |
| mutant | EPLHAMMERGPQTLKETSFNQAYGRDLMEAQ | |
| | EWCRKYMKSGNVKDLTQAWDLYYHVFRRISK | |
| | TS | |
| E2032L | ILWHEMWHEGLLEASRLYFGERNVKGMFEVL | 279 |
| mutant | EPLHAMMERGPQTLKETSFNQAYGRDLMEAQ | |
| | EWCRKYMKSGNVKDLTQAWDLYYHVERRISK | |
| | TS | |
| T2098L | ILWHEMWHEGLEEASRLYFGERNVKGMFEVL | 280 |
| mutant | EPLHAMMERGPQTLKETSFNQAYGRDLMEAQ | |
| | EWCRKYMKSGNVKDLLQAWDLYYHVERRISK | |
| | TS | |
| E2032, | ILWHEMWHEGLXEASRLYFGERNVKGMFEVL | 281 |
| T2098 | EPLHAMMERGPQTLKETSFNQAYGRDLMEAQ | |
| mutant | EWCRKYMKSGNVKDLXQAWDLYYHVFRRISK | |
| | TS, wherein X is any amino | |
| | acid residue | |
| E2032I, | ILWHEMWHEGLIEASRLYFGERNVKGMFEVL | 282 |
| T2098L | EPLHAMMERGPQTLKETSFNQAYGRDLMEAQ | |
| mutant | EWCRKYMKSGNVKDLLQAWDLYYHVERRISK | |
| | TS | |
| E2032L, | ILWHEMWHEGLLEASRLYFGERNVKGMFEVL | 283 |
| T2098L | EPLHAMMERGPQTLKETSFNQAYGRDLMEAQ | |
| mutant | EWCRKYMKSGNVKDLLQAWDLYYHVFRRISK | |
| | TS | |

Other suitable dimerization switches include a GyrB-GyrB based dimerization switch, a Gibberellin-based dimerization switch, a tag/binder dimerization switch, and a halo-tag/snap-tag dimerization switch. Following the guidance provided herein, such switches and relevant dimerization molecules will be apparent to one of ordinary skill.

### Dimerization Molecule

Association between the switch domains is promoted by the dimerization molecule. In the presence of dimerization molecule interaction or association between switch domains allows for signal transduction between a polypeptide associated with, e.g., fused to, a first switch domain, and a polypeptide associated with, e.g., fused to, a second switch domain. In the presence of non-limiting levels of dimerization molecule signal transduction is increased by 1.1, 1.2, 1.3, 1.4, 1.5, 1.6, 1.7, 1.8, 1.9, 2, 5, 10, 50, 100 fold, e.g., as measured in a system described herein.

Rapamycin and rapamycin analogs (sometimes referred to as rapalogues), e.g., RAD001, can be used as dimerization molecules in a FKBP/FRB-based dimerization switch described herein. In an embodiment the dimerization molecule can be selected from rapamycin (sirolimus), RAD001 (everolimus), zotarolimus, temsirolimus, AP-23573 (ridaforolimus), biolimus and AP21967. Additional rapamycin analogs suitable for use with FKBP/FRB-based dimerization switches are further described in the section entitled “Combination Therapies”, or in the subsection entitled “Combination with a Low, Immune Enhancing, Dose of an mTOR inhibitor”.

### Split CAR

In some embodiments, the CAR-expressing cell uses a split CAR. The split CAR approach is described in more detail in publications WO2014/055442 and WO2014/055657, incorporated herein by reference. Briefly, a split CAR system comprises a cell expressing a first CAR having a first antigen binding domain and a costimulatory domain (e.g., 41BB), and the cell also expresses a second CAR having a second antigen binding domain and an intracellular signaling domain (e.g., CD3 zeta). When the cell encounters the first antigen, the costimulatory domain is activated, and the cell proliferates. When the cell encounters the second antigen, the intracellular signaling domain is activated and cell-killing activity begins. Thus, the CAR-expressing cell is only fully activated in the presence of both antigens. In embodiments the first antigen binding domain recognizes BCMA, e.g., comprises an antigen binding domain described herein, and the second antigen binding domain recognizes an antigen expressed on acute myeloid leukemia cells, e.g., CD123, CLL-1, CD34, FLT3, or folate receptor beta. In embodiments the first antigen binding domain recognizes BCMA, e.g., comprises an antigen binding domain described herein, and the second antigen binding domain recognizes an antigen expressed on B-cells, e.g., CD10, CD19, CD20, CD22, CD34, CD123, FLT-3, ROR1, CD79b, CD179b, or CD79a.

Co-Expression of CAR with Other Molecules or Agents

### Co-Expression of a Second CAR

In some embodiments, the CAR-expressing cell described herein can further comprise a second CAR, for example, a second CAR that includes a different antigen binding domain, for example, to the same target (for example, CD19) or a different target (for example, a target other than CD19, for example, a target described herein). In some embodiments, the CAR-expressing cell comprises a first CAR that targets a first antigen and includes an intracellular signaling domain having a costimulatory signaling domain but not a primary signaling domain, and a second CAR that targets a second, different, antigen and includes an intracellular signaling domain having a primary signaling domain but not a costimulatory signaling domain. Placement of a costimulatory signaling domain, for example, 4-1BB, CD28, CD27, OX-40 or ICOS, onto the first CAR, and the primary signaling domain, for example, CD3 zeta, on the second CAR can limit the CAR activity to cells where both targets are expressed. In some embodiments, the CAR expressing cell comprises a first CAR that includes an antigen binding domain, a transmembrane domain and a costimulatory domain and a second CAR that targets another antigen and includes an antigen binding domain, a transmembrane domain and a primary signaling domain. In some embodiments, the CAR expressing cell comprises a first CAR that includes an antigen binding domain, a transmembrane domain and a primary signaling domain and a second CAR that targets another antigen and includes an antigen binding domain to the antigen, a transmembrane domain and a costimulatory signaling domain.

In some embodiments, the CAR-expressing cell comprises an XCAR described herein and an inhibitory CAR. In some embodiments, the inhibitory CAR comprises an antigen binding domain that binds an antigen found on normal cells but not cancer cells, for example, normal cells that also express X. In some embodiments, the inhibitory CAR comprises the antigen binding domain, a transmembrane domain and an intracellular domain of an inhibitory molecule. For example, the intracellular domain of the inhibitory CAR can be an intracellular domain of PD1, PD-L1, PD-L2, CTLA4, TIM3, CEACAM (CEACAM-1, CEACAM-3, and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta).

In some embodiments, when the CAR-expressing cell comprises two or more different CARs, the antigen binding domains of the different CARs can be such that the antigen binding domains do not interact with one another. For example, a cell expressing a first and second CAR can have an antigen binding domain of the first CAR, for example, as a fragment, for example, an scFv, that does not form an association with the antigen binding domain of the second CAR, for example, the antigen binding domain of the second CAR is a VHH.

In some embodiments, the antigen binding domain comprises a single domain antigen binding (SDAB) molecules include molecules whose complementary determining regions are part of a single domain polypeptide. Examples include, but are not limited to, heavy chain variable domains, binding molecules naturally devoid of light chains, single domains derived from conventional 4-chain antibodies, engineered domains and single domain scaffolds other than those derived from antibodies. SDAB molecules may be any of the art, or any future single domain molecules. SDAB molecules may be derived from any species including, but not limited to mouse, human, camel, llama, lamprey, fish, shark, goat, rabbit, and bovine. This term also includes naturally occurring single domain antibody molecules from species other than Camelidae and sharks.

In some embodiments, an SDAB molecule can be derived from a variable region of the immunoglobulin found in fish, such as, for example, that which is derived from the immunoglobulin isotype known as Novel Antigen Receptor (NAR) found in the serum of shark. Methods of producing single domain molecules derived from a variable region of NAR (“IgNARs”) are described in WO 03/014161 and Streltsov (2005) Protein Sci. 14:2901-2909.

In some embodiments, an SDAB molecule is a naturally occurring single domain antigen binding molecule known as heavy chain devoid of light chains. Such single domain molecules are disclosed in WO 9404678 and Hamers-Casterman, C. et al. (1993) Nature 363:446-448, for example. For clarity reasons, this variable domain derived from a heavy chain molecule naturally devoid of light chain is known herein as a VHH or nanobody to distinguish it from the conventional VH of four chain immunoglobulins. Such a VHH molecule can be derived from Camelidae species, for example in camel, llama, dromedary, alpaca and guanaco. Other species besides Camelidae may produce heavy chain molecules naturally devoid of light chain; such VHHs are within the scope of the invention.

The SDAB molecules can be recombinant, CDR-grafted, humanized, camelized, de-immunized and/or in vitro generated (for example, selected by phage display).

It has also been discovered, that cells having a plurality of chimeric membrane embedded receptors comprising an antigen binding domain that interactions between the antigen binding domain of the receptors can be undesirable, for example, because it inhibits the ability of one or more of the antigen binding domains to bind its cognate antigen. Accordingly, disclosed herein are cells having a first and a second non-naturally occurring chimeric membrane embedded receptor comprising antigen binding domains that minimize such interactions. Also disclosed herein are nucleic acids encoding a first and a second non-naturally occurring chimeric membrane embedded receptor comprising an antigen binding domains that minimize such interactions, as well as methods of making and using such cells and nucleic acids. In some embodiments the antigen binding domain of one of the first and the second non-naturally occurring chimeric membrane embedded receptor, comprises an scFv, and the other comprises a single VH domain, for example, a camelid, shark, or lamprey single VH domain, or a single VH domain derived from a human or mouse sequence.

In some embodiments, a composition herein comprises a first and second CAR, wherein the antigen binding domain of one of the first and the second CAR does not comprise a variable light domain and a variable heavy domain. In some embodiments, the antigen binding domain of one of the first and the second CAR is an scFv, and the other is not an scFv. In some embodiments, the antigen binding domain of one of the first and the second CAR comprises a single VH domain, for example, a camelid, shark, or lamprey single VH domain, or a single VH domain derived from a human or mouse sequence. In some embodiments, the antigen binding domain of one of the first and the second CAR comprises a nanobody. In some embodiments, the antigen binding domain of one of the first and the second CAR comprises a camelid VHH domain.

In some embodiments, the antigen binding domain of one of the first and the second CAR comprises an scFv, and the other comprises a single VH domain, for example, a camelid, shark, or lamprey single VH domain, or a single VH domain derived from a human or mouse sequence. In some embodiments, the antigen binding domain of one of the first and the second CAR comprises an scFv, and the other comprises a nanobody. In some embodiments, the antigen binding domain of one of the first and the second CAR comprises an scFv, and the other comprises a camelid VHH domain.

In some embodiments, when present on the surface of a cell, binding of the antigen binding domain of the first CAR to its cognate antigen is not substantially reduced by the presence of the second CAR. In some embodiments, binding of the antigen binding domain of the first CAR to its cognate antigen in the presence of the second CAR is at least 85%, 90%, 95%, 96%, 97%, 98% or 99%, for example, 85%, 90%, 95%, 96%, 97%, 98% or 99% of binding of the antigen binding domain of the first CAR to its cognate antigen in the absence of the second CAR.

In some embodiments, when present on the surface of a cell, the antigen binding domains of the first and the second CAR, associate with one another less than if both were scFv antigen binding domains. In some embodiments, the antigen binding domains of the first and the second CAR, associate with one another at least 85%, 90%, 95%, 96%, 97%, 98% or 99% less than, for example, 85%, 90%, 95%, 96%, 97%, 98% or 99% less than if both were scFv antigen binding domains.

Co-Expression of an Agent that Enhances CAR Activity

In some embodiments, the CAR-expressing cell described herein can further express another agent, for example, an agent that enhances the activity or fitness of a CAR-expressing cell.

For example, in some embodiments, the agent can be an agent which inhibits a molecule that modulates or regulates, for example, inhibits, T cell function. In some embodiments, the molecule that modulates or regulates T cell function is an inhibitory molecule. Inhibitory molecules, for example, PD1, can, in some embodiments, decrease the ability of a CAR-expressing cell to mount an immune effector response. Examples of inhibitory molecules include PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta.

In embodiments, an agent, for example, an inhibitory nucleic acid, for example, a dsRNA, for example, an siRNA or shRNA; or for example, an inhibitory protein or system, for example, a clustered regularly interspaced short palindromic repeats (CRISPR), a transcription-activator like effector nuclease (TALEN), or a zinc finger endonuclease (ZFN), for example, as described herein, can be used to inhibit expression of a molecule that modulates or regulates, for example, inhibits, T-cell function in the CAR-expressing cell. In some embodiments the agent is an shRNA, for example, an shRNA described herein. In some embodiments, the agent that modulates or regulates, for example, inhibits, T-cell function is inhibited within a CAR-expressing cell. For example, a dsRNA molecule that inhibits expression of a molecule that modulates or regulates, for example, inhibits, T-cell function is linked to the nucleic acid that encodes a component, for example, all of the components, of the CAR.

In some embodiments, the agent which inhibits an inhibitory molecule comprises a first polypeptide, for example, an inhibitory molecule, associated with a second polypeptide that provides a positive signal to the cell, for example, an intracellular signaling domain described herein. In some embodiments, the agent comprises a first polypeptide, for example, of an inhibitory molecule such as PD1, PD-L1, CTLA4, TIM3, LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, or TGF beta, or a fragment of any of these (for example, at least a portion of an extracellular domain of any of these), and a second polypeptide which is an intracellular signaling domain described herein (for example, comprising a costimulatory domain (for example, 41BB, CD27 or CD28, for example, as described herein) and/or a primary signaling domain (for example, a CD3 zeta signaling domain described herein). In some embodiments, the agent comprises a first polypeptide of PD1 or a fragment thereof (for example, at least a portion of an extracellular domain of PD1), and a second polypeptide of an intracellular signaling domain described herein (for example, a CD28 signaling domain described herein and/or a CD3 zeta signaling domain described herein). PD1 is an inhibitory member of the CD28 family of receptors that also includes CD28, CTLA-4, ICOS, and BTLA. PD-1 is expressed on activated B cells, T cells and myeloid cells (Agata et al. 1996 Int. Immunol 8:765-75). Two ligands for PD1, PD-L1 and PD-L2 have been shown to downregulate T cell activation upon binding to PD1 (Freeman et a. 2000 J Exp Med 192:1027-34; Latchman et al. 2001 Nat Immunol 2:261-8; Carter et al. 2002 Eur J Immunol 32:634-43). PD-L1 is abundant in human cancers (Dong et al. 2003 J Mol Med 81:281-7; Blank et al. 2005 Cancer Immunol. Immunother 54:307-314; Konishi et al. 2004 Clin Cancer Res 10:5094). Immune suppression can be reversed by inhibiting the local interaction of PD1 with PD-L1.

In some embodiments, the agent comprises the extracellular domain (ECD) of an inhibitory molecule, for example, Programmed Death 1 (PD1), can be fused to a transmembrane domain and intracellular signaling domains such as 41BB and CD3 zeta (also referred to herein as a PD1 CAR). In some embodiments, the PD1 CAR, when used in combinations with an XCAR described herein, improves the persistence of the T cell. In some embodiments, the CAR is a PD1 CAR comprising the extracellular domain of PD1 indicated as underlined in SEQ ID NO: 24. In some embodiments, the PD1 CAR comprises the amino acid sequence of SEQ ID NO: 24.

In some embodiments, the PD1 CAR comprises the amino acid sequence of SEQ ID NO: 22.

In some embodiments, the agent comprises a nucleic acid sequence encoding the PD1 CAR, for example, the PD1 CAR described herein. In some embodiments, the nucleic acid sequence for the PD1 CAR is provided as SEQ ID NO: 23, with the PD1 ECD underlined.

In another example, in some embodiments, the agent which enhances the activity of a CAR-expressing cell can be a costimulatory molecule or costimulatory molecule ligand. Examples of costimulatory molecules include MHC class I molecule, BTLA and a Toll ligand receptor, as well as OX40, CD27, CD28, CDS, ICAM-1, LFA-1 (CD11a/CD18), ICOS (CD278), and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83, for example, as described herein. Examples of costimulatory molecule ligands include CD80, CD86, CD40L, ICOSL, CD70, OX40L, 4-1BBL, GITRL, and LIGHT. In embodiments, the costimulatory molecule ligand is a ligand for a costimulatory molecule different from the costimulatory molecule domain of the CAR. In embodiments, the costimulatory molecule ligand is a ligand for a costimulatory molecule that is the same as the costimulatory molecule domain of the CAR. In some embodiments, the costimulatory molecule ligand is 4-1BBL. In some embodiments, the costimulatory ligand is CD80 or CD86. In some embodiments, the costimulatory molecule ligand is CD70. In embodiments, a CAR-expressing immune effector cell described herein can be further engineered to express one or more additional costimulatory molecules or costimulatory molecule ligands.

Co-Expression of CAR with a Chemokine Receptor

In embodiments, the CAR-expressing cell described herein, for example, CD19 CAR-expressing cell, further comprises a chemokine receptor molecule. Transgenic expression of chemokine receptors CCR2b or CXCR2 in T cells enhances trafficking to CCL2- or CXCL1-secreting solid tumors including melanoma and neuroblastoma (Craddock et al., J Immunother. 2010 October; 33(8):780-8 and Kershaw et al., Hum Gene Ther. 2002 Nov. 1; 13(16):1971-80). Thus, without wishing to be bound by theory, it is believed that chemokine receptors expressed in CAR-expressing cells that recognize chemokines secreted by tumors, for example, solid tumors, can improve homing of the CAR-expressing cell to the tumor, facilitate the infiltration of the CAR-expressing cell to the tumor, and enhances antitumor efficacy of the CAR-expressing cell. The chemokine receptor molecule can comprise a naturally occurring or recombinant chemokine receptor or a chemokine-binding fragment thereof. A chemokine receptor molecule suitable for expression in a CAR-expressing cell (for example, CAR-Tx) described herein include a CXC chemokine receptor (for example, CXCR1, CXCR2, CXCR3, CXCR4, CXCR5, CXCR6, or CXCR7), a CC chemokine receptor (for example, CCR1, CCR2, CCR3, CCR4, CCR5, CCR6, CCR7, CCR8, CCR9, CCR10, or CCR11), a CX3C chemokine receptor (for example, CX3CR1), a XC chemokine receptor (for example, XCR1), or a chemokine-binding fragment thereof. In some embodiments, the chemokine receptor molecule to be expressed with a CAR described herein is selected based on the chemokine(s) secreted by the tumor. In some embodiments, the CAR-expressing cell described herein further comprises, for example, expresses, a CCR2b receptor or a CXCR2 receptor. In some embodiments, the CAR described herein and the chemokine receptor molecule are on the same vector or are on two different vectors. In embodiments where the CAR described herein and the chemokine receptor molecule are on the same vector, the CAR and the chemokine receptor molecule are each under control of two different promoters or are under the control of the same promoter.

### Nucleic Acid Constructs Encoding a CAR

The present invention also provides an immune effector cell, for example, made by a method described herein, that includes a nucleic acid molecule encoding one or more CAR constructs described herein. In some embodiments, the nucleic acid molecule is provided as a messenger RNA transcript. In some embodiments, the nucleic acid molecule is provided as a DNA construct.

The nucleic acid molecules described herein can be a DNA molecule, an RNA molecule, or a combination thereof. In some embodiments, the nucleic acid molecule is an mRNA encoding a CAR polypeptide as described herein. In other embodiments, the nucleic acid molecule is a vector that includes any of the aforesaid nucleic acid molecules.

In some embodiments, the antigen binding domain of a CAR of the invention (for example, a scFv) is encoded by a nucleic acid molecule whose sequence has been codon optimized for expression in a mammalian cell. In some embodiments, entire CAR construct of the invention is encoded by a nucleic acid molecule whose entire sequence has been codon optimized for expression in a mammalian cell. Codon optimization refers to the discovery that the frequency of occurrence of synonymous codons (i.e., codons that code for the same amino acid) in coding DNA is biased in different species. Such codon degeneracy allows an identical polypeptide to be encoded by a variety of nucleotide sequences. A variety of codon optimization methods is known in the art, and include, for example, methods disclosed in at least U.S. Pat. Nos. 5,786,464 and 6,114,148.

Accordingly, in some embodiments, an immune effector cell, for example, made by a method described herein, includes a nucleic acid molecule encoding a chimeric antigen receptor (CAR), wherein the CAR comprises an antigen binding domain that binds to a tumor antigen described herein, a transmembrane domain (for example, a transmembrane domain described herein), and an intracellular signaling domain (for example, an intracellular signaling domain described herein) comprising a stimulatory domain, for example, a costimulatory signaling domain (for example, a costimulatory signaling domain described herein) and/or a primary signaling domain (for example, a primary signaling domain described herein, for example, a zeta chain described herein).

The present invention also provides vectors in which a nucleic acid molecule encoding a CAR, for example, a nucleic acid molecule described herein, is inserted. Vectors derived from retroviruses such as the lentivirus are suitable tools to achieve long-term gene transfer since they allow long-term, stable integration of a transgene and its propagation in daughter cells. Lentiviral vectors have the added advantage over vectors derived from onco-retroviruses such as murine leukemia viruses in that they can transduce non-proliferating cells, such as hepatocytes. They also have the added advantage of low immunogenicity. A retroviral vector may also be, for example, a gammaretroviral vector. A gammaretroviral vector may include, for example, a promoter, a packaging signal (ψ), a primer binding site (PBS), one or more (for example, two) long terminal repeats (LTR), and a transgene of interest, for example, a gene encoding a CAR. A gammaretroviral vector may lack viral structural gens such as gag, pol, and env. Exemplary gammaretroviral vectors include Murine Leukemia Virus (MLV), Spleen-Focus Forming Virus (SFFV), and Myeloproliferative Sarcoma Virus (MPSV), and vectors derived therefrom. Other gammaretroviral vectors are described, for example, in Tobias Maetzig et al., “Gammaretroviral Vectors: Biology, Technology and Application” Viruses. 2011 June; 3(6): 677-713.

In some embodiments, the vector comprising the nucleic acid encoding the desired CAR is an adenoviral vector (A5/35). In some embodiments, the expression of nucleic acids encoding CARs can be accomplished using of transposons such as sleeping beauty, crisper, CAS9, and zinc finger nucleases. See below June et al. 2009 Nature Reviews Immunology 9.10: 704-716, is incorporated herein by reference.

In brief summary, the expression of natural or synthetic nucleic acids encoding CARs is typically achieved by operably linking a nucleic acid encoding the CAR polypeptide or portions thereof to a promoter and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

The nucleic acid can be cloned into a number of types of vectors. For example, the nucleic acid can be cloned into a vector including, but not limited to a plasmid, a phagemid, a phage derivative, an animal virus, and a cosmid. Vectors of particular interest include expression vectors, replication vectors, probe generation vectors, and sequencing vectors.

Further, the expression vector may be provided to a cell in the form of a viral vector. Viral vector technology is well known in the art and is described, for example, in Sambrook et al., 2012, MOLECULAR CLONING: A LABORATORY MANUAL, volumes 1-4, Cold Spring Harbor Press, NY), and in other virology and molecular biology manuals. Viruses, which are useful as vectors include, but are not limited to, retroviruses, adenoviruses, adeno-associated viruses, herpes viruses, and lentiviruses. In general, a suitable vector contains an origin of replication functional in at least one organism, a promoter sequence, convenient restriction endonuclease sites, and one or more selectable markers, (for example, WO 01/96584; WO 01/29058; and U.S. Pat. No. 6,326,193).

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In some embodiments, lentivirus vectors are used.

Additional promoter elements, for example, enhancers, regulate the frequency of transcriptional initiation. Typically, these are located in the region 30-110 bp upstream of the start site, although a number of promoters have been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline. Depending on the promoter, it appears that individual elements can function either cooperatively or independently to activate transcription. Exemplary promoters include the CMV IE gene, EF-1α, ubiquitin C, or phosphoglycerokinase (PGK) promoters.

An example of a promoter that is capable of expressing a CAR encoding nucleic acid molecule in a mammalian T cell is the EF1a promoter. The native EF1a promoter drives expression of the alpha subunit of the elongation factor-1 complex, which is responsible for the enzymatic delivery of aminoacyl tRNAs to the ribosome. The EF1a promoter has been extensively used in mammalian expression plasmids and has been shown to be effective in driving CAR expression from nucleic acid molecules cloned into a lentiviral vector. See, for example, Milone et al., Mol. Ther. 17(8): 1453-1464 (2009). In some embodiments, the EF1a promoter comprises the sequence provided in the Examples.

Another example of a promoter is the immediate early cytomegalovirus (CMV) promoter sequence. This promoter sequence is a strong constitutive promoter sequence capable of driving high levels of expression of any polynucleotide sequence operatively linked thereto. However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, an avian leukemia virus promoter, an Epstein-Barr virus immediate early promoter, a Rous sarcoma virus promoter, as well as human gene promoters such as, but not limited to, the actin promoter, the myosin promoter, the elongation factor-1α promoter, the hemoglobin promoter, and the creatine kinase promoter. Further, the invention should not be limited to the use of constitutive promoters. Inducible promoters are also contemplated as part of the invention. The use of an inducible promoter provides a molecular switch capable of turning on expression of the polynucleotide sequence which it is operatively linked when such expression is desired, or turning off the expression when expression is not desired. Examples of inducible promoters include, but are not limited to a metallothionine promoter, a glucocorticoid promoter, a progesterone promoter, and a tetracycline promoter.

Another example of a promoter is the phosphoglycerate kinase (PGK) promoter. In embodiments, a truncated PGK promoter (for example, a PGK promoter with one or more, for example, 1, 2, 5, 10, 100, 200, 300, or 400, nucleotide deletions when compared to the wild-type PGK promoter sequence) may be desired.

The nucleotide sequences of exemplary PGK promoters are provided below.

WT PGK Promoter:
(SEQ ID NO: 190)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCGCGGCGACGCAAAGGGCCTTGGTGCGGGT
CTCGTCGGCGCAGGGACGCGTTTGGGTCCCGACGGAACCTTTTCCGCGTT
GGGGTTGGGGCACCATAAGCT

### Exemplary Truncated PGK Promoters:

PGK100:
(SEQ ID NO: 198)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTG
PGK200:
(SEQ ID NO: 191)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACG
PGK300:
(SEQ ID NO: 192)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCG
PGK400:
(SEQ ID NO: 193)
ACCCCTCTCTCCAGCCACTAAGCCAGTTGCTCCCTCGGCTGACGGCTGCA
CGCGAGGCCTCCGAACGTCTTACGCCTTGTGGCGCGCCCGTCCTTGTCCC
GGGTGTGATGGCGGGGTGTGGGGCGGAGGGCGTGGCGGGGAAGGGCCGGC
GACGAGAGCCGCGCGGGACGACTCGTCGGCGATAACCGGTGTCGGGTAGC
GCCAGCCGCGCGACGGTAACGAGGGACCGCGACAGGCAGACGCTCCCATG
ATCACTCTGCACGCCGAAGGCAAATAGTGCAGGCCGTGCGGCGCTTGGCG
TTCCTTGGAAGGGCTGAATCCCCGCCTCGTCCTTCGCAGCGGCCCCCCGG
GTGTTCCCATCGCCGCTTCTAGGCCCACTGCGACGCTTGCCTGCACTTCT
TACACGCTCTGGGTCCCAGCCG

A vector may also include, for example, a signal sequence to facilitate secretion, a polyadenylation signal and transcription terminator (for example, from Bovine Growth Hormone (BGH) gene), an element allowing episomal replication and replication in prokaryotes (for example SV40 origin and ColE1 or others known in the art) and/or elements to allow selection (for example, ampicillin resistance gene and/or zeocin marker).

In order to assess the expression of a CAR polypeptide or portions thereof, the expression vector to be introduced into a cell can also contain either a selectable marker gene or a reporter gene or both to facilitate identification and selection of expressing cells from the population of cells sought to be transfected or infected through viral vectors. In some embodiments, the selectable marker may be carried on a separate piece of DNA and used in a co-transfection procedure. Both selectable markers and reporter genes may be flanked with appropriate regulatory sequences to enable expression in the host cells. Useful selectable markers include, for example, antibiotic-resistance genes, such as neo and the like.

Reporter genes are used for identifying potentially transfected cells and for evaluating the functionality of regulatory sequences. In general, a reporter gene is a gene that is not present in or expressed by the recipient organism or tissue and that encodes a polypeptide whose expression is manifested by some easily detectable property, for example, enzymatic activity. Expression of the reporter gene is assayed at a suitable time after the DNA has been introduced into the recipient cells. Suitable reporter genes may include genes encoding luciferase, beta-galactosidase, chloramphenicol acetyl transferase, secreted alkaline phosphatase, or the green fluorescent protein gene (for example, Ui-Tei et al., 2000 FEBS Letters 479: 79-82). Suitable expression systems are well known and may be prepared using known techniques or obtained commercially. In general, the construct with the minimal 5′ flanking region showing the highest level of expression of reporter gene is identified as the promoter. Such promoter regions may be linked to a reporter gene and used to evaluate agents for the ability to modulate promoter-driven transcription.

In embodiments, the vector may comprise two or more nucleic acid sequences encoding a CAR, for example, a CAR described herein, for example, a CD19 CAR, and a second CAR, for example, an inhibitory CAR or a CAR that specifically binds to an antigen other than CD19. In such embodiments, the two or more nucleic acid sequences encoding the CAR are encoded by a single nucleic molecule in the same frame and as a single polypeptide chain. In some embodiments, the two or more CARs, can, for example, be separated by one or more peptide cleavage sites. (for example, an auto-cleavage site or a substrate for an intracellular protease). Examples of peptide cleavage sites include T2A, P2A, E2A, or F2A sites.

Methods of introducing and expressing genes into a cell are known in the art. In the context of an expression vector, the vector can be readily introduced into a host cell, for example, mammalian, bacterial, yeast, or insect cell by any method, for example, one known in the art. For example, the expression vector can be transferred into a host cell by physical, chemical, or biological means.

Physical methods for introducing a polynucleotide into a host cell include calcium phosphate precipitation, lipofection, particle bombardment, microinjection, electroporation, and the like. Methods for producing cells comprising vectors and/or exogenous nucleic acids are well-known in the art. See, for example, Sambrook et al., 2012, MOLECULAR CLONING: A LABORATORY MANUAL, volumes 1-4, Cold Spring Harbor Press, NY). A suitable method for the introduction of a polynucleotide into a host cell is calcium phosphate transfection.

Biological methods for introducing a polynucleotide of interest into a host cell include the use of DNA and RNA vectors. Viral vectors, and especially retroviral vectors, have become the most widely used method for inserting genes into mammalian, for example, human cells. Other viral vectors can be derived from lentivirus, poxviruses, herpes simplex virus I, adenoviruses and adeno-associated viruses, and the like. See, for example, U.S. Pat. Nos. 5,350,674 and 5,585,362.

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (for example, an artificial membrane vesicle). Other methods of state-of-the-art targeted delivery of nucleic acids are available, such as delivery of polynucleotides with targeted nanoparticles or other suitable sub-micron sized delivery system.

In the case where a non-viral delivery system is utilized, an exemplary delivery vehicle is a liposome. The use of lipid formulations is contemplated for the introduction of the nucleic acids into a host cell (in vitro, ex vivo or in vivo). In some embodiments, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids are fatty substances which may be naturally occurring or synthetic lipids. For example, lipids include the fatty droplets that naturally occur in the cytoplasm as well as the class of compounds which contain long-chain aliphatic hydrocarbons and their derivatives, such as fatty acids, alcohols, amines, amino alcohols, and aldehydes.

Lipids suitable for use can be obtained from commercial sources. For example, dimyristyl phosphatidylcholine (“DMPC”) can be obtained from Sigma, St. Louis, MO; dicetyl phosphate (“DCP”) can be obtained from K & K Laboratories (Plainview, NY); cholesterol (“Choi”) can be obtained from Calbiochem-Behring; dimyristyl phosphatidylglycerol (“DMPG”) and other lipids may be obtained from Avanti Polar Lipids, Inc. (Birmingham, AL). Stock solutions of lipids in chloroform or chloroform/methanol can be stored at about −20° C. Chloroform is used as the only solvent since it is more readily evaporated than methanol. “Liposome” is a generic term encompassing a variety of single and multilamellar lipid vehicles formed by the generation of enclosed lipid bilayers or aggregates. Liposomes can be characterized as having vesicular structures with a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh et al., 1991 Glycobiology 5: 505-10). However, compositions that have different structures in solution than the normal vesicular structure are also encompassed. For example, the lipids may assume a micellar structure or merely exist as nonuniform aggregates of lipid molecules. Also contemplated are lipofectamine-nucleic acid complexes.

Regardless of the method used to introduce exogenous nucleic acids into a host cell or otherwise expose a cell to the inhibitor of the present invention, in order to confirm the presence of the recombinant nucleic acid sequence in the host cell, a variety of assays may be performed. Such assays include, for example, “molecular biological” assays well known to those of skill in the art, such as Southern and Northern blotting, RT-PCR and PCR; “biochemical” assays, such as detecting the presence or absence of a particular peptide, for example, by immunological means (ELISAs and Western blots) or by assays described herein to identify agents falling within the scope of the invention.

### RNA Transfection

Disclosed herein are methods for producing an in vitro transcribed RNA CAR. RNA CAR and methods of using the same are described, for example, in paragraphs 553-570 of in International Application WO2015/142675, filed Mar. 13, 2015, which is herein incorporated by reference in its entirety.

An immune effector cell can include a CAR encoded by a messenger RNA (mRNA). In some embodiments, the mRNA encoding a CAR described herein is introduced into an immune effector cell, for example, made by a method described herein, for production of a CAR-expressing cell.

In some embodiments, the in vitro transcribed RNA CAR can be introduced to a cell as a form of transient transfection. The RNA is produced by in vitro transcription using a polymerase chain reaction (PCR)-generated template. DNA of interest from any source can be directly converted by PCR into a template for in vitro mRNA synthesis using appropriate primers and RNA polymerase. The source of the DNA can be, for example, genomic DNA, plasmid DNA, phage DNA, cDNA, synthetic DNA sequence or any other appropriate source of DNA. The desired temple for in vitro transcription is a CAR described herein. For example, the template for the RNA CAR comprises an extracellular region comprising a single chain variable domain of an antibody to a tumor associated antigen described herein; a hinge region (for example, a hinge region described herein), a transmembrane domain (for example, a transmembrane domain described herein such as a transmembrane domain of CD8a); and a cytoplasmic region that includes an intracellular signaling domain, for example, an intracellular signaling domain described herein, for example, comprising the signaling domain of CD3-zeta and the signaling domain of 4-1BB.

In some embodiments, the DNA to be used for PCR contains an open reading frame. The DNA can be from a naturally occurring DNA sequence from the genome of an organism. In some embodiments, the nucleic acid can include some or all of the 5′ and/or 3′ untranslated regions (UTRs). The nucleic acid can include exons and introns. In some embodiments, the DNA to be used for PCR is a human nucleic acid sequence. In some embodiments, the DNA to be used for PCR is a human nucleic acid sequence including the 5′ and 3′ UTRs. The DNA can alternatively be an artificial DNA sequence that is not normally expressed in a naturally occurring organism. An exemplary artificial DNA sequence is one that contains portions of genes that are ligated together to form an open reading frame that encodes a fusion protein. The portions of DNA that are ligated together can be from a single organism or from more than one organism.

PCR is used to generate a template for in vitro transcription of mRNA which is used for transfection. Methods for performing PCR are well known in the art. Primers for use in PCR are designed to have regions that are substantially complementary to regions of the DNA to be used as a template for the PCR. “Substantially complementary,” as used herein, refers to sequences of nucleotides where a majority or all of the bases in the primer sequence are complementary, or one or more bases are non-complementary, or mismatched. Substantially complementary sequences are able to anneal or hybridize with the intended DNA target under annealing conditions used for PCR. The primers can be designed to be substantially complementary to any portion of the DNA template. For example, the primers can be designed to amplify the portion of a nucleic acid that is normally transcribed in cells (the open reading frame), including 5′ and 3′ UTRs. The primers can also be designed to amplify a portion of a nucleic acid that encodes a particular domain of interest. In some embodiments, the primers are designed to amplify the coding region of a human cDNA, including all or portions of the 5′ and 3′ UTRs. Primers useful for PCR can be generated by synthetic methods that are well known in the art. “Forward primers” are primers that contain a region of nucleotides that are substantially complementary to nucleotides on the DNA template that are upstream of the DNA sequence that is to be amplified. “Upstream” is used herein to refer to a location 5, to the DNA sequence to be amplified relative to the coding strand. “Reverse primers” are primers that contain a region of nucleotides that are substantially complementary to a double-stranded DNA template that are downstream of the DNA sequence that is to be amplified. “Downstream” is used herein to refer to a location 3′ to the DNA sequence to be amplified relative to the coding strand.

Any DNA polymerase useful for PCR can be used in the methods disclosed herein. The reagents and polymerase are commercially available from a number of sources.

Chemical structures with the ability to promote stability and/or translation efficiency may also be used. The RNA in embodiments has 5′ and 3′ UTRs. In some embodiments, the 5′ UTR is between one and 3000 nucleotides in length. The length of 5′ and 3′ UTR sequences to be added to the coding region can be altered by different methods, including, but not limited to, designing primers for PCR that anneal to different regions of the UTRs. Using this approach, one of ordinary skill in the art can modify the 5′ and 3′ UTR lengths required to achieve optimal translation efficiency following transfection of the transcribed RNA.

The 5′ and 3′ UTRs can be the naturally occurring, endogenous 5′ and 3′ UTRs for the nucleic acid of interest. Alternatively, UTR sequences that are not endogenous to the nucleic acid of interest can be added by incorporating the UTR sequences into the forward and reverse primers or by any other modifications of the template. The use of UTR sequences that are not endogenous to the nucleic acid of interest can be useful for modifying the stability and/or translation efficiency of the RNA. For example, it is known that AU-rich elements in 3′ UTR sequences can decrease the stability of mRNA. Therefore, 3′ UTRs can be selected or designed to increase the stability of the transcribed RNA based on properties of UTRs that are well known in the art.

In some embodiments, the 5′ UTR can contain the Kozak sequence of the endogenous nucleic acid. Alternatively, when a 5′ UTR that is not endogenous to the nucleic acid of interest is being added by PCR as described above, a consensus Kozak sequence can be redesigned by adding the 5′ UTR sequence. Kozak sequences can increase the efficiency of translation of some RNA transcripts, but does not appear to be required for all RNAs to enable efficient translation. The requirement for Kozak sequences for many mRNAs is known in the art. In other embodiments the 5′ UTR can be 5′UTR of an RNA virus whose RNA genome is stable in cells. In other embodiments various nucleotide analogues can be used in the 3′ or 5′ UTR to impede exonuclease degradation of the mRNA.

To enable synthesis of RNA from a DNA template without the need for gene cloning, a promoter of transcription should be attached to the DNA template upstream of the sequence to be transcribed. When a sequence that functions as a promoter for an RNA polymerase is added to the 5′ end of the forward primer, the RNA polymerase promoter becomes incorporated into the PCR product upstream of the open reading frame that is to be transcribed. In some embodiments, the promoter is a T7 polymerase promoter, as described elsewhere herein. Other useful promoters include, but are not limited to, T3 and SP6 RNA polymerase promoters. Consensus nucleotide sequences for T7, T3 and SP6 promoters are known in the art.

In some embodiments, the mRNA has both a cap on the 5′ end and a 3′ poly(A) tail which determine ribosome binding, initiation of translation and stability mRNA in the cell. On a circular DNA template, for instance, plasmid DNA, RNA polymerase produces a long concatameric product which is not suitable for expression in eukaryotic cells. The transcription of plasmid DNA linearized at the end of the 3′ UTR results in normal sized mRNA which is not effective in eukaryotic transfection even if it is polyadenylated after transcription.

On a linear DNA template, phage T7 RNA polymerase can extend the 3′ end of the transcript beyond the last base of the template (Schenborn and Mierendorf, Nuc Acids Res., 13:6223-36 (1985); Nacheva and Berzal-Herranz, Eur. J. Biochem., 270:1485-65 (2003).

The conventional method of integration of poly(A)/T stretches into a DNA template is molecular cloning. However, poly(A)/T sequence integrated into plasmid DNA can cause plasmid instability, which is why plasmid DNA templates obtained from bacterial cells are often highly contaminated with deletions and other aberrations. This makes cloning procedures not only laborious and time consuming but often not reliable. That is why a method which allows construction of DNA templates with poly(A)/T 3′ stretch without cloning highly desirable.

The poly(A)/T segment of the transcriptional DNA template can be produced during PCR by using a reverse primer containing a polyT tail, such as 100 T tail (SEQ ID NO: 31) (size can be 50-5000 T (SEQ ID NO: 32)), or after PCR by any other method, including, but not limited to, DNA ligation or in vitro recombination. Poly(A) tails also provide stability to RNAs and reduce their degradation. Generally, the length of a poly(A) tail positively correlates with the stability of the transcribed RNA. In some embodiments, the poly(A) tail is between 100 and 5000 adenosines (for example, SEQ ID NO: 33).

Poly(A) tails of RNAs can be further extended following in vitro transcription with the use of a poly(A) polymerase, such as E. coli poly(A) polymerase (E-PAP). In some embodiments, increasing the length of a poly(A) tail from 100 nucleotides to between 300 and 400 nucleotides (SEQ ID NO: 34) results in about a two-fold increase in the translation efficiency of the RNA. Additionally, the attachment of different chemical groups to the 3′ end can increase mRNA stability. Such attachment can contain modified/artificial nucleotides, aptamers and other compounds. For example, ATP analogs can be incorporated into the poly(A) tail using poly(A) polymerase. ATP analogs can further increase the stability of the RNA.

5′ caps on also provide stability to RNA molecules. In some embodiments, RNAs produced by the methods disclosed herein include a 5′ cap. The 5′ cap is provided using techniques known in the art and described herein (Cougot, et al., Trends in Biochem. Sci., 29:436-444 (2001); Stepinski, et al., RNA, 7:1468-95 (2001); Elango, et al., Biochim. Biophys. Res. Commun., 330:958-966 (2005)).

The RNAs produced by the methods disclosed herein can also contain an internal ribosome entry site (IRES) sequence. The IRES sequence may be any viral, chromosomal or artificially designed sequence which initiates cap-independent ribosome binding to mRNA and facilitates the initiation of translation. Any solutes suitable for cell electroporation, which can contain factors facilitating cellular permeability and viability such as sugars, peptides, lipids, proteins, antioxidants, and surfactants can be included.

RNA can be introduced into target cells using any of a number of different methods, for instance, commercially available methods which include, but are not limited to, electroporation (Amaxa Nucleofector-II (Amaxa Biosystems, Cologne, Germany)), (ECM 830 (BTX) (Harvard Instruments, Boston, Mass.) or the Gene Pulser II (BioRad, Denver, Colo.), Multiporator (Eppendort, Hamburg Germany), cationic liposome mediated transfection using lipofection, polymer encapsulation, peptide mediated transfection, or biolistic particle delivery systems such as “gene guns” (see, for example, Nishikawa, et al. Hum Gene Ther., 12(8):861-70 (2001).

### Non-Viral Delivery Methods

In some embodiments, non-viral methods can be used to deliver a nucleic acid encoding a CAR described herein into a cell or tissue or a subject.

In some embodiments, the non-viral method includes the use of a transposon (also called a transposable element). In some embodiments, a transposon is a piece of DNA that can insert itself at a location in a genome, for example, a piece of DNA that is capable of self-replicating and inserting its copy into a genome, or a piece of DNA that can be spliced out of a longer nucleic acid and inserted into another place in a genome. For example, a transposon comprises a DNA sequence made up of inverted repeats flanking genes for transposition.

Exemplary methods of nucleic acid delivery using a transposon include a Sleeping Beauty transposon system (SBTS) and a piggyBac™ (PB) transposon system. See, for example, Aronovich et al. Hum. Mol. Genet. 20.R1(2011):R14-20; Singh et al. Cancer Res. 15(2008):2961-2971; Huang et al. Mol. Ther. 16(2008):580-589; Grabundzija et al. Mol. Ther. 18(2010):1200-1209; Kebriaei et al. Blood. 122.21(2013):166; Williams. Molecular Therapy 16.9(2008):1515-16; Bell et al. Nat. Protoc. 2.12(2007):3153-65; and Ding et al. Cell. 122.3(2005):473-83, all of which are incorporated herein by reference.

The SBTS includes two components: 1) a transposon containing a transgene and 2) a source of transposase enzyme. The transposase can transpose the transposon from a carrier plasmid (or other donor DNA) to a target DNA, such as a host cell chromosome/genome. For example, the transposase binds to the carrier plasmid/donor DNA, cuts the transposon (including transgene(s)) out of the plasmid, and inserts it into the genome of the host cell. See, for example, Aronovich et al. supra.

Exemplary transposons include a pT2-based transposon. See, for example, Grabundzija et al. Nucleic Acids Res. 41.3(2013):1829-47; and Singh et al. Cancer Res. 68.8(2008): 2961-2971, all of which are incorporated herein by reference. Exemplary transposases include a Tc1/mariner-type transposase, for example, the SB10 transposase or the SB11 transposase (a hyperactive transposase which can be expressed, for example, from a cytomegalovirus promoter). See, for example, Aronovich et al.; Kebriaei et al.; and Grabundzija et al., all of which are incorporated herein by reference.

Use of the SBTS permits efficient integration and expression of a transgene, for example, a nucleic acid encoding a CAR described herein. Provided herein are methods of generating a cell, for example, T cell or NK cell, that stably expresses a CAR described herein, for example, using a transposon system such as SBTS.

In accordance with methods described herein, in some embodiments, one or more nucleic acids, for example, plasmids, containing the SBTS components are delivered to a cell (for example, T or NK cell). For example, the nucleic acid(s) are delivered by standard methods of nucleic acid (for example, plasmid DNA) delivery, for example, methods described herein, for example, electroporation, transfection, or lipofection. In some embodiments, the nucleic acid contains a transposon comprising a transgene, for example, a nucleic acid encoding a CAR described herein. In some embodiments, the nucleic acid contains a transposon comprising a transgene (for example, a nucleic acid encoding a CAR described herein) as well as a nucleic acid sequence encoding a transposase enzyme. In other embodiments, a system with two nucleic acids is provided, for example, a dual-plasmid system, for example, where a first plasmid contains a transposon comprising a transgene, and a second plasmid contains a nucleic acid sequence encoding a transposase enzyme. For example, the first and the second nucleic acids are co-delivered into a host cell.

In some embodiments, cells, for example, T or NK cells, are generated that express a CAR described herein by using a combination of gene insertion using the SBTS and genetic editing using a nuclease (for example, Zinc finger nucleases (ZFNs), Transcription Activator-Like Effector Nucleases (TALENs), the CRISPR/Cas system, or engineered meganuclease re-engineered homing endonucleases).

In some embodiments, use of a non-viral method of delivery permits reprogramming of cells, for example, T or NK cells, and direct infusion of the cells into a subject. Advantages of non-viral vectors include but are not limited to the ease and relatively low cost of producing sufficient amounts required to meet a patient population, stability during storage, and lack of immunogenicity.

### Methods of Manufacture/Production

The present invention also provides methods of making a cell disclosed herein, e.g., methods of engineering a T cell or NK cell to express a nucleic acid molecule encoding one or more CAR constructs described herein. In some embodiments, the manufacturing methods disclosed herein are used to manufacture a cell comprising a nucleic acid molecule encoding two CARs disclosed herein (e.g., an anti-BCMA CAR and an anti-CD19 CAR disclosed herein). In some embodiments, the manufacturing methods disclosed herein are used to manufacture a cell comprising a nucleic acid molecule encoding a diabody CAR disclosed herein, e.g., an anti-BCMA/anti-CD19 diabody CAR disclosed herein. In some embodiments, the manufacturing methods disclosed herein are used to manufacture a cell comprising two nucleic acid molecules, each of which encodes a CAR disclosed herein (e.g., one nucleic acid molecule encoding an anti-BCMA CAR and one nucleic acid molecule encoding an anti-CD19 CAR). In some embodiments, provided herein is a population of cells (for example, immune effector cells, for example, T cells or NK cells) made by any of the manufacturing processes described herein.

### Activation Process

In some embodiments, the methods disclosed herein may manufacture immune effector cells engineered to express one or more CARs in less than 24 hours. Without wishing to be bound by theory, the methods provided herein preserve the undifferentiated phenotype of T cells, such as naïve T cells, during the manufacturing process. These CAR-expressing cells with an undifferentiated phenotype may persist longer and/or expand better in vivo after infusion. In some embodiments, CART cells produced by the manufacturing methods provided herein comprise a higher percentage of stem cell memory T cells, compared to CART cells produced by the traditional manufacturing process, e.g., as measured using scRNA-seq (e.g., as measured using methods described in Example 7 with respect to FIG. 25A). In some embodiments, CART cells produced by the manufacturing methods provided herein comprise a higher percentage of effector T cells, compared to CART cells produced by the traditional manufacturing process, e.g., as measured using scRNA-seq (e.g., as measured using methods described in Example 7 with respect to FIG. 25B). In some embodiments, CART cells produced by the manufacturing methods provided herein better preserve the stemness of T cells, compared to CART cells produced by the traditional manufacturing process, e.g., as measured using scRNA-seq (e.g., as measured using methods described in Example 7 with respect to FIG. 25C). In some embodiments, CART cells produced by the manufacturing methods provided herein show a lower level of hypoxia, compared to CART cells produced by the traditional manufacturing process, e.g., as measured using scRNA-seq (e.g., as measured using methods described in Example 7 with respect to FIG. 25D). In some embodiments, CART cells produced by the manufacturing methods provided herein show a lower level of autophagy, compared to CART cells produced by the traditional manufacturing process, e.g., as measured using scRNA-seq (e.g., as measured using methods described in Example 7 with respect to FIG. 25E). In some embodiments, the immune effector cells are engineered to comprise a nucleic acid molecule encoding two CARs disclosed herein (e.g., an anti-BCMA CAR and an anti-CD19 CAR disclosed herein). In some embodiments, the immune effector cells are engineered to comprise a nucleic acid molecule encoding a diabody CAR disclosed herein, e.g., an anti-BCMA/anti-CD19 diabody CAR disclosed herein. In some embodiments, the immune effector cells are engineered to comprise two nucleic acid molecules, each of which encodes a CAR disclosed herein (e.g., one nucleic acid molecule encoding an anti-BCMA CAR and one nucleic acid molecule encoding an anti-CD19 CAR).

In some embodiments, the methods disclosed herein do not involve using a bead, such as Dynabeads® (for example, CD3/CD28 Dynabeads®), and do not involve a de-beading step. In some embodiments, the CART cells manufactured by the methods disclosed herein may be administered to a subject with minimal ex vivo expansion, for example, less than 1 day, less than 12 hours, less than 8 hours, less than 6 hours, less than 4 hours, less than 3 hours, less than 2 hours, less than 1 hour, or no ex vivo expansion. Accordingly, the methods described herein provide a fast manufacturing process of making improved CAR-expressing cell products for use in treating a disease in a subject.

In some embodiments, the present disclosure provides methods of making a population of cells (for example, T cells) that express a chimeric antigen receptor (CAR) (e.g., one or more CARs, e.g., two CARs) comprising: (i) contacting a population of cells (for example, T cells, for example, T cells isolated from a frozen or fresh leukapheresis product) with an agent that stimulates a CD3/TCR complex and/or an agent that stimulates a costimulatory molecule on the surface of the cells; (ii) contacting the population of cells (for example, T cells) with a nucleic acid molecule(s) (for example, a DNA or RNA molecule) encoding the CAR(s), thereby providing a population of cells (for example, T cells) comprising the nucleic acid molecule, and (iii) harvesting the population of cells (for example, T cells) for storage (for example, reformulating the population of cells in cryopreservation media) or administration, wherein: (a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 26 hours after the beginning of step (i), for example, no later than 22, 23, or 24 hours after the beginning of step (i), for example, no later than 24 hours after the beginning of step (i); (b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and step (iii) is performed no later than 30 hours after the beginning of step (ii), for example, no later than 22, 23, 24, 25, 26, 27, 28, 29, or 30 hours after the beginning of step (ii); or (c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i). In some embodiments, the nucleic acid molecule in step (ii) is a DNA molecule. In some embodiments, the nucleic acid molecule in step (ii) is an RNA molecule. In some embodiments, the nucleic acid molecule in step (ii) is on a viral vector, for example, a viral vector chosen from a lentivirus vector, an adenoviral vector, or a retrovirus vector. In some embodiments, the nucleic acid molecule in step (ii) is on a non-viral vector. In some embodiments, the nucleic acid molecule in step (ii) is on a plasmid. In some embodiments, the nucleic acid molecule in step (ii) is not on any vector. In some embodiments, step (ii) comprises transducing the population of cells (for example, T cells) a viral vector(s) comprising a nucleic acid molecule encoding the CAR(s).

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. Then the frozen apheresis sample is thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the activation process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the activation process described herein.

In some embodiments, cells (for example, T cells) are contacted with anti-CD3 and anti-CD28 antibodies for, for example, 12 hours, followed by transduction with a vector (for example, a lentiviral vector) (e.g. one or more vectors) encoding a CAR (e.g. one or more CARs). 24 hours after culture initiation, the cells are washed and formulated for storage or administration.

Without wishing to be bound by theory, brief CD3 and CD28 stimulation may promote efficient transduction of self-renewing T cells. Compared to traditional CART manufacturing approaches, the activation process provided herein does not involve prolonged ex vivo expansion. Similar to the cytokine process, the activation process provided herein also preserves undifferentiated T cells during CART manufacturing.

In some embodiments, the population of cells is contacted with an agent that stimulates a CD3/TCR complex and/or an agent that stimulates a costimulatory molecule on the surface of the cells.

In some embodiments, the agent that stimulates a CD3/TCR complex is an agent that stimulates CD3. In some embodiments, the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof. In some embodiments, the agent that stimulates a costimulatory molecule is an agent that stimulates CD28. In some embodiments, the agent that stimulates a CD3/TCR complex is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a CD3/TCR complex is an antibody. In some embodiments, the agent that stimulates a CD3/TCR complex is an anti-CD3 antibody. In some embodiments, the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand). In some embodiments, the agent that stimulates a costimulatory molecule is an antibody. In some embodiments, the agent that stimulates a costimulatory molecule is an anti-CD28 antibody. In some embodiments, the agent that stimulates a CD3/TCR complex or the agent that stimulates a costimulatory molecule does not comprise a bead. In some embodiments, the agent that stimulates a CD3/TCR complex comprises an anti-CD3 antibody covalently attached to a colloidal polymeric nanomatrix. In some embodiments, the agent that stimulates a costimulatory molecule comprises an anti-CD28 antibody covalently attached to a colloidal polymeric nanomatrix. In some embodiments, the agent that stimulates a CD3/TCR complex and the agent that stimulates a costimulatory molecule comprise T Cell TransAct™

In some embodiments, the matrix comprises or consists of a polymeric, for example, biodegradable or biocompatible inert material, for example, which is non-toxic to cells. In some embodiments, the matrix is composed of hydrophilic polymer chains, which obtain maximal mobility in aqueous solution due to hydration of the chains. In some embodiments, the mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum or alginate. Other polymers may include polyesters, polyethers, polyacrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. In some embodiments, the mobile matrix is a polymer of dextran.

In some embodiments, the population of cells is contacted with a nucleic acid molecule (e.g. one or more nucleic acid molecules) encoding a CAR (e.g. one or more CARs). In some embodiments, the population of cells is transduced with a DNA molecule (e.g. one or more DNA molecules) encoding a CAR (e.g. one or more CARs).

In some embodiments, in the case of a co-transduction of two nucleic acid molecules (e.g., lentiviral vectors), each of which encodes a CAR disclosed herein (e.g., one nucleic acid molecule encoding an anti-BCMA CAR and one nucleic acid molecule encoding an anti-CD19 CAR, as disclosed herein), each of the vectors containing nucleic acid molecules encoding the CAR can be added to the reaction mixture (e.g., containing a cell population) at a different multiplicity of infection (MOI).

Without wishing to be bound by theory, it is believed that, in some embodiments, using different MOIs for the vectors containing nucleic acid molecules which encode distinct CAR molecules may affect the final composition of the cellular population. For example, in the case of a co-transduction of a lentiviral vector encoding an anti-BCMA CAR and a lentiviral vector encoding an anti-CD19 CAR, different MOIs can be used to maximize the percent of mono BCMA CART cells and BCMA/CD19 dual CART cells, while resulting in fewer mono CD19 CART cells and untransduced cells.

In some embodiments, in the case of a co-transduction of a lentiviral vector encoding an anti-BCMA CAR and a lentiviral vector encoding an anti-CD19 CAR, a population of cells is contacted with the first viral vector at a multiplicity of infection (MOI) that is higher than, equal to, or less than an MOI at which the population of cells is contacted with the second viral vector. In some embodiments, the population of cells is contacted with the first viral vector at a multiplicity of infection (MOI) that is higher than an MOI at which the population of cells is contacted with the second viral vector.

In some embodiments, the population of cells is contacted with the first viral vector at a first MOI and with the second viral vector at a second MOI, such that a resultant population of cells comprises a first population of cells that comprise the anti-BCMA CAR but not the anti-CD19 CAR, a second population of cells that comprise the anti-CD19 CAR but not the anti-BCMA CAR, and a third population of cells that comprise both the anti-BCMA CAR and the anti-CD19 CAR, wherein:
(a) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10;
(b) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined, e.g., as determined by a method described in Example 10;
(c) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the resultant population, e.g., as determined by a method described in Example 10;
(d) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10; or
(e) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second population, e.g., as determined by a method described in Example 10.

In some embodiments, the population of cells is contacted with the second viral vector at an MOI (e.g., an MOI that is sufficiently lower than an MOI at which the population of cells is contacted with the first viral vector, such that in a resultant population of cells:
(a) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10;
(b) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined, e.g., as determined by a method described in Example 10;
(c) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the resultant population, e.g., as determined by a method described in Example 10;
(d) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10; or
(e) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second population, e.g., as determined by a method described in Example 10.

In some embodiments, the population of cells is contacted with the first viral vector at a first MOI, and the population of cells is contacted with the second viral vector at a second MOI, such that a resultant population of cells comprises:
(a) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10;
(b) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined, e.g., as determined by a method described in Example 10;
(c) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the resultant population, e.g., as determined by a method described in Example 10;
(d) the total number of viable cells in the second population is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined, e.g., as determined by a method described in Example 10; or
(e) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second population, e.g., as determined by a method described in Example 10.

In some embodiments, the population of cells is contacted with:
(a) the first viral vector at an MOI of about 1 to about 10 (e.g., about 2 to about 9, about 3 to about 8, about 4 to about 7, about 5 to about 6, about 1 to about 8, about 1 to about 6, about 1 to about 4, about 8 to about 10, about 6 to about 10, about 4 to about 10, about 1 to about 3, about 2 to about 4, about 3 to about 5, about 4 to about 6, about 5 to about 7, about 6 to about 8, about 7 to about to about 9, about 8 to about 10, about 2.5 to about 5, about 1, about 2, about 3, about 4, about 5, about 6, about 7, about 8, about 9, or about 10);
(b) the second viral vector at an MOI of about 0.1 to about 5 (e.g., about 0.2 to about 4, about 0.3 to about 3, about 0.4 to about 2, about 0.5 to about 1, about 0.6 to about 0.9, about 0.7 to about 0.8, about 0.1 to about 4, about 0.1 to about 3, about 0.1 to about 2, about 0.1 to about 1, about 0.1 to about 0.5, about 4 to about 5, about 3 to about 5, about 2 to about 5, about 1 to about 5, about 0.5 to about 5, about 0.2 to about 5, about 0.1 to about 0.5, about 0.2 to about 1, about 0.5 to about 2, about 1 to about 3, about 2 to about 4, about 3 to about 5, about 0.5 to about 1, about 0.1, about 0.2, about 0.3, about 0.4, about 0.5, about 0.6, about 0.7, about 0.8, about 0.9, about 1, about 2, about 3, about 4, or about 5);
(c) the first viral vector at an MOI that is at least about 10% (e.g., at least about 20%, 30%, 40%, 50%, 60%, 70%, 80%, or 90%) or at least about 1 fold (e.g., at least about 2, 3, 4, 5, 6, 7, 8, 9, 10, 15, 20, 25, 30, 35, 40, 50, 60, 70, 80, 90, or 100 fold, e.g., about 2 to about 50 fold, about 3 to 20 fold, about 5 to about 15 fold, or about 8 to about 10 fold) higher than an MOI at which the population of cells is contacted with the second viral vector; and/or
(d) the second viral vector at an MOI that is no more than 1/X, wherein X is 2, 3, 4, 5, 6, 7, 8, 9, 10, 12, 15, 20, 30, 40, 50, 60, 70. 80, 90, or 100, of an MOI at which the population of cells is contacted with the first viral vector.

In some embodiments, the population of cells is contacted with the first viral vector at an MOI of about 2.5 to about 5. In some embodiments, the population of cells is contacted with the second viral vector at an MOI of about 0.5 to about 1.0. In some embodiments, the first viral vector at an MOI that is about 8 to about 10 fold higher than an MOI at which the population of cells is contacted with the second viral vector. In some embodiments, the second viral vector at an MOI that is no more than 1/X, wherein X is 6, 8, 10, or 12, of an MOI at which the population of cells is contacted with the first viral vector.

In some embodiments, in step (ii), the population of cells is contacted with:
(a) the first viral vector at an MOI of between about 4 and about 5 (e.g., about 4.75); and/or
(b) the second viral vector at an MOI between about 0.2 and about 1 (e.g., about 0.5).

In some embodiments, in step (ii), the population of cells comprises about 1×10⁸ to about 5×10⁹ (e.g., about 2×10⁸ to about 2×10⁹ or about 4×10⁸ to about 1×10⁹ total viable cells. In some embodiments, the cells are suspended in a culture at a concentration of about 1×10⁶ to about 1×10⁷ (e.g., about 2×10⁶ to about 5×10⁶ or about 3×10⁶ to about 4×10⁶) viable cells/mL.

The precise MOI used for each vector can be adjusted or determined based on a number of factors, including, but not limited to, properties of the batch of viral vector, characteristics of the cells to be transduced, and transduction efficiency. In some embodiments, contacting the population of cells with the nucleic acid molecule(s) encoding the CAR(s) occurs simultaneously with contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 30, 29, 28, 27, 26, 25, 24, 23, 22, 21, 20, 19, 18, 17, 16, 15, 14, 13, 12, 11, 10, 9, 8, 7, 6, 5, 4, 3, 2, 1, or 0.5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 20 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 19 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 18 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 17 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 16 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 15 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 14 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 13 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 12 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 11 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 10 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 9 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 8 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 7 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 6 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 5 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 4 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 3 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule encoding the CAR(s) occurs no later than 2 hours after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 1 hour after the beginning of contacting the population of cells with the agent that stimulates a CD3/TCR complex and/or the agent that stimulates a costimulatory molecule on the surface of the cells described above. In some embodiments, contacting the population of cells with the nucleic acid molecule(s) encoding the CAR(s) occurs no later than 30 minutes after the beginning of contacting the population of cells with the agent that stimulates a CD3/TCR complex and/or the agent that stimulates a costimulatory molecule on the surface of the cells described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 26 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 25 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 24 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 23 hours after the beginning of contacting the population of cells with the agent that stimulates a CD3/TCR complex and/or the agent that stimulates a costimulatory molecule on the surface of the cells described above. In some embodiments, the population of cells is harvested for storage or administration no later than 22 hours after the beginning of contacting the population of cells with the agent that stimulates a CD3/TCR complex and/or the agent that stimulates a costimulatory molecule on the surface of the cells described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 5%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 10%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 15%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 20%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 25%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 30%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 35%, for example, as assessed by the number of living cells, compared to the population of cells before it is contacted with the agent that stimulates a CD3/TCR complex and/or the agent that stimulates a costimulatory molecule on the surface of the cells described above. In some embodiments, the population of cells is expanded by no more than 40%, for example, as assessed by the number of living cells, compared to the population of cells before it is contacted with the agent that stimulates a CD3/TCR complex and/or the agent that stimulates a costimulatory molecule on the surface of the cells described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the activation process is conducted in serum free cell media. In some embodiments, the activation process is conducted in cell media comprising one or more cytokines chosen from: IL-2, IL-15 (for example, hetIL-15 (IL15/sIL-15Ra)), or IL-6 (for example, IL-6/sIL-6Ra).

In some embodiments, hetIL-15 comprises the amino acid sequence of NWVNVISDLKKIEDLIQSMHIDATLYTESDVHPSCKVTAMKCFLLELQVISLESGDASIHDTVEN LIILANNSLSSNGNVTESGCKECEELEEKNIKEFLQSFVHIVQMFINTSITCPPPMSVEHADIWVK SYSLYSRERYICNSGFKRKAGTSSLTECVLNKATNVAHWTTPSLKCIRDPALVHQRPAPPSTVT TAGVTPQPESLSPSGKEPAASSPSSNNTAATTAAIVPGSQLMPSKSPSTGTTEISSHESSHGTPSQT TAKNWELTASASHQPPGVYPQG (SEQ ID NO: 309). In some embodiments, hetIL-15 comprises an amino acid sequence having at least about 70, 75, 80, 85, 90, 95, or 99% identity to SEQ ID NO: 309. In some embodiments, the activation process is conducted in cell media comprising a LSD1 inhibitor. In some embodiments, the activation process is conducted in cell media comprising a MALT1 inhibitor. In some embodiments, the serum free cell media comprises a serum replacement. In some embodiments, the serum replacement is CTS™ Immune Cell Serum Replacement (ICSR). In some embodiments, the level of ICSR can be, for example, up to 5%, for example, about 1%, 2%, 3%, 4%, or 5%. Without wishing to be bound by theory, using cell media, for example, Rapid Media shown in Table 21 or Table 25, comprising ICSR, for example, 2% ICSR, may improve cell viability during a manufacture process described herein.

In some embodiments, the present disclosure provides methods of making a population of cells (for example, T cells) that express a chimeric antigen receptor (CAR) comprising: (a) providing an apheresis sample (for example, a fresh or cryopreserved leukapheresis sample) collected from a subject; (b) selecting T cells from the apheresis sample (for example, using negative selection, positive selection, or selection without beads); (c) seeding isolated T cells at, for example, 1×10⁶ to 1×10⁷ cells/mL; (d) contacting T cells with an agent that stimulates T cells, for example, an agent that stimulates a CD3/TCR complex and/or an agent that stimulates a costimulatory molecule on the surface of the cells (for example, contacting T cells with anti-CD3 and/or anti-CD28 antibody, for example, contacting T cells with TransAct); (e) contacting T cells with a nucleic acid molecule(s) (for example, a DNA or RNA molecule) encoding the CAR(s) (for example, contacting T cells with a virus comprising a nucleic acid molecule(s) encoding the CAR(s)) for, for example, 6-48 hours, for example, 20-28 hours; and (f) washing and harvesting T cells for storage (for example, reformulating T cells in cryopreservation media) or administration. In some embodiments, step (f) is performed no later than 30 hours after the beginning of step (d) or (e), for example, no later than 22, 23, 24, 25, 26, 27, 28, 29, or 30 hours after the beginning of step (d) or (e).

In some embodiments, provided herein is a population of cells (for example, immune effector cells, for example, T cells or NK cells) made by any of the manufacturing processes described herein (e.g., the Activation Process described herein).

In some embodiments, the percentage of naïve cells, for example, naïve T cells, for example, CD45RA+CD45RO− CCR7+ T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15%, from, or (3) is increased, for example, by at least 5, 6, 7, 8, 9, 10, 11, 12, 13, 14, 15, 16, 17, 18, 19, 20, 21, 22, 23, 24, or 25%, as compared to, the percentage of naïve cells, for example, naïve T cells, for example, CD45RA+CD45RO− CCR7+ cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a higher percentage of naïve cells, for example, naïve T cells, for example, CD45RA+CD45RO− CCR7+ T cells (for example, at least 5, 6, 7, 8, 9, 10, 11, 12, 13, 14, 15, 16, 17, 18, 19, 20, 25, 30, 35, 40, 45, or 50% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of naïve cells, for example, naïve T cells, for example, CD45RA+CD45RO− CCR7+ T cells, in the population of cells at the end of the manufacturing process (for example, at the end of the cytokine process or the activation process described herein) is not less than 20, 25, 30, 35, 40, 45, 50, 55, or 60%.

In some embodiments, the percentage of central memory cells, for example, central memory T cells, for example, CD95+ central memory T cells, in the population of cells at the end of the manufacturing process (for example, at the end of the cytokine process or the activation process described herein) (1) is the same as, (2) differs, for example, by no more than 4, 5, 6, 7, 8, 9, 10, 11, 12, 13, 14, or 15% from, or (3) is decreased, for example, by at least 5, 6, 7, 8, 9, 10, 11, 12, 13, 14, 15, 16, 17, 18, 19, 20, 21, 22, 23, 24, or 25%, as compared to, the percentage of central memory cells, for example, central memory T cells, for example, CD95+ central memory T cells, in the population of cells at the beginning of the manufacturing process (for example, at the beginning of the cytokine process or the activation process described herein). In some embodiments, the population of cells at the end of the manufacturing process (for example, at the end of the cytokine process or the activation process described herein) shows a lower percentage of central memory cells, for example, central memory T cells, for example, CD95+ central memory T cells (for example, at least 5, 6, 7, 8, 9, 10, 11, 12, 13, 14, 15, 16, 17, 18, 19, 20, 25, 30, 35, 40, 45, or 50% lower), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the percentage of central memory cells, for example, central memory T cells, for example, CD95+ central memory T cells, in the population of cells at the end of the manufacturing process (for example, at the end of the cytokine process or the activation process described herein) is no more than 40, 45, 50, 55, 60, 65, 70, 75, or 80%.

In some embodiments, the population of cells at the end of the manufacturing process (for example, at the end of the cytokine process or the activation process described herein) after being administered in vivo, persists longer or expands at a higher level (for example, at least 20, 25, 30, 35, 40, 45, 50, 55, 60, 65, 70, 75, 80, 85, or 90% higher), compared with cells made by an otherwise similar method which lasts, for example, more than 26 hours (for example, which lasts more than 5, 6, 7, 8, 9, 10, 11, or 12 days) or which involves expanding the population of cells in vitro for, for example, more than 3 days (for example, expanding the population of cells in vitro for 3, 4, 5, 6, 7, 8, 9, 10, 11, 12, 13, 14, or 15 days).

In some embodiments, the population of cells has been enriched for IL6R-expressing cells (for example, cells that are positive for IL6Rα and/or IL6Rβ) prior to the beginning of the manufacturing process (for example, prior to the beginning of the cytokine process or the activation process described herein). In some embodiments, the population of cells comprises, for example, no less than 30, 35, 40, 45, 50, 55, 60, 65, 70, 75, or 80% of IL6R-expressing cells (for example, cells that are positive for IL6Rα and/or IL6Rβ) at the beginning of the manufacturing process (for example, at the beginning of the cytokine process or the activation process described herein).

### Cytokine Process

In some embodiments, the present disclosure provides methods of making a population of cells (for example, T cells) that express a chimeric antigen receptor (CAR) (e.g., one or more CARs, e.g., two CARs) comprising: (1) contacting a population of cells with a cytokine chosen from IL-2, IL-7, IL-15, IL-21, IL-6, or a combination thereof, (2) contacting the population of cells (for example, T cells) with a nucleic acid molecule(s) (for example, a DNA or RNA molecule) encoding the CAR(s), thereby providing a population of cells (for example, T cells) comprising the nucleic acid molecule, and (3) harvesting the population of cells (for example, T cells) for storage (for example, reformulating the population of cells in cryopreservation media) or administration, wherein: (a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, or 24 hours after the beginning of step (1), for example, no later than 24 hours after the beginning of step (1), or (b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1). In some embodiments, the nucleic acid molecule in step (2) is a DNA molecule. In some embodiments, the nucleic acid molecule in step (2) is an RNA molecule. In some embodiments, the nucleic acid molecule in step (2) is on a viral vector, for example, a viral vector chosen from a lentivirus vector, an adenoviral vector, or a retrovirus vector. In some embodiments, the nucleic acid molecule in step (2) is on a non-viral vector. In some embodiments, the nucleic acid molecule in step (2) is on a plasmid. In some embodiments, the nucleic acid molecule in step (2) is not on any vector. In some embodiments, step (2) comprises transducing the population of cells (for example, T cells) with a viral vector comprising a nucleic acid molecule(s) encoding the CAR(s). In some embodiments, the cells are engineered to comprise a nucleic acid molecule encoding two CARs disclosed herein (e.g., an anti-BCMA CAR and an anti-CD19 CAR disclosed herein). In some embodiments, the cells are engineered to comprise a nucleic acid molecule encoding a diabody CAR disclosed herein, e.g., an anti-BCMA/anti-CD19 diabody CAR disclosed herein. In some embodiments, the cells are engineered to comprise two nucleic acid molecules, each of which encodes a CAR disclosed herein (e.g., one nucleic acid molecule encoding an anti-BCMA CAR and one nucleic acid molecule encoding an anti-CD19 CAR).

In some embodiments, the population of cells (for example, T cells) is collected from an apheresis sample (for example, a leukapheresis sample) from a subject.

In some embodiments, the apheresis sample (for example, a leukapheresis sample) is collected from the subject and shipped as a frozen sample (for example, a cryopreserved sample) to a cell manufacturing facility. The frozen apheresis sample is then thawed, and T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the cytokine process described herein. In some embodiments, at the end of the cytokine process, the CAR T cells are cryopreserved and later thawed and administered to the subject. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject and shipped as a fresh product (for example, a product that is not frozen) to a cell manufacturing facility. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then seeded for CART manufacturing using the cytokine process described herein. In some embodiments, the selected T cells (for example, CD4+ T cells and/or CD8+ T cells) undergo one or more rounds of freeze-thaw before being seeded for CART manufacturing.

In some embodiments, the apheresis sample (for example, a leukapheresis sample) is collected from the subject. T cells (for example, CD4+ T cells and/or CD8+ T cells) are selected from the apheresis sample, for example, using a cell sorting machine (for example, a CliniMACS® Prodigy® device). The selected T cells (for example, CD4+ T cells and/or CD8+ T cells) are then shipped as a frozen sample (for example, a cryopreserved sample) to a cell manufacturing facility. The selected T cells (for example, CD4+ T cells and/or CD8+ T cells) are later thawed and seeded for CART manufacturing using the cytokine process described herein.

In some embodiments, after cells (for example, T cells) are seeded, one or more cytokines (for example, one or more cytokines chosen from IL-2, IL-7, IL-15 (for example, hetIL-15 (IL15/sIL-15Ra)), IL-21, or IL-6 (for example, IL-6/sIL-6R)) as well as a vector (for example, a lentiviral vector) (e.g. one or more vectors) encoding a CAR (e.g., one or more CARs) are added to the cells. After incubation for 20-24 hours, the cells are washed and formulated for storage or administration.

Different from traditional CART manufacturing approaches, the cytokine process provided herein does not involve CD3 and/or CD28 stimulation, or ex vivo T cell expansion. T cells that are contacted with anti-CD3 and anti-CD28 antibodies and expanded extensively ex vivo tend to show differentiation towards a central memory phenotype. Without wishing to be bound by theory, the cytokine process provided herein preserves or increases the undifferentiated phenotype of T cells during CART manufacturing, generating a CART product that may persist longer after being infused into a subject.

In some embodiments, the population of cells is contacted with one or more cytokines (for example, one or more cytokines chosen from IL-2, IL-7, IL-15 (for example, hetIL-15 (IL15/sIL-15Ra)), IL-21, or IL-6 (for example, IL-6/sIL-6Ra).

In some embodiments, the population of cells is contacted with IL-2. In some embodiments, the population of cells is contacted with IL-7. In some embodiments, the population of cells is contacted with IL-15 (for example, hetIL-15 (IL15/sIL-15Ra)). In some embodiments, the population of cells is contacted with IL-21. In some embodiments, the population of cells is contacted with IL-6 (for example, IL-6/sIL-6Ra). In some embodiments, the population of cells is contacted with IL-2 and IL-7. In some embodiments, the population of cells is contacted with IL-2 and IL-15 (for example, hetIL-15 (IL15/sIL-15Ra)). In some embodiments, the population of cells is contacted with IL-2 and IL-21. In some embodiments, the population of cells is contacted with IL-2 and IL-6 (for example, IL-6/sIL-6Ra). In some embodiments, the population of cells is contacted with IL-7 and IL-15 (for example, hetIL-15 (IL15/sIL-15Ra)). In some embodiments, the population of cells is contacted with IL-7 and IL-21. In some embodiments, the population of cells is contacted with IL-7 and IL-6 (for example, IL-6/sIL-6Ra). In some embodiments, the population of cells is contacted with IL-15 (for example, hetIL-15 (IL15/sIL-15Ra)) and IL-21. In some embodiments, the population of cells is contacted with IL-15 (for example, hetIL-15 (IL15/sIL-15Ra)) and IL-6 (for example, IL-6/sIL-6Ra). In some embodiments, the population of cells is contacted with IL-21 and IL-6 (for example, IL-6/sIL-6Ra). In some embodiments, the population of cells is contacted with IL-7, IL-15 (for example, hetIL-15 (IL15/sIL-15Ra)), and IL-21. In some embodiments, the population of cells is further contacted with a LSD1 inhibitor. In some embodiments, the population of cells is further contacted with a MALT1 inhibitor.

In some embodiments, the population of cells is contacted with 20, 30, 40, 50, 60, 70, 80, 90, 100, 110, 120, 130, 140, 150, 160, 170, 180, 190, 200, 210, 220, 230, 240, 250, 260, 270, 280, 290, or 300 U/ml of IL-2. In some embodiments, the population of cells is contacted with 1, 2, 3, 4, 5, 6, 7, 8, 9, 10, 11, 12, 13, 14, 15, 16, 17, 18, 19, or 20 ng/ml of IL-7. In some embodiments, the population of cells is contacted with 1, 2, 3, 4, 5, 6, 7, 8, 9, 10, 11, 12, 13, 14, 15, 16, 17, 18, 19, or 20 ng/ml of IL-15.

In some embodiments, the population of cells is contacted with a nucleic acid molecule (e.g. one or more nucleic acid molecules) encoding a CAR (e.g., one or more CARs). In some embodiments, the population of cells is transduced with a DNA molecule (e.g. one or more DNA molecules) encoding a CAR (e.g. one or more CARs).

In some embodiments, contacting the population of cells with the nucleic acid molecule encoding the CAR(s) occurs simultaneously with contacting the population of cells with the one or more cytokines described above. In some embodiments, contacting the population of cells with the nucleic acid molecule encoding the CAR(s) occurs no later than 1, 1.5, 2, 2.5, 3, 3.5, 4, 4.5, 5, 5.5, 6, 6.5, 7, 7.5, 8, 8.5, 9, 9.5 or 10 hours after the beginning of contacting the population of cells with the one or more cytokines described above. In some embodiments, contacting the population of cells with the nucleic acid molecule encoding the CAR(s) occurs no later than 5 hours after the beginning of contacting the population of cells with the one or more cytokines described above. In some embodiments, contacting the population of cells with the nucleic acid molecule encoding the CAR(s) occurs no later than 4 hours after the beginning of contacting the population of cells with the one or more cytokines described above. In some embodiments, contacting the population of cells with the nucleic acid molecule encoding the CAR(s) occurs no later than 3 hours after the beginning of contacting the population of cells with the one or more cytokines described above. In some embodiments, contacting the population of cells with the nucleic acid molecule encoding the CAR(s) occurs no later than 2 hours after the beginning of contacting the population of cells with the one or more cytokines described above. In some embodiments, contacting the population of cells with the nucleic acid molecule encoding the CAR(s) occurs no later than 1 hour after the beginning of contacting the population of cells with the one or more cytokines described above.

In some embodiments, the population of cells is harvested for storage or administration.

In some embodiments, the population of cells is harvested for storage or administration no later than 72, 60, 48, 36, 32, 31, 30, 29, 28, 27, 26, 25, 24, 23, 22, 21, 20, 19, or 18 hours after the beginning of contacting the population of cells with the one or more cytokines described above. In some embodiments, the population of cells is harvested for storage or administration no later than 26 hours after the beginning of contacting the population of cells with the one or more cytokines described above. In some embodiments, the population of cells is harvested for storage or administration no later than 25 hours after the beginning of contacting the population of cells with the one or more cytokines described above. In some embodiments, the population of cells is harvested for storage or administration no later than 24 hours after the beginning of contacting the population of cells with the one or more cytokines described above. In some embodiments, the population of cells is harvested for storage or administration no later than 23 hours after the beginning of contacting the population of cells with the one or more cytokines described above. In some embodiments, the population of cells is harvested for storage or administration no later than 22 hours after the beginning of contacting the population of cells with the one or more cytokines described above.

In some embodiments, the population of cells is not expanded ex vivo.

In some embodiments, the population of cells is expanded by no more than 5, 6, 7, 8, 9, 10, 11, 12, 13, 14, 15, 16, 17, 18, 19, 20, 25, 30, 35, 40, 45, 50, 55, or 60%, for example, as assessed by the number of living cells, compared to the population of cells before it is contacted with the one or more cytokines described above. In some embodiments, the population of cells is expanded by no more than 5%, for example, as assessed by the number of living cells, compared to the population of cells before it is contacted with the one or more cytokines described above. In some embodiments, the population of cells is expanded by no more than 10%, for example, as assessed by the number of living cells, compared to the population of cells before it is contacted with the one or more cytokines described above. In some embodiments, the population of cells is expanded by no more than 15%, for example, as assessed by the number of living cells, compared to the population of cells before it is contacted with the one or more cytokines described above. In some embodiments, the population of cells is expanded by no more than 20%, for example, as assessed by the number of living cells, compared to the population of cells before it is contacted with the one or more cytokines described above. In some embodiments, the population of cells is expanded by no more than 25%, for example, as assessed by the number of living cells, compared to the population of cells before it is contacted with the one or more cytokines described above. In some embodiments, the population of cells is expanded by no more than 30%, for example, as assessed by the number of living cells, compared to the population of cells before it is contacted with the one or more cytokines described above. In some embodiments, the population of cells is expanded by no more than 35%, for example, as assessed by the number of living cells, compared to the population of cells before it is contacted with the one or more cytokines described above. In some embodiments, the population of cells is expanded by no more than 40%, for example, as assessed by the number of living cells, compared to the population of cells before it is contacted with the one or more cytokines described above.

In some embodiments, the population of cells is expanded by no more than 1, 1.5, 2, 2.5, 3, 3.5, 4, 4.5, 5, 6, 7, 8, 9, 10, 11, 12, 16, 20, 24, 36, or 48 hours, for example, as assessed by the number of living cells, compared to the population of cells before it is contacted with the one or more cytokines described above.

In some embodiments, the population of cells is not contacted in vitro with an agent that stimulates a CD3/TCR complex (for example, an anti-CD3 antibody) and/or an agent that stimulates a costimulatory molecule on the surface of the cells (for example, an anti-CD28 antibody), or if contacted, the contacting step is less than 1, 1.5, 2, 2.5, 3, 3.5, 4, 4.5, or 5 hours.

In some embodiments, the population of cells is contacted in vitro with an agent that stimulates a CD3/TCR complex (for example, an anti-CD3 antibody) and/or an agent that stimulates a costimulatory molecule on the surface of the cells (for example, an anti-CD28 antibody) for 20, 21, 22, 23, 24, 25, 26, 27, or 28 hours.

In some embodiments, the population of cells manufactured using the cytokine process provided herein shows a higher percentage of naïve cells among CAR-expressing cells (for example, at least 5, 6, 7, 8, 9, 10, 11, 12, 13, 14, 15, 16, 17, 18, 19, 20, 25, 30, 35, 40, 45, 50, 55, or 60% higher), compared with cells made by an otherwise similar method which further comprises contacting the population of cells with, for example, an agent that binds a CD3/TCR complex (for example, an anti-CD3 antibody) and/or an agent that binds a costimulatory molecule on the surface of the cells (for example, an anti-CD28 antibody).

In some embodiments, the cytokine process provided herein is conducted in cell media comprising no more than 0, 0.5, 1, 1.5, 2, 2.5, 3, 3.5, 4, 4.5, 5, 5.5, 6, 6.5, 7, 7.5, or 8% serum. In some embodiments, the cytokine process provided herein is conducted in cell media comprising a LSD1 inhibitor, a MALT1 inhibitor, or a combination thereof.

### Additional Exemplary Manufacturing Methods

In some embodiments, cells, e.g., T cells or NK cells are activated, e.g., using anti-CD3/anti-CD28 antibody coated Dynabeads®, contacted with one or more nucleic acid molecules encoding a CAR (e.g. one or more CARs) and then expanded in vitro for, for example, 7, 8, 9, 10, or 11 days. In some embodiments, the cells, e.g., T cells or NK cells are selected from a fresh or cryopreserved leukapheresis sample, e.g., using positive or negative selection. In some embodiments, the cells are contacted with a nucleic acid molecule (e.g. one or more nucleic acid molecules) encoding a CAR (e.g. one or more CARs). In some embodiments, the cells are contacted with a nucleic acid molecule encoding two CARs disclosed herein (e.g., an anti-BCMA CAR and an anti-CD19 CAR). In some embodiments, the cells are contacted with two nucleic acid molecules, one expressing a first CAR (e.g., an anti-BCMA CAR) and the other expressing a second CAR (e.g., an anti-CD19 CAR). In some embodiments, the cells are contacted with a nucleic acid molecule encoding a diabody CAR (e.g., an anti-BCMA/anti-CD19 diabody CAR disclosed herein).

### Elutriation

In some embodiments, the methods described herein feature an elutriation method that removes unwanted cells, for example, monocytes and blasts, thereby resulting in an improved enrichment of desired immune effector cells suitable for CAR expression. In some embodiments, the elutriation method described herein is optimized for the enrichment of desired immune effector cells suitable for CAR expression from a previously frozen sample, for example, a thawed sample. In some embodiments, the elutriation method described herein provides a preparation of cells with improved purity as compared to a preparation of cells collected from the elutriation protocols known in the art. In some embodiments, the elutriation method described herein includes using an optimized viscosity of the starting sample, for example, cell sample, for example, thawed cell sample, by dilution with certain isotonic solutions (for example, PBS), and using an optimized combination of flow rates and collection volume for each fraction collected by an elutriation device. Exemplary elutriation methods that could be applied in the present invention are described on pages 48-51 of WO 2017/117112, herein incorporated by reference in its entirety.

### Density Gradient Centrifugation

Manufacturing of adoptive cell therapeutic product requires processing the desired cells, for example, immune effector cells, away from a complex mixture of blood cells and blood elements present in peripheral blood apheresis starting materials. Peripheral blood-derived lymphocyte samples have been successfully isolated using density gradient centrifugation through Ficoll solution. However, Ficoll is not a preferred reagent for isolating cells for therapeutic use, as Ficoll is not qualified for clinical use. In addition, Ficoll contains glycol, which has toxic potential to the cells. Furthermore, Ficoll density gradient centrifugation of thawed apheresis products after cryopreservation yields a suboptimal T cell product, for example, as described in the Examples herein. For example, a loss of T cells in the final product, with a relative gain of non-T cells, especially undesirable B cells, blast cells and monocytes was observed in cell preparations isolated by density gradient centrifugation through Ficoll solution.

Without wishing to be bound by theory, it is believed that immune effector cells, for example, T cells, dehydrate during cryopreservation to become denser than fresh cells. Without wishing to be bound by theory, it is also believed that immune effector cells, for example, T cells, remain denser longer than the other blood cells, and thus are more readily lost during Ficoll density gradient separation as compared to other cells. Accordingly, without wishing to be bound by theory, a medium with a density greater than Ficoll is believed to provide improved isolation of desired immune effector cells in comparison to Ficoll or other mediums with the same density as Ficoll, for example, 1.077 g/mL.

In some embodiments, the density gradient centrifugation method described herein includes the use of a density gradient medium comprising iodixanol. In some embodiments, the density gradient medium comprises about 60% iodixanol in water.

In some embodiments, the density gradient centrifugation method described herein includes the use of a density gradient medium having a density greater than Ficoll. In some embodiments, the density gradient centrifugation method described herein includes the use of a density gradient medium having a density greater than 1.077 g/mL, for example, greater than 1.077 g/mL, greater than 1.1 g/mL, greater than 1.15 g/mL, greater than 1.2 g/mL, greater than 1.25 g/mL, greater than 1.3 g/mL, greater than 1.31 g/mL. In some embodiments, the density gradient medium has a density of about 1.32 g/mL.

Additional embodiments of density gradient centrifugation are described on pages 51-53 of WO 2017/117112, herein incorporated by reference in its entirety.

### Enrichment by Selection

Provided herein are methods for selection of specific cells to improve the enrichment of the desired immune effector cells suitable for CAR expression. In some embodiments, the selection comprises a positive selection, for example, selection for the desired immune effector cells. In some embodiments, the selection comprises a negative selection, for example, selection for unwanted cells, for example, removal of unwanted cells. In embodiments, the positive or negative selection methods described herein are performed under flow conditions, for example, by using a flow-through device, for example, a flow-through device described herein. Exemplary positive and negative selections are described on pages 53-57 of WO 2017/117112, herein incorporated by reference in its entirety. Selection methods can be performed under flow conditions, for example, by using a flow-through device, also referred to as a cell processing system, to further enrich a preparation of cells for desired immune effector cells, for example, T cells, suitable for CAR expression. Exemplary flow-through devices are described on pages 57-70 of WO 2017/117112, herein incorporated by reference in its entirety. Exemplary cell separation and debeading methods are described on pages 70-78 of WO 2017/117112, herein incorporated by reference in its entirety.

Selection procedures are not limited to ones described on pages 57-70 of WO 2017/117112. Negative T cell selection via removal of unwanted cells with CD19, CD14 and CD26 Miltenyi beads in combination with column technology (CliniMACS® Plus or CliniMACS® Prodigy®) or positive T cell selection with a combination of CD4 and CD8 Miltenyi beads and column technology (CliniMACS® Plus or CliniMACS® Prodigy®) can be used. Alternatively, column-free technology with releasable CD3 beads (GE Healthcare) can be used.

In addition, bead-free technologies such as ThermoGenesis X-series devices can be utilized as well.

### Clinical Applications

All of the processes herein may be conducted according to clinical good manufacturing practice (cGMP) standards.

The processes may be used for cell purification, enrichment, harvesting, washing, concentration or for cell media exchange, particularly during the collection of raw, starting materials (particularly cells) at the start of the manufacturing process, as well as during the manufacturing process for the selection or expansion of cells for cell therapy.

The cells may include any plurality of cells. The cells may be of the same cell type, or mixed cell types. In addition, the cells may be from one donor, such as an autologous donor or a single allogenic donor for cell therapy. The cells may be obtained from patients by, for example, leukapheresis or apheresis. The cells may include T cells, for example may include a population that has greater than 50% T cells, greater than 60% T cells, greater than 70% T cells, greater than 80% T cells, or 90% T cells.

Selection processes may be particularly useful in selecting cells prior to culture and expansion. For instance, paramagnetic particles coated with anti-CD3 and/or anti CD28 may be used to select T cells for expansion or for introduction of a nucleic acid encoding a chimeric antigen receptor (CAR) or other protein. Such a process is used to produce CTL019 T cells for treatment of acute lymphoblastic leukemia (ALL).

The debeading processes and modules disclosed herein may be particularly useful in the manufacture of cells for cell therapy, for example in purifying cells prior to, or after, culture and expansion. For instance, paramagnetic particles coated with anti-CD3 and/or anti CD28 antibodies may be used to selectively expand T cells, for example T cells that are, or will be, modified by introduction of a nucleic acid encoding a chimeric antigen receptor (CAR) or other protein, such that the CAR is expressed by the T cells. During the manufacture of such T cells, the debeading processes or modules may be used to separate T cells from the paramagnetic particles. Such a debeading process or module is used to produce, for example, CTL019 T cells for treatment of acute lymphoblastic leukemia (ALL).

In one such process, illustrated here by way of example, cells, for example, T cells, are collected from a donor (for example, a patient to be treated with an autologous chimeric antigen receptor T cell product) via apheresis (for example, leukapheresis). Collected cells may then be optionally purified, for example, by an elutriation step, or via positive or negative selection of target cells (for example, T cells). Paramagnetic particles, for example, anti-CD3/anti-CD28-coated paramagnetic particles, may then be added to the cell population, to expand the T cells. The process may also include a transduction step, wherein nucleic acid encoding one or more desired proteins, for example, a CAR, for example a CAR targeting CD19, is introduced into the cell. The nucleic acid may be introduced in a lentiviral vector. The cells, for example, the lentivirally transduced cells, may then be expanded for a period of days, for example, 1, 2, 3, 4, 5, 6, 7, 8, 9, 10, or more days, for example in the presence of a suitable medium. After expansion, the debeading processes/modules disclosed herein may be used to separate the desired T cells from the paramagnetic particles. The process may include one or more debeading steps according to the processes of the present disclosure. The debeaded cells may then be formulated for administration to the patient. Examples of CAR T cells and their manufacture are further described, for example, in WO2012/079000, which is incorporated herein by reference in its entirety. The systems and methods of the present disclosure may be used for any cell separation/purification/debeading processes described in or associated with WO2012/079000. Additional CAR T manufacturing processes are described in, for example, WO2016109410 and WO2017117112, herein incorporated by reference in their entireties.

The systems and methods herein may similarly benefit other cell therapy products by wasting fewer desirable cells, causing less cell trauma, and more reliably removing magnetic and any non-paramagnetic particles from cells with less or no exposure to chemical agents, as compared to conventional systems and methods.

Although only exemplary embodiments of the disclosure are specifically described above, it will be appreciated that modifications and variations of these examples are possible without departing from the spirit and intended scope of the disclosure. For example, the magnetic modules and systems containing them may be arranged and used in a variety of configurations in addition to those described. Besides, non-magnetic modules can be utilized as well. In addition, the systems and methods may include additional components and steps not specifically described herein. For instance, methods may include priming, where a fluid is first introduced into a component to remove bubbles and reduce resistance to cell suspension or buffer movement. Furthermore, embodiments may include only a portion of the systems described herein for use with the methods described herein. For example, embodiments may relate to disposable modules, hoses, etc. usable within non-disposable equipment to form a complete system able to separate or debead cells to produce a cell product.

Additional manufacturing methods and processes that can be combined with the present invention have been described in the art. For example, pages 86-91 of WO 2017/117112 describe improved wash steps and improved manufacturing process.

### Sources of Immune Effector Cells

This section provides additional methods or steps for obtaining an input sample comprising desired immune effector cells, isolating and processing desired immune effector cells, for example, T cells, and removing unwanted materials, for example, unwanted cells. The additional methods or steps described in this section can be used in combination with any of the elutriation, density gradient centrifugation, selection under flow conditions, or improved wash step described in the preceding sections.

A source of cells, for example, T cells or natural killer (NK) cells, can be obtained from a subject. Examples of subjects include humans, monkeys, chimpanzees, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In some embodiments of the present disclosure, immune effector cells, for example, T cells, can be obtained from a unit of blood collected from a subject using any number of techniques known to the skilled artisan, and any of the methods disclosed herein, in any combination of steps thereof. In some embodiments, cells from the circulating blood of an individual are obtained by apheresis. The apheresis product typically contains lymphocytes, including T cells, monocytes, granulocytes, B cells, other nucleated white blood cells, red blood cells, and platelets. In some embodiments, the cells collected by apheresis may be washed to remove the plasma fraction and, optionally, to place the cells in an appropriate buffer or media for subsequent processing steps. In some embodiments, the cells are washed with phosphate buffered saline (PBS). In some embodiments, the wash solution lacks calcium and may lack magnesium or may lack many if not all divalent cations. In some embodiments, the cells are washed using the improved wash step described herein.

Initial activation steps in the absence of calcium can lead to magnified activation. As those of ordinary skill in the art would readily appreciate a washing step may be accomplished by methods known to those in the art, such as by using a semi-automated “flow-through” centrifuge (for example, the Cobe 2991 cell processor, the Baxter CytoMate™, or the Haemonetics Cell Saver 5), Haemonetics Cell Saver Elite (GE Healthcare Sepax or Sefia), or a device utilizing the spinning membrane filtration technology (Fresenius Kabi LOVO), according to the manufacturer's instructions. After washing, the cells may be resuspended in a variety of biocompatible buffers, such as, for example, Ca-free, Mg-free PBS, PlasmaLyte A, PBS-EDTA supplemented with human serum albumin (HSA), or other saline solution with or without buffer. Alternatively, the undesirable components of the apheresis sample may be removed and the cells directly resuspended in culture media.

In some embodiments, desired immune effector cells, for example, T cells, are isolated from peripheral blood lymphocytes by lysing the red blood cells and depleting the monocytes, for example, by centrifugation through a PERCOLL™ gradient or by counterflow centrifugal elutriation.

The methods described herein can include, for example, selection of a specific subpopulation of immune effector cells, for example, T cells, that are a T regulatory cell-depleted population, for example, CD25+ depleted cells or CD25^{high} depleted cells, using, for example, a negative selection technique, for example, described herein. In some embodiments, the population of T regulatory-depleted cells contains less than 30%, 25%, 20%, 15%, 10%, 5%, 4%, 3%, 2%, 1% of CD25+ cells or CD25 high cells.

In some embodiments, T regulatory cells, for example, CD25+ T cells or CD25^{high} T cells, are removed from the population using an anti-CD25 antibody, or fragment thereof, or a CD25-binding ligand, for example IL-2. In some embodiments, the anti-CD25 antibody, or fragment thereof, or CD25-binding ligand is conjugated to a substrate, for example, a bead, or is otherwise coated on a substrate, for example, a bead. In some embodiments, the anti-CD25 antibody, or fragment thereof, is conjugated to a substrate as described herein.

In some embodiments, the T regulatory cells, for example, CD25+ T cells or CD25^{high} T cells, are removed from the population using CD25 depleting reagent from Miltenyi™. In some embodiments, the ratio of cells to CD25 depletion reagent is 1 e7 cells to 20 μL, or 1 e7 cells to 15 μL, or 1 e7 cells to 10 μL, or 1 e7 cells to 5 μL, or 1 e7 cells to 2.5 μL, or 1 e7 cells to 1.25 μL. In some embodiments, for example, for T regulatory cells, greater than 500 million cells/ml is used. In some embodiments, a concentration of cells of 600, 700, 800, or 900 million cells/ml is used.

In some embodiments, the population of immune effector cells to be depleted includes about 6×10⁹ CD25+ T cells. In some embodiments, the population of immune effector cells to be depleted include about 1×10⁹ to 1×10¹⁰ CD25+ T cell, and any integer value in between. In some embodiments, the resulting population T regulatory-depleted cells has 2×10⁹ T regulatory cells, for example, CD25+ cells or CD25^{high} cells, or less (for example, 1×10⁹, 5×10⁸, 1×10⁸, 5×10⁷, 1×10⁷, or less T regulatory cells).

In some embodiments, the T regulatory cells, for example, CD25+ cells or CD25^{high} cells, are removed from the population using the CliniMAC system with a depletion tubing set, such as, for example, tubing 162-01. In some embodiments, the CliniMAC system is run on a depletion setting such as, for example, DEPLETION2.1.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product significantly reduces the risk of subject relapse. For example, methods of depleting Treg cells are known in the art. Methods of decreasing Treg cells include, but are not limited to, cyclophosphamide, anti-GITR antibody (an anti-GITR antibody described herein), CD25-depletion, and combinations thereof.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

Without wishing to be bound by a particular theory, decreasing the level of negative regulators of immune cells (for example, decreasing the number of unwanted immune cells, for example, Treg cells), in a subject prior to apheresis or during manufacturing of a CAR-expressing cell product can reduce the risk of a subject's relapse. In some embodiments, a subject is pre-treated with one or more therapies that reduce Treg cells prior to collection of cells for CAR-expressing cell product manufacturing, thereby reducing the risk of subject relapse to CAR-expressing cell treatment. In some embodiments, methods of decreasing Treg cells include, but are not limited to, administration to the subject of one or more of cyclophosphamide, anti-GITR antibody, CD25-depletion, or a combination thereof. In some embodiments, methods of decreasing Treg cells include, but are not limited to, administration to the subject of one or more of cyclophosphamide, anti-GITR antibody, CD25-depletion, or a combination thereof. Administration of one or more of cyclophosphamide, anti-GITR antibody, CD25-depletion, or a combination thereof, can occur before, during or after an infusion of the CAR-expressing cell product. Administration of one or more of cyclophosphamide, anti-GITR antibody, CD25-depletion, or a combination thereof, can occur before, during or after an infusion of the CAR-expressing cell product.

In some embodiments, the manufacturing methods comprise reducing the number of (for example, depleting) Treg cells prior to manufacturing of the CAR-expressing cell. For example, manufacturing methods comprise contacting the sample, for example, the apheresis sample, with an anti-GITR antibody and/or an anti-CD25 antibody (or fragment thereof, or a CD25-binding ligand), for example, to deplete Treg cells prior to manufacturing of the CAR-expressing cell (for example, T cell, NK cell) product.

In some embodiments, a subject is pre-treated with cyclophosphamide prior to collection of cells for CAR-expressing cell product manufacturing, thereby reducing the risk of subject relapse to CAR-expressing cell treatment (for example, CTL019 treatment). In some embodiments, a subject is pre-treated with an anti-GITR antibody prior to collection of cells for CAR-expressing cell (for example, T cell or NK cell) product manufacturing, thereby reducing the risk of subject relapse to CAR-expressing cell treatment.

In some embodiments, the CAR-expressing cell (for example, T cell, NK cell) manufacturing process is modified to deplete Treg cells prior to manufacturing of the CAR-expressing cell (for example, T cell, NK cell) product (for example, a CTL019 product). In some embodiments, CD25-depletion is used to deplete Treg cells prior to manufacturing of the CAR-expressing cell (for example, T cell, NK cell) product (for example, a CTL019 product).

In some embodiments, the population of cells to be removed are neither the regulatory T cells or tumor cells, but cells that otherwise negatively affect the expansion and/or function of CART cells, for example cells expressing CD14, CD11b, CD33, CD15, or other markers expressed by potentially immune suppressive cells. In some embodiments, such cells are envisioned to be removed concurrently with regulatory T cells and/or tumor cells, or following said depletion, or in another order.

The methods described herein can include more than one selection step, for example, more than one depletion step. Enrichment of a T cell population by negative selection can be accomplished, for example, with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail can include antibodies to CD14, CD20, CD11b, CD16, HLA-DR, and CD8.

The methods described herein can further include removing cells from the population which express a tumor antigen, for example, a tumor antigen that does not comprise CD25, for example, CD19, CD30, CD38, CD123, CD20, CD14 or CD11b, to thereby provide a population of T regulatory-depleted, for example, CD25+ depleted or CD25^{high} depleted, and tumor antigen depleted cells that are suitable for expression of a CAR, for example, a CAR described herein. In some embodiments, tumor antigen expressing cells are removed simultaneously with the T regulatory, for example, CD25+ cells or CD25^{high} cells. For example, an anti-CD25 antibody, or fragment thereof, and an anti-tumor antigen antibody, or fragment thereof, can be attached to the same substrate, for example, bead, which can be used to remove the cells or an anti-CD25 antibody, or fragment thereof, or the anti-tumor antigen antibody, or fragment thereof, can be attached to separate beads, a mixture of which can be used to remove the cells. In other embodiments, the removal of T regulatory cells, for example, CD25+ cells or CD25^{high} cells, and the removal of the tumor antigen expressing cells is sequential, and can occur, for example, in either order.

Also provided are methods that include removing cells from the population which express a check point inhibitor, for example, a check point inhibitor described herein, for example, one or more of PD1+ cells, LAG3+ cells, and TIM3+ cells, to thereby provide a population of T regulatory-depleted, for example, CD25+ depleted cells, and check point inhibitor depleted cells, for example, PD1+, LAG3+ and/or TIM3+ depleted cells. Exemplary check point inhibitors include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta), for example, as described herein. In some embodiments, check point inhibitor expressing cells are removed simultaneously with the T regulatory, for example, CD25+ cells or CD25^{high} cells. For example, an anti-CD25 antibody, or fragment thereof, and an anti-check point inhibitor antibody, or fragment thereof, can be attached to the same bead which can be used to remove the cells, or an anti-CD25 antibody, or fragment thereof, and the anti-check point inhibitor antibody, or fragment there, can be attached to separate beads, a mixture of which can be used to remove the cells.

In other embodiments, the removal of T regulatory cells, for example, CD25+ cells or CD25^{high} cells, and the removal of the check point inhibitor expressing cells is sequential, and can occur, for example, in either order.

Methods described herein can include a positive selection step. For example, T cells can isolated by incubation with anti-CD3/anti-CD28 (for example, 3×28)-conjugated beads, such as Dynabeads® M-450 CD3/CD28 T, for a time period sufficient for positive selection of the desired T cells. In some embodiments, the time period is about 30 minutes. In some embodiments, the time period ranges from 30 minutes to 36 hours or longer and all integer values there between. In some embodiments, the time period is at least 1, 2, 3, 4, 5, or 6 hours. In some embodiments, the time period is 10 to 24 hours, for example, 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points.

In some embodiments, a T cell population can be selected that expresses one or more of IFN-γ, TNFα, IL-17A, IL-2, IL-3, IL-4, GM-CSF, IL-10, IL-13, granzyme B, and perforin, or other appropriate molecules, for example, other cytokines. Methods for screening for cell expression can be determined, for example, by the methods described in PCT Publication No.: WO 2013/126712.

For isolation of a desired population of cells by positive or negative selection, the concentration of cells and surface (for example, particles such as beads) can be varied. In some embodiments, it may be desirable to significantly decrease the volume in which beads and cells are mixed together (for example, increase the concentration of cells), to ensure maximum contact of cells and beads. For example, in some embodiments, a concentration of 10 billion cells/ml, 9 billion/ml, 8 billion/ml, 7 billion/ml, 6 billion/ml, or 5 billion/ml is used. In some embodiments, a concentration of 1 billion cells/ml is used. In some embodiments, a concentration of cells from 75, 80, 85, 90, 95, or 100 million cells/ml is used. In some embodiments, concentrations of 125 or 150 million cells/ml can be used.

Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (for example, leukemic blood, tumor tissue, etc.). Such populations of cells may have therapeutic value and would be desirable to obtain. For example, using high concentration of cells allows more efficient selection of CD8+ T cells that normally have weaker CD28 expression.

In some embodiments, it may be desirable to use lower concentrations of cells. By significantly diluting the mixture of T cells and surface (for example, particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In some embodiments, the concentration of cells used is 5×10⁶/ml. In some embodiments, the concentration used can be from about 1×10⁵/ml to 1×10⁶/ml, and any integer value in between.

In some embodiments, the cells may be incubated on a rotator for varying lengths of time at varying speeds at either 2-10° C. or at room temperature.

In some embodiments, a plurality of the immune effector cells of the population do not express diglycerol kinase (DGK), for example, is DGK-deficient. In some embodiments, a plurality of the immune effector cells of the population do not express Ikaros, for example, is Ikaros-deficient. In some embodiments, a plurality of the immune effector cells of the population do not express DGK and Ikaros, for example, is both DGK and Ikaros-deficient.

T cells for stimulation can also be frozen after a washing step. Wishing not to be bound by theory, the freeze and subsequent thaw step provides a more uniform product by removing granulocytes and to some extent monocytes in the cell population. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

In some embodiments, cryopreserved cells are thawed and washed as described herein and allowed to rest for one hour at room temperature prior to activation using the methods of the present invention.

Also contemplated in the context of the invention is the collection of blood samples or apheresis product from a subject at a time period prior to when the expanded cells as described herein might be needed. As such, the source of the cells to be expanded can be collected at any time point necessary, and desired cells, such as T cells, isolated and frozen for later use in immune effector cell therapy for any number of diseases or conditions that would benefit from immune effector cell therapy, such as those described herein. In some embodiments a blood sample or an apheresis is taken from a generally healthy subject. In some embodiments, a blood sample or an apheresis is taken from a generally healthy subject who is at risk of developing a disease, but who has not yet developed a disease, and the cells of interest are isolated and frozen for later use. In some embodiments, the T cells may be expanded, frozen, and used at a later time. In some embodiments, samples are collected from a patient shortly after diagnosis of a particular disease as described herein but prior to any treatments. In some embodiments, the cells are isolated from a blood sample or an apheresis from a subject prior to any number of relevant treatment modalities, including but not limited to treatment with agents such as natalizumab, efalizumab, antiviral agents, chemotherapy, radiation, immunosuppressive agents, such as cyclosporin, azathioprine, methotrexate, mycophenolate, and FK506, antibodies, or other immunoablative agents such as CAMPATH, anti-CD3 antibodies, cytoxan, fludarabine, cyclosporin, FK506, rapamycin, mycophenolic acid, steroids, FR901228, and irradiation.

In some embodiments of the present invention, T cells are obtained from a patient directly following treatment that leaves the subject with functional T cells. In this regard, it has been observed that following certain cancer treatments, in particular treatments with drugs that damage the immune system, shortly after treatment during the period when patients would normally be recovering from the treatment, the quality of T cells obtained may be optimal or improved for their ability to expand ex vivo. Likewise, following ex vivo manipulation using the methods described herein, these cells may be in a preferred state for enhanced engraftment and in vivo expansion. Thus, it is contemplated within the context of the present invention to collect blood cells, including T cells, dendritic cells, or other cells of the hematopoietic lineage, during this recovery phase. Further, in some embodiments, mobilization (for example, mobilization with GM-CSF) and conditioning regimens can be used to create a condition in a subject wherein repopulation, recirculation, regeneration, and/or expansion of particular cell types is favored, especially during a defined window of time following therapy. Illustrative cell types include T cells, B cells, dendritic cells, and other cells of the immune system.

In some embodiments, the immune effector cells expressing a CAR molecule, for example, a CAR molecule described herein, are obtained from a subject that has received a low, immune enhancing dose of an mTOR inhibitor. In some embodiments, the population of immune effector cells, for example, T cells, to be engineered to express a CAR, are harvested after a sufficient time, or after sufficient dosing of the low, immune enhancing, dose of an mTOR inhibitor, such that the level of PD1 negative immune effector cells, for example, T cells, or the ratio of PD1 negative immune effector cells, for example, T cells/PD1 positive immune effector cells, for example, T cells, in the subject or harvested from the subject has been, at least transiently, increased.

In other embodiments, population of immune effector cells, for example, T cells, which have, or will be engineered to express a CAR, can be treated ex vivo by contact with an amount of an mTOR inhibitor that increases the number of PD1 negative immune effector cells, for example, T cells or increases the ratio of PD1 negative immune effector cells, for example, T cells/PD1 positive immune effector cells, for example, T cells.

It is recognized that the methods of the application can utilize culture media conditions comprising 5% or less, for example 2%, human AB serum, and employ known culture media conditions and compositions, for example those described in Smith et al., “Ex vivo expansion of human T cells for adoptive immunotherapy using the novel Xeno-free CTS™ Immune Cell Serum Replacement” Clinical & Translational Immunology (2015) 4, e31; doi:10.1038/cti.2014.31.

In some embodiments, the methods of the application can utilize media conditions comprising at least about 0.1%, 0.5%, 1.0%, 1.5%, 2%, 2.5%, 3%, 3.5%, 4%, 4.5%, 5%, 6%, 7%, 8%, 9% or 10% serum. In some embodiments, the media comprises about 0.5%-5%, about 0.5%-4.5%, about 0.5%-4%, about 0.5%-3.5%, about 0.5%-3%, about 0.5%-2.5%, about 0.5%-2%, about 0.5%-1.5%, about 0.5%-1.0%, about 1.0%-5%, about 1.5%-5%, about 2%-5%, about 2.5%-5%, about 3%-5%, about 3.5%-5%, about 4%-5%, or about 4.5%-5% serum. In some embodiments, the media comprises about 0.5% serum. In some embodiments, the media comprises about 0.5% serum. In some embodiments, the media comprises about 1% serum. In some embodiments, the media comprises about 1.5% serum. In some embodiments, the media comprises about 2% serum. In some embodiments, the media comprises about 2.5% serum. In some embodiments, the media comprises about 3% serum. In some embodiments, the media comprises about 3.5% serum. In some embodiments, the media comprises about 4% serum. In some embodiments, the media comprises about 4.5% serum. In some embodiments, the media comprises about 5% serum. In some embodiments, the serum comprises human serum, e.g., human AB serum. In some embodiments, the serum is human serum that has been allowed to naturally coagulate after collection, e.g., off-the-clot (OTC) serum. In some embodiments, the serum is plasma-derived serum human serum. Plasma-derived serum can be produced by defibrinating pooled human plasma collected in the presence of an anticoagulant, e.g., sodium citrate.

In some embodiments, the methods of the application can utilize culture media conditions comprising serum-free medium. In some embodiments, the serum free medium is OpTmizer™ CTS™ (LifeTech), Immunocult™ XF (Stemcell technologies), CellGro™ (CellGenix), TexMacs™ (Miltenyi), Stemline™ (Sigma), Xvivol5™ (Lonza), PrimeXV® (Irvine Scientific), or StemXVivo® (RandD systems). The serum-free medium can be supplemented with a serum substitute such as ICSR (immune cell serum replacement) from LifeTech. The level of serum substitute (for example, ICSR) can be, for example, up to 5%, for example, about 1%, 2%, 3%, 4%, or 5%. In some embodiments, the serum-free medium can be supplemented with serum, e.g., human serum, e.g., human AB serum. In some embodiments, the serum is human serum that has been allowed to naturally coagulate after collection, e.g., off-the-clot (OTC) serum. In some embodiments, the serum is plasma-derived human serum. Plasma-derived serum can be produced by defibrinating pooled human plasma collected in the presence of an anticoagulant, e.g., sodium citrate.

In some embodiments, a T cell population is diglycerol kinase (DGK)-deficient. DGK-deficient cells include cells that do not express DGK RNA or protein, or have reduced or inhibited DGK activity. DGK-deficient cells can be generated by genetic approaches, for example, administering RNA-interfering agents, for example, siRNA, shRNA, miRNA, to reduce or prevent DGK expression. Alternatively, DGK-deficient cells can be generated by treatment with DGK inhibitors described herein.

In some embodiments, a T cell population is Ikaros-deficient. Ikaros-deficient cells include cells that do not express Ikaros RNA or protein, or have reduced or inhibited Ikaros activity, Ikaros-deficient cells can be generated by genetic approaches, for example, administering RNA-interfering agents, for example, siRNA, shRNA, miRNA, to reduce or prevent Ikaros expression. Alternatively, Ikaros-deficient cells can be generated by treatment with Ikaros inhibitors, for example, lenalidomide.

In embodiments, a T cell population is DGK-deficient and Ikaros-deficient, for example, does not express DGK and Ikaros, or has reduced or inhibited DGK and Ikaros activity. Such DGK and Ikaros-deficient cells can be generated by any of the methods described herein.

In some embodiments, the NK cells are obtained from the subject. In some embodiments, the NK cells are an NK cell line, for example, NK-92 cell line (Conkwest).

### Allogeneic CAR-Expressing Cells

In embodiments described herein, the immune effector cell can be an allogeneic immune effector cell, for example, T cell or NK cell. For example, the cell can be an allogeneic T cell, for example, an allogeneic T cell lacking expression of a functional T cell receptor (TCR) and/or human leukocyte antigen (HLA), for example, HLA class I and/or HLA class II.

A T cell lacking a functional TCR can be, for example, engineered such that it does not express any functional TCR on its surface, engineered such that it does not express one or more subunits that comprise a functional TCR (for example, engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, for example, by expression of mutated or truncated forms of one or more of the subunits of the TCR. The term “substantially impaired TCR” means that this TCR will not elicit an adverse immune reaction in a host.

A T cell described herein can be, for example, engineered such that it does not express a functional HLA on its surface. For example, a T cell described herein, can be engineered such that cell surface expression HLA, for example, HLA class 1 and/or HLA class II, is downregulated. In some embodiments, downregulation of HLA may be accomplished by reducing or eliminating expression of beta-2 microglobulin (B2M).

In some embodiments, the T cell can lack a functional TCR and a functional HLA, for example, HLA class I and/or HLA class II.

Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not express or expresses at low levels an inhibitory molecule, for example by any method described herein. For example, the cell can be a cell that does not express or expresses at low levels an inhibitory molecule, for example, that can decrease the ability of a CAR-expressing cell to mount an immune effector response. Examples of inhibitory molecules include PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF (for example, TGF beta). Inhibition of an inhibitory molecule, for example, by inhibition at the DNA, RNA or protein level, can optimize a CAR-expressing cell performance. In embodiments, an inhibitory nucleic acid, for example, an inhibitory nucleic acid, for example, a dsRNA, for example, an siRNA or shRNA, a clustered regularly interspaced short palindromic repeats (CRISPR), a transcription-activator like effector nuclease (TALEN), or a zinc finger endonuclease (ZFN), for example, as described herein, can be used.

siRNA and shRNA to Inhibit TCR or HLA

In some embodiments, TCR expression and/or HLA expression can be inhibited using siRNA or shRNA that targets a nucleic acid encoding a TCR and/or HLA, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

Expression systems for siRNA and shRNAs, and exemplary shRNAs, are described, for example, in paragraphs 649 and 650 of International Application WO2015/142675, filed Mar. 13, 2015, which is incorporated by reference in its entirety.

### CRISPR to Inhibit TCR or HLA

“CRISPR” or “CRISPR to TCR and/or HLA” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

The CRISPR/Cas system, and uses thereof, are described, for example, in paragraphs 651-658 of International Application WO2015/142675, filed Mar. 13, 2015, which is incorporated by reference in its entirety.

TALEN to Inhibit TCR and/or HLA

“TALEN” or “TALEN to HLA and/or TCR” or “TALEN to inhibit HLA and/or TCR” refers to a transcription activator-like effector nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

TALENs, and uses thereof, are described, for example, in paragraphs 659-665 of International Application WO2015/142675, filed Mar. 13, 2015, which is incorporated by reference in its entirety.

Zinc Finger Nuclease to Inhibit HLA and/or TCR

“ZFN” or “Zinc Finger Nuclease” or “ZFN to HLA and/or TCR” or “ZFN to inhibit HLA and/or TCR” refer to a zinc finger nuclease, an artificial nuclease which can be used to edit the HLA and/or TCR gene, and/or an inhibitory molecule described herein (for example, PD1, PD-L1, PD-L2, CTLA4, TIM3, CEACAM (for example, CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGF beta), in a cell, for example, T cell.

ZFNs, and uses thereof, are described, for example, in paragraphs 666-671 of International Application WO2015/142675, filed Mar. 13, 2015, which is incorporated by reference in its entirety.

### Telomerase Expression

Telomeres play a crucial role in somatic cell persistence, and their length is maintained by telomerase (TERT). Telomere length in CLL cells may be very short (Roth et al., “Significantly shorter telomeres in T-cells of patients with ZAP-70+/CD38 chronic lymphocytic leukaemia” British Journal of Haematology, 143, 383-386, Aug. 28, 2008), and may be even shorter in manufactured CAR-expressing cells, for example, CART19 cells, limiting their potential to expand after adoptive transfer to a patient. Telomerase expression can rescue CAR-expressing cells from replicative exhaustion.

While not wishing to be bound by any particular theory, in some embodiments, a therapeutic T cell has short term persistence in a patient, due to shortened telomeres in the T cell; accordingly, transfection with a telomerase gene can lengthen the telomeres of the T cell and improve persistence of the T cell in the patient. See Carl June, “Adoptive T cell therapy for cancer in the clinic”, Journal of Clinical Investigation, 117:1466-1476 (2007). Thus, in some embodiments, an immune effector cell, for example, a T cell, ectopically expresses a telomerase subunit, for example, the catalytic subunit of telomerase, for example, TERT, for example, hTERT. In some embodiments, this disclosure provides a method of producing a CAR-expressing cell, comprising contacting a cell with a nucleic acid encoding a telomerase subunit, for example, the catalytic subunit of telomerase, for example, TERT, for example, hTERT. The cell may be contacted with the nucleic acid before, simultaneous with, or after being contacted with a construct encoding a CAR.

Telomerase expression may be stable (for example, the nucleic acid may integrate into the cell's genome) or transient (for example, the nucleic acid does not integrate, and expression declines after a period of time, for example, several days). Stable expression may be accomplished by transfecting or transducing the cell with DNA encoding the telomerase subunit and a selectable marker, and selecting for stable integrants. Alternatively, or in combination, stable expression may be accomplished by site-specific recombination, for example, using the Cre/Lox or FLP/FRT system.

Transient expression may involve transfection or transduction with a nucleic acid, for example, DNA or RNA such as mRNA. In some embodiments, transient mRNA transfection avoids the genetic instability sometimes associated with stable transfection with TERT. Transient expression of exogenous telomerase activity is described, for example, in International Application WO2014/130909, which is incorporated by reference herein in its entirety. In embodiments, mRNA-based transfection of a telomerase subunit is performed according to the messenger RNA Therapeutics™ platform commercialized by Moderna Therapeutics. For instance, the method may be a method described in U.S. Pat. Nos. 8,710,200, 8,822,663, 8,680,069, 8,754,062, 8,664,194, or 8680069.

In some embodiments, hTERT has the amino acid sequence of GenBank Protein ID AAC51724.1 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795):

(SEQ ID NO: 284)
MPRAPRCRAVRSLLRSHYREVLPLATFVRRLGPQGWRLVQRGDPAAFRAL
VAQCLVCVPWDARPPPAAPSFRQVSCLKELVARVLQRLCERGAKNVLAFG
FALLDGARGGPPEAFTTSVRSYLPNTVTDALRGSGAWGLLLRRVGDDVLV
HLLARCALFVLVAPSCAYQVCGPPLYQLGAATQARPPPHASGPRRRLGCE
RAWNHSVREAGVPLGLPAPGARRRGGSASRSLPLPKRPRRGAAPEPERTP
VGQGSWAHPGRTRGPSDRGFCVVSPARPAEEATSLEGALSGTRHSHPSVG
RQHHAGPPSTSRPPRPWDTPCPPVYAETKHFLYSSGDKEQLRPSFLLSSL
RPSLTGARRLVETIFLGSRPWMPGTPRRLPRLPQRYWQMRPLFLELLGNH
AQCPYGVLLKTHCPLRAAVTPAAGVCAREKPQGSVAAPEEEDTDPRRLVQ
LLRQHSSPWQVYGFVRACLRRLVPPGLWGSRHNERRFLRNTKKFISLGKH
AKLSLQELTWKMSVRGCAWLRRSPGVGCVPAAEHRLREEILAKFLHWLMS
VYVVELLRSFFYVTETTFQKNRLFFYRKSVWSKLQSIGIRQHLKRVQLRE
LSEAEVRQHREARPALLTSRLRFIPKPDGLRPIVNMDYVVGARTFRREKR
AERLTSRVKALFSVLNYERARRPGLLGASVLGLDDIHRAWRTFVLRVRAQ
DPPPELYFVKVDVTGAYDTIPQDRLTEVIASIIKPONTYCVRRYAVVQKA
AHGHVRKAFKSHVSTLTDLQPYMRQFVAHLQETSPLRDAVVIEQSSSLNE
ASSGLFDVFLRFMCHHAVRIRGKSYVQCQGIPQGSILSTLLCSLCYGDME
NKLFAGIRRDGLLLRLVDDFLLVTPHLTHAKTFLRTLVRGVPEYGCVVNL
RKTVVNFPVEDEALGGTAFVQMPAHGLFPWCGLLLDTRTLEVQSDYSSYA
RTSIRASLTFNRGFKAGRNMRRKLFGVLRLKCHSLFLDLQVNSLQTVCTN
IYKILLLQAYRFHACVLQLPFHQQVWKNPTFFLRVISDTASLCYSILKAK
NAGMSLGAKGAAGPLPSEAVQWLCHQAFLLKLTRHRVTYVPLLGSLRTAQ
TQLSRKLPGTTLTALEAAANPALPSDFKTILD

In some embodiments, the hTERT has a sequence at least 80%, 85%, 90%, 95%, 96%, 97%, 98%, or 99% identical to the sequence of SEQ ID NO: 284. In some embodiments, the hTERT has a sequence of SEQ ID NO: 284. In some embodiments, the hTERT comprises a deletion (for example, of no more than 5, 10, 15, 20, or 30 amino acids) at the N-terminus, the C-terminus, or both. In some embodiments, the hTERT comprises a transgenic amino acid sequence (for example, of no more than 5, 10, 15, 20, or 30 amino acids) at the N-terminus, the C-terminus, or both.

In some embodiments, the hTERT is encoded by the nucleic acid sequence of GenBank Accession No. AF018167 (Meyerson et al., “hEST2, the Putative Human Telomerase Catalytic Subunit Gene, Is Up-Regulated in Tumor Cells and during Immortalization” Cell Volume 90, Issue 4, 22 Aug. 1997, Pages 785-795).

### Activation and Expansion of Immune Effector Cells (for Example, T Cells)

Immune effector cells such as T cells generated or enriched by the methods described herein may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, a population of immune effector cells may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (for example, bryostatin) in conjunction with a calcium ionophore. For costimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besangon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In some embodiments, the agent providing the costimulatory signal is bound to a cell surface and the agent providing the primary activation signal is in solution or coupled to a surface. In some embodiments, both agents can be in solution. In some embodiments, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In some embodiments, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In some embodiments, a 1:1 ratio of each antibody bound to the beads for CD4+ T cell expansion and T cell growth is used. In some embodiments of the present invention, a ratio of anti CD3:CD28 antibodies bound to the beads is used such that an increase in T cell expansion is observed as compared to the expansion observed using a ratio of 1:1. In some embodiments an increase of from about 1 to about 3 fold is observed as compared to the expansion observed using a ratio of 1:1. In some embodiments, the ratio of CD3:CD28 antibody bound to the beads ranges from 100:1 to 1:100 and all integer values there between. In some embodiments, more anti-CD28 antibody is bound to the particles than anti-CD3 antibody, i.e., the ratio of CD3:CD28 is less than one. In some embodiments, the ratio of anti CD28 antibody to anti CD3 antibody bound to the beads is greater than 2:1. In some embodiments, a 1:100 CD3:CD28 ratio of antibody bound to beads is used. In some embodiments, a 1:75 CD3:CD28 ratio of antibody bound to beads is used. In some embodiments, a 1:50 CD3:CD28 ratio of antibody bound to beads is used. In some embodiments, a 1:30 CD3:CD28 ratio of antibody bound to beads is used. In some embodiments, a 1:10 CD3:CD28 ratio of antibody bound to beads is used. In some embodiments, a 1:3 CD3:CD28 ratio of antibody bound to the beads is used. In some embodiments, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In some embodiments the ratio of cells to particles ranges from 1:100 to 100:1 and any integer values in-between and in some embodiments the ratio comprises 1:9 to 9:1 and any integer values in between, can also be used to stimulate T cells. The ratio of anti-CD3- and anti-CD28-coupled particles to T cells that result in T cell stimulation can vary as noted above, however certain suitable values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one suitable ratio being at least 1:1 particles per T cell. In some embodiments, a ratio of particles to cells of 1:1 or less is used. In some embodiments, a suitable particle:cell ratio is 1:5. In some embodiments, the ratio of particles to cells can be varied depending on the day of stimulation. For example, in some embodiments, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In some embodiments, the ratio of particles to cells is 1:1 on the first day of stimulation and adjusted to 1:5 on the third and fifth days of stimulation. In some embodiments, particles are added on a daily or every other day basis to a final ratio of 1:1 on the first day, and 1:5 on the third and fifth days of stimulation. In some embodiments, the ratio of particles to cells is 2:1 on the first day of stimulation and adjusted to 1:10 on the third and fifth days of stimulation. In some embodiments, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In some embodiments, the most typical ratios for use are in the neighborhood of 1:1, 2:1 and 3:1 on the first day.

In some embodiments, the cells, such as T cells, are combined with agent-coated beads, the beads and the cells are subsequently separated, and then the cells are cultured. In some embodiments, prior to culture, the agent-coated beads and cells are not separated but are cultured together. In some embodiments, the beads and cells are first concentrated by application of a force, such as a magnetic force, resulting in increased ligation of cell surface markers, thereby inducing cell stimulation.

By way of example, cell surface proteins may be ligated by allowing paramagnetic beads to which anti-CD3 and anti-CD28 are attached (3×28 beads) to contact the T cells. In some embodiments the cells (for example, 10′ to 10′ T cells) and beads (for example, Dynabeads® M-450 CD3/CD28 T paramagnetic beads at a ratio of 1:1) are combined in a buffer, for example PBS (without divalent cations such as, calcium and magnesium). Again, those of ordinary skill in the art can readily appreciate any cell concentration may be used. For example, the target cell may be very rare in the sample and comprise only 0.01% of the sample or the entire sample (i.e., 100%) may comprise the target cell of interest. Accordingly, any cell number is within the context of the present invention. In some embodiments, it may be desirable to significantly decrease the volume in which particles and cells are mixed together (i.e., increase the concentration of cells), to ensure maximum contact of cells and particles. For example, in some embodiments, a concentration of about 10 billion cells/ml, 9 billion/ml, 8 billion/ml, 7 billion/ml, 6 billion/ml, 5 billion/ml, or 2 billion cells/ml is used. In some embodiments, greater than 100 million cells/ml is used. In some embodiments, a concentration of cells of 10, 15, 20, 25, 30, 35, 40, 45, or 50 million cells/ml is used. In some embodiments, a concentration of cells from 75, 80, 85, 90, 95, or 100 million cells/ml is used. In some embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells. Such populations of cells may have therapeutic value and would be desirable to obtain in some embodiments. For example, using high concentration of cells allows more efficient selection of CD8+ T cells that normally have weaker CD28 expression.

In some embodiments, cells transduced with a nucleic acid encoding a CAR, for example, a CAR described herein, for example, a CD19 CAR described herein, are expanded, for example, by a method described herein. In some embodiments, the cells are expanded in culture for a period of several hours (for example, about 2, 3, 4, 5, 6, 7, 8, 9, 10, 15, 18, 21 hours) to about 14 days (for example, 1, 2, 3, 4, 5, 6, 7, 8, 9, 10, 11, 12, 13 or 14 days). In some embodiments, the cells are expanded for a period of 4 to 9 days. In some embodiments, the cells are expanded for a period of 8 days or less, for example, 7, 6 or 5 days. In some embodiments, the cells are expanded in culture for 5 days, and the resulting cells are more potent than the same cells expanded in culture for 9 days under the same culture conditions. Potency can be defined, for example, by various T cell functions, for example proliferation, target cell killing, cytokine production, activation, migration, surface CAR expression, CAR quantitative PCR, or combinations thereof. In some embodiments, the cells, for example, a CD19 CAR cell described herein, expanded for 5 days show at least a one, two, three or four-fold increase in cells doublings upon antigen stimulation as compared to the same cells expanded in culture for 9 days under the same culture conditions. In some embodiments, the cells, for example, the cells expressing a CD19 CAR described herein, are expanded in culture for 5 days, and the resulting cells exhibit higher proinflammatory cytokine production, for example, IFN-γ and/or GM-CSF levels, as compared to the same cells expanded in culture for 9 days under the same culture conditions. In some embodiments, the cells, for example, a CD19 CAR cell described herein, expanded for 5 days show at least a one, two, three, four, five, ten-fold or more increase in pg/ml of proinflammatory cytokine production, for example, IFN-γ and/or GM-CSF levels, as compared to the same cells expanded in culture for 9 days under the same culture conditions.

Several cycles of stimulation may also be desired such that culture time of T cells can be 60 days or more. Conditions appropriate for T cell culture include an appropriate media (for example, Minimal Essential Media, α-MEM, RPMI Media 1640, AIM-V, DMEM, F-12, or X-vivo 15 (Lonza), X-Vivo 20, OpTmizer, and IMDM) that may contain factors necessary for proliferation and viability, including serum (for example, fetal bovine or human serum), interleukin-2 (IL-2), insulin, IFNγ, IL-4, IL-7, GM-CSF, IL-10, IL-12, IL-15, TGFβ, and TNFα or any other additives for the growth of cells known to the skilled artisan. Other additives for the growth of cells include, but are not limited to, surfactant, plasmanate, and reducing agents such as N-acetyl-cysteine and 2-mercaptoethanol. Media can include, but is not limited to RPMI 1640, AIM-V, DMEM, MEM, α-MEM, F-12, X-Vivo 15, X-Vivo 20, OpTmizer, and IMDM with added amino acids, sodium pyruvate, and vitamins, either serum-free or supplemented with an appropriate amount of serum (or plasma) or a defined set of hormones, and/or an amount of cytokine(s) sufficient for the growth and expansion of T cells. Antibiotics, for example, penicillin and streptomycin, are included only in experimental cultures, not in cultures of cells that are to be infused into a subject. The target cells are maintained under conditions necessary to support growth, for example, an appropriate temperature (for example, 37° C.) and atmosphere (for example, air plus 5% CO₂).

In some embodiments, the cells are expanded in an appropriate media (for example, media described herein) that includes one or more interleukin that result in at least a 200-fold (for example, 200-fold, 250-fold, 300-fold, 350-fold) increase in cells over a 14-day expansion period, for example, as measured by a method described herein such as flow cytometry. In some embodiments, the cells are expanded in the presence IL-15 and/or IL-7 (for example, IL-15 and IL-7).

In embodiments, methods described herein, for example, CAR-expressing cell manufacturing methods, comprise removing T regulatory cells, for example, CD25+ T cells or CD25^{high} T cells, from a cell population, for example, using an anti-CD25 antibody, or fragment thereof, or a CD25-binding ligand, IL-2. Methods of removing T regulatory cells, for example, CD25+ T cells or CD25^{high} T cells, from a cell population are described herein. In embodiments, the methods, for example, manufacturing methods, further comprise contacting a cell population (for example, a cell population in which T regulatory cells, such as CD25+ T cells or CD25^{high} T cells, have been depleted; or a cell population that has previously contacted an anti-CD25 antibody, fragment thereof, or CD25-binding ligand) with IL-15 and/or IL-7. For example, the cell population (for example, that has previously contacted an anti-CD25 antibody, fragment thereof, or CD25-binding ligand) is expanded in the presence of IL-15 and/or IL-7.

In some embodiments a CAR-expressing cell described herein is contacted with a composition comprising a interleukin-15 (IL-15) polypeptide, a interleukin-15 receptor alpha (IL-15Ra) polypeptide, or a combination of both a IL-15 polypeptide and a IL-15Ra polypeptide for example, hetIL-15, during the manufacturing of the CAR-expressing cell, for example, ex vivo. In embodiments, a CAR-expressing cell described herein is contacted with a composition comprising an IL-15 polypeptide during the manufacturing of the CAR-expressing cell, for example, ex vivo. In embodiments, a CAR-expressing cell described herein is contacted with a composition comprising a combination of both an IL-15 polypeptide and a IL-15 Ra polypeptide during the manufacturing of the CAR-expressing cell, for example, ex vivo. In embodiments, a CAR-expressing cell described herein is contacted with a composition comprising hetIL-15 during the manufacturing of the CAR-expressing cell, for example, ex vivo.

In some embodiments the CAR-expressing cell described herein is contacted with a composition comprising hetIL-15 during ex vivo expansion. In some embodiments, the CAR-expressing cell described herein is contacted with a composition comprising an IL-15 polypeptide during ex vivo expansion. In some embodiments, the CAR-expressing cell described herein is contacted with a composition comprising both an IL-15 polypeptide and an IL-15Ra polypeptide during ex vivo expansion. In some embodiments the contacting results in the survival and proliferation of a lymphocyte subpopulation, for example, CD8+ T cells.

T cells that have been exposed to varied stimulation times may exhibit different characteristics. For example, typical blood or apheresed peripheral blood mononuclear cell products have a helper T cell population (TH, CD4+) that is greater than the cytotoxic or suppressor T cell population (TC, CD8+). Ex vivo expansion of T cells by stimulating CD3 and CD28 receptors produces a population of T cells that prior to about days 8-9 consists predominately of TH cells, while after about days 8-9, the population of T cells comprises an increasingly greater population of TC cells. Accordingly, depending on the purpose of treatment, infusing a subject with a T cell population comprising predominately of TH cells may be advantageous. Similarly, if an antigen-specific subset of TC cells has been isolated it may be beneficial to expand this subset to a greater degree.

Further, in addition to CD4 and CD8 markers, other phenotypic markers vary significantly, but in large part, reproducibly during the course of the cell expansion process. Thus, such reproducibility enables the ability to tailor an activated T cell product for specific purposes.

Once a CAR described herein is constructed, various assays can be used to evaluate the activity of the molecule, such as but not limited to, the ability to expand T cells following antigen stimulation, sustain T cell expansion in the absence of re-stimulation, and anti-cancer activities in appropriate in vitro and animal models. Assays to evaluate the effects of a CAR of the present invention are described in further detail below

Western blot analysis of CAR expression in primary T cells can be used to detect the presence of monomers and dimers, for example, as described in paragraph 695 of International Application WO2015/142675, filed Mar. 13, 2015, which is herein incorporated by reference in its entirety.

In vitro expansion of CAR⁺ T cells following antigen stimulation can be measured by flow cytometry. For example, a mixture of CD4⁺ and CD8⁺ T cells are stimulated with αCD3/αCD28 aAPCs followed by transduction with lentiviral vectors expressing GFP under the control of the promoters to be analyzed. Exemplary promoters include the CMV IE gene, EF-1α, ubiquitin C, or phosphoglycerokinase (PGK) promoters. GFP fluorescence is evaluated on day 6 of culture in the CD4⁺ and/or CD8⁺ T cell subsets by flow cytometry. See, for example, Milone et al., Molecular Therapy 17(8): 1453-1464 (2009). Alternatively, a mixture of CD4+ and CD8+ T cells are stimulated with αCD3/αCD28 coated magnetic beads on day 0, and transduced with CAR on day 1 using a bicistronic lentiviral vector expressing CAR along with eGFP using a 2A ribosomal skipping sequence. Cultures are re-stimulated with either a cancer associated antigen as described herein⁺ K562 cells (K562-expressing a cancer associated antigen as described herein), wild-type K562 cells (K562 wild type) or K562 cells expressing hCD32 and 4-1BBL in the presence of antiCD3 and anti-CD28 antibody (K562-BBL-3/28). Exogenous IL-2 is added to the cultures every other day at 100 IU/ml. GFP⁺ T cells are enumerated by flow cytometry using bead-based counting. See, for example, Milone et al., Molecular Therapy 17(8): 1453-1464 (2009).

Sustained CAR⁺ T cell expansion in the absence of re-stimulation can also be measured. See, for example, Milone et al., Molecular Therapy 17(8): 1453-1464 (2009). Briefly, mean T cell volume (fl) is measured on day 8 of culture using a Coulter Multisizer III particle counter or a higher version, a Nexcelom Cellometer Vision, Millipore Scepter or other cell counters, following stimulation with αCD3/αCD28 coated magnetic beads on day 0, and transduction with the indicated CAR on day 1.

Animal models can also be used to measure a CAR-expressing cell activity, for example, as described in paragraph 698 of International Application WO2015/142675, filed Mar. 13, 2015, which is herein incorporated by reference in its entirety.

Dose dependent CAR treatment response can be evaluated, for example, as described in paragraph 699 of International Application WO2015/142675, filed Mar. 13, 2015, which is herein incorporated by reference in its entirety.

Assessment of cell proliferation and cytokine production has been previously described, as described in paragraph 700 of International Application WO2015/142675, filed Mar. 13, 2015, which is herein incorporated by reference in its entirety.

Cytotoxicity can be assessed by a standard 51Cr-release assay, for example, as described in paragraph 701 of International Application WO2015/142675, filed Mar. 13, 2015, which is herein incorporated by reference in its entirety. Alternative non-radioactive methods can be utilized as well.

Cytotoxicity can also be assessed by measuring changes in adherent cell's electrical impedance, for example, using an xCELLigence real time cell analyzer (RTCA). In some embodiments, cytotoxicity is measured at multiple time points.

Imaging technologies can be used to evaluate specific trafficking and proliferation of CARs in tumor-bearing animal models, for example, as described in paragraph 702 of International Application WO2015/142675, filed Mar. 13, 2015, which is herein incorporated by reference in its entirety.

Other assays, including those described in the Example section herein as well as those that are known in the art can also be used to evaluate the CARs described herein.

Alternatively, or in combination to the methods disclosed herein, methods and compositions for one or more of: detection and/or quantification of CAR-expressing cells (for example, in vitro or in vivo (for example, clinical monitoring)); immune cell expansion and/or activation; and/or CAR-specific selection, that involve the use of a CAR ligand, are disclosed. In some embodiments, the CAR ligand is an antibody that binds to the CAR molecule, for example, binds to the extracellular antigen binding domain of CAR (for example, an antibody that binds to the antigen binding domain, for example, an anti-idiotypic antibody; or an antibody that binds to a constant region of the extracellular binding domain). In other embodiments, the CAR ligand is a CAR antigen molecule (for example, a CAR antigen molecule as described herein).

In some embodiments, a method for detecting and/or quantifying CAR-expressing cells is disclosed. For example, the CAR ligand can be used to detect and/or quantify CAR-expressing cells in vitro or in vivo (for example, clinical monitoring of CAR-expressing cells in a patient, or dosing a patient). The method includes:
providing the CAR ligand (optionally, a labelled CAR ligand, for example, a CAR ligand that includes a tag, a bead, a radioactive or fluorescent label);
acquiring the CAR-expressing cell (for example, acquiring a sample containing CAR-expressing cells, such as a manufacturing sample or a clinical sample);
contacting the CAR-expressing cell with the CAR ligand under conditions where binding occurs, thereby detecting the level (for example, amount) of the CAR-expressing cells present. Binding of the CAR-expressing cell with the CAR ligand can be detected using standard techniques such as FACS, ELISA and the like.

In some embodiments, a method of expanding and/or activating cells (for example, immune effector cells) is disclosed. The method includes:
providing a CAR-expressing cell (for example, a first CAR-expressing cell or a transiently expressing CAR cell);
contacting said CAR-expressing cell with a CAR ligand, for example, a CAR ligand as described herein), under conditions where immune cell expansion and/or proliferation occurs, thereby producing the activated and/or expanded cell population.

In certain embodiments, the CAR ligand is present on a substrate (for example, is immobilized or attached to a substrate, for example, a non-naturally occurring substrate). In some embodiments, the substrate is a non-cellular substrate. The non-cellular substrate can be a solid support chosen from, for example, a plate (for example, a microtiter plate), a membrane (for example, a nitrocellulose membrane), a matrix, a chip or a bead. In embodiments, the CAR ligand is present in the substrate (for example, on the substrate surface). The CAR ligand can be immobilized, attached, or associated covalently or non-covalently (for example, cross-linked) to the substrate. In some embodiments, the CAR ligand is attached (for example, covalently attached) to a bead. In the aforesaid embodiments, the immune cell population can be expanded in vitro or ex vivo. The method can further include culturing the population of immune cells in the presence of the ligand of the CAR molecule, for example, using any of the methods described herein.

In other embodiments, the method of expanding and/or activating the cells further comprises addition of a second stimulatory molecule, for example, CD28. For example, the CAR ligand and the second stimulatory molecule can be immobilized to a substrate, for example, one or more beads, thereby providing increased cell expansion and/or activation.

In some embodiments, a method for selecting or enriching for a CAR expressing cell is provided. The method includes contacting the CAR expressing cell with a CAR ligand as described herein; and selecting the cell on the basis of binding of the CAR ligand.

In yet other embodiments, a method for depleting, reducing and/or killing a CAR expressing cell is provided. The method includes contacting the CAR expressing cell with a CAR ligand as described herein; and targeting the cell on the basis of binding of the CAR ligand, thereby reducing the number, and/or killing, the CAR-expressing cell. In some embodiments, the CAR ligand is coupled to a toxic agent (for example, a toxin or a cell ablative drug). In some embodiments, the anti-idiotypic antibody can cause effector cell activity, for example, ADCC or ADC activities.

Exemplary anti-CAR antibodies that can be used in the methods disclosed herein are described, for example, in WO 2014/190273 and by Jena et al., “Chimeric Antigen Receptor (CAR)-Specific Monoclonal Antibody to Detect CD19-Specific T cells in Clinical Trials”, PLOS March 2013 8:3 e57838, the contents of which are incorporated by reference.

In some embodiments, the compositions and methods herein are optimized for a specific subset of T cells, for example, as described in US Serial No. PCT/US2015/043219 filed Jul. 31, 2015, the contents of which are incorporated herein by reference in their entirety. In some embodiments, the optimized subsets of T cells display an enhanced persistence compared to a control T cell, for example, a T cell of a different type (for example, CD8+ or CD4+) expressing the same construct.

In some embodiments, a CD4+ T cell comprises a CAR described herein, which CAR comprises an intracellular signaling domain suitable for (for example, optimized for, for example, leading to enhanced persistence in) a CD4+ T cell, for example, an ICOS domain. In some embodiments, a CD8+ T cell comprises a CAR described herein, which CAR comprises an intracellular signaling domain suitable for (for example, optimized for, for example, leading to enhanced persistence of) a CD8+ T cell, for example, a 4-1BB domain, a CD28 domain, or another costimulatory domain other than an ICOS domain. In some embodiments, the CAR described herein comprises an antigen binding domain described herein, for example, a CAR comprising an antigen binding domain.

In some embodiments, described herein is a method of treating a subject, for example, a subject having cancer. The method includes administering to said subject, an effective amount of:
1) a CD4+ T cell comprising a CAR (the CARCD4+) comprising:
an antigen binding domain, for example, an antigen binding domain described herein;
a transmembrane domain; and
an intracellular signaling domain, for example, a first costimulatory domain, for example, an ICOS domain; and
2) a CD8+ T cell comprising a CAR (the CARCD8+) comprising:
an antigen binding domain, for example, an antigen binding domain described herein;
a transmembrane domain; and
an intracellular signaling domain, for example, a second costimulatory domain, for example, a 4-1BB domain, a CD28 domain, or another costimulatory domain other than an ICOS domain;
wherein the CARCD4+ and the CARCD8+ differ from one another.

### Optionally, the Method Further Includes Administering:

3) a second CD8+ T cell comprising a CAR (the second CARCD8+) comprising:
an antigen binding domain, for example, an antigen binding domain described herein;
a transmembrane domain; and
an intracellular signaling domain, wherein the second CARCD8+ comprises an intracellular signaling domain, for example, a costimulatory signaling domain, not present on the CARCD8+, and, optionally, does not comprise an ICOS signaling domain.

### Biopolymer Delivery Methods

In some embodiments, one or more CAR-expressing cells as disclosed herein can be administered or delivered to the subject via a biopolymer scaffold, for example, a biopolymer implant. Biopolymer scaffolds can support or enhance the delivery, expansion, and/or dispersion of the CAR-expressing cells described herein. A biopolymer scaffold comprises a biocompatible (for example, does not substantially induce an inflammatory or immune response) and/or a biodegradable polymer that can be naturally occurring or synthetic. Exemplary biopolymers are described, for example, in paragraphs 1004-1006 of International Application WO2015/142675, filed Mar. 13, 2015, which is herein incorporated by reference in its entirety.

### Pharmaceutical Compositions and Treatments

In some embodiments, the disclosure provides a method of treating a patient, comprising administering CAR-expressing cells produced as described herein, optionally in combination with one or more other therapies. In some embodiments, the disclosure provides a method of treating a patient, comprising administering a reaction mixture comprising CAR-expressing cells as described herein, optionally in combination with one or more other therapies. In some embodiments, the disclosure provides a method of shipping or receiving a reaction mixture comprising CAR-expressing cells as described herein. In some embodiments, the disclosure provides a method of treating a patient, comprising receiving a CAR-expressing cell that was produced as described herein, and further comprising administering the CAR-expressing cell to the patient, optionally in combination with one or more other therapies. In some embodiments, the disclosure provides a method of treating a patient, comprising producing a CAR-expressing cell as described herein, and further comprising administering the CAR-expressing cell to the patient, optionally in combination with one or more other therapies. The other therapy may be, for example, a cancer therapy such as chemotherapy.

In some embodiments, cells expressing a CAR described herein are administered to a subject in combination with a molecule that decreases the Treg cell population. Methods that decrease the number of (for example, deplete) Treg cells are known in the art and include, for example, CD25 depletion, cyclophosphamide administration, modulating GITR function. Without wishing to be bound by theory, it is believed that reducing the number of Treg cells in a subject prior to apheresis or prior to administration of a CAR-expressing cell described herein reduces the number of unwanted immune cells (for example, Tregs) in the tumor microenvironment and reduces the subject's risk of relapse.

In some embodiments, a therapy described herein, for example, a CAR-expressing cell, is administered to a subject in combination with a molecule targeting GITR and/or modulating GITR functions, such as a GITR agonist and/or a GITR antibody that depletes regulatory T cells (Tregs). In embodiments, cells expressing a CAR described herein are administered to a subject in combination with cyclophosphamide. In some embodiments, the GITR binding molecules and/or molecules modulating GITR functions (for example, GITR agonist and/or Treg depleting GITR antibodies) are administered prior to the CAR-expressing cell. For example, in some embodiments, a GITR agonist can be administered prior to apheresis of the cells. In embodiments, cyclophosphamide is administered to the subject prior to administration (for example, infusion or re-infusion) of the CAR-expressing cell or prior to apheresis of the cells. In embodiments, cyclophosphamide and an anti-GITR antibody are administered to the subject prior to administration (for example, infusion or re-infusion) of the CAR-expressing cell or prior to apheresis of the cells. In some embodiments, the subject has cancer (for example, a solid cancer or a hematological cancer such as ALL or CLL). In some embodiments, the subject has CLL. In embodiments, the subject has ALL. In embodiments, the subject has a solid cancer, for example, a solid cancer described herein. Exemplary GITR agonists include, for example, GITR fusion proteins and anti-GITR antibodies (for example, bivalent anti-GITR antibodies) such as, for example, a GITR fusion protein described in U.S. Pat. No. 6,111,090, European Patent No.: 090505B1, U.S. Pat. No. 8,586,023, PCT Publication Nos.: WO 2010/003118 and 2011/090754, or an anti-GITR antibody described, for example, in U.S. Pat. No. 7,025,962, European Patent No.: 1947183B1, U.S. Pat. Nos. 7,812,135, 8,388,967, 8,591,886, European Patent No.: EP 1866339, PCT Publication No.: WO 2011/028683, PCT Publication No.: WO 2013/039954, PCT Publication No.: WO2005/007190, PCT Publication No.: WO 2007/133822, PCT Publication No.: WO2005/055808, PCT Publication No.: WO 99/40196, PCT Publication No.: WO 2001/03720, PCT Publication No.: WO99/20758, PCT Publication No.: WO2006/083289, PCT Publication No.: WO 2005/115451, U.S. Pat. No. 7,618,632, and PCT Publication No.: WO 2011/051726.

In some embodiments, a CAR expressing cell described herein is administered to a subject in combination with a GITR agonist, for example, a GITR agonist described herein. In some embodiments, the GITR agonist is administered prior to the CAR-expressing cell. For example, in some embodiments, the GITR agonist can be administered prior to apheresis of the cells. In some embodiments, the subject has CLL.

The methods described herein can further include formulating a CAR-expressing cell in a pharmaceutical composition. Pharmaceutical compositions may comprise a CAR-expressing cell, for example, a plurality of CAR-expressing cells, as described herein, in combination with one or more pharmaceutically or physiologically acceptable carriers, diluents or excipients. Such compositions may comprise buffers such as neutral buffered saline, phosphate buffered saline and the like; carbohydrates such as glucose, mannose, sucrose or dextrans, mannitol; proteins; polypeptides or amino acids such as glycine; antioxidants; chelating agents such as EDTA or glutathione; adjuvants (for example, aluminum hydroxide); and preservatives. Compositions can be formulated, for example, for intravenous administration.

In some embodiments, the pharmaceutical composition is substantially free of, for example, there are no detectable levels of a contaminant, for example, selected from the group consisting of endotoxin, mycoplasma, replication competent lentivirus (RCL), p24, VSV-G nucleic acid, HIV gag, residual anti-CD3/anti-CD28 coated beads, mouse antibodies, pooled human serum, bovine serum albumin, bovine serum, culture media components, vector packaging cell or plasmid components, a bacterium and a fungus. In some embodiments, the bacterium is at least one selected from the group consisting of Alcaligenes faecalis, Candida albicans, Escherichia coli, Haemophilus influenza, Neisseria meningitides, Pseudomonas aeruginosa, Staphylococcus aureus, Streptococcus pneumonia, and Streptococcus pyogenes group A.

When “an immunologically effective amount,” “an anti-cancer effective amount,” “a cancer-inhibiting effective amount,” or “therapeutic amount” is indicated, the precise amount of the compositions to be administered can be determined by a physician with consideration of individual differences in age, weight, tumor size, extent of infection or metastasis, and condition of the patient (subject). It can generally be stated that a pharmaceutical composition comprising the immune effector cells (for example, T cells, NK cells) described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, for example, Rosenberg et al., New Eng. J. of Med. 319:1676, 1988).

### Exemplary BCMA/CD19 CART Pharmaceutical Compositions

In some embodiments a BCMA/CD19 dual CART cell composition is produced by co-transduction with two unique vectors. Accordingly, in some embodiments, the cell composition comprises a heterogeneous population of cells. In some embodiments, the heterogeneous cell composition includes untransduced T cells, mono BCMA-specific CART cells, mono CD19-specific CART cells, and dual CART cells expressing both BCMA-specific CAR molecules and CD19-specific CAR molecules. These distinct cell populations may exhibit different activities in the context of treating a disease in a subject.

Without wishing to be bound by theory, in the context of treating multiple myeloma, for example, activation of BCMA-specific CART cells can be a main factor in the anti-tumor response in patients, while CD19-specific activity can play a role in eliminating less-prevalent CD19-positive tumor cells.

In some embodiments, the cell composition can be evaluated to assess the relative percentages of the four distinct cell populations, e.g., to select a cell composition that contains a greater percentage of BCMA-specific CAR cells (e.g., mono BCMA-specific CAR cells and BCMA/CD19 dual CAR cells) than mono CD19-specific CAR cells.

### In Some Embodiments, the Cell Composition Comprises:

(a) a first population of cells comprising an anti-BCMA CAR but not an anti-CD19 CAR;
(b) a second population of cells comprising an anti-CD19 CAR but not an anti-BCMA CAR; and
(c) a third population of cells comprising both an anti-BCMA CAR and an anti-CD19 CAR.
   In some embodiments:
   (i) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined;
   (ii) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined; and/or
   (iii) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the population.

In some embodiments, the cell composition further comprises a fourth population of cells that do not comprise a CAR. In some embodiments, the cell composition comprises a population of mono CD19-specific CAR cells that are less than or equal to 110% (e.g., less than or equal to about 105%, 100%, 90%, 80%, 70%, 60%, 50%, 40%, 30%, 20%, 10%, 5%, 1%, or less) of the number of BCMA-specific CAR cells (e.g., the total number of mono BCMA-specific CAR cells and BCMA/CD19 dual CAR cells). In some embodiments, the cell composition comprises a population of mono CD19 CAR+ cells that comprise about 45% to about 50% (e.g., about 47%) of the number of BCMA-specific CART cells. In some embodiments, the cell composition comprises a population of mono CD19 CAR+ cells that comprise about 60% to about 65% (e.g., about 63%) of the number of BCMA-specific CART cells. In some embodiments, the cell composition comprises a population of mono CD19 CAR+ cells that comprise about 50% to about 55% (e.g., about 53%) of the number of BCMA-specific CART cells. In some embodiments, the cell composition comprises a population of mono CD19 CAR+ cells that comprise about 82% of the number of BCMA-specific CART cells.

In some embodiments, the cell composition can be evaluated to assess the percentage of CAR-positive viable cells to allow for sufficient dosing. Accordingly, in some embodiments, the cell composition comprises a population of BCMA-specific CAR cells (e.g., the total number of mono BCMA-specific CAR cells and BCMA/CD19 dual CAR cells) that are greater than or equal to about 5% (e.g., greater than or equal to about 10%, 20%, 30%, 40%, 50%, 60%, 70%, 80%, 90%, 95%, 99%, or 100%) of the total number of viable cells in the cell composition.

In some embodiments, the BCMA-specific CAR cells are BCMA-specific CART cells. In some embodiments, the mono BCMA-specific CAR cells are mono BCMA-specific CART cells. In some embodiments, the CD19-specific CAR cells are CD19-specific CART cells. In some embodiments, the mono CD19-specific CAR cells are mono CD19-specific CART cells. In some embodiments, the BCMA/CD19 dual CAR cells are BCMA/CD19 dual CART cells.

### Dosing

In some embodiments, a dose of CAR cells (for example, CD19 CAR cells) comprises about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises at least about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises up to about 1×10⁶, 1.1×10⁶, 2×10⁶, 3.6×10⁶, 5×10⁶, 1×10⁷, 1.8×10⁷, 2×10⁷, 5×10⁷, 1×10⁸, 2×10⁸, or 5×10⁸ cells/kg. In some embodiments, a dose of CAR cells (for example, CD19 CAR cells) comprises about 1.1×10⁶—1.8×10⁷ cells/kg. In some embodiments, a dose of CAR cells (for example, CD19 CAR cells) comprises about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises at least about 1×10⁷, 2×10⁷, 5×10⁷, 1×10⁸, 2×10⁸, 5×10⁸, 1×10⁹, 2×10⁹, or 5×10⁹ cells. In some embodiments, a dose of CAR cells (for example, CD19 CAR cells) comprises up to about 1×10⁷, 2×10⁷, 5×10⁷, 1×10⁸, 2×10⁸, 5×10⁸, 1×10⁹, 2×10⁹, or 5×10⁹ cells.

In some embodiments, it may be desired to administer activated immune effector cells (for example, T cells, NK cells) to a subject and then subsequently redraw blood (or have an apheresis performed), activate immune effector cells (for example, T cells, NK cells) therefrom, and reinfuse the patient with these activated and expanded immune effector cells (for example, T cells, NK cells). This process can be carried out multiple times every few weeks. In some embodiments, immune effector cells (for example, T cells, NK cells) can be activated from blood draws of from 10 cc to 400 cc. In some embodiments, immune effector cells (for example, T cells, NK cells) are activated from blood draws of 20 cc, 30 cc, 40 cc, 50 cc, 60 cc, 70 cc, 80 cc, 90 cc, or 100 cc.

The administration of the subject compositions may be carried out in any convenient manner. The compositions described herein may be administered to a patient trans arterially, subcutaneously, intradermally, intratumorally, intranodally, intramedullary, intramuscularly, by intravenous (i.v.) injection, or intraperitoneally, for example, by intradermal or subcutaneous injection. The compositions of immune effector cells (for example, T cells, NK cells) may be injected directly into a tumor, lymph node, or site of infection.

### T Cell Depletion

In some embodiments, the methods disclosed herein further include administering a T cell depleting agent after treatment with the cell (for example, an immune effector cell as described herein), thereby reducing (for example, depleting) the CAR-expressing cells (for example, the CD19CAR-expressing cells). Such T cell depleting agents can be used to effectively deplete CAR-expressing cells (for example, CD19CAR-expressing cells) to mitigate toxicity. In some embodiments, the CAR-expressing cells were manufactured according to a method herein, for example, assayed (for example, before or after transfection or transduction) according to a method herein.

In some embodiments, the T cell depleting agent is administered one, two, three, four, or five weeks after administration of the cell, for example, the population of immune effector cells, described herein.

In some embodiments, the T cell depleting agent is an agent that depletes CAR-expressing cells, for example, by inducing antibody dependent cell-mediated cytotoxicity (ADCC) and/or complement-induced cell death. For example, CAR-expressing cells described herein may also express an antigen (for example, a target antigen) that is recognized by molecules capable of inducing cell death, for example, ADCC or complement-induced cell death. For example, CAR expressing cells described herein may also express a target protein (for example, a receptor) capable of being targeted by an antibody or antibody fragment. Examples of such target proteins include, but are not limited to, EpCAM, VEGFR, integrins (for example, integrins αvβ3, α4, αI3/4β3, α4β7, α5β1, αvβ3, αv), members of the TNF receptor superfamily (for example, TRAIL-R1, TRAIL-R2), PDGF Receptor, interferon receptor, folate receptor, GPNMB, ICAM-1, HLA-DR, CEA, CA-125, MUC1, TAG-72, IL-6 receptor, 5 T4, GD2, GD3, CD2, CD3, CD4, CD5, CD11, CD11a/LFA-1, CD15, CD18/ITGB2, CD19, CD20, CD22, CD23/lgE Receptor, CD25, CD28, CD30, CD33, CD38, CD40, CD41, CD44, CD51, CD52, CD62L, CD74, CD80, CD125, CD147/basigin, CD152/CTLA-4, CD154/CD40L, CD195/CCR5, CD319/SLAMF7, and EGFR, and truncated versions thereof (for example, versions preserving one or more extracellular epitopes but lacking one or more regions within the cytoplasmic domain).

In some embodiments, the CAR expressing cell co-expresses the CAR and the target protein, for example, naturally expresses the target protein or is engineered to express the target protein. For example, the cell, for example, the population of immune effector cells, can include a nucleic acid (for example, vector) comprising the CAR nucleic acid (for example, a CAR nucleic acid as described herein) and a nucleic acid encoding the target protein.

In some embodiments, the T cell depleting agent is a CD52 inhibitor, for example, an anti-CD52 antibody molecule, for example, alemtuzumab.

In other embodiments, the cell, for example, the population of immune effector cells, expresses a CAR molecule as described herein (for example, CD19CAR) and the target protein recognized by the T cell depleting agent. In some embodiments, the target protein is CD20. In embodiments where the target protein is CD20, the T cell depleting agent is an anti-CD20 antibody, for example, rituximab.

In further embodiments of any of the aforesaid methods, the methods further include transplanting a cell, for example, a hematopoietic stem cell, or a bone marrow, into the mammal. In some embodiments, the invention features a method of conditioning a mammal prior to cell transplantation. The method includes administering to the mammal an effective amount of the cell comprising a CAR nucleic acid or polypeptide, for example, a CD19 CAR nucleic acid or polypeptide. In some embodiments, the cell transplantation is a stem cell transplantation, for example, a hematopoietic stem cell transplantation, or a bone marrow transplantation. In other embodiments, conditioning a subject prior to cell transplantation includes reducing the number of target-expressing cells in a subject, for example, CD19-expressing normal cells or CD19-expressing cancer cells.

### Dosage Regimen

In some embodiments, a dose of viable CAR-expressing cells (for example, viable CD19, BCMA, CD20, or CD22 CAR-expressing cells) or a pharmaceutical composition comprising said cells comprises about 1×10⁶ to about 1×10⁸ (e.g., about 2×10⁶ to about 5×10⁷, about 5×10⁶ to about 2×10⁷, about 1×10⁶ to about 1×10⁷, about 1×10⁷ to about 1×10⁸, about 1×10⁶ to about 3×10⁶, about 2×10⁶ to about 4×10⁶, about 3×10⁶ to about 5×10⁶, about 4×10⁶ to about 6×10⁶, about 5×10⁶ to about 7×10⁶, about 6×10⁶ to about 8×10⁶, about 7×10⁶ to about 9×10⁶, about 8×10⁶ to about 1×10⁷, about 9×10⁶ to about 2×10⁷, about 1×10⁷ to about 3×10⁷, about 2×10⁷ to about 4×10⁷, about 3×10⁷ to about 5×10⁷, about 4×10⁷ to about 6×10⁷, about 5×10⁷ to about 7×10⁷, about 6×10⁷ to about 8×10⁷, about 7×10⁷ to about 9×10⁷, about 8×10⁷ to about 1×10⁸, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸) CAR-positive viable cells (e.g., BCMA CAR+ T cells). In some embodiments, a dose of viable CAR-expressing cells (for example, viable CD19, BCMA, CD20, or CD22 CAR-expressing cells) comprises about 0.5×10⁶ viable CAR-expressing cells to about 1.25×10⁹ viable CAR-expressing cells (for example, 0.5×10⁶ viable CAR-expressing cells to 1.25×10⁹ viable CAR-expressing cells). In some embodiments, a dose of viable CAR-expressing cells (for example, viable CD19, BCMA, CD20, or CD22 CAR-expressing cells) comprises about 1×10⁶, about 2.5×10⁶, about 5×10⁶, about 1.25×10⁷, about 2.5×10⁷, about 5×10⁷, about 5.75×10⁷, or about 8×10⁷ viable CAR-expressing cells.

In some embodiments, the dose calculation is based on the number of BCMA-CAR+ viable T-cells (e.g., single-positive BCMA CAR cells plus double-positive BCMA+/CD19+ CAR cells), measured by flow cytometry on day 4 (96 h) post-transduction (day 3 (72 h) post-harvest), as described herein. In some embodiments, a dose of viable CAR-expressing cells (for example a BCMA/CD19 dual CART cellular product) comprises about 5×10⁶ to about 2×10⁷ CAR-positive viable cells (e.g., BCMA CAR+ T cells).

In some embodiments, a dose of viable CAR-expressing cells (for example, viable CD19, BCMA, CD20, or CD22 CAR-expressing cells) or a pharmaceutical composition comprising said cells is administered to the subject in one or more (e.g., 2, 3, 4, or more) doses. In some embodiments, the cells or pharmaceutical composition is administered to the subject in two doses. In some embodiments, the one or more doses comprises a first dose and a second dose, wherein the number of CAR-positive viable cells (e.g., BCMA CAR+ T cells) in the first dose is greater than, equal to, or less than the number of CAR-positive viable cells (e.g., BCMA CAR+ T cells) in the second dose.

In some embodiments, the one or more doses comprise a first dose and a second dose, wherein:
(a) the first dose comprises about 1×10⁶ to about 1×10⁷(e.g., about 2×10⁶ to about 8×10⁶, about 4×10⁶ to about 6×10⁶, about 1×10⁶ to about 5×10⁶, about 5×10⁶ to about 1×10⁷, about 1×10⁶ to about 3×10⁶, about 2×10⁶ to about 4×10⁶, about 3×10⁶ to about 5×10⁶, about 4×10⁶ to about 6×10⁶, about 5×10⁶ to about 7×10⁶, about 6×10⁶ to about 8×10⁶, about 7×10⁶ to about 9×10⁶, about 8×10⁶ to about 1×10⁷, about 1×10⁶, about 2×10⁶, about 3×10⁶, about 4×10⁶, about 5×10⁶, about 6×10⁶, about 7×10⁶, about 8×10⁶, about 9×10⁶, or about 1×10⁷) viable CAR-positive cells (e.g., BCMA CAR+ T cells);
(b) the second dose comprises about 1×10⁷ to about 1×10⁸ (e.g., about 2×10⁷ to about 8×10⁷, about 4×10⁷ to about 6×10⁷, about 1×10⁷ to about 5×10⁷, about 5×10⁷ to about 1×10⁸, about 1×10⁷ to about 3×10⁷, about 2×10⁷ to about 4×10⁷, about 3×10⁷ to about 5×10⁷, about 4×10⁷ to about 6×10⁷, about 5×10⁷ to about 7×10⁷, about 6×10⁷ to about 8×10⁷, about 7×10⁷ to about 9×10⁷, about 8×10⁷ to about 1×10⁸, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸) CAR-positive viable cells (e.g., BCMA CAR+ T cells);
(c) the number of CAR-positive viable cells (e.g., BCMA CAR+ T cells) in the first dose is no more than 1/X, wherein X is 1, 2, 3, 4, 5, 6, 7, 8, 9, 10, 20, 30, 40, 50, 60, 70, 80, 90, or 100, of the number of CAR-positive viable cells (e.g., BCMA CAR+ T cells) in the second dose; and/or
(d) the number of CAR-positive viable cells (e.g., BCMA CAR+ T cells) in the first dose is between about 1% and 100% (e.g., between about 10% and about 90%, between about 20% and about 80%, between about 30% and about 70%, between about 40% and about 60%, between about 10% and about 50%, between about 50% and about 90%, between about 10% and about 30%, between about 20% and about 40%, between about 30% and about 50%, between about 50% and about 70%, between about 60% and about 80%, or between about 70% and about 90%) of the number of CAR-positive viable cells (e.g., BCMA CAR+ T cells) in the second dose.

In some embodiments, the first dose comprises about 5×10⁶ viable CAR-positive cells (e.g., BCMA CAR+ T cells). In some embodiments, the second dose comprises about 1×10⁷ or about 2×10⁷ viable CAR-positive cells (e.g., BCMA CAR+ T cells).

In some embodiments, the dose of CAR-positive cells may be increased from the starting dose at a subsequent administration. For example, patient may receive a starting dose of about 1×10⁶ to about 1×10⁷ (e.g., about 5×10⁶) viable CAR-positive cells and may receive a second dose of about 1×10⁷ to about 1×10⁸ (e.g., about 1×10⁷ or about 2×10⁷) CAR-positive viable cells (e.g., BCMA CAR+ T cells).

### Patient Selection

In some embodiments of any of the methods of treating a subject, or composition for use disclosed herein, the subject has a cancer, for example, a hematological cancer. In some embodiments, the cancer is chosen from lymphocytic leukemia (CLL), mantle cell lymphoma (MCL), multiple myeloma, acute lymphoid leukemia (ALL), Hodgkin lymphoma, B-cell acute lymphoid leukemia (BALL), T-cell acute lymphoid leukemia (TALL), small lymphocytic leukemia (SLL), B cell prolymphocytic leukemia, blastic plasmacytoid dendritic cell neoplasm, Burkitt's lymphoma, diffuse large B cell lymphoma (DLBCL), DLBCL associated with chronic inflammation, chronic myeloid leukemia, myeloproliferative neoplasms, follicular lymphoma, pediatric follicular lymphoma, hairy cell leukemia, small cell- or a large cell-follicular lymphoma, malignant lymphoproliferative conditions, MALT lymphoma (extranodal marginal zone lymphoma of mucosa-associated lymphoid tissue), Marginal zone lymphoma, myelodysplasia, myelodysplastic syndrome, non-Hodgkin lymphoma, plasmablastic lymphoma, plasmacytoid dendritic cell neoplasm, Waldenstrom macroglobulinemia, splenic marginal zone lymphoma, splenic lymphoma/leukemia, splenic diffuse red pulp small B-cell lymphoma, hairy cell leukemia-variant, lymphoplasmacytic lymphoma, a heavy chain disease, plasma cell myeloma, solitary plasmocytoma of bone, extraosseous plasmocytoma, nodal marginal zone lymphoma, pediatric nodal marginal zone lymphoma, primary cutaneous follicle center lymphoma, lymphomatoid granulomatosis, primary mediastinal (thymic) large B-cell lymphoma, intravascular large B-cell lymphoma, ALK+ large B-cell lymphoma, large B-cell lymphoma arising in HHV8-associated multicentric Castleman disease, primary effusion lymphoma, B-cell lymphoma, acute myeloid leukemia (AML), or unclassifiable lymphoma. In some embodiments, the cancer is a relapsed and/or refractory cancer.

In some embodiments of any of the methods of treating a subject, or composition for use disclosed herein, the subject has CLL or SLL. In some embodiments, the subject having CLL or SLL has previously been administered a BTK inhibitor therapy, for example, ibrutinib, for least 1-12 months, for example, 6 months. In some embodiments, the BTK inhibitor therapy, for example, ibrutinib therapy, is a second line therapy. In some embodiments, the subject had a partial response, or had stable disease in response to the BTK inhibitor therapy. In some embodiments, the subject did not response to the BTK inhibitor therapy. In some embodiments, the subject developed resistance, for example, developed ibrutinib resistance mutations. In some embodiments, the ibrutinib resistance mutations comprise a mutation in the gene encoding BTK and/or the gene encoding PLCg2. In some embodiments, the subject is an adult, for example, at least 18 years of age.

In some embodiments of any of the methods of treating a subject, or composition for use disclosed herein, the subject has DLBCL, for example, relapsed and/or refractory DLBCL. In some embodiments, the subject having DLBCL, for example, relapsed and/or refractory DLBCL, has previously been administered at least 2 lines of chemotherapy, for example, an anti-CD20 therapy and/or an anthracycline-based chemotherapy. In some embodiments, the subject has previously received stem cell therapy, for example, autologous stem cell therapy, and has not responded to said stem cell therapy. In some embodiments, the subject is not eligible for stem cell therapy, for example, autologous stem cell therapy. In some embodiments, the subject is an adult, for example, at least 18 years of age.

### Therapeutic Application

BCMA Associated Diseases and/or Disorders

In one aspect, the invention provides methods for treating a disease associated with BCMA expression. In one aspect, the invention provides methods for treating a disease wherein part of the tumor is negative for BCMA and part of the tumor is positive for BCMA For example, the CAR of the invention is useful for treating subjects that have undergone treatment for a disease associated with elevated expression of BCMA, wherein the subject that has undergone treatment for elevated levels of BCMA exhibits a disease associated with elevated levels of BCMA. In embodiments, the CAR of the invention is useful for treating subjects that have undergone treatment for a disease associated with expression of BCMA, wherein the subject that has undergone treatment related to expression of BCMA exhibits a disease associated with expression of BCMA.

In one embodiment, the invention provides methods for treating a disease wherein BCMA is expressed on both normal cells and cancers cells, but is expressed at lower levels on normal cells. In one embodiment, the method further comprises selecting a CAR that binds of the invention with an affinity that allows the BCMA CAR to bind and kill the cancer cells expressing BCMA but less than 30%, 25%, 20%, 15%, 10%, 5% or less of the normal cells expressing BCMA are killed, e.g., as determined by an assay described herein. For example, a killing assay such as flow cytometry based on Cr51 CTL can be used. In one embodiment, the BCMA CAR has an antigen binding domain that has a binding affinity KD of 10⁻⁴ M to 10⁻⁵ M, e.g., 10⁻⁶ M to 10⁻⁷ M, e.g., 10⁻⁶ M or 10⁻⁷ M, for the target antigen. In one embodiment, the BCMA antigen binding domain has a binding affinity that is at least five-fold, 10-fold, 20-fold, 30-fold, 50-fold, 100-fold or 1,000-fold less than a reference antibody, e.g., an antibody described herein.

In one aspect, the invention pertains to a vector comprising BCMA CAR operably linked to promoter for expression in mammalian immune effector cells, e.g., T cells or NK cells. In one aspect, the invention provides a recombinant immune effector cell, e.g., T cell or NK cell, expressing the BCMA CAR for use in treating BCMA-expressing tumors, wherein the recombinant immune effector cell (e.g., T cell or NK cell) expressing the BCMA CAR is termed a BCMA CAR-expressing cell (e.g., BCMA CART or BCMA CAR-expressing NK cell). In one aspect, the BCMA CAR-expressing cell (e.g., BCMA CART or BCMA CAR-expressing NK cell) of the invention is capable of contacting a tumor cell with at least one BCMA CAR of the invention expressed on its surface such that the BCMA CAR-expressing cell (e.g., BCMA CART or BCMA CAR-expressing NK cell) targets the tumor cell and growth of the tumor is inhibited.

In one aspect, the invention pertains to a method of inhibiting growth of a BCMA-expressing tumor cell, comprising contacting the tumor cell with a BCMA CAR-expressing cell (e.g., BCMA CART or BCMA CAR-expressing NK cell) of the present invention such that the BCMA CAR-expressing cell (e.g., BCMA CART or BCMA CAR-expressing NK cell) is activated in response to the antigen and targets the cancer cell, wherein the growth of the tumor is inhibited.

In one aspect, the invention pertains to a method of treating cancer in a subject. The method comprises administering to the subject a BCMA CAR-expressing cell (e.g., BCMA CART or BCMA CAR-expressing NK cell) of the present invention such that the cancer is treated in the subject. An example of a cancer that is treatable by the BCMA CAR-expressing cell (e.g., BCMA CART or BCMA CAR-expressing NK cell) of the invention is a cancer associated with expression of BCMA.

The invention includes a type of cellular therapy where immune effector cells (e.g., T cells or NK cells) are genetically modified to express a chimeric antigen receptor (CAR) and the BCMA CAR-expressing cell (e.g., BCMA CART or BCMA CAR-expressing NK cell) is infused to a recipient in need thereof. The infused cell is able to kill tumor cells in the recipient. Unlike antibody therapies, CAR-modified cells, e.g., T cells or NK cells, are able to replicate in vivo resulting in long-term persistence that can lead to sustained tumor control. In various aspects, the cells (e.g., T cells or NK cells) administered to the patient, or their progeny, persist in the patient for at least four months, five months, six months, seven months, eight months, nine months, ten months, eleven months, twelve months, thirteen months, fourteen month, fifteen months, sixteen months, seventeen months, eighteen months, nineteen months, twenty months, twenty-one months, twenty-two months, twenty-three months, two years, three years, four years, or five years after administration of the cell (e.g., T cell or NK cell) to the patient.

The invention also includes a type of cellular therapy where immune effector cells (e.g., T cells or NK cells) are modified, e.g., by in vitro transcribed RNA, to transiently express a chimeric antigen receptor (CAR) and the immune effector cell (e.g., T cell or NK cell) is infused to a recipient in need thereof. The infused cell is able to kill tumor cells in the recipient. Thus, in various aspects, the immune effector cells (e.g., T cells or NK cells) administered to the patient, is present for less than one month, e.g., three weeks, two weeks, one week, after administration of the immune effector cell (e.g., T cell or NK cell) to the patient.

Without wishing to be bound by theory, the anti-tumor immunity response elicited by the CAR-modified immune effector cells (e.g., T cells or NK cells) may be an active or a passive immune response, or alternatively may be due to a direct vs indirect immune response. In one aspect, the CAR transduced immune effector cells (e.g., T cells or NK cells) exhibit specific proinflammatory cytokine secretion and potent cytolytic activity in response to human cancer cells expressing the BCMA, resist soluble BCMA inhibition, mediate bystander killing and mediate regression of an established human tumor. For example, antigen-less tumor cells within a heterogeneous field of BCMA-expressing tumor may be susceptible to indirect destruction by BCMA-redirected immune effector cells (e.g., T cells or NK cells) that has previously reacted against adjacent antigen-positive cancer cells.

In one aspect, the fully-human CAR-modified immune effector cells (e.g., T cells or NK cells) of the invention may be a type of vaccine for ex vivo immunization and/or in vivo therapy in a mammal. In one aspect, the mammal is a human.

With respect to ex vivo immunization, at least one of the following occurs in vitro prior to administering the cell into a mammal: i) expansion of the cells, ii) introducing a nucleic acid encoding a CAR to the cells or iii) cryopreservation of the cells.

Ex vivo procedures are well known in the art and are discussed more fully below. Briefly, cells are isolated from a mammal (e.g., a human) and genetically modified (i.e., transduced or transfected in vitro) with a vector expressing a CAR disclosed herein. The CAR-modified cell can be administered to a mammalian recipient to provide a therapeutic benefit. The mammalian recipient may be a human and the CAR-modified cell can be autologous with respect to the recipient. Alternatively, the cells can be allogeneic, syngeneic or xenogeneic with respect to the recipient.

The procedure for ex vivo expansion of hematopoietic stem and progenitor cells is described in U.S. Pat. No. 5,199,942, incorporated herein by reference, can be applied to the cells of the present invention. Other suitable methods are known in the art, therefore the present invention is not limited to any particular method of ex vivo expansion of the cells. Briefly, ex vivo culture and expansion of T cells comprises: (1) collecting CD34+ hematopoietic stem and progenitor cells from a mammal from peripheral blood harvest or bone marrow explants; and (2) expanding such cells ex vivo. In addition to the cellular growth factors described in U.S. Pat. No. 5,199,942, other factors such as flt3-L, IL-1, IL-3 and c-kit ligand, can be used for culturing and expansion of the cells.

In addition to using a cell-based vaccine in terms of ex vivo immunization, the present invention also provides compositions and methods for in vivo immunization to elicit an immune response directed against an antigen in a patient.

Generally, the cells activated and expanded as described herein may be utilized in the treatment and prevention of diseases that arise in individuals who are immunocompromised. For example, the CAR-modified immune effector cells (e.g., T cells or NK cells) of the invention are used in the treatment of diseases, disorders and conditions associated with expression of BCMA. In some aspects, the cells of the invention are used in the treatment of patients at risk for developing diseases, disorders and conditions associated with expression of BCMA. Thus, the present invention provides methods for the treatment or prevention of diseases, disorders and conditions associated with expression of BCMA comprising administering to a subject in need thereof, a therapeutically effective amount of the CAR-modified immune effector cells (e.g., T cells or NK cells) of the invention.

In one aspect the CAR-expressing cells (e.g., CART cells or CAR-expressing NK cells) of the inventions may be used to treat a proliferative disease such as a cancer or malignancy or is a precancerous condition such as a myelodysplasia, a myelodysplastic syndrome or a preleukemia. In one aspect, the cancer is a hematological cancer. Hematological cancer conditions are the types of cancer such as leukemia and malignant lymphoproliferative conditions that affect blood, bone marrow and the lymphatic system. In one aspect, the hematological cancer is a leukemia or a hematological. An example of a disease or disorder associated with BCMA is multiple myeloma (also known as MM) (See Claudio et al., Blood. 2002, 100(6):2175-86; and Novak et al., Blood. 2004, 103(2):689-94). Multiple myeloma, also known as plasma cell myeloma or Kahler's disease, is a cancer characterized by an accumulation of abnormal or malignant plasma B-cells in the bone marrow. Frequently, the cancer cells invade adjacent bone, destroying skeletal structures and resulting in bone pain and fractures. Most cases of myeloma also features the production of a paraprotein (also known as M proteins or myeloma proteins), which is an abnormal immunoglobulin produced in excess by the clonal proliferation of the malignant plasma cells. Blood serum paraprotein levels of more than 30 g/L is diagnostic of multiple myeloma, according to the diagnostic criteria of the International Myeloma Working Group (IMWG) (See Kyle et al. (2009), Leukemia. 23:3-9). Other symptoms or signs of multiple myeloma include reduced kidney function or renal failure, bone lesions, anemia, hypercalcemia, and neurological symptoms.

Criteria for distinguishing multiple myeloma from other plasma cell proliferative disorders have been established by the International Myeloma Working Group (See Kyle et al. (2009), Leukemia. 23:3-9). All three of the following criteria must be met:
Clonal bone marrow plasma cells ≥10%
Present of serum and/or urinary monoclonal protein (except in patients with true non-secretory multiple myeloma)
Evidence of end-organ damage attributable to the underlying plasma cell proliferative disorder, specifically:
   Hypercalcemia: serum calcium ≥11.5 mg/100 ml
   Renal insufficiency: serum creatinine >1.73 mmol/l
   Anemia: normochromic, normocytic with a hemoglobin value of
>2 g/100 ml below the lower limit of normal, or a hemoglobin value <10 g/100 ml
   Bone lesions: lytic lesions, severe osteopenia, or pathologic fractures.

Other plasma cell proliferative disorders that can be treated by the compositions and methods described herein include, but are not limited to, asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

Two staging systems are used in the staging of multiple myeloma: the International Staging System (ISS) (See Greipp et al. (2005), J. Clin. Oncol. 23 (15):3412-3420) and the Durie-Salmon Staging system (DSS) (See Durie et al. (1975), Cancer 36 (3): 842-854).

A third staging system for multiple myeloma is referred to as Revised International Staging System (R-ISS) (see Palumbo A, Avet-Loiseau H, Oliva S, et al. Journal of clinical oncology: official journal of the American Society of Clinical Oncology 2015; 33:2863-9, herein incorporated by reference in its entirety). R-ISS stage I includes ISS stage I (serum β2-microglobulin level <3.5 mg/L and serum albumin level ≥3.5 g/dL), no high-risk CA [del(17p) and/or t(4; 14) and/or t(14; 16)], and normal LDH level (less than the upper limit of normal range). R-ISS stage III includes ISS stage III (serum 02-microglobulin level >5.5 mg/L) and high-risk CA or high LDH level. R-ISS stage II includes all the other possible combinations.

The response of patients can be determined based on IMWG 2016 criteria, as disclosed in Kumar S, Paiva B, Anderson K C, et al. International Myeloma Working Group consensus criteria for response and minimal residual disease assessment in multiple myeloma. The Lancet Oncology; 17(8):e328-e346, herein incorporated by reference in its entirety.

Standard treatment for multiple myeloma and associated diseases includes chemotherapy, stem cell transplant (autologous or allogeneic), radiation therapy, and other drug therapies. Frequently used anti-myeloma drugs include alkylating agents (e.g., bendamustine, cyclophosphamide and melphalan), proteasome inhibitors (e.g., bortezomib), corticosteroids (e.g., dexamethasone and prednisone), and immunomodulators (e.g., thalidomide and lenalidomide or Revlimid®), or any combination thereof. Biphosphonate drugs are also frequently administered in combination with the standard anti-MM treatments to prevent bone loss. Patients older than 65-70 years of age are unlikely candidates for stem cell transplant. In some cases, double-autologous stem cell transplants are options for patients less than 60 years of age with suboptimal response to the first transplant. The compositions and methods of the present invention may be administered in combination with any of the currently prescribed treatments for multiple myeloma.

Another example of a disease or disorder associated with BCMA is Hodgkin's lymphoma and non-Hodgkin's lymphoma (See Chiu et al., Blood. 2007, 109(2):729-39; He et al., J Immunol. 2004, 172(5):3268-79).

Hodgkin's lymphoma (HL), also known as Hodgkin's disease, is a cancer of the lymphatic system that originates from white blood cells, or lymphocytes. The abnormal cells that comprise the lymphoma are called Reed-Sternberg cells. In Hodgkin's lymphoma, the cancer spreads from one lymph node group to another. Hodgkin's lymphoma can be subclassified into four pathologic subtypes based upon Reed-Sternberg cell morphology and the cell composition around the Reed-Sternberg cells (as determined through lymph node biopsy): nodular sclerosing HL, mixed-cellularity subtype, lymphocyte-rich or lymphocytic predominance, lymphocyte depleted. Some Hodgkin's lymphoma can also be nodular lymphocyte predominant Hodgkin's lymphoma, or can be unspecified. Symptoms and signs of Hodgkin's lymphoma include painless swelling in the lymph nodes in the neck, armpits, or groin, fever, night sweats, weight loss, fatigue, itching, or abdominal pain.

Non-Hodgkin's lymphoma (NHL) comprises a diverse group of blood cancers that include any kind of lymphoma other than Hodgkin's lymphoma. Subtypes of non-Hodgkin's lymphoma are classified primarily by cell morphology, chromosomal aberrations, and surface markers. NHL subtypes (or NHL-associated cancers) include B cell lymphomas such as, but not limited to, Burkitt's lymphoma, B-cell chronic lymphocytic leukemia (B-CLL), B-cell prolymphocytic leukemia (B-PLL), chronic lymphocytic leukemia (CLL), diffuse large B-cell lymphoma (DLBCL) (e.g., intravascular large B-cell lymphoma and primary mediastinal B-cell lymphoma), follicular lymphoma (e.g., follicle center lymphoma, follicular small cleaved cell), hair cell leukemia, high grade B-cell lymphoma (Burkitt's like), lymphoplasmacytic lymphoma (Waldenstrom's macroglobulinemia), mantle cell lymphoma, marginal zone B-cell lymphomas (e.g., extranodal marginal zone B-cell lymphoma or mucosa-associated lymphoid tissue (MALT) lymphoma, nodal marginal zone B-cell lymphoma, and splenic marginal zone B-cell lymphoma), plasmacytoma/myeloma, precursor B-lymphoblastic leukemia/lymphoma (PB-LBL/L), primary central nervous system (CNS) lymphoma, primary intraocular lymphoma, small lymphocytic lymphoma (SLL); and T cell lymphomas, such as, but not limited to, anaplastic large cell lymphoma (ALCL), adult T-cell lymphoma/leukemia (e.g., smoldering, chronic, acute and lymphomatous), angiocentric lymphoma, angioimmunoblastic T-cell lymphoma, cutaneous T-cell lymphomas (e.g., mycosis fungoides, Sezary syndrome, etc.), extranodal natural killer/T-cell lymphoma (nasal-type), enteropathy type intestinal T-cell lymphoma, large granular lymphocyte leukemia, precursor T-lymphoblastic lymphoma/leukemia (T-LBL/L), T-cell chronic lymphocytic leukemia/prolymphocytic leukemia (T-CLL/PLL), and unspecified peripheral T-cell lymphoma. Symptoms and signs of Hodgkin's lymphoma include painless swelling in the lymph nodes in the neck, armpits, or groin, fever, night sweats, weight loss, fatigue, itching, abdominal pain, coughing, or chest pain.

The staging is the same for both Hodgkin's and non-Hodgkin's lymphoma, and refers to the extent of spread of the cancer cells within the body. In stage I, the lymphoma cells are in one lymph node group. In stage II, lymphoma cells are present in at least two lymph node groups, but both groups are on the same side of the diaphragm, or in one part of a tissue or organ and the lymph nodes near that organ on the same side of the diaphragm. In stage III, lymphoma cells are in lymph nodes on both sides of the diaphragm, or in one part of a tissue or organ near these lymph node groups or in the spleen. In stage IV, lymphoma cells are found in several parts of at least one organ or tissue, or lymphoma cells are in an organ and in lymph nodes on the other side of the diaphragm. In addition to the Roman numeral staging designation, the stages of can also be described by letters A, B, E, and S, wherein A refers to patients without symptoms, B refers to patients with symptoms, E refers to patients in which lymphoma is found in tissues outside the lymph system, and S refers to patients in which lymphoma is found in the spleen.

Hodgkin's lymphoma is commonly treated with radiation therapy, chemotherapy, or hematopoietic stem cell transplantation. The most common therapy for non-Hodgkin's lymphoma is R-CHOP, which consists of four different chemotherapies (cyclophosphamide, doxorubicin, vincristine, and prednisolone) and rituximab (Rituxan®). Other therapies commonly used to treat NHL include other chemotherapeutic agents, radiation therapy, stem cell transplantation (autologous or allogeneic bone marrow transplantation), or biological therapy, such as immunotherapy. Other examples of biological therapeutic agents include, but are not limited to, rituximab (Rituxan®), tositumomab (Bexxar®), epratuzumab (LymphoCide®), and alemtuzumab (MabCampath®). The compositions and methods of the present invention may be administered in combination with any of the currently prescribed treatments for Hodgkin's lymphoma or non-Hodgkin's lymphoma.

BCMA expression has also been associated Waldenstrom's macroglobulinemia (WM), also known as lymphoplasmacytic lymphoma (LPL). (See Elsawa et al., Blood. 2006, 107(7):2882-8). Waldenstrom's macroglobulinemia was previously considered to be related to multiple myeloma, but has more recently been classified as a subtype of non-Hodgkin's lymphoma. WM is characterized by uncontrolled B-cell lymphocyte proliferation, resulting in anemia and production of excess amounts of paraprotein, or immunoglobulin M (IgM), which thickens the blood and results in hyperviscosity syndrome. Other symptoms or signs of WM include fever, night sweats, fatigue, anemia, weight loss, lymphadenopathy or splenomegaly, blurred vision, dizziness, nose bleeds, bleeding gums, unusual bruises, renal impairment or failure, amyloidosis, or peripheral neuropathy.

Standard treatment for WM consists of chemotherapy, specifically with rituximab (Rituxan®). Other chemotherapeutic drugs can be used in combination, such as chlorambucil (Leukeran®), cyclophosphamide (Neosar®), fludarabine (Fludara®), cladribine (Leustatin®), vincristine, and/or thalidomide. Corticosteroids, such as prednisone, can also be administered in combination with the chemotherapy. Plasmapheresis, or plasma exchange, is commonly used throughout treatment of the patient to alleviate some symptoms by removing the paraprotein from the blood. In some cases, stem cell transplantation is an option for some patients.

Another example of a disease or disorder associated with BCMA is brain cancer. Specifically, expression of BCMA has been associated with astrocytoma or glioblastoma (See Deshayes et al, Oncogene. 2004, 23(17):3005-12, Pelekanou et al., PLoS One. 2013, 8(12):e83250). Astrocytomas are tumors that arise from astrocytes, which are a type of glial cell in the brain. Glioblastoma (also known as glioblastoma multiforme or GBM) is the most malignant form of astrocytoma, and is considered the most advanced stage of brain cancer (stage IV). There are two variants of glioblastoma: giant cell glioblastoma and gliosarcoma. Other astrocytomas include juvenile pilocytic astrocytoma (JPA), fibrillary astrocytoma, pleomorphic xanthoastrocytoma (PXA), dysembryoplastic neuroepithelial tumor (DNET), and anaplastic astrocytoma (AA).

Symptoms or signs associated with glioblastoma or astrocytoma include increased pressure in the brain, headaches, seizures, memory loss, changes in behavior, loss in movement or sensation on one side of the body, language dysfunction, cognitive impairments, visual impairment, nausea, vomiting, and weakness in the arms or legs.

Surgical removal of the tumor (or resection) is the standard treatment for removal of as much of the glioma as possible without damaging or with minimal damage to the normal, surrounding brain. Radiation therapy and/or chemotherapy are often used after surgery to suppress and slow recurrent disease from any remaining cancer cells or satellite lesions. Radiation therapy includes whole brain radiotherapy (conventional external beam radiation), targeted three-dimensional conformal radiotherapy, and targeted radionuclides. Chemotherapeutic agents commonly used to treat glioblastoma include temozolomide, gefitinib or erlotinib, and cisplatin. Angiogenesis inhibitors, such as Bevacizumab (Avastin®), are also commonly used in combination with chemotherapy and/or radiotherapy.

Supportive treatment is also frequently used to relieve neurological symptoms and improve neurologic function, and is administered in combination any of the cancer therapies described herein. The primary supportive agents include anticonvulsants and corticosteroids. Thus, the compositions and methods of the present invention may be used in combination with any of the standard or supportive treatments to treat a glioblastoma or astrocytoma.

Non-cancer related diseases and disorders associated with BCMA expression can also be treated by the compositions and methods disclosed herein. Examples of non-cancer related diseases and disorders associated with BCMA expression include, but are not limited to: viral infections; e.g., HIV, fungal infections, e.g., C. neoformans; irritable bowel disease; ulcerative colitis, and disorders related to mucosal immunity.

The CAR-modified immune effector cells (e.g., T cells or NK cells) of the present invention may be administered either alone, or as a pharmaceutical composition in combination with diluents and/or with other components such as IL-2 or other cytokines or cell populations.

The present invention provides for compositions and methods for treating cancer. In one aspect, the cancer is a hematologic cancer including but is not limited to hematological cancer is a leukemia or a lymphoma. In one aspect, the CAR-expressing cells (e.g., CART cells or CAR-expressing NK cells) of the invention may be used to treat cancers and malignancies such as, but not limited to, e.g., acute leukemias including but not limited to, e.g., B-cell acute lymphoid leukemia (“BALL”), T-cell acute lymphoid leukemia (“TALL”), acute lymphoid leukemia (ALL); one or more chronic leukemias including but not limited to, e.g., chronic myelogenous leukemia (CML), chronic lymphocytic leukemia (CLL); additional hematologic cancers or hematologic conditions including, but not limited to, e.g., B cell prolymphocytic leukemia, blastic plasmacytoid dendritic cell neoplasm, Burkitt's lymphoma, diffuse large B cell lymphoma, Follicular lymphoma, Hairy cell leukemia, small cell- or a large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, and “preleukemia” which are a diverse collection of hematological conditions united by ineffective production (or dysplasia) of myeloid blood cells, and the like. Further a disease associated with BCMA expression includes, but not limited to, e.g., atypical and/or non-classical cancers, malignancies, precancerous conditions or proliferative diseases expressing BCMA.

In embodiments, a composition described herein can be used to treat a disease including but not limited to a plasma cell proliferative disorder, e.g., asymptomatic myeloma (smoldering multiple myeloma or indolent myeloma), monoclonal gammapathy of undetermined significance (MGUS), Waldenstrom's macroglobulinemia, plasmacytomas (e.g., plasma cell dyscrasia, solitary myeloma, solitary plasmacytoma, extramedullary plasmacytoma, and multiple plasmacytoma), systemic amyloid light chain amyloidosis, and POEMS syndrome (also known as Crow-Fukase syndrome, Takatsuki disease, and PEP syndrome).

In embodiments, a composition described herein can be used to treat a disease including but not limited to a cancer, e.g., a cancer described herein, e.g., a prostate cancer (e.g., castrate-resistant or therapy-resistant prostate cancer, or metastatic prostate cancer), pancreatic cancer, or lung cancer.

The present invention also provides methods for inhibiting the proliferation or reducing a BCMA-expressing cell population, the methods comprising contacting a population of cells comprising a BMCA-expressing cell with an anti-BCMA CAR-expressing cell (e.g., BCMA CART cell or BCMA CAR-expressing NK cell) of the invention that binds to the BCMA-expressing cell. In a specific aspect, the present invention provides methods for inhibiting the proliferation or reducing the population of cancer cells expressing BCMA, the methods comprising contacting the BCMA-expressing cancer cell population with an anti-BCMA CAR-expressing cell (e.g., BCMA CART cell or BCMA CAR-expressing NK cell) of the invention that binds to the BCMA-expressing cell. In one aspect, the present invention provides methods for inhibiting the proliferation or reducing the population of cancer cells expressing BCMA, the methods comprising contacting the BMCA-expressing cancer cell population with an anti-BCMA CAR-expressing cell (e.g., BCMA CART cell or BCMA CAR-expressing NK cell) of the invention that binds to the BCMA-expressing cell. In some aspects, the anti-BCMA CAR-expressing cell (e.g., BCMA CART cell or BCMA CAR-expressing NK cell) of the invention reduces the quantity, number, amount or percentage of cells and/or cancer cells by at least 25%, at least 30%, at least 40%, at least 50%, at least 65%, at least 75%, at least 85%, at least 95%, or at least 99% in a subject with or animal model for myeloid leukemia or another cancer associated with BCMA-expressing cells relative to a negative control. In one aspect, the subject is a human.

The present invention also provides methods for preventing, treating and/or managing a disease associated with BCMA-expressing cells (e.g., a hematologic cancer or atypical cancer expressing BCMA), the methods comprising administering to a subject in need an anti-BCMA CAR-expressing cell (e.g., BCMA CART cell or BCMA CAR-expressing NK cell) of the invention that binds to the BCMA-expressing cell. In one aspect, the subject is a human. Non-limiting examples of disorders associated with BCMA-expressing cells include viral or fungal infections, and disorders related to mucosal immunity.

The present invention also provides methods for preventing, treating and/or managing a disease associated with BCMA-expressing cells, the methods comprising administering to a subject in need an anti-BCMA CAR-expressing cell (e.g., BCMA CART cell or BCMA CAR-expressing NK cell) of the invention that binds to the BCMA-expressing cell. In one aspect, the subject is a human.

The present invention provides methods for preventing relapse of cancer associated with BCMA-expressing cells, the methods comprising administering to a subject in need thereof an anti-BCMA CAR-expressing cell (e.g., BCMA CART cell or BCMA CAR-expressing NK cell) of the invention that binds to the BCMA-expressing cell. In one aspect, the methods comprise administering to the subject in need thereof an effective amount of an anti-BCMA CAR-expressing cell (e.g., BCMA CART cell or BCMA CAR-expressing NK cell)described herein that binds to the BCMA-expressing cell in combination with an effective amount of another therapy.

### Combination Therapies

A CAR-expressing cell described herein may be used in combination with other known agents and therapies. A CAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed. The CAR therapy and/or other therapeutic agents, procedures or modalities can be administered during periods of active disorder, or during a period of remission or less active disease. The CAR therapy can be administered before the other treatment, concurrently with the treatment, post-treatment, or during remission of the disorder. When administered in combination, the CAR therapy and the additional agent (e.g., second or third agent), or all, can be administered in an amount or dose that is higher, lower or the same than the amount or dosage of each agent used individually, e.g., as a monotherapy. In some embodiments, the administered amount or dosage of the CAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect. In further aspects, a CAR-expressing cell described herein may be used in a treatment regimen in combination with surgery, chemotherapy, radiation, immunosuppressive agents. Exemplary agents and therapies that can be used in combination with a CAR-expressing cell described herein are disclosed on pages 266-313 of WO2016164731, herein incorporated by reference in its entirety.

### Biomarkers for Evaluating CAR-Effectiveness

In some embodiments, disclosed herein is a method of evaluating or monitoring the effectiveness of a CAR-expressing cell therapy (for example, a CD19 or BCMA CAR therapy), in a subject (for example, a subject having a cancer, for example, a hematological cancer). The method includes acquiring a value of effectiveness to the CAR therapy, wherein said value is indicative of the effectiveness or suitability of the CAR-expressing cell therapy.

In embodiments, the value of effectiveness to the CAR therapy in a subject having CLL or SLL, comprises a measure of one, two, three, or all of the following parameters:
(i) a mutation in a gene encoding BTK in a sample (for example, an apheresis sample or a manufactured CAR-expressing cell product sample);
(ii) a mutation in a gene encoding PLCg2 in a sample (for example, an apheresis sample or a manufactured CAR-expressing cell product sample);
(iii) minimal residual disease, for example, as evaluated by the level and/or activity of CD8, CD4, CD3, CD5, CD19, CD20, CD22, CD43, CD79b, CD27, CD45RO, CD45RA, CCR7, CD95, Lag3, PD-1, Tim-3, and/or CD81; or as evaluated by immunoglobulin deep sequencing; in a sample (for example, an apheresis sample or tumor sample from the subject); or
(iv) the level or activity of one, two, three, four, five, six, seven, eight, nine, ten or all of the cytokines chosen from IFN-g, IL-2, IL-4, IL-6, IL-8, IL-10, IL-15, TNF-α, IP-10, MCP1, MIP1a, in a sample, for example, an apheresis sample from the subject.

In embodiments, the value of effectiveness to the CAR therapy in a subject having DLBCL, for example, relapsed and/or refractory DLBCL, comprises a measure of one or both the following parameters:
(i) minimal residual disease, for example, as evaluated by the level and/or activity of CD8, CD4, CAR19, CD3, CD27, CD45RO, CD45RA, CCR7, CD95, Lag3, PD-1, and/or Tim-3; or as evaluated by immunoglobulin deep sequencing; in a sample (for example, an apheresis sample or tumor sample from the subject); or
(ii) the level or activity of one, two, three, four, five, six, seven, eight, nine, ten or all of the cytokines chosen from IFN-g, IL-2, IL-4, IL-6, IL-8, IL-10, IL-15, TNF-α, IP-10, MCP1, MIP1a, in a sample (for example, an apheresis sample from the subject).

In other embodiments, the value of effectiveness to the CAR therapy, further comprises a measure of one, two, three, four, five, six or more (all) of the following parameters:
(i) the level or activity of one, two, three, or more (for example, all) of resting T_{EFF} cells, resting T_{REG} cells, younger T cells (for example, naïve T cells (for example, naïve CD4 or CD8 T cells, naïve gamma/delta T cells), or stem memory T cells (for example, stem memory CD4 or CD8 T cells, or stem memory gamma/delta T cells), or early memory T cells, or a combination thereof, in a sample (for example, an apheresis sample or a manufactured CAR-expressing cell product sample);
(ii) the level or activity of one, two, three, or more (for example, all) of activated T_{EFF} cells, activated T_{REG} cells, older T cells (for example, older CD4 or CD8 cells), or late memory T cells, or a combination thereof, in a sample (for example, an apheresis sample or a manufactured CAR-expressing cell product sample);
(iii) the level or activity of an immune cell exhaustion marker, for example, one, two or more immune checkpoint inhibitors (for example, PD-1, PD-L1, TIM-3, TIGIT and/or LAG-3) in a sample (for example, an apheresis sample or a manufactured CAR-expressing cell product sample). In some embodiments, an immune cell has an exhausted phenotype, for example, co-expresses at least two exhaustion markers, for example, co-expresses PD-1 and TIM-3. In other embodiments, an immune cell has an exhausted phenotype, for example, co-expresses at least two exhaustion markers, for example, co-expresses PD-1 and LAG-3;
(iv) the level or activity of CD27 and/or CD45RO− (for example, CD27+CD45RO−) immune effector cells, for example, in a CD4+ or a CD8+ T cell population, in a sample (for example, an apheresis sample or a manufactured CAR-expressing cell product sample);
(v) the level or activity of one, two, three, four, five, six, seven, eight, nine, ten, eleven or all of the biomarkers chosen from CCL20, IL-17a, IL-6, PD-1, PD-L1, LAG-3, TIM-3, CD57, CD27, CD122, CD62L, KLRG1;
(vi) a cytokine level or activity (for example, quality of cytokine repertoire) in a CAR-expressing cell product sample, for example, CLL-1-expressing cell product sample; or
(vii) a transduction efficiency of a CAR-expressing cell in a manufactured CAR-expressing cell product sample.

In some embodiments of any of the methods disclosed herein, the CAR-expressing cell therapy comprises a plurality (for example, a population) of CAR-expressing immune effector cells, for example, a plurality (for example, a population) of T cells or NK cells, or a combination thereof. In some embodiments, the CAR-expressing cell therapy is a CD19 CAR therapy.

In some embodiments of any of the methods disclosed herein, the measure of one or more of the parameters disclosed herein is obtained from an apheresis sample acquired from the subject. The apheresis sample can be evaluated prior to infusion or re-infusion.

In some embodiments of any of the methods disclosed herein, the measure of one or more of the parameters disclosed herein is obtained from a tumor sample acquired from the subject.

In some embodiments of any of the methods disclosed herein, the measure of one or more of the parameters disclosed herein is obtained from a manufactured CAR-expressing cell product sample, for example, CD19 CAR-expressing cell product sample. The manufactured CAR-expressing cell product can be evaluated prior to infusion or re-infusion.

In some embodiments of any of the methods disclosed herein, the subject is evaluated prior to receiving, during, or after receiving, the CAR-expressing cell therapy.

In some embodiments of any of the methods disclosed herein, the measure of one or more of the parameters disclosed herein evaluates a profile for one or more of gene expression, flow cytometry or protein expression.

In some embodiments of any of the methods disclosed herein, the method further comprises identifying the subject as a responder, a non-responder, a relapser or a non-relapser, based on a measure of one or more of the parameters disclosed herein.

In some embodiments of any of the methods disclosed herein, a responder, for example, complete responder has, or is identified as having, a greater, for example, a statistically significant greater, percentage of CD8+ T cells compared to a reference value, for example, a non-responder percentage of CD8+ T cells.

In some embodiments of any of the methods disclosed herein, a responder, for example, complete responder has, or is identified as having, a greater percentage of CD27+CD45RO− immune effector cells, for example, in the CD8+ population, compared to a reference value, for example, a non-responder number of CD27+CD45RO− immune effector cells.

In some embodiments of any of the methods disclosed herein, a responder, for example, complete responder or a partial responder has, or is identified as having, a greater, for example, a statistically significant greater, percentage of CD4+ T cells compared to a reference value, for example, a non-responder percentage of CD4+ T cells.

In some embodiments of any of the methods disclosed herein, a responder, for example, complete responder has, or is identified as having, a greater percentage of one, two, three, or more (for example, all) of resting T_{EFF} cells, resting T_{REG} cells, younger T cells, or early memory T cells, or a combination thereof, compared to a reference value, for example, a non-responder number of resting T_{EFF} cells, resting T_{REG} cells, younger T cells, or early memory T cells.

In some embodiments of any of the methods disclosed herein, a non-responder has, or is identified as having, a greater percentage of one, two, three, or more (for example, all) of activated T_{EFF} cells, activated T_{REG} cells, older T cells (for example, older CD4 or CD8 cells), or late memory T cells, or a combination thereof, compared to a reference value, for example, a responder number of activated T_{EFF} cells, activated T_{REG} cells, older T cells (for example, older CD4 or CD8 cells), or late memory T cells.

In some embodiments of any of the methods disclosed herein, a non-responder has, or is identified as having, a greater percentage of an immune cell exhaustion marker, for example, one, two or more immune checkpoint inhibitors (for example, PD-1, PD-L1, TIM-3, TIGIT, and/or LAG-3). In some embodiments, a non-responder has, or is identified as having, a greater percentage of PD-1, PD-L1, or LAG-3 expressing immune effector cells (for example, CD4+ T cells and/or CD8+ T cells) (for example, CAR-expressing CD4+ cells and/or CD8+ T cells) compared to the percentage of PD-1 or LAG-3 expressing immune effector cells from a responder.

In some embodiments, a non-responder has, or is identified as having, a greater percentage of immune cells having an exhausted phenotype, for example, immune cells that co-express at least two exhaustion markers, for example, co-expresses PD-1, PD-L1 and/or TIM-3. In other embodiments, a non-responder has, or is identified as having, a greater percentage of immune cells having an exhausted phenotype, for example, immune cells that co-express at least two exhaustion markers, for example, co-expresses PD-1 and LAG-3.

In some embodiments of any of the methods disclosed herein, a non-responder has, or is identified as having, a greater percentage of PD-1/PD-L1+/LAG-3+ cells in the CAR-expressing cell population (for example, a CLL-1 CAR+ cell population) compared to a responder (for example, a complete responder) to the CAR-expressing cell therapy.

In some embodiments of any of the methods disclosed herein, the responder (for example, the complete or partial responder) has one, two, three or more (or all) of the following profile:
(i) has a greater number of CD27+ immune effector cells compared to a reference value, for example, a non-responder number of CD27+ immune effector cells;
(ii) has a greater number of CD8+ T cells compared to a reference value, for example, a non-responder number of CD8+ T cells;
(iii) has a lower number of immune cells expressing one or more checkpoint inhibitors, for example, a checkpoint inhibitor chosen from PD-1, PD-L1, LAG-3, TIM-3, or KLRG-1, or a combination, compared to a reference value, for example, a non-responder number of cells expressing one or more checkpoint inhibitors; or
(iv) has a greater number of one, two, three, four or more (all) of resting T_{EFF} cells, resting T_{REG} cells, naïve CD4 cells, unstimulated memory cells or early memory T cells, or a combination thereof, compared to a reference value, for example, a non-responder number of resting T_{EFF} cells, resting T_{REG} cells, naïve CD4 cells, unstimulated memory cells or early memory T cells.

In embodiments, a subject who is a responder, a non-responder, a relapser or a non-relapser identified by the methods herein can be further evaluated according to clinical criteria. For example, a complete responder has, or is identified as, a subject having a disease, for example, a cancer, who exhibits a complete response, for example, a complete remission, to a treatment. A complete response may be identified, for example, using the NCCN Guidelines®, or the International Workshop on Chronic Lymphocytic Leukemia (iwCLL) 2018 guidelines as disclosed in Hallek M et al., Blood (2018) 131:2745-2760 “iwCLL guidelines for diagnosis, indications for treatment, response assessment, and supportive management of CLL,” the entire contents of which are hereby incorporated by reference in its entirety. A partial responder has, or is identified as, a subject having a disease, for example, a cancer, who exhibits a partial response, for example, a partial remission, to a treatment. A partial response may be identified, for example, using the NCCN Guidelines®, or iwCLL 2018 criteria as described herein. A non-responder has, or is identified as, a subject having a disease, for example, a cancer, who does not exhibit a response to a treatment, for example, the patient has stable disease or progressive disease. A non-responder may be identified, for example, using the NCCN Guidelines®, or iwCLL 2018 criteria as described herein.

Alternatively, or in combination with the methods disclosed herein, responsive to said value, performing one, two, three four or more of:
administering for example, to a responder or a non-relapser, a CAR-expressing cell therapy;
administered an altered dosing of a CAR-expressing cell therapy;
altering the schedule or time course of a CAR-expressing cell therapy;
administering, for example, to a non-responder or a partial responder, an additional agent in combination with a CAR-expressing cell therapy, for example, a checkpoint inhibitor, for example, a checkpoint inhibitor described herein;
administering to a non-responder or partial responder a therapy that increases the number of younger T cells in the subject prior to treatment with a CAR-expressing cell therapy;
modifying a manufacturing process of a CAR-expressing cell therapy, for example, enriching for younger T cells prior to introducing a nucleic acid encoding a CAR, or increasing the transduction efficiency, for example, for a subject identified as a non-responder or a partial responder;
administering an alternative therapy, for example, for a non-responder or partial responder or relapser; or
if the subject is, or is identified as, a non-responder or a relapser, decreasing the T_{REG} cell population and/or T_{REG} gene signature, for example, by one or more of CD25 depletion, administration of cyclophosphamide, anti-GITR antibody, or a combination thereof.

### EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1: In Vitro Characterization of Human BCMA CARs

A set of fully human single chain variable fragments (scFv) was cloned into lentiviral CAR expression vectors with the CD3zeta chain and the 4-1BB stimulatory molecules: R1B6, R1F2, R1G5, P161, B61-10, B61-02, Hy03, and Hy52. The constructs were initially screened using automated cell reporter assay followed by selection for optimal clones based on expression on primary T cells as well as quantity and quality of effector T cell responses (“BCMA CART” or “BCMA CAR T cells”) in response to BCMA expressing (“BCMA+” or “BCMA positive”) targets. Effector T cell responses include, but are not limited to, cellular expansion, proliferation, doubling, cytokine production and target cell killing or cytolytic activity (degranulation).

### Generation of BCMA CAR Lentivirus

All the above-mentioned scFv encoding lentiviral transfer vectors were used to produce the genomic material packaged into the VSVg pseudotyped lentiviral particles. Lentiviral transfer vector DNA encoding the CAR was mixed with the three packaging components VSVg, gag/pol and rev in combination with lipofectamine reagent to transfect Lenti-X 293 T cells (Clontech), followed by medium replacement 12-18 h later. 30 hours after medium change, the media was collected, filtered and stored at −80° C.

### BCMA CAR JNL and JNL Screening Reporter Assay Using Automated System

For the reporter assay, lentivirus encoding for BCMA CARs was generated in HEK293 cells at two different cell densities (40,000 cells (1×H293) or 80,000 cells (2×H293)) in an automated, small scale fashion in 96-well plates, where virus-containing supernatant was harvested 48 h after transfection and used fresh, without freezing, for the transduction of a Jurkat T cell reporter cell line. The Jurkat NFAT Luciferase (JNL) reporter cell line is based on the acute T cell leukemia line Jurkat. The line was modified to express luciferase under control of the Nuclear Factor of Activated T cells (NFAT) response element. For the transduction with BCMA CARs, 10,000 JNL cells/well of a 96-well plate were transduced with 50 μl of fresh, 45 μm-filtered virus-containing supernatant. The plates were cultured for 5 days before co-culturing with target cells.

To evaluate the functional ability of BCMA CARs to activate JNL cells, they were co-cultured with target cancer cells at different effector to target cell ratios (E:T ratio) to read out their activation by quantifying luciferase expression. The scFv-based CARs R1B6, R1F2, R1G5, PI61, B61-10, B61-02, Hy03, and Hy52 were assessed. The CD19 JNL CAR cells were used as a target specific control, and media alone without target cells served as a negative control.

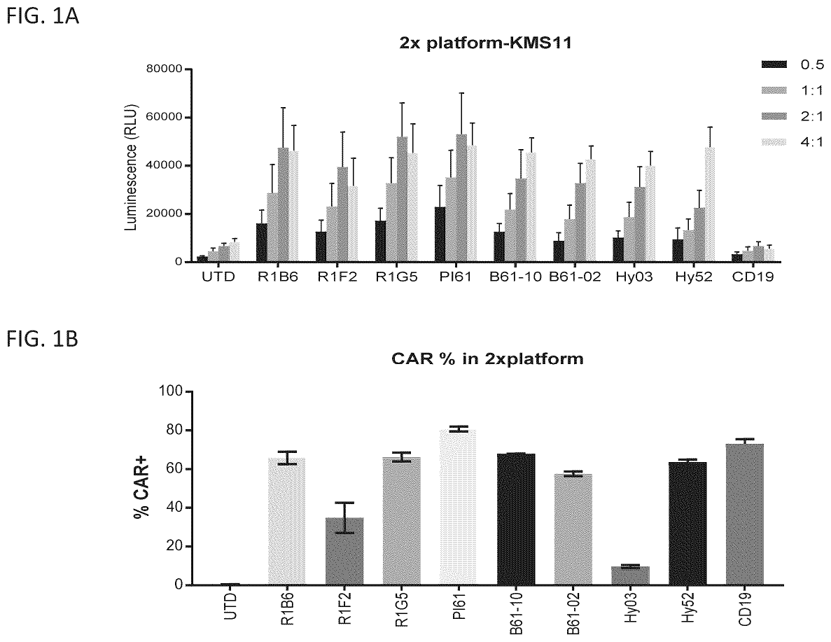

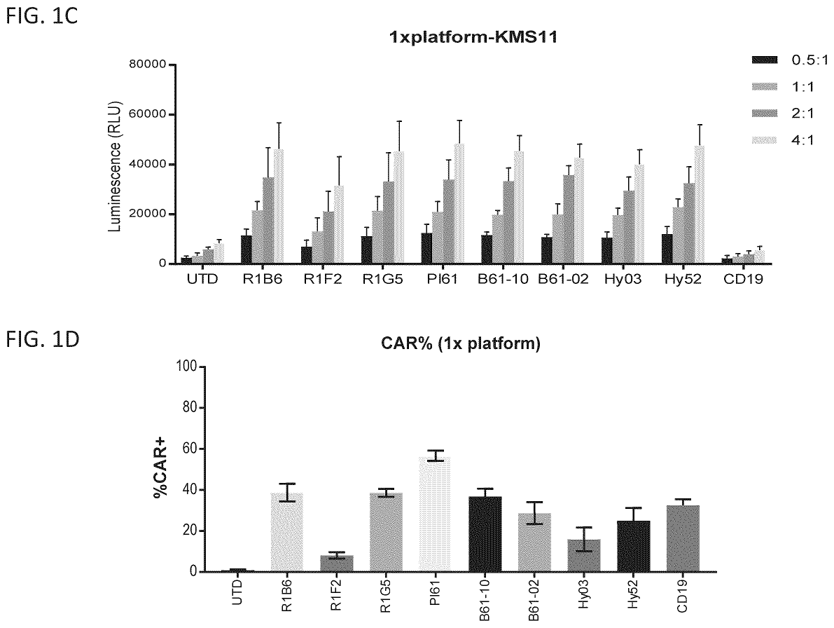

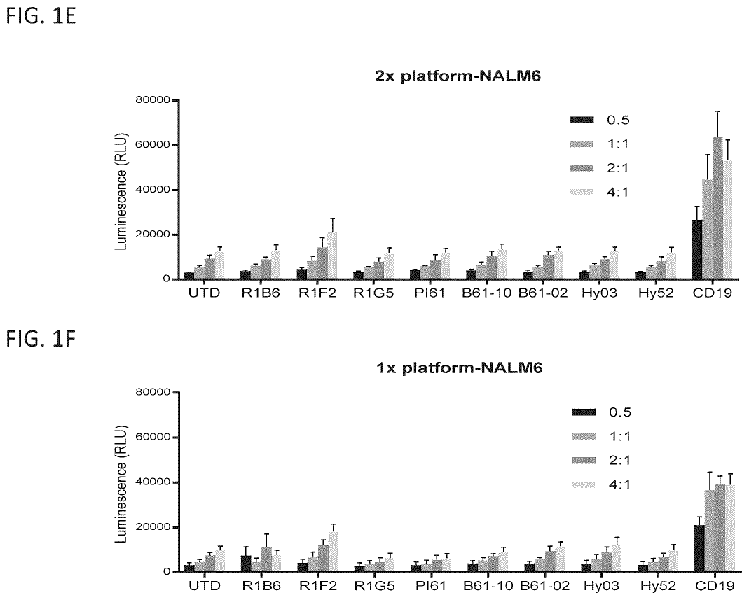

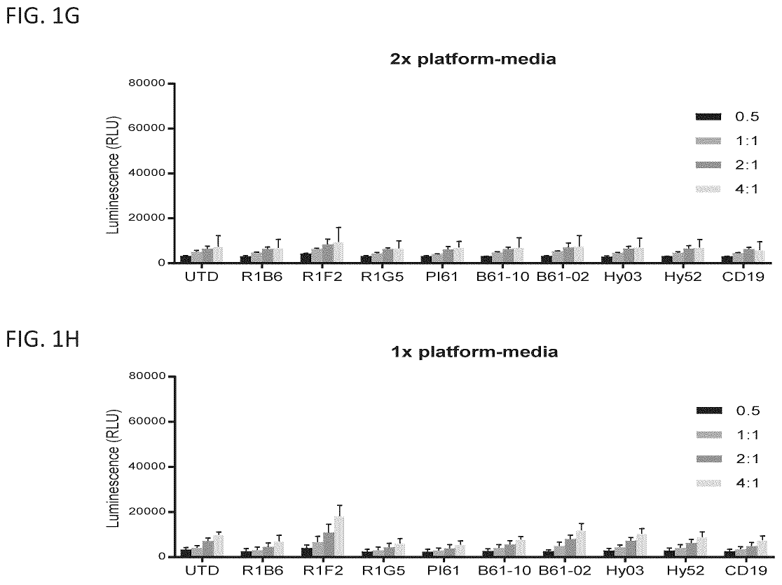

The above mentioned five-day transduced JNL CAR cells were co-cultured with the BCMA-positive multiple myeloma (MM) cell line KMS11, or NALM6, an acute lymphocytic leukemia cell line, served as a BCMA-negative control. Remaining JNL CAR T cells were evaluated for BCMA CAR expression by flow cytometry. Co-cultures were set up in 384-well plates at effector-to-target (E:T) ratios of 4:1, 2:1, 1:1 and 0.5:1 and incubated for 24 h, after which the expression of luciferase by the activated JNL CAR T cells was quantified by Bright-Glo™ Luciferase Assay System (Promega, Madison, WI). The amount of light emitted from each well (luminescence) was a direct read-out of JNL activation by the respective CAR. JNL cells were considered to be activated when the level of luminescence was equal or more than twofold of UTD cells. The BCMA+ KMS11 cell line led to activation of the JNL cells expressing R1B6, R1F2, R1G5, PI61, B61-10, B61-02, Hy03, and Hy52 (FIGS. 1A and 1C). None of the BCMA CARs showed activation by the BCMA-negative line NALM6 (FIGS. 1E and 1F). Media alone, without target cells, did not activate any of the CAR transduced JNLs tested (FIGS. 1G and 1H). FACS analyses demonstrated that BCMA-CAR expression in transduced JNLs was detected to different degrees; CAR % is generally positively correlated with JNL activation by KMS11 cells in the most active JNL CARTs (FIGS. 1B and 1D).

### Generation of BCMA CAR T Cells

The following 8 CARs were chosen for analysis of CAR expression, stability and efficacy in primary T cells: R1B6, R1F2, R1G5, PI61, B61-10, B61-02, Hy03, and Hy52. BCMA CAR T cells were generated by starting with blood from healthy apheresed donors whose T cells (CD4+ and CD8+ lymphocytes) were obtained by negative selection for CD3+ T cells. These cells were activated by the addition of CD3/CD28 beads (Dynabeads® Human T-Expander CD3/CD28, Thermo Fisher Scientific) at a ratio of 1:3 (T cell to bead) in T cell medium (RPMI1640, 10% heat-inactivated fetal calf serum (FCS), 2 mM L-glutamine, 1× Penicillin/Streptomycin, 100 M non-essential amino acids, 1 mM Sodium Pyruvate, 10 mM Hepes, and 55 M 2-mercaptoethanol). T cells were cultured at 0.5×10⁶ T cells in 1 mL medium per well of a 24-well plate at 37° C., 5% CO₂. After 24 hours, when T cells were blasting, T cells were transduced with BCMA CAR virus at a multiplicity of infection (MOI) of 5. T cells began to divide in a logarithmic growth pattern, which was monitored by measuring the cell counts per mL, and T cells were diluted in fresh medium every two days and de-beaded and harvested for further analyses at day 9. Aliquots of T cells were stained to measure CAR expression by flow cytometry at day 5 and 9 on a FACS Fortessa (BD). All BCMA CAR T cells were produced under research grade (i.e., not clinical grade) manufacturing conditions.

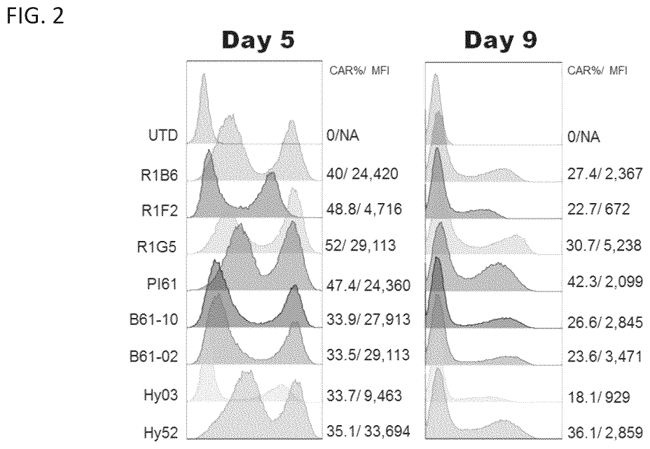

The BCMA-CAR surface expression and its stability was assessed by measuring CAR % and MFI (mean fluorescence intensity) at day 5 and day 9 using flow cytometry analyses of rBCMA_Fc-AF647 stained cells (FIG. 2 and Table 27). BCMA CAR expression in the final product at day 9 differs from construct to construct, ranging from 18% to 42.4%, and MFI from 672 to 5238. Constructs from PALLAS-derived clones R1F2, R1B6, and R1G5, and the hybridoma clone, Hy03 showed ˜30% to 50% CAR loss from day 5 to day 9, while P161, B61-10 and -02, as well as Hy52 are relatively stable in terms of the percentage of CAR expression, though all the CAR constructs showed a decrease in MFI from day 5 to day 9, which was probably due to the smaller size of T cells at their resting stage on day 9. The cell counts of the CAR T cell cultures indicated that there is no detectable negative effect of the human scFv bearing BCMA CAR on the ability of the cells to expand normally when compared to the untransduced T cells (“UTD”).

TABLE 27
Analysis of CAR expression

| CAR | | CAR % on T cells | | CAR MFI on T cells | |
|---|---|---|---|---|---|
| Construct | Titer | Day 5 | Day 9 | Day 5 | Day 9 |
| R1B6 | 2.68E+08 | 40.0 | 27.4 | 24,420 | 2,367 |
| R1F2 | 3.60E+08 | 48.8 | 22.7 | 4,716 | 672 |
| R1G5 | 2.27E+08 | 52.0 | 30.7 | 29,113 | 5,238 |
| PI61 | 1.71E+08 | 47.4 | 42.3 | 24,360 | 2,099 |
| B61-10 | 7.06E+07 | 41.1 | 30.3 | 27,298 | 3,288 |
| B61-02 | 8.16E+07 | 33.5 | 23.6 | 29,113 | 3,471 |
| Hy03 | 4.96E+07 | 33.7 | 18.1 | 9,463 | 929 |
| Hy52 | 7.03E+07 | 35.1 | 36.1 | 33,694 | 2,859 |

### Evaluating Functionality of BCMA CAR-Redirected T Cells

To evaluate the functional abilities of BCMA CAR-T cells, co-cultures were set up with BCMA-positive and -negative cancer lines. CAR-T cells were thawed, counted and co-cultured with target cells to read out their killing capabilities and secretion of cytokines. BCMA CAR-clones R1B6, R1F2, R1G5, B61-02, B61-10, P161, Hy03, and Hy52 were tested. Non-transduced T cells (UTD) were used as non-targeting T cell controls.

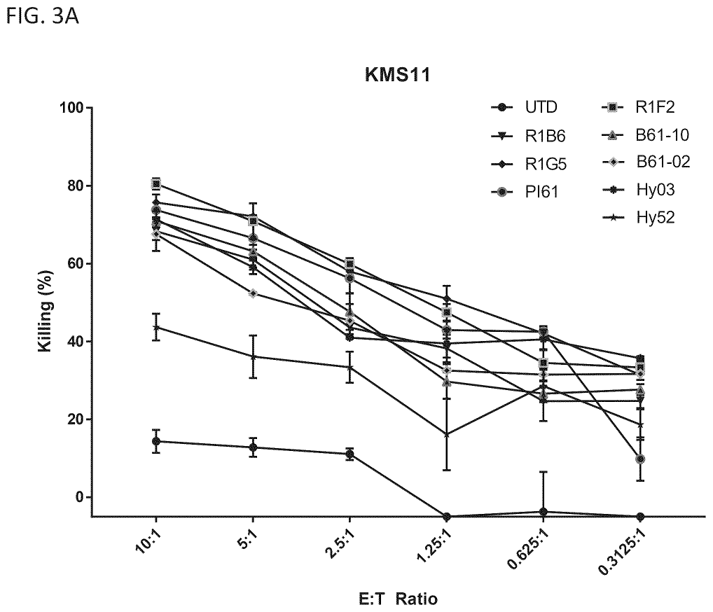

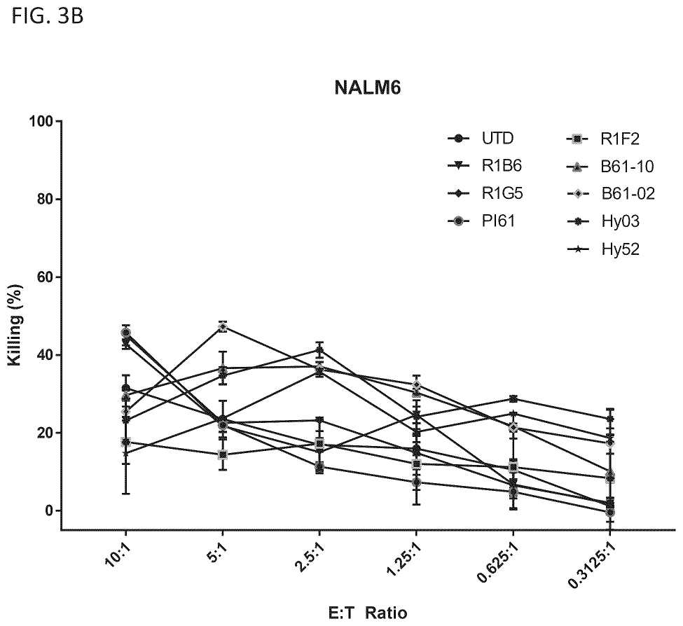

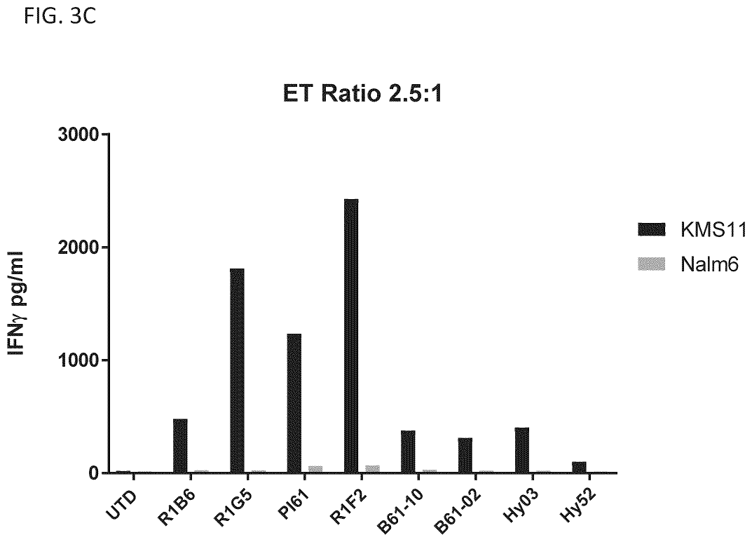

CART cell killing was performed by co-culturing CART cells with KMS11-Luc and NALM6-Luc target cells at different E:T ratios for 20 hours. CAR T cell populations were normalized to equivalent percentages of CAR-positive cells before plating. The cytokine IFNγ was measured in supernatants from 20 hour co-cultures of CAR-T cells with target cells at effector to target ratio of 2.5:1 using the Meso Scale Discovery (MSD; Gaithersburg, MD) and the results for each cytokine were calculated in pg/ml using known standards. All assays were performed in duplicate from a single source of donor cells. Killing data shows that all the BCMA CAR clones kill KMS11 cancer cells effectively (FIG. 3A). The control target cell NALM6 was not killed by any of these BCMA-specific CARs (FIG. 3B). The ability of these CARs to produce IFN-γ when cultured with KMS11 was also tested (FIG. 3C). BCMA CAR R1F2, R1G5 and P161 led to the highest amounts of IFN-γ being produced. Levels of cytokine produced by BCMA CARTs after exposure to the control NALM6 cells were low (FIG. 3C), indicating no unspecific activation by BCMA CARs.

### Conclusions

New BCMA-binding scFvs were tested in the context of CAR T cells. Eight CARs were assayed in a JNL reporter assay as well as in primary T cells: R1B6, R1F2, R1G5, B61-02, B61-10, P161, Hy03, and Hy52. All eight CAR-T cells showed target-specific killing. T cells expressing R1F2, R1G5, or PI61 produced the highest amounts of IFN-γ in the presence of target cells. Overall, the transfer of BCMA CARs to primary T cells induced anti-BCMA CAR reactivity but no off-target function.

### Example 2: Dual CAR Expression and In Vitro Activity of Anti-BCMA and Anti-CD19 Dual CARTs

A set of bicistronic constructs comprising two full CAR (chimeric antigen receptor) chains, one directed to BCMA and the other to CD19, was engineered in a lentiviral vector (Table 28). CAR expression is driven by the EF1alpha promoter. Such CARs comprise a set of human single chain variable fragments (scFv) targeting BCMA (duBCMA.4, P161, R1G5, and R1B6). The same humanized scFv targeting CD19 was engineered in all the constructs. At the N-terminus of each scFv, a signal peptide derived from CD8 alpha targets the CAR to the secretory pathway. Such a signal peptide is expected to be cleaved co-translationally and therefore be absent in the mature form of the CAR displayed at the cell surface. At the C-terminus of each scFv is the hinge and transmembrane domain of CD8 alpha, fused to the intracellular domain of 4-1BB, followed by the intracellular domain of CD3zeta. Between the two CARs, more precisely between the last amino acid of the first CD3zeta domain and the signal peptide of the subsequent CAR, is engineered a linker (GSG (SEQ ID NO: 206)) followed by 2A self-cleaving peptide from porcine teschovirus-1 2A (i.e., P2A sequence). Other linkers and/or self-cleaving peptides could be used as well. This design affords expression of two independent CARs, from a single mRNA transcript. The DNA sequences encoding the overlapping regions between the two CARs (signal peptide, hinge, transmembrane domain, 4-1BB, and CD3zeta) are distinct from one another in order to minimize potential recombination.

TABLE 28
Summary of constructs.

| Construct NO. | Description |
|---|---|
| 234 | duCD19.1-duBCMA.4 |
| 235 | duBCMA.4-duCD19.1 |
| 236 | R1G5-duCD19.1 |
| 237 | R1B6-duCD19.1 |
| 238 | PI61-duCD19.1 |
| 244 | Mono-duCD19.1 |
| 245 | Mono-duBCMA.4 |

The constructs were used to make vector material, which was used to infect human primary T cells. CAR expression was assessed by flow cytometry. The quantity and quality of effector T cell responses (“BCMA-CD19 dual CART” or “T cells”) in response to BCMA expressing (“BCMA+” or “BCMA positive”) and CD19+ tumor targets were also measured. Effector T cell responses include, but are not limited to, cellular expansion, proliferation, doubling, cytokine production and target cell killing or cytolytic activity (degranulation).

### Lentivirus Production and Titer Determination

The five constructs, encoding the dual BCMA/CD19 CARs, described above were used to produce genomic material packaged into VSVg pseudotyped lentiviral particles. Two constructs were used as controls: one encoding a mono CAR directed against BCMA (duBCMA.4) and another encoding a mono CAR directed against CD19. All seven constructs were engineered in the same plasmid backbone. Each of these DNAs was mixed with the three packaging components VSVg, gag/pol and rev in combination with lipofectamine reagent to transfect Lenti-X 293 T cells (Clontech), followed by medium replacement 12-18 h later. 30 hours after medium change, the media was collected, filtered and stored at −80° C.

Lentiviral titer was determined by evaluating the surface expression of BCMA-CD19 dual CARs on transduced Sup-T1 cells using recombinant human Alexa-647-Fc-tagged BCMA protein (BCMA-Fc) and antilD-duCD19.1 antibody (PE). Sup-T1 cells were transduced with a 3-fold serial dilution of viral supernatants with a starting dilution of 1:3. The percentage of cells expressing the CAR (CAR+ cells) was assessed four days later. Viral titer was calculated either using the upstream CAR positivity or dual positive CAR population according to the following formula:

(% CAR+)×(# Sup-T1 cells seeded)×(Dilution)/(Amount of Virus (mL))

Viral titer was calculated from the central most dilution point in the linear range giving between 5 and 25% CAR+ cells (Table 29).

TABLE 29
supTI titers.

| Construct | Based on CAR+ of | Based on the double+ |
|---|---|---|
| NO. | the upstream CAR (a) | CAR (b) |
| 234 | 5.39E+07 | 1.91E+07 |
| 235 | 4.41E+08 | 1.15E+08 |
| 236 | 2.14E+08 | 1.17E+08 |
| 237 | 1.97E+08 | 8.51E+07 |
| 238 | 1.85E+08 | 5.66E+07 |

### Generation of BCMA-CD19 CAR T Cells Using Conventional 10-Day Production Process

BCMA-CD19 dual or monoBCMA or monoCD19 CART cells were generated using human primary T cells (CD4+ and CD8+ lymphocytes) obtained by negative selection or positive selection via Prodigy upon processing blood from healthy apheresed donors. Before transduction, the T cells were activated using CD3/CD28 beads (Dynabeads® Human T-Expander CD3/CD28, Thermo Fisher Scientific) at a ratio of 1:3 (T cell to bead) in T cell medium (RPMI1640, 10% heat-inactivated fetal calf serum (FCS), 2 mM L-glutamine, 1× Penicillin/Streptomycin, 100 M non-essential amino acids, 1 mM Sodium Pyruvate, 10 mM Hepes, and 55 M 2-mercaptoethanol). The cells were cultured at a density of 0.5×10⁶/mL medium per well in a 24-well plate at 37° C., 5% CO₂. After 24 hours, when T cells were blasting, they were transduced with viruses at a multiplicity of infection (MOI) of 5 based on the upstream CAR titer. T cells began to divide in a logarithmic growth pattern, which was monitored by measuring the cell counts per mL, and T cells were diluted in fresh medium every two days and de-beaded and harvested for further analyses on day 8 or beyond depending on the size of the cells. Aliquots of T cells were stained to measure CAR expression by flow cytometry on day 7 or 8 on a FACS Fortessa (BD).

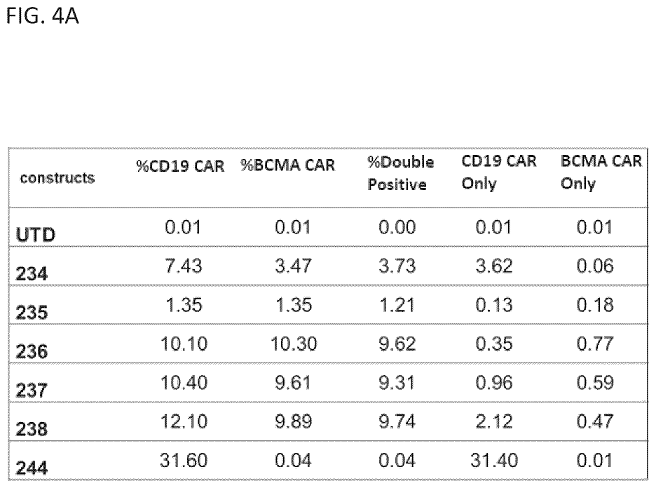

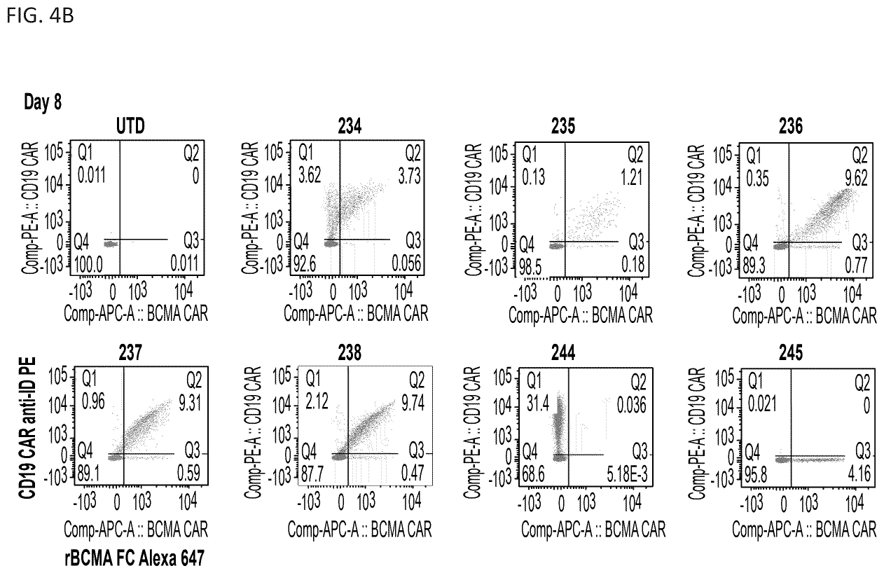

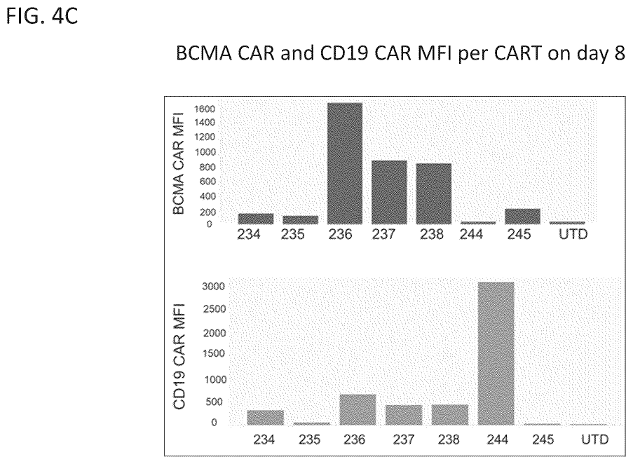

The BCMA-CAR and CD19-CAR surface expression was assessed by measuring CAR % and MFI (mean fluorescence intensity) at day 8 using flow cytometry analyses of rBCMA_Fc-AF647 and antiID-duCD19.1 (PE) stained cells (FIGS. 4A-4C). The three dual CAR vectors comprising R1G5, R1B6, or PI61 led to a similar percentage of double positive CARs (˜10%), with construct 236 (R1G5/duCD19.1) displaying the highest MFI levels for BMCA and CD19 CARs (FIG. 4A). The mono duCD19.1 (construct 244) displays the highest MFI for CAR19 across the different constructs (FIG. 4B).

### Evaluating Functionality of Dual BCMA-CD19 CAR-Redirected D8 T Cell Product In Vitro and In Vivo

To evaluate the anti-tumor efficacy of each of the CARs within the dual BCMA-CD19 CART cells, co-cultures were set up with BCMA positive (KMS11), CD19 positive (NALM6) and BCMA/CD19 negative cancer lines (CD19KO; NALM6-derived). CART cells were thawed, counted and co-cultured with target cells to read out their killing capabilities and secretion of cytokine. Non-transduced T cells (UTD) were used as non-targeting T cell controls.

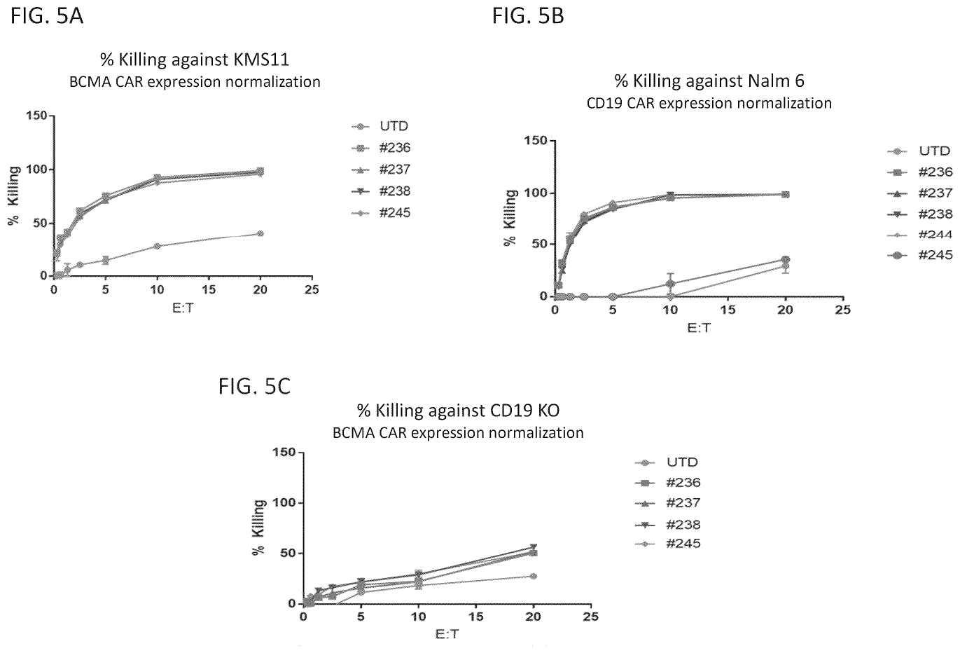

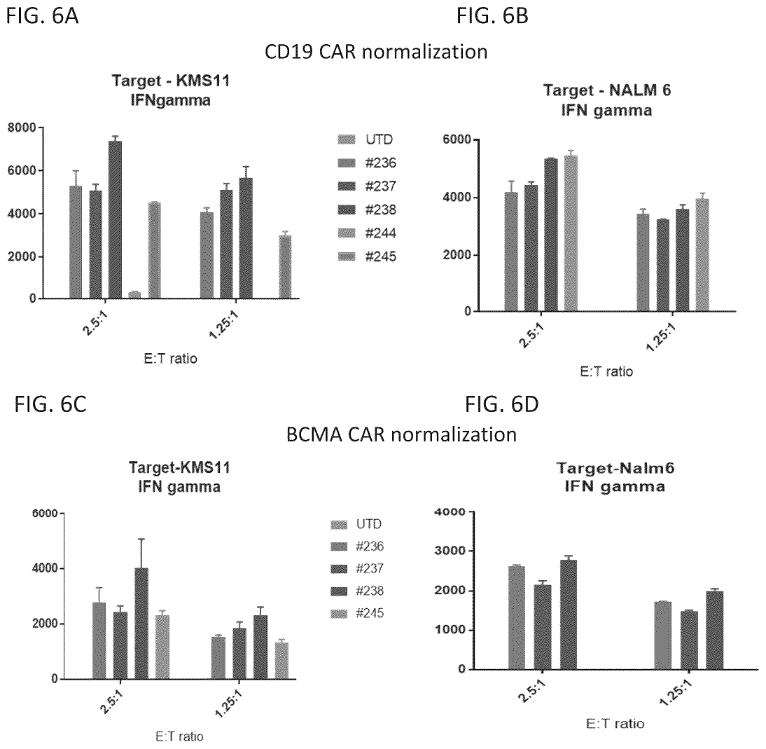

The in vitro cytotoxic assay was performed by co-culturing CART cells with target cells expressing luciferase at different E:T ratios for 20 hours. CART cell populations were normalized to equivalent percentages of CAR-positive cells before plating, according to the CAR directed to the respective tumor (i.e., normalization based on BCMA CAR when CARTs were co-cultured with KMS11 cells; and normalization based on CAR19 when CARTs were co-cultured with NALM6). The cytokine IFNγ was measured in the supernatants of the 20-hour co-cultures, corresponding to a target ratio of 1.25:1 or 2.5:1 (CAR-T cells:target cells), using the Meso Scale Discovery (MSD; Gaithersburg, MD). The results were calculated in pg/mL using known standards. All assays were performed in duplicate from a single source of donor cells. Killing data show that all dual CAR clones were effective against BCMA-positive KMS11 cells and CD19-positive Nalm6 cells (FIGS. 5A and 5B, respectively). Only background killing was observed against the BCMA/CD19 negative tumor cells (FIG. 5C). The ability of these dual CARs to produce IFN-γ when co-cultured with KMS11 or Nalm6 was similar as assessed using two different E:T ratios (FIGS. 6A-6D).

The in vivo anti-tumor activity of these D8 CARTs is analyzed using mixed xenograft tumor model (5% Nalm6 and 95% KMS11) in NSG mice.

Production and Measurement of Day 1 Anti BCMA-CD19 Dual CARTs with Activated Rapid Manufacturing (ARM) Process

In some embodiments, this ARM process starts with a frozen or fresh leukapheresis product. After a sample for counting and QC is obtained, the product is attached to a cell sorting machine (e.g., an installed CliniMACS Prodigy device kit) and the program begins. The cells are washed and incubated with microbeads that bind to desired surface markers, such as CD4 and CD8. The bead-labeled cells are selected by passing the cells through a magnetic column. Isolated cells are washed again and the separation buffer is exchanged for cell media. Purified T cells then either proceed to culture or are cryopreserved for later use. Purity of the isolated T cells will pass a QC step by flow cytometry assessment. Cryopreserved cells are thawed, washed in pre-warmed cell media, and resuspended in cell media. Fresh cells are added to culture directly. The cells are seeded into membrane bioreactors at 0.4-1.2 e6 cells/cm² of membrane, an activating reagent such as anti-CD3/anti-CD28 beads/polymers, nanoparticles, or nanocolloids is added, and cell media is added to a final volume of 0.25-2 ml/cm² of membrane. For in vitro CAR expression kinetics study, a 24 well Grex is used. A Grex100, flask, or centricult is used to test whether this manufacturing process is scalable and to test in vivo anti-tumor efficacy. At the time of plating, the cells are transduced with a lentiviral vector encoding BCMA-CD19 dual CAR at a multiplicity of infection (MOI) of 1 or 2. MOI is determined based on the viral titer obtained in SupT1 cells, basing on the titer of the CAR that is engineered upstream of the 2A sequence. At 24 hours, the cells are washed to remove unnecessary reagents before staining to measure the CAR expression by flow cytometry and reformulated in cryopreservation media as “day 1 CART product” for in vivo study. In all cases, an aliquot of cells is harvested at 72 h post transduction for measuring CAR expression kinetics in vitro. The day 1 CART responses include, but are not limited to, in vivo cytolytic activity and expansion.

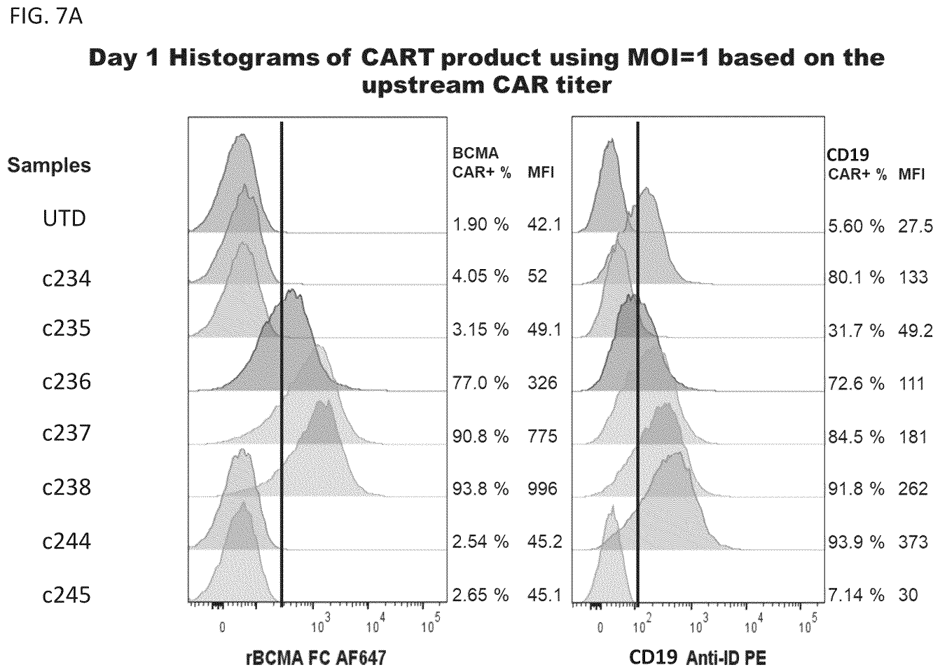

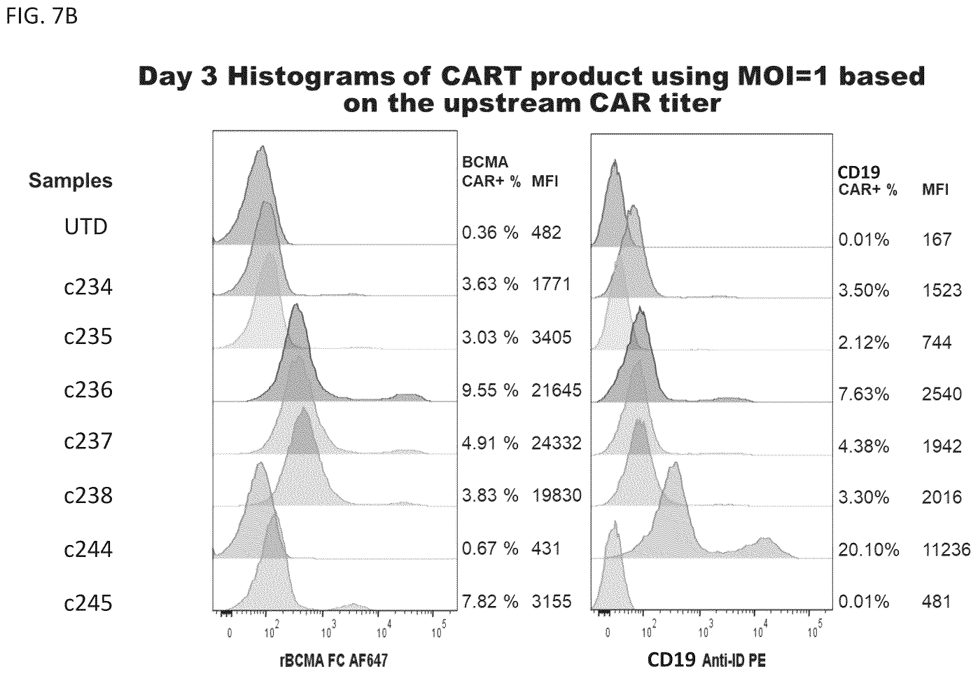

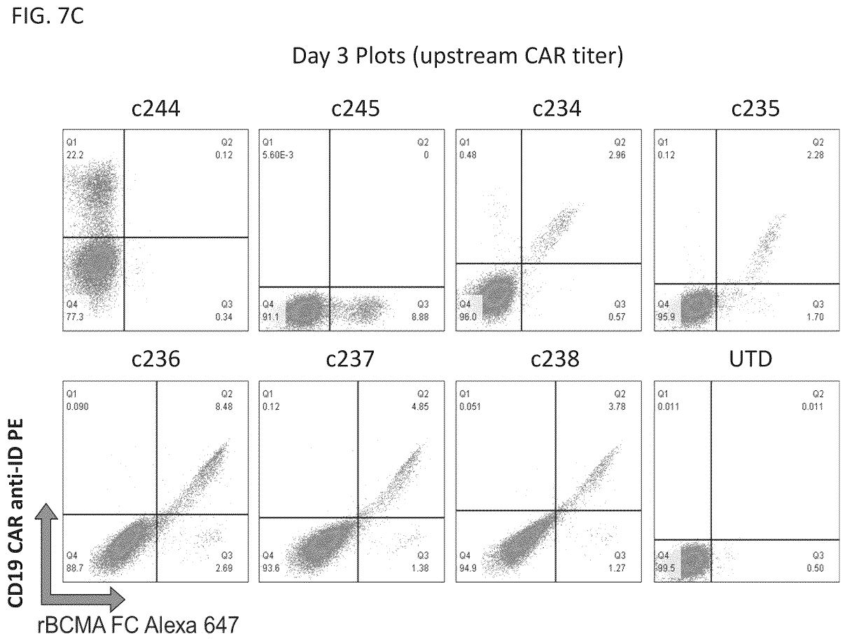

FIGS. 7A-7B show the expression pattern of both anti-BCMA and anti-CD19 CARs, at 24 h and 72 h post-transduction of human primary T cells manufactured using the ARM process, using a MOI of 1 based on the SupT1 titer determined by expression of the upstream CAR only. Twenty-four hours post-transduction, it was observed in a flow cytometry analysis that the whole population of live CD3+ T cells shifted to the right at different degrees (FIG. 7A). This expression pattern was different from a typical flow cytometry histogram of cells transduced to express a CAR, where a CAR positive population is clearly separated from a negative population. These data suggest “pseudotransduction or transient expression” may be detected by the rBCMA_Fc flow cytometry staining reagent. It has been previously reported that lentiviral pseudotransduction was observed from the time of vector addition to 24 hours in CD34+ cells and up to 72 hours in 293 cells (Haas D L, et al. Mol Ther. 2000. 291: 71-80). Integrase-defective lentiviral vector caused transient eGFP expression for up to 10 days in CD34+ cells and for up to 14 days in 293 cells. Although the observed CAR expression on day 1 post T cell transduction may be potentially attributed to pseudotransduction, on day 3, however, two clear populations emerged, one that was rBCMA_Fc positive and the other anti-ID duCD19.1 positive (FIG. 7B and FIG. 7C). Furthermore, 20 to 30% of cells were monoBCMACAR positive (CD19CAR negative) in cells engineered using the dual constructs c235, c236, c237, and c238 (FIG. 7C). Altogether, these results demonstrate that both CARs were well expressed in the dual CART system when RIG5, R1B6, and P161 were used in combination with duCD19.1. 72 h post viral transduction could serve as a surrogate time point for measuring CAR expression for in vivo dosing strategy.

### Evaluating Functionality of the Day 1 ARM Processed BCMA CART In Vivo

The day 1 CARTs generated using the centricult or flask system were examined for their anti-tumor activity in vivo using three mouse models: a disseminated KMS-11-luc (BCMA+CD19−) multiple myeloma model, a Nalm6-Luc (CD19+BCMA−) xenograft mouse model, and a mixed model of 95% KMS-luc with 5% NALM6-Luc cells. The aims of this in vivo study was threefold: (1) demonstrating efficacy of both the BCMA and CD19 arms of the dual CARTs; (2) comparing the mixed model (comprising BCMA+ tumor cells and CD19+ tumor cells) to the KMS-11-luc multiple myeloma model (BCMA+CD19−) to understand potential activation of the dual CARTs through the CD19 target; and (3) testing the dual CARTs in the Nalm6 alone model (CD19+BCMA−) to examine the activity of the CD19 arm. BLI measurements were taken twice weekly. Peripheral blood was taken at days 6, 13, 20 and 27 for flow cytometry analysis. Plasma was collected on previously mentioned days along with day 2 for cytokine analysis. The study design and dose information are summarized in Table 30.

TABLE 30
In vivo study design and dose regime

| | | | Both Luc tagged |
|---|---|---|---|
| | KMS11 | Nalm6 | KMS11 (BCMA) and |
| | (BCMA) | (CD19) | Nalm6 (CD19) mix |
| c236 | 1e4, 5e4 | 1.5e5 | 1e4, 5e4 |
| c238 | 1e4, 5e4 | 1.5e5 | 1e4, 5e4 |
| Mono PI61 | 1e4, 5e4 | | 1e4, 5e4 |
| Mono CTL119 | | 1.5e5 | 1e4, 5e4 |

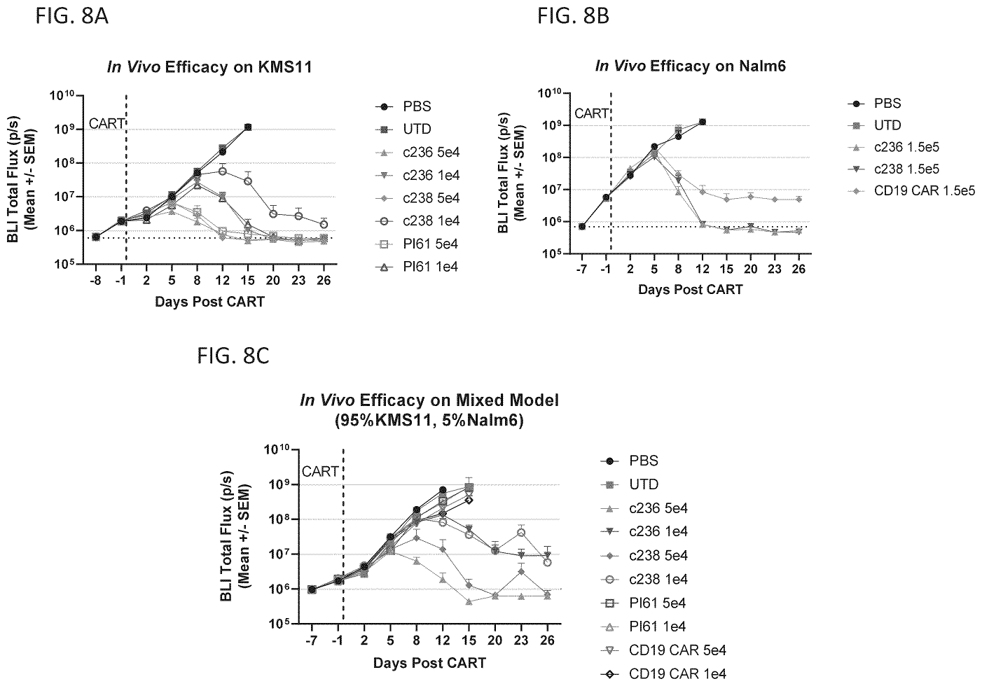

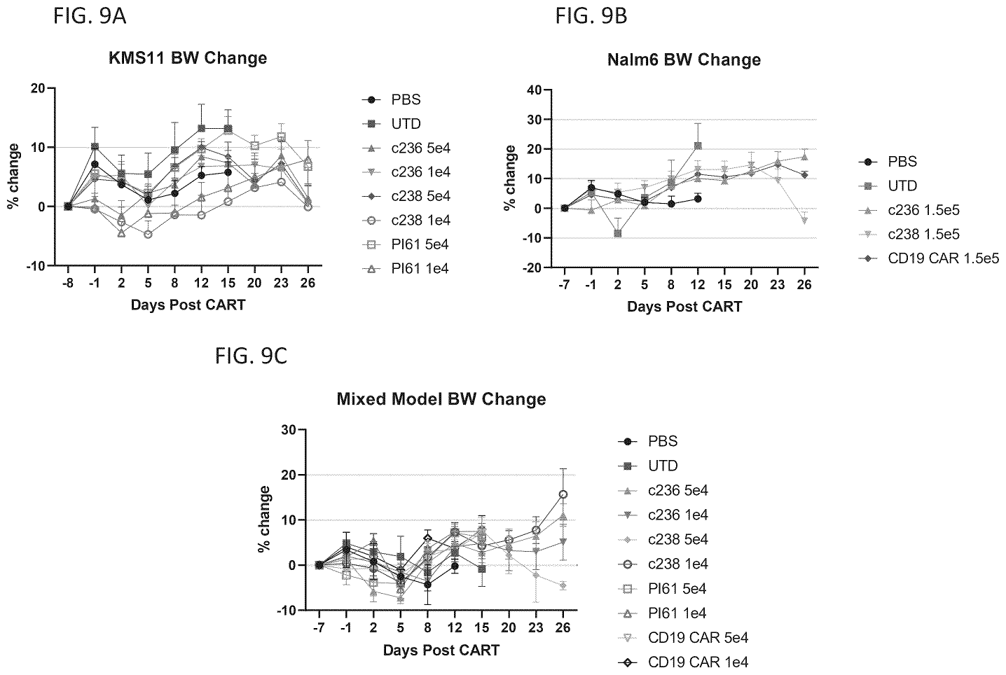

The dual CARTs cleared mixed tumors (BCMA+CD19+) at the dose of 5 e4, and inhibited but not fully eliminated tumors at the dose of 1 e4 over the course of the study (FIG. 8C). Neither P161 nor CTL119 could control the mixed tumor (FIG. 8C). Both duals c236 and c238 exhibited similar or superior efficacy compared to the mono counterparts in the KMS11 and Nalm6 models (FIGS. 8A and 8B). Body weights increased during the study in all three models (FIGS. 9A-9C). There was a slight drop at the end of the KMS11 and NALM6 study possibly due to GVHD.

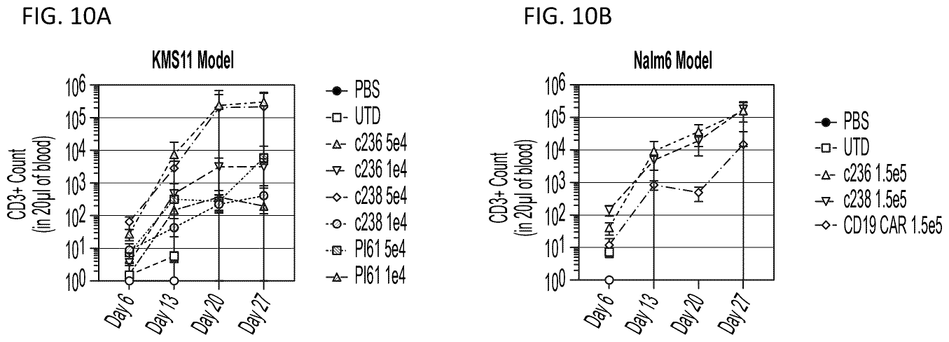

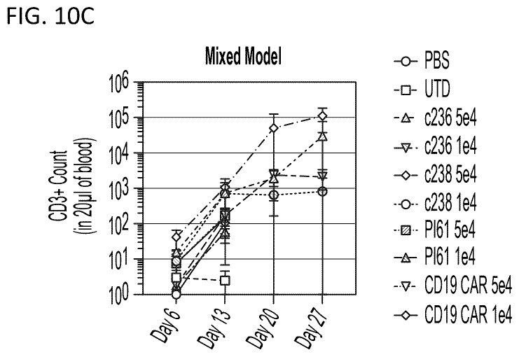

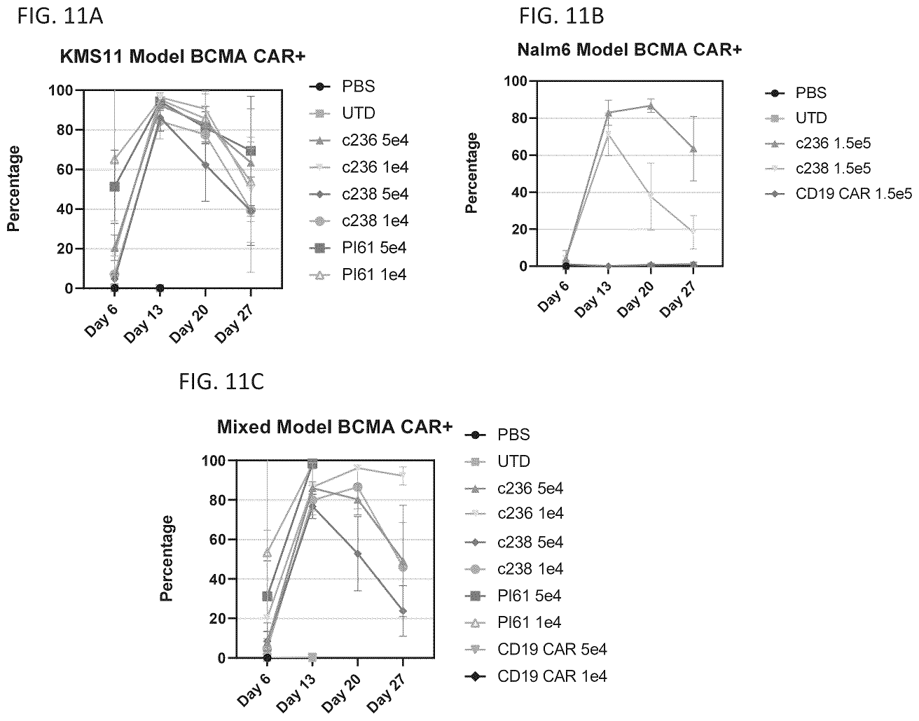

T cell expansion occurred from Day 6 to Day 20, and then evened out from Day 20 to Day 27 (FIGS. 10A-10C). Expansion was dose related across all three models (FIGS. 11A-11C). The KMS11 model showed 3-4 folds higher expansion compared to the mixed model in the higher doses (FIGS. 10A and 10C). Duals c236 and c238 showed higher expansion than mono CARTs (FIGS. 10A-10C).

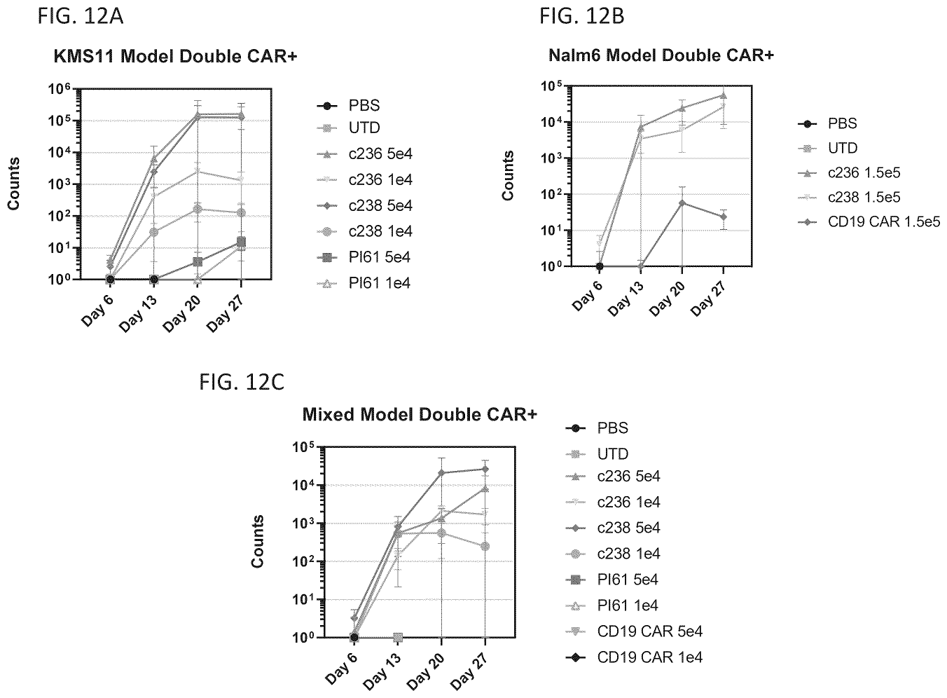

Total BCMA CAR+ percentage peaked at Day 13 and then began to decrease through Day 27 (FIGS. 11A-11C). In the mixed model, the 5 e4 groups showed lower BCMA CAR+ percentages compared to the 1 e4 groups after Day 13 (FIG. 11C). The double CAR+ percentage was related to an influx of total CD3+ cells, which could be a possible sign of GVHD. Double anti-BCMA and CD19 CAR+ T cell expansion was observed in c236 and c238 CART Rx groups in all three models (FIGS. 12A-12C).

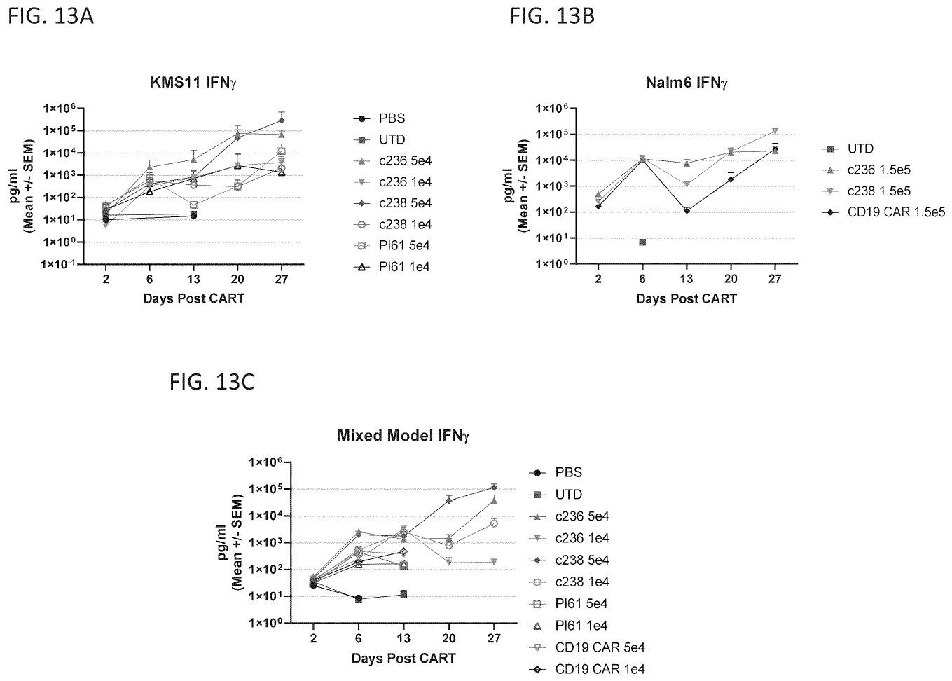

Induction of IFNγ was dose responsive across all the models (FIGS. 13A-13C). Duals at both doses produced ˜3 to 4 folds more IFNγ at peak in the mixed model than that in the KMS11 alone model (FIGS. 13A and 13C). Peak induction was observed within 13 days in most of the groups in all the models (day 20 and day 27 peaks were most likely due to GVHD) (FIGS. 13A-13C).

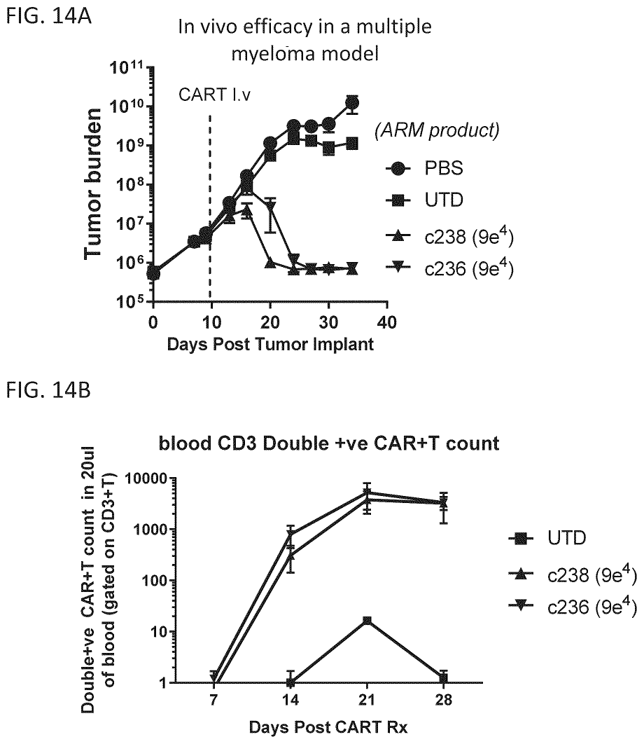

In a separate study, the day 1 CARTs generated using Grex100 and 6-well Grex system were examined for their anti-tumor activity in vivo using a disseminated KMS-11-luc multiple myeloma model. The luciferase reporter allows for monitoring of disease burden by quantitative bioluminescence imaging (BLI). Briefly, day 1 CARTs manufactured as described above were administered in tumor-bearing mice. Blood samples were taken weekly to measure peripheral blood CART expansion and analyzed by flow cytometry. T cells engineered with construct #236 and construct #238 displayed potent anti-tumor activity (FIG. 14A) and good CART expansion (FIG. 14B) in vivo towards a KMS11 (BCMA+CD19−) model.

### Example 3: Characterization of Diabody CARTs

This example describes characterization of diabody CARs JL1 to JL10. JL1 to JL8 are P161/CTL119 diabody constructs and JL9 to JL10 are R1G5/CTL119 diabody constructs. The sequence information of JL1 to JL10 is disclosed in Table 31.

Production and Measurement of Day 1 Anti BCMA-CD19 Diabody CARTs with Activated Rapid Manufacturing (ARM) Process

In some embodiments, this ARM process starts with a frozen or fresh leukapheresis product. After a sample for counting and QC is obtained, the product is attached to a cell sorting machine (e.g., an installed CliniMACS Prodigy device kit) and the program begins. The cells are washed and incubated with microbeads that bind to desired surface markers, such as CD4 and CD8. The bead-labeled cells are selected by passing the cells through a magnetic column. Isolated cells are washed again and the separation buffer is exchanged for cell media. Purified T cells then either proceed to culture or are cryopreserved for later use. Purity of the isolated T cells will pass a QC step by flow cytometry assessment. Cryopreserved cells are thawed, washed in pre-warmed cell media, and resuspended in cell media. Fresh cells are added to culture directly. Aliquots of frozen Pan T isolated cells are thawed in a 37° C. water bath, put into Optimizer CM (Gibco Optimizer Media with Supplement +100 U/mL human IL2) and spun for 5 minutes at 1500 rpm. Cells are counted and plated into a 24-well plate at 3 e6/mL, 1 mL/well. TransAct is added to each well at 1/100 (10 μL/well).

For in vitro CAR expression kinetics study with the ARM process, a 24-well plate was used. At the time of plating, the cells were transduced with a lentiviral vector encoding BCMA-CD19 diabody CAR at a multiplicity of infection (MOI) of 2. MOI was determined based on the viral titer obtained in SupT1 cells based on the double positive CAR expression. After 24 hours in culture, cells were harvested and washed three times in PBS+1% HSA. Cells were then counted and re-plated at 1 e6/mL final in a 24-well plate. 72 hours after re-plating, cells were harvested, counted and an aliquot of 5 e5 cells from each sample was taken for flow cytometry analysis. This procedure was repeated 72 hours later for a day seven time point.

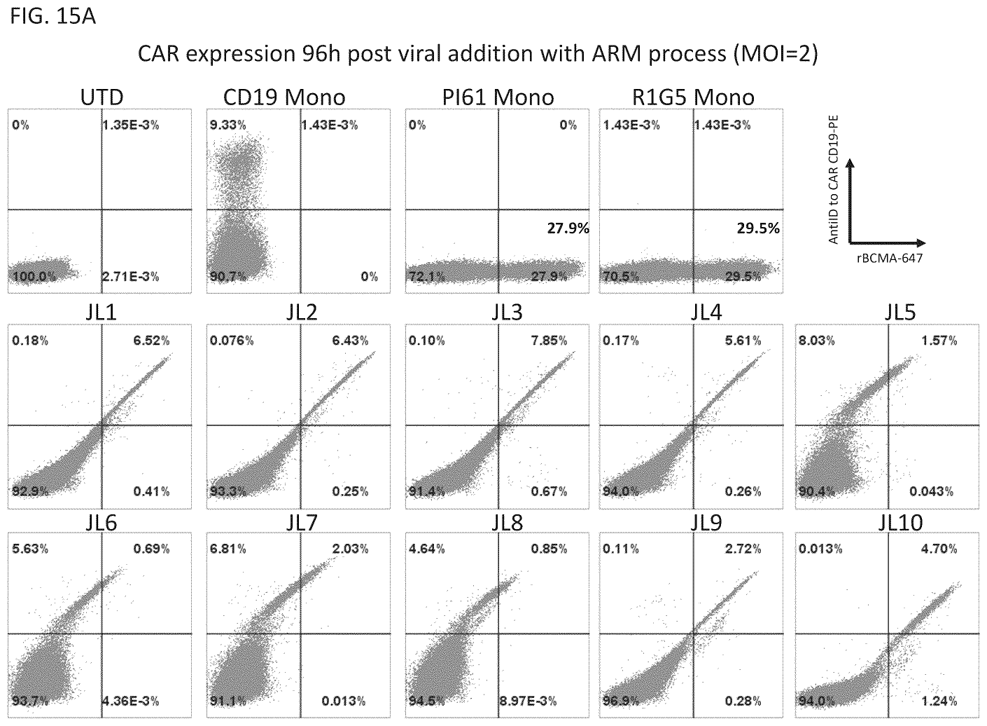

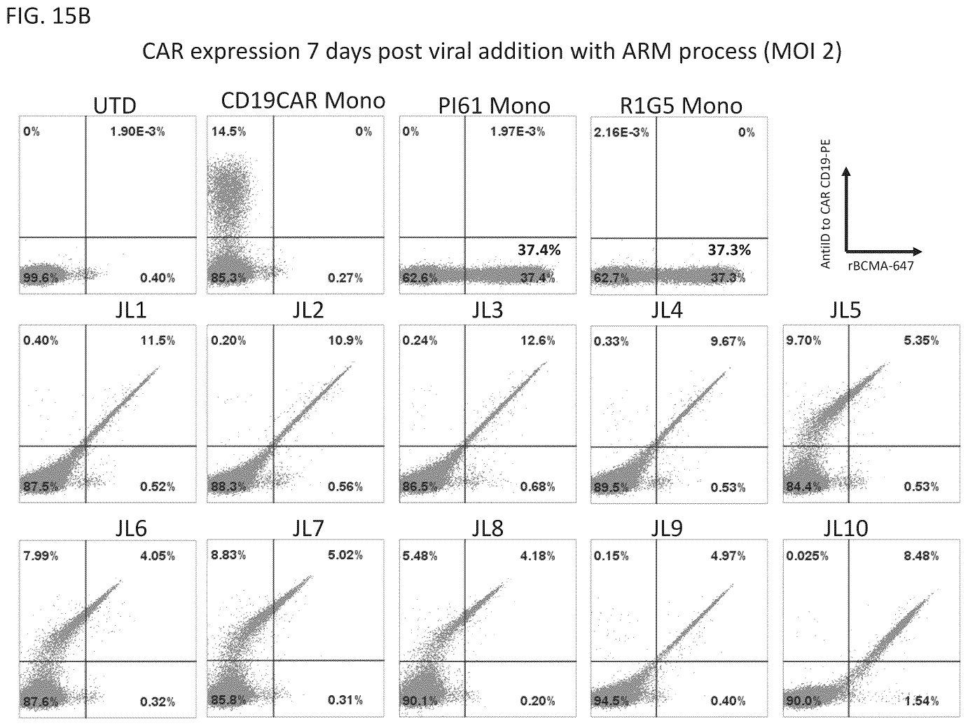

FIGS. 15A and 15B show the expression pattern of both anti-BCMA and anti-CD19 CARs, at 96 h (FIG. 15A) or 7 days (FIG. 15B) post-diabody viral addition to human primary T cells manufactured using the ARM process, using a MOI of 2. Altogether, these results demonstrate that both CARs were well expressed in the diabody CART system when R1G5 or PI61 was used in combination with duCD19.1 on day 4 (FIG. 15A) or day 7 (FIG. 15B) post viral addition. JL1-JL4, JL9, and JL10 showed linear expression of the double positive CAR, while JL5-JL8 showed a slight shift towards CD19+ population, which could be due to CAR binding differences to its respective detecting reagents. Data from one of two donors are shown here.

### CAR-T Cells Production Using Traditional Manufacturing Process

Traditional manufacturing (TM) process is a process where T cells are expanded ex vivo for 8 to 9 days after activation and transduction prior to harvest. Prodigy processed T cells were resuspended in warm RPMI complete T cell medium (RPMI, 10% heat-inactivated FBS, 2 mM L-Glutamine, 100 U/mL Pen/Strep, 1×NEAA, 1 mM Sodium Pyruvate, 10 mM HEPES, and 55 M β-Mercaptoethanol), and plated in 24-well plates at 0.5 e⁶ cells/mL per well. T cells were incubated overnight at 37° C. with Human T-Expander CD3/CD28 beads at a 3:1 ratio of beads-to-cells.

On Day 1, lentiviruses were added at a MOI of 5, based on the SUP-T1titer. No virus was added to the untransduced control (UTD). The T cells were incubated overnight at 37° C. followed by the addition of 1 mL complete T cell medium per well, after which they were incubated overnight at 37° C. For the remaining six days of culture expansion, the T cells were transferred into tissue culture flasks and diluted with complete T cell medium every two days, targeting a concentration of 0.5 e⁶ cells/mL. Typical split ratios ranged from 1:2 to 1:4 during the expansion phase.

On Day 7, the T cells were de-beaded, harvested and cryopreserved in CryoStor CS10 freezing medium, frozen at −80° C. in CoolCell Cell Freezing Containers (Biocision), and transferred to LN₂ the following day. Small aliquots of T cells were stained for CAR expression. Single color controls were included for compensation. Samples were measured on a flow cytometer (BD LSRFortessa), and data were analyzed with FlowJo software.

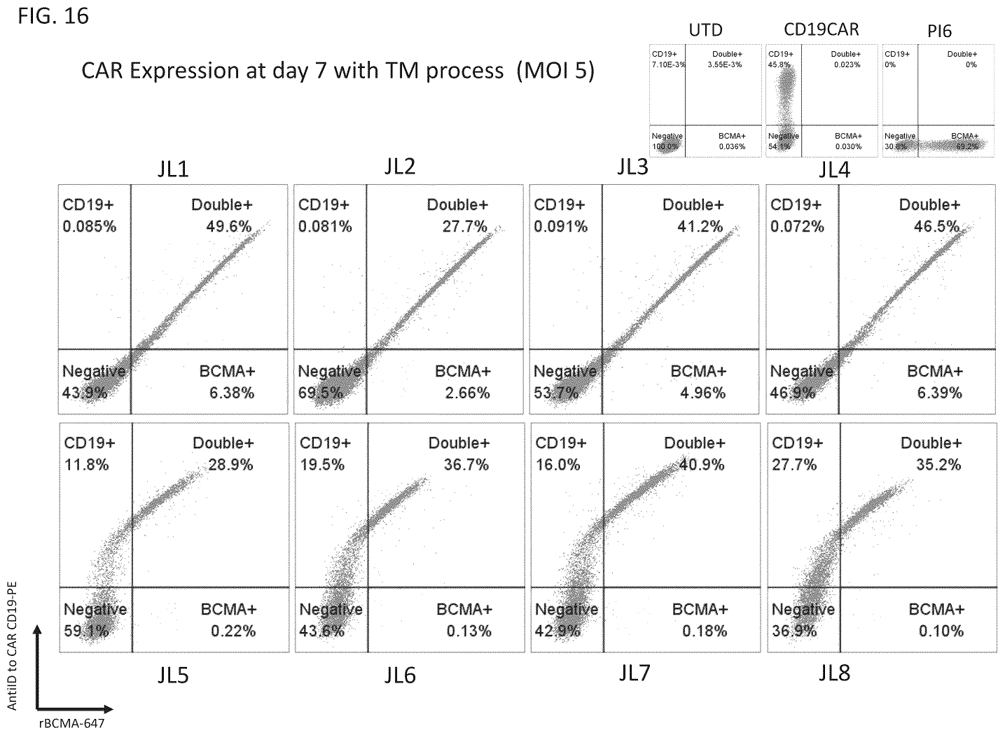

FIG. 16 demonstrates CAR Expression at day 7 with TM process using MOI of 5. The TM products showed a similar expression pattern as the ARM products (FIGS. 15A, 15B, and 16).

### In Vitro Killing Assay

The killing potential of T cells engineered with various diabody constructs in response to BCMA or CD19 expressing target cells was evaluated by incubating CART cells with target cells at 2-fold E:T ratio dilutions starting at 20:1. The number of target cells were fixed at 2.5×10⁵ cells/well and cells were cultured in 96-well flat-bottom plates. Effector cells were CART cells generated using traditional manufacturing by transducing T cells with diabodies. Target cells include BCMA positive KMS11-luc cells or BCMA negative NALM6-luc cells. For this assay, the % transduction of CAR-T cells was normalized by addition of UTD to the BCMA CARTs. This allowed for the comparison of the same number of CARTs and same total T cell number in each sample.

Loss of luciferase signal resulting from cell killing was measured using Bright-Glo substrate 16 h after cell seeding and specific lysis was calculated according to the following formula:

Specific lysis (%)=100−(sample luminescence/average maximal luminescence)*100

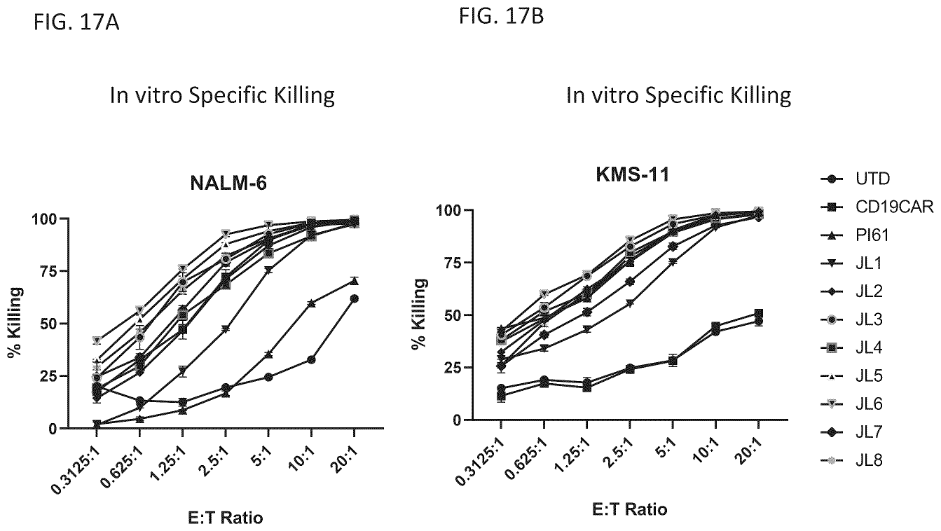

FIGS. 17A and 17B demonstrate the ability of different diabodies of PI61/CTL119 to effectively kill specific target cell lines NALM-6 (CD19+, BCMA−) (FIG. 17A) or KMS-11 (CD19−, BCMA+) (FIG. 17B). The data suggest that clones JL6, JL5, JL3, and JL8 equally killed both target cells.

### Cytokine Secretion Assay

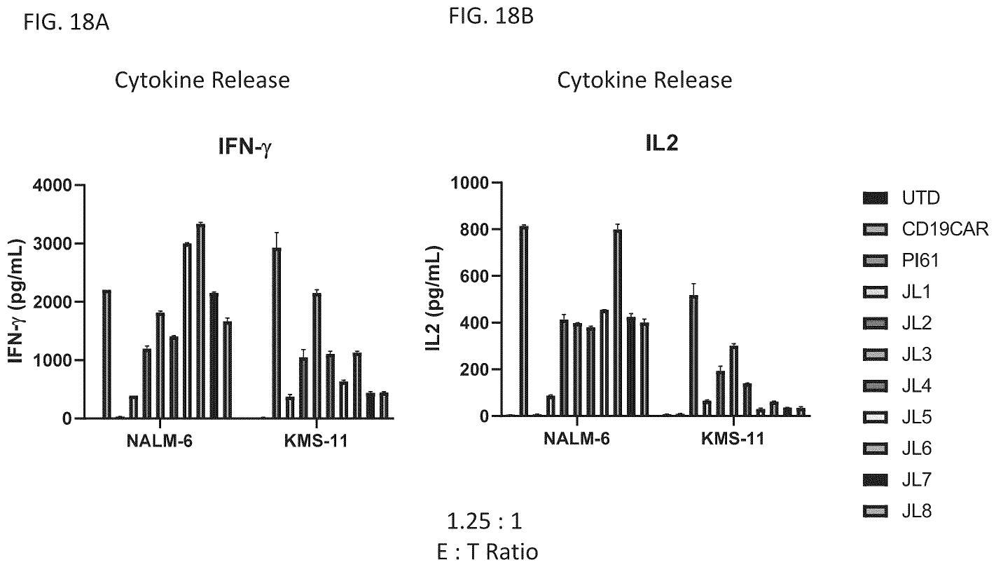

Supernatants were collected from the co-cultures (a ratio of 1.25:1) used in the killing assay above after 20 h incubation to be used in the MSD V-PLEX Human IFN-□□ and IL-2 analysis. Different magnitude of target-specific induction of IFN-□ or IL-2 by diabody transduced cells was observed in response to stimulation with KMS11 or NALM6 cells (FIGS. 18A and 18B). UTD cells did not show any unspecific IFN-□□ secretion in response to either target cells (FIG. 18A). In line with the results from the killing assay, clones JL6, JL5, JL3, and JL8 produced more cytokines in response to the target cells (FIGS. 18A and 18B).

### Example 4: Co-Transduction of BCMA CAR and CD19 CAR

Aliquots of frozen Pan T isolated cells were thawed in a 37° C. water bath, put into Optimizer CM (Gibco Optimizer Media with Supplement+100 U/mL human IL2) and spun for 5 minutes at 1500 rpm. Cells were counted and plated into a 24-well plate at 3 e6/mL, 1 mL/well. TransAct was added to each well at 1/100 (10 μL/well). Virus was added at differing multiplicity of infections (MOIs) based on either the SupT1 titer for PI61 or the qPCR titer for CTL119. PI61 was used at a MOI of 2 or 1 and paired with three different CTL119 MOIs: 1, 0.5, and 0.25. Mono CARs were added at a MOI of 1 or 2 for PI61 and 1 for CTL119, and a UTD control was plated as well. After 24 hours in culture, cells were harvested and washed three times in PBS+1% HSA. Cells were then counted and re-plated at 1 e6/mL final in a 24-well plate.

72 hours after re-plating, cells were harvested, counted and an aliquot of 5 e5 cells from each sample was taken for flow cytometry analysis. Cells were stained with Live/Dead Aqua (BV510) for 15 minutes in 100 μl/well and were then washed twice. The antibody MM (Table 32) was then added at 50 μl/well for 25 minutes at 4° C. Cells were washed twice again and then fixed for 15 minutes in 1.6% PFA in PBS, 100 μl/well. After fixing, cells were washed as previously described and resuspended in a final volume of 150 μl/sample in flow cytometry buffer. 5 e4 cells were acquired on the Live CD3 positive gate of each sample on a BD LSRFortessa (BD Biosciences, San Jose CA) and data was analyzed using FlowJo v. 10 software (Ashland, OR). This procedure was repeated 72 hrs later for a day seven time point.

TABLE 32
Antibody and other reagents.

| Marker | Clone | Fluorochrome | Vendor | Catalogue No. | Dilution |
|---|---|---|---|---|---|
| Live/Dead | | BV510 | Biolegend | 423102 | 1/500 |
| CD3 | SK7 | BUV395 | BD | 564001 | 1/200 |
| CAR19 | Anti-ID | PE | In House Reagent | | 1/160 |
| CD4 | SK3 | PerCP 5.5 | Biolegend | 344608 | 1/100 |
| CAR | rBCMA FC | AF 647 | In House Reagent | | 1/380 (3 ug/ml) |
| CD8 | SK1 | APC H7 | BD | 560179 | 1/200 |

| | | |
|---|---|---|
| FACS Buffer | Miltenyi Biotec | 130-091-222 |
| BSA Stock Solution | Miltenyi Biotec | 130-091-376 |
| Phosphate Buffer Saline (PBS) | Gibco | 14190-144 |
| Para formaldehyde (PFA) | Polysciences Inc. | 18814-10 |

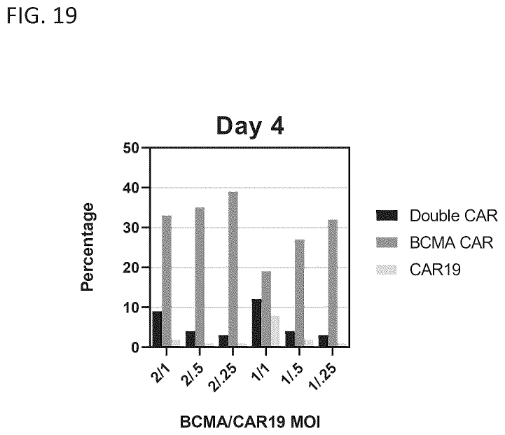

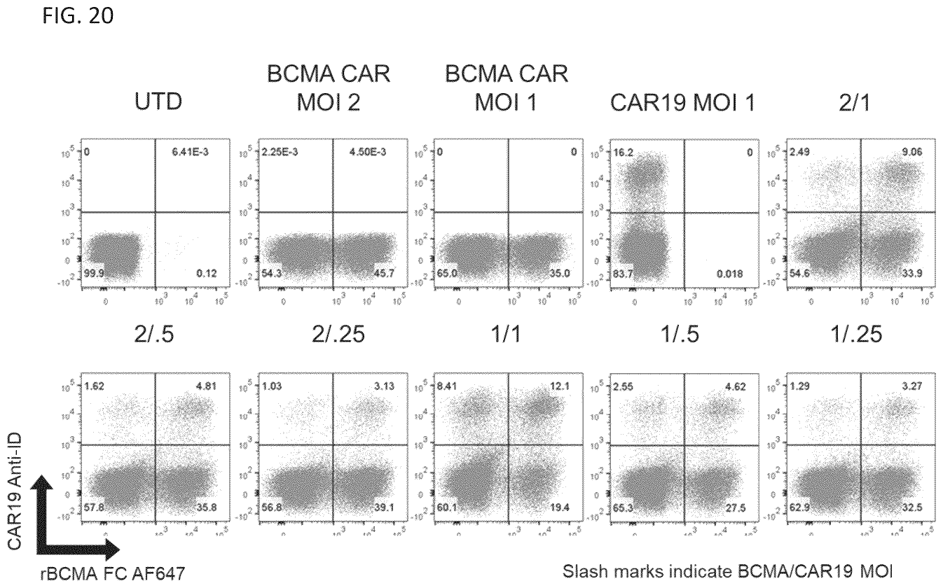

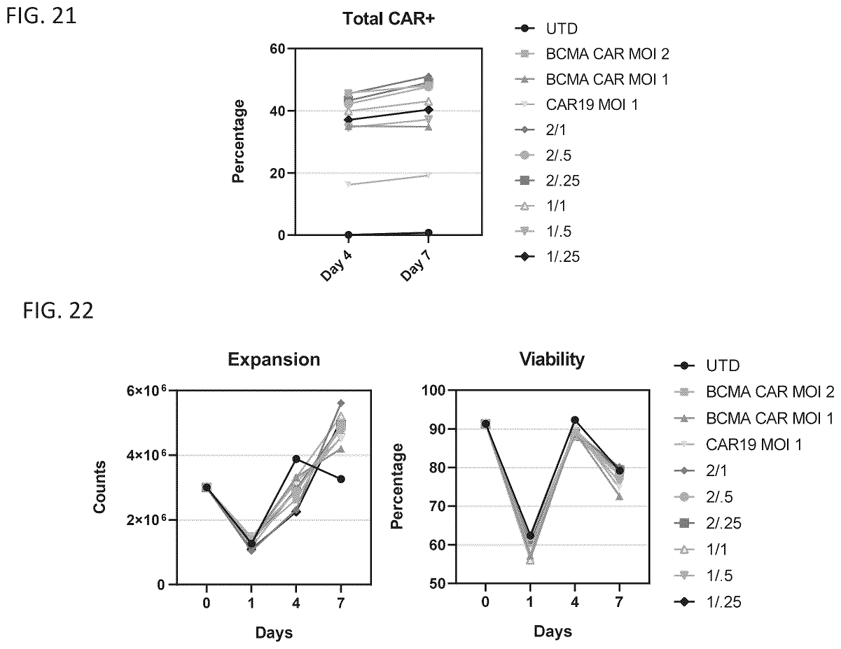

At Day four after viral addition, flow cytometry analysis showed significantly higher percentages of mono BCMA CAR+ population over the CAR19 positive population and the double CAR positive population in the BCMA CAR MOI 2 conditions (FIGS. 19 and 20). This was consistent across both donors. When P161 was added at a MOI of 1, good titration of all the populations was observed (FIGS. 19 and 20). CAR19+ population as well as the double CAR+ population decreased as the CD19 CAR MOI decreased from 1 to 0.25 (FIG. 19). The BCMA CAR+ population increased when the CTL119-encoding virus was added at a lower amount (FIG. 19). Total CAR+ populations correlated in percentages with the total MOI added to each well (FIG. 21). Total BCMA CAR and CAR19 percentages correlated as well. All trends were stable from day four to day seven (FIG. 21). Viability and expansion rates were independent of the total MOI added (FIG. 22). Data from one of the four donors are shown here.

### Example 5: Description of the Activated Rapid Manufacturing (ARM) Process

In some embodiments, CART cells are manufactured using a continuous Activated Rapid Manufacturing (ARM) process, over approximately 2 days, which will potentially allow for a greater number of less differentiated T cells (T naïve and T_{SCM} (stem central memory T) cells) to be returned to a patient for in vivo cellular expansion. The short manufacturing time period allows the early differentiated T cells profile to proliferate in the body for their desired terminal differentiated state rather that in an ex vivo culture vessel.

In some embodiments, CART cells are manufactured using cryopreserved leukapheresis source material, for example, non-mobilized autologous peripheral blood leukapheresis (LKPK) material. Cryopreserved source material undergoes processing steps for T cell enrichment on the first day of production (Day 0) by means of anti-CD4/anti-CD8 immunomagnetic system. Positive fraction is then seeded in G-rex culture vessel, activated with an anti-CD3/CD28 system (TransACT) and on the same day transduced with a lentiviral vector (LV) encoding a CAR. On the following day, after 20-28 hours of transduction, the T cells are harvested, washed four times, formulated in freezing medium and then frozen by a Controlled Rate Freezer (CRF). From the start of the process on Day 0 to the initiation of harvest on the following day, cells are cultured for 20-28 hours with a target of 24 hours after Day 0 seeding.

Media for Day 0 were prepared according to Table 21.

TABLE 21
Media type and point of use during CART manufacturing

| Media/Buffer Type | Composition | Point of Use |
|---|---|---|
| Rapid Buffer (RB) | CliniMACS ® Buffer | Day 0 Processing |
| | (+0.5% human | on Cell |
| | serum albumin (HSA)) | Wash/Separator |
| Rapid Media (RM) | OpTmizer ™ Media, | Day 0 for Processing |
| | CTS ™, IL-2, | on Cell Wash/ |
| | Glutamax and ICSR | Separator and Cell |
| | | Seeding |
| Harvest Buffer (HB) | PBS no EDTA and | Harvest Wash |
| (also called Harvest | 2% HSA | Buffer (Day 1) |
| Buffer Solution) | | |
| Cryomedia | Cryostor10 (CS10) | Harvest Formulation |

The cryopreserved leukapheresis material is thawed. The thawed cells are diluted with the Rapid Buffer (Table 21) and washed on the CliniMACS® Prodigy® device. The T cells are selected by CliniMACS® CD4 and CD8 microbeads. Once the program is finished for T cell selection (approximately 3 h 40 min to 4 h 40 min), the reapplication bag containing the cells suspended in Rapid Media (Table 21) are transferred in a transfer pack. A sample is taken for viability and cell count. The cell count and viability data from the positive fraction bag is used to determine the cell concentration when seeding the culture vessel for activation and vector transduction.

Following positive selection of T cells via the CliniMACS® microbeads (CD4 and CD8), the cells are seeded in the culture vessel, G-Rex. Once the cells are seeded, the activation reagent (TransACT) is then added to the culture vessel. The cells are then transduced with a lentiviral vector encoding a CAR at a target MOI of 1.0 (0.8-1.2). Following the vector addition, the culture vessel is transported to an incubator where it is incubated for a target of 24 hours (operating range 20-28 hours) at a nominal temperature of 37° C. (operating range 36-38° C.) with nominal 5% CO₂ (operating range 4.5-5.5%). Following the incubation, the cells are washed with Harvest Wash Solution (Table 21) four times to remove any non-integrated vector and residual viral particles, as well as any other process related impurities. Then, the cells are eluted and a sample for cell count and viability is taken for testing and the results are used to determine the volume required to re-suspend the cells for final formulation with CryoStor® CS10. The cells are then centrifugated to remove the Harvest Wash Solution and proceed with cryopreservation.

In some embodiments, the CAR expressed in CART cells binds to CD19. In some embodiments, IL-2 used in the Rapid Media (RM) (Table 21) can be replaced with IL-15, hetIL-15 (IL-15/sIL-15Ra), IL-6, or IL-6/sIL-6Ra.

In some embodiments, the CAR expressed in CART cells binds to BCMA. In some embodiments, IL-2 used in the Rapid Media (RM) (Table 21) can be replaced with IL-15, hetIL-15 (IL-15/sIL-15Ra), IL-6, or IL-6/sIL-6Ra.

In some embodiments, the CART cells express dual CARs disclosed herein, e.g., anti-BCMA/anti-CD19 dual CARs disclosed herein. In some embodiments, the CART cells express a diabody CAR disclosed herein, e.g., an anti-BCMA/anti-CD19 diabody CAR disclosed herein. In some embodiments, the CART cells are engineered to express an anti-BCMA CAR and an anti-CD19 CAR using co-transduction as disclosed herein.

### Example 6: Manufacturing CART Cells Using the Activated Rapid Manufacturing (ARM) Process

The ARM process of CART cells initiates with the preparation of the media as outlined in Table 25.

Cryopreserved leukapheresis product is used as the starting material and is processed for T cell enrichment. When available, the apheresis paperwork is utilized to define the T cell percentage. In the absence of the T cell percentage data on the apheresis paperwork, the sentinel vial testing is performed on incoming cryopreserved leukapheresis products to obtain T cell percentage target for the apheresis. The results for the T cell percentage determine how many bags are thawed on Day 0 of the ARM process.

TABLE 25
Media and Buffer type and point of use during CART manufacturing

| Media Type | Source | Point of Use |
|---|---|---|
| CliniMACS ® Buffer/ | Prepared by | Day 0 Processing |
| human serum | operator on | on Cell |
| albumin (HSA) | day 0 | Wash/Separator |
| (0.5% in working | | |
| concentration) | | |
| Rapid Media | Prepared by | Day 0 for Cell |
| | operator on day 0 | Seeding |
| PBS/HSA (1% or | Prepared by | Harvest and |
| 2% in working | operator on | culture Wash |
| concentration) | day 0 | Media (Day 1) |
| Cryostor10 (CS10) | Commercially | Harvest Formulation |
| | available | |

Cryopreserved leukapheresis is thawed, washed, and then undergoes T cell selection and enrichment using CliniMACS® microbead technology. Viable nucleated cells (VNCs) are activated with TransACT (Miltenyi) and transduced with a lentiviral vector encoding the CAR. The viable cells selected with the Miltenyi microbeads are seeded into the centricult on the Prodigy®, which is a non-humidified incubation chamber. While in culture, the cells are suspended in Rapid media, which is an OpTmizer™ CTS™ based medium that contains the CTS™ Supplement (ThermoFisher), Glutamax, IL-2 and 2% Immune cell serum replacement amongst its components to promote T cell activation and transduction. Lentiviral transduction is performed once on the day of seeding after the TransACT has been added to the diluted cells in the culture media. Lentiviral vector will be thawed immediately prior to use on day of seeding for up to 30 minutes at room temperature.

From the start of the process on Day 0 to the initiation of the culture wash and harvest, CART cells are cultured for 20-28 hours from seeding. Following culture, the cell suspension undergoes two culture washes and one harvest wash within the centricult chamber (Miltenyi Biotech).

After the harvest wash on the CliniMACS® Prodigy® on day 1, the cell suspension is sampled to determine viable cell count and viability. Cell suspension is then transferred to a centrifuge to be pelleted manually. The supernatant is removed, and the cell pellet is re-suspended in CS10 (BioLife Solution), resulting in a product formulation with a final DMSO concentration of ˜10.0%. The viable cell count is formulated at the end of harvest for dosing. The doses are then distributed into individual cryobags and analytical sampling into cryovials.

Cryopreserved products are stored in monitored LN2 storage tanks, in a secure, limited access area until final release and shipping.

In some embodiments, the CART cells express dual CARs disclosed herein, e.g., anti-BCMA/anti-CD19 dual CARs disclosed herein. In some embodiments, the CART cells express a diabody CAR disclosed herein, e.g., an anti-BCMA/anti-CD19 diabody CAR disclosed herein. In some embodiments, the CART cells are engineered to express an anti-BCMA CAR and an anti-CD19 CAR using co-transduction as disclosed herein.

### Example 7: Manufacture of CART Cells Expressing an Anti-BCMA CAR and an Anti-CD19 CAR

The rapid manufacturing process of CART cells begins with the preparation of the media as outlined in Table 33.

TABLE 33
Media type and point of use during CART manufacturing

| Media/Buffer Type | Composition | Process Step |
|---|---|---|
| Rapid Media (RM) | OpTmizer CTS | Cell Seeding |
| | CTS supplement | and activation |
| | ICSR | |
| | GlutaMAX | |
| | Reconstituted IL-2 | |
| Rapid Buffer (RB) | CliniMACs Buffer | Cell Wash and T |
| | (PBS with EDTA) | cell enrichment |
| | HSA | |
| Culture/Harvest | PBS (no Mg/Ca and | Harvest Wash |
| Wash Solution | EDTA) HSA | procedure |
| Cryomedia | CryoStor ® (CS10) | Harvest |
| | with DMSO | Formulation |

Cryopreserved leukapheresis is thawed. The thawed cells are diluted with the Rapid Buffer (Table 33) and washed on the CliniMACS® Prodigy® device. The T cells are selected by CliniMACS® CD4 and CD8 microbeads. Once the program is finished for T cell selection (approximately 3 h 40 min to 4 h 40 min), the reapplication bag will contain the cells suspended in Rapid Buffer (Table 33). A sample is taken for viability and cell count. The cell count and viability data from the positive fraction bag is used to determine the cell concentration when seeding the culture vessel for activation and vector transduction.

Following positive selection of T cells via the CliniMACS® microbeads (CD4 and CD8), the cells are seeded in the culture vessel, CentriCult in the Prodigy®, at a target of 4.0×10⁸-1.0×10⁹ total viable cells at a targeted concentration of about 4.0×10⁶ viable cells/mL. Once the cells are seeded, the activation reagent (TransAct) is then added to the culture vessel.

The cells are then transduced with a lentiviral vector encoding an anti-BCMA CAR and a lentiviral vector encoding an anti-CD19 CAR. The vector volume to be used for transduction of T cells, following positive selection, is calculated based on a target Multiplicity of Infection (MOI) of 4.75 for the BCMA CAR lentiviral vector and a target MOI of 0.5 for the CD19 CAR lentiviral vector.

After a target of 24 hours (operating range 20-28 hours) of incubation at a temperature of 37° C. with nominal 5% CO₂, the cells are processed for harvest wash.

Following the incubation, the cells are washed with Harvest Wash Solution (Table 33) three times to remove any non-integrated vector and residual viral particles, as well as any other process related impurities. Then, the cells are eluted and a sample for cell count and viability is taken for testing and the results are used to determine the volume required to re-suspend the cells for final formulation with CryoStor® CS10. The cells are then centrifugated to remove the Harvest Wash Solution and proceed with cryopreservation.

### Example 8: Gene Signature Analysis of CART Cells Manufactured Using the ARM Process

### Methods

### Single Cell RNAseq

Single cell RNAseq libraries were generated using the 10× Genomics Chromium Controller instrument and supporting library construction kits.

Cryopreserved cells were thawed, counted and flow sorted (if required for study question), prior to being loaded on a 10× Genomics Instrument. Individual cells were loaded into droplets and RNA within individual droplets was barcoded via a GemCode bead. Barcoded RNA was released from droplets and converted into a whole transcriptome Illumina compatible sequencing library.

Generated libraries were sequenced on an Illumina HiSeq Instrument and analyzed using 10× Genomics analysis pipeline and Loupe Cell Browser software.

### Single Cell Immune Cell Profiling

Whole transcriptome 10× Genomics single cell libraries were used as a template material to generate immune cell profiling and repertoire analysis. T cell receptor sequences were PCR amplified from Chromium Single Cell 5′ Libraries and analyzed on an Illumina sequencing instrument.

### Analysis Pipeline

Single cell RNAseq data was processed through the Cell Ranger analysis pipeline starting with FASTQ files. A detailed description of the Cell Ranger analysis pipeline can be found at: support 0.10xgenomics.com/single-cell-gene-expression/software/pipelines/latest/what-is-cell-ranger. The general pipeline included alignment, filtering, barcode counting, and UMI counting. Cellular barcodes were used to generate gene-barcode matrices, determine clusters, and perform gene expression analysis. Gene expression count data was normalized using the Seurat Bioconductor package. Cells were discarded from the analysis that had less than 200 expressed genes. Genes were discarded from the analysis that were only expressed in 2 cells or less. The remaining data was normalized with the Seurat log normalization method using a scale factor of 10,000. Data was scaled by regressing on the number of detected molecules per cell. The gene set score (GeneSetScore) was calculated by taking the mean log normalized gene expression value of all the genes in the gene set. Each gene is z-score normalized so that the mean expression of the gene across samples is 0 and standard deviation is 1. The gene set score is then calculated as the mean of the normalized values of the genes in the gene set. An exemplary gene set score calculation is described below.

For this example of gene set score calculation, the normalized gene expression of two (2) samples for six (6) genes is provided in Table 23. For the purposes of this exemplary calculation, the gene set consists of genes 1-4. Therefore, Sample 1 and 2 both have gene set scores of 0.

TABLE 23
Exemplary dataset for gene set score calculation

| | Sample 1 | Sample 2 |
|---|---|---|
| Gene 1 | −3 | 0 |
| Gene 2 | 3 | 0 |
| Gene 3 | 1 | 0 |
| Gene 4 | −1 | 0 |
| Gene 5 | 10 | 4 |
| Gene 6 | −5 | 3 |

The gene set “Up TEM vs. Down TSCM” includes the following genes: MXRA7, CLIC1, NAT13, TBC1D2B, GLCCI1, DUSP10, APOBEC3D, CACNB3, ANXA2P2, TPRG1, EOMES, MATK, ARHGAP10, ADAM8, MAN1A1, SLFN12L, SH2D2A, EIF2C4, CD58, MYO1F, RAB27B, ERN1, NPC1, NBEAL2, APOBEC3G, SYTL2, SLC4A4, PIK3AP1, PTGDR, MAF, PLEKHA5, ADRB2, PLXND1, GNAO1, THBS1, PPP2R2B, CYTH3, KLRF1, FLJ16686, AUTS2, PTPRM, GNLY, and GFPT2.

The gene set “Up Treg vs. Down Teff” includes the following genes: C12orf75, SELPLG, SWAP70, RGS1, PRR11, SPATS2L, SPATS2L, TSHR, C14orf145, CASP8, SYT11, ACTN4, ANXA5, GLRX, HLA-DMB, PMCH, RAB11FIP1, IL32, FAM160B1, SHMT2, FRMD4B, CCR3, TNFRSF13B, NTNG2, CLDND1, BARD1, FCER1G, TYMS, ATP1B1, GJB6, FGL2, TK1, SLC2A8, CDKN2A, SKAP2, GPR55, CDCA7, S100A4, GDPD5, PMAIP1, ACOT9, CEP55, SGMS1, ADPRH, AKAP2, HDAC9, IKZF4, CARD17, VAV3, OBFC2A, ITGB1, CIITA, SETD7, HLA-DMA, CCR10, KIAA0101, SLC14A1, PTTG3P, DUSP10, FAM164A, PYHIN1, MYO1F, SLC1A4, MYBL2, PTTG1, RRM2, TP53INP1, CCR5, ST8SIA6, TOX, BFSP2, ITPRIPL1, NCAPH, HLA-DPB2, SYT4, NINJ2, FAM46C, CCR4, GBP5, C15orf53, LMCD1, MKI67, NUSAP1, PDE4A, E2F2, CD58, ARHGEF12, LOC100188949, FAS, HLA-DPB1, SELP, WEE1, HLA-DPA1, FCRL1, ICA1, CNTNAP1, OAS1, METTL7A, CCR6, HLA-DRB4, ANXA2P3, STAM, HLA-DQB2, LGALS1, ANXA2, PI16, DUSP4, LAYN, ANXA2P2, PTPLA, ANXA2P1, ZNF365, LAIR2, LOC541471, RASGRP4, BCAS1, UTS2, MIAT, PRDM1, SEMA3G, FAM129A, HPGD, NCF4, LGALS3, CEACAM4, JAKMIP1, TIGIT, HLA-DRA, IKZF2, HLA-DRB1, FANK1, RTKN2, TRIB1, FCRL3, and FOXP3.

The gene set “Down stemness” includes the following genes: ACE, BATF, CDK6, CHD2, ERCC2, HOXB4, MEOX1, SFRP1, SP7, SRF, TAL1, and XRCC5.

The gene set “Up hypoxia” includes the following genes: ABCB1, ACAT1, ADM, ADORA2B, AK2, AK3, ALDH1A1, ALDH1A3, ALDOA, ALDOC, ANGPT2, ANGPTL4, ANXA1, ANXA2, ANXA5, ARHGAP5, ARSE, ART1, BACE2, BATF3, BCL2L1, BCL2L2, BHLHE40, BHLHE41, BIK, BIRC2, BNIP3, BNIP3L, BPI, BTG1, C11orf2, C7orf68, CA12, CA9, CALD1, CCNG2, CCT6A, CD99, CDK1, CDKN1A, CDKN1B, CITED2, CLK1, CNOT7, COL4A5, COL5A1, COL5A2, COL5A3, CP, CTSD, CXCR4, D4S234E, DDIT3, DDIT4, 1-Dec, DKC1, DR1, EDN1, EDN2, EFNA1, EGF, EGR1, EIF4A3, ELF3, ELL2, ENG, ENO1, ENO3, ENPEP, EPO, ERRFI1, ETS1, F3, FABP5, FGF3, FKBP4, FLT1, FN1, FOS, FTL, GAPDH, GBE1, GLRX, GPI, GPRC5A, HAP1, HBP1, HDAC1, HDAC9, HERC3, HERPUD1, HGF, HIF1A, HK1, HK2, HLA-DQB1, HMOX1, HMOX2, HSPA5, HSPD1, HSPH1, HYOU1, ICAM1, ID2, IFI27, IGF2, IGFBP1, IGFBP2, IGFBP3, IGFBP5, IL6, IL8, INSIG1, IRF6, ITGA5, JUN, KDR, KRT14, KRT18, KRT19, LDHA, LDHB, LEP, LGALS1, LONP1, LOX, LRP1, MAP4, MET, MIF, MMP13, MMP2, MMP7, MPI, MT1L, MTL3P, MUC1, MXIl, NDRG1, NFIL3, NFKB1, NFKB2, NOS1, NOS2, NOS2P1, NOS2P2, NOS3, NR3C1, NR4A1, NT5E, ODC1, P4HA1, P4HA2, PAICS, PDGFB, PDK3, PFKFB1, PFKFB3, PFKFB4, PFKL, PGAM1, PGF, PGK1, PGK2, PGM1, PIM1, PIM2, PKM2, PLAU, PLAUR, PLIN2, PLOD2, PNN, PNP, POLM, PPARA, PPAT, PROK1, PSMA3, PSMD9, PTGS1, PTGS2, QSOX1, RBPJ, RELA, RIOK3, RNASEL, RPL36A, RRP9, SAT1, SERPINB2, SERPINE1, SGSM2, SIAH2, SIN3A, SIRPA, SLC16A1, SLC16A2, SLC20A1, SLC2A1, SLC2A3, SLC3A2, SLC6A10P, SLC6A16, SLC6A6, SLC6A8, SORL1, SPP1, SRSF6, SSSCA1, STC2, STRA13, SYT7, TBPL1, TCEAL1, TEK, TF, TFF3, TFRC, TGFA, TGFB1, TGFB3, TGFBI, TGM2, TH, THBS1, THBS2, TIMM17A, TNFAIP3, TP53, TPBG, TPD52, TPI1, TXN, TXNIP, UMPS, VEGFA, VEGFB, VEGFC, VIM, VPS11, and XRCC6.

The gene set “Up autophagy” includes the following genes: ABL1, ACBD5, ACIN1, ACTRT1, ADAMTS7, AKR1E2, ALKBH5, ALPK1, AMBRA1, ANXA5, ANXA7, ARSB, ASB2, ATG10, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATP13A2, ATP1B1, ATPAF1-AS1, ATPIF1, BECN1, BECN1P1, BLOC1S1, BMP2KL, BNIP1, BNIP3, BOC, C11orf2, C11orf4l, C12orf44, C12orf5, C14orf133, C1orf210, C5, C6orf106, C7orf59, C7orf68, C8orf59, C9orf72, CA7, CALCB, CALCOCO2, CAPS, CCDC36, CD163L1, CD93, CDC37, CDKN2A, CHAF1B, CHMP2A, CHMP2B, CHMP3, CHMP4A, CHMP4B, CHMP4C, CHMP6, CHST3, CISD2, CLDN7, CLEC16A, CLN3, CLVS1, COX8A, CPA3, CRNKL1, CSPG5, CTSA, CTSB, CTSD, CXCR7, DAP, DKKL1, DNAAF2, DPF3, DRAM1, DRAM2, DYNLL1, DYNLL2, DZANK1, E124, EIF2S1, EPG5, EPM2A, FABP1, FAM125A, FAM131B, FAM134B, FAM13B, FAM176A, FAM176B, FAM48A, FANCC, FANCF, FANCL, FBXO7, FCGR3B, FGF14, FGF7, FGFBP1, FIS1, FNBP1L, FOXO1, FUNDC1, FUNDC2, FXR2, GABARAP, GABARAPL1, GABARAPL2, GABARAPL3, GABRA5, GDF5, GMIP, HAP1, HAPLN1, HBXIP, HCAR1, HDAC6, HGS, HIST1H3A, HIST1H3B, HIST1H3C, HIST1H3D, HIST1H3E, HIST1H3F, HIST1H3G, HIST1H3H, HIST1H3I, HIST1H3J, HK2, HMGB1, HPR, HSF2BP, HSP90AA1, HSPA8, IFI16, IPPK, IRGM, IST1, ITGB4, ITPKC, KCNK3, KCNQ1, KIAA0226, KIAA1324, KRCC1, KRT15, KRT73, LAMP1, LAMP2, LAMTOR1, LAMTOR2, LAMTOR3, LARP1B, LENG9, LGALS8, LIX1, LIX1L, LMCD1, LRRK2, LRSAM1, LSM4, MAP1A, MAP1LC3A, MAP1LC3B, MAP1LC3B2, MAP1LC3C, MAP1S, MAP2K1, MAP3K12, MARK2, MBD5, MDH1, MEX3C, MFN1, MFN2, MLST8, MRPS10, MRPS2, MSTN, MTERFD1, MTMR14, MTMR3, MTOR, MTSS1, MYH11, MYLK, MYOM1, NBR1, NDUFB9, NEFM, NHLRC1, NME2, NPC1, NR2C2, NRBF2, NTHL1, NUP93, OBSCN, OPTN, P2RX5, PACS2, PARK2, PARK7, PDK1, PDK4, PEX13, PEX3, PFKP, PGK2, PHF23, PHYHIP, PI4K2A, PIK3C3, PIK3CA, PIK3CB, PIK3R4, PINK1, PLEKHM1, PLOD2, PNPO, PPARGC1A, PPY, PRKAA1, PRKAA2, PRKAB1, PRKAB2, PRKAG1, PRKAG2, PRKAG3, PRKD2, PRKG1, PSEN1, PTPN22, RAB12, RAB1A, RAB1B, RAB23, RAB24, RAB33B, RAB39, RAB7A, RB1CC1, RBM18, REEP2, REP15, RFWD3, RGS19, RHEB, RIMS3, RNF185, RNF41, RPS27A, RPTOR, RRAGA, RRAGB, RRAGC, RRAGD, S100A8, S100A9, SCN1A, SERPINB10, SESN2, SFRP4, SH3GLB1, SIRT2, SLC1A3, SLC1A4, SLC22A3, SLC25A19, SLC35B3, SLC35C1, SLC37A4, SLC6A1, SLCO1A2, SMURF1, SNAP29, SNAPIN, SNF8, SNRPB, SNRPB2, SNRPD1, SNRPF, SNTG1, SNX14, SPATA18, SQSTM1, SRPX, STAM, STAM2, STAT2, STBD1, STK11, STK32A, STOM, STX12, STX17, SUPT3H, TBC1D17, TBC1D25, TBC1D5, TCIRG1, TEAD4, TECPR1, TECPR2, TFEB, TM9SF1, TMBIM6, TMEM203, TMEM208, TMEM39A, TMEM39B, TMEM59, TMEM74, TMEM93, TNIK, TOLLIP, TOMM20, TOMM22, TOMM40, TOMM5, TOMM6, TOMM7, TOMM70A, TP53INP1, TP53INP2, TRAPPC8, TREM1, TRIM17, TRIMS, TSG101, TXLNA, UBA52, UBB, UBC, UBQLN1, UBQLN2, UBQLN4, ULK1, ULK2, ULK3, USP10, USP13, USP30, UVRAG, VAMP7, VAMP8, VDAC1, VMP1, VPS11, VPS16, VPS18, VPS25, VPS28, VPS33A, VPS33B, VPS36, VPS37A, VPS37B, VPS37C, VPS37D, VPS39, VPS41, VPS4A, VPS4B, VTA1, VTI1A, VTI1B, WDFY3, WDR45, WDR45L, WIPI1, WIPI2, XBP1, YIPF1, ZCCHC17, ZFYVE1, ZKSCAN3, ZNF189, ZNF593, and ZNF681.

The gene set “Up resting vs. Down activated” includes the following genes: ABCA7, ABCF3, ACAP2, AMT, ANKH, ATF7IP2, ATG14, ATP1A1, ATXN7, ATXN7L3B, BCL7A, BEX4, BSDC1, BTG1, BTG2, BTN3A1, C11orf21, C19orf22, C21orf2, CAMK2G, CARS2, CCNL2, CD248, CD5, CD55, CEP164, CHKB, CLK1, CLK4, CTSL1, DBP, DCUN1D2, DENND1C, DGKD, DLG1, DUSP1, EAPP, ECE1, ECHDC2, ERBB2IP, FAM117A, FAM134B, FAM134C, FAM169A, FAM190B, FAU, FLJ10038, FOXJ2, FOXJ3, FOXL1, FOXO1, FXYD5, FYB, HLA-E, HSPA1L, HYAL2, ICAM2, IFIT5, IFITM1, IKBKB, IQSEC1, IRS4, KIAA0664L3, KIAA0748, KLF3, KLF9, KRT18, LEF1, LINC00342, LIPA, LIPT1, LLGL2, LMBR1L, LPAR2, LTBP3, LYPD3, LZTFL1, MANBA, MAP2K6, MAP3K1, MARCH8, MAU2, MGEA5, MMP8, MPO, MSL1, MSL3, MYH3, MYLIP, NAGPA, NDST2, NISCH, NKTR, NLRP1, NOSIP, NPIP, NUMA1, PAIP2B, PAPD7, PBXIP1, PCIF1, PI4KA, PLCL2, PLEKHA1, PLEKHF2, PNISR, PPFIBP2, PRKCA, PRKCZ, PRKD3, PRMT2, PTP4A3, PXN, RASA2, RASA3, RASGRP2, RBM38, REPIN1, RNF38, RNF44, ROR1, RPL30, RPL32, RPLP1, RPS20, RPS24, RPS27, RPS6, RPS9, RXRA, RYK, SCAND2, SEMA4C, SETD1B, SETD6, SETX, SF3B1, SH2B1, SLC2A4RG, SLC35E2B, SLC46A3, SMAGP, SMARCE1, SMPD1, SNPH, SP140L, SPATA6, SPG7, SREK1IP1, SRSF5, STAT5B, SVIL, SYF2, SYNJ2BP, TAF1C, TBC1D4, TCF20, TECTA, TES, TMEM127, TMEM159, TMEM30B, TMEM66, TMEM8B, TP53 TG1, TPCN1, TRIM22, TRIM44, TSC1, TSC22D1, TSC22D3, TSPYL2, TTC9, TTN, UBE2G2, USP33, USP34, VAMP1, VILL, VIPR1, VPS13C, ZBED5, ZBTB25, ZBTB40, ZC3H3, ZFP161, ZFP36L1, ZFP36L2, ZHX2, ZMYM5, ZNF136, ZNF148, ZNF318, ZNF350, ZNF512B, ZNF609, ZNF652, ZNF83, ZNF862, and ZNF91.

The gene set “Progressively up in memory differentiation” includes the following genes: MTCH2, RAB6C, KIAA0195, SETD2, C2orf24, NRD1, GNA13, COPA, SELT, TNIP1, CBFA2 T2, LRP10, PRKCI, BRE, ANKS1A, PNPLA6, ARL6IP1, WDFY1, MAPK1, GPR153, SHKBP1, MAP1LC3B2, PIP4K2A, HCN3, GTPBP1, TLN1, C4orf34, KIF3B, TCIRG1, PPP3CA, ATG4D, TYMP, TRAF6, C17orf76, WIPF1, FAM108A1, MYL6, NRM, SPCS2, GGT3P, GALK1, CLIP4, ARL4C, YWHAQ, LPCAT4, ATG2A, IDS, TBC1D5, DMPK, ST6GALNAC6, REEP5, ABHD6, KIAA0247, EMB, TSEN54, SPIRE2, PIWIL4, ZSCAN22, ICAM1, CHD9, LPIN2, SETD8, ZC3H12A, ULBP3, IL15RA, HLA-DQB2, LCP1, CHP, RUNX3, TMEM43, REEP4, MEF2D, ABL1, TMEM39A, PCBP4, PLCD1, CHST12, RASGRP1, C1orf58, C11orf63, C6orf129, FHOD1, DKFZp434F142, PIK3CG, ITPR3, BTG3, C4orf50, CNNM3, IFI16, AK1, CDK2AP1, REL, BCL2L1, MVD, TTC39C, PLEKHA2, FKBP11, EML4, FANCA, CDCA4, FUCA2, MFSD10, TBCD, CAPN2, IQGAP1, CHST11, PIK3R1, MYOSA, KIR2DL3, DLG3, MXD4, RALGDS, S1PR5, WSB2, CCR3, TIPARP, SP140, CD151, SOX13, KRTAP5-2, NF1, PEA15, PARP8, RNF166, UEVLD, LIMK1, CACNB1, TMX4, SLC6A6, LBA1, SV2A, LLGL2, IRF1, PPP2R5C, CD99, RAPGEF1, PPP4R1, OSBPL7, FOXP4, SLA2, TBC1D2B, ST7, JAZF1, GGA2, PI4K2A, CD68, LPGAT1, STX11, ZAK, FAM160B1, RORA, C8orf80, APOBEC3F, TGFBI, DNAJC1, GPR114, LRP8, CD69, CMIP, NAT13, TGFB1, FLJ00049, ANTXR2, NR4A3, IL12RB1, NTNG2, RDX, MLLT4, GPRIN3, ADCY9, CD300A, SCD5, ABI3, PTPN22, LGALS1, SYTL3, BMPR1A, TBK1, PMAIP1, RASGEF1A, GCNT1, GABARAPL1, STOM, CALHM2, ABCA2, PPP1R16B, SYNE2, PAM, C12orf75, CLCF1, MXRA7, APOBEC3C, CLSTN3, ACOT9, HIP1, LAG3, TNFAIP3, DCBLD1, KLF6, CACNB3, RNF19A, RAB27A, FADS3, DLG5, APOBEC3D, TNFRSF1B, ACTN4, TBKBP1, ATXN1, ARAP2, ARHGEF12, FAM53B, MAN1A1, FAM38A, PLXNC1, GRLF1, SRGN, HLA-DRB5, B4GALT5, WIPI1, PTPRJ, SLFN11, DUSP2, ANXA5, AHNAK, NEO1, CLIC1, EIF2C4, MAP3K5, IL2RB, PLEKHG1, MYO6, GTDC1, EDARADD, GALM, TARP, ADAM8, MSC, HNRPLL, SYT11, ATP2B4, NHSL2, MATK, ARHGAP18, SLFN12L, SPATS2L, RAB27B, PIK3R3, TP53INP1, MBOAT1, GYGI, KATNAL1, FAM46C, ZC3HAV1L, ANXA2P2, CTNNA1, NPC1, C3AR1, CRIM1, SH2D2A, ERN1, YPEL1, TBX21, SLC1A4, FASLG, PHACTR2, GALNT3, ADRB2, PIK3AP1, TLR3, PLEKHA5, DUSP10, GNAO1, PTGDR, FRMD4B, ANXA2, EOMES, CADM1, MAF, TPRG1, NBEAL2, PPP2R2B, PELO, SLC4A4, KLRF1, FOSL2, RGS2, TGFBR3, PRF1, MYO1F, GAB3, C17orf66, MICAL2, CYTH3, TOX, HLA-DRA, SYNE1, WEE1, PYHIN1, F2R, PLD1, THBS1, CD58, FAS, NETO2, CXCR6, ST6GALNAC2, DUSP4, AUTS2, C1orf2l, KLRG1, TNIP3, GZMA, PRR5L, PRDM1, ST8SIA6, PLXND1, PTPRM, GFPT2, MYBL1, SLAMF7, FLJ16686, GNLY, ZEB2, CST7, IL18RAP, CCL5, KLRD1, and KLRB1.

The gene set “Up TEM vs. Down TN” includes the following genes: MYOSA, MXD4, STK3, S1PR5, GLCCI1, CCR3, SOX13, KRTAP5-2, PEA15, PARP8, RNF166, UEVLD, LIMK1, SLC6A6, SV2A, KPNA2, OSBPL7, ST7, GGA2, PI4K2A, CD68, ZAK, RORA, TGFBI, DNAJC1, JOSD1, ZFYVE28, LRP8, OSBPL3, CMIP, NAT13, TGFB1, ANTXR2, NR4A3, RDX, ADCY9, CHN1, CD300A, SCD5, PTPN22, LGALS1, RASGEF1A, GCNT1, GLUL, ABCA2, CLDND1, PAM, CLCF1, MXRA7, CLSTN3, ACOT9, METRNL, BMPR1A, LRIG1, APOBEC3G, CACNB3, RNF19A, RAB27A, FADS3, ACTN4, TBKBP1, FAM53B, MAN1A1, FAM38A, GRLF1, B4GALT5, WIPI1, DUSP2, ANXA5, AHNAK, CLIC1, MAP3K5, ST8SIA1, TARP, ADAM8, MATK, SLFN12L, PIK3R3, FAM46C, ANXA2P2, CTNNA1, NPC1, SH2D2A, ERN1, YPEL1, TBX21, STOM, PHACTR2, GBP5, ADRB2, PIK3AP1, DUSP10, PTGDR, EOMES, MAF, TPRG1, NBEAL2, NCAPH, SLC4A4, FOSL2, RGS2, TGFBR3, MYO1F, C17orf66, CYTH3, WEE1, PYHIN1, F2R, THBS1, CD58, AUTS2, FAM129A, TNIP3, GZMA, PRR5L, PRDM1, PLXND1, PTPRM, GFPT2, MYBL1, SLAMF7, ZEB2, CST7, CCL5, GZMK, and KLRB1.

Other gene sets describing similar processes and/or characteristics can also be used to characterize cell phenotypes described above.

Cell Ranger VDJ was used to generate single cell VDJ sequences and annotations for each single cell 5′ library. Loupe Cell Browser software and Bioconductor packages were used for data analysis and visualization.

### Results

This example aims to compare T cell states between purified T cells which served as input cells, CART cells manufactured using the ARM process (labeled as “Day 1” cells), and CART cells manufactured using the TM process (labeled as “Day 9” cells) using single-cell RNA-seq (scRNA-seq). In addition, single-cell TCR-seq (scTCR-seq) was performed to study clonality and track cell differentiation from input to post-manufacturing materials.

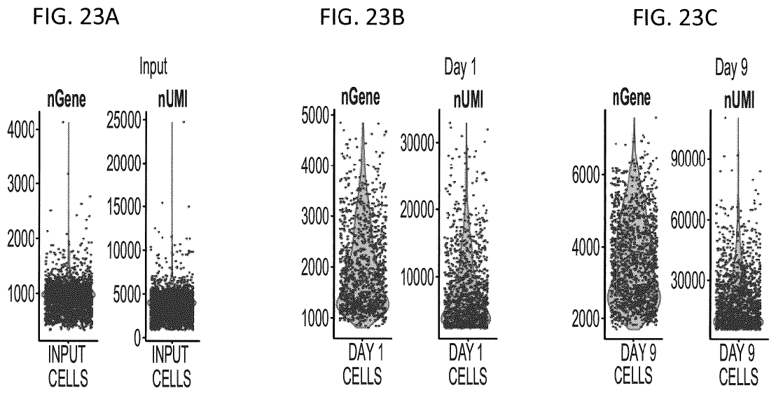

As shown in FIGS. 23A-23C, input cells had the fewest expressed genes and UMIs, suggesting these cells were not transcriptionally active and were in a resting state. Day 1 and Day 9 cells were expressing more genes, with Day 9 cells being the most transcriptionally active. Similar results are shown in FIGS. 24A-24D. Input cells were not expressing proliferation genes (FIGS. 24A and 24D).

Additional gene set analysis data are shown in FIGS. 25A-25E. Different populations of cells were compared using the median gene set scores. Day 1 cells and input cells were in a younger, more stem-like memory state (FIGS. 25A-25C). In FIG. 25A, the median GeneSetScore (Up TEM vs. Down TSCM) values for Day 1 cells, Day 9 cells, and input cells are −0.084, 0.035, and −0.1, respectively. In FIG. 25B, the median GeneSetScore (Up Treg vs. Down Teff) values for Day 1 cells, Day 9 cells, and input cells are −0.082, 0.087, and −0.071, respectively. In FIG. 25C, the median GeneSetScore (Down stemness) values for Day 1 cells, Day 9 cells, and input cells are −0.062, 0.14, and −0.081, respectively.

In addition, Day 1 cells were in a more ideal metabolic state compared to Day 9 cells (FIGS. 25D and 25E). In FIG. 25D, the median GeneSetScore (Up hypoxia) values for Day 1 cells, Day 9 cells, and input cells are 0.019, 0.11, and −0.096, respectively. In FIG. 25E, the median GeneSetScore (Up autophagy) values for Day 1 cells, Day 9 cells, and input cells are 0.066, 0.11, and −0.09, respectively.

Based on gene expression, the input cells contain four clusters. Cluster 0 is characterized by high expression of LMNA, S100A4, etc. Cluster 1 is characterized by high expression of RP913, PRKCQ-AS1, etc. Cluster 2 is characterized by high expression of PR11-291B21.2, CD8B, etc. Cluster 3 is characterized by high expression of NKG7, GZMH, CCL5, CST7, GNLY, FGFBP2, GZMA, CCL4, CTSW, CD8A, etc. In a T-Distributed Stochastic Neighbor Embedding (TSNE) plot for the input cells, Cluster 3 stood out from the other cells, and Cluster 1 and Cluster 2 were hard to differentiate.

According to the gene set analysis shown in FIGS. 26A-26C, Cluster 0 and Cluster 3 were enriched for a T regulatory phenotype compared to Cluster 1 and Cluster 2 which were enriched for a T effector phenotype. Cluster 3 was dominated by late memory/effector memory (TEM) cells, Cluster 1 and Cluster 2 were early memory and naïve cells, and Cluster 0 is in the middle. The majority of the input cells were in an early memory, naïve state. Without wishing to be bound by theory, these cells may do the best during the manufacturing procedure.

Less transcriptional heterogeneity was seen in Day 1 cells and Day 9 cells (data not shown).

Like the input population, Day 1 cells showed a large cluster of early memory cells and a smaller cluster of late memory cells in a TSNE plot. Similar to what was seen with Cluster 3 of the input cells. In contrast, Day 9 cells did not show distinct clusters of early memory cells in a TSNE plot. This implies that by day 9, the cells had become more homogeneous.

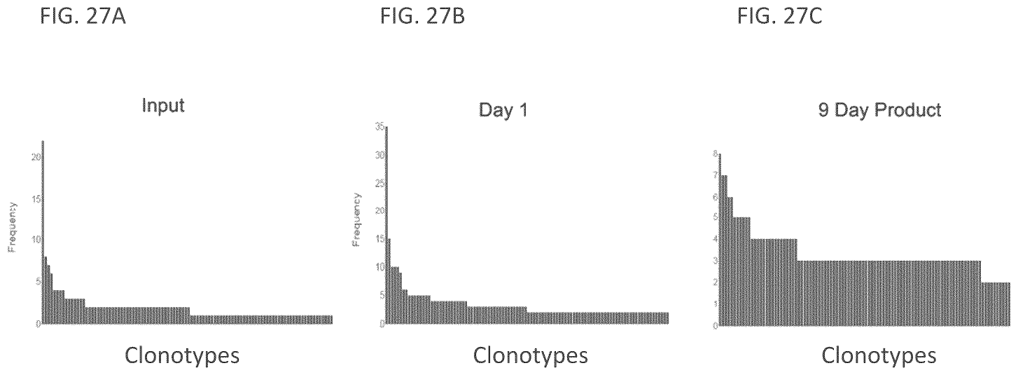

TCRs were sequenced and clonotype diversity was measured. Overall, the three clonotype profiles were very flat—most clones were only picked up once (FIGS. 27A-27C and Table 24). Shannon entropy in Table 24 measures the flatness of the distribution. The dominant clones in the input cells were late memory cells. Day 1 cells looked similar to the input cells but started to even out. By day 9, the dominate clones had substantially evened out and the distribution was much more flat. The diversity measurement was the highest at day 9 because there was a much more even and flat distribution in Day 9 cells than in the input cells or Day 1 cells.

TABLE 24
Measurements of TCR diversity

| | Input | Day 1 product | Day 9 product |
|---|---|---|---|
| Average clones per | 1.10 | 1.05 | 1.07 |
| clonotype | | | |
| Estimated number | 7344 | 7687 | 7233 |
| of cells | | | |
| Total number of | 5325 | 7403 | 6736 |
| clonotypes | | | |
| Diversity | 342.27 | 802.94 | 3382.62 |
| Normalized Shannon | 9.98E−01 | 9.95E−01 | 9.96E−01 |
| entropy | | | |

### SUMMARY

There were significant T cell state differences between Day 1 and Day 9 products. Day 1 cells were much more similar to input cells and had enrichment for stemness signatures, indicating a more efficacious product.

### Example 9: Co-Transduction of BCMA CAR and CD19 CAR, Evaluation of MOI, and Efficacy Studies

This example describes characterization of CART cells generated by co-transducing T cells with a lentiviral vector encoding a BCMA CAR and a lentiviral vector encoding a CD19 CAR. The BCMA CAR comprises the amino acid sequence of SEQ ID NO: 107 and the CD19 CAR comprises the amino acid sequence of SEQ ID NO: 225.

### Cell Preparation and CAR Transduction

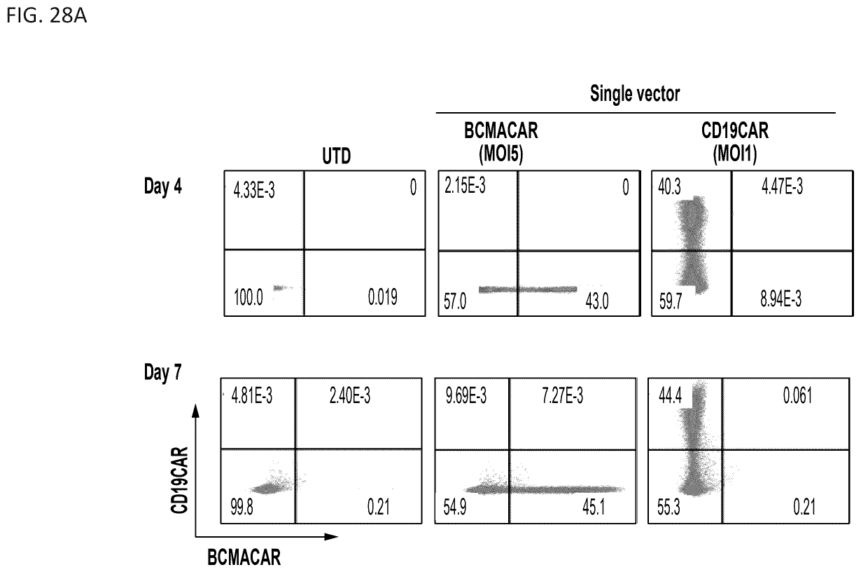

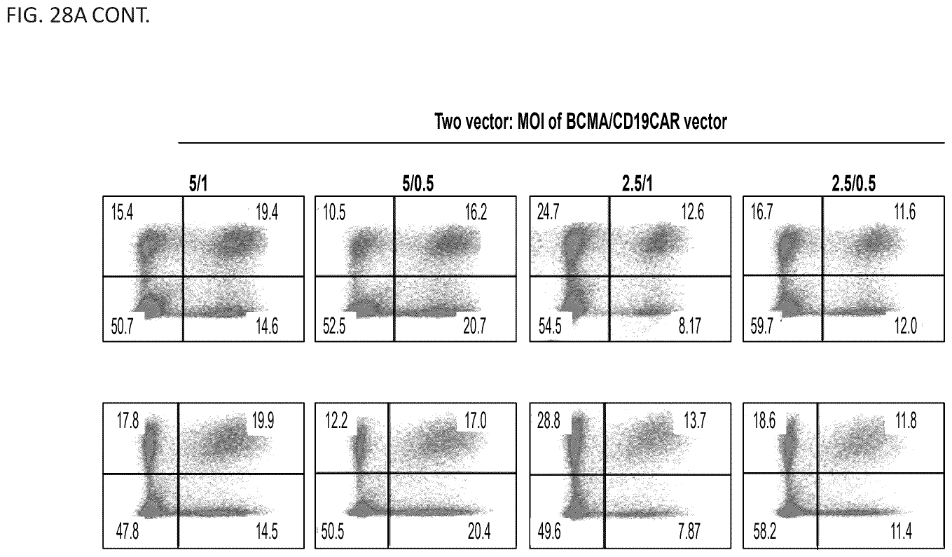

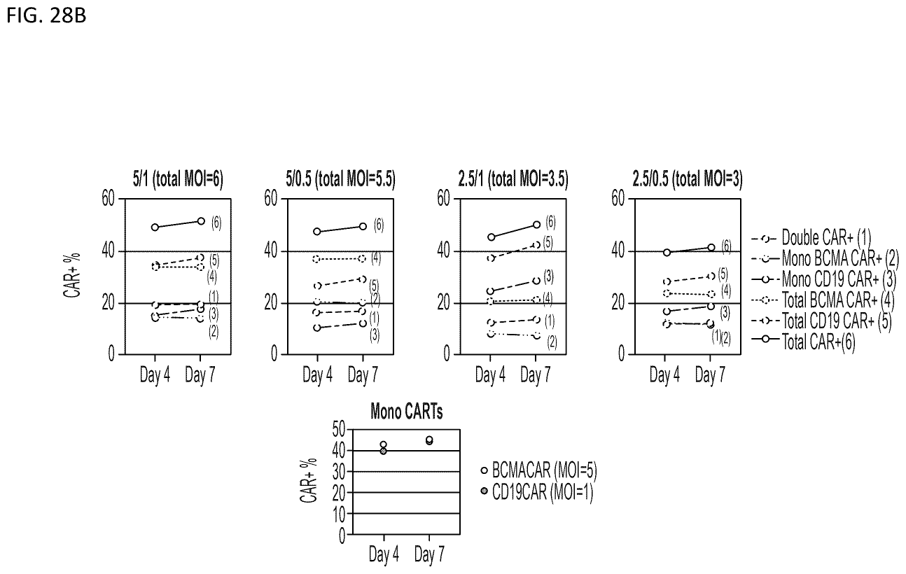

Four different MOI combinations of BCMA and CD19 CAR in transducing T cells were tested in a 24-well plate format. The BCMA CAR lentiviral vector and CD19 CAR lentiviral vector were used at MOIs of 5 and 1, respectively (5/1 in FIGS. 28A and 28B); 5 and 0.5, respectively (5/0.5 in FIGS. 28A and 28B); 2.5 and 1, respectively (2.5/1 in FIGS. 28A and 28B); or 2.5 and 0.5, respectively (2.5/0.5 in FIGS. 28A and 28B). T cells purified by Prodigy were seeded at 3 e⁶/mL, 1 mL/well into 24-well plates and both BCMA and CD19 CAR vectors were added at different MOIs as indicated above. Upon seeding, TransAct (Miltenyi Biotec), a polymeric nanomatrix conjugated to anti-CD3 and anti-CD28 agonist, was added. Cells were incubated in OpTmizer complete T cell media containing 100 IU/mL human recombinant IL-2 (Prometheus, San Diego, CA) and 2% ICRS (Life Technologies) at 37° C. and 5% CO₂ for 24 hours prior to harvest.

Cells were then washed three times with 3× volume of PBS+1% HAS. After cultivation and harvest wash, viability and cell count were assessed to determine the impact of vector titration on the final product.

Cells were then re-cultured at 1×10⁶ VNC/mL in a 24-well plate and incubated for 6 additional days. FACS analysis to assess anti-BCMA CAR expression was performed at days 4 and 7 post-transduction (3 and 6 days post-harvest).

### CAR Expression Analysis

Samples were measured on a flow cytometer (BD LSRFortessa), and data were analyzed with FlowJo software. With ARM process, as CAR may not be fully integrated and expressed ex vivo within 24 h, 96 h post viral addition could serve as a surrogate time point for in vitro and in vivo dosing strategy when the CAR is being stably expressed. The same strategy was adopted for dual targeting cocktail CAR measurements.

FACS analyses indicated that co-transduction of BCMA and CD19 CAR in T cells resulted in three distinct CAR+ subpopulations, and their proportions vary at the tested four different MOI combinations (FIG. 28A), indicating a cocktail CAR-T product could be successfully generated by co-transduction of BCMA and CD19 CAR. At an MOI of 5/0.5, mono anti-BCMA CAR+ (anti-CD19 CAR negative) was highest with 20.7%, followed by double+ CAR at 16.2% and mono anti-CD19 CAR+ at 10.5%. Total BCMA CAR+ calculated as double+ CAR % plus mono anti-BCMA CAR+ was 36.9%, while total CD19 CAR+ was 26.7% (FIGS. 28A and 28B). Both 2.5/0.5 and 2.5/1 MOI ratios resulted in much more mono CD19 CAR+% than double+% and mono anti-BCMA CAR+%. Moreover, double+ CAR % is positively correlated with total MOI usage (FIG. 28B). Total CAR+% is similar between MOI 5/1 and MOI 5/0.5. CAR expression in each population was observed to be relatively stable from day 4 to day 7 post-transduction, including BCMA or CD19 CAR transduced T cells (FIGS. 28A and 28B). Data was consistent among three donors. In some embodiments, a MOI 4-5 of BCMA CAR and a MOI 0.5 of CD19 CAR for co-transduction are used to generate dual targeting cocktail CART product.

In addition, large-scale run experiments were conducted entirely on the CliniMACS Prodigy from T cell enrichment, T cell seeding, activation, transduction and cultivation, to harvest washes in Centricult prior to formulation and cryopreservation. A MOI of 4/0.5 or 4.75/0.5 of BCMA/CD19CAR was used. This study used a seeding density of 3 e⁶/mL in a total of 250 mL for 7.5 e⁸ total cells. The cells were harvested at 24 hr, washed 3× with PBS+1% HAS and cryopreserved for downstream application. The remaining T cells were collected to generate respective control groups as UTD, BCMACART and CD19CART. An aliquot was taken and re-cultured at 1 e⁶/mL in a 24 well plate for flow cytometry analyses to assess BCMA-CAR expression at day 4 post-transduction.

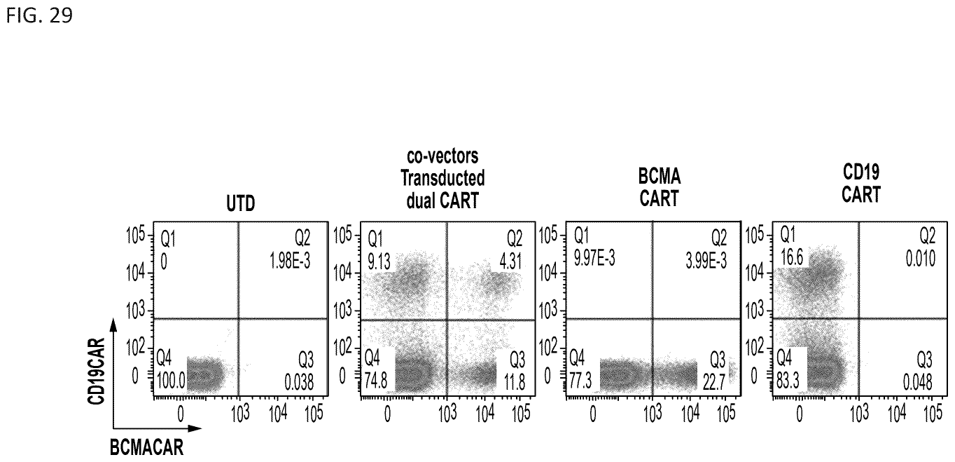

FIG. 29 showed that dual targeting cocktail CART contained 11.8% of mono anti-BCMA CART cells, 4.31% of double CART cells and ˜9% of mono anti-CD19 CART cells on day 4 post-transduction. In this study, total anti-BCMA CAR+% calculated as double CAR+% plus mono anti-BCMA CAR+% was 16%; while total anti-CD19 CAR+% calculated as double CAR+% plus mono anti-CD19 CAR+% was 13.4%.

The dual cocktail CART in vivo activity was analyzed in three xenograft mouse models: 1) a KMS-11-luc model of multiple myeloma (this BCMA-expressing model is tagged with a luciferase reporter construct, which allows the disease to be monitored systemically in the bone marrow via bioluminescent imaging); 2) a mixed tumor model established by mixing 95% of KMS-11-luc myeloma cells with 5% of CD19+ tumors (e.g., Nalm-6-luc) to mimic heterogeneity of MM patients; and 3) a Nalm-6-luc model to evaluate the specificity of CD19 targeting and additional expansion of the double positive CART population of dual targeting cocktail CART. BCMACART and CD19CART served as controls for their respective models. For CAR-T cell dose calculation, the total anti-BCMA CAR+% was measured on day 4 post-transduction for models 1 and 2, while the total anti-CD19 CAR+% was measured on day 4 post-transduction for model 3. The UTD dose reflected the highest total T cell dose of the respective process.

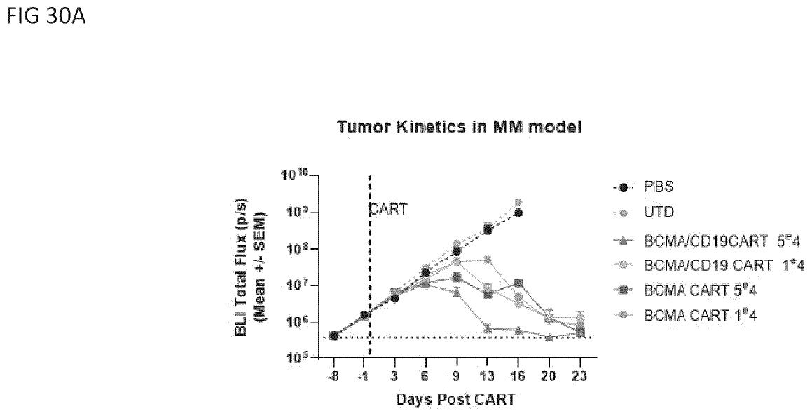

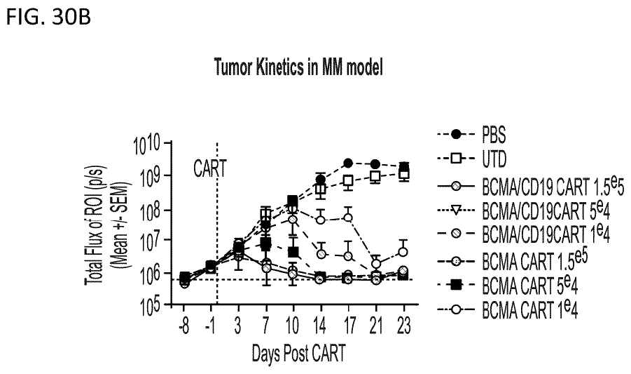

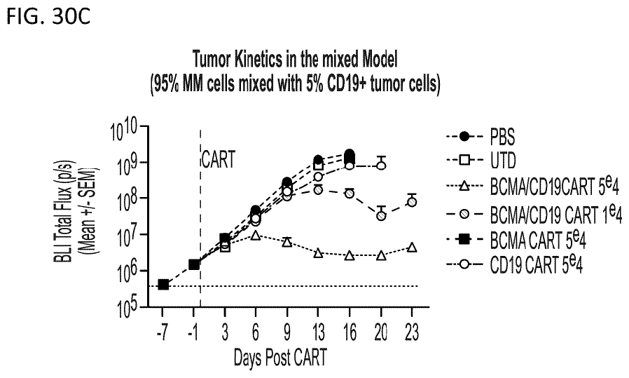

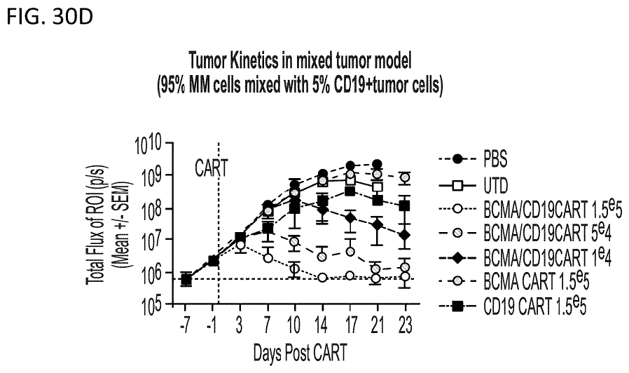

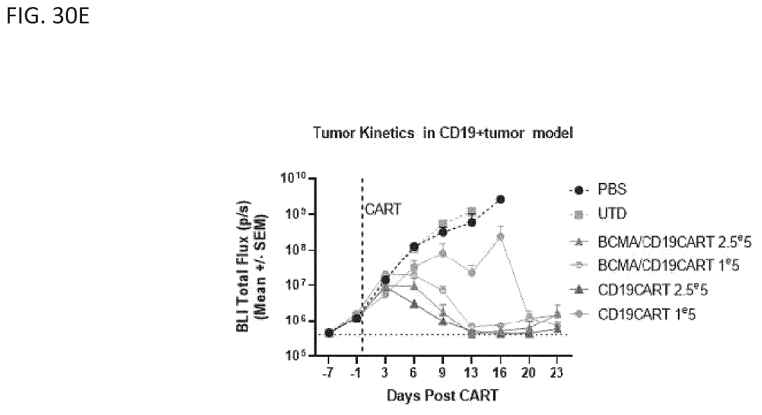

The tumor regression curves for all the groups in the three models are shown in FIGS. 30A-E. In the KMS-11-luc model, both dual targeting cocktail CART and BCMACART induced tumor regression in a dose-dependent manner as shown in FIG. 30A. At 1 e⁴ dose, both dual targeting cocktail CART and BCMACART showed delayed tumor inhibition at similar pace, and both were able to clear tumor at the end of study. Dual targeting cocktail CART exhibited more effective tumor clearance than BCMACART at 5 e⁴ dose, in which dual targeting cocktail CART cleared tumor by day 14 post CAR-T infusion, while BCMACART showed at least one-week delayed effect. In a repeated study by using the remaining cell products from the same batch, covering a wider dose ranges (1 e⁴, 5 e⁴, 1.5 e⁵) (FIG. 30B), dose dependency was confirmed in tumor regression by dual targeting cocktail CART. The regression curves of dual targeting cocktail CART at 1.5 e⁵, 5 e⁴ and BCMACART at 1.5 e⁵ overlay. BCMACART at 5 e⁴ was able to eliminate tumor by day 14 post CAR-T infusion in this cohort of mice, despite a slightly slower tumor regression than dual targeting cocktail CART at the same dose over the course of 14 days post CAR-T infusion. At 1 e⁴ dose, dual targeting cocktail CART showed better efficacy than BCMACART. Briefly, using KMS-11-luc model, it was demonstrated that dual targeting cocktail CART was able to specifically target BCMA, shown to be as potent as BCMACART in killing BCMA+ multiple myeloma tumor, and was even more potent than BCMACART at lower doses.

Next, the efficacy of dual targeting cocktail CART was evaluated when both BCMA and CD19 were present in a “mixed” model, where KMS-11-luc cells mixed with 5% of Naml-6-luc cells were implanted to mice. In the mixed tumor model as shown in FIG. 30C, only dual targeting cocktail CART at 5 e⁴ and 1 e⁴ demonstrated partial tumor inhibition in a dose dependent manner, while neither BCMACART nor CD19CART showed any effect at 5 e⁴ dose. In a repeated study covering a higher dose group as shown in FIG. 30D, it was demonstrated that dual targeting cocktail CART at 1.5 e⁵ was able to eliminate mixed tumors by day 14 post CAR-T infusion, and tumor suppression was dose dependent. In contrast, BCMACART and CD19CART at 1.5 e⁵ only showed partial tumor inhibition in this mixed model when both BCMA and CD19 were present.

Last, Nalm-6-luc model was used to show if dual targeting cocktail CART could specifically target CD19+ tumor. As indicated in FIG. 30E, dual targeting cocktail CART was also able to eliminate Nalm-6-luc tumors in a dose dependent manner comparable to monoCD19CART at a higher dose and better than monoCD19CART at a lower dose.

### Example 10: Evaluation of MOI for BCMA/CD19 CART Cellular Product Manufacturing

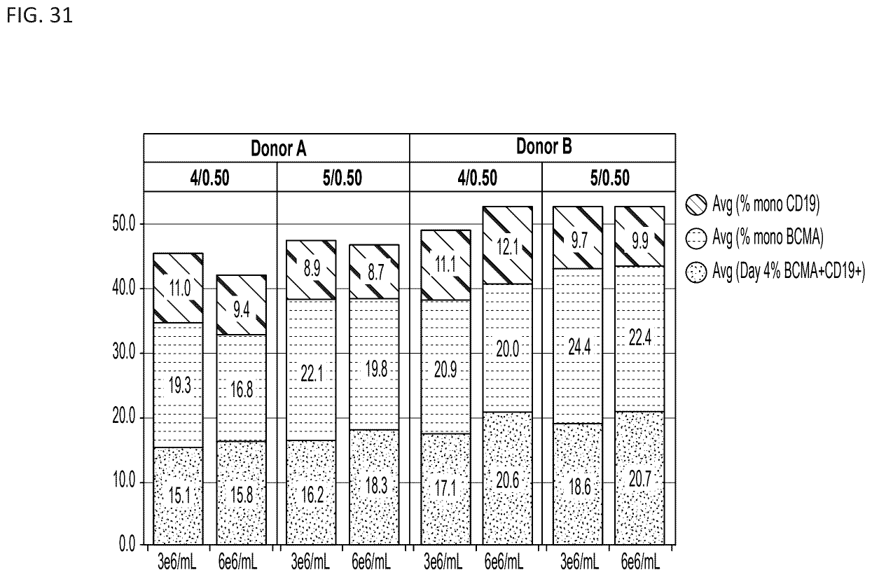

Based on the qPCR titer, a titration of the BCMA-CAR virus was performed to determine the optimum vector ratio of BCMA-CAR/CD19-CAR. Briefly, the T cells were thawed and resuspended at a density of 3×10⁶ VNCs/mL or 6×10⁶ VNCs/mL. For each MOI tested, 1 mL of the cell suspension was plated in a 24-well plate, transduced at time 0, and incubated for 20-24 hours. Each 1 mL culture was then manually washed with 3×2 mL of PBS+1% HSA. After cultivation and harvest wash, viability and cell count were assessed to determine the impact of the vector titration on final product. Harvested cells were then placed into culture at 1×10⁶VNCs/mL and CAR (BCMA and CD19) expression were measured 72 hours and 144 hours post-harvest (Day 3 and Day 6 post-harvest (PH) (Day 4 and Day 7 post transduction) Table 34 and FIG. 31).

TABLE 34
Transduction of BCMA/CD19 dual CART cellular product GMP Vector Titration

| | | MOI | | | | |
|---|---|---|---|---|---|---|
| | Day 0 | (TU/cell) | % CAR (Total BCMA-CAR = | | | |
| | seeding | BCMA- | mono BCMA population + double | | | |
| | density | CAR/CD19- | BCMA/CD19 CAR population) | | % mono CD19 CAR | |
| Donor ID | (VNC/mL) | CAR | Day 3 PH | Day 6 PH | Day 3 PH | Day 6 PH |
| Donor A | 3e6 | 4/0.5 | 34.4 | 40.6 | 11.0 | 14.4 |
| | | 5/0.5 | 38.3 | 49.1 | 8.9 | 12.1 |
| | 6e6 | 4/0.5 | 32.6 | 40.7 | 9.4 | 13.3 |
| | | 5/0.5 | 38.1 | 47.8 | 8.7 | 11.7 |
| Donor B | 3e6 | 4/0.5 | 38.0 | 47.9 | 11.1 | 14.9 |
| | | 5/0.5 | 40.6 | 52.9 | 9.7 | 12.6 |
| | 6e6 | 4/0.5 | 40.6 | 51.2 | 12.1 | 16.7 |
| | | 5/0.5 | 43.0 | 55.9 | 9.9 | 13.6 |

In some embodiments, the total number of BCMA CAR+ viable T cells, measured by flow cytometry, 4 days post transduction (or 3 days post-harvest) is used for dose related calculations. In some embodiments, T cells are seeded at a density of about 4 e6 VNC/mL in CentriCult. In some embodiments, the BCMA CAR vector is used at a MOI of 4.75 and the CD19 CAR vector is used at a MOI of 0.5.

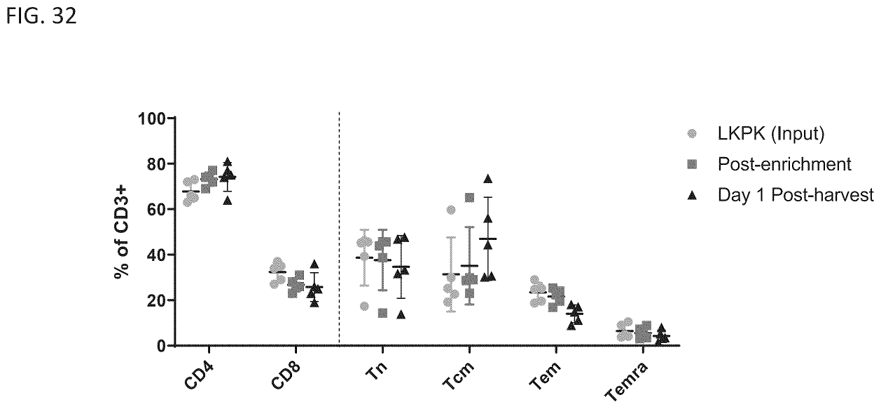

In a separate study, BCMA/CD19 CART cells were manufactured using the co-transduction rapid manufacturing approach described above at a large scale on an automated-closed system device, the CliniMACS Prodigy. Briefly, the process begins with the selection of T cells from a cryopreserved leukopak. T cells are positively selected using CD4 and CD8 microbeads. Post-selection, the T-cells are eluted into the reapplication bag and a sample is removed to assess cell concentration, viability and purity. T cells are than activated and transduced with a lentiviral vector encoding an anti-BCMA CAR and a lentiviral vector encoding an anti-CD19 CAR. As shown in FIG. 32, the T cell subsets from the output of the Prodigy did not differ from the input for the apheresis.

### Example 11: Further Characterization of T Cells Engineered to Express an Anti-BCMA CAR and an Anti-CD19 CAR

This example describes further characterization of the BCMA/CD19 CART cellular product that was generated as described in Example 9. This cellular product contains three different CAR+ populations: mono anti-BCMA CAR-T cells, mono anti-CD19 CAR-T cells, and double-positive anti-BCMA/anti-CD19 CAR-T cells. In addition, this cellular product also contains a population of untransduced T cells.

### Plasma IFN-γ in BCMA/CD19 Dual CART Cellular Product Treated Mice

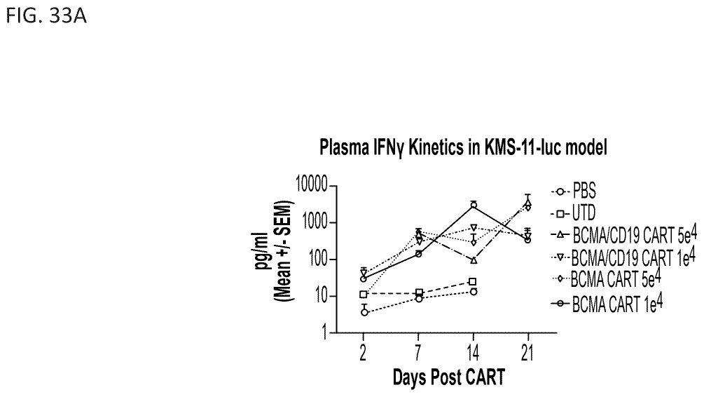

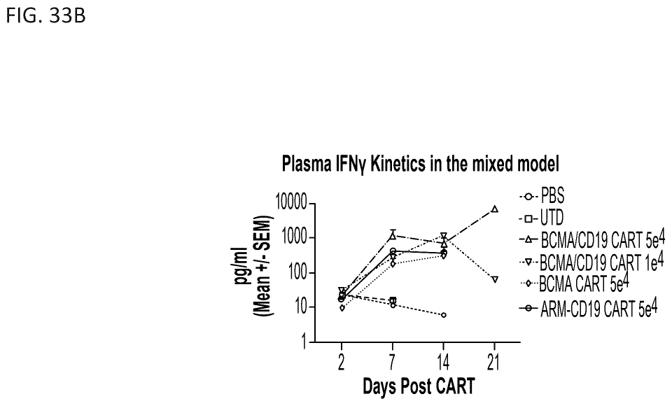

IFN-γ is a hallmark of CAR-T cell activation in response to target engagement. The kinetics of plasma IFN-γ was analyzed in the in vivo studies described in Example 9. As shown in FIGS. 33A and 33B, all CAR-T treated groups showed low levels of circulating IFN-γ (10-50 pg/ml) at day 2 and continued increasing IFN-γ secretion afterward.

### Non-Clinical Pharmacokinetics and Metabolism

The expansion of the BCMA/CD19 dual CART cellular product in peripheral blood (including mono anti-BCMA, mono anti-CD19, or double-positive anti-BCMA/anti-CD19 CAR-T cells) was analyzed by flow cytometry up to 3 weeks after infusion, and compared to the benchmarked BCMA CART in the KMS-11-luc model. Both CD3+ T cell and CAR+ T cell expansion were observed in all CAR-T treatment groups. Dose-dependent cellular expansion was observed for the BCMA/CD19 dual CART cellular product in dual or mono CAR+ T cell populations. Based on data from two studies, the BCMA/CD19 dual CART cellular product showed slightly higher expansion of BCMA targeting CAR+ T cells and total CAR+ T cells as compared to BCMA CART.

Antigen dependent expansion of the BCMA/CD19 dual CART cellular product was demonstrated by assessing expansion of CAR+ T cells in three models (KMS-11, Nalm-6, and mixed). In the BCMA expressing KMS-11 xenograft model, double-positive anti-BCMA/anti-CD19 and mono anti-BCMA CAR+ T cells expanded extensively, while in CD19 expressing Nalm-6 xenograft model, double-positive anti-BCMA/anti-CD19 and mono anti-CD19 CAR+ T cells expanded extensively. The double-positive CAR-T cell population was able to expand with activation from either BCMA or CD19 antigen alone. The initial expansion rates of double-positive anti-BCMA/anti-CD19 and mono anti-BCMA CAR+ T cells were comparable in the KMS-11 model, as were those of the double-positive CAR and mono anti-CD19 CAR+ T cells in the Nalm-6 model.

### Conclusions

The BCMA/CD19 dual CART cellular product is a novel anti-BCMA and anti-CD19 dual-targeting CAR-T cell product generated using a rapid manufacturing process, which preserves T-cell stemness.

In the three xenograft mice models (KMS-11, Nalm-6, and mixed), the BCMA/CD19 dual CART cellular product demonstrated potent in vivo pharmacology by controlling tumor growth, inducing CAR-T expansion and cytokine production, in an antigen-dependent and dose-dependent manner.

Tumor elimination in the mixed model was achieved by using the dual-targeting BCMA/CD19 CART cellular product, with increased tumor regression in a dose dependent fashion. Neither mono BCMA CART nor mono CD19 CART showed tumor regression in the mixed model. In addition, the BCMA/CD19 dual CART cellular product showed extended CAR-T expansion in vivo while the double-positive CAR population expanded with activation from either BCMA or CD19 alone. In the KMS-11-luc-model for multiple myeloma, the BCMA/CD19 dual CART cellular product showed improvement in tumor growth control at the higher dose levels tested and better tumor growth control at the lower dose level tested compared with BCMA CART.

These results support the BCMA/CD19 dual CART cellular product as a dual-targeting CAR-T that may change clinical outcomes by addressing the potential contribution of BCMA−/CD19+ stem/progenitor cells to multiple myeloma relapse, potentially providing deeper and more durable responses than traditionally manufactured or single antigen BCMA-targeting CAR-T.

### Example 12: Phase I Clinical Trial of Anti-BCMA CART Cells Manufactured Using the ARM Process

This example describes an open-label, phase I study to assess the safety and tolerability of an anti-BCMA CART cell therapy that is manufactured using the ARM process in adult patients with relapsed and/or refractory multiple myeloma.

Primary outcome measures include: incidence and nature of Dose Limiting Toxicities (DLTs) during the first 28 days after anti-BCMA CAR-T cell administration, as well as incidence and severity of adverse events (AEs) and serious adverse events (SAEs), including changes in laboratory values, ECGs, and vital signs after anti-BCMA CAR-T cell administration.

Secondary outcome measures include: manufacture success rate (defined as number of subjects treated with planned target dose divided by total number of subjects treated), ORR (proportion of subjects with the best overall response (BOR) of sCR+CR+ VGPR+PR at Months 3 and 6, as determined by local investigator using the IMWG Criteria (Kumar et al., Lancet Oncol. 2016 August; 17(8):e328-e346, herein incorporated by reference in its entirety)), CRR (proportion of subjects with the BOR of sCR+CR at Month 3, as determined by local investigator using the IMWG Criteria), DOR as assessed by local investigator (the time from achievement of sCR+CR+ VGPR+PR to relapse or death due to MM), qPCR-detected transgene of CART concentrations over time in peripheral blood and bone marrow, as well as summary of pre-existing and treatment induced immunogenicity (cellular and humoral) of BCMA CAR-T cell therapy.

### Inclusion Criteria are as Follows:

subjects with MM who are relapsed and/or refractory to at least 2 prior treatment regimens, including an IMiD (e.g. lenalidomide or pomalidomide), a proteasome inhibitor (e.g. bortezomib, carfilzomib), and an approved anti-CD38 antibody (e.g. daratumumab), if available, and have documented evidence of disease progression (IMWG criteria);
measurable disease as defined by the protocol;
ECOG performance status that is either 0 or 1 at screening;
adequate hematological values; and
must have a leukapheresis material of nonmobilized cells accepted for manufacturing.

### Exclusion Criteria are as Follows:

prior administration of a genetically modified cellular product including prior BCMA CAR-T therapy. Patients who have received prior BCMA-directed bispecific antibodies or antibody-drug conjugates (ADC) are not excluded;
autologous HSCT within 6 weeks prior to enrollment or any prior history of allogeneic hematopoietic stem cell transplant (HSCT);
chemotherapy or any concomitant anti-cancer therapies (other than protocol prescribed lymphodepletion (LD) chemotherapy) within 2 weeks prior to apheresis;
treatment with small molecule targeted antineoplastics within 2 weeks of apheresis collection or 5 half-lives whichever is shorter; and
have received antibodies or immunotherapies (other than daratumumab) within 4 weeks prior to apheresis collection. Daratumumab within 3 weeks prior to apheresis collection.

### Example 13: Evaluation of BCMA/CD19 Diabody CART

The efficacy of the CD19 antigen responsive element in the novel single chain diabody CARTs is evaluated using the NALM-6 luciferized model. 1×10⁶ NALM-6 Luc cells are implanted through lateral tail vein injection on day −7 from CART dosing. Body weight is taken, and in vivo bioluminescent imaging (BLI) is performed to evaluate tumor progression twice a week. Animals are measured twice a week, and once tumor burden reaches 3×10⁶ photon flux (photons/second), animals are randomized to their particular group (day-1). On day 0, the diabody CAR-Ts are removed from liquid nitrogen and defrosted for injection. 1×10⁶ double CAR positive cells are injected through the lateral tail vein. Experimental evaluation is conducted over the course of several weeks to determine which construct has the best functional efficacy by evaluating the decrease in BLI over time.

### EQUIVALENTS

The disclosures of each and every patent, patent application, and publication cited herein are hereby incorporated herein by reference in their entirety. While this invention has been disclosed with reference to certain embodiments, it is apparent that further embodiments and variations of this invention may be devised by others skilled in the art without departing from the true spirit and scope of the invention. The appended claims are intended to be construed to include all such embodiments and equivalent variations.

What is claimed is:

1. An isolated cell comprising:
(a) a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of:
(i) SEQ ID NOs: 86, 130, 88, 95, 131, and 132, respectively;
(ii) SEQ ID NOs: 44, 45, 84, 54, 55, and 56, respectively; or
(iii) SEQ ID NOs: 179, 180, 181, 147, 182, and 183, respectively; and
(b) a second antigen-binding domain.

2. The isolated cell of claim 1, wherein:
the first antigen-binding domain and the second antigen-binding domain are disposed in two chimeric antigen receptor (CARs), or
the first antigen-binding domain and the second antigen-binding domain are disposed in one CAR.

3. The isolated cell of claim 1 or 2, wherein the HC CDR1, HC CDR2, HC CDR3, LC CDR1, LC CDR2, and LC CDR3 comprise the amino acid sequences of SEQ ID NOs: 86, 130, 88, 95, 131, and 132, respectively.

4. The isolated cell of claim 3, wherein the HC CDR1, HC CDR2, HC CDR3, LC CDR1, LC CDR2, and LC CDR3 comprise the amino acid sequences of:
(i) SEQ ID NOs: 86, 87, 88, 95, 96, and 97, respectively;
(ii) SEQ ID NOs: 86, 109, 88, 95, 114, and 115, respectively; or
(iii) SEQ ID NOs: 86, 109, 88, 95, 114, and 97, respectively.

5. The isolated cell of claim 3 or 4, wherein:
(i) the VH comprises the amino acid sequence of SEQ ID NO: 93 or 112, or an amino acid sequence having at least about 85%, 90%, 95%, or 99% sequence identity thereto; and/or
(ii) the VH is encoded by the nucleic acid sequence of SEQ ID NO: 260, 94 or 113, or a nucleic acid sequence having at least about 85%, 90%, 95%, or 99% sequence identity thereto.

6. The isolated cell of any one of claims 3-5, wherein:
(i) the VL comprises the amino acid sequence of SEQ ID NO: 102, 118, or 124, or an amino acid sequence having at least about 85%, 90%, 95%, or 99% sequence identity thereto; and/or
(ii) the VL is encoded by the nucleic acid sequence of SEQ ID NO: 261, 103, 119, or 125, or a nucleic acid sequence having at least about 85%, 90%, 95%, or 99% sequence identity thereto.

7. The isolated cell of any one of claims 3-6, wherein the VH and VL comprise the amino acid sequences of:
(i) SEQ ID NOs: 93 and 102, respectively;
(ii) SEQ ID NOs: 112 and 118, respectively; or
(iii) SEQ ID NOs: 112 and 124, respectively.

8. The isolated cell of any one of claims 3-7, wherein:
(i) the first antigen-binding domain comprises a single-chain fragment variable (scFv) comprising the amino acid sequence of SEQ ID NO: 105, 120, or 126, or an amino acid sequence having at least about 85%, 90%, 95%, or 99% sequence identity thereto; and/or
(ii) the first antigen-binding domain is encoded by the nucleic acid sequence of SEQ ID NO: 253, 106, 121, or 127, or a nucleic acid sequence having at least about 85%, 90%, 95%, or 99% sequence identity thereto.

9. The isolated cell of any one of claims 3-8, wherein the first antigen-binding domain is disposed in a first CAR, wherein:
(i) the first CAR comprises the amino acid sequence of SEQ ID NO: 107, 226, 122, or 128, or an amino acid sequence having at least about 85%, 90%, 95%, or 99% sequence identity thereto; and/or
(ii) the first CAR is encoded by the nucleic acid sequence of SEQ ID NO: 259, 258, 108, 123, or 129, or a nucleic acid sequence having at least about 85%, 90%, 95%, or 99% sequence identity thereto.

10. The isolated cell of claim 1 or 2, wherein the HC CDR1, HC CDR2, HC CDR3, LC CDR1, LC CDR2, and LC CDR3 comprise the amino acid sequences of SEQ ID NOs: 44, 45, 84, 54, 55, and 56, respectively.

11. The isolated cell of claim 10, wherein the HC CDR1, HC CDR2, HC CDR3, LC CDR1, LC CDR2, and LC CDR3 comprise the amino acid sequences of:
(i) SEQ ID NOs: 44, 45, 76, 54, 55, and 56, respectively;
(ii) SEQ ID NOs: 44, 45, 46, 54, 55, and 56, respectively; or
(iii) SEQ ID NOs: 44, 45, 68, 54, 55, and 56, respectively.

12. The isolated cell of claim 10 or 11, wherein:
(i) the VH comprises the amino acid sequence of SEQ ID NO: 78, 52, or 70, or an amino acid sequence having at least about 85%, 90%, 95%, or 99% sequence identity thereto; and/or
(ii) the VH is encoded by the nucleic acid sequence of SEQ ID NO: 79, 53, or 71, or a nucleic acid sequence having at least about 85%, 90%, 95%, or 99% sequence identity thereto.

13. The isolated cell of any one of claims 10-12, wherein:
(i) the VL comprises the amino acid sequence of SEQ ID NO: 61, or an amino acid sequence having at least about 85%, 90%, 95%, or 99% sequence identity thereto; and/or
(ii) the VL is encoded by the nucleic acid sequence of SEQ ID NO: 62, or a nucleic acid sequence having at least about 85%, 90%, 95%, or 99% sequence identity thereto.

14. The isolated cell of any one of claims 10-13, wherein the VH and VL comprise the amino acid sequences of:
(i) SEQ ID NOs: 78 and 61, respectively;
(ii) SEQ ID NOs: 52 and 61, respectively; or
(iii) SEQ ID NOs: 70 and 61, respectively.

15. The isolated cell of any one of claims 10-14, wherein:
(i) the first antigen-binding domain comprises a single-chain fragment variable (scFv) comprising the amino acid sequence of SEQ ID NO: 80, 64, or 72, or an amino acid sequence having at least about 85%, 90%, 95%, or 99% sequence identity thereto; and/or
(ii) the first antigen-binding domain is encoded by the nucleic acid sequence of SEQ ID NO: 81, 65, or 73, or a nucleic acid sequence having at least about 85%, 90%, 95%, or 99% sequence identity thereto.

16. The isolated cell of any one of claims 10-15, wherein the first antigen-binding domain is disposed in a first CAR, wherein:
(i) the first CAR comprises the amino acid sequence of SEQ ID NO: 224, 82, 66, or 74, or an amino acid sequence having at least about 85%, 90%, 95%, or 99% sequence identity thereto; and/or
(ii) the first CAR is encoded by the nucleic acid sequence of SEQ ID NO: 83, 67, or 75, or a nucleic acid sequence having at least about 85%, 90%, 95%, or 99% sequence identity thereto.

17. The isolated cell of claim 1 or 2, wherein the HC CDR1, HC CDR2, HC CDR3, LC CDR1, LC CDR2, and LC CDR3 comprise the amino acid sequences of SEQ ID NOs: 179, 180, 181, 147, 182, and 183, respectively.

18. The isolated cell of claim 17, wherein the HC CDR1, HC CDR2, HC CDR3, LC CDR1, LC CDR2, and LC CDR3 comprise the amino acid sequences of:
(i) SEQ ID NOs: 137, 138, 139, 147, 148, and 149, respectively; or
(ii) SEQ ID NOs: 160, 161, 162, 147, 170, and 171, respectively.

19. The isolated cell of claim 17 or 18, wherein:
(i) the VH comprises the amino acid sequence of SEQ ID NO: 145 or 168, or an amino acid sequence having at least about 85%, 90%, 95%, or 99% sequence identity thereto; and/or
(ii) the VH is encoded by the nucleic acid sequence of SEQ ID NO: 146 or 169, or a nucleic acid sequence having at least about 85%, 90%, 95%, or 99% sequence identity thereto.

20. The isolated cell of any one of claims 17-19, wherein:
(i) the VL comprises the amino acid sequence of SEQ ID NO: 154 or 173, or an amino acid sequence having at least about 85%, 90%, 95%, or 99% sequence identity thereto; and/or
(ii) the VL is encoded by the nucleic acid sequence of SEQ ID NO: 155 or 174, or a nucleic acid sequence having at least about 85%, 90%, 95%, or 99% sequence identity thereto.

21. The isolated cell of any one of claims 17-20, wherein the VH and VL comprise the amino acid sequences of:
(i) SEQ ID NOs: 145 and 154, respectively, or
(ii) SEQ ID NOs: 168 and 173, respectively.

22. The isolated cell of any one of claims 17-21, wherein:
(i) the first antigen-binding domain comprises a single-chain fragment variable (scFv) comprising the amino acid sequence of SEQ ID NO: 156 or 175, or an amino acid sequence having at least about 85%, 90%, 95%, or 99% sequence identity thereto;
(ii) the first antigen-binding domain is encoded by the nucleic acid sequence of SEQ ID NO: 157 or 176, or a nucleic acid sequence having at least about 85%, 90%, 95%, or 99% sequence identity thereto.

23. The isolated cell of any one of claims 17-22, wherein the first antigen-binding domain is disposed in a first CAR, wherein:
(i) the first CAR comprises the amino acid sequence of SEQ ID NO: 158 or 177, or an amino acid sequence having at least about 85%, 90%, 95%, or 99% sequence identity thereto; and/or
(iii) the first CAR is encoded by the nucleic acid sequence of SEQ ID NO: 159 or 178, or a nucleic acid sequence having at least about 85%, 90%, 95%, or 99% sequence identity thereto.

24. An isolated cell comprising:
(a) a first antigen-binding domain which is an anti-BCMA binding domain, wherein the anti-BCMA binding domain comprises:
(i) a VH comprising a HC CDR1, HC CDR2, and HC CDR3 of an anti-BCMA sequence listed in Table 20 or 26 and a VL comprising a LC CDR1, LC CDR2, and LC CDR3 of an anti-BCMA sequence listed in Table 20 or 26, wherein the VH and VL are connected by a linker comprising the amino acid sequence of SEQ ID NO: 243;
(ii) a VH and VL comprising the amino acid sequences of SEQ ID NOs: 239 and 242, respectively, wherein the VH and VL are connected by a linker comprising the amino acid sequence of SEQ ID NO: 243; or
(iii) an scFv comprising the amino acid sequence of SEQ ID NO: 200; and
(b) a second antigen-binding domain.

25. The isolated cell of claim 24, wherein:
the first antigen-binding domain and the second antigen-binding domain are disposed in two chimeric antigen receptor (CARs), or
the first antigen-binding domain and the second antigen-binding domain are disposed in one CAR.

26. The isolated cell of any one of claims 1-25, wherein the second antigen-binding domain binds to an antigen chosen from: CD19, CD5, CD10, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, FcRn5, FcRn2, CS-1, CXCR4, 5, 7, IL-7/3R, IL7/4/3R, or IL4R, optionally wherein the B cell antigen is chosen from CD19, CD20, CD22, FcRn5, FcRn2, CS-1, CD138, CD123, CD33, CD34, CLL-1, folate receptor beta, or FLT3, optionally wherein the second antigen-binding domain binds to CD19.

27. The isolated cell of any one of claims 1-25, wherein the second antigen-binding domain binds to an antigen chosen from: EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

28. The isolated cell of any one of claims 1-25, wherein the second antigen-binding domain binds to CD19, optionally wherein:
(i) the second antigen-binding domain comprises a HC CDR1, HC CDR2, HC CDR3, LC CDR1, LC CDR2, and/or LC CDR3 of an anti-CD19 sequence listed in Table 19 or Table 22, for example, a HC CDR1, HC CDR2, HC CDR3, LC CDR1, LC CDR2, and LC CDR3 comprising the amino acid sequences of SEQ ID NOs: 295 and 245-249, respectively;
(ii) the second antigen-binding domain comprises a VH and/or VL of an anti-CD19 sequence listed in Table 19 or Table 22, for example, a VH and VL comprising the amino acid sequences of SEQ ID NOs: 250 and 251, respectively, or an amino acid sequence having at least about 85%, 90%, 95%, or 99% sequence identity thereto;
(iii) the second antigen-binding domain comprises a scFv of an anti-CD19 sequence listed in Table 19 or Table 22, for example, a scFv comprising the amino acid sequence of SEQ ID NO: 211, or an amino acid sequence having at least about 85%, 90%, 95%, or 99% sequence identity thereto; and/or
(iv) the second antigen-binding domain is disposed in a second CAR, wherein the CAR comprises a CAR of an anti-CD19 sequence listed in Table 19 or Table 22, for example, a CAR comprising the amino acid sequence of SEQ ID NO: 225 or 229, or an amino acid sequence having at least about 85%, 90%, 95%, or 99% sequence identity thereto.

29. The isolated cell of any one of claims 1-25 or 28, wherein:
(i) the first antigen-binding domain comprises a HC CDR1, HC CDR2, HC CDR3, LC CDR1, LC CDR2, and LC CDR comprising the amino acid sequences of:
(a) SEQ ID NOs: 86, 87, 88, 95, 96, and 97, respectively;
(b) SEQ ID NOs: 44, 45, 76, 54, 55, and 56, respectively; or
(c) SEQ ID NOs: 44, 45, 46, 54, 55, and 56, respectively,
and the second antigen-binding domain comprises a HC CDR1, HC CDR2, HC CDR3, LC CDR1, LC CDR2, and LC CDR3 comprising the amino acid sequences of SEQ ID NOs: 295 and 245-249, respectively;
(ii) the first antigen-binding domain comprises a VH and VL comprising the amino acid sequences of:
(a) SEQ ID NOs: 93 and 102, respectively;
(b) SEQ ID NOs: 78 and 61, respectively; or
(c) SEQ ID NOs: 52 and 61, respectively,
and the second antigen-binding domain comprises a VH and VL comprising the amino acid sequences of SEQ ID NOs: 250 and 251, respectively;
(iii) the first antigen-binding domain comprises a scFv comprising the amino acid sequence of SEQ ID NO: 105, 80, or 64, and the second antigen-binding domain comprises a scFv comprising the amino acid sequence of SEQ ID NO: 211; or
(iv) the first antigen-binding domain is encoded by the nucleic acid sequence of SEQ ID NO: 253, 106, 81, or 65, and the second antigen-binding domain is encoded by the nucleic acid sequence of SEQ ID NO: 212.

30. The isolated cell of any one of claims 1-29, wherein the first antigen-binding domain is disposed in a first CAR and the second antigen-binding domain is disposed in a second CAR, wherein the first CAR further comprises a first transmembrane domain and a first intracellular signaling domain, and/or the second CAR further comprises a second transmembrane domain and a second intracellular signaling domain.

31. The isolated cell of claim 30, wherein the first CAR is encoded by a first nucleic acid sequence and the second CAR is encoded by a second nucleic acid sequence, wherein the first and second nucleic acid sequences are disposed on separate nucleic acid molecules.

32. The isolated cell of claim 30, wherein the first CAR is encoded by a first nucleic acid sequence and the second CAR is encoded by a second nucleic acid sequence, wherein the first and second nucleic acid sequences are disposed on a single nucleic acid molecule.

33. The isolated cell of claim 32, wherein the single nucleic acid molecule comprises the following configuration in a 5′ to 3′ orientation:
(i) a nucleic acid sequence encoding the first antigen-binding domain—a nucleic acid sequence encoding a first transmembrane domain—a nucleic acid sequence encoding a first intracellular signaling domain—a nucleic acid sequence encoding a linker—a nucleic acid sequence encoding the second antigen-binding domain—a nucleic acid sequence encoding a second transmembrane domain—a nucleic acid sequence encoding a second intracellular signaling domain; or
(ii) a nucleic acid sequence encoding the second antigen-binding domain—a nucleic acid sequence encoding a second transmembrane domain—a nucleic acid sequence encoding a second intracellular signaling domain—a nucleic acid sequence encoding a linker—a nucleic acid sequence encoding the first antigen-binding domain—a nucleic acid sequence encoding a first transmembrane domain—a nucleic acid sequence encoding a first intracellular signaling domain,
optionally wherein the linker comprises a self-cleavage site, optionally wherein the linker comprises a P2A site, a T2A site, an E2A site, or an F2A site, optionally wherein the linker comprises a P2A site, optionally wherein:
the linker is encoded by the nucleic acid sequence of SEQ ID NO: 209, or a nucleic acid sequence having at least about 85%, 90%, 95%, or 99% sequence identity thereto, or
the linker comprises the amino acid sequence of SEQ ID NO: 208, or an amino acid sequence having at least about 85%, 90%, 95%, or 99% sequence identity thereto.

34. The isolated cell of claim 32 or 33, wherein:
(i) the single nucleic acid molecule comprises the nucleic acid sequence of SEQ ID NO: 215, 217, 219, 221, or 223, or a nucleic acid sequence having at least about 85%, 90%, 95%, or 99% sequence identity thereto; or
(ii) the single nucleic acid molecule encodes the amino acid sequence of SEQ ID NO: 214, 216, 218, 220, or 222, or an amino acid sequence having at least about 85%, 90%, 95%, or 99% sequence identity thereto.

35. The isolated cell of any one of claims 1-29, wherein the first antigen-binding domain and the second antigen-binding domain are disposed in one CAR, wherein the CAR further comprises a transmembrane domain and an intracellular signaling domain.

36. The isolated cell of claim 35, wherein the first antigen-binding domain comprises a first VH (VH1) and a first VL (VL1) and the second antigen-binding domain comprises a second VH (VH2) and a second VL (VL2), wherein the VH1, VL1, VH2, and VL2 are arranged in the following configuration from the N-terminus to the C-terminus:
(i) VH2—optionally linker 1 (“L1”)—VL1-optionally linker 2 (“L2”)-VH1—optionally linker 3 (“L3”)-VL2;
(ii) VH1—optionally L1-VH2—optionally L2-VL2-optionally L3-VL1;
(iii) VL2-optionally L1-VL1-optionally L2-VH1—optionally L3-VH2;
(iv) VL2-optionally L1-VH1—optionally L2-VL1-optionally L3-VH2;
(v) VH2—optionally L1-VH1—optionally L2-VL1-optionally L3-VL2;
(vi) VL1-optionally L1-VH2—optionally L2-VL2-optionally L3-VH1;
(vii) VL1-optionally L1-VL2-optionally L2-VH2—optionally L3-VH1; or
(viii) VH1—optionally L1-VL2-optionally L2-VH2—optionally L3-VL1.

37. The isolated cell of claim 36, wherein:
(i) the VH1 and VL1 comprise the amino acid sequences of:
(a) SEQ ID NOs: 93 and 102, respectively (or an amino acid sequence having at least about 85%, 90%, 95%, or 99% sequence identity thereto);
(b) SEQ ID NOs: 333 and 334, respectively (or an amino acid sequence having at least about 85%, 90%, 95%, or 99% sequence identity thereto);
(c) SEQ ID NOs: 78 and 61, respectively (or an amino acid sequence having at least about 85%, 90%, 95%, or 99% sequence identity thereto); or
(d) SEQ ID NOs: 335 and 336, respectively (or an amino acid sequence having at least about 85%, 90%, 95%, or 99% sequence identity thereto), and/or
(ii) the VH2 and VL2 comprise the amino acid sequences of:
(a) SEQ ID NOs: 250 and 251, respectively (or an amino acid sequence having at least about 85%, 90%, 95%, or 99% sequence identity thereto); or
(b) SEQ ID NOs: 331 and 332, respectively (or an amino acid sequence having at least about 85%, 90%, 95%, or 99% sequence identity thereto).

38. The isolated cell of claim 36 or 37, wherein L1 or L3 comprises the amino acid sequence of SEQ ID NO: 5, and/or L2 comprises the amino acid sequence of SEQ ID NO: 63.

39. The isolated cell of any one of claims 35-38, wherein:
(i) the CAR comprises an amino acid sequence selected from the group consisting of SEQ ID NOs: 321-330, or an amino acid sequence having at least 80, 85, 90, 95, or 99% identity thereto; or
(ii) the CAR comprises an amino acid sequence selected from the group consisting of SEQ ID NOs: 339-348, or an amino acid sequence having at least 80, 85, 90, 95, or 99% identity thereto.

40. The isolated cell of any one of claims 35-39, wherein the CAR is encoded by a nucleic acid molecule comprising the following configuration in a 5′ to 3′ orientation:
(i) a nucleic acid sequence encoding the first antigen-binding domain—optionally a nucleic acid sequence encoding a linker—a nucleic acid sequence encoding the second antigen-binding domain—a nucleic acid sequence encoding a transmembrane domain—a nucleic acid sequence encoding an intracellular signaling domain; or
(ii) a nucleic acid sequence encoding the second antigen-binding domain—optionally a nucleic acid sequence encoding a linker—a nucleic acid sequence encoding the first antigen-binding domain—a nucleic acid sequence encoding a transmembrane domain—a nucleic acid sequence encoding an intracellular signaling domain.

41. The isolated cell of any one of claims 35-40, wherein the CAR comprises the following configuration in an N- to C-orientation:
(i) the first antigen-binding domain—optionally a linker—the second antigen-binding domain—a transmembrane domain—an intracellular signaling domain; or
(ii) the second antigen-binding domain—optionally a linker—the first antigen-binding domain—a transmembrane domain—an intracellular signaling domain.

42. The isolated cell of any one of claims 1-41, wherein the first antigen-binding domain or second antigen-binding domain comprises a VH and a VL, wherein the VH and VL are connected by a linker, optionally wherein the linker comprises the amino acid sequence of SEQ ID NO: 5, 63, 104, or 243, or an amino acid sequence having at least about 85%, 90%, 95%, or 99% sequence identity thereto.

43. The isolated cell of any one of claims 30-42, wherein:
(i) the transmembrane domain, first transmembrane domain, or second transmembrane domain comprises a transmembrane domain of a protein chosen from the alpha, beta or zeta chain of T-cell receptor, CD28, CD3 epsilon, CD45, CD4, CD5, CD8, CD9, CD16, CD22, CD33, CD37, CD64, CD80, CD86, CD134, CD137 or CD154;
(ii) the transmembrane domain, first transmembrane domain, or second transmembrane domain comprises the amino acid sequence of SEQ ID NO: 6, or an amino acid sequence having at least about 85%, 90%, 95%, or 99% sequence identity thereto; or
(iii) the transmembrane domain, first transmembrane domain, or second transmembrane domain is encoded by the nucleic acid sequence of SEQ ID NO: 17, or a nucleic acid sequence having at least about 85%, 90%, 95%, or 99% sequence identity thereto.

44. The isolated cell of any one of claims 30-43, wherein the first antigen-binding domain or second antigen-binding domain is connected to the transmembrane domain, first transmembrane domain, or second transmembrane domain by a hinge region (e.g., a first or second hinge region), optionally wherein:
(i) the hinge region comprises the amino acid sequence of SEQ ID NO: 2, 3, or 4, or an amino acid sequence having at least about 85%, 90%, 95%, or 99% sequence identity thereto;
(ii) the hinge region is encoded by the nucleic acid sequence of SEQ ID NO: 13, 14, or 15, or a nucleic acid sequence having at least about 85%, 90%, 95%, or 99% sequence identity thereto;
(iii) the hinge region and the transmembrane domain comprise the amino acid sequence of SEQ ID NO: 202, or an amino acid sequence having at least about 85%, 90%, 95%, or 99% sequence identity thereto; or
(iv) the hinge region and the transmembrane domain are encoded by the nucleic acid sequence of SEQ ID NO: 203 or 213, or a nucleic acid sequence having at least about 85%, 90%, 95%, or 99% sequence identity thereto.

45. The isolated cell of any one of claims 30-44, wherein the intracellular signaling domain, first intracellular signaling domain, or second intracellular signaling domain comprises a primary signaling domain (e.g., a first or second primary signaling domain), optionally wherein:
(i) the primary signaling domain comprises a functional signaling domain derived from CD3 zeta, TCR zeta, FcR gamma, FcR beta, CD3 gamma, CD3 delta, CD3 epsilon, CD5, CD22, CD79a, CD79b, CD278 (ICOS), FcεR1, DAP10, DAP12, or CD66d;
(ii) the primary signaling domain comprises the amino acid sequence of SEQ ID NO: 9 or 10, or an amino acid sequence having at least about 85%, 90%, 95%, or 99% sequence identity thereto; or
(iii) the primary signaling domain is encoded by the nucleic acid sequence of SEQ ID NO: 20, 21, or 205, or a nucleic acid sequence having at least about 85%, 90%, 95%, or 99% sequence identity thereto.

46. The isolated cell of any one of claims 30-45, wherein the intracellular signaling domain, first intracellular signaling domain, or second intracellular signaling domain comprises a costimulatory signaling domain (e.g., a first or second costimulatory signaling domain), optionally wherein:
(i) the costimulatory signaling domain comprises a functional signaling domain derived from a MHC class I molecule, a TNF receptor protein, an Immunoglobulin-like protein, a cytokine receptor, an integrin, a signalling lymphocytic activation molecule (SLAM protein), an activating NK cell receptor, BTLA, a Toll ligand receptor, OX40, CD2, CD7, CD27, CD28, CD30, CD40, CDS, ICAM-1, 4-1BB (CD137), B7-H3, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, CD28-OX40, CD28-4-1BB, or a ligand that specifically binds with CD83;
(ii) the costimulatory signaling domain comprises the amino acid sequence of SEQ ID NO: 7, or an amino acid sequence having at least about 85%, 90%, 95%, or 99% sequence identity thereto; or
(iii) the costimulatory signaling domain is encoded by the nucleic acid sequence of SEQ ID NO: 18 or 204, or a nucleic acid sequence having at least about 85%, 90%, 95%, or 99% sequence identity thereto.

47. The isolated cell of any one of claims 30-46, wherein the intracellular signaling domain, first intracellular signaling domain, or second intracellular signaling domain comprises a functional signaling domain derived from 4-1BB and a functional signaling domain derived from CD3 zeta, optionally wherein the intracellular signaling domain, first intracellular signaling domain, or second intracellular signaling domain comprises the amino acid sequence of SEQ ID NO: 7 (or an amino acid sequence having at least about 85%, 90%, 95%, or 99% sequence identity thereto) and the amino acid sequence of SEQ ID NO: 9 or 10 (or an amino acid sequence having at least about 85%, 90%, 95%, or 99% sequence identity thereto), optionally wherein the intracellular signaling domain, first intracellular signaling domain, or second intracellular signaling domain comprises the amino acid sequence of SEQ ID NO: 7 and the amino acid sequence of SEQ ID NO: 9 or 10.

48. The isolated cell of any one of claims 30-47, wherein the CAR, first CAR, or second CAR further comprises a leader sequence (e.g., a first or second leader sequence), wherein:
(i) the leader sequence comprises the amino acid sequence of SEQ ID NO: 1, or an amino acid sequence having at least about 85%, 90%, 95%, or 99% sequence identity thereto; or
(ii) the leader sequence is encoded by the nucleic acid sequence of SEQ ID NO: 199 or 210, or a nucleic acid sequence having at least about 85%, 90%, 95%, or 99% sequence identity thereto.

49. The isolated cell of any one of claims 30-34 or 42-48, wherein:
(i) the first leader sequence and the second leader sequence are encoded by different nucleic acid sequences (e.g., differ by at least 5%, 10%, 20%, 30%, 40%, 50%, 60%, 70%, 80%, 85%, 90%, 95%, or 100%);
(ii) the first hinge region and the second hinge region are encoded by different nucleic acid sequences (e.g., differ by at least 5%, 10%, 20%, 30%, 40%, 50%, 60%, 70%, 80%, 85%, 90%, 95%, or 100%);
(iii) the first transmembrane domain and the second transmembrane domain are encoded by different nucleic acid sequences (e.g., differ by at least 5%, 10%, 20%, 30%, 40%, 50%, 60%, 70%, 80%, 85%, 90%, 95%, or 100%); and/or
(iv) the first intracellular signaling domain and the second intracellular signaling domain are encoded by different nucleic acid sequences (e.g., differ by at least 5%, 10%, 20%, 30%, 40%, 50%, 60%, 70%, 80%, 85%, 90%, 95%, or 100%), optionally wherein the first primary signaling domain and the second primary signaling domain are encoded by different nucleic acid sequences (e.g., differ by at least 5%, 10%, 20%, 30%, 40%, 50%, 60%, 70%, 80%, 85%, 90%, 95%, or 100%), and/or the first costimulatory signaling domain and the second costimulatory signaling domain are encoded by different nucleic acid sequences (e.g., differ by at least 5%, 10%, 20%, 30%, 40%, 50%, 60%, 70%, 80%, 85%, 90%, 95%, or 100%).

50. The isolated cell of claim 49, wherein:
(i) the first leader sequence and the second leader sequence comprise the same amino acid sequence (e.g., the first leader sequence and the second leader sequence comprise the amino acid sequence of SEQ ID NO: 1, or an amino acid sequence having at least about 85%, 90%, 95%, or 99% sequence identity thereto), or comprise different amino acid sequences;
(ii) the first hinge region and the second hinge region comprise the same amino acid sequence (e.g., the first hinge region and the second hinge region comprise the amino acid sequence of SEQ ID NO: 2, or an amino acid sequence having at least about 85%, 90%, 95%, or 99% sequence identity thereto), or comprise different amino acid sequences;
(iii) the first transmembrane domain and the second transmembrane domain comprise the same amino acid sequence (e.g., the first transmembrane domain and the second transmembrane domain comprise the amino acid sequence of SEQ ID NO: 6, or an amino acid sequence having at least about 85%, 90%, 95%, or 99% sequence identity thereto), or comprise different amino acid sequences; and/or
(iv) the first intracellular signaling domain and the second intracellular signaling domain comprise the same amino acid sequence, or comprise different amino acid sequences,
optionally wherein the first primary signaling domain and the second primary signaling domain comprise the same amino acid sequence (e.g., the first primary signaling domain and the second primary signaling domain comprise the amino acid sequence of SEQ ID NO: 10, or an amino acid sequence having at least about 85%, 90%, 95%, or 99% sequence identity thereto), or comprise different amino acid sequences, and/or
the first costimulatory signaling domain and the second costimulatory signaling domain comprise the same amino acid sequence (e.g., the first costimulatory signaling domain and the second costimulatory signaling domain comprise the amino acid sequence of SEQ ID NO: 7, or an amino acid sequence having at least about 85%, 90%, 95%, or 99% sequence identity thereto), or comprise different amino acid sequences (e.g., the first and second costimulatory signaling domains comprise a 4-1BB costimulatory domain sequence and a CD28 costimulatory domain sequence, respectively; or comprise a CD28 costimulatory domain sequence and a 4-1BB costimulatory domain sequence, respectively).

51. The isolated cell of claim 49 or 50, wherein:
(i) the first leader sequence and the second leader sequence are encoded by nucleic acid sequences comprising SEQ ID NOs: 199 and 210, respectively (or a nucleic acid sequence having at least about 85%, 90%, 95%, or 99% sequence identity thereto), or SEQ ID NOs: 210 and 199, respectively (or a nucleic acid sequence having at least about 85%, 90%, 95%, or 99% sequence identity thereto);
(ii) the first hinge region and the second hinge region are encoded by nucleic acid sequences comprising SEQ ID NOs: 337 and 13, respectively (or a nucleic acid sequence having at least about 85%, 90%, 95%, or 99% sequence identity thereto); or SEQ ID NOs: 13 and 337, respectively (or a nucleic acid sequence having at least about 85%, 90%, 95%, or 99% sequence identity thereto);
(iii) the first transmembrane domain and the second transmembrane domain are encoded by nucleic acid sequences comprising SEQ ID NOs: 338 and 17, respectively (or a nucleic acid sequence having at least about 85%, 90%, 95%, or 99% sequence identity thereto); or SEQ ID NOs: 17 and 338, respectively (or a nucleic acid sequence having at least about 85%, 90%, 95%, or 99% sequence identity thereto);
(iv) the first costimulatory signaling domain and the second costimulatory signaling domain are encoded by nucleic acid sequences comprising SEQ ID NOs: 204 and 18, respectively (or a nucleic acid sequence having at least about 85%, 90%, 95%, or 99% sequence identity thereto); or SEQ ID NOs: 18 and 204, respectively (or a nucleic acid sequence having at least about 85%, 90%, 95%, or 99% sequence identity thereto); and/or
(v) the first primary signaling domain and the second primary signaling domain are encoded by nucleic acid sequences comprising SEQ ID NOs: 205 and 21, respectively (or a nucleic acid sequence having at least about 85%, 90%, 95%, or 99% sequence identity thereto); or SEQ ID NOs: 21 and 205, respectively (or a nucleic acid sequence having at least about 85%, 90%, 95%, or 99% sequence identity thereto).

52. The isolated cell of any one of claims 30-51, wherein the CAR, first CAR, or second CAR is encoded by a nucleic acid molecule comprising a woodchuck hepatitis post-transcriptional regulatory element (WPRE).

53. An isolated nucleic acid molecule comprising:
(a) a first nucleic acid sequence encoding a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of:
(i) SEQ ID NOs: 86, 130, 88, 95, 131, and 132, respectively;
(ii) SEQ ID NOs: 44, 45, 84, 54, 55, and 56, respectively; or
(iii) SEQ ID NOs: 179, 180, 181, 147, 182, and 183, respectively; and
(b) a second nucleic acid sequence encoding a second antigen-binding domain.

54. The isolated nucleic acid molecule of claim 53, wherein the nucleic acid molecule comprises a first nucleic acid molecule and a second nucleic acid molecule, which are separate nucleic acid molecules, and wherein the first nucleic acid sequence is disposed on the first nucleic acid molecule and the second nucleic acid sequence is disposed on the second nucleic acid molecule.

55. An isolated nucleic acid molecule comprising:
(a) a first nucleic acid sequence encoding a first antigen-binding domain which is an anti-BCMA binding domain, wherein the anti-BCMA binding domain comprises:
(i) a VH comprising a HC CDR1, HC CDR2, and HC CDR3 of an anti-BCMA sequence listed in Table 20 or 26 and a VL comprising a LC CDR1, LC CDR2, and LC CDR3 of an anti-BCMA sequence listed in Table 20 or 26, wherein the VH and VL are connected by a linker comprising the amino acid sequence of SEQ ID NO: 243;
(ii) a VH and VL comprising the amino acid sequences of SEQ ID NOs: 239 and 242, respectively, wherein the VH and VL are connected by a linker comprising the amino acid sequence of SEQ ID NO: 243; or
(iii) an scFv comprising the amino acid sequence of SEQ ID NO: 200; and
(b) a second nucleic acid sequence encoding a second antigen-binding domain.

56. An isolated nucleic acid molecule comprising a first nucleic acid sequence encoding a first CAR and a second nucleic acid sequence encoding a second CAR, wherein the first CAR comprises a first antigen-binding domain which is an anti-BCMA binding domain, a first transmembrane domain, and a first intracellular signaling domain, and wherein the second CAR comprises a second antigen-binding domain which is an anti-CD19 binding domain, a second transmembrane domain, and a second intracellular signaling domain, wherein:
(i) the first antigen-binding domain comprises a HC CDR1, HC CDR2, HC CDR3, LC CDR1, LC CDR2, and LC CDR comprising the amino acid sequences of SEQ ID NOs: 86, 87, 88, 95, 96, and 97, respectively, and the second antigen-binding domain comprises a HC CDR1, HC CDR2, HC CDR3, LC CDR1, LC CDR2, and LC CDR comprising the amino acid sequences of SEQ ID NOs: 295 and 245-249, respectively;
(ii) the first antigen-binding domain comprises a VH and VL comprising the amino acid sequences of SEQ ID NOs: 93 and 102, respectively, and the second antigen-binding domain comprises a VH and VL comprising the amino acid sequences of SEQ ID NOs: 250 and 251, respectively;
(iii) the first antigen-binding domain comprises an scFv comprising the amino acid sequence of SEQ ID NO: 105, and the second antigen-binding domain comprises an scFv comprising the amino acid sequence of SEQ ID NO: 211;
(iv) the first CAR comprises the amino acid sequence of SEQ ID NO: 107 or 226 and the second CAR comprises the amino acid sequence of SEQ ID NO: 225 or 229; or
(v) the isolated nucleic acid molecule comprises the nucleic acid sequence of SEQ ID NO: 271.

57. An isolated nucleic acid molecule comprising a first nucleic acid sequence encoding a first CAR and a second nucleic acid sequence encoding a second CAR, wherein the first CAR comprises a first antigen-binding domain which is an anti-BCMA binding domain, a first transmembrane domain, and a first intracellular signaling domain, and wherein the second CAR comprises a second antigen-binding domain which is an anti-CD19 binding domain, a second transmembrane domain, and a second intracellular signaling domain, wherein:
(i) the first antigen-binding domain comprises a HC CDR1, HC CDR2, HC CDR3, LC CDR1, LC CDR2, and LC CDR comprising the amino acid sequences of SEQ ID NOs: 44, 45, 76, 54, 55, and 56, respectively, the second antigen-binding domain comprises a HC CDR1, HC CDR2, HC CDR3, LC CDR1, LC CDR2, and LC CDR3 comprising the amino acid sequences of SEQ ID NOs: 295 and 245-249, respectively;
(ii) the first antigen-binding domain comprises a VH and VL comprising the amino acid sequences of SEQ ID NOs: 78 and 61, respectively, and the second antigen-binding domain comprises a VH and VL comprising the amino acid sequences of SEQ ID NOs: 250 and 251, respectively;
(iii) the first antigen-binding domain comprises an scFv comprising the amino acid sequence of SEQ ID NO: 80, and the second antigen-binding domain comprises an scFv comprising the amino acid sequence of SEQ ID NO: 211;
(iv) the first CAR comprises the amino acid sequence of SEQ ID NO: 82 or 224 and the second CAR comprises the amino acid sequence of SEQ ID NO: 225 or 229; or
(v) the isolated nucleic acid molecule comprises the nucleic acid sequence of SEQ ID NO: 215.

58. An isolated polypeptide molecule encoded by the nucleic acid molecule of any one of claims 53-57.

59. An isolated CAR, wherein the CAR comprises:
(a) a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of:
(i) SEQ ID NOs: 86, 130, 88, 95, 131, and 132, respectively;
(ii) SEQ ID NOs: 44, 45, 84, 54, 55, and 56, respectively; or
(iii) SEQ ID NOs: 179, 180, 181, 147, 182, and 183, respectively; and
(b) a second antigen-binding domain.

60. An isolated CAR, wherein the CAR comprises:
(a) a first antigen-binding domain which is an anti-BCMA binding domain, wherein the anti-BCMA binding domain comprises:
(i) a VH comprising a HC CDR1, HC CDR2, and HC CDR3 of an anti-BCMA sequence listed in Table 20 or 26 and a VL comprising a LC CDR1, LC CDR2, and LC CDR3 of an anti-BCMA sequence listed in Table 20 or 26, wherein the VH and VL are connected by a linker comprising the amino acid sequence of SEQ ID NO: 243;
(ii) a VH and VL comprising the amino acid sequences of SEQ ID NOs: 239 and 242, respectively, wherein the VH and VL are connected by a linker comprising the amino acid sequence of SEQ ID NO: 243; or
(iii) an scFv comprising the amino acid sequence of SEQ ID NO: 200; and
(b) a second antigen-binding domain.

61. A vector comprising the nucleic acid molecule of any one of claims 53-57, or a nucleic acid molecule encoding the CAR of claim 59 or 60, optionally wherein the vector is chosen from a DNA vector, a RNA vector, a plasmid, a lentivirus vector, an adenoviral vector, or a retrovirus vector.

62. The vector of claim 61, further comprising an EF-1 promoter comprising the nucleic acid sequence of SEQ ID NO: 11.

63. An isolated cell comprising the nucleic acid molecule of any one of claims 53-57, the polypeptide molecule of claim 58, the CAR of claim 59 or 60, or the vector of claim 61 or 62.

64. The isolated cell of any one of claims 1-52 or 63, wherein the cell is a T cell or an NK cell.

65. A method of making a cell comprising transducing a cell with the vector of claim 62 or 63, optionally wherein the cell is a T cell or NK cell.

66. A method of making an RNA-engineered cell comprising introducing an in vitro transcribed RNA or synthetic RNA into a cell, wherein the RNA comprises the nucleic acid molecule of any one of claims 53-57, or a nucleic acid molecule encoding the CAR of claim 59 or 60, optionally wherein the cell is a T cell or NK cell.

67. A method of making a population of cells (for example, T cells) that express a chimeric antigen receptor (CAR), the method comprising:
(i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells;
(ii) contacting the population of cells (for example, T cells) with the nucleic acid molecule of any one of claims 53-57, or a nucleic acid molecule encoding the CAR of claim 59 or 60, thereby providing a population of cells (for example, T cells) comprising the nucleic acid molecule, and (iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the nucleic acid molecule in step (ii) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

68. The method of claim 67, wherein the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand), optionally wherein the agent that stimulates a CD3/TCR complex or the agent that stimulates a costimulatory molecule does not comprise a bead, optionally wherein the agent that stimulates a CD3/TCR complex comprises an anti-CD3 antibody and the agent that stimulates a costimulatory molecule comprises an anti-CD28 antibody, optionally wherein the agent that stimulates a CD3/TCR complex comprises an anti-CD3 antibody covalently attached to a colloidal polymeric nanomatrix and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™

69. The method of claim 67 or 68, wherein steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

70. The method of any one of claims 67-69, wherein steps (i) and/or (ii) are performed in serum-free cell media comprising a serum replacement, optionally wherein the serum replacement is CTS™ Immune Cell Serum Replacement (ICSR).

71. The method of any one of claims 67-70, further comprising prior to step (i):
(iv) (optionally) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider, and
(v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

72. The method of any one of claims 67-70, further comprising prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

73. The method of any one of claims 67-70, further comprising prior to step (i):
(iv) (optionally) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider, and
(v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

74. The method of any one of claims 67-73, further comprising step (vi):
culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion), optionally wherein:
step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion).

75. A method of making a population of cells (for example, T cells) that express a chimeric antigen receptor (CAR), the method comprising:
(1) contacting a population of cells (for example, T cells, for example, T cells isolated from a frozen leukapheresis product) with a cytokine chosen from IL-2, IL-7, IL-15 (for example, hetIL-15 (IL15/sIL-15Ra)), IL-21, IL-6 (for example, IL-6/sIL-6Ra), or a combination thereof,
(2) contacting the population of cells (for example, T cells) with the nucleic acid molecule of any one of claims 53-57, or a nucleic acid molecule encoding the CAR of claim 59 or 60, thereby providing a population of cells (for example, T cells) comprising the nucleic acid molecule, and
(3) harvesting the population of cells (for example, T cells) for storage (for example, reformulating the population of cells in cryopreservation media) or administration, wherein:
(a) step (2) is performed together with step (1) or no later than 5 hours after the beginning of step (1), for example, no later than 1, 2, 3, 4, or 5 hours after the beginning of step (1), and step (3) is performed no later than 26 hours after the beginning of step (1), for example, no later than 22, 23, or 24 hours after the beginning of step (1), for example, no later than 24 hours after the beginning of step (1), or
(b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1),
optionally wherein the nucleic acid molecule in step (2) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR.

76. The method of claim 75, wherein the population of cells is not contacted in vitro with an agent that stimulates a CD3/TCR complex and/or an agent that stimulates a costimulatory molecule on the surface of the cells, or if contacted, the contacting step is less than 2 hours, for example, no more than 1 or 1.5 hours.

77. The method of claim 76, wherein the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

78. The method of any one of claims 75-77, wherein steps (1) and/or (2) are performed in cell media comprising:
no more than 5, 4, 3, 2, 1, or 0% serum, optionally wherein steps (1) and/or (2) are performed in cell media comprising about 2% serum, or
a LSD1 inhibitor or a MALT1 inhibitor.

79. The method of any one of claims 75-78, further comprising receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

80. The method of any one of claims 67-79, wherein:
the population of cells at the beginning of step (i) or step (1) has been enriched for IL6R-expressing cells (for example, cells that are positive for IL6Ra and/or IL6RO); or
the population of cells at the beginning of step (i) or step (1) comprises no less than 50, 60, or 70% of IL6R-expressing cells (for example, cells that are positive for IL6Rα and/or IL6Rβ).

81. The method of any one of claims 67-80, wherein steps (i) and (ii) or steps (1) and (2) are performed in cell media comprising IL-15 (for example, hetIL-15 (IL15/sIL-15Ra)).

82. A population of cells made by the method of any one of claims 67-81.

83. A population of cells engineered to express a CAR (“a population of CAR-expressing cells”), said population comprising:
(a) about the same percentage of naïve cells, for example, naïve T cells, for example, CD45RO-CCR7+ T cells, as compared to the percentage of naïve cells, for example, naïve T cells, for example, CD45RO− CCR7+ cells, in the same population of cells prior to being engineered to express the CAR;
(b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to express the CAR;
(c) an increased percentage of naïve cells, for example, naïve T cells, for example, CD45RO-CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to express the CAR;
(d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR;
(e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR;
(f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to express the CAR;
(g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR;
(h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; or
(i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR,
wherein the population comprises a cell comprising:
(a) a first antigen-binding domain which is an anti-BCMA binding domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of:
(i) SEQ ID NOs: 86, 130, 88, 95, 131, and 132, respectively;
(ii) SEQ ID NOs: 44, 45, 84, 54, 55, and 56, respectively; or
(iii) SEQ ID NOs: 179, 180, 181, 147, 182, and 183, respectively; and
(b) a second antigen-binding domain.

84. A pharmaceutical composition comprising the cell of any one of claims 1-52, 63, or 64, or the population of cells of claim 82 or 83, and a pharmaceutically acceptable carrier.

85. A method of providing an anti-tumor immunity in a subject comprising administering to the subject an effective amount of the cell of any one of claims 1-52, 63, or 64, the population of cells of claim 82 or 83, or the pharmaceutical composition of claim 84.

86. A method of treating a subject having a disease associated with expression of BCMA comprising administering to the subject an effective amount of the cell of any one of claims 1-52, 63, or 64, the population of cells of claim 82 or 83, or the pharmaceutical composition of claim 84.

87. The method of claim 86, wherein the disease associated with BCMA expression is:
(i) a cancer or malignancy, or a precancerous condition chosen from one or more of a myelodysplasia, a myelodysplastic syndrome or a preleukemia, or
(ii) a non-cancer related indication associated with expression of BCMA.

88. The method of claim 86 or 87, wherein the disease is a hematologic cancer or a solid cancer.

89. The method of any one of claims 86-88, wherein the disease is chosen from: acute leukemia, B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell- or large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, prostate cancer (e.g., castrate-resistant or therapy-resistant prostate cancer, or metastatic prostate cancer), pancreatic cancer, lung cancer, a plasma cell proliferative disorder (e.g., asymptomatic myeloma (smoldering multiple myeloma or indolent myeloma), monoclonal gammapathy of undetermined significance (MGUS), Waldenstrom's macroglobulinemia, plasmacytoma (e.g., plasma cell dyscrasia, solitary myeloma, solitary plasmacytoma, extramedullary plasmacytoma, and multiple plasmacytoma), systemic amyloid light chain amyloidosis, or POEMS syndrome (also known as Crow-Fukase syndrome, Takatsuki disease, and PEP syndrome)), or a combination thereof.

90. The method of any one of claims 86-89, wherein the disease is multiple myeloma.

91. The method of any one of claims 85-90, wherein the population of cells or pharmaceutical composition is administered to the subject at a dose of about 1×10⁶ to about 1×10⁸ (e.g., about 2×10⁶ to about 5×10⁷, about 5×10⁶ to about 2×10⁷, about 1×10⁶ to about 1×10⁷, about 1×10⁷ to about 1×10⁸, about 1×10⁶ to about 3×10⁶, about 2×10⁶ to about 4×10⁶, about 3×10⁶ to about 5×10⁶, about 4×10⁶ to about 6×10⁶, about 5×10⁶ to about 7×10⁶, about 6×10⁶ to about 8×10⁶, about 7×10⁶ to about 9×10⁶, about 8×10⁶ to about 1×10⁷, about 9×10⁶ to about 2×10⁷, about 1×10⁷ to about 3×10⁷, about 2×10⁷ to about 4×10⁷, about 3×10⁷ to about 5×10⁷, about 4×10⁷ to about 6×10⁷, about 5×10⁷ to about 7×10⁷, about 6×10⁷ to about 8×10⁷, about 7×10⁷ to about 9×10⁷, about 8×10⁷ to about 1×10⁸, about 1×10⁶, about 2×10⁶, about 3×10⁶, about 4×10⁶, about 5×10⁶, about 6×10⁶, about 7×10⁶, about 8×10⁶, about 9×10⁶, about 1×10⁷, about 2×10⁷, about 3×10⁷, about 4×10⁷, about 5×10⁷, about 6×10⁷, about 7×10⁷, about 8×10⁷, about 9×10⁷, or about 1×10⁸) CAR-positive viable cells (e.g., BCMA CAR+ T cells),
optionally wherein the population of cells or pharmaceutical composition is administered to the subject at a dose of about 5×10⁶ to about 2×10⁷ CAR-positive viable cells (e.g., BCMA CAR+ T cells).

92. The method of any one of claims 85-91, further comprising administering to the subject a second therapeutic agent, optionally wherein the second therapeutic agent is chosen from:
(i) a PD-1 inhibitor, optionally wherein the PD-1 inhibitor is selected from the group consisting of PDR001, Nivolumab, Pembrolizumab, Pidilizumab, MEDI0680, REGN2810, TSR-042, PF-06801591, and AMP-224;
(ii) a PD-L1 inhibitor, optionally wherein the PD-L1 inhibitor is selected from the group consisting of FAZ053, Atezolizumab, Avelumab, Durvalumab, and BMS-936559;
(iii) a LAG-3 inhibitor, optionally wherein the LAG-3 inhibitor is selected from the group consisting of LAG525, BMS-986016, TSR-033, MK-4280 and REGN3767;
(iv) a TIM-3 inhibitor, optionally wherein the TIM-3 inhibitor is selected from the group consisting of MBG453, TSR-022, and LY3321367;
(v) a CTLA-4 inhibitor, optionally wherein the CTLA-4 inhibitor is Ipilimumab or Tremelimumab;
(vi) an interleukin-15 (IL-15) polypeptide, an interleukin-15 receptor alpha (IL-15Ra) polypeptide, or a combination of both an IL-15 polypeptide and an IL-15Ra polypeptide, e.g., hetIL-15;
(vii) an interleukin-12 (IL-12) polypeptide; or
(viii) an mTOR inhibitor, optionally wherein the mTOR inhibitor is RAD001 or rapamycin.

93. An isolated cell comprising:
(a) a first CAR comprising a first antigen-binding domain that binds to a first antigen, a first transmembrane domain, and a first intracellular signaling domain (e.g., a first primary signaling domain and/or a first costimulatory signaling domain), optionally wherein the first CAR further comprises a first leader sequence and/or a first hinge region; and
(b) a second CAR comprising a second antigen-binding domain that binds to a second antigen, a second transmembrane domain, and a second intracellular signaling domain (e.g., a second primary signaling domain and/or a second costimulatory signaling domain), optionally wherein the second CAR further comprises a second leader sequence and/or a second hinge region, wherein:
(i) the first leader sequence and the second leader sequence are encoded by different nucleic acid sequences (e.g., differ by at least 5%, 10%, 20%, 30%, 40%, 50%, 60%, 70%, 80%, 85%, 90%, 95%, or 100%), optionally wherein the first and second leader sequences comprise the same amino acid sequence;
(ii) the first hinge region and the second hinge region are encoded by different nucleic acid sequences (e.g., differ by at least 5%, 10%, 20%, 30%, 40%, 50%, 60%, 70%, 80%, 85%, 90%, 95%, or 100%), optionally wherein the first and second hinge regions comprise the same amino acid sequence;
(iii) the first transmembrane domain and the second transmembrane domain are encoded by different nucleic acid sequences (e.g., differ by at least 5%, 10%, 20%, 30%, 40%, 50%, 60%, 70%, 80%, 85%, 90%, 95%, or 100%), optionally wherein the first and second transmembrane domains comprise the same amino acid sequence; and/or
(iv) the first intracellular signaling domain and the second intracellular signaling domain are encoded by different nucleic acid sequences (e.g., differ by at least 5%, 10%, 20%, 30%, 40%, 50%, 60%, 70%, 80%, 85%, 90%, 95%, or 100%),
optionally wherein the first primary signaling domain and the second primary signaling domain are encoded by different nucleic acid sequences (e.g., differ by at least 5%, 10%, 20%, 30%, 40%, 50%, 60%, 70%, 80%, 85%, 90%, 95%, or 100%), and/or
the first costimulatory signaling domain and the second costimulatory signaling domain are encoded by different nucleic acid sequences (e.g., differ by at least 5%, 10%, 20%, 30%, 40%, 50%, 60%, 70%, 80%, 85%, 90%, 95%, or 100%).

94. An isolated nucleic acid molecule comprising:
(a) a first nucleic acid sequence encoding a first CAR, wherein the first CAR comprises a first antigen-binding domain that binds to a first antigen, a first transmembrane domain, and a first intracellular signaling domain (e.g., a first primary signaling domain and/or a first costimulatory signaling domain), optionally wherein the first CAR further comprises a first leader sequence and/or a first hinge region; and
(b) a second nucleic acid sequence encoding a second CAR, wherein the second CAR comprises a second antigen-binding domain that binds to a second antigen, a second transmembrane domain, and a second intracellular signaling domain (e.g., a second primary signaling domain and/or a second costimulatory signaling domain), optionally wherein the second CAR further comprises a second leader sequence and/or a second hinge region, wherein:
(i) the first leader sequence and the second leader sequence are encoded by different nucleic acid sequences (e.g., differ by at least 5%, 10%, 20%, 30%, 40%, 50%, 60%, 70%, 80%, 85%, 90%, 95%, or 100%), optionally wherein the first and second leader sequences comprise the same amino acid sequence;
(ii) the first hinge region and the second hinge region are encoded by different nucleic acid sequences (e.g., differ by at least 5%, 10%, 20%, 30%, 40%, 50%, 60%, 70%, 80%, 85%, 90%, 95%, or 100%), optionally wherein the first and second hinge regions comprise the same amino acid sequence;
(iii) the first transmembrane domain and the second transmembrane domain are encoded by different nucleic acid sequences (e.g., differ by at least 5%, 10%, 20%, 30%, 40%, 50%, 60%, 70%, 80%, 85%, 90%, 95%, or 100%), optionally wherein the first and second transmembrane domains comprise the same amino acid sequence; and/or
(iv) the first intracellular signaling domain and the second intracellular signaling domain are encoded by different nucleic acid sequences (e.g., differ by at least 5%, 10%, 20%, 30%, 40%, 50%, 60%, 70%, 80%, 85%, 90%, 95%, or 100%),
optionally wherein the first primary signaling domain and the second primary signaling domain are encoded by different nucleic acid sequences (e.g., differ by at least 5%, 10%, 20%, 30%, 40%, 50%, 60%, 70%, 80%, 85%, 90%, 95%, or 100%), and/or
the first costimulatory signaling domain and the second costimulatory signaling domain are encoded by different nucleic acid sequences (e.g., differ by at least 5%, 10%, 20%, 30%, 40%, 50%, 60%, 70%, 80%, 85%, 90%, 95%, or 100%).

95. The isolated cell of claim 93 or isolated nucleic acid molecule of claim 94, wherein the first primary signaling domain and the second primary signaling domain comprise the same amino acid sequence or comprise different amino acid sequences, and/or the first costimulatory signaling domain and the second costimulatory signaling domain comprise the same amino acid sequence or comprise different amino acid sequences (e.g., the first and second costimulatory signaling domains comprise a 4-1BB costimulatory domain sequence and a CD28 costimulatory domain sequence, respectively; or comprise a CD28 costimulatory domain sequence and a 4-1BB costimulatory domain sequence, respectively).

96. The isolated cell of claim 93 or 95 or isolated nucleic acid molecule of claim 94 or 95, wherein:
(i) the first leader sequence and the second leader sequence comprise the amino acid sequence of SEQ ID NO: 1, or an amino acid sequence having at least about 85%, 90%, 95%, or 99% sequence identity thereto;
(ii) the first hinge region and the second hinge region comprise the amino acid sequence of SEQ ID NO:
2, or an amino acid sequence having at least about 85%, 90%, 95%, or 99% sequence identity thereto;
(iii) the first transmembrane domain and the second transmembrane domain comprise the amino acid sequence of SEQ ID NO: 6, or an amino acid sequence having at least about 85%, 90%, 95%, or 99% sequence identity thereto;
(iv) the first costimulatory signaling domain and the second costimulatory signaling domain comprise the amino acid sequence of SEQ ID NO: 7, or an amino acid sequence having at least about 85%, 90%, 95%, or 99% sequence identity thereto; and/or
(v) the first primary signaling domain and the second primary signaling domain comprise the amino acid sequence of SEQ ID NO: 10, or an amino acid sequence having at least about 85%, 90%, 95%, or 99% sequence identity thereto.

97. The isolated cell of any one of claims 93, 95, or 96, or isolated nucleic acid molecule of any one of claims 94-96, wherein:
(i) the first leader sequence and the second leader sequence are encoded by nucleic acid sequences comprising SEQ ID NOs: 199 and 210, respectively (or a nucleic acid sequence having at least about 85%, 90%, 95%, or 99% sequence identity thereto), or SEQ ID NOs: 210 and 199, respectively (or a nucleic acid sequence having at least about 85%, 90%, 95%, or 99% sequence identity thereto);
(ii) the first hinge region and the second hinge region are encoded by nucleic acid sequences comprising SEQ ID NOs: 337 and 13, respectively (or a nucleic acid sequence having at least about 85%, 90%, 95%, or 99% sequence identity thereto); or SEQ ID NOs: 13 and 337, respectively (or a nucleic acid sequence having at least about 85%, 90%, 95%, or 99% sequence identity thereto);
(iii) the first transmembrane domain and the second transmembrane domain are encoded by nucleic acid sequences comprising SEQ ID NOs: 338 and 17, respectively (or a nucleic acid sequence having at least about 85%, 90%, 95%, or 99% sequence identity thereto); or SEQ ID NOs: 17 and 338, respectively (or a nucleic acid sequence having at least about 85%, 90%, 95%, or 99% sequence identity thereto);
(iv) the first costimulatory signaling domain and the second costimulatory signaling domain are encoded by nucleic acid sequences comprising SEQ ID NOs: 204 and 18, respectively (or a nucleic acid sequence having at least about 85%, 90%, 95%, or 99% sequence identity thereto); or SEQ ID NOs: 18 and 204, respectively (or a nucleic acid sequence having at least about 85%, 90%, 95%, or 99% sequence identity thereto); and/or
(v) the first primary signaling domain and the second primary signaling domain are encoded by nucleic acid sequences comprising SEQ ID NOs: 205 and 21, respectively (or a nucleic acid sequence having at least about 85%, 90%, 95%, or 99% sequence identity thereto); or SEQ ID NOs: 21 and 205, respectively (or a nucleic acid sequence having at least about 85%, 90%, 95%, or 99% sequence identity thereto).

98. The isolated cell of any one of claims 93 or 95-97, or isolated nucleic acid molecule of any one of claims 94-97, wherein the first or second antigen is chosen from: BCMA, CD19, CDS, CD10, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, FcRn5, FcRn2, CS-1, CXCR4, 5, 7, IL-7/3R, IL7/4/3R, or IL4R, optionally wherein the B cell antigen is chosen from CD19, CD20, CD22, FcRn5, FcRn2, CS-1, CD138, CD123, CD33, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

99. The isolated cell of any one of claims 93 or 95-98, or isolated nucleic acid molecule of any one of claims 94-98, wherein the first or second antigen-binding domain comprises a CDR, VH, VL, or scFv disclosed herein, or an amino acid sequence having at least about 85%, 90%, 95%, or 99% sequence identity thereto.

100. An isolated CAR, wherein the CAR comprises a first VH (VH1), a first VL (VL1), a second VH (VH2), a second VL (VL2), a transmembrane domain, and an intracellular signaling domain, wherein the VH1 ad VL1 bind to a first antigen and the VH2 and VL2 bind to a second antigen, wherein the VH1, VL1, VH2, and VL2 are arranged in the following configuration from the N-terminus to the C-terminus:
(i) VH1—optionally linker 1 (“L1”)—VH2—optionally linker 2 (“L2”)-VL2-optionally linker 3 (“L3”)-VL1;
(ii) VH1—optionally L1-VL2-optionally L2-VH2—optionally L3-VL1;
(iii) VL1-optionally L1-VH2—optionally L2-VL2-optionally L3-VH1;
(iv) VL1-optionally L1-VL2-optionally L2-VH2—optionally L3-VH1;
(v) VH2—optionally L1-VH1—optionally L2-VL1-optionally L3-VL2;
(vi) VH2—optionally L1-VL1-optionally L2-VH1—optionally L3-VL2;
(vii) VL2-optionally L1-VH1—optionally L2-VL1-optionally L3-VH2; or
(viii) VL2-optionally L1-VL1-optionally L2-VH1—optionally L3-VH2.

101. The isolated CAR of claim 100, wherein the VH1, VL1, VH2, and VL2 are arranged in the following configuration from the N-terminus to the C-terminus:
(i) VH1—linker 1 (“L1”)—VH2—linker 2 (“L2”)-VL2-linker 3 (“L3”)-VL1;
(ii) VH1-L1-VL2-L2-VH2-L3-VL1;
(iii) VL1-L1-VH2-L2-VL2-L3-VH1;
(iv) VL1-L1-VL2-L2-VH2-L3-VH1;
(v) VH2-L1-VH1-L2-VL1-L3-VL2;
(vi) VH2-L1-VL1-L2-VH1-L3-VL2;
(vii) VL2-L1-VH1-L2-VL1-L3-VH2; or
(viii) VL2-L1-VL1-L2-VH1-L3-VH2.

102. The isolated CAR of claim 100 or 101, wherein L1 or L3 comprises the amino acid sequence of SEQ ID NO: 5, and/or L2 comprises the amino acid sequence of SEQ ID NO: 63.

103. The isolated CAR of any one of claims 100-102, wherein the CAR comprises the following configuration from the N-terminus to the C-terminus:
(i) VH1—optionally linker 1 (“L1”)—VH2—optionally linker 2 (“L2”)-VL2-optionally linker 3 (“L3”)-VL1-optionally a hinge region—transmembrane domain—intracellular signaling domain;
(ii) VH1—optionally L1-VL2-optionally L2-VH2—optionally L3-VL1-optionally a hinge region—transmembrane domain—intracellular signaling domain;
(iii) VL1-optionally L1-VH2—optionally L2-VL2-optionally L3-VH1—optionally a hinge region—transmembrane domain—intracellular signaling domain;
(iv) VL1-optionally L1-VL2-optionally L2-VH2—optionally L3-VH1—optionally a hinge region—transmembrane domain—intracellular signaling domain;
(v) VH2—optionally L1-VH1—optionally L2-VL1-optionally L3-VL2-optionally a hinge region—transmembrane domain—intracellular signaling domain;
(vi) VH2—optionally L1-VL1-optionally L2-VH1—optionally L3-VL2-optionally a hinge region—transmembrane domain—intracellular signaling domain;
(vii) VL2-optionally L1-VH1—optionally L2-VL1-optionally L3-VH2—optionally a hinge region—transmembrane domain—intracellular signaling domain; or
(viii) VL2-optionally L1-VL1-optionally L2-VH1—optionally L3-VH2—optionally a hinge region—transmembrane domain—intracellular signaling domain.

104. The isolated CAR of any one of claims 100-103, wherein the first and second antigens are different and the first or second antigen is chosen from: BCMA, CD19, CD5, CD10, CD20, CD21, CD22, CD23, CD24, CD25, CD27, CD30, CD34, CD37, CD38, CD40, CD53, CD69, CD72, CD73, CD74, CD75, CD77, CD79a, CD79b, CD80, CD81, CD82, CD83, CD84, CD85, CD86, CD123, CD135, CD138, CD179, CD269, Flt3, ROR1, FcRn5, FcRn2, CS-1, CXCR4, 5, 7, IL-7/3R, IL7/4/3R, or IL4R, optionally wherein the B cell antigen is chosen from CD19, CD20, CD22, FcRn5, FcRn2, CS-1, CD138, CD123, CD33, CD34, CLL-1, folate receptor beta, FLT3, EGFRvIII, mesothelin, GD2, Tn antigen, sTn antigen, Tn-O-Glycopeptides, sTn-O-Glycopeptides, PSMA, CD97, TAG72, CD44v6, CEA, EPCAM, KIT, IL-13Ra2, leguman, GD3, CD171, IL-11Ra, PSCA, MAD-CT-1, MAD-CT-2, VEGFR2, LewisY, CD24, PDGFR-beta, SSEA-4, folate receptor alpha, ERBBs (e.g., ERBB2), Her2/neu, MUC1, EGFR, NCAM, Ephrin B2, CAIX, LMP2, sLe, HMWMAA, o-acetyl-GD2, folate receptor beta, TEM1/CD248, TEM7R, FAP, Legumain, HPV E6 or E7, ML-IAP, CLDN6, TSHR, GPRC5D, ALK, Polysialic acid, Fos-related antigen, neutrophil elastase, TRP-2, CYP1B1, sperm protein 17, beta human chorionic gonadotropin, AFP, thyroglobulin, PLAC1, globoH, RAGE1, MN-CA IX, human telomerase reverse transcriptase, intestinal carboxyl esterase, mut hsp 70-2, NA-17, NY-BR-1, UPK2, HAVCR1, ADRB3, PANX3, NY-ESO-1, GPR20, Ly6k, OR51E2, TARP, GFRα4, or a peptide of any of these antigens presented on MHC.

105. The isolated CAR of any one of claims 100-104, wherein:
(i) the VH1, VL1, VH2, or VL2 comprises a CDR, VH, or VL sequence disclosed herein, or an amino acid sequence having at least about 85%, 90%, 95%, or 99% sequence identity thereto; and/or
(ii) the hinge region, transmembrane domain, or intracellular signaling domain (e.g., a primary signaling domain and/or a costimulatory signaling domain) comprises a hinge region sequence, transmembrane domain sequence, or intracellular signaling domain sequence (e.g., a primary signaling domain sequence and/or a costimulatory signaling domain sequence) disclosed herein, or an amino acid sequence having at least about 85%, 90%, 95%, or 99% sequence identity thereto.

106. An isolated nucleic acid molecule encoding the CAR of any one of claims 100-105.

107. An isolated vector comprising the nucleic acid molecule of claim 106.

108. An isolated cell comprising the CAR of any one of claims 100-105, the nucleic acid molecule of claim 106, or the vector of claim 107.

109. A method of making a population of cells (for example, T cells) that express a chimeric antigen receptor (CAR), the method comprising:
(i) contacting (for example, binding) a population of cells (for example, T cells, for example, T cells isolated from a frozen or fresh leukapheresis product) with an agent that stimulates a CD3/TCR complex and/or an agent that stimulates a costimulatory molecule on the surface of the cells;
(ii) contacting the population of cells (for example, T cells) with a nucleic acid molecule (for example, a DNA or RNA molecule) encoding the CAR, thereby providing a population of cells (for example, T cells) comprising the nucleic acid molecule, and
(iii) harvesting the population of cells (for example, T cells) for storage (for example, reformulating the population of cells in cryopreservation media) or administration, wherein:
(a) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 (for example, 26) hours after the beginning of step (i), for example, no later than 22, 23, 24, 25, 26, 27, 28, 29, or 30 hours after the beginning of step (i), for example, no later than 24 hours after the beginning of step (i),
(b) step (ii) is performed together with step (i) or no later than 20 hours after the beginning of step (i), for example, no later than 12, 13, 14, 15, 16, 17, or 18 hours after the beginning of step (i), for example, no later than 18 hours after the beginning of step (i), and
step (iii) is performed no later than 30 hours after the beginning of step (ii), for example, no later than 22, 23, 24, 25, 26, 27, 28, 29, or 30 hours after the beginning of step (ii), or
(c) the population of cells from step (iii) are not expanded, or expanded by no more than 5, 10, 15, 20, 25, 30, 35, or 40%, for example, no more than 10%, for example, as assessed by the number of living cells, compared to the population of cells at the beginning of step (i),
optionally wherein the nucleic acid molecule in step (ii) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR,
wherein:
(I) the nucleic acid molecule comprises a first nucleic acid sequence encoding a first CAR and a second nucleic acid sequence encoding a second CAR, wherein:
the first and second nucleic acid sequences are disposed on a single nucleic acid molecule, e.g., wherein the first nucleic acid sequence and the second nucleic acid sequence are separated by a third nucleic acid sequence encoding a self-cleavage site (e.g., a P2A site, a T2A site, an E2A site, or an F2A site), or
the first and second nucleic acid sequences are disposed on separate nucleic acid molecules; or
(II) the nucleic acid molecule comprises a nucleic acid sequence encoding a CAR, wherein the CAR comprises a first VH (VH1), a first VL (VL1), a second VH (VH2), a second VL (VL2), a transmembrane domain, and an intracellular signaling domain, wherein the VH1 ad VL1 bind to a first antigen and the VH2 and VL2 bind to a second antigen, wherein the VH1, VL1, VH2, and VL2 are arranged in the following configuration from the N-terminus to the C-terminus: VH1—optionally linker 1 (“L1”)—VH2—optionally linker 2 (“L2”)-VL2-optionally linker 3 (“L3”)-VL1, VH1—optionally L1-VL2-optionally L2-VH2—optionally L3-VL1, VL1-optionally L1-VH2—optionally L2-VL2-optionally L3-VH1, VL1-optionally L1-VL2-optionally L2-VH2—optionally L3-VH1, VH2—optionally L1-VH1—optionally L2-VL1-optionally L3-VL2, VH2—optionally L1-VL1-optionally L2-VH1—optionally L3-VL2, VL2-optionally L1-VH1—optionally L2-VL1-optionally L3-VH2; or VL2-optionally L1-VL1-optionally L2-VH1—optionally L3-VH2.

110. The method of claim 109, wherein the nucleic acid molecule comprises a first nucleic acid sequence encoding a first CAR and a second nucleic acid sequence encoding a second CAR, wherein the first and second nucleic acid sequences are disposed on separate nucleic acid molecules.

111. The method of claim 110, wherein the first and second nucleic acid molecules are on separate viral vectors, and wherein step (ii) comprises transducing the population of cells (for example, T cells) with a first viral vector comprising the nucleic acid molecule encoding the first CAR and a second viral vector comprising the second nucleic acid molecule encoding the second CAR.

112. The method of claim 111, wherein the first CAR comprises an anti-BCMA binding domain (e.g., an anti-BCMA CAR) and the second CAR comprises an anti-CD19 binding domain (e.g., an anti-CD19 CAR).

113. The method of claim 112, wherein in step (ii), the population of cells is contacted with the first viral vector at a multiplicity of infection (MOI) that is higher than, equal to, or less than an MOI at which the population of cells is contacted with the second viral vector,
optionally wherein in step (ii), the population of cells is contacted with the first viral vector at a multiplicity of infection (MOI) that is higher than an MOI at which the population of cells is contacted with the second viral vector.

114. The method of claim 112 or 113, wherein in step (ii), the population of cells is contacted with the first viral vector at a first MOI and with the second viral vector at a second MOI, such that a resultant population of cells comprises a first population of cells that comprise the anti-BCMA CAR but not the anti-CD19 CAR, a second population of cells that comprise the anti-CD19 CAR but not the anti-BCMA CAR, and a third population of cells that comprise both the anti-BCMA CAR and the anti-CD19 CAR, wherein:
(i) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined;
(ii) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined; and/or
(iii) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the resultant population,
e.g., as determined by a method described in Example 10.

115. The method of claim 112 or 113, wherein in step (ii), the population of cells is contacted with:
(a) the first viral vector at an MOI of about 1 to about 10 (e.g., about 2 to about 9, about 3 to about 8, about 4 to about 7, about 5 to about 6, about 1 to about 8, about 1 to about 6, about 1 to about 4, about 8 to about 10, about 6 to about 10, about 4 to about 10, about 1 to about 3, about 2 to about 4, about 3 to about 5, about 4 to about 6, about 5 to about 7, about 6 to about 8, about 7 to about to about 9, about 8 to about 10, about 2.5 to about 5, about 1, about 2, about 3, about 4, about 5, about 6, about 7, about 8, about 9, or about 10),
optionally wherein the population of cells is contacted with the first viral vector at an MOI of about 2.5 to about 5;
(b) the second viral vector at an MOI of about 0.1 to about 5 (e.g., about 0.2 to about 4, about 0.3 to about 3, about 0.4 to about 2, about 0.5 to about 1, about 0.6 to about 0.9, about 0.7 to about 0.8, about 0.1 to about 4, about 0.1 to about 3, about 0.1 to about 2, about 0.1 to about 1, about 0.1 to about 0.5, about 4 to about 5, about 3 to about 5, about 2 to about 5, about 1 to about 5, about 0.5 to about 5, about 0.2 to about 5, about 0.1 to about 0.5, about 0.2 to about 1, about 0.5 to about 2, about 1 to about 3, about 2 to about 4, about 3 to about 5, about 0.5 to about 1, about 0.1, about 0.2, about 0.3, about 0.4, about 0.5, about 0.6, about 0.7, about 0.8, about 0.9, about 1, about 2, about 3, about 4, or about 5),
optionally wherein the population of cells is contacted with the second viral vector at an MOI of about 0.5 to about 1.0;
(c) the first viral vector at an MOI that is at least about 10% (e.g., at least about 20%, 30%, 40%, 50%, 60%, 70%, 80%, or 90%) or at least about 1 fold (e.g., at least about 2, 3, 4, 5, 6, 7, 8, 9, 10, 15, 20, 25, 30, 35, 40, 50, 60, 70, 80, 90, or 100 fold, e.g., about 2 to about 50 fold, about 3 to 20 fold, about 5 to about 15 fold, or about 8 to about 10 fold) higher than an MOI at which the population of cells is contacted with the second viral vector,
optionally wherein the first viral vector at an MOI that is about 8 to about 10 fold higher than an MOI at which the population of cells is contacted with the second viral vector; and/or
(d) the second viral vector at an MOI that is no more than 1/X, wherein X is 2, 3, 4, 5, 6, 7, 8, 9, 10, 12, 15, 20, 30, 40, 50, 60, 70. 80, 90, or 100, of an MOI at which the population of cells is contacted with the first viral vector,
optionally the second viral vector at an MOI that is no more than 1/X, wherein X is 6, 8, 10, or 12, of an MOI at which the population of cells is contacted with the first viral vector.

116. The method of any of claim 113-115, wherein in step (ii), the population of cells is contacted with:
(a) the first viral vector at an MOI of between about 4 and about 5 (e.g., about 4.75); and/or
(b) the second viral vector at an MOI between about 0.2 and about 1 (e.g., about 0.5).

117. The method of any of claims 109-116, wherein in step (ii), the population of cells comprises about 1×10⁸ to about 5×10⁹ (e.g., about 2×10⁸ to about 2×10⁹ or about 4×10⁸ to about 1×10⁹ total viable cells, optionally wherein the cells are suspended in a culture at a concentration of about 1×10⁶ to about 1×10⁷ (e.g., about 2×10⁶ to about 5×10⁶ or about 3×10⁶ to about 4×10⁶) viable cells/mL.

118. The method of any one of claims 109-117, wherein the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand), optionally wherein the agent that stimulates a CD3/TCR complex or the agent that stimulates a costimulatory molecule does not comprise a bead, optionally wherein the agent that stimulates a CD3/TCR complex comprises an anti-CD3 antibody and the agent that stimulates a costimulatory molecule comprises an anti-CD28 antibody, optionally wherein the agent that stimulates a CD3/TCR complex comprises an anti-CD3 antibody covalently attached to a colloidal polymeric nanomatrix and the agent that stimulates a costimulatory molecule comprises an anti-CD28 antibody covalently attached to a colloidal polymeric nanomatrix, optionally wherein the agent that stimulates a CD3/TCR complex and the agent that stimulates a costimulatory molecule comprise T Cell TransAct™.

119. The method of any one of claims 109-118, wherein steps (i) and/or (ii) are performed in cell media (for example, serum-free media) comprising IL-2, IL-15 (for example, hetIL-15 (IL15/sIL-15Ra)), IL-7, IL-21, IL-6 (for example, IL-6/sIL-6Ra), a LSD1 inhibitor, a MALT1 inhibitor, or a combination thereof.

120. The method of any one of claims 109-119, wherein steps (i) and/or (ii) are performed in serum-free cell media comprising a serum replacement, optionally wherein the serum replacement is CTS™ Immune Cell Serum Replacement (ICSR).

121. The method of any one of claims 109-120, further comprising prior to step (i):
(iv) (optionally) receiving a fresh leukapheresis product (or an alternative source of hematopoietic tissue such as a fresh whole blood product, a fresh bone marrow product, or a fresh tumor or organ biopsy or removal (for example, a fresh product from thymectomy)) from an entity, for example, a laboratory, hospital, or healthcare provider, and
(v) isolating the population of cells (for example, T cells, for example, CD8+ and/or CD4+ T cells) contacted in step (i) from a fresh leukapheresis product (or an alternative source of hematopoietic tissue such as a fresh whole blood product, a fresh bone marrow product, or a fresh tumor or organ biopsy or removal (for example, a fresh product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

122. The method of any one of claims 109-120, further comprising prior to step (i): receiving cryopreserved T cells isolated from a leukapheresis product (or an alternative source of hematopoietic tissue such as cryopreserved T cells isolated from whole blood, bone marrow, or tumor or organ biopsy or removal (for example, thymectomy)) from an entity, for example, a laboratory, hospital, or healthcare provider.

123. The method of any one of claims 109-120, further comprising prior to step (i):
(iv) (optionally) receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider, and
(v) isolating the population of cells (for example, T cells, for example, CD8+ and/or CD4+ T cells) contacted in step (i) from a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)), optionally wherein:
step (iii) is performed no later than 35 hours after the beginning of step (v), for example, no later than 27, 28, 29, 30, 31, 32, 33, 34, or 35 hours after the beginning of step (v), for example, no later than 30 hours after the beginning of step (v), or
the population of cells from step (iii) are not expanded, or expanded by no more than 5, 10, 15, 20, 25, 30, 35, or 40%, for example, no more than 10%, for example, as assessed by the number of living cells, compared to the population of cells at the end of step (v).

124. The method of any one of claims 109-123, further comprising step (vi):
culturing a portion of the population of cells from step (iii) for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion), optionally wherein:
step (iii) comprises harvesting and freezing the population of cells (for example, T cells) and step (vi) comprises thawing a portion of the population of cells from step (iii), culturing the portion for at least 2, 2.5, 3, 3.5, 4, 4.5, 5, 5.5, 6, 6.5, or 7 days, for example, at least 2 days and no more than 7 days, and measuring CAR expression level in the portion (for example, measuring the percentage of viable, CAR-expressing cells in the portion, for example, measuring the percentage of viable, anti-BCMA CAR-expressing cells in the portion).

125. The method of claim 124, wherein the cells from step (iii) are cultured for about two to about four days, e.g., about three days (e.g., about 72 hours following harvesting) prior to measuring CAR expression level in the portion (for example, measuring the percentage of viable, CAR-expressing cells in the portion, for example, measuring the percentage of viable, anti-BCMA CAR-expressing cells in the portion).

126. The method of claim 125, wherein the measuring of CAR expression occurs about 4 days (e.g., 96 hours) after step (ii).

127. The method of any of claims 124-126, wherein the CAR expression level is measured by flow cytometry.

128. A method of making a population of cells (for example, T cells) that express a chimeric antigen receptor (CAR), the method comprising:
(1) contacting a population of cells (for example, T cells, for example, T cells isolated from a frozen leukapheresis product) with a cytokine chosen from IL-2, IL-7, IL-15 (for example, hetIL-15 (IL15/sIL-15Ra)), IL-21, IL-6 (for example, IL-6/sIL-6Ra), or a combination thereof,
(2) contacting the population of cells (for example, T cells) with a nucleic acid molecule (for example, a DNA or RNA molecule) encoding the CAR, thereby providing a population of cells (for example, T cells) comprising the nucleic acid molecule, and
(3) harvesting the population of cells (for example, T cells) for storage (for example, reformulating the population of cells in cryopreservation media) or administration, wherein:
(a) step (2) is performed together with step (1) or no later than 5 hours after the beginning of step (1), for example, no later than 1, 2, 3, 4, or 5 hours after the beginning of step (1), and
step (3) is performed no later than 26 hours after the beginning of step (1), for example, no later than 22, 23, or 24 hours after the beginning of step (1), for example, no later than 24 hours after the beginning of step (1), or
(b) the population of cells from step (3) are not expanded, or expanded by no more than 5, 10, 15, 20, 25, 30, 35, or 40%, for example, no more than 10%, for example, as assessed by the number of living cells, compared to the population of cells at the beginning of step (1),
optionally wherein the nucleic acid molecule in step (2) is on a viral vector, optionally wherein the nucleic acid molecule in step (ii) is an RNA molecule on a viral vector, optionally wherein step (ii) comprises transducing the population of cells (for example, T cells) with a viral vector comprising a nucleic acid molecule encoding the CAR,
wherein:
(I) the nucleic acid molecule comprises a first nucleic acid sequence encoding a first CAR and a second nucleic acid sequence encoding a second CAR, wherein:
the first and second nucleic acid sequences are disposed on a single nucleic acid molecule, e.g., wherein the first nucleic acid sequence and the second nucleic acid sequence are separated by a third nucleic acid sequence encoding a self-cleavage site (e.g., a P2A site, a T2A site, an E2A site, or an F2A site), or
the first and second nucleic acid sequences are disposed on separate nucleic acid molecules; or
(II) the nucleic acid molecule comprises a nucleic acid sequence encoding a CAR, wherein the CAR comprises a first VH (VH1), a first VL (VL1), a second VH (VH2), a second VL (VL2), a transmembrane domain, and an intracellular signaling domain, wherein the VH1 ad VL1 bind to a first antigen and the VH2 and VL2 bind to a second antigen, wherein the VH1, VL1, VH2, and VL2 are arranged in the following configuration from the N-terminus to the C-terminus: VH1—optionally linker 1 (“L1”)—VH2—optionally linker 2 (“L2”)-VL2-optionally linker 3 (“L3”)-VL1, VH1—optionally L1-VL2-optionally L2-VH2—optionally L3-VL1, VL1-optionally L1-VH2—optionally L2-VL2-optionally L3-VH1, VL1-optionally L1-VL2-optionally L2-VH2—optionally L3-VH1, VH2—optionally L1-VH1—optionally L2-VL1-optionally L3-VL2, VH2—optionally L1-VL1-optionally L2-VH1—optionally L3-VL2, VL2-optionally L1-VH1—optionally L2-VL1-optionally L3-VH2; or VL2-optionally L1-VL1-optionally L2-VH1—optionally L3-VH2.

129. The method of claim 128, wherein the population of cells is not contacted in vitro with an agent that stimulates a CD3/TCR complex and/or an agent that stimulates a costimulatory molecule on the surface of the cells, or if contacted, the contacting step is less than 2 hours, for example, no more than 1 or 1.5 hours.

130. The method of claim 129, wherein the agent that stimulates a CD3/TCR complex is an agent that stimulates CD3 (for example, an anti-CD3 antibody) and wherein the agent that stimulates a costimulatory molecule is an agent that stimulates CD28, ICOS, CD27, HVEM, LIGHT, CD40, 4-1BB, OX40, DR3, GITR, CD30, TIM1, CD2, CD226, or any combination thereof, optionally wherein the agent that stimulates a CD3/TCR complex or the agent that stimulates a costimulatory molecule is chosen from an antibody (for example, a single-domain antibody (for example, a heavy chain variable domain antibody), a peptibody, a Fab fragment, or a scFv), a small molecule, or a ligand (for example, a naturally-existing, recombinant, or chimeric ligand).

131. The method of any one of claims 128-130, wherein steps (1) and/or (2) are performed in cell media comprising:
no more than 5, 4, 3, 2, 1, or 0% serum, optionally wherein steps (1) and/or (2) are performed in cell media comprising about 2% serum, or
a LSD1 inhibitor or a MALT1 inhibitor.

132. The method of any one of claims 128-131, further comprising receiving a cryopreserved leukapheresis product (or an alternative source of hematopoietic tissue such as a cryopreserved whole blood product, a cryopreserved bone marrow product, or a cryopreserved tumor or organ biopsy or removal (for example, a cryopreserved product from thymectomy)) from an entity, for example, a laboratory, hospital, or healthcare provider.

133. The method of any one of claims 109-132, wherein:
the population of cells at the beginning of step (i) or step (1) has been enriched for IL6R-expressing cells (for example, cells that are positive for IL6Rα and/or IL6Rβ); or
the population of cells at the beginning of step (i) or step (1) comprises no less than 50, 60, or 70% of IL6R-expressing cells (for example, cells that are positive for IL6Rα and/or IL6Rβ).

134. The method of any one of claims 109-133, wherein steps (i) and (ii) or steps (1) and (2) are performed in cell media comprising IL-15 (for example, hetIL-15 (IL15/sIL-15Ra)).

135. A population of cells made by the method of any one of claims 109-134.

136. A population of cells made by the method of any one of claims 112-114.

137. The population of cells of claim 136, wherein the population comprises:
a first population of cells comprising an anti-BCMA CAR but not an anti-CD19 CAR;
a second population of cells comprising an anti-CD19 CAR but not an anti-BCMA CAR; and
a third population of cells comprising both an anti-BCMA CAR and an anti-CD19 CAR.

138. The population of cells of claim 137, wherein:
(i) the total number of viable cells in the second and third populations combined is less than or equal to about 110% (e.g., less than or equal to about 105%, 100%, 90%, 80%, 70%, 60%, 50%, 40%, 30%, 20%, 10%, 5%, 1%, or less) of the total number of viable cells in the first and third populations combined;
(ii) the total number of viable cells in the first and third populations combined is greater than or equal to about 90% (e.g., greater than or equal to about 100%, 125%, 150%, 175%, 200%, 250%, 300%, 400%, 500%, 750%, 1000%, 2000%, 5000, 10000% or more) of the total number of viable cells in the second and third populations combined; and/or
(iii) the total number of viable cells in the first and third populations combined is greater than or equal to about 5% (e.g., greater than or equal to about 10%, 20%, 30%, 40%, 50%, 60%, 70%, 80%, or 90%) of the total number of viable cells in the population.

139. The population of cells of claim 137 or 138, further comprising a fourth population of cells that do not comprise a CAR.

140. A population of cells engineered to express a CAR (“a population of CAR-expressing cells”), said population comprising:
(a) about the same percentage of naïve cells, for example, naïve T cells, for example, CD45RO-CCR7+ T cells, as compared to the percentage of naïve cells, for example, naïve T cells, for example, CD45RO− CCR7+ cells, in the same population of cells prior to being engineered to express the CAR;
(b) a change within about 5% to about 10% of naïve cells, for example, naïve T cells, for example, CD45RO− CCR7+ T cells, for example, as compared to the percentage of naïve cells, for example, naïve T cells, for example, CD45RO− CCR7+ cells, in the same population of cells prior to being engineered to express the CAR;
(c) an increased percentage of naïve cells, for example, naïve T cells, for example, CD45RO-CCR7+ T cells, for example, increased by at least 1.2, 1.4, 1.6, 1.8, 2.0, 2.2, 2.4, 2.6, 2.8, or 3-fold, as compared to the percentage of naïve cells, for example, naïve T cells, for example, CD45RO− CCR7+ cells, in the same population of cells prior to being engineered to express the CAR;
(d) about the same percentage of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR;
(e) a change within about 5% to about 10% of central memory cells, for example, central memory T cells, for example, CCR7+CD45RO+ T cells, as compared to the percentage of central memory cells, for example, central memory T cells, for example, CCR7+CD45RO+ T cells, in the same population of cells prior to being engineered to express the CAR;
(f) a decreased percentage of central memory cells, for example, central memory T cells, for example, CCR7+CD45RO+ T cells, for example, decreased by at least 20, 25, 30, 35, 40, 45, or 50%, as compared to the percentage of central memory cells, for example, central memory T cells, for example, CCR7+CD45RO+ T cells, in the same population of cells prior to being engineered to express the CAR;
(g) about the same percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR;
(h) a change within about 5% to about 10% of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR; or
(i) an increased percentage of stem memory T cells, for example, CD45RA+CD95+IL-2 receptor β+CCR7+CD62L+ T cells, as compared to the percentage of stem memory T cells, for example, CD45RA+CD95+IL-2 receptor β+CCR7+CD62L+ T cells, in the same population of cells prior to being engineered to express the CAR,
wherein the population comprises a cell comprising a nucleic acid molecule (for example, a DNA or RNA molecule) encoding the CAR, wherein:
(I) the nucleic acid molecule comprises a first nucleic acid sequence encoding a first CAR and a second nucleic acid sequence encoding a second CAR, wherein:
the first and second nucleic acid sequences are disposed on a single nucleic acid molecule, e.g., wherein the first nucleic acid sequence and the second nucleic acid sequence are separated by a third nucleic acid sequence encoding a self-cleavage site (e.g., a P2A site, a T2A site, an E2A site, or an F2A site), or
the first and second nucleic acid sequences are disposed on separate nucleic acid molecules; or
(II) the nucleic acid molecule comprises a nucleic acid sequence encoding a CAR, wherein the CAR comprises a first VH (VH1), a first VL (VL1), a second VH (VH2), a second VL (VL2), a transmembrane domain, and an intracellular signaling domain, wherein the VH1 ad VL1 bind to a first antigen and the VH2 and VL2 bind to a second antigen, wherein the VH1, VL1, VH2, and VL2 are arranged in the following configuration from the N-terminus to the C-terminus: VH1—optionally linker 1 (“L1”)—VH2—optionally linker 2 (“L2”)-VL2-optionally linker 3 (“L3”)-VL1, VH1—optionally L1-VL2-optionally L2-VH2—optionally L3-VL1, VL1-optionally L1-VH2—optionally L2-VL2-optionally L3-VH1, VL1-optionally L1-VL2-optionally L2-VH2—optionally L3-VH1, VH2—optionally L1-VH1—optionally L2-VL1-optionally L3-VL2, VH2—optionally L1-VL1-optionally L2-VH1—optionally L3-VL2, VL2-optionally L1-VH1—optionally L2-VL1-optionally L3-VH2; or VL2-optionally L1-VL1-optionally L2-VH1—optionally L3-VH2.

141. An isolated cell or a population of cells made by the method of any one of claims 112-114, comprising one or more cells comprising:
(a) a first nucleic acid molecule encoding a first CAR that comprises an anti-BCMA binding domain, a first transmembrane domain, and a first intracellular signaling domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of SEQ ID NOs: 86, 87, 88, 95, 96, and 97, respectively; and
(b) a second nucleic acid molecule encoding a second CAR that comprises an anti-CD19 binding domain, a second transmembrane domain, and a second intracellular signaling domain, wherein the anti-CD19 binding domain comprises a VH comprising a HC CDR1, a HC CDR2, and a HC CDR3, and a VL comprising a LC CDR1, a LC CDR2, and a LC CDR3, wherein the HC CDR1, HC CDR2, HC CDR3, LC CDR1, LC CDR2, and LC CDR3 comprise the amino acid sequences of SEQ ID NOs:
295, 304, and 297-300, respectively.

142. An isolated cell comprising:
(a) a first nucleic acid molecule encoding a first CAR that comprises an anti-BCMA binding domain, a first transmembrane domain, and a first intracellular signaling domain, wherein the anti-BCMA binding domain comprises a heavy chain variable region (VH) comprising a heavy chain complementary determining region 1 (HC CDR1), a heavy chain complementary determining region 2 (HC CDR2), and a heavy chain complementary determining region 3 (HC CDR3), and a light chain variable region (VL) comprising a light chain complementary determining region 1 (LC CDR1), a light chain complementary determining region 2 (LC CDR2), and a light chain complementary determining region 3 (LC CDR3), wherein the HC CDR1, HC CDR2, HC CDR3, LC CDR1, LC CDR2, and LC CDR3 comprise the amino acid sequences of SEQ ID NOs: 86, 87, 88, 95, 96, and 97, respectively; and
(b) a second nucleic acid molecule encoding a second CAR that comprises an anti-CD19 binding domain, a second transmembrane domain, and a second intracellular signaling domain, wherein the anti-CD19 binding domain comprises a VH comprising a HC CDR1, a HC CDR2, and a HC CDR3, and a VL comprising a LC CDR1, a LC CDR2, and a LC CDR3, wherein the HC CDR1, HC CDR2, HC CDR3, LC CDR1, LC CDR2, and LC CDR3 comprise the amino acid sequences of SEQ ID NOs:
295, 304, and 297-300, respectively.

143. The isolated cell or population of cells of claim 141 or 142, wherein:
(i) the VH and VL of the anti-BCMA binding domain comprise the amino acid sequences of SEQ ID NOs: 93 and 102, respectively;
(ii) the VH and VL of the anti-CD19 binding domain comprise the amino acid sequences of SEQ ID NOs: 250 and 251, respectively;
(iii) the anti-BCMA binding domain comprises the amino acid sequence of SEQ ID NO: 105;
(iv) the anti-CD19 binding domain comprises the amino acid sequence of SEQ ID NO: 293;
(v) the first CAR comprises the amino acid sequence of SEQ ID NO: 107; and/or
(vi) the second CAR comprise the amino acid sequence of SEQ ID NO: 225.

144. A pharmaceutical composition comprising the cell or population of cells of any one of claims 141-143.

145. A method of providing anti-tumor immunity in a subject or treating a subject having a disease associated with expression of BCMA comprising administering to the subject an effective amount of the cell or population of cells of any one of claims 141-143 or the pharmaceutical composition of claim 144.

146. The method of claim 145, wherein the disease associated with BCMA expression is a hematologic cancer or a solid cancer.

147. The method of claim 145 or 146, wherein the disease is chosen from: acute leukemia, B-cell acute lymphoid leukemia (“BALL”), T-cell acute lymphoid leukemia (“TALL”), acute lymphoid leukemia (ALL), chronic myelogenous leukemia (CML), chronic lymphocytic leukemia (CLL), B cell prolymphocytic leukemia, blastic plasmacytoid dendritic cell neoplasm, Burkitt's lymphoma, diffuse large B cell lymphoma, follicular lymphoma, hairy cell leukemia, small cell- or large cell-follicular lymphoma, malignant lymphoproliferative conditions, MALT lymphoma, mantle cell lymphoma, Marginal zone lymphoma, multiple myeloma, myelodysplasia and myelodysplastic syndrome, non-Hodgkin's lymphoma, plasmablastic lymphoma, plasmacytoid dendritic cell neoplasm, Waldenstrom macroglobulinemia, prostate cancer (e.g., castrate-resistant or therapy-resistant prostate cancer, or metastatic prostate cancer), pancreatic cancer, lung cancer, a plasma cell proliferative disorder (e.g., asymptomatic myeloma (smoldering multiple myeloma or indolent myeloma), monoclonal gammapathy of undetermined significance (MGUS), Waldenstrom's macroglobulinemia, plasmacytoma (e.g., plasma cell dyscrasia, solitary myeloma, solitary plasmacytoma, extramedullary plasmacytoma, and multiple plasmacytoma), systemic amyloid light chain amyloidosis, or POEMS syndrome (also known as Crow-Fukase syndrome, Takatsuki disease, and PEP syndrome)), or a combination thereof.

148. The method of any one of claims 145-147, wherein the disease is multiple myeloma.
